(19) United States
(12) Patent Application Publication
(10) Pub. No.: US 2025/0179503 A1
(43) Pub. Date: Jun. 5, 2025

(54) METHODS OF USING OLIGOMERIC COMPOUNDS TO TREAT SCN2A-RELATED DISORDERS

(71) Applicant: Praxis Precision Medicines, Inc., Boston, MA (US)

(72) Inventors: Michael S. Oldham, Boston, MA (US); Steven Petrou, Eltham (AU)

(21) Appl. No.: 18/851,471

(22) PCT Filed: Mar. 28, 2023
(86) PCT No.: PCT/US2023/065074

§ 371 Date: Sep. 26, 2024

Related U.S. Application Data
(60) Provisional application No. 63/324,531, filed on Mar. 28, 2022.
(60) Provisional application No. 63/335,152, filed on Apr. 26, 2022.

(51) Int. Cl. C12N 15/113; A61P 25/08; A61P 25/28

(52) U.S. Cl. CPC C12N 15/1138; A61P 25/08; A61P 25/28; C12N 2310/11; C12N 2310/315; C12N 2310/321; C12N 2310/3231; C12N 2310/3341

(57) ABSTRACT

The human gene SCN2A encodes human SCN2A protein, the alpha-1 subunit of the voltage-gated sodium channel Nav 1.2. Mutations in SCN2A are associated with a variety of neurodevelopmental and intellectual diseases and disorders, including developmental and epileptic encephalopathy (DEE), such as early-onset DEE. Compositions and methods of using SCN2A oligomeric compounds for treating SCN2A related disorders, including early onset developmental and epileptic encephalopathy.

### RELATED APPLICATIONS

This application claims priority to U.S. Provisional Application No. 63/324,531, filed on Mar. 28, 2022, and U.S. Provisional Application No. 63/335,152, filed on Apr. 26, 2022. The entire contents of each of the foregoing applications are hereby incorporated herein by reference.

### FIELD

The present disclosure is directed, in certain aspects, to compositions and methods of using SCN2A oligomeric compounds for treating SCN2A-related disorders, including developmental and epileptic encephalopathy, such as early onset developmental and epileptic encephalopathy in a subject in need thereof.

### BACKGROUND

The human gene SCN2A encodes human SCN2A protein, the alpha-1 subunit of the voltage-gated sodium channel Nav1.2. Mutations in SCN2A are associated with a variety of neurodevelopmental and intellectual diseases and disorders, including developmental and epileptic encephalopathy (DEE), such as early-onset DEE. Early-onset DEE is caused by gain-of-function (GoF) variants in the SCN2A gene encoding the voltage-gated sodium channel Nav1.2. This is a rare, severe, and life-threatening condition characterized by drug-resistant epilepsy which is widely reported to have onset of seizures in the first three months of life (Wolff et Al 2017, Wolff et al 2019). In addition to frequent seizures, patients with early-onset DEE have a profound developmental impairment and extensive comorbidities including limitations in communication with most children being non-verbal; autonomic dysfunction; developmental delay; GI abnormalities; movement disorders, such as choreoathetosis, dystonia, ataxia; anxiety; sensory issues; urinary retention problems; and diminished quality of life resulting from (and characterized by) severe irritability, variable sleep problems (e.g., inability to fall and stay asleep), behavior issues, frequent ER visits and hospitalizations, and total reliance on caregivers. In a retrospective real-world data collection study, 10 of 15 (66.7%) early-onset DEE patients experienced an average of 4 episodes of status epilepticus in their lives, ranging from 1 to 33 episodes (Dalby et al 2021). Significant numbers of early mortality of patients with DEE, in particular early-onset patients, are noted in published cases with reported causes including Sudden Unexpected Death in Epilepsy (SUDEP), severe infections such as pneumonia, and autonomic dysfunction (Wolff et al 2019, Wolff et al 2017). Further, in an ongoing natural history study, mortality in DEE in the setting of status epilepticus or resulting from SUDEP occurred in 29% of early-onset patients (ongoing Natural History Study, data on file).

DEEs include a broad range of diseases that include neonatal and early infantile DEE, for example, Ohtahara Syndrome and epilepsy with migrating focal seizures of infancy (EIMFS); infantile and childhood DEE, for example, West Syndrome and Lennon-Gastaut Syndrome; Dravet Syndrome; Idiopathic/Generic Generalized Epilepsies (IGE/GGE); Temporal Lobe Epilepsy; Myoclonic Astatic Epilepsy (MAE); Migrating Partial Epilepsy of Infancy (MMPSI); and familial hemiplegic migraines, with or without epilepsy (Wolff, M., et al., 2019; Harkin, L. A., et al., 2007, Brain 130, 843-852; Escayg, A., et al., 2010, Epilepsia 51, 1650-1658; Miller 1.0, et al., 2007 Nov. 29 [Updated 2019 Apr. 18]. In: Adam M P, Ardinger H H, Pagon R A, et al., editors. GeneReviews® [Internet]. Seattle (WA): University of Washington, Seattle; 1993-2020).

In addition to DEE, including early-onset DEE, mutations in SCN2A are also associated with other neurodevelopmental and intellectual diseases and disorders, such as Late Seizure Onset Epileptic Encephalopathy and Benign Familial Neonatal-Infantile Seizures (BFNIS), as well as intellectual disability (ID) and/or autism spectrum disorder (ASD), with or without seizures (Wolff, M., et al., 2019, Epilepsia 60, S59-S67; Sanders, S., et al., 2018, Trends in Neurosciences 41, 442-456; Wolff, M., et al., 2017, Brain 140, 1316-1336).

Symptoms and hallmarks associated with DEEs include seizures, hypotonia, sensory issues, such as sensory integration disorders, motor development delays and dysfunctions, intellectual and cognitive dysfunctions, movement and balance dysfunctions, visual dysfunctions, delayed language and speech, gastrointestinal disorders, neurodevelopmental delays, sleep problems, and sudden unexpected death in epilepsy. Seizures include focal, clonic, tonic, and generalized tonic and clonic seizures, prolonged seizures (often lasting longer than 10 minutes), and frequent seizures (for example, convulsive, myoclonic, absence, focal, obtundation status, and tonic seizures) (Guzzetta, F., 2011, Epilepsia 52:S2, 35-38; Anwar et al., 2019, Cureus 11, e5006, Wolff et al., 2019). Symptoms and hallmarks associated with ID and ASD include motor development delays, delayed social and language milestones, repetitive actions, uncoordinated oral movements, gastrointestinal disorders, sleep problems, and seizures (Wolff et al., 2019).

There is no approved therapy specifically for the treatment of patients with early-onset DEE. The current standard of care for the treatment of seizures in early-onset DEE includes polypharmacy with multiple sodium channel blockers, along with antiseizure medications from other mechanistic classes. This approach offers limited efficacy with a significant adverse event profile. Additionally, current treatments target seizures and do not affect other aspects of the DEE phenotype, further recognizing there is a strong need for new therapies that offer improved seizure efficacy with potential for clinical benefit on other symptoms.

### SUMMARY

Provided herein are oligomeric compounds, methods, and pharmaceutical compositions for treating a SCN2A-related disorder in a subject.

In certain embodiments, the SCN2A-related disorder is early-onset developmental or epileptic encephalopathy (DEE). In certain embodiments, the SCN2A-related disorder is DEE, including, for example, Ohtahara Syndrome; epilepsy with migrating focal seizures of infancy (EIMFS); infantile and childhood DEE, for example West Syndrome and Lennon-Gastaut Syndrome; Dravet Syndrome; Idiopathic/Generic Generalized Epilepsies (IGE/GGE); Temporal Lobe Epilepsy; Myoclonic Astatic Epilepsy (MAE); Migrating Partial Epilepsy of Infancy (MMPSI); and familial hemiplegic migraines, with or without epilepsy.

In certain embodiments, the SCN2A-related disorder is late seizure onset epileptic encephalopathy. In certain embodiments, the SCN2A-related disorder is Benign Familial Neonatal-Infantile Seizures. In certain embodiments, the SCN2A-related disorder is an intellectual disability (ID). In certain embodiments, the SCN2A-related disorder is an autism spectrum disorder (ASD).

The oligomeric compounds described in this application can be used in the methods of treatment. In certain embodiments, the oligomeric compound is paired with a second oligomeric compound that is complementary to the first oligomeric compound. In certain embodiments, the oligomeric compounds is a singled-stranded oligomeric compound that is not paired to a second oligomeric compound, such as the antisense oligonucleotides described herein. In certain embodiments, the oligomeric compounds useful for treating a SCN2A-related disorder are modified oligonucleotides, such as the modified antisense oligonucleotides described herein.

Also provided are methods useful for ameliorating at least one symptom or hallmark of a SCN2A-related disorder, such as early-onset DEE, in a subject in need thereof. In certain embodiments, the symptom or hallmark includes one or more of seizures, hypotonia, sensory issues, such as sensory integration disorders, motor dysfunctions, intellectual and cognitive dysfunctions, movement and balance dysfunctions, such as choreoathetosis, dystonia, and ataxia, anxiety, sensory issues, urinary retention problems, irritability, behaviour issues, visual dysfunctions, delayed language and speech, gastrointestinal disorders (for example, gastroesophageal reflux, diarrhea, constipation, dysmotility, and the like), neurodevelopmental delays, sleep problems, sudden unexpected death in epilepsy, motor development delays, delayed social milestones, repetitive actions, uncoordinated oral movements. In certain embodiments, the seizures include focal, clonic, tonic, and generalized tonic and clonic seizures, prolonged seizures (often lasting longer than 10 minutes), and frequent seizures (for example, convulsive, myoclonic, absence, focal, obtundation status, and tonic seizures).

### BRIEF DESCRIPTION OF THE DRAWINGS

FIG. 1, panel A is a graph showing SCN2A mRNA knockdown in the brains of a R1883Q mutant mouse 14 days following intracerebroventricular administration of an exemplary oligomeric compound described herein (SCN2A ASO).

FIG. 1, panel B is a graph showing SCN2A protein knockdown in the brains of a R1883Q mutant mouse 14 days following intracerebroventricular administration of an exemplary oligomeric compound described herein (SCN2A ASO).

FIG. 2 is a graph demonstrating that a single dose of an exemplary oligomeric compound described herein (SCN2A ASO) increases survival beyond a standard of care in SCN2A GoF mice.

FIG. 3, panel A is a graph showing that a single dose of an exemplary oligomeric compound described herein (SCN2A ASO) increases survival in SCN2A GoF mice.

FIG. 3, panel B is a graph showing that re-dosing with an exemplary oligomeric compound described herein (SCN2A ASO) significantly extends survival in SCN2A GoF mice.

FIG. 4 is a graph showing that an exemplary oligomeric compound described herein (SCN2A ASO) extends survival in SCN2A GoF mice when administered after the disease onset.

FIG. 5, panel A is an illustration of IEDs detected in an 11-year-old with early onset SCN2A DEE.

FIG. 5, panel B is a bar graph showing IED burden in subjects of different ages with early onset SCN2A DEE and age-matched healthy controls.

FIG. 6 is a graph demonstrating the results of PK/PD modeling of an exemplary oligomeric compound described herein.

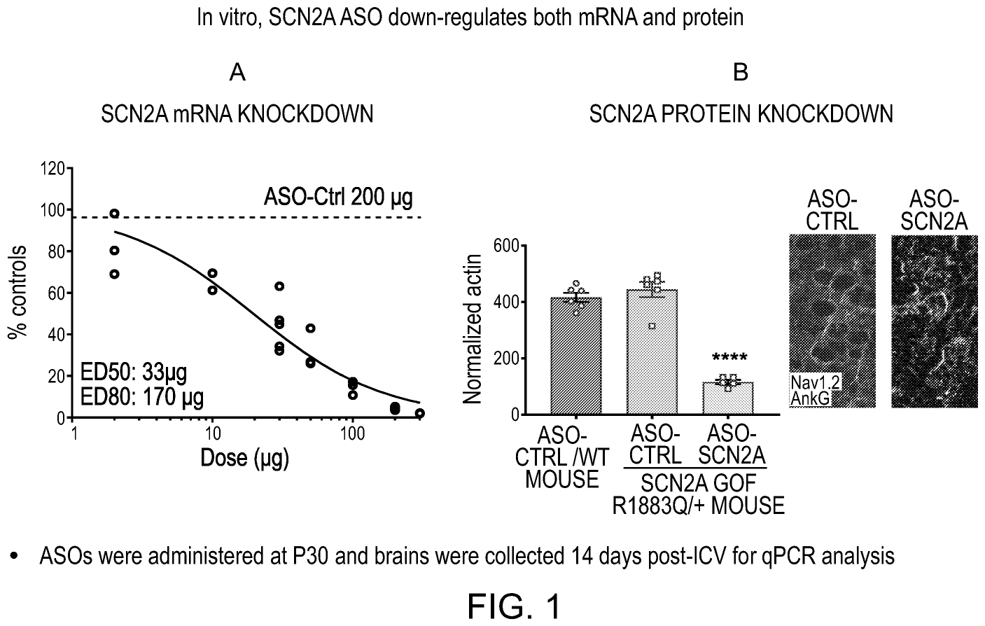

### DETAILED DESCRIPTION

It is to be understood that both the foregoing general description and the following detailed description are exemplary and explanatory only and are not restrictive. In this application, the use of the singular includes the plural unless specifically stated otherwise. As used herein, the use of “or” means “and/or” unless stated otherwise. Furthermore, the use of the term “including” as well as other forms, such as “includes” and “included,” is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, treatises, and GenBank. ENSEMBL, and NCBI reference sequence records are hereby expressly incorporated-by-reference for the portions of the document discussed herein, as well as in their entirety.

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

### Definitions

As used herein, “2′-deoxynucleoside” means a nucleoside comprising a 2′-H(H) deoxyfuranosyl sugar moiety. In certain embodiments, a 2′-deoxynucleoside is a 2′-β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside may comprise a modified nucleobase or may comprise an RNA nucleobase (uracil).

As used herein, “2′-MOE” means a 2′-OCH₂CH₂OCH₃ group in place of the 2′—OH group of a furanosyl sugar moiety. A “2′-MOE sugar moiety” means a sugar moiety with a 2′-OCH₂CH₂OCH₃ group in place of the 2′—OH group of a furanosyl sugar moiety. Unless otherwise indicated, a 2′-MOE sugar moiety is in the β-D-ribosyl configuration. “MOE” means O-methoxyethyl.

As used herein, “2′-MOE nucleoside” means a nucleoside comprising a 2′-MOE sugar moiety.

As used herein, “2′-OMe” means a 2′-OCH₃ group in place of the 2′—OH group of a furanosyl sugar moiety. A “2′-O-methyl sugar moiety” or “2′-OMe sugar moiety” means a sugar moiety with a 2′-OCH₃ group in place of the 2′—OH group of a furanosyl sugar moiety. Unless otherwise indicated, a 2′-OMe sugar moiety is in the β-D-ribosyl configuration.

As used herein, “2′-OMe nucleoside” means a nucleoside comprising a 2′-OMe sugar moiety.

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “5-methyl cytosine” means a cytosine modified with a methyl group attached to the 5 position. A 5-methyl cytosine is a modified nucleobase.

As used herein, “administering” means providing a pharmaceutical agent to a subject.

As used herein, “antisense activity” means any detectable and/or measurable change attributable to the hybridization of an antisense compound to its target nucleic acid. In certain embodiments, antisense activity is a decrease or reduction in the amount or expression of a target nucleic acid or protein encoded by such target nucleic acid compared to target nucleic acid levels or target protein levels in the absence of the antisense compound.

As used herein, “antisense compound” means an oligomeric compound capable of achieving at least one antisense activity. An antisense compound comprises an antisense oligonucleotide and optionally one or more additional features, such as a conjugate group.

As used herein, “antisense agent” means an antisense compound and optionally one or more additional features, such as a sense compound.

As used herein, “sense compound” means a sense oligonucleotide and optionally one or more additional features, such as a conjugate group.

As used herein, “antisense oligonucleotide” means an oligonucleotide, including the oligonucleotide portion of an antisense compound, that is capable of hybridizing to a target nucleic acid and is capable of at least one antisense activity. Antisense oligonucleotides include but are not limited to antisense RNAi oligonucleotides and antisense RNase H oligonucleotides.

As used herein, “ameliorate” in reference to a treatment means improvement in at least one symptom or hallmark relative to the same symptom or hallmark in the absence of the treatment. In certain embodiments, amelioration is the reduction in the severity or frequency of a symptom or hallmark or the delayed onset or slowing of progression in the severity or frequency of a symptom or hallmark. In certain embodiments, the symptom or hallmark is seizures, hypotonia, sensory issues, such as sensory integration disorders, motor dysfunctions, intellectual and cognitive dysfunctions, movement and balance dysfunctions, visual dysfunctions, delayed language and speech, gastrointestinal disorders (for example, gastroesophageal reflux, diarrhea, constipation, dysmotility, and the like), neurodevelopmental delays, sleep problems, sudden unexpected death in epilepsy, motor development delays, delayed social milestones, repetitive actions, uncoordinated oral movements. In certain embodiments, the seizures include focal, clonic, tonic, and generalized tonic and clonic seizures, prolonged seizures (often lasting longer than 10 minutes), and frequent seizures (for example, convulsive, myoclonic, absence, focal, obtundation status, and tonic seizures).

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the furanosyl sugar moiety is a ribosyl moiety. In certain embodiments, the bicyclic sugar moiety does not comprise a furanosyl moiety.

As used herein, “bicyclic nucleoside” or “BNA” means a nucleoside comprising a bicyclic sugar moiety.

As used herein, “cerebrospinal fluid” or “CSF” means the fluid filling the space around the brain and spinal cord. “Artificial cerebrospinal fluid” or “aCSF” means a prepared or manufactured fluid that has certain properties of cerebrospinal fluid.

As used herein, “cleavable moiety” means a bond or group of atoms that is cleaved under physiological conditions, for example, inside a cell, an animal, or a human.

As used herein, “complementary” in reference to an oligonucleotide means that at least 70% of the nucleobases of the oligonucleotide or one or more portions thereof and the nucleobases of a another nucleic acid or one or more portions thereof are capable of hydrogen bonding with one another when the nucleobase sequence of the oligonucleotide and the other nucleic acid are aligned in opposing directions. As used herein, complementary nucleobases means nucleobases that are capable of forming hydrogen bonds with one another. Complementary nucleobase pairs include adenine (A) and thymine (T), adenine (A) and uracil (U), cytosine (C) and guanine (G), 5-methyl cytosine (mC) and guanine (G). Complementary oligonucleotides and/or target nucleic acids need not have nucleobase complementarity at each nucleoside. Rather, some mismatches are tolerated. As used herein, “fully complementary” or “100% complementary” in reference to an oligonucleotide, or a portion thereof, means that the oligonucleotide, or portion thereof, is complementary to another oligonucleotide or target nucleic acid at each nucleobase of the shorter of the two oligonucleotides, or at each nucleoside if the oligonucleotides are the same length.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “cEt” means a 4′ to 2′ bridge in place of the 2′OH-group of a ribosyl sugar moiety, wherein the bridge has the formula of 4′-CH(CH₃)—O-2′, and wherein the methyl group of the bridge is in the S configuration. A “cEt sugar moiety” is a bicyclic sugar moiety with a 4′ to 2′ bridge in place of the 2′OH-group of a ribosyl sugar moiety, wherein the bridge has the formula of 4′-CH(CH₃)—O-2′, and wherein the methyl group of the bridge is in the S configuration. “cEt” means constrained ethyl.

As used herein, “cEt nucleoside” means a nucleoside comprising a cEt modified sugar moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “chirally controlled” in reference to an internucleoside linkage means chirality at that linkage is enriched for a particular stereochemical configuration.

As used herein, “deoxy region” means a region of 5-12 contiguous nucleotides, wherein at least 70% of the nucleosides are 2′-β-D-deoxynucleosides. In certain embodiments, each nucleoside is selected from a 2′-β-D-deoxynucleoside, a bicyclic nucleoside, and a 2′-substituted nucleoside. In certain embodiments, a deoxy region supports RNase H activity. In certain embodiments, a deoxy region is the gap or internal region of a gapmer.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings” or “wing segments.” In certain embodiments, the internal region is a deoxy region. The positions of the internal region or gap refer to the order of the nucleosides of the internal region and are counted starting from the 5′-end of the internal region. Unless otherwise indicated, “gapmer” refers to a sugar motif. In certain embodiments, each nucleoside of the gap is a 2′-β-D-deoxynucleoside. In certain embodiments, the gap comprises one 2′-substituted nucleoside at position 1, 2, 3, 4, or 5 of the gap, and the remainder of the nucleosides of the gap are 2′-β-D-deoxynucleosides. As used herein, the term “MOE gapmer” indicates a gapmer having a gap comprising 2′-β-D-deoxynucleosides and wings comprising 2′-MOE nucleosides. As used herein, the term “mixed wing gapmer” indicates a gapmer having wings comprising modified nucleosides comprising at least two different sugar modifications. Unless otherwise indicated, a gapmer may comprise one or more modified internucleoside linkages and/or modified nucleobases and such modifications do not necessarily follow the gapmer pattern of the sugar modifications.

As used herein, “hotspot region” is a range of nucleobases on a target nucleic acid that is amenable to oligomeric compound-mediated reduction of the amount or activity of the target nucleic acid.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, the term “in some embodiments,” “in other embodiments,” or the like, refers to embodiments of all aspects of the disclosure, unless the context clearly indicates otherwise.

As used herein, “internucleoside linkage” means the covalent linkage between contiguous nucleosides in an oligonucleotide. As used herein, “modified internucleoside linkage” means any internucleoside linkage other than a phosphodiester internucleoside linkage. “Phosphorothioate internucleoside linkage” or “PS internucleoside linkage” is a modified internucleoside linkage in which one of the non-bridging oxygen atoms of a phosphodiester internucleoside linkage is replaced with a sulfur atom.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “LNA” means locked nucleic acid. An “LNA sugar moiety” is a bicyclic sugar moiety with a 4′ to 2′ bridge in place of the 2′OH-group of a furanosyl sugar moiety, wherein the bridge has the formula of 4′-CH₂—O-2′. “LNA” means locked nucleic acid. In some embodiments, the furanosyl sugar moiety is a ribosyl sugar moiety. As used herein, “LNA nucleoside” means a nucleoside comprising a LNA sugar moiety.

As used herein, “non-bicyclic modified sugar moiety” means a modified sugar moiety that comprises a modification, such as a substituent, that does not form a bridge between two atoms of the sugar to form a second ring.

As used herein, “mismatch” or “non-complementary” means a nucleobase of a first oligonucleotide that is not complementary with the corresponding nucleobase of a second oligonucleotide or target nucleic acid when the first and second oligonucleotide are aligned.

As used herein, “motif” means the pattern of unmodified and/or modified sugar moieties, nucleobases, and/or internucleoside linkages, in an oligonucleotide.

As used herein, “nucleobase” means an unmodified nucleobase or a modified nucleobase. As used herein an “unmodified nucleobase” is adenine (A), thymine T), cytosine (C), uracil (U), or guanine (G). As used herein, a “modified nucleobase” is a group of atoms other than unmodified A, T, C, U, or G capable of pairing with at least one unmodified nucleobase. A “5-methyl cytosine” is a modified nucleobase. A universal base is a modified nucleobase that can pair with any one of the five unmodified nucleobases. As used herein, “nucleobase sequence” means the order of contiguous nucleobases in a target nucleic acid or oligonucleotide independent of any sugar or internucleoside linkage modification.

As used herein, “nucleoside” means a compound or a fragment of a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a contiguous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.” As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to a subject. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution or sterile artificial cerebrospinal fluid.

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein, “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein, “prodrug” means a therapeutic agent in a form outside the body that is converted to a different form within a subject or cells thereof. Typically, conversion of a prodrug within the subject is facilitated by the action of an enzymes (e.g., endogenous or viral enzyme) or chemicals present in cells or tissues and/or by physiologic conditions.

As used herein, “reducing the amount,” “reducing the activity,” “decreasing the amount,” or “decreasing the activity” refers to a reduction or blockade of the transcriptional expression or activity relative to the transcriptional expression or activity in an untreated or control sample and does not necessarily indicate a total elimination of transcriptional expression or activity.

As used herein, “RNA” means an RNA transcript and includes pre-mRNA and mature mRNA unless otherwise specified.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense compounds that act through RNase H.

As used herein, the term “SCN2A-related disorder”, means a disease or disorder associated with a mutation in the SCN2A gene, including neurodevelopmental and intellectual diseases and disorders, such as developmental and epileptic encephalopathy (DEE), including early-onset DEE, Ohtahara Syndrome and epilepsy with migrating focal seizures of infancy (EIMFS); infantile and childhood DEE, for example West Syndrome and Lennon-Gastaut Syndrome; Dravet Syndrome; Idiopathic/Generic Generalized Epilepsies (IGE/GGE); Temporal Lobe Epilepsy; Myoclonic Astatic Epilepsy (MAE); Migrating Partial Epilepsy of Infancy (MMPSI); and familial hemiplegic migraines, with or without epilepsy. Other SCN2A disorders include Late Seizure Onset Epileptic Encephalopathy and Benign Familial Neonatal-Infantile Seizures (BFNIS), as well as intellectual disability (ID) and/or autism spectrum disorder (ASD), with or without seizures.

As used herein, the term “early onset developmental and epileptic encephalopathy” encompasses a SCN2A-related disorder as defined herein.

As used herein, “self-complementary” in reference to an oligonucleotide means an oligonucleotide that at least partially hybridizes to itself.

As used herein, “standard in vitro assay” means the assay described in Example 1 and reasonable variations thereof.

As used herein, “standard in vivo assay” means the assay described in Example 8 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “subject” means a human or non-human animal. In certain embodiments, the subject is a human.

As used herein, “sugar moiety” means an unmodified sugar moiety or a modified sugar moiety. As used herein, “unmodified sugar moiety” means a 2′-OH(H) β-D-ribosyl moiety, as found in RNA (an “unmodified RNA sugar moiety”), or a 2′-H(H) β-D-deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests. In certain embodiments, a hallmark is apparent on a brain MRI scan.

As used herein, “target nucleic acid” and “target RNA” mean a nucleic acid that an antisense compound is designed to affect. Target RNA means an RNA transcript and includes pre-mRNA and mature mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “therapeutically effective amount” means an amount of a pharmaceutical agent that provides a therapeutic benefit to a subject. For example, a therapeutically effective amount improves a symptom or hallmark of a disease or disorder.

As used herein, “treat”, “treating” and “treatment” refer to improving a subject's disease or disorder by administering an oligomeric agent or oligomeric compound described herein. In some embodiments, “treat”, “treating” and “treatment” refer to reducing the severity of the disease or disorder in a subject, including retarding or slowing the progression of the disease or disorder (“therapeutic treatment”) or achieving beneficial or desired clinical results. The beneficial or desired clinical results include, but are not limited to, alleviation of symptoms; diminishment of the extent of a disease or disorder; stabilized (i.e., not worsening) state of the disease or disorder, delay in onset or slowing of the disease or disorder progression; amelioration of the disease or disorder state or remission (whether partial or total), whether detectable or undetectable; an amelioration of at least one measurable physical parameter, not necessarily discernible by the subject; and enhancement or improvement of the disease or disorder. Treatment includes eliciting a clinically-significant response without excessive levels of side effects. Treatment also includes prolonging survival as compared to expected survival if not receiving treatment. In certain embodiments, treating a subject improves a symptom relative to the same symptom in the absence of the treatment. In certain embodiments, treatment reduces in the severity or frequency of a symptom, or delays the onset of a symptom, slows the progression of a symptom, or slows the severity or frequency of a symptom.

As used herein, the terms “prevent,” “preventing” and “prevention” contemplate an action that occurs before a subject begins to suffer from the specified disease, disorder or condition (“prophylactic treatment”).

### I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

### A. Certain Modified Nucleosides

Modified nucleosides comprise a modified sugar moiety or a modified nucleobase or both a modified sugar moiety and a modified nucleobase.

### 1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non-bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE” or “O-methoxyethyl”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ), where each Rₘ and Rₙ is, independently, H, an amino protecting group, or substituted or unsubstituted C₁-C₁₀ alkyl.

In certain embodiments, a 2′-substituted non-bicyclic modified nucleoside comprises a sugar moiety comprising a non-bridging 2′-substituent group selected from: F, OCF₃. OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO 2019/157531, incorporated by reference herein. A 2′-modified sugar moiety has an additional stereocenter at the 2′-position relative to a 2′-deoxyfuranosyl sugar moiety; therefore, such sugar moieties have a total of sixteen possible isomeric configurations. 2′-modified sugar moieties described herein are in the β-D-ribosyl isomeric configuration unless otherwise specified.

Certain modified sugar moieties comprise a substituent that bridges two atoms of the furanosyl ring to form a second ring, resulting in a bicyclic sugar moiety. Nucleosides comprising such bicyclic sugar moieties have been referred to as bicyclic nucleosides (BNAs), locked nucleosides, or conformationally restricted nucleotides (CRN). Certain such compounds are described in US Patent Publication No. 2013/0190383; and PCT publication WO 2013/036868. In certain such embodiments, the bicyclic sugar moiety comprises a bridge between the 4′ and the 2′ furanose ring atoms. In certain such embodiments, the furanose ring is a ribose ring. Examples of such 4′ to 2′ bridging sugar substituents include but are not limited to: 4′-CH₂-2′, 4′—(CH₂)₂-2′, 4′—(CH₂)₃-2′, 4′—CH₂—O-2′ (“LNA”), 4′-CH₂—S-2′, 4′—(CH₂)₂-0-2′ (“ENA”), 4′-CH(CH₃)—O-2′ (referred to as “constrained ethyl” or “cEt” when in the S configuration), 4′-CH₂—O—CH₂-2′, 4′—CH₂—N(R)-2′, 4′—CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′—C(RₐR_{b})—O—N(R)-2′, 4′—CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672). In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRa)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J1), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Alback et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207, Imanishi et al., U.S. Pat. No. 6,268,490, Imanishi et al. U.S. Pat. No. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; Allerson et al., US2008/0039618; and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the G-L configuration or in the β-D configuration.

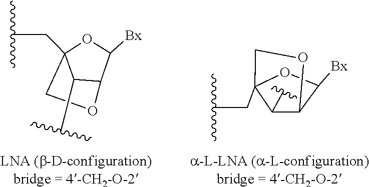

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

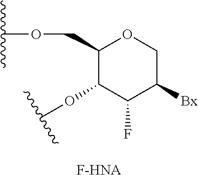

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

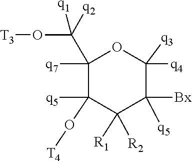

wherein, independently, for each of the modified THP nucleosides:
Bx is a nucleobase moiety;
T₃ and T₄ are each, independently, an internucleoside linking group linking the modified THP nucleoside to the remainder of an oligonucleotide or one of T1 and T4 is an internucleoside linking group linking the modified THP nucleoside to the remainder of an oligonucleotide and the other of T₃ and T₄ is H, a hydroxyl protecting group, a linked conjugate group, or a 5′ or 3′-terminal group;
q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

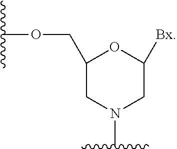

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

### 2. Certain Modified Nucleobases

In certain embodiments, modified oligonucleotides comprise one or more nucleosides comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

### 3. Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphodiesters, which contain a phosphodiester bond (“P(O₂)═O”) (also referred to as unmodified or naturally occurring linkages), phosphotriesters, methylphosphonates, phosphoramidates, phosphorothioates (“P(O₂)═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphodiester internucleoside linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate internucleoside linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate internucleoside linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkage in a particular, independently selected stereochemical configuration. In certain embodiments, the particular configuration of the particular phosphorothioate internucleoside linkage is present in at least 65% of the molecules in the population. In certain embodiments, the particular configuration of the particular phosphorothioate internucleoside linkage is present in at least 70% of the molecules in the population. In certain embodiments, the particular configuration of the particular phosphorothioate internucleoside linkage is present in at least 80% of the molecules in the population. In certain embodiments, the particular configuration of the particular phosphorothioate internucleoside linkage is present in at least 90% of the molecules in the population. In certain embodiments, the particular configuration of the particular phosphorothioate internucleoside linkage is present in at least 99% of the molecules in the population. Such chirally enriched populations of modified oligonucleotides can be generated using synthetic methods known in the art, e.g., methods described in Oka et al., JACS 2003, 125, 8307, Wan et al. Nuc. Acid. Res., 2014, 42, 13456, and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

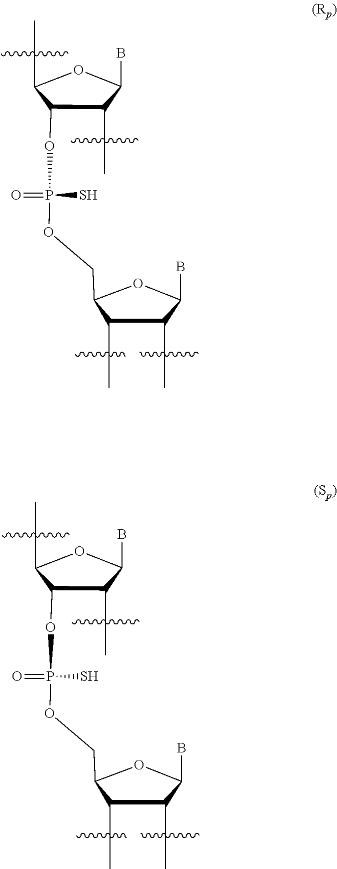

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl (MOP), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, oligonucleotides comprise one or more type of modified sugar and/or unmodified sugar moiety arranged along the oligonucleotide or portion thereof in a defined pattern or sugar motif. In certain instances, such sugar motifs include but are not limited to any of the sugar modifications discussed herein.

In certain embodiments, modified oligonucleotides have a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-6 nucleosides. In certain embodiments, each nucleoside of each wing of a gapmer comprises a modified sugar moiety. In certain embodiments, at least one nucleoside of each wing of a gapmer comprises a modified sugar moiety. In certain embodiments, at least two nucleosides of each wing of a gapmer comprise a modified sugar moiety. In certain embodiments, at least three nucleosides of each wing of a gapmer comprise a modified sugar moiety. In certain embodiments, at least four nucleosides of each wing of a gapmer comprise a modified sugar moiety. In certain embodiments, at least five nucleosides of each wing of a gapmer comprise a modified sugar moiety.

In certain embodiments, the gap of a gapmer comprises 7-12 nucleosides. In certain embodiments, each nucleoside of the gap of a gapmer comprises a 2′-deoxyribosyl sugar moiety. In certain embodiments, at least six nucleosides of the gap of a gapmer comprise a 2′-β-D-deoxyribosyl sugar moiety. In certain embodiments, each nucleoside of the gap of a gapmer comprises a 2′-β-D-deoxyribosyl sugar moiety. In certain embodiments, at least one nucleoside of the gap of a gapmer comprises a modified sugar moiety. In certain embodiments, at least one nucleoside of the gap of a gapmer comprises a 2′-OMe sugar moiety.

In certain embodiments, the gapmer is a deoxy gapmer. In certain embodiments, the nucleosides on the gap side of each wing/gap junction comprise 2′-deoxyribosyl sugar moieties and the nucleosides on the wing sides of each wing/gap junction comprise modified sugar moieties. In certain embodiments, at least six nucleosides of the gap of a gapmer comprise a 2′-β-D-deoxyribosyl sugar moiety. In certain embodiments, each nucleoside of the gap of a gapmer comprises a 2′-deoxyribosyl sugar moiety. In certain embodiments, each nucleoside of each wing of a gapmer comprises a modified sugar moiety. In certain embodiments, one nucleoside of the gap comprises a modified sugar moiety and each remaining nucleoside of the gap comprises a 2′-deoxyribosyl sugar moiety.

In certain embodiments, modified oligonucleotides comprise or consist of a portion having a fully modified sugar motif. In such embodiments, each nucleoside of the fully modified portion of the modified oligonucleotide comprises a modified sugar moiety. In certain embodiments, each nucleoside of the entire modified oligonucleotide comprises a modified sugar moiety. In certain embodiments, modified oligonucleotides comprise or consist of a portion having a fully modified sugar motif, wherein each nucleoside within the fully modified portion comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified oligonucleotide comprises the same 2′-modification.

Herein, the lengths (number of nucleosides) of the three regions of a gapmer may be provided using the notation [# of nucleosides in the 5′-wing]-[# of nucleosides in the gap]-[# of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in each sugar moiety of each wing and the gap nucleosides comprises a 2′-β-D-deoxyribosyl sugar moiety. Thus, a 5-10-5 MOE gapmer consists of 5 linked 2′-MOE nucleosides in the 5′-wing, 10 linked 2′-β-D-deoxynucleosides in the gap, and 5 linked 2′-MOE nucleosides in the 3′-wing. A 3-10-3 cEt gapmer consists of 3 linked cEt nucleosides in the 5′-wing, 10 linked 2′-β-D-deoxynucleosides in the gap, and 3 linked cEt nucleosides in the 3′-wing. A 5-8-5 gapmer consists of 5 linked nucleosides comprising a modified sugar moiety in the 5′-wing, 8 linked 2′-β-D-deoxynucleosides in the gap, and 5 linked nucleosides comprising a modified sugar moiety in the 3′-wing. A mixed wing gapmer has at least two different modified sugar moieties in the 5′- and/or the 3′-wing. A 5-8-5 or 5-8-4 mixed wing gapmer has at least two different modified sugar moieties in the 5′- and/or the 3′-wing.

In certain embodiments, modified oligonucleotides are 5-10-5 MOE gapmers. In certain embodiments, modified oligonucleotides are 4-10-6 MOE gapmers. In certain embodiments, modified oligonucleotides are 6-10-4 MOE gapmers. In certain embodiments, modified oligonucleotides are 4-8-6 MOE gapmers. In certain embodiments, modified oligonucleotides are 6-8-4 MOE gapmers. In certain embodiments, modified oligonucleotides are 5-8-5 MOE gapmers. In certain embodiments, modified oligonucleotides are X—Y—Z MOE gapmers, wherein X and Z are independently selected from 1, 2, 3, 4, 5, 6, or 7 linked 2′-MOE nucleosides and Y is selected from 7, 8, 9, 10, or 11 linked deoxynucleosides.

In certain embodiments, modified oligonucleotides have the following sugar motif (5′ to 3′): eeeeedyddddddddeeeee, eeeeeddddddddddeeeee, eeeeeeddddddddddeeee, eeeeddddddddddeeeeee, eeeeddddddddeeeeee, eeeeeeddddddddeeee, or eeeeeddddddddeeeee, wherein ‘d’ represents a 2′-deoxyribosyl sugar moiety, ‘e’ represents a 2′-MOE sugar moiety, and ‘y’ represents a 2′-OMe sugar moiety.

### 2. Certain Nucleobase Motifs

In certain embodiments, oligonucleotides comprise modified and/or unmodified nucleobases arranged along the oligonucleotide or portion thereof in a defined pattern or motif. In certain embodiments, each nucleobase is modified. In certain embodiments, none of the nucleobases is modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of the nucleoside is a 2′-β-D-deoxyribosyl sugar moiety. In certain embodiments, the modified nucleobase is selected from: a 2-thiopyrimidine and a 5-propynepyrimidine.

### 3. Certain Internucleoside Linkage Motifs

In certain embodiments, oligonucleotides comprise modified and/or unmodified internucleoside linkages arranged along the oligonucleotide or portion thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester internucleoside linkage is not a terminal internucleoside linkage, and the remaining internucleoside linkages are phosphorothioate internucleoside linkages. In certain such embodiments, all of the phosphorothioate internucleoside linkages are stereorandom. In certain embodiments, all of the phosphorothioate internucleoside linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

In certain embodiments, modified oligonucleotides have an internucleoside linkage motif of (5′ to 3′): sooosssssssssssssss, soooossssssssssooss, sooooossssssssssoss, sooossssssssssoooss, soosssssssssoooss, soooosssssssssoss, or sooosssssssssooss, wherein each “s” represents a phosphorothioate internucleoside linkage and each “o” represents a phosphodiester internucleoside linkage.

### C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target nucleic acid in an oocyte injection model. Oligonucleotides 25 nucleobases in length with 8 or 11 mismatch bases near the ends of the oligonucleotides were able to direct specific cleavage of the target nucleic acid, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 27, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

### D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

### E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a portion of an oligonucleotide has a nucleobase sequence that is complementary to a second oligonucleotide or an identified reference nucleic acid, such as a target nucleic acid. In certain embodiments, the nucleobase sequence of a portion or entire length of an oligonucleotide is at least 50%, at least 60%, at least 70%, at least 80%, at least 85%, at least 90%, at least 95%, at least 97%, at least 98%, at least 99%, or 100% complementary to the second oligonucleotide or nucleic acid, such as a target nucleic acid.

### II. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, abasic nucleosides, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge, and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a N-acetylgalactosamine (GalNAc) cluster (e.g., WO2014/179620).

In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In certain embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

### 1. Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, lipophilic groups, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain oligomeric compounds, a conjugate moiety is attached to an oligonucleotide via a more complex conjugate linker comprising one or more conjugate linker moieties, which are sub-units making up a conjugate linker. In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate or phosphodiester linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphodiester internucleoside linkage and covalently attached to the remainder of the conjugate linker or conjugate moiety by a phosphate or phosphorothioate internucleoside linkage. In certain such embodiments, the cleavable moiety is 2′-deoxyadenosine.

### 3. Cell-Targeting Moieties

In certain embodiments, a conjugate group comprises a cell-targeting moiety. In certain embodiments, a conjugate group has the general formula:

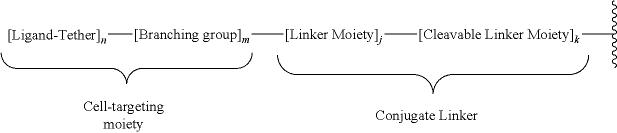

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

### B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

### III. Oligomeric Duplexes

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a portion complementary to a target nucleic acid and a second oligomeric compound having a portion complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

### IV. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or subject.

### V. Certain Target Nucleic Acids

In certain embodiments, oligomeric compounds comprise or consist of an oligonucleotide comprising a portion that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic, and untranslated regions. In certain embodiments, the target nucleic acid is a mature mRNA. In certain embodiments, the target nucleic acid is a pre-mRNA. In certain embodiments, the target region is entirely within an intron. In certain embodiments, the target region spans an intron/exon junction. In certain embodiments, the target region is at least 50% within an intron.

### A. Complementarity/Mismatches to the Target Nucleic Acid

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a portion that is 100% or fully complementary to a target nucleic acid. In certain embodiments, the portion of full complementarity is 6, 7, 8, 9, 10, 11, 12, 13, 14, 15, 16, 17, 18, 19, 20, 21, 22, 23, or 24 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved. In certain embodiments, the mismatch is specifically positioned within an oligonucleotide having a gapmer motif. In certain embodiments, the mismatch is at position 1, 2, 3, 4, 5, 6, 7, 8, 9, 10, or 11 from the 5′-end of the gap region. In certain embodiments, the mismatch is at position 1, 2, 3, 4, 5, or 6 from the 5′-end of the 5′ wing region or the 3′ wing region.

### B. SCN2A

In certain embodiments, oligomeric compounds comprise or consist of an oligonucleotide that is complementary to a target nucleic acid, wherein the target nucleic acid is an SCN2A nucleic acid. In certain embodiments, the SCN2A nucleic acid has the sequence set forth in SEQ ID NO: 1 (GENBANK Accession No. NM_001040142.2) or SEQ ID NO: 2 (GENBANK Accession No. NC_000002.12 truncated from nucleotides 165127001 to 165395000).

In certain embodiments, contacting a cell with an oligomeric compound complementary to SEQ ID NO: 1 or SEQ ID NO: 2 reduces the amount of SCN2A RNA in a cell, and in certain embodiments reduces the amount of SCN1A protein in a cell. In certain embodiments, contacting a cell with a modified oligonucleotide complementary to SEQ ID NO: 1 or SEQ ID NO: 2 reduces the amount of SCN2A RNA in a cell, and in certain embodiments reduces the amount of SCN2A protein in a cell. In certain embodiments, the cell is in vitro. In certain embodiments, the cell is in a subject. In certain embodiments, the oligomeric compound consists of a modified oligonucleotide. In certain embodiments, contacting a cell in a subject with an oligomeric compound complementary to SEQ ID NO: 1 or SEQ ID NO: 2 ameliorates one or more symptoms or hallmarks of a disease or disorder associated with a voltage-gated sodium channel protein. In certain embodiments, the voltage-gated sodium channel protein is SCN2A. In certain embodiments, the subject has a disease or disorder associated with a voltage-gated sodium channel protein that is not SCN2A In certain embodiments, the subject has a disease or disorder associated with SCN1A. In certain embodiments, the disease or disorder is a Developmental or Epileptic Encephalopathy, such as Early Seizure Onset Epileptic Encephalopathy, Late Seizure Onset Epileptic or Encephalopathy, Benign Familial Neonatal-Infantile Seizures; in certain embodiments, the disease or disorder is an intellectual disability or an autism spectrum disorder; in certain embodiments, the disease or disorder is Dravet Syndrome.

In certain embodiments, the symptom or hallmark is any of seizures, hypotonia, sensory issues, such as sensory integration disorders, motor dysfunctions, intellectual and cognitive dysfunctions, movement and balance dysfunctions, visual dysfunctions, delayed language and speech, gastrointestinal disorders (for example, gastroesophageal reflux, diarrhea, constipation, dysmotility, and the like), neurodevelopmental delays, sleep problems, sudden unexpected death in epilepsy, motor development delays, delayed social milestones, repetitive actions, uncoordinated oral movements. In certain embodiments, the seizures include focal, clonic, tonic, and generalized tonic and clonic seizures, prolonged seizures (often lasting longer than 10 minutes), and frequent seizures (for example, convulsive, myoclonic, absence, focal, obtundation status, and tonic seizures).

In certain embodiments, an oligomeric compound complementary to SEQ ID NO: 1 or SEQ ID NO: 2 is capable of reducing the detectable amount of SCN2A RNA in vitro by at least 10%, at least 20%, at least 30%, at least 40%, at least 50%, at least 60%, at least 70%, at least 80%, or at least 90% in the standard in vitro assay. In certain embodiments, an oligomeric compound complementary to SEQ ID NO: 1 or SEQ ID NO: 2 is capable of reducing the detectable amount of SCN2A protein in vitro by at least 10%, at least 20%, at least 30%, at least 40%, at least 50%, at least 60%, at least 70%, at least 80%, or at least 90% in the standard in vitro assay. In certain embodiments, an oligomeric compound complementary to SEQ ID NO: 1 or SEQ ID NO: 2 is capable of reducing the detectable amount of SCN2A RNA in vivo by at least 10%, at least 20%, at least 30%, at least 40%, at least 50%, at least 60%, at least 70%, at least 80%, or at least 90% when administered according to the standard in vivo assay. In certain embodiments, an oligomeric compound complementary to SEQ ID NO: 1 or SEQ ID NO: 2 is capable of reducing the detectable amount of SCN2A protein in vivo by at least 10%, at least 20%, at least 30%, at least 40%, at least 50%, at least 60%, at least 70%, at least 80%, or at least 90% when administered according to the standard in vivo assay. In certain embodiments, an oligomeric compound complementary to SEQ ID NO: 1 or SEQ ID NO: 2, is capable of reducing the detectable amount of SCN2A RNA in the CSF of a subject by at least 10%, at least 20%, at least 30%, at least 40%, at least 50%, at least 60%, at least 70%, at least 80%, or at least 90%. In certain embodiments, an oligomeric compound complementary to SEQ ID NO: 1 or SEQ ID NO: 2, is capable of reducing the detectable amount of SCN2A protein in the CSF of a subject by at least 10%, at least 20%, at least 30%, at least 40%, at least 50%, at least 60%, at least 70%, at is least 80%, or at least 90%.

In certain embodiments, oligomeric compounds do not comprise a bicyclic sugar moiety. In certain embodiments, oligomeric compounds do not comprise more than one, two, three, four, five, six, seven, eight, or nine bicyclic sugar moieties. In certain embodiments, oligomeric compounds comprise one or two wing segments that comprise a nucleoside that is not a bicyclic nucleoside. In certain embodiments, oligomeric compounds do not comprise a LNA sugar moiety. In certain embodiments, oligomeric compounds do not comprise more than one, two, three, four, five, six, seven, eight, or nine LNA sugar moieties. In certain embodiments, oligomeric compounds comprise one or two wing segments that comprise a nucleoside that is not a LNA nucleoside.

### C. Certain Target Nucleic Acids in Certain Tissues

In certain embodiments, oligomeric compounds comprise or consist of an oligonucleotide comprising a portion that is complementary to a target nucleic acid, wherein the target nucleic acid is expressed in a pharmacologically relevant tissue. In certain embodiments, the pharmacologically relevant tissues are the cells and tissues that comprise the central nervous system. Such tissues include brain, including but not limited to the cortex and the hippocampus, and the spinal cord.

### VI. Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid (“artificial CSF” or “aCSF”). In certain embodiments, the artificial cerebrospinal fluid is pharmaceutical grade.

In certain embodiments, a pharmaceutical composition comprises a modified oligonucleotide and artificial cerebrospinal fluid. In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to a subject, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents comprising an oligomeric compound provided herein to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), intraneural, perineural, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphate linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or salt thereof” expressly includes all such forms that may be fully or partially protonated/de-protonated/in association with a cation. In certain instances, one or more specific cation is identified.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in PBS. In certain embodiments, modified oligonucleotides or oligomeric compounds are in water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide or oligomeric compound. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid. For example, where a modified oligonucleotide or an oligomeric compound is in solution comprising sodium (e.g., saline), the modified oligonucleotide or oligomeric compound may be partially or fully de-protonated and in association with Na+ ions. However, the mass of the protons are nevertheless counted toward the weight of the dose, and the mass of the Na+ ions are not counted toward the weight of the dose. Thus, for example, a dose, or dosage unit, of 10 mg of Compound No. 1348259, equals the number of fully protonated molecules that weighs 10 mg. This would be equivalent to 10.59 mg of solvent-free, sodium acetate-free, anhydrous sodiated Compound No. 1348259. When an oligomeric compound comprises a conjugate group, the mass of the conjugate group is included in calculating the dose of such oligomeric compound. If the conjugate group also has an acid, the conjugate group is likewise assumed to be fully protonated for the purpose of calculating dose.

### VII. Certain Compositions

### 1. Compound No. 1348259

In certain embodiments, Compound No. 1348259 is characterized as a 5-10-5 MOE gapmer having a sequence (from 5′ to 3′) of GCATAATCCCATTATACAAA (SEQ ID NO: 2493), wherein each of nucleosides 1-5 and 16-20 (from 5′ to 3′) are 2′-MOE nucleosides and each of nucleosides 6-15 are 2′-β-D-deoxynucleosides, wherein the internucleoside linkages between nucleosides 2 to 3, 3 to 4, 4 to 5, 5 to 6, 16 to 17, and 17 to 18 are phosphodiester internucleoside linkages, the internucleoside linkages between nucleosides 1 to 2, 6 to 7, 7 to 8, 8 to 9, 9 to 10, 10 to 11, 11 to 12, 12 to 13, 13 to 14, 14 to 15, 15 to 16, 18 to 19, and 19 to 20 are phosphorothioate internucleoside linkages, and wherein each cytosine is a 5-methyl cytosine.

In certain embodiments, Compound No. 1348259 is represented by the following chemical notation: Gₑₛ^{m}CₑₒAₑₒTₑₒA_{ds}T_{ds}^{m}C_{ds}^{m}C_{ds}A_{ds}T_{ds}T_{ds}A_{ds}T_{ds}Aₑₒ^{m}CₑₒAₑₛAₑₛAₑ (SEQ ID NO: 2493), wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

In certain embodiments, Compound No. 1348259 is represented by the following chemical structure:

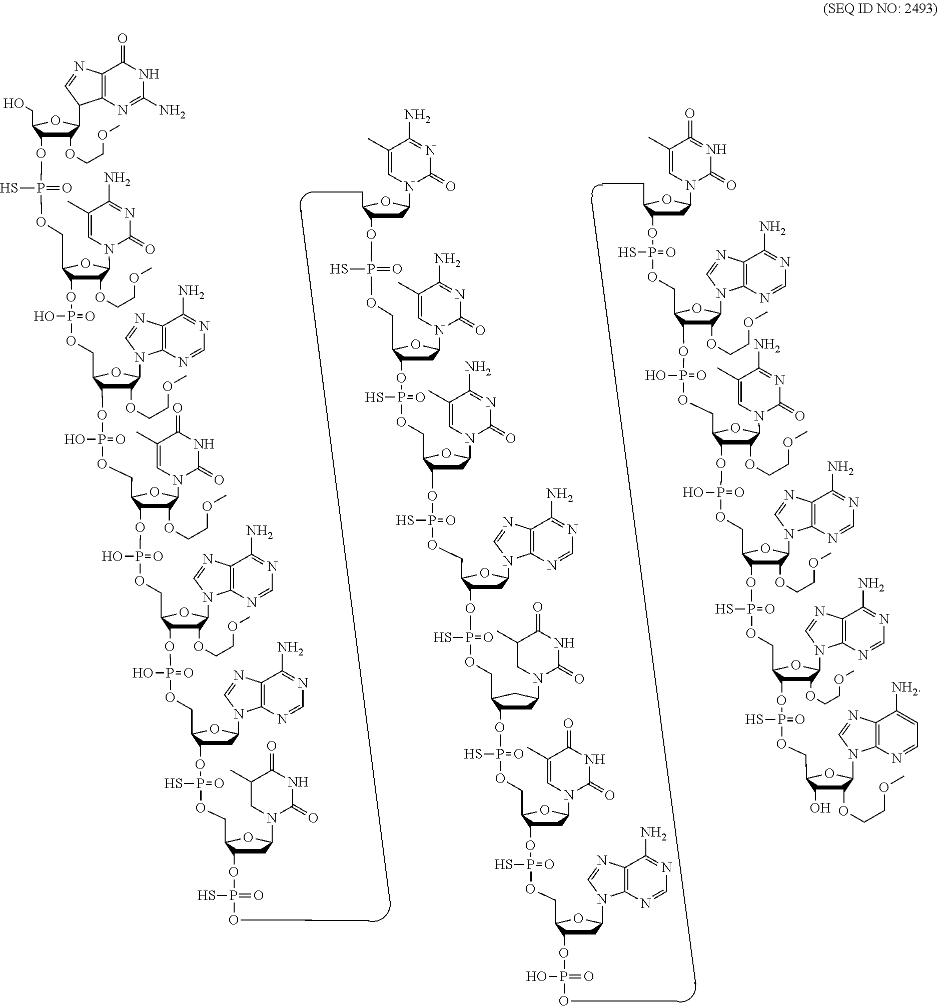

### Structure 1. Compound No. 1348259

In certain embodiments, the sodium salt of Compound No. 1348259 is represented by the following chemical structure:

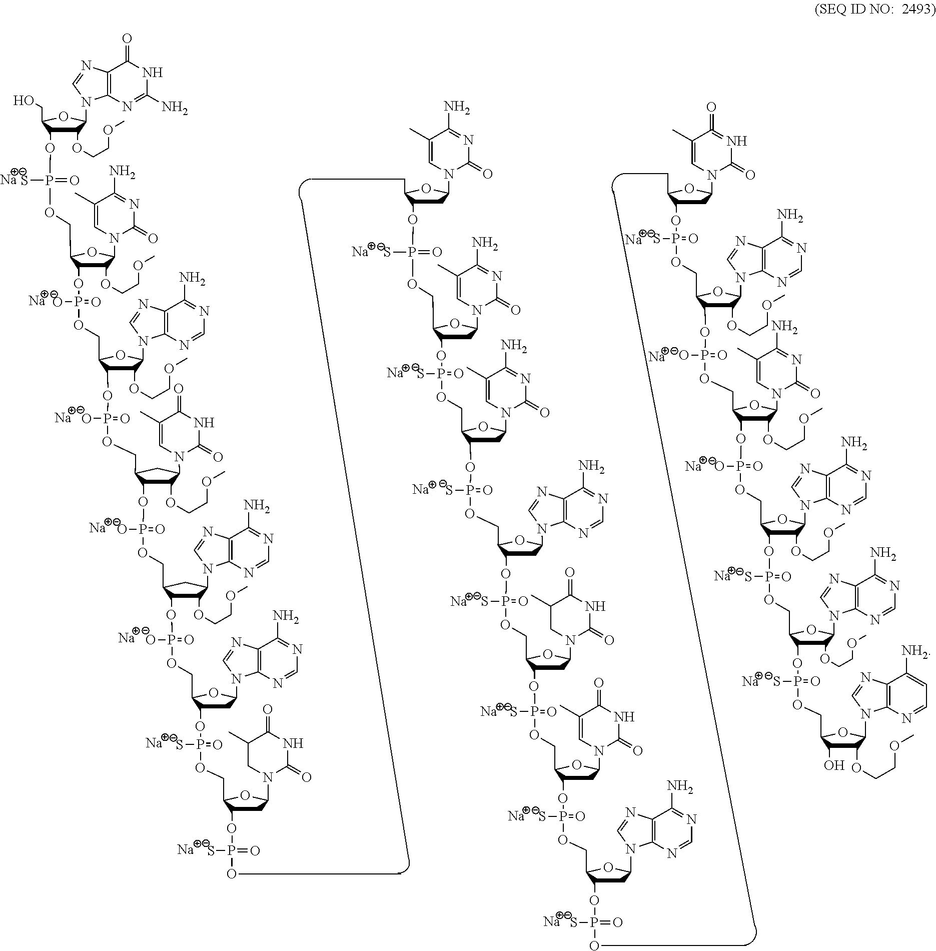

### Structure 2. The Sodium Salt of Compound No. 1348259

### 2. Compound No. 1348289

In certain embodiments, Compound No. 1348289 is characterized as a 6-10-4 MOE gapmer having a sequence (from 5′ to 3′) of CACGACATATTTTTCTACAC (SEQ ID NO: 2514), wherein each of nucleosides 1-6 and 17-20 (from 5′ to 3′) are 2′-MOE nucleosides and each of nucleosides 7-16 are 2′-β-D-deoxynucleosides, wherein the internucleoside linkages between nucleosides 2 to 3, 3 to 4, 4 to 5, 5 to 6, 6 to 7, and 17 to 18 are phosphodiester internucleoside linkages, the internucleoside linkages between nucleosides 1 to 2, 7 to 8, 8 to 9, 9 to 10, 10 to 11, 11 to 12, 12 to 13, 13 to 14, 14 to 15, 15 to 16, 16 to 17, 18 to 19, and 19 to 20 are phosphorothioate internucleoside linkages, and wherein each cytosine is a 5-methyl cytosine.

In certain embodiments, Compound No. 1348289 is represented by the following chemical notation: ^{m}CₑₛAₑₒ^{m}CₑₒGₑₒAₑₒ^{m}CₑₒA_{ds}T_{ds}A_{ds}T_{ds}T_{ds}T_{ds}T_{ds}T_{ds}^{m}C_{ds}T_{ds}Aₑₒ^{m}CₑₛAₑₛ^{m}Cₑ (SEQ ID NO: 2514), wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

In certain embodiments, Compound No. 1348289 is represented by the following chemical structure:

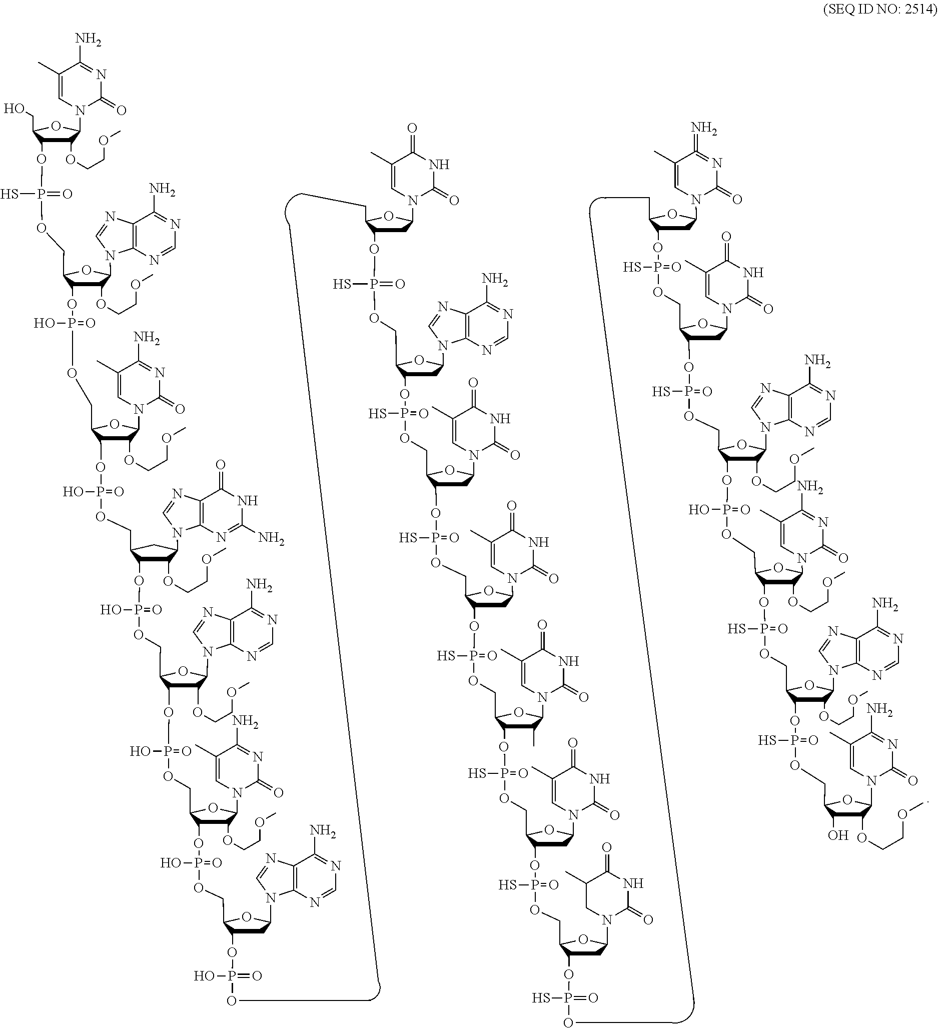

### Structure 3. Compound No. 1348289

In certain embodiments, the sodium salt of Compound No. 1348289 is represented by the following chemical structure:

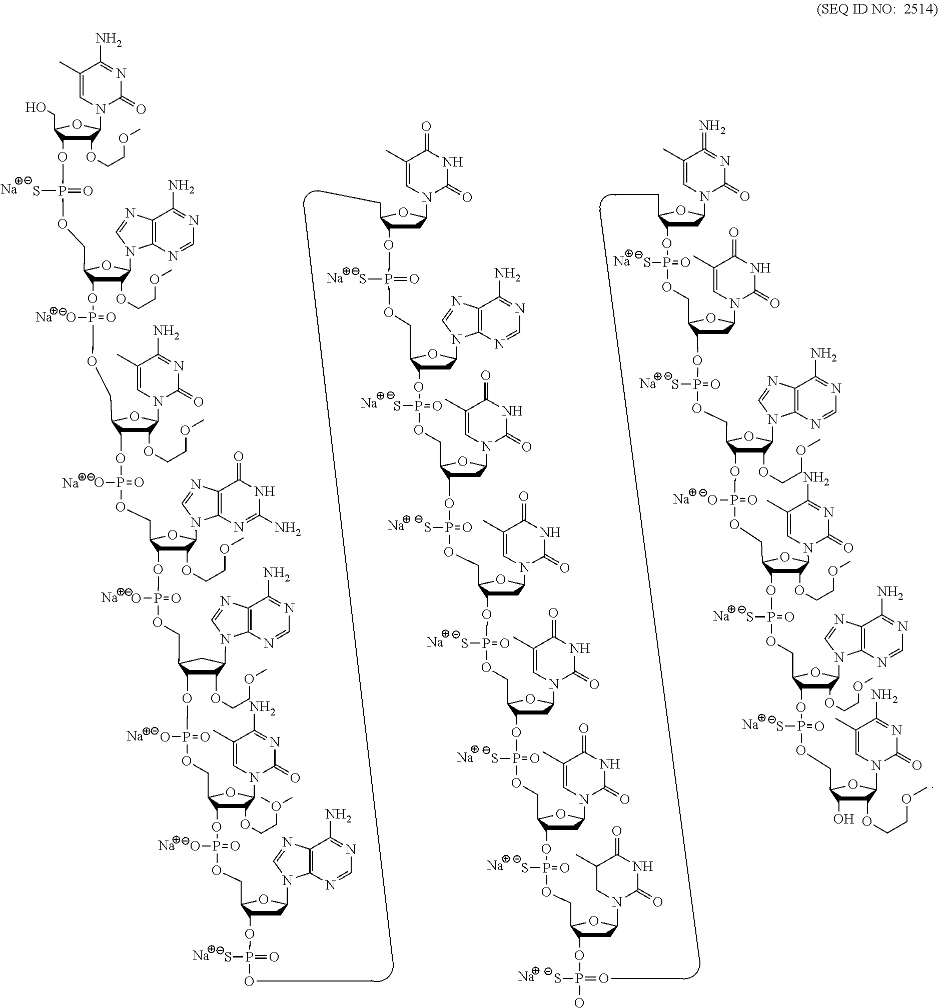

Structure 4. The sodium Salt of Compound No. 1348289

### 3. Compound No. 1348290

In certain embodiments, Compound No. 1348290 is characterized as a 6-10-4 MOE gapmer having a sequence (from 5′ to 3′) of CCACGACATATTTTTCTACA (SEQ ID NO: 2510), wherein each of nucleosides 1-6 and 17-20 (from 5′ to 3′) are 2′-MOE nucleosides and each of nucleosides 7-16 are 2′-β-D-deoxynucleosides, wherein the internucleoside linkages between nucleosides 2 to 3, 3 to 4, 4 to 5, 5 to 6, 6 to 7, and 17 to 18 are phosphodiester internucleoside linkages, the internucleoside linkages between nucleosides 1 to 2, 7 to 8, 8 to 9, 9 to 10, 10 to 11, 11 to 12, 12 to 13, 13 to 14, 14 to 15, 15 to 16, 16 to 17, 18 to 19, and 19 to 20 are phosphorothioate internucleoside linkages, and wherein each cytosine is a 5-methyl cytosine.

In certain embodiments, Compound No. 1348290 is represented by the following chemical notation: ^{m}CₑₛCₑₒAₑₒ^{m}CₑₒGₑₒAₑₒ^{m}C_{ds}A_{ds}T_{ds}A_{ds}T_{ds}T_{ds}T_{ds}T_{ds}T_{ds}^{m}C_{ds}TₑₒAₑₛ^{m}CₑₛAₑ (SEQ ID NO: 2510), wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

In certain embodiments, Compound No. 1348290 is represented by the following chemical structure:

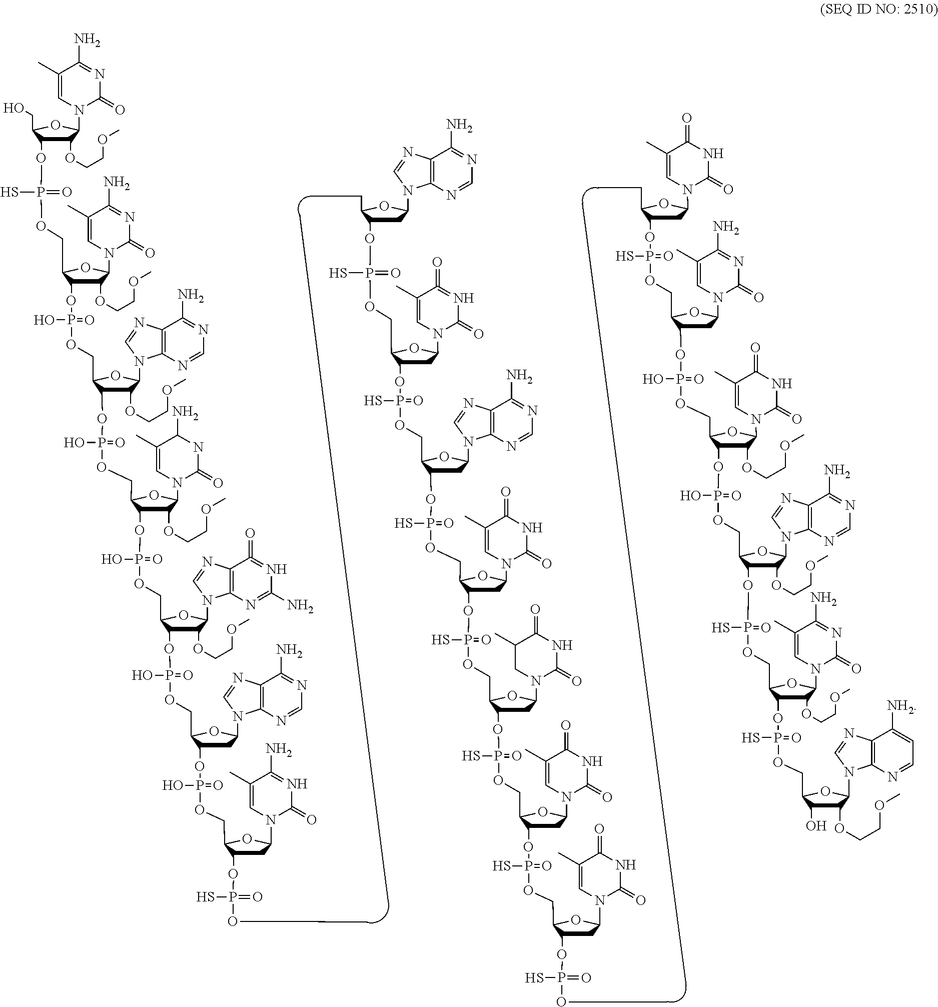

### Structure 5: Compound No. 1348290

In certain embodiments, the sodium salt of Compound No. 1348290 is represented by the following chemical structure:

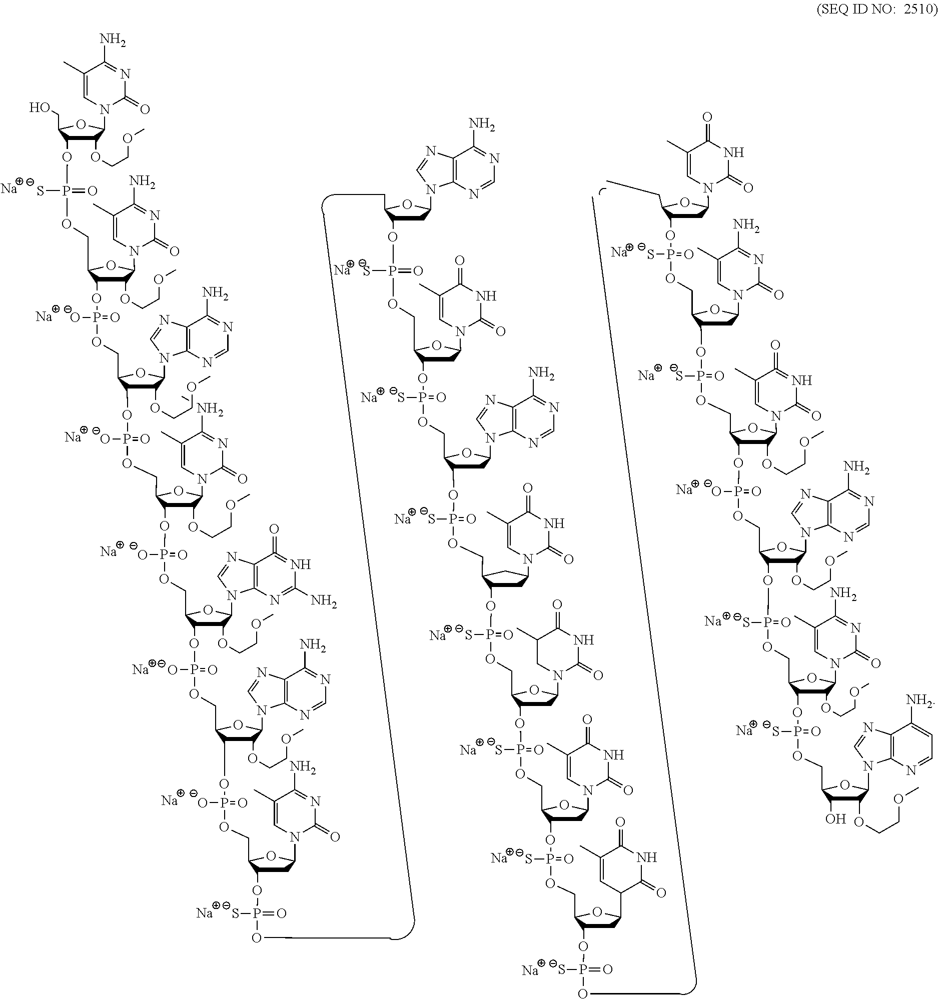

### Structure 6: The Sodium Salt of Compound No. 1348290

### 4. Compound No. 1348331

In certain embodiments, Compound No. 1348331 is characterized as a 6-10-4 MOE gapmer having a sequence (from 5′ to 3′) of TCTGCATGTAACCTTTATAC (SEQ ID NO: 2487), wherein each of nucleosides 1-6 and 17-20 (from 5′ to 3′) are 2′-MOE nucleosides and each of nucleosides 7-16 are 2′-β-D-deoxynucleosides, wherein the internucleoside linkages between nucleosides 2 to 3, 3 to 4, 4 to 5, 5 to 6, 6 to 7, and 17 to 18 are phosphodiester internucleoside linkages, the internucleoside linkages between nucleosides 1 to 2, 7 to 8, 8 to 9, 9 to 10, 10 to 11, 11 to 12, 12 to 13, 13 to 14, 14 to 15, 15 to 16, 16 to 17, 18 to 19, and 19 to 20 are phosphorothioate internucleoside linkages, and wherein each cytosine is a 5-methyl cytosine.

In certain embodiments, Compound No. 1348331 is represented by the following chemical notation: Tₑₛ^{m}CₑₒTₑₒGₑₒ^{m}CₑₒAₑₒT_{ds}G_{ds}T_{ds}A_{ds}A_{ds}^{m}C_{ds}^{m}C_{ds}T_{ds}T_{ds}T_{ds}AₑₒTₑₛAₑₛ^{m}Cₑ (SEQ ID NO: 2487), wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

In certain embodiments, Compound No. 1348331 is represented by the following chemical structure:

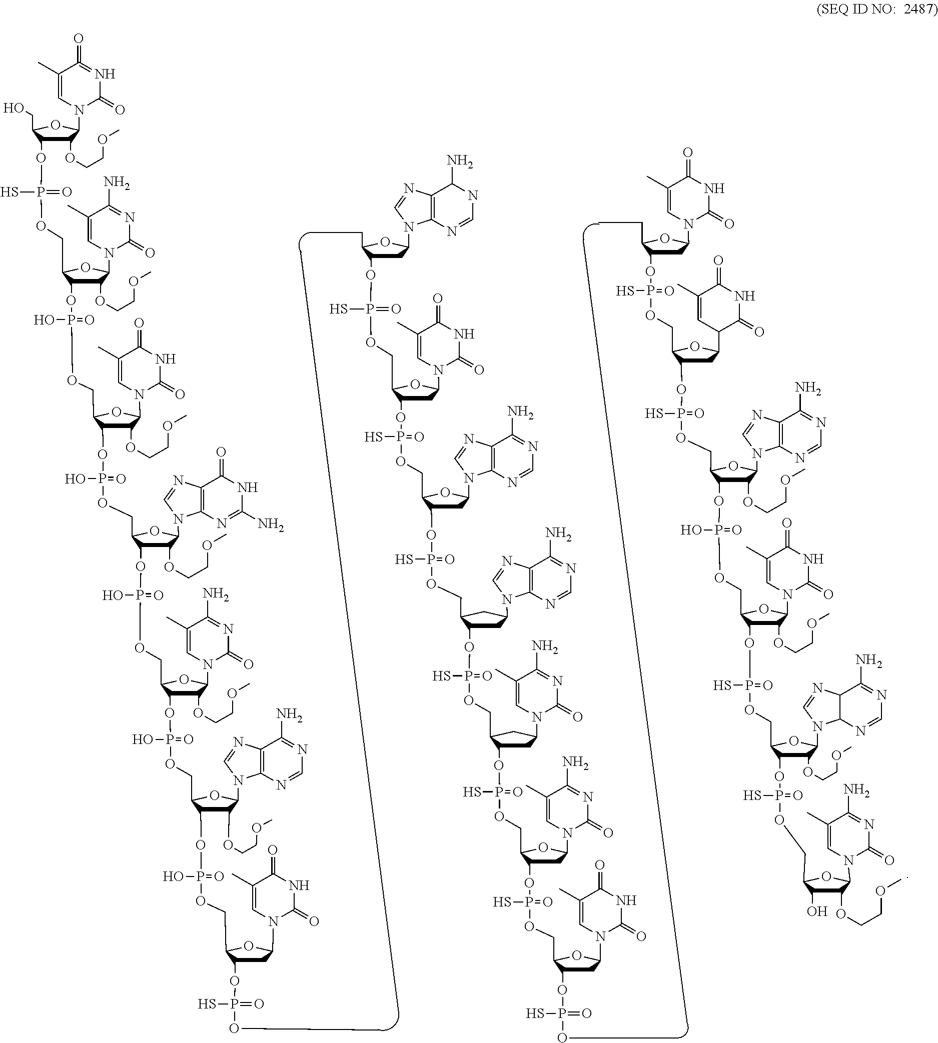

### Structure 7: Compound No. 1348331

In certain embodiments, the sodium salt of Compound No. 1348331 is represented by the following chemical structure:

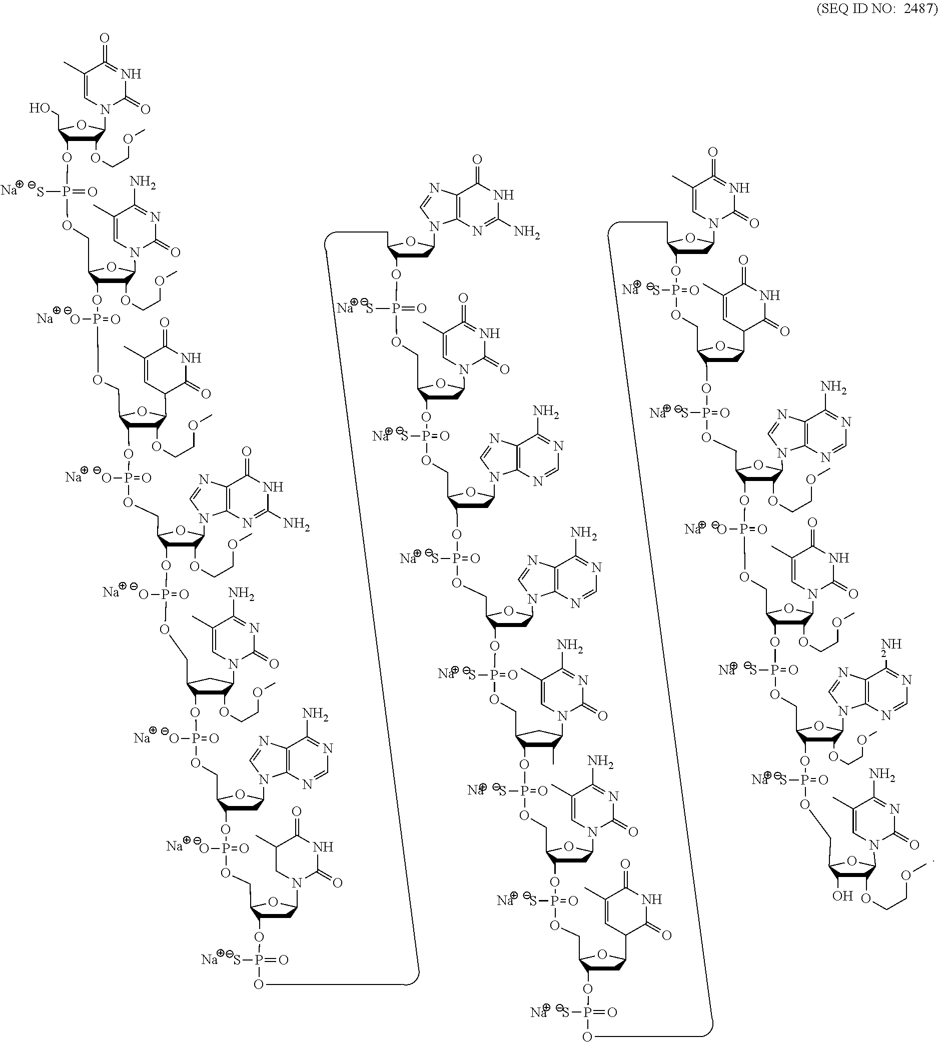

### Structure 8: The Sodium Salt of Compound No. 1348331

### 5. Compound No. 1348347

In certain embodiments, Compound No. 1348347 is characterized as a 6-10-4 MOE gapmer having a sequence (from 5′ to 3′) of GCATAATCCCATTATACAAA (SEQ ID NO: 2493), wherein each of nucleosides 1-6 and 17-20 (from 5′ to 3′) are 2′-MOE nucleosides and each of nucleosides 7-16 are 2′-β-D-deoxynucleosides, wherein the internucleoside linkages between nucleosides 2 to 3, 3 to 4, 4 to 5, 5 to 6, 6 to 7, and 17 to 18 are phosphodiester internucleoside linkages, the internucleoside linkages between nucleosides 1 to 2, 7 to 8, 8 to 9, 9 to 10, 10 to 11, 11 to 12, 12 to 13, 13 to 14, 14 to 15, 15 to 16, 16 to 17, 18 to 19, and 19 to 20 are phosphorothioate internucleoside linkages, and wherein each cytosine is a 5-methyl cytosine.

In certain embodiments, Compound No. 1348347 is represented by the following chemical notation: Gₑₛ^{m}CₑₒAₑₒTₑₒAₑₒAₑₒT_{ds}^{m}C_{ds}^{m}C_{ds}A_{ds}T_{ds}T_{ds}A_{ds}T_{ds}A_{ds}^{m}CₑₒAₑₛAₑₛAₑ (SEQ ID NO: 2493), wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

In certain embodiments, Compound No. 1348347 is represented by the following chemical structure:

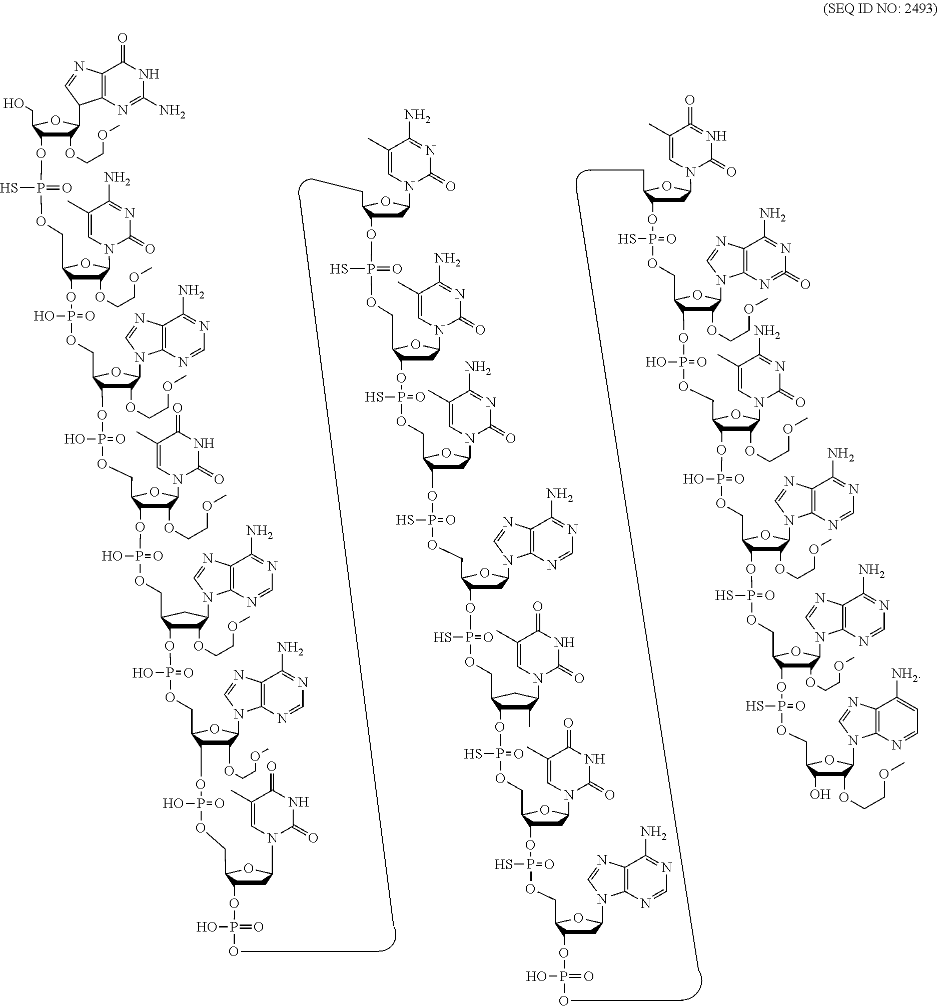

### Structure 9: Compound No. 1348347

In certain embodiments, the sodium salt of Compound No. 1348347 is represented by the following chemical structure:

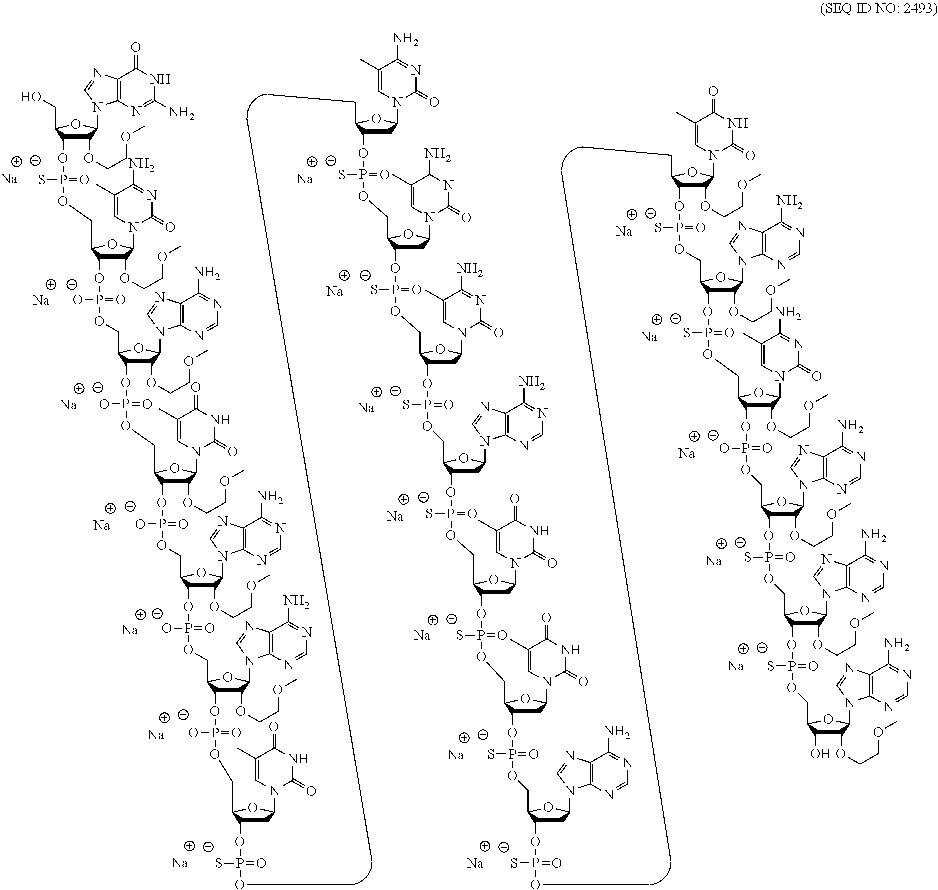

### Structure 10: The Sodium Salt of Compound No. 1348347

### 6. Compound No. 1348937

In certain embodiments, Compound No. 1348937 is characterized as a 5-8-5 MOE gapmer having a sequence (from 5′ to 3′) of CTGCATGTAACCTITATA (SEQ ID NO: 2534), wherein each of nucleosides 1-5 and 14-18 (from 5′ to 3′) are 2′-MOE nucleosides and each of nucleosides 6-13 are 2′-β-D-deoxynucleosides, wherein the internucleoside linkages between nucleosides 2 to 3, 3 to 4, 4 to 5, 14 to 15 and 15 to 16 are phosphodiester internucleoside linkages, the internucleoside linkages between nucleosides 1 to 2, 5 to 6, 6 to 7, 7 to 8, 8 to 9, 9 to 10, 10 to 11, 11 to 12, 12 to 13, 13 to 14, 16 to 17, and 17 to 18 are phosphorothioate internucleoside linkages, and wherein each cytosine is a 5-methyl cytosine.

In certain embodiments, Compound No. 1348937 is represented by the following chemical notation: ^{m}CₑₛTₑₒGₑₒ^{m}CₑₒAₑₛT_{ds}G_{ds}A_{ds}A_{ds}^{m}C_{ds}^{m}C_{ds}T_{ds}TₑₒTₑₒAₑₛTₑₛAₑ (SEQ ID NO: 2534), wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

In certain embodiments, Compound No. 1348937 is represented by the following chemical structure:

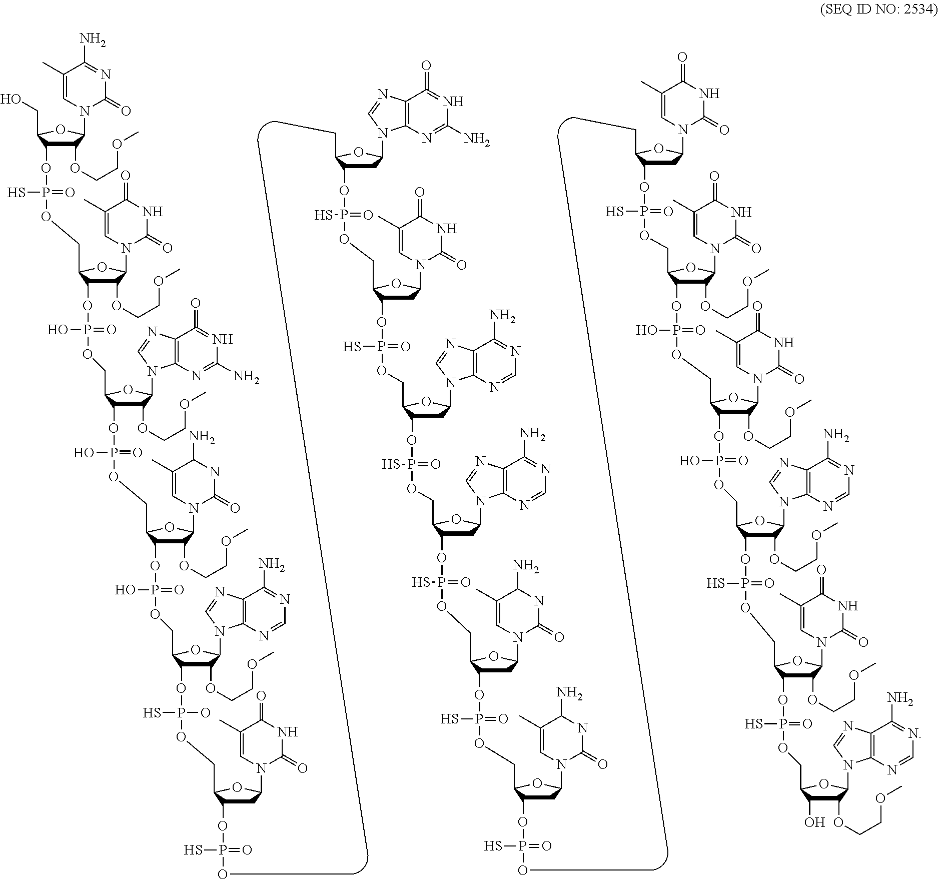

### Structure 11: Compound No. 1348937

In certain embodiments, the sodium salt of Compound No. 1348937 is represented by the following chemical structure:

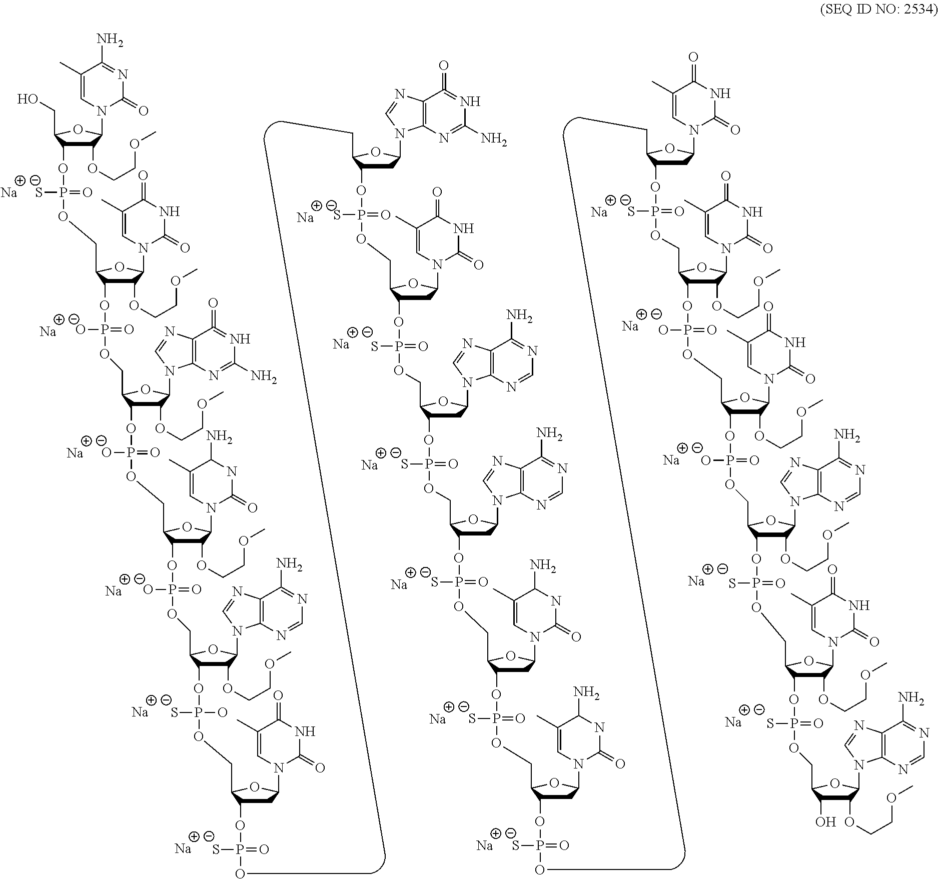

### Structure 12: The Sodium Salt of Compound No. 1348937

### VIII. Certain Hotspot Regions

In certain embodiments, nucleobases in the ranges specified below comprise a hotspot region of SCN2A nucleic acid. In certain embodiments, modified oligonucleotides that are complementary to an equal length portion within a hotspot region of SCN2A nucleic acid achieve an average of 69.9% or greater reduction of SCN2A RNA in vitro in the standard in vitro assay. In certain embodiments, modified oligonucleotides that are complementary to an equal length portion within a hotspot region of SCN2A nucleic acid achieve an average of 59% or greater reduction of SCN2A RNA in vivo in the standard in vivo assay.

### 1. Nucleobases 2306-2367 of SEQ ID NO: 1 or 199863-199905 of SEQ ID NO: 2

In certain embodiments, nucleobases 2306-2367 of SEQ ID NO: 1 or 199863-199905 of SEQ ID NO: 2 comprise a hotspot region. In certain embodiments, modified oligonucleotides are complementary to an equal length portion within nucleobases 2306-2367 of SEQ ID NO: 1 or 199863-199905 of SEQ ID NO: 2. In certain embodiments, modified oligonucleotides are 20 nucleobases in length. In certain embodiments, modified oligonucleotides are 18 nucleobases in length. In certain embodiments, modified oligonucleotides are 16, 17, 18, 19, 20, 21, or 22 nucleobases in length. In certain embodiments, modified oligonucleotides consist of 17-19 or 21-30 linked nucleosides. In certain embodiments, modified oligonucleotides are gapmers.

In certain embodiments, the gapmers are 5-10-5 MOE gapmers. In certain embodiments, the gapmers are 6-10-4 MOE gapmers. In certain embodiments, the gapmers are 4-10-6 MOE gapmers. In certain embodiments, the gapmers are 4-8-6 MOE gapmers. In certain embodiments, the gapmers are 6-8-4 MOE gapmers. In certain embodiments, the is gapmers are 5-8-5 MOE gapmers. In certain embodiments, the gapmers have the sugar motif in order from 5′ to 3′: eeeeeddddddddddeeeee, eeeeeeddddddddddeeee, eeeeddddddddddeeeeee, eeeeddddddddeeeeee, eeeeeeddddddddeeee, or eeeeeddddddddeeeee; wherein ‘d’ represents a 2′-β-D-deoxyribosyl sugar moiety, and ‘e’ represents a 2′-MOE sugar moiety. In certain embodiments, the gapmers comprise a 2′-substituted nucleoside in the gap. In certain embodiments, the 2′-substituted nucleoside comprises a 2′-OMe sugar moiety. In certain embodiments, the 2′-substituted nucleoside is at position 2 of the gap (5′ to 3′).

In certain embodiments, modified oligonucleotides do not comprise a bicyclic sugar moiety. In certain embodiments, modified oligonucleotides do not comprise more than one, two, three, four, five, six, seven, eight, or nine bicyclic sugar moieties. In certain embodiments, modified oligonucleotides comprise one or two wing segments that comprise a nucleoside that is not a bicyclic nucleoside. In certain embodiments, modified oligonucleotides do not comprise a LNA sugar moiety. In certain embodiments, modified oligonucleotides do not comprise more than one, two, three, four, five, six, seven, eight, or nine LNA sugar moieties. In certain embodiments, modified oligonucleotides comprise one or two wing segments that comprise a nucleoside that is not a LNA nucleoside.

In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in order from 5′ to 3′. In certain embodiments, the modified nucleotides have an internucleoside linkage motif of (from 5′ to 3′) of soooossssssssssooss, sooooossssssssssoss, sooossssssssssoooss, soosssssssssoooss, soooosssssssssoss, or sooosssssssssooss wherein each “s” represents a phosphorothioate internucleoside linkage and each “o” represents a phosphodiester internucleoside linkage.

The nucleobase sequences of SEQ ID NOs: 336, 488, 2021, 2097, 2174, 2250, 2326, 2403, 2499, 2500, 2501, 2502, and 2526 are complementary to an equal length portion within nucleobases 2306-2367 of SEQ ID NO: 1 or 199863-199905 of SEQ ID NO: 2.

The nucleobase sequence of Compound IDs: 909979, 1248427, 1248428, 1248429, 1248430, 1248431, 1248432, 1248433, 1348279, 1348282, 1348286, 1348297, 1348328, 1348343, 1348358, 1348360, 1348361, 1348362, 1348364, 1348365, 1348366, 1348367, is 1348378, and 1348380 are complementary to an equal length portion within nucleobases 2306-2367 of SEQ ID NO: 1 or 199863-199905 of SEQ ID NO: 2.

In certain embodiments, modified oligonucleotides complementary to an equal length portion within nucleobases 2306-2367 of SEQ ID NO: 1 or 199863-199905 of SEQ ID NO: 2 achieve at least 53% reduction of SCN2A RNA in vitro in the standard in vitro assay. In certain embodiments, modified oligonucleotides complementary to an equal length portion within nucleobases 2306-2367 of SEQ ID NO: 1 or 199863-199905 of SEQ ID NO: 2 achieve an average of 69.9% reduction of SCN2A RNA in vitro in the standard in vitro assay. In certain embodiments, modified oligonucleotides complementary to an equal length portion within nucleobases 2306-2367 of SEQ ID NO: 1 or 199863-199905 of SEQ ID NO: 2 achieve an average of 77.1% reduction of SCN2A RNA in vivo in the standard in vivo assay. In certain embodiments, modified oligonucleotides complementary to an equal length portion within nucleobases 2306-2367 of SEQ ID NO: 1 or 199863-199905 of SEQ ID NO: 2 achieve an average of 63.2% reduction of SCN2A RNA in vivo in the standard in vivo assay.

### 2. Nucleobases 3499-3557 of SEQ ID NO: 1 or 227493-227551 of SEQ ID NO: 2

In certain embodiments, nucleobases 3499-3557 of SEQ ID NO: 1 or 227493-227551 of SEQ ID NO: 2 comprise a hotspot region. In certain embodiments, modified oligonucleotides are complementary to an equal length portion within nucleobases 3499-3557 of SEQ ID NO: 1 or 227493-227551 of SEQ ID NO: 2. In certain embodiments, modified oligonucleotides are 20 nucleobases in length. In certain embodiments, modified oligonucleotides are 18 nucleobases in length. In certain embodiments, modified oligonucleotides are 16, 17, 18, 19, 20, 21, or 22 nucleobases in length. In certain embodiments, modified oligonucleotides consist of 17-19 or 21-30 linked nucleosides. In certain embodiments, modified oligonucleotides are gapmers.

In certain embodiments, the gapmers are 5-10-5 MOE gapmers. In certain embodiments, the gapmers are 6-10-4 MOE gapmers. In certain embodiments, the gapmers are 4-10-6 MOE gapmers. In certain embodiments, the gapmers are 4-8-6 MOE gapmers. In certain embodiments, the gapmers are 6-8-4 MOE gapmers. In certain embodiments, the gapmers are 5-8-5 MOE gapmers. In certain embodiments, the gapmers have the sugar motif in order from 5′ to 3′: eeeeeddddddddddeeeee, eeeeeeddddddddddeeee, eeeeddddddddddeeeeee, eeeeddddddddeeeeee, eeeeeeddddddddeeee, or eeeeeddddddddeeeee; wherein ‘d’ represents a 2′-β-D-deoxyribosyl sugar moiety, and ‘e’ represents a 2′-MOE sugar moiety. In certain embodiments, the gapmers comprise a 2′-substituted nucleoside in the gap. In certain embodiments, the 2′-substituted nucleoside comprises a 2′-OMe sugar moiety. In certain embodiments, the 2′-substituted nucleoside is at position 2 of the gap (5′ to 3′).

In certain embodiments, modified oligonucleotides do not comprise a bicyclic sugar moiety. In certain embodiments, modified oligonucleotides do not comprise more than one, two, three, four, five, six, seven, eight, or nine bicyclic sugar moieties. In certain embodiments, modified oligonucleotides comprise one or two wing segments that comprise a nucleoside that is not a bicyclic nucleoside. In certain embodiments, modified oligonucleotides do not comprise a LNA sugar moiety. In certain embodiments, modified oligonucleotides do not comprise more than one, two, three, four, five, six, seven, eight, or nine LNA sugar moieties. In certain embodiments, modified oligonucleotides comprise one or two wing segments that comprise a nucleoside that is not a LNA nucleoside.

In certain embodiments, the internucleoside linkages of the modified oligonucleotides are phosphorothioate internucleoside linkages and phosphodiester internucleoside linkages.

In certain embodiments, the phosphodiester (“o”) and phosphorothioate (“s”) internucleoside linkages are arranged in order from 5′ to 3′. In certain embodiments, the modified nucleotides have an internucleoside linkage motif of (from 5′ to 3′) of soooossssssssssooss, sooooossssssssssoss, sooossssssssssoooss, soosssssssssoooss, soooosssssssssoss, or sooosssssssssooss wherein each “s” represents a phosphorothioate internucleoside linkage and each “o” represents a phosphodiester internucleoside linkage.

The nucleobase sequences of SEQ ID NOs: 181, 259, 643, 720, 796, 2504, 2505, 2506, 2507, 2508, 2509, 2510, 2511, 2512, 2513, 2514, and 2521 are complementary to an equal length portion within nucleobases 3499-3557 of SEQ ID NO: 1 or 227493-227551 of SEQ ID NO: 2.

The nucleobase sequence of Compound IDs: 909989, 909990, 1248487, 1248488, 1248489, 1348289, 1348290, 1348291, 1348292, 1348295, 1348298, 1348302, 1348303, 1348304, 1348306, 1348307, 1348369, 1348370, 1348371, 1348373, 1348374, 1348375, 1348376, 1348377, 1348381, 1348382, 1348383, 1348384, 1348385, 1348386, 1348387, 1348405, 1348411, 1348423, 1348439, 1348440, 1348441, 1348442, 1348443, 1348444, 1348446, 1348447, and 1348456 are complementary to an equal length portion within nucleobases 3499-3557 of SEQ ID NO: 1 or 227493-227551 of SEQ ID NO: 2.

In certain embodiments, modified oligonucleotides complementary to an equal length portion within nucleobases 3499-3557 of SEQ ID NO: 1 or 227493-227551 of SEQ ID NO: 2 achieve at least 75% reduction of SCN2A RNA in vitro in the standard in vitro assay. In certain embodiments, modified oligonucleotides complementary to an equal length portion within nucleobases 3499-3557 of SEQ ID NO: 1 or 227493-227551 of SEQ ID NO: 2 achieve an average of 81.6% reduction of SCN2A RNA in vitro in the standard in vitro assay. In certain embodiments, modified oligonucleotides complementary to an equal length portion within nucleobases 3499-3557 of SEQ ID NO: 1 or 227493-227551 of SEQ ID NO: 2 achieve an average of 76.6% reduction of SCN2A RNA in vivo in the standard in vivo assay. In certain embodiments, modified oligonucleotides complementary to an equal length portion within nucleobases 3499-3557 of SEQ ID NO: 1 or 227493-227551 of SEQ ID NO: 2 achieve an average of 67.2% reduction of SCN2A RNA in vivo in the standard in vivo assay.

### 3. Nucleobases 243124-243204 of SEQ ID NO: 2

In certain embodiments, nucleobases 243124-243204 of SEQ ID NO: 2 comprise a hotspot region. In certain embodiments, modified oligonucleotides are complementary to an equal length portion within nucleobases 243124-243204 of SEQ ID NO: 2. In certain embodiments, modified oligonucleotides are 20 nucleobases in length. In certain embodiments, modified oligonucleotides are 18 nucleobases in length. In certain embodiments, modified oligonucleotides are 16, 17, 18, 19, 20, 21, or 22 nucleobases in length. In certain embodiments, modified oligonucleotides consist of 17-19 or 21-30 linked nucleosides. In certain embodiments, modified oligonucleotides are gapmers.

In certain embodiments, the gapmers are 5-10-5 MOE gapmers. In certain embodiments, the gapmers are 6-10-4 MOE gapmers. In certain embodiments, the gapmers are 4-10-6 MOE gapmers. In certain embodiments, the gapmers are 4-8-6 MOE gapmers. In certain embodiments, the gapmers are 6-8-4 MOE gapmers. In certain embodiments, the gapmers are 5-8-5 MOE gapmers. In certain embodiments, the gapmers have the sugar motif in order from 5′ to 3′: eeeeeddddddddddeeeee, eeeeeeddddddddddeeee, eeeeddddddddddeeeeee, eeeeddddddddeeeeee, eeeeeeddddddddeeee, or eeeeeddddddddeeeee; wherein ‘d’ represents a 2′-β-D-deoxyribosyl sugar moiety, and ‘e’ represents a 2′-MOE sugar moiety. In certain embodiments, the gapmers comprise a 2′-substituted nucleoside in the gap. In certain embodiments, the 2′-substituted nucleoside comprises a 2′-OMe sugar moiety. In certain embodiments, the 2′-substituted nucleoside is at position 2 of the gap (5′ to 3′).

In certain embodiments, modified oligonucleotides do not comprise a bicyclic sugar moiety. In certain embodiments, modified oligonucleotides do not comprise more than one, two, three, four, five, six, seven, eight, or nine bicyclic sugar moieties. In certain embodiments, modified oligonucleotides comprise one or two wing segments that comprise a nucleoside that is not a bicyclic nucleoside. In certain embodiments, modified oligonucleotides do not comprise a LNA sugar moiety. In certain embodiments, modified oligonucleotides do not comprise more than one, two, three, four, five, six, seven, eight, or nine LNA sugar moieties. In certain embodiments, modified oligonucleotides comprise one or two wing segments that comprise a nucleoside that is not a LNA nucleoside.

In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in order from 5′ to 3′. In certain embodiments, the modified nucleotides have an internucleoside linkage motif of (from 5′ to 3′) of soooossssssssssooss, sooooossssssssssoss, sooossssssssssoooss, soosssssssssoooss, soooosssssssssoss, or sooosssssssssooss wherein each “s” represents a phosphorothioate internucleoside linkage and each “o” represents a phosphodiester internucleoside linkage.

The nucleobase sequences of SEQ ID NOs: 491, 567, 644, 721, 797, 2177, 2253, 2315, 2329, 2406, and 2527 are complementary to an equal length portion within nucleobases 243124-243204 of SEQ ID NO: 2.

The nucleobase sequence of Compound IDs: 1248507, 1248508, 1248509, 1248510, 1248511, 1248512, 1248513, 1248514, 1248515, 1250138, 1348299, 1348379, 1348388, and 1348397 are complementary to an equal length portion within nucleobases 243124-243204 of SEQ ID NO: 2.

In certain embodiments, modified oligonucleotides complementary to an equal length portion within nucleobases 243124-243204 of SEQ ID NO: 2 achieve at least 51% reduction of SCN2A RNA in vitro in the standard in vitro assay. In certain embodiments, modified oligonucleotides complementary to an equal length portion within nucleobases 243124-243204 of SEQ ID NO: 2 achieve an average of 71.4% reduction of SCN2A RNA in vitro in the standard in vitro assay. In certain embodiments, modified oligonucleotides complementary to an equal length portion within nucleobases 243124-243204 of SEQ ID NO: 2 achieve an average of 61.3% reduction of SCN2A RNA in vivo in the standard in vivo assay. In certain embodiments, modified oligonucleotides complementary to an equal length portion within nucleobases 243124-243204 of SEQ ID NO: 2 achieve an average of 61.5% reduction of SCN2A RNA in vivo in the standard in vivo assay.

### 4. Nucleobases 243917-244073 of SEQ ID NO: 2

In certain embodiments, nucleobases 243917-244073 of SEQ ID NO: 2 comprise a hotspot region. In certain embodiments, modified oligonucleotides are complementary to an equal length portion within nucleobases 243917-244073 of SEQ ID NO: 2. In certain embodiments, modified oligonucleotides are 20 nucleobases in length. In certain embodiments, modified oligonucleotides are 18 nucleobases in length. In certain embodiments, modified oligonucleotides are 16, 17, 18, 19, 20, 21, or 22 nucleobases in length. In certain embodiments, modified oligonucleotides consist of 17-19 or 21-30 linked nucleosides. In certain embodiments, modified oligonucleotides are gapmers.

In certain embodiments, the gapmers are 5-10-5 MOE gapmers. In certain embodiments, the gapmers are 6-10-4 MOE gapmers. In certain embodiments, the gapmers are 4-10-6 MOE gapmers. In certain embodiments, the gapmers are 4-8-6 MOE gapmers. In certain embodiments, the gapmers are 6-8-4 MOE gapmers. In certain embodiments, the gapmers are 5-8-5 MOE gapmers. In certain embodiments, the gapmers have the sugar motif in order from 5′ to 3′: eeeeeddddddddddeeeee, eeeeeeddddddddddeeee, eeeeddddddddddeeeeee, eeeeddddddddeeeeee, eeeeeeddddddddeeee, or eeeeeddddddddeeeee; wherein ‘d’ represents a 2′-β-D-deoxyribosyl sugar moiety, and ‘e’ represents a 2′-MOE sugar moiety. In certain embodiments, the gapmers comprise a 2′-substituted nucleoside in the gap. In certain embodiments, the 2′-substituted nucleoside comprises a 2′-OMe sugar moiety. In certain embodiments, the 2′-substituted nucleoside is at position 2 of the gap (5′ to 3′).

In certain embodiments, modified oligonucleotides do not comprise a bicyclic sugar moiety. In certain embodiments, modified oligonucleotides do not comprise more than one, two, three, four, five, six, seven, eight, or nine bicyclic sugar moieties. In certain embodiments, modified oligonucleotides comprise one or two wing segments that comprise a nucleoside that is not a bicyclic nucleoside. In certain embodiments, modified oligonucleotides do not comprise a LNA sugar moiety. In certain embodiments, modified oligonucleotides do not comprise more than one, two, three, four, five, six, seven, eight, or nine LNA sugar moieties. In certain embodiments, modified oligonucleotides comprise one or two wing segments that comprise a nucleoside that is not a LNA nucleoside.

In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in order from 5′ to 3′. In certain embodiments, the modified nucleotides have an internucleoside linkage motif of (from 5′ to 3′) of soooossssssssssooss, sooooossssssssssoss, sooossssssssssoooss, soosssssssssoooss, soooosssssssssoss, or sooosssssssssooss wherein each “s” represents a phosphorothioate internucleoside linkage and each “o” represents a phosphodiester internucleoside linkage.

The nucleobase sequences of SEQ ID NOs: 1090, 1166, 2484, 2485, 2487, 2493, 2496, 2497, 2498, 2533, 2534, 2535, and 2537 are complementary to an equal length portion within nucleobases 243917-244073 of SEQ ID NO: 2.

The nucleobase sequence of Compound IDs: 1250148, 1250149, 1348250, 1348251, 1348253, 1348259, 1348265, 1348266, 1348267, 1348331, 1348332, 1348333, 1348338, 1348342, 1348344, 1348345, 1348347, 1348419, 1348420, 1348421, 1348427, 1348428, 1348435, 1348436, 1348437, 1348920, 1348922, 1348923, 1348925, 1348927, 1348928, 1348929, 1348931, 1348934, 1348935, 1348937, and 1348938 are complementary to an equal length portion within nucleobases 243917-244073 of SEQ ID NO: 2.

In certain embodiments, modified oligonucleotides complementary to an equal length portion within nucleobases 243917-244073 of SEQ ID NO: 2 achieve at least 80% reduction of SCN2A RNA in vitro in the standard in vitro assay. In certain embodiments, modified oligonucleotides complementary to an equal length portion within nucleobases 243917-244073 of SEQ ID NO: 2 achieve an average of 80.5% reduction of SCN2A RNA in vitro in the standard in vitro assay. In certain embodiments, modified oligonucleotides complementary to an equal length portion within nucleobases 243917-244073 of SEQ ID NO: 2 achieve an average of 67.7% reduction of SCN2A RNA in vivo in the standard in vivo assay. In certain embodiments, modified oligonucleotides complementary to an equal length portion within nucleobases 243917-244073 of SEQ ID NO: 2 achieve an average of 62.1% reduction of SCN2A RNA in vivo in the standard in vivo assay.

### 5. Nucleobases 4389-4487 of SEQ ID NO: 1 or 247823-247921 of SEQ ID NO: 2

In certain embodiments, nucleobases 4389-4487 of SEQ ID NO: 1 or 247823-247921 of SEQ ID NO: 2 comprise a hotspot region. In certain embodiments, modified oligonucleotides are complementary to an equal length portion within nucleobases 4389-4487 of SEQ ID NO: 1 or 247823-247921 of SEQ ID NO: 2. In certain embodiments, modified oligonucleotides are 20 nucleobases in length. In certain embodiments, modified oligonucleotides are 18 nucleobases in length. In certain embodiments, modified oligonucleotides are 16, 17, 18, 19, 20, 21, or 22 nucleobases in length. In certain embodiments, modified oligonucleotides consist of 17-19 or 21-30 linked nucleosides. In certain embodiments, modified oligonucleotides are gapmers.

In certain embodiments, the gapmers are 5-10-5 MOE gapmers. In certain embodiments, the gapmers are 6-10-4 MOE gapmers. In certain embodiments, the gapmers are 4-10-6 MOE gapmers. In certain embodiments, the gapmers are 4-8-6 MOE gapmers. In certain embodiments, the gapmers are 6-8-4 MOE gapmers. In certain embodiments, the gapmers are 5-8-5 MOE gapmers. In certain embodiments, the gapmers have the sugar motif in order from 5′ to 3: eeeeeddddddddddeeeee, eeeeeeddddddddddeeee, eeeeddddddddddeeeeee, eeeeddddddddeeeeee, eeeeeeddddddddeeee, or eeeeeddddddddeeeee; wherein ‘d’ represents a 2′-β-D-deoxyribosyl sugar moiety, and ‘e’ represents a 2′-MOE sugar moiety. In certain embodiments, the gapmers comprise a 2′-substituted nucleoside in the gap. In certain embodiments, the 2′-substituted nucleoside comprises a 2′-OMe sugar moiety. In certain embodiments, the 2′-substituted nucleoside is at position 2 of the gap (5′ to 3′).

In certain embodiments, modified oligonucleotides do not comprise a bicyclic sugar moiety. In certain embodiments, modified oligonucleotides do not comprise more than one, two, three, four, five, six, seven, eight, or nine bicyclic sugar moieties. In certain embodiments, modified oligonucleotides comprise one or two wing segments that comprise a nucleoside that is not a bicyclic nucleoside. In certain embodiments, modified oligonucleotides do not comprise a LNA sugar moiety. In certain embodiments, modified oligonucleotides do not comprise more than one, two, three, four, five, six, seven, eight, or nine LNA sugar moieties. In certain embodiments, modified oligonucleotides comprise one or two wing segments that comprise a nucleoside that is not a LNA nucleoside.

In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in order from 5′ to 3′. In certain embodiments, the modified nucleotides have an internucleoside linkage motif of (from 5′ to 3′) of soooossssssssssooss, sooooossssssssssoss, sooossssssssssoooss, soosssssssssoooss, soooosssssssssoss, or sooosssssssssooss wherein each “s” represents a phosphorothioate internucleoside linkage and each “o” represents a phosphodiester internucleoside linkage.

The nucleobase sequences of SEQ ID NOs: 29, 30, 107, 108, 185, 186, 263, 264, 341, 342, 419, 420, 1796, 1871, 1948, 2025, 2101, 2178, 2254, 2330, 2503, 2517, and 2522 are complementary to an equal length portion within nucleobases 4389-4487 of SEQ ID NO: 1 or 247823-247921 of SEQ ID NO: 2.

The nucleobase sequence of Compound IDs: 910009, 910010, 910011, 910012, 910013, 910014, 910015, 910016, 910017, 910018, 910019, 910020, 1248528, 1248529, 1248530, 1248531, 1248532, 1248533, 1248534, 1248535, 1348269, 1348270, 1348271, 1348275, 1348277, 1348348, 1348353, 1348355, 1348356, 1348396, and 1348450 are complementary to an equal length portion within nucleobases 4389-4487 of SEQ ID NO: 1 or 247823-247921 of SEQ ID NO: 2.

In certain embodiments, modified oligonucleotides complementary to an equal length portion within nucleobases 4389-4487 of SEQ ID NO: 1 or 247823-247921 of SEQ ID NO: 2 achieve at least 27% reduction of SCN2A RNA in vitro in the standard in vitro assay. In certain embodiments, modified oligonucleotides complementary to an equal length portion within nucleobases 4389-4487 of SEQ ID NO: 1 or 247823-247921 of SEQ ID NO: 2 achieve an average of 71.1% reduction of SCN2A RNA in vitro in the standard in vitro assay. In certain embodiments, modified oligonucleotides complementary to an equal length portion within nucleobases 4389-4487 of SEQ ID NO: 1 or 247823-247921 of SEQ ID NO: 2 achieve an average of 63.4% reduction of SCN2A RNA in vivo in the standard in vivo assay. In certain embodiments, modified oligonucleotides complementary to an equal length portion within nucleobases 4389-4487 of SEQ ID NO: 1 or 247823-247921 of SEQ ID NO: 2 achieve an average of 59.1% reduction of SCN2A RNA in vivo in the standard in vivo assay.

### 6. Nucleobases 4774-4809 of SEQ ID NO: 1 or 254142-254177 of SEQ ID NO: 2

In certain embodiments, nucleobases 4774-4809 of SEQ ID NO: 1 or 254142-254177 of SEQ ID NO: 2 comprise a hotspot region. In certain embodiments, modified oligonucleotides are complementary to an equal length portion within nucleobases 4774-4809 of SEQ ID NO: 1 or 254142-254177 of SEQ ID NO: 2. In certain embodiments, modified oligonucleotides are 20 nucleobases in length. In certain embodiments, modified oligonucleotides are 18 nucleobases in length. In certain embodiments, modified oligonucleotides are 16, 17, 18, 19, 20, 21, or 22 nucleobases in length. In certain embodiments, modified oligonucleotides consist of 17-19 or 21-30 linked nucleosides. In certain embodiments, modified oligonucleotides are gapmers.

In certain embodiments, the gapmers are 5-10-5 MOE gapmers. In certain embodiments, the gapmers are 6-10-4 MOE gapmers. In certain embodiments, the gapmers are 4-10-6 MOE gapmers. In certain embodiments, the gapmers are 4-8-6 MOE gapmers. In certain embodiments, the gapmers are 6-8-4 MOE gapmers. In certain embodiments, the gapmers are 5-8-5 MOE gapmers. In certain embodiments, the gapmers have the sugar motif in order from 5′ to 3′: eeeeeddddddddddeeeee, eeeeeeddddddddddeeee, eeeeddddddddddeeeeee, eeeeddddddddeeeeee, eeeeeeddddddddeeee, or eeeeeddddddddeeeee; wherein ‘d’ represents a 2′-β-D-deoxyribosyl sugar moiety, and ‘e’ represents a 2′-MOE sugar moiety. In certain embodiments, the gapmers comprise a 2′-substituted nucleoside in the gap. In certain embodiments, the 2′-substituted nucleoside comprises a 2′-OMe sugar moiety. In certain embodiments, the 2′-substituted nucleoside is at position 2 of the gap (5′ to 3′).

In certain embodiments, modified oligonucleotides do not comprise a bicyclic sugar moiety. In certain embodiments, modified oligonucleotides do not comprise more than one, two, three, four, five, six, seven, eight, or nine bicyclic sugar moieties. In certain embodiments, modified oligonucleotides comprise one or two wing segments that comprise a nucleoside that is not a bicyclic nucleoside. In certain embodiments, modified oligonucleotides do not comprise a LNA sugar moiety. In certain embodiments, modified oligonucleotides do not comprise more than one, two, three, four, five, six, seven, eight, or nine LNA sugar moieties. In certain embodiments, modified oligonucleotides comprise one or two wing segments that comprise a nucleoside that is not a LNA nucleoside.

In certain embodiments, the internucleoside linkages of the modified oligonucleotides are phosphorothioate internucleoside linkages and phosphodiester internucleoside linkages.

In certain embodiments, the phosphodiester (“o”) and phosphorothioate (“s”) internucleoside linkages are arranged in order from 5′ to 3′. In certain embodiments, the modified nucleotides have an internucleoside linkage motif of (from 5′ to 3′) of soooossssssssssooss, sooooossssssssssoss, sooossssssssssoooss, soosssssssssoooss, soooosssssssssoss, or sooosssssssssooss wherein each “s” represents a phosphorothioate internucleoside linkage and each “o” represents a phosphodiester internucleoside linkage.

The nucleobase sequences of SEQ ID NOs: 1016, 1093, 1104, 1169, 1246, 1323, 1400, 1477, 1554, 1708, 1785, 1860, 1937, 2014, 1631, 2090, and 2539 are complementary to an equal length portion within nucleobases 4774-4809 of SEQ ID NO: 1 or 254142-254177 of SEQ ID NO: 2.

The nucleobase sequence of Compound IDs: 1248544, 1250225, 1250226, 1250227, 1250228, 1250229, 1250230, 1250231, 1250232, 1250233, 1250234, 1250235, 1250236, 1250237, 1250238, 1250239, 1348936, and 1348939 are complementary to an equal length portion within nucleobases 4774-4809 of SEQ ID NO: 1 or 254142-254177 of SEQ ID NO: 2.

In certain embodiments, modified oligonucleotides complementary to an equal length portion within nucleobases 4774-4809 of SEQ ID NO: 1 or 254142-254177 of SEQ ID NO: 2 achieve at least 51% reduction of SCN2A RNA in vitro in the standard in vitro assay. In certain embodiments, modified oligonucleotides complementary to an equal length portion within nucleobases 4774-4809 of SEQ ID NO: 1 or 254142-254177 of SEQ ID NO: 2 achieve an average of 89% reduction of SCN2A RNA in vitro in the standard in vitro assay. In certain embodiments, modified oligonucleotides complementary to an equal length portion within nucleobases 4774-4809 of SEQ ID NO: 1 or 254142-254177 of SEQ ID NO: 2 achieve an average of 74.8% reduction of SCN2A RNA in vivo in the standard in vivo assay. In certain embodiments, modified oligonucleotides complementary to an equal length portion within nucleobases 4774-4809 of SEQ ID NO: 1 or 254142-254177 of SEQ ID NO: 2 achieve an average of 67.8% reduction of SCN2A RNA in vivo in the standard in vivo assay.

### 7. Additional Hotspot Regions

In certain embodiments, the ranges described in the table below comprise hotspot regions. Each hotspot region begins with the nucleobase of SEQ ID NO:2 identified in the “Start Site SEQ ID NO: 2” column and ends with the nucleobase of SEQ ID NO: 2 identified in the “Stop Site SEQ ID NO: 2” column. In certain embodiments, modified oligonucleotides are complementary to an equal length portion within any of the hotspot regions 1-17, as defined in the table below. In certain embodiments, modified oligonucleotides are 20 nucleobases in length. In certain embodiments, modified oligonucleotides arm 18 nucleobases in length. In certain embodiments, modified oligonucleotides are 16, 17, 18, 19, 20, 21, or 22 nucleobases in length. In certain embodiments, modified oligonucleotides consist of 17-19 or 21-30 linked nucleosides. In certain embodiments, modified oligonucleotides are gapmers.

In certain embodiments, the gapmers are 5-10-5 MOE gapmers. In certain embodiments, the gapmers are 6-10-4 MOE gapmers. In certain embodiments, the gapmers are 4-10-6 MOE gapmers. In certain embodiments, the gapmers are 4-8-6 MOE gapmers. In certain embodiments, the gapmers are 6-8-4 MOE gapmers. In certain embodiments, the gapmers are 5-8-5 MOE gapmers. In certain embodiments, the gapmers have the sugar motif in order from 5′ to 3′: eeeeeddddddddddeeeee, eeeeeeddddddddddeeee, eeeeddddddddddeeeeee, eeeeddddddddeeeeee, eeeeeeddddddddeeee, or eeeeeddddddddeeeee; wherein ‘d’ represents a 2′-β-D-deoxyribosyl sugar moiety, and ‘e’ represents a 2′-MOE sugar moiety. In certain embodiments, the gapmers comprise a 2′-substituted nucleoside in the gap. In certain embodiments, the 2′-substituted nucleoside comprises a 2′-OMe sugar moiety. In certain embodiments, the 2′-substituted nucleoside is at position 2 of the gap (5′ to 3′).

In certain embodiments, modified oligonucleotides do not comprise a bicyclic sugar moiety. In certain embodiments, modified oligonucleotides do not comprise more than one, two, three, four, five, six, seven, eight, or nine bicyclic sugar moieties. In certain embodiments, modified oligonucleotides comprise one or two wing segments that comprise a nucleoside that is not a bicyclic nucleoside. In certain embodiments, modified oligonucleotides do not comprise a LNA sugar moiety. In certain embodiments, modified oligonucleotides do not comprise more than one, two, three, four, five, six, seven, eight, or nine LNA sugar moieties. In certain embodiments, modified oligonucleotides comprise one or two wing segments that comprise a nucleoside that is not a LNA nucleoside.

In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in order from 5′ to 3′. In certain embodiments, the modified nucleotides have an internucleoside linkage motif of (from 5′ to 3′) of soooossssssssssooss, sooooossssssssssoss, sooossssssssssoooss, soosssssssssoooss, soooosssssssssoss, or sooosssssssssooss wherein each “s” represents a phosphorothioate internucleoside linkage and each “o” represents a phosphodiester internucleoside linkage.

The nucleobase sequences of compounds listed in the “Compound IDs in range” column in the table below are complementary to SEQ ID NO: 2 within the specified hotspot region. The nucleobase sequences of the oligonucleotides listed in the “SEQ ID NOs in range” column in the table below are complementary to the target sequence, SEQ ID NO: 2, within the specified hotspot region.

In certain embodiments, modified oligonucleotides complementary to nucleobases within the hotspot region achieve at least “Min.% Red, in vitro” (minimum % reduction, relative to untreated control cells) of SCN2A RNA in vitro in the standard in vitro assay, as indicated in the table below. In certain embodiments, modified oligonucleotides complementary to nucleobases within the hotspot region achieve an average of “Avg.% Red, in vitro” (average % reduction, relative to untreated control cells) of SCN2A RNA in vitro in the standard in vitro assay, as indicated in the table below. In certain embodiments, modified oligonucleotides complementary to nucleobases within the hotspot region achieve a maximum of “Max. % Red, in vitro” (maximum % reduction, relative to untreated control cells) of SCN2A RNA in vitro in the standard in vitro assay, as indicated in the table below. In certain embodiments, modified oligonucleotides complementary to nucleobases within the hotspot region achieve an average of “Avg. % Red, in vivo Cortex” (average % reduction, relative to PBS-treated animals) of SCN2A RNA in vivo in the standard in vivo assay in cortical tissue, as indicated in the table below. In certain embodiments, modified oligonucleotides complementary to nucleobases within the hotspot region achieve an average of “Avg. % Red, in vivo Spinal” (average % reduction, relative to PBS-treated animals) of SCN2A RNA in vivo in the standard in vivo assay in spinal cord tissue, as indicated in the table below. “n.d.” indicates that no in vivo data is available for compounds within that range. In other cases, average reduction in vivo includes a subset of the compounds in any given hotspot, as not all compounds were tested in vivo.

TABLE 1
SCN2A Hotspots

| | | | | | | Avg % | Avg % | | |
|---|---|---|---|---|---|---|---|---|---|
| | Start Site | Stop Site | Min % | Max % | Avg % | Red. | Red. | | |
| Hotspot | SEQ ID | SEQ ID | Red. in | Red. in | Red. in | in vivo | in vivo | Compound IDs | SEQ ID NOs |
| ID | NO: 2 | NO: 2 | vitro | vitro | vitro | Cortex | Spinal | in range | in range |
| 1 | 199863 | 199905 | 53 | 83 | 69.9 | 77.1 | 63.2 | 909979, 1248427, | 336, 488, 2021, |
| | | | | | | | | 1248428, 1248429, | 2097, 2174, 2250, |
| | | | | | | | | 1248430, 1248431, | 2326, 2403, 2499, |
| | | | | | | | | 1248432, 1248433, | 2500, 2501, 2502, |
| | | | | | | | | 1348279, 1348282, | and 2526 |
| | | | | | | | | 1348286, 1348297, | |
| | | | | | | | | 1348328, 1348343, | |
| | | | | | | | | 1348358, 1348360, | |
| | | | | | | | | 1348361, 1348362, | |
| | | | | | | | | 1348364, 1348365, | |
| | | | | | | | | 1348366, 1348367, | |
| | | | | | | | | 1348378, and | |
| | | | | | | | | 1348380 | |
| 2 | 227493 | 227551 | 75 | 88 | 81.6 | 76.6 | 67.2 | 909989, 909990, | 181, 259, 643, 720, |
| | | | | | | | | 1248487, 1248488, | 796, 2504, 2505, |
| | | | | | | | | 1248489, 1348289, | 2506, 2507, 2508, |
| | | | | | | | | 1348290, 1348291, | 2509, 2510, 2511, |
| | | | | | | | | 1348292, 1348295, | 2512, 2513, 2514, |
| | | | | | | | | 1348298, 1348302, | and 2521 |
| | | | | | | | | 1348303, 1348304, | |
| | | | | | | | | 1348306, 1348307, | |
| | | | | | | | | 1348369, 1348370, | |
| | | | | | | | | 1348371, 1348373, | |
| | | | | | | | | 1348374, 1348375, | |
| | | | | | | | | 1348376, 1348377, | |
| | | | | | | | | 1348381, 1348382, | |
| | | | | | | | | 1348383, 1348384, | |
| | | | | | | | | 1348385, 1348386, | |
| | | | | | | | | 1348387, 1348405, | |
| | | | | | | | | 1348411, 1348423, | |
| | | | | | | | | 1348439, 1348440, | |
| | | | | | | | | 1348441, 1348442, | |
| | | | | | | | | 1348443, 1348444, | |
| | | | | | | | | 1348446, 1348447, | |
| | | | | | | | | and 1348456 | |
| 3 | 243124 | 243204 | 51 | 90 | 71.4 | 61.3 | 61.5 | 1248507, 1248508, | 491, 567, 644, 721, |
| | | | | | | | | 1248509, 1248510, | 797, 2177, 2253, |
| | | | | | | | | 1248511, 1248512, | 2315, 2329, 2406, |
| | | | | | | | | 1248513, 1248514, | and 2527 |
| | | | | | | | | 1248515, 1250138, | |
| | | | | | | | | 1348299, 1348379, | |
| | | | | | | | | 1348388, and | |
| | | | | | | | | 1348397 | |
| 4 | 243917 | 244073 | 80 | 81 | 80.5 | 67.7 | 62.1 | 1250148, 1250149, | 1090, 1166, 2484, |
| | | | | | | | | 1348250, 1348251, | 2485, 2487, 2493, |
| | | | | | | | | 1348253, 1348259, | 2496, 2497, 2498, |
| | | | | | | | | 1348265, 1348266, | 2533, 2534, 2535, |
| | | | | | | | | 1348267, 1348331, | and 2537 |
| | | | | | | | | 1348332, 1348333, | |
| | | | | | | | | 1348338, 1348342, | |
| | | | | | | | | 1348344, 1348345, | |
| | | | | | | | | 1348347, 1348419, | |
| | | | | | | | | 1348420, 1348421, | |
| | | | | | | | | 1348427, 1348428, | |
| | | | | | | | | 1348435, 1348436, | |
| | | | | | | | | 1348437, 1348920, | |
| | | | | | | | | 1348922, 1348923, | |
| | | | | | | | | 1348925, 1348927, | |
| | | | | | | | | 1348928, 1348929, | |
| | | | | | | | | 1348931, 1348934, | |
| | | | | | | | | 1348935, 1348937, | |
| | | | | | | | | and 1348938 | |
| 5 | 247823 | 247921 | 27 | 92 | 71.1 | 63.4 | 59.1 | 910009, 910010, | 29, 30, 107, 108, |
| | | | | | | | | 910011, 910012, | 185, 186, 263, 264, |
| | | | | | | | | 910013, 910014, | 341, 342, 419, 420, |
| | | | | | | | | 910015, 910016, | 1796, 1871, 1948, |
| | | | | | | | | 910017, 910018, | 2025, 2101, 2178, |
| | | | | | | | | 910019, 910020, | 2254, 2330, 2503, |
| | | | | | | | | 1248528, 1248529, | 2517, and 2522 |
| | | | | | | | | 1248530, 1248531, | |
| | | | | | | | | 1248532, 1248533, | |
| | | | | | | | | 1248534, 1248535, | |
| | | | | | | | | 1348269, 1348270, | |
| | | | | | | | | 1348271, 1348275, | |
| | | | | | | | | 1348277, 1348348, | |
| | | | | | | | | 1348353, 1348355, | |
| | | | | | | | | 1348356, 1348396, | |
| | | | | | | | | and 1348450 | |
| 6 | 254142 | 254177 | 51 | 89 | 71.7 | 74.8 | 67.8 | 1248544, 1250225, | 1016, 1093, 1104, |
| | | | | | | | | 1250226, 1250227, | 1169, 1246, 1323, |
| | | | | | | | | 1250228, 1250229, | 1400, 1477, 1554, |
| | | | | | | | | 1250230, 1250231, | 1708, 1785, 1860, |
| | | | | | | | | 1250232, 1250233, | 1937, 2014, 1631, |
| | | | | | | | | 1250234, 1250235, | 2090, and 2539 |
| | | | | | | | | 1250236, 1250237, | |
| | | | | | | | | 1250238, 1250239, | |
| | | | | | | | | 1348936, and | |
| | | | | | | | | 1348939 | |
| 7 | 168911 | 168945 | 71 | 93 | 81.2 | n.d. | n.d. | 909945, 909946, | 18, 96, 485, 561, |
| | | | | | | | | 1248352, 1248353, | 638, 715, 791, 868, |
| | | | | | | | | 1248354, 1248355, | 2247, 2323, and 2400 |
| | | | | | | | | 1248356, 1248357, | |
| | | | | | | | | 1248358, 1248359, | |
| | | | | | | | | and 1248360. | |
| 8 | 170026 | 170061 | 65 | 84 | 82.3 | n.d. | n.d. | 909947, 1248366, | 174, 1328, 1405, |
| | | | | | | | | 1248367, 1248368, | 1482, 1559, 1636, |
| | | | | | | | | 1248369, 1248370, | 1713, 1790, 1865, |
| | | | | | | | | 1248371, 1248372, | 1942, and 2019 |
| | | | | | | | | 1248373, 1248374, | |
| | | | | | | | | and 1248375 | |
| 9 | 170174 | 170200 | 69 | 93 | 81.6 | n.d. | n.d. | 910246, 1249167, | 302, 1513, 1667, |
| | | | | | | | | 1249168, 1249169, | 1744, 1819, 1896, |
| | | | | | | | | 1249170, 1249171, | and 1973 |
| | | | | | | | | and 1249172 | |
| 10 | 176724 | 176751 | 75 | 94 | 86.7 | n.d. | n.d. | 910256, 910257, | 148, 226, 1364, |
| | | | | | | | | 1249294, 1249295, | 1441, 1518, 1595, |
| | | | | | | | | 1249296, 1249297, | 1672, and 1749 |
| | | | | | | | | 1249298, and | |
| | | | | | | | | 1249299 | |
| 11 | 180772 | 180801 | 67 | 93 | 78.6 | n.d. | n.d. | 910263, 1249423, | 227, 1292, 1369, |
| | | | | | | | | 1249424, 1249425, | 1446, 1523, 1600, |
| | | | | | | | | 1249426, 1249427, | 1677, 1754, and |
| | | | | | | | | 1249428, 1249429, | 1829 |
| | | | | | | | | and 1249430 | |
| 12 | 183519 | 183562 | 73 | 97 | 83.1 | n.d. | n.d. | 909954, 909955, | 20, 98, 253, 332, |
| | | | | | | | | 909956, 909957, | 410, 1406, 1483, |
| | | | | | | | | 909958, 1248393, | 1560, 1637, 1714, |
| | | | | | | | | 1248394, 1248395, | 1791, 1866, and |
| | | | | | | | | 1248396, 1248397, | 1943 |
| | | | | | | | | 1248398, 1248399, | |
| | | | | | | | | and 1248400 | |
| 13 | 183968 | 184016 | 60 | 95 | 77.1 | n.d. | n.d. | 910269, 1249480, | 228, 1679, 1756, |
| | | | | | | | | 1249481, 1249482, | 1831, 1908, 1985, |
| | | | | | | | | 1249483, 1249484, | 2061, 2138, 2214, |
| | | | | | | | | 1249485, 1249486, | and 2290 |
| | | | | | | | | 1249487, and | |
| | | | | | | | | 1249488 | |
| 14 | 188630 | 188668 | 68 | 86 | 77.1 | n.d. | n.d. | 909962, 909963, | 21, 411, 1407, |
| | | | | | | | | 1248419, 1248420, | 1484, 1561, 1638, |
| | | | | | | | | 1248421, 1248422, | and 1715 |
| | | | | | | | | and 1248423 | |
| 15 | 199912 | 199962 | 72 | 94 | 82.7 | n.d. | n.d. | 909980, 909981, | 24, 414, 871, 948, |
| | | | | | | | | 1248438, 1248439, | 1025, and 1100 |
| | | | | | | | | 1248440, and | |
| | | | | | | | | 1248441 | |
| 16 | 202877 | 202906 | 70 | 92 | 81.5 | n.d. | n.d. | 1249708, 1249709, | 1226, 1303, 1380, |
| | | | | | | | | 1249710, 1249711, | 1457, 1534, and |
| | | | | | | | | 1249712, and | 1611 |
| | | | | | | | | 1249713 | |
| 17 | 227419 | 227450 | 67 | 92 | 80.6 | n.d. | n.d. | 909985, 909986, | 25, 337, 415, 490, |
| | | | | | | | | 909987, 1248480, | 566, 2099, 2176, |
| | | | | | | | | 1248481, 1248482, | 2252, 2328, and |
| | | | | | | | | 1248483, 1248484, | 2405 |
| | | | | | | | | 1248485, and | |
| | | | | | | | | 1248486 | |

### IX. Certain Comparator Compounds

Comparator Compound No. 1506060 was selected as a comparator compound. Comparator Compound No. 1506060, previously described in WO2020/041348, incorporated herein by reference, is a 4-8-4 LNA gapmer with the sequence (from 5′ to 3′) TGGGTCTCTTAGCTTT (SEQ ID NO: 2540), wherein the central gap segment consists of eight 2′-β-D-deoxynucleosides, the 5′ and 3′ wing segments each consist of four LNA modified nucleosides, and each internucleoside linkage is a phosphorothioate internucleoside linkage.

In certain embodiments, compounds described herein are more tolerable relative to Comparator Compound No. 1506060.

For example, as described in Example 4 of WO/2022/032060, which is herein incorporated by reference in its entirety, Comparator Compound No. 1506060 had a 3-hour FOB of 6.00 in mice, whereas Compound Nos. 1348290, 1348331, and 1348347 each had a 3-hour FOB of 0.00 in mice, and Compound Nos. 1348259, 1348289, and 1348937 each had a 3-hour FOB of 0 or 1.00 in mice. Therefore, certain compounds described herein are more tolerable than Comparator Compound No. 1506060 in this assay.

### Non-Limiting Disclosure and Incorporation by Reference

Each of the literature and patent publications listed herein is incorporated by reference in its entirety. While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, all cis- and trans-isomers and tautomeric forms of the compounds herein are also included unless otherwise indicated. Oligomeric compounds described herein include chirally pure or enriched mixtures as well as racemic mixtures. For example, oligomeric compounds having a plurality of phosphorothioate internucleoside linkages include such compounds in which chirality of the phosphorothioate internucleoside linkages is controlled or is random. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### Methods of Treatment

Disclosed herein are methods of treating SCN2A-related disorders, such as early-onset developmental or epileptic encephalopathy (DEE).

In one aspect, the SCN2A-related disorder is early-onset developmental or epileptic encephalopathy (DEE) and the method comprises administering a therapeutically effective amount of a oligomeric compound, as described herein, to a subject in need thereof, wherein the oligomeric compound is administered intrathecally to the subject at a dosage of about 1 mg to about 100 mg.

The methods disclosed herein may be used to ameliorate one or more symptoms of early-onset DEE, including, for example, seizures, limitations in communication, such as delayed language and speech; autonomic dysfunction; developmental delay; gastrointestinal abnormalities; movement disorders, such as choreoathetosis, dystonia, ataxia; anxiety; sensory issues; urinary retention problems; irritability; sleep problems (e.g., inability to fall asleep and inability to stay asleep); and behavior issues. Seizures include focal, clonic, tonic, and generalized tonic and clonic seizures, prolonged seizures (often lasting longer than 10 minutes), and frequent seizures.

In certain aspects, the method comprises a dose escalation phase (or a titration phase) and a maintenance phase. Typically, the dose escalation phase is used to determine a maximum dose for the subject and the maintenance phase is used to administer the maximum dose to the subject on an ongoing basis. In certain aspects, the method does not include a dose escalation phase and a selected dose (also referred to herein as a maintenance dose) is administered to the subject on an ongoing basis.

In certain embodiments, the dosage of the oligomeric compound is about 1 mg to about 50 mg. In certain embodiments, the dosage of the oligomeric compound is about 1 mg to about 25 mg. In certain embodiments, the dosage of the oligomeric compound is about 1 mg to about 15 mg. In certain embodiments, the dosage of the oligomeric compound is about 1 mg to about 10 mg. In certain embodiments, the dosage of the oligomeric compound is about 5 mg to about 50 mg. In certain embodiments, the dosage of the oligomeric compound is about 5 mg to about 40 mg. In certain embodiments, the dosage of the oligomeric compound is about 5 mg to about 30 mg. In certain embodiments, the dosage of the oligomeric compound is about 5 mg to about 25 mg. In certain embodiments, the dosage of the oligomeric compound is about 5 mg to about 20 mg. In certain embodiments, the dosage of the oligomeric compound is about 5 mg to about 15 mg. In certain embodiments, the dosage of the oligomeric compound is about 5 mg to about 10 mg. In certain embodiments, the dosage of the oligomeric compound is about 10 mg to about 50 mg. In certain embodiments, the dosage of the oligomeric compound is about 10 mg to about 25 mg. In certain embodiments, the dosage of the oligomeric compound is about 15 mg to about 50 mg. In certain embodiments, the dosage of the oligomeric compound is about 15 mg to about 35 mg. In certain embodiments, the dosage of the oligomeric compound is about 15 mg to about 25 mg.

In certain embodiments, the dosage of the oligomeric compound is at least 0.25 mg. In certain embodiments, the dosage of the oligomeric compound is at least 0.5 mg. In certain embodiments, the dosage of the oligomeric compound is at least 0.75 mg. In certain embodiments, the dosage of the oligomeric compound is at least 1 mg. In various embodiments, the dosage of the oligomeric compound administered to a subject is at least 0.25 mg, at least 0.5 mg, at least 0.75 mg, at least 1 mg, at least 1.5 mg, at least 2 mg, at least 2.5 mg, at least 3 mg, at least 4 mg, at least 5 mg, at least 6 mg, at least 7 mg, at least 8 mg, at least 9 mg, at least 10 mg, at least 11 mg, at least 12 mg, at least 13 mg, at least 14 mg, at least 15 mg, at least 16 mg, at least 17 mg, at least 18 mg, at least 19 mg, or at least 20 mg.

In certain embodiments, the dosage of the oligomeric compound is at least 0.251 mg and no more than 50, 45, 40, 35, 30, 25, or 20, 15, 10, 5, 2.5, or 1 mg. In various embodiments, the dosage of the oligomeric compound is at least 0.25 mg, at least 0.5 mg, at least 0.75 mg, at least 1 mg, at least 1.5 mg, at least 2 mg, at least 2.5 mg, at least 3 mg, at least 4 mg, at least 5 mg, at least 6 mg, at least 7 mg, at least 8 mg, at least 9 mg, at least 10 mg, at least 11 mg, at least 12 mg, at least 13 mg, at least 14 mg, at least 15 mg, at least 16 mg, at least 17 mg, at least 18 mg, or at least 19 mg, and no more than 50, 45, 40, 35, 30, 25, or 20 mg. In certain embodiments, the dosage of the oligomeric compound is at least 20 mg and no more than 50, 45, 40, 35, 30, or 25 mg. In certain embodiments, the dosage of the oligomeric compound is at least 0.25 mg and no more than 2.0 mg or no more than 1 mg.

In certain embodiments, the dosage of the oligomeric compound is at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg.

In various embodiments, the dosage of the oligomeric compound is at least 5 mg, at least 10 mg, at least 15 mg, or at least 20 mg and no more than 50, 45, 40, 35, 30 mg, or 25 mg. In various embodiments, the dosage of the oligomeric compound is at least 25 mg or 30 mg and no more than 60, 55, 50, 45, 40, or 35 mg. In various embodiments, the dosage of the oligomeric compound is at least 35 mg or at least 40 mg and no more than 70, 65, 60, 55, 50, or 45 mg. In various embodiments, the dosage of the oligomeric compound is at least 45 mg or at least 50 mg and no more than 80, 75, 70, 65, 60, or 55 mg. In various embodiments, the dosage of the oligomeric compound is at least 55 mg or at least 60 mg and no more than 90, 85, 80, 75, 70, or 65 mg. In various embodiments, the dosage of the oligomeric compound is at least 65 mg or at least 70 mg and no more than 100, 95, 90, 85, 80, or 75 mg. In various embodiments, the dosage of the oligomeric compound is at least 75 mg or at least 80 mg and no more than 110, 105, 100, 95, 90, or 85 mg. In various embodiments, the dosage of the oligomeric compound is at least 85 mg or at least 90 mg and no more than 120, 115, 110, 105, 100, or 95 mg. In various embodiments, the dosage of the oligomeric compound is at least 95 mg or at least 100 mg and no more than 130, 125, 120, 115, 110, or 100 mg.

In certain embodiments, the dosage of the oligomeric compound is at least 0.1 mg/kg. In various embodiments, the dosage of the oligomeric compound administered to a subject is at least 0.2 mg/kg, at least 0.3 mg/kg, at least 0.4 mg/kg, at least 0.5 mg/kg, at least 0.6 mg/kg, at least 0.7 mg/kg, at least 0.8 mg/kg, at least 0.9 mg/kg, at least 1.0 mg/kg, at least 1.1 mg/kg, at least 1.2 mg/kg, at least 1.3 mg/kg, at least 1.4 mg/kg, at least 1.5 mg/kg, at least 1.6 mg/kg, at least 1.7 mg/kg, at least 1.8 mg/kg, at least 1.9 mg/kg, or at least 2.0 mg/kg. In certain embodiments, the dosage of the oligomeric compound is at least 0.1 mg/kg and no more than 10 mg/kg, no more than 5 mg/kg, no more than 2.0 mg/kg, or no more than 1.0 mg/kg.

In various embodiments, the dosage of the oligomeric compound administered to a subject is at least 5 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg.

In various embodiments, the dosage of the oligomeric compound is at least 5 mg/kg or at least 10 mg/kg and no more than 20 mg/kg or 15 mg/kg. In certain embodiments, the dosage of the oligomeric compound is at least 15 mg/kg or at least 20 mg/kg and no more than 30 mg/kg or 25 mg/kg. In certain embodiments, the dosage of the oligomeric compound is at least 25 mg/kg or at least 30 mg/kg and no more than 40 mg/kg or 35 mg/kg. In certain embodiments, the dosage of the oligomeric compound is at least 35 mg/kg or at least 40 mg/kg and no more than 50 mg/kg or 45 mg/kg. In certain embodiments, the dosage of the oligomeric compound is at least 45 mg/kg or at least 50 mg/kg and no more than 60 mg/kg or 55 mg/kg. In certain embodiments, the dosage of the oligomeric compound is at least 55 mg/kg or at least 60 mg/kg and no more than 70 mg/kg or 65 mg/kg. In certain embodiments, the dosage of the oligomeric compound is at least 65 mg/kg or at least 70 mg/kg and no more than 80 mg/kg or 75 mg/kg. In certain embodiments, the dosage of the oligomeric compound is at least 75 mg/kg or at least 80 mg/kg and and no more than 90 mg/kg or 85 mg/kg. In certain embodiments, the dosage of the oligomeric compound is at least 85 mg/kg or at least 90 mg/kg and no more than 100 mg/kg or 95 mg/kg. In certain embodiments, the dosage of the oligomeric compound is at least 95 mg/kg or at least 100 mg/kg and no more than 150 mg/kg or 125 mg/kg.

In one aspect, the dosage of the oligomeric compound is titrated one or more times. In certain embodiments, the dosage is increased 1.25-fold to 5-fold between each titrated dose. In certain embodiments, the dosage is increased 1.25-fold to 2-fold, 1.5-fold to 3-fold, 1.5-fold to 2.5-fold, or 1.5-fold to 2-fold between each titrated dose. In certain embodiments, the dosage is increased 1.25-fold to 1.75-fold between each titrated dose. In certain embodiments, the dosage is increased 2-fold, up to 8 mg, followed by no more than a 1.5-fold increase for all subsequent doses.

In certain embodiments, during the dose escalation phase, the dosage of the oligomeric compound is titrated from an initial dose of about 1 mg to a maximum dose of at least about 2 mg, about 4 mg, about 8 mg, about 10 mg, about 12 mg, about 15 mg, about 18 mg, about 20 mg, about 15 mg, about 25 mg, about 30 mg, about 32 mg, about 35 mg, about 40 mg, about 50 about mg, about 55 mg, about 60 mg, about 65 mg, about 70 mg, about 75 mg, about 80 mg, about 85 mg, about 90 mg, about 100 mg, or more. In certain embodiments, during the dose escalation phase, the dosage of the oligomeric compound is titrated from an initial dose of about 1 mg, about 2 mg, about 3 mg, about 4 mg, about 5 mg, about 6 mg, about 7 mg, about 8 mg, about 9 mg, about, 10 mg, about 11 mg, about 12 mg, about 13 mg, about 14 mg, or about 15 mg to a maximum dose of at least about 20 mg, about 25 mg, about 30 mg, about 35 mg, about 40 mg, about 50 mg, about 60 mg, about 65 mg, about 70 mg, about 75 mg, about 80 mg, about 85 mg, about 90 mg, about 95 mg, about 100 mg, or more. In certain embodiments, the maximum dose is about 4-100 mg, about 4-50 mg, about 4-25 mg, about 4-15 mg, about 8-100 mg, about 8-75 mg, about 8-50 mg, about 8-25 mg, or about 8-15 mg. In certain embodiments, the maximum dose is about 15-100 mg, about 15-75 mg, about 15-50 mg, about 15-40 mg, about 15-30 mg, about 15-25 mg. In certain embodiments, during the dose escalation phase, the dosage of the oligomeric compound is titrated from an initial dose of about 1 mg to a maximum dose of at least about 15 mg. In certain embodiments, during the dose escalation phase, the dosage of the oligomeric compound is titrated from an initial dose of about 1 mg to a maximum dose of about 32 mg. In certain embodiments, during the dose escalation phase, the dosage of the oligomeric compound is titrated from an initial dose of about 1 mg to a maximum dose of about 32-64 mg. In certain embodiments, the dosage of the oligomeric compound is titrated from an initial dose of about 1 mg to a maximum dose of at least 64 mg.

In certain embodiments, during the dose escalation phase, the dosage of the oligomeric compound is titrated from an initial dose of about 1 mg to a cumulative dose of at least about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, or more. In certain embodiments, during the dose escalation phase, the dosage of the oligomeric compound is titrated from an initial dose of about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 25 mg, or about 30 mg to a cumulative dose of at least about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, or more. In certain embodiments, during the dose escalation phase, the cumulative dose is about 40-200 mg, about 50-200 mg, about 50-175 mg, about 50-150 mg, about 50-125 mg, about 50-100 mg, about 50-75 mg, about 40-50 mg, about 75-200 mg, about 75-175 mg, about 75-150 mg, about 75-125 mg, about 75-100 mg, about 100-200 mg, about 100-175 mg, about 100-150 mg, about 100-125 mg, about 125-200 mg, about 125-175 mg, about 125-150 mg, about 150-200 mg, or about 150-175 mg. In certain embodiments, during the dose escalation phase, the dosage of the oligomeric compound is titrated from an initial dose of about 1 mg to a cumulative dose of at least about 40 mg or 50 mg. In certain embodiments, during the dose escalation phase, the dosage of the oligomeric compound is titrated from an initial dose of about 1 mg to a cumulative dose of at least about 100 mg. In certain embodiments, during the dose escalation phase, the dosage of the oligomeric compound is titrated from an initial dose of about 1 mg to a cumulative dose of at least about 40-100 mg or 50-100 mg.

In one aspect, either the maximum dose or cumulative dose of the dose escalation phase is reached after administration of multiple titrated doses of the oligomeric compound. In certain embodiments, the cumulative dose of the dose escalation phase is reached after administration of 2-20 titrated doses of the oligomeric compound, including after 2, 3, 4, 5, 6, 7, 8, 9, 10, 11, 12, 13, 14, 15, 16, 17, 18, 19, or 20 titrated doses of the oligomeric compound. In certain embodiments, either the maximum dose or cumulative dose of the dose escalation phase is reached after administration of 4-16, 4-12, 4-10, 4-8, 5-15, 5-12, 5-10, 5-8, 6-12, 6-10, 8-12, or 8-10 titrated doses of the oligomeric compound. In certain embodiments, either the maximum dose or cumulative dose of the dose escalation phase is reached after administration of up to about 4 to about 12 titrated doses of the oligomeric compound.

In one aspect, each administration of the titrated dose of the oligomeric compound is separated by about 3-20 weeks. In certain embodiments, each administration of the titrated dose of the oligomeric compound is separated by about 1 week. 2, 3, 4, 5, 6, 7, 8, 9, 10, 11, 12, 13, 14, 15, 16, 17, 18, 19, or 20 weeks. In certain embodiments of the dose escalation phase, the titrated dose of the oligomeric compound is administered about every 4-6 weeks. In certain embodiments, each administration of the titrated dose of the oligomeric compound is separated by at least about 6 weeks. In certain embodiments, each administration of the titrated dose of the oligomeric compound is separated by at least about 4 weeks. In other embodiments, each administration of the titrated dose of the oligomeric compound is separated by at least about 2 weeks. In certain embodiments, a titrated dose of the oligomeric compound is administered no more frequently than every 6 weeks. In other embodiments, a titrated dose of the oligomeric compound is administered no more frequently than every 4 weeks. In other embodiments, a titrated dose of the oligomeric compound is administered no more frequently than every 2 weeks.

In some embodiments, the oligomeric compound is administered to a subject every week. In some embodiments, the oligomeric compound is administered to a subject every 2 weeks. In some embodiments, the oligomeric compound is administered to a subject every 3 weeks. In some embodiments, the oligomeric compound is administered to a subject every 4 weeks. In some embodiments, the oligomeric compound is administered to a subject every 6 weeks. In some embodiments, the oligomeric compound is administered to a subject every 8 weeks. In some embodiments, the oligomeric compound is administered to a subject every 10 weeks. In some embodiments, the oligomeric compound is administered to a subject every 12 weeks.

In certain embodiments, during the dose escalation phase, the dosage of the oligomeric compound is titrated from an initial dose of about 1 mg to a maximum dose of about 15 mg. In certain embodiments, during the dose escalation phase, the dosage of the oligomeric compound is titrated from a first dose of about 1 mg to a second dose of about 2 mg, and optionally to a third dose of about 4 mg, and optionally to a fourth dose of up to about 8 mg, and optionally to a fifth dose of up to about 12 mg, and optionally to a sixth dose of up to about 15 mg. In certain embodiments, the fourth dose of about 8 mg is titrated to a fifth dose of about 15 mg. In certain embodiments, 2-6 weeks separate the administration of each titrated dose. In other embodiments, the titration can continue up to a maximum dose of 100 mg.

In one aspect, the method of treatment further comprises administering a maintenance dose during a maintenance phase. In certain embodiments, the maintenance phase follows the dose escalation phase. In certain embodiments, the maintenance dose is the maximum dose following the dose escalation phase. In other embodiments, the maintenance dose is selected and administered to a subject without the subject undergoing a dose escalation phase.

In certain embodiments, the maintenance dose is administered up to about every 16-20 weeks or every 2-20 weeks. In certain embodiments, the maintenance dose is administered up to about every 1, 2, 3, 4, 5, 6, 7, 8, 9, 10, 11, 12, 13, 14, 15, 16, 17, 18, 19, or 20 weeks. In certain embodiments, the maintenance dose is administered up to about every 1-16 weeks, 2-16 weeks, 2-8 weeks, 6-18 weeks, 6-16 weeks, 6-14 weeks, 6-12 weeks, 6-10 weeks, 8-18 weeks, 8-16 weeks, 8-14 weeks, 8-12 weeks, 8-10 weeks, 10-18 weeks, 10-16 weeks, 10-14 weeks, or 10-12 weeks. In certain embodiments, the maintenance dose is administered up to about every 12 weeks.

In one aspect of the methods of treatment, the maintenance dose is administered after the maximum dose, as described herein, has been reached during the dose escalation phase. In certain embodiments, the maintenance dose is administered after the maximum dose of about 8-15 mg has been reached. In certain embodiments, the maintenance dose is administered after the maximum dose of about 15 mg has been reached. In certain embodiments, the maintenance dose is administered after a maximum dose of greater than 15 mg has been reached. In certain embodiments, the maintenance dose is administered after a maximum dose of about 15-32 mg has been reached. In certain embodiments, the maintenance does is administered after a maximum dose of about 30-50 mg has been reached. In certain embodiments, the maintenance dose is administered after a maximum dose of greater than 50 mg has been reached.

In one aspect, multiple maintenance doses are administered. In certain embodiments each administration of the maintenance dose is separated by about 8-20 weeks. In certain embodiments, each administration of the maintenance dose is separated by about 8, 9, 10, 11, 12, 13, 14, 15, 16, 17, 18, 19, or 20 weeks. In certain embodiments, each administration of the maintenance dose is separated by about 8-16, 8-14, 8-12, 8-10, 10-16, 10-14, or 10-12 weeks. In certain embodiments, the maintenance dose is administered at least about every 12 weeks.

In one aspect, maintenance doses are administered over a period of 6 months or greater. In certain embodiments, maintenance doses are administered for the remainder of a subject's life. In certain embodiments, maintenance doses are administered for at least 1-50 years. In certain embodiments, maintenance doses are administered for at least 1, 2, 3, 4, 5, 6, 7, 8, 9, or 10 years. In certain embodiments, maintenance doses are administered for at least 2-50 years, 2-40 years, 2-35 years, 2-30 years, 2-25 years, 2-20 years, 2-15 years, 2-10 years, or 2-5 years. In certain embodiments, maintenance doses are administered for at least 2 years.

In one aspect, the method of treatment comprises inhibiting the expression of SCN2A in neuronal cells in the subject. In various aspects, the method of treatment comprises inhibiting the expression of SCN2A mRNA in the subject by about 10% to about 90%, about 35% to about 80%, about 20%, about 25%, about 30%, about 35%, about 40%, about 45%, about 50%, about 55%, about 60%, about 65%, about 70%, about 75%, about 80%, about 85%, about 90%, about 95%, or more than about 95% but less than 100%. In other aspects, about 25% to about 50% or about 35% to about 40% knockdown of SCN2A mRNA is achieved.

In one aspect, the subject has been diagnosed with early-onset DEE prior to three months of age. In certain embodiments, the subject has been diagnosed with early-onset DEE prior to two months or prior to one month of age. In certain embodiments, the subject has been diagnosed with early-onset DEE in utero. Typically, the diagnosis comprises determining that the subject carries a SCN2A mutation prior to administering the oligomeric compound. Any SCN2A mutation can be used to diagnose early-onset DEE, including any known SCN2A mutation and any SCN2A mutation identified in the future. In certain embodiments, the SCN2A mutation comprises one or more of A263V, E430A, E430G, R1882Q, G879R, G1593R, K1502N, V1601L, G211D, S1780I, D343H, R1626Q, G882E, M1545V, L210Q, Q1479H, N1662D, F1597L, V423L, A215T, I891T, or a combination thereof. In some embodiments, the SCN2A mutation comprises one or more of Q1531K, L1563V, E1321K, Y1589C, M252V, R223E, L1330F, V208E, R36G, R1882G, D343G, V261L, F1651C, R1319Q, A263V, Q383E, V1325I, K908E, V261M, S987I, R1629H, R1882Q, M1338T, E999K, R856Q, V423L, S1336Y, R1626Q, G882E, N212D, E1211K, D195G, L1342P, R220Q, R853Q, R1435*, K503fs*, R937C, or a combination thereof.

In one aspect, the subject is a human or non-human animal. In certain embodiments, the subject is a human. In certain embodiments, the subject is a human from 2 years old to 18 years old. In other embodiments, the subject is a human older than 2 years old or younger than 18 years old. In other embodiments, the subject is a human older than 18 years old.

In certain embodiments, the subject is a human younger than 2 years old. In certain embodiments, the subject is a human infant. In further embodiments, the infant is 6 months or younger, 5 months or younger, 4 months or younger, 3 months or younger, 2 months or younger or 1 month or younger.

In certain embodiments, the subject is a human newborn. In further embodiments, the newborn is a full term newborn, i.e., born after 39 or more weeks of gestation. In other embodiments, the newborn is a premature newborn, i.e., born after less than 39 weeks of gestation. In some embodiments, the subject is a human premature newborn born after less than 38 weeks, less than 37 weeks, less than 36 weeks, less than 35 weeks, less than 34 weeks, less than 33 weeks, less than 32 weeks, or less than 31 weeks, less than 30 weeks, or less than 28 weeks of gestation.

An oligomeric compound described herein may be administered to a subject in the context of the present invention in combination with another agent or therapy, e.g., an antiepilepsy agent. Non-limiting examples of the anti-epilepsy agents include brivaracetam, carbamazepine, clobazam, clonazepam, diazepam, divalproex, eslicarbazepine, ethosuximide, ezogabine, felbamate, gabapentin, lacosamide, lamotrigine, levetiracetam, lorazepam, oxcarbezepine, permpanel, phenobarbital, phenytoin, pregabalin, primidone, rufinamide, tigabine, topiramate, valproic acid, vigabatrin, zonisamide, and cannabidiol. In one specific embodiment, an oligomeric compound is administered in combination with carbamazepine.

In addition to intrathecal administration, the oligomeric compounds described herein may also be administered, for example, by oral, parenteral, intracerebroventricular, intraparenchymal, buccal, sublingual, nasal, rectal, patch, pump, or transdermal administration. Parenteral administration includes intravenous, intraperitoneal, subcutaneous, intramuscular, transepithelial, nasal, intrapulmonary, intrathecal, intracisternal, intracerebroventricular, intraparenchymal, rectal, and topical modes.

Other SCN2A disorders can be treated according to the methods disclosed in this application include developmental or epileptic encephalopathy (DEE), such as, Ohtahara Syndrome; epilepsy with migrating focal seizures of infancy (EIMFS); infantile and childhood DEE, for example West Syndrome and Lennon-Gastaut Syndrome; Dravet Syndrome; Idiopathic/Generic Generalized Epilepsies (IGE/GGE); Temporal Lobe Epilepsy; Myoclonic Astatic Epilepsy (MAE); Migrating Partial Epilepsy of Infancy (MMPSI); and familial hemiplegic migraines, with or without epilepsy. In certain embodiments, the SCN2A-related disorder is late seizure onset epileptic encephalopathy. In certain embodiments, the SCN2A-related disorder is Benign Familial Neonatal-Infantile Seizures. In certain embodiments, the SCN2A-related disorder is an intellectual disability (ID). In certain embodiments, the SCN2A-related disorder is an autism spectrum disorder (ASD).

The methods of treatment disclosed herein may be used to ameliorate one or more symptoms of SCN2A disorders, including seizures, hypotonia, sensory issues, such as sensory integration disorders, motor development delays and dysfunctions, intellectual and cognitive dysfunctions, movement and balance dysfunctions, visual dysfunctions, delayed language and speech, gastrointestinal disorders, neurodevelopmental delays, and sleep problems. Seizures include focal, clonic, tonic, and generalized tonic and clonic seizures, prolonged seizures (often lasting longer than 10 minutes), and frequent seizures.

In some embodiments, administering an oligomeric compound to a subject in need thereof in the context of the present disclosure results in a decrease in the frequency of seizures in the subject, as compared to the frequency of seizures in the subject prior to administering an oligomeric compound. In some embodiments, administering an oligomeric compound to a subject in need thereof in the context of the present disclosure results in a decrease of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75% or at least about 80% in the frequency of seizures in the subject, as compared to the frequency of seizures in the subject prior to administering an oligomeric compound. In some embodiments, the decrease in the frequency of seizures in the subject is observed at least 1 week, at least 2 weeks, at least 4 weeks, at least 6 weeks, at least 8 weeks, at least 10 weeks, at least 12 weeks, at least 14 weeks or at least 16 or more weeks after the start of the administration of an oligomeric compound. In other embodiments, the decrease in frequency of seizures is observed within 24 hours after the start of administration of an oligomeric compound.

Interictal epileptiform discharges (IEDs) are abnormal electrical brain activity patterns that occur between seizures in patients with epilepsy (Smith et al., Elife. 2022 Jan. 20; 11:e73541. doi: 10.7554/eLife.73541. PMID: 35050851; PMCID: PMC8813051). It has been reported that IEDs in children with idiopathic epilepsy can affect both cognitive function and academic performance. (Cheng et al., BMC Neurol. 2020 Jun. 6; 20(1):233. doi: 10.1186/s12883-020-01807-z. PMID: 32505173; PMCID: PMC7275426). IEDs can be measured non-invasively using electroencephalography (EEG), a technique that records electrical activity in the brain through electrodes placed on the scalp. This makes it possible to monitor changes in IED frequency in real-time without causing discomfort to the subject.

Without wishing to be bound by a specific theory, it is believed that IEDs may be used clinically, e.g., to test effects of various therapies in subjects with DEEs. For example, LEDs can serve as an objective biomarker for the presence and severity of epileptic activity. By measuring the frequency and distribution in the brain of IEDs before and after administering a drug treatment, the drug's effectiveness in reducing epileptic activity can be monitored. It is also believed that changes in IED frequency and distribution may be detectable before any significant changes in seizure frequency or other clinical signs. Thus, IEDs can provide an early indication of the drug's effectiveness in treating DEE in a clinical trial, and may be used to inform target engagement and dosing strategies. It is also believed that a decrease in IED frequency after drug treatment may be associated with improved seizure control and cognitive outcomes. Thus, monitoring IEDs during a clinical trial can provide valuable information on the potential long-term benefits of the treatment.

In some embodiments, administering an oligomeric compound to a subject in need thereof in the context of the present disclosure results in a decrease in the frequency of IEDs in the subject, as compared to the frequency of IEDs in the subject prior to administering an oligomeric compound. In some embodiments, administering an oligomeric compound to a subject in need thereof in the context of the present disclosure results in a decrease of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75% or at least about 80% in frequency of IEDs in the subject, as compared to the frequency of IEDs in the subject prior to administering an oligomeric compound. In some embodiments, the decrease in the frequency of IEDs in the subject is observed at least 1 week, at least 2 weeks, at least 3 weeks, at least 4 weeks, at least 6 weeks, at least 8 weeks, at least 10 weeks, at least 12 weeks, at least 14 weeks or at least 16 or more weeks after the start of the administration of an oligomeric compound. In some embodiments, administering an oligomeric compound to a subject in need thereof in the context of the present disclosure results in a decrease in the frequency of amplitude-integrated electroencephalography (aEEG) signals, as compared to the frequency of aEEG signals in the subject prior to administering an oligomeric compound. In some embodiments, administering an oligomeric compound to a subject in need thereof in the context of the present disclosure results in a decrease of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75% or at least about 80% in frequency of aEEG signals in the subject, as compared to the frequency of aEEG signals in the subject prior to administering an oligomeric compound. In some embodiments, the decrease in the frequency of aEEG signals in the subject is observed at least 1 week, at least 2 weeks, at least 3 weeks, at least 4 weeks, at least 6 weeks, at least 8 weeks, at least 10 weeks, at least 12 weeks, at least 14 weeks or at least 16 or more weeks after the start of the administration of an oligomeric compound. In other embodiments, the decrease in frequency of aEEG signals in a subject is observed within 24 hours after the start of administration of an oligomeric compound.

In some aspects, the present disclosure also provides a method for evaluating efficacy of therapy for early onset developmental and epileptic encephalopathy (early onset DEE therapy), the method comprising measuring frequency of interictal epileptiform discharges (IEDs) in a subject being administered the early onset DEE therapy. In some embodiments, the method further comprises comparing frequency of IEDs in the subject measured prior to administration of the early onset DEE therapy to the frequency of IEDs in the subject measured after the start of administration of the early onset DEE therapy, wherein a decrease in the frequency of IEDs measured after the start of administration of the early onset DEE therapy is indicative that the early onset DEE therapy is effective and wherein lack of a decrease of an increase in the frequency of IEDs measured after the start of administration of the early onset DEE therapy is indicative that the early onset DEE therapy is not effective. In some embodiments, the early onset DEE therapy comprises an oligomeric compound.

In one aspect, the oligomeric compound useful in the methods of the present disclosure comprises a modified oligonucleotide consisting of 12 to 30 linked nucleosides wherein the nucleobase sequence of the modified oligonucleotide is at least 90% complementary to an equal length portion of an SCN2A nucleic acid, and wherein the modified oligonucleotide comprises at least one modification selected from a modified sugar moiety and a modified internucleoside linkage.

In certain embodiments, the oligomeric compound is at least 90% complementary to an equal length portion of SEQ ID NO: 2 and is not more than 50% complementary to an equal length portion of SEQ ID NO: 1.

In certain embodiments, the oligomeric compound has a nucleobase sequence comprising at least 12, at least 13, at least 14, at least 15, at least 16, at least 17, at least 18, at least 19, or 20 contiguous nucleobases of any of the nucleobase sequences of SEQ ID NOs: 16-2531.

In certain embodiments, the oligomeric compound has a nucleobase sequence comprising at least 12, at least 13, at least 14, at least 15, at least 16, at least 17, or 18 contiguous nucleobases of any of the nucleobase sequences of SEQ ID NOs: 2532-2539.

In certain embodiments, the oligomeric compound has a nucleobase sequence comprising:
a) at least 8, at least 9, at least 10, at least 11, at least 12, at least 13, at least 14, at least 15, at least 16, at least 17, at least 18, at least 19, or at least 20 contiguous nucleobases complementary to an equal length portion of nucleobases 199863-199905, 227493-22755, 243124-243204, 247823-247921, 254142-254177, 168911-168945, 170026-170061, 183519-183562, 188630-188668, 199912-199962, 227419-227450, or 238173-238192 of SEQ ID NO: 2, provided that the modified oligonucleotide does not comprise more than six LNA nucleosides; or
b) at least 8, at least 9, at least 10, at least 11, at least 12, at least 13, at least 14, at least 15, at least 16, at least 17, at least 18, at least 19, or at least 20 contiguous nucleobases complementary to an equal length portion of nucleobases 243917-244073, 170174-170200, 176724-176751, 180772-180801, 183968-184016, 202877-202906, 224198-224217, 224199-224218, or 243918-243937 of SEQ ID NO: 2.

In certain embodiments, the oligomeric compound has a nucleobase sequence comprising:
a) at least 8, at least 9, at least 10, at least 11, at least 12, at least 13, at least 14, at least 15, at least 16, at least 17, at least 18, at least 19, or at least 20 contiguous nucleobases of a sequence selected from SEQ ID NOs: 336, 488, 2021, 2097, 2174, 2250, 2326, 2403, 2499, 2500, 2501, 2502, 2526; 181, 259, 643, 720, 796, 2504, 2505, 2506, 2507, 2508, 2509, 2510, 2511, 2512, 2513, 2514, 2521; 491, 567, 644, 721, 797, 2177, 2253, 2315, 2329, 2406, 2527; 29, 30, 107, 108, 185, 186, 263, 264, 341, 342, 419, 420, 1796, 1871, 1948, 2025, 2101, 2178, 2254, 2330, 2503, 2517, 2522; 1016, 1093, 1104, 1169, 1246, 1323, 1400, 1477, 1554, 1708, 1785, 1860, 1937, 2014, 1631, 2090, 2539; 18, 96, 485, 561, 638, 715, 791, 868, 2247, 2323, 2400; 174, 1328, 1405, 1482, 1559, 1636, 1713, 1790, 1865, 1942, 2019; 20, 98, 253, 332, 410, 1406, 1483, 1560, 1637, 1714, 1791, 1866, 1943; 21, 411, 1407, 1484, 1561, 1638, 1715; 24, 414, 871, 948, 1025, 1100; 25, 337, 415, 490, 566, 2099, 2176, 2252, 2328, 2405; and 182; provided that the modified oligonucleotide does not comprise more than six LNA nucleosides; or
b) at least 8, at least 9, at least 10, at least 11, at least 12, at least 13, at least 14, at least 15, at least 16, at least 17, at least 18, at least 19, or at least 20 contiguous nucleobases of a sequence selected from SEQ ID NOs: 1090, 1166, 2484, 2485, 2487, 2493, 2496, 2497, 2498, 2533, 2534, 2535, 2537, 302, 1513, 1667, 1744, 1819, 1896, 197, 148, 226, 1364, 1441, 1518, 1595, 1672, 1749, 227, 1292, 1369, 1446, 1523, 1600, 1677, 1754, 1829, 228, 1679, 1756, 1831, 1908, 1985, 2061, 2138, 2214, 2290, 1226, 1303, 1380, 1457, 1534, 1611; 2079, 2523, and 2477.

In certain embodiments, the oligomeric compound has a nucleobase sequence comprising at least 12, at least 13, at least 14, at least 15, at least 16, at least 17, at least 18, at least 19, or 20 contiguous nucleobases of any of SEQ ID NOs: 2487, 2493, 2510, or 2514. In certain embodiments, the oligomeric compound has a nucleobase sequence comprising at least 12, at least 13, at least 14, at least 15, at least 16, at least 17, at least 18, at least 19, or 20 contiguous nucleobases of SEQ ID NO: 2510. In certain embodiments, the oligomeric compound comprises or consists of the nucleobase sequence of SEQ ID NO: 2510.

In certain embodiments, the oligomeric compound has a nucleobase sequence consisting of 17-19 or 21-30 linked nucleosides. In certain embodiments, the oligomeric compound has a nucleobase sequence consisting of 16, 17, 18, 19, or 20 linked nucleosides.

In certain embodiments, the oligomeric compound is an antisense oligonucleotide and comprises one or more modified sugar moieties, one or more modified internucleoside linkages, and one or more modified nucleobases, as described herein.

In certain embodiments, each of the one or more modified sugars is independently chosen from a bicyclic sugar, a 2′-O-methoxyethyl (2MOE) modified sugar, a 2′-O-methyl (2-OMe) modified sugar, a 2′-methoxy modified sugar, a 2′-Fluoro modified sugar, a 2′-O-alkyl modified sugar, a constrained ethyl (cEt) modified sugar, a locked sugar, or an unlocked sugar. In certain embodiments, the antisense oligonucleotide comprises one or more 2MOE modified sugars. In certain embodiments, the antisense oligonucleotide does not comprise a bicyclic sugar moiety.

In certain embodiments, each of one or more modified internucleoside linkages is independently chosen from a phosphorothioate, a phosphorodithioate, a phosphoramidate, a phosphorodiamidate, a thiophosphoramidate, a thiophosphorodiamidate, a methyl phosphonate, a phosphoromorpholidate, or a phosphoropiperazidate. In certain embodiments, each modified internucleoside linkage is a phosphorothioate internucleoside linkage. In certain embodiments, at least 10, at least 11, at least 12, at least 13, at least 14, at least 15, at least 16, at least 17, or at least 18 internucleoside linkages of the antisense oligonucleotide are phosphorothioate internucleoside linkages. In certain embodiments, each internucleoside linkage of the antisense oligonucleotide is independently selected from a phosphodiester or a phosphorothioate internucleoside linkage.

In certain embodiments, the internucleoside linkage motif of the modified oligonucleotide is selected from soooossssssssssooss, sooooossssssssssoss, sooossssssssssoooss, soosssssssssoooss, soooosssssssssoss, and sooosssssssssooss, wherein s=a phosphorothioate internucleoside linkage and o=a phosphodiester internucleoside linkage.

In certain embodiments, the one or more modified nucleobases is chosen from 5-methylcytosine, 5-hydroxymethyl cytosine, xanthine, hypoxanthine, 2-aminoadenine, 6-methyladenine, 6-methylguanine, 2-propyladenine, 2-propylguanine, 2-thiouracil, 2-thiothymine, 2-thiocytosine, 5-halouracil, 5-halocytosine, 5-propynyluracil, 5-propynylcytosine, 6-azouracil, 6-azocytosine, 6-azothymine, 5-uracil (pseudouracil), 4-thiouracil, 8-haloadenine, 8-aminoadenine, 8-thioladenine, 8-thioalkyladenine, 8-hydroxyladenine, 8-haloguanine, 8-aminoguanine, 8-thiolguanine, 8-thioalkylguanine, 8-hydroxylguanine, 5-bromouracil, 5-trifluoromethyluracil, 5-bromocytosine, 5-trifluoromethylcytosine, 7-methylguanine, 7-methyladenine, 2-fluoroadenine, 8-azaguanine, 8-azaadenine, 7-deazaguanine, 7-deazaadenine, 3-deazaguanine, or 3-deazaadenine.

In certain embodiments, the modified nucleobase is a 5-methylcytosine. In certain embodiments, each cytosine in the antisense oligonucleotide is a 5-methylcytosine.

In certain embodiments, the antisense oligonucleotide comprises: a gap segment consisting of linked deoxynucleosides; a 5′ wing segment consisting of linked nucleosides; and a 3′ wing segment consisting of linked nucleosides; wherein the gap segment is positioned immediately adjacent to and between the 5′ wing segment and the 3′ wing segment and wherein each nucleoside of each wing segment comprises a modified sugar.

In certain embodiments, the antisense oligonucleotide does not comprise a bicyclic sugar moiety, the antisense oligonucleotide is characterized according to the following chemical notation: Gₑₛ^{m}CₑₒAₑₒTₑₒAₑₒA_{ds}T_{ds}^{m}C_{ds}^{m}C_{ds}^{m}C_{ds}A_{ds}T_{ds}T_{ds}A_{ds}T_{ds}Aₑₒ^{m}CₑₒAₑₛAₑₛAₑ (SEQ ID NO: 2493), wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

In certain embodiments, the antisense oligonucleotide is characterized according to the following chemical notation:

(SEQ ID NO: 2514)
^{m}CₑₛAₑₒ^{m}CₑₒGₑₒAₑₒ^{m}CₑₒA_{ds}T_{ds}A_{ds}T_{ds}T_{ds}
T_{ds}T_{ds}T_{ds}^{m}C_{ds}TₑₛAₑₒ^{m}CₑₛAₑₛ^{m}Cₑ,

wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

In certain embodiments, the antisense oligonucleotide is characterized according to the following chemical notation:

(SEQ ID NO: 2510)
^{m}Cₑₛ^{m}CₑₒAₑₒ^{m}C_{deo}GₑₒAₑₒ^{m}C_{ds}A_{ds}T_{ds}A_{ds}
T_{ds}T_{ds}T_{ds}T_{ds}T_{ds}^{m}C_{ds}TₑₒAₑₛ^{m}CₑₛAₑ,

wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

In certain embodiments, the antisense oligonucleotide is characterized according to the following chemical notation:

(SEQ ID NO: 2487)
Tₑₛ^{m}CₑₒTₑₒGₑₒ^{m}CₑₒAₑₒT_{ds}G_{ds}T_{ds}A_{ds}A_{ds}^{m}C_{ds}
^{m}C_{ds}T_{ds}T_{ds}T_{ds}AₑₒTₑₛAₑₛ^{m}Cₑ,

wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

In certain embodiments, the antisense oligonucleotide is characterized according to the following chemical notation:

(SEQ ID NO: 2493)
Gₑₛ^{m}CₑₒAₑₒTₑₒAₑₒAₑₒT_{ds}^{m}C_{ds}^{m}C_{ds}^{m}C_{ds}A_{ds}
T_{ds}T_{ds}A_{ds}T_{ds}A_{ds}^{m}CₑₒAₑₛAₑₛAₑ,

wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

In certain embodiments, the antisense oligonucleotide is characterized according to the following chemical notation:

(SEQ ID NO: 2534)
GT^{m}CₑₛTₑₒGₑₒ^{m}CₑₒAₑₛT_{ds}G_{ds}T_{ds}A_{ds}
A_{ds}^{m}C_{ds}^{m}C_{ds}T_{ds}TₑₒTₑₒAₑₛTₑₛAₑ,

wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothinate internucleoside linkage, and
o=a phosphodiester internucleoside linkage.

In certain embodiments, the antisense oligonucleotide has the following chemical structure:

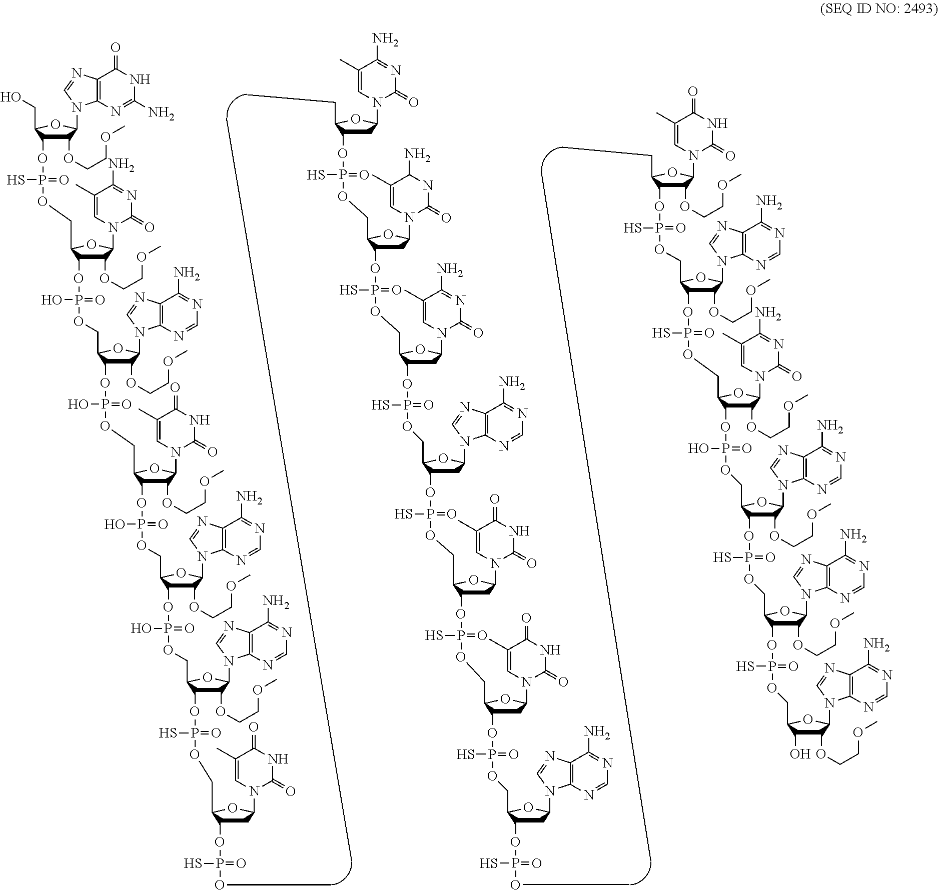

or a salt thereof, such as a potassium salt.

In certain embodiments, the antisense oligonucleotide has the following chemical structure:

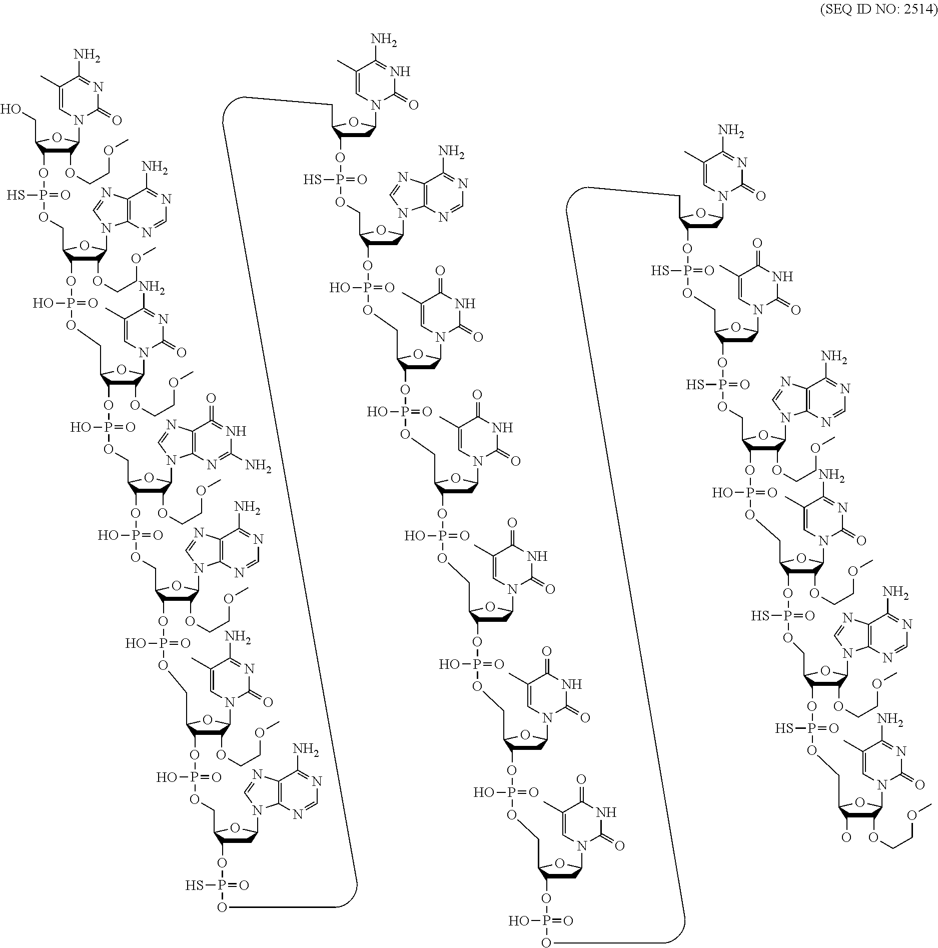

or a salt thereof, such as a potassium salt

In certain embodiments, the antisense oligonucleotide has the following chemical structure:

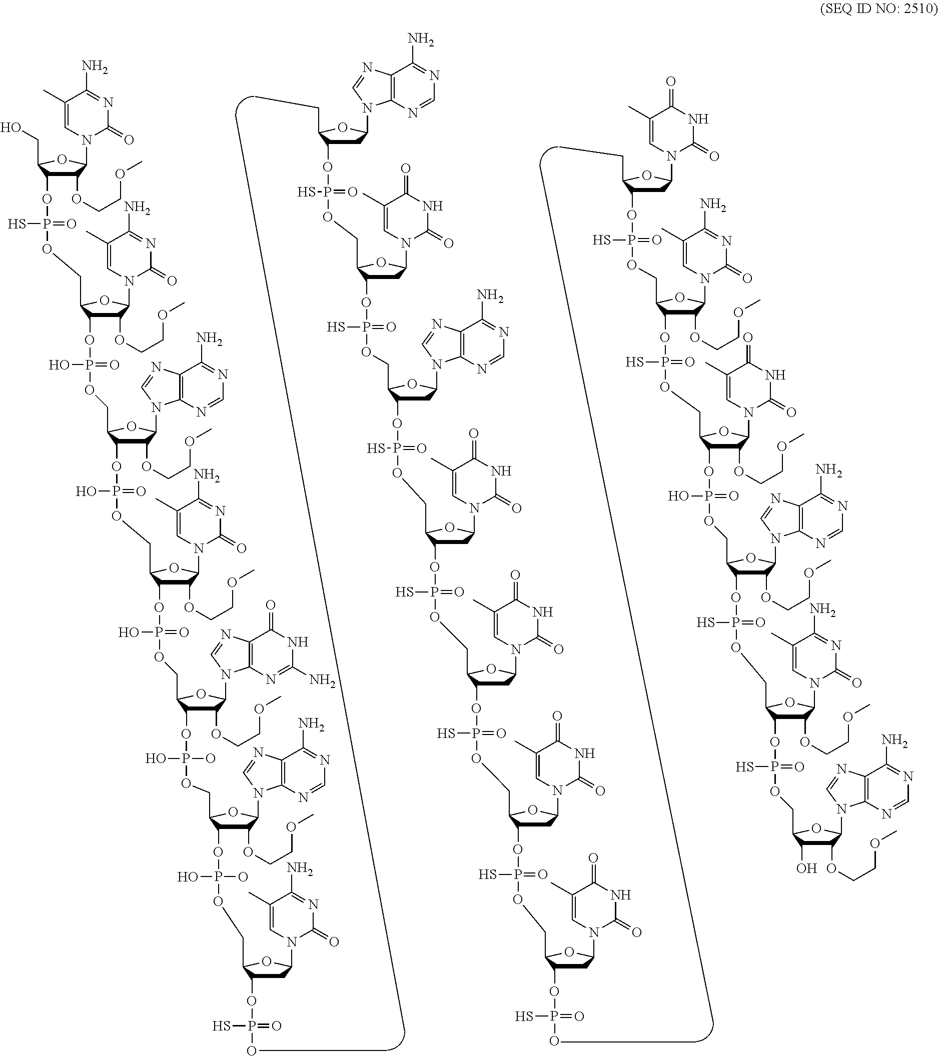

or a salt thereof, such as a potassium salt.

In certain embodiments, the antisense oligonucleotide has the following chemical structure:

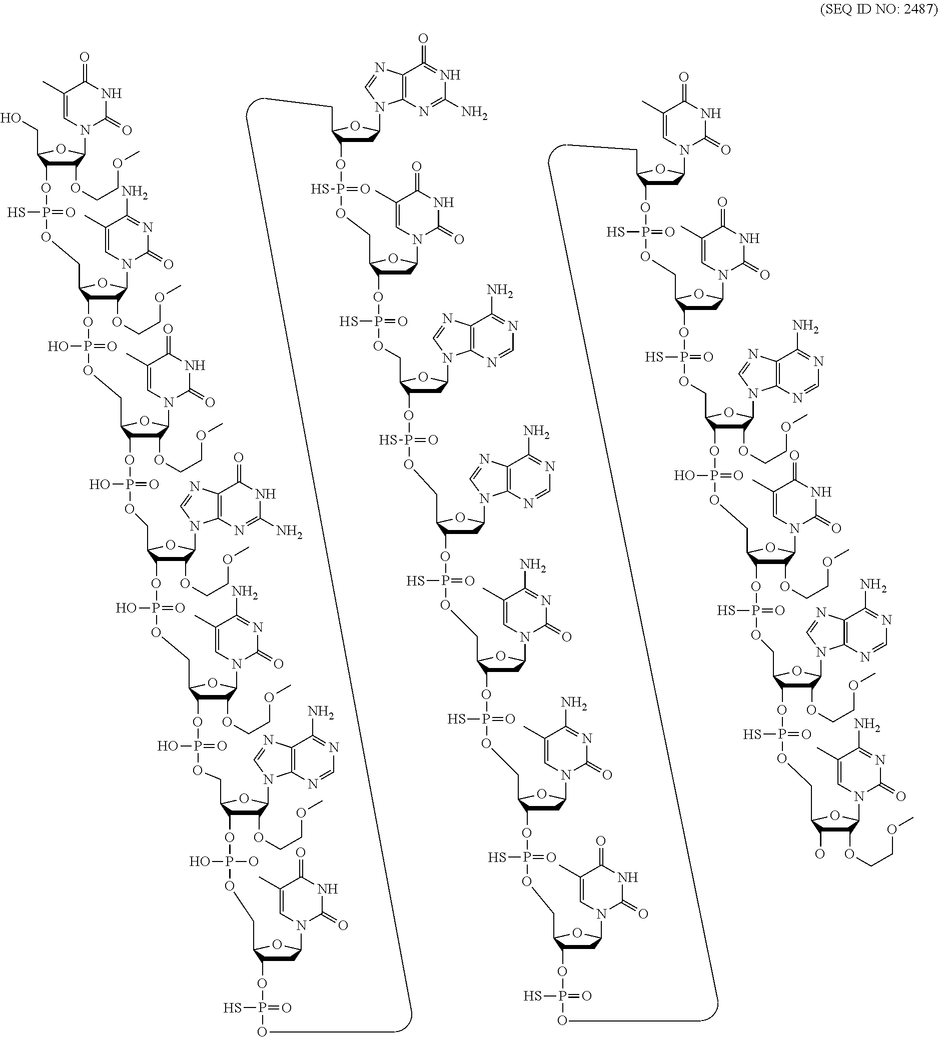

or a salt thereof, such as a potassium salt.

In certain embodiments, the antisense oligonucleotide has the following chemical structure:

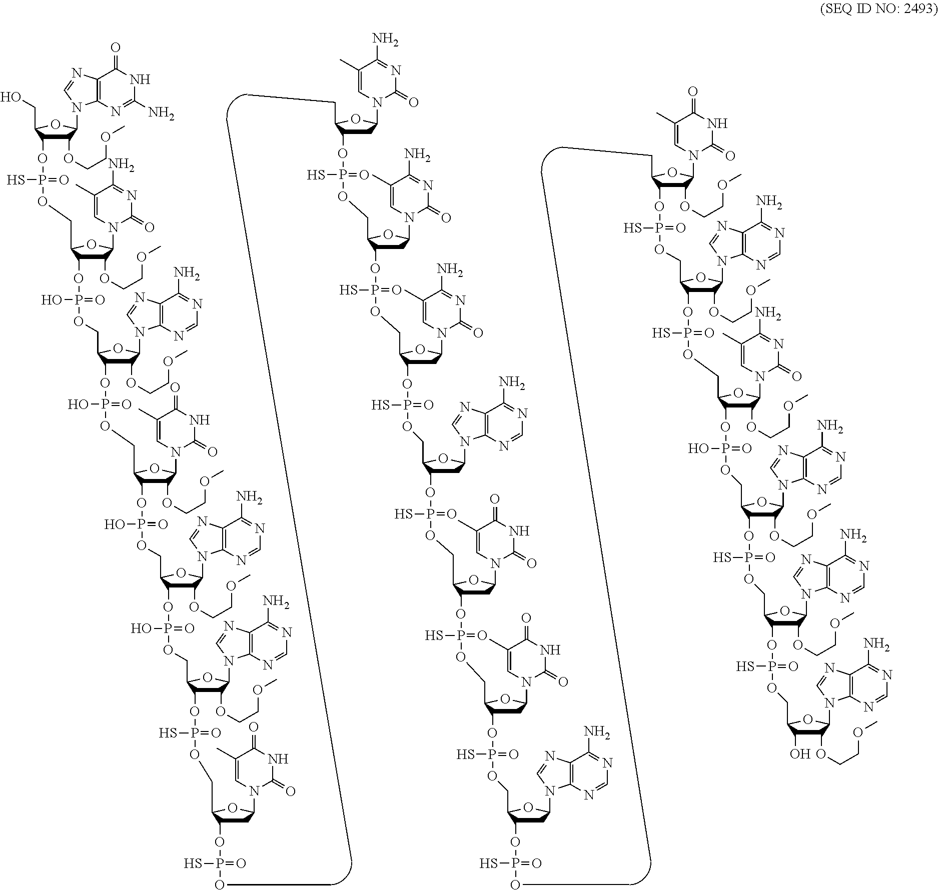

or a salt thereof, such as a potassium salt.

In certain embodiments, the antisense oligonucleotide has the following chemical structure:

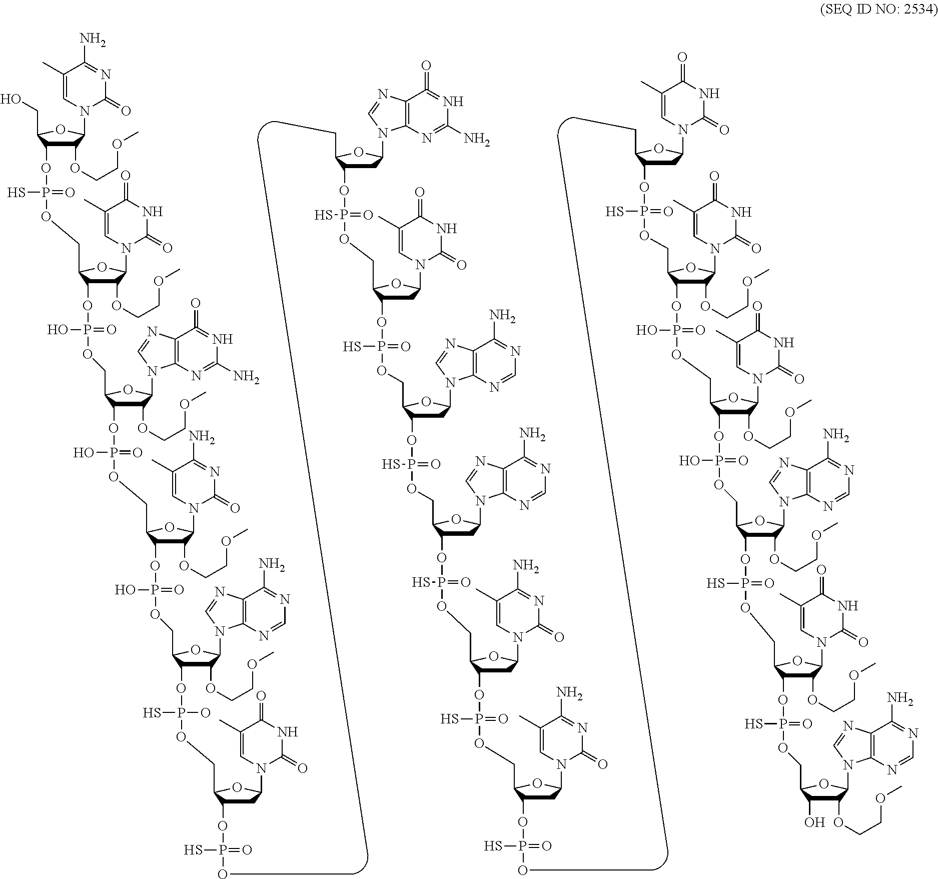

### EXAMPLES

### Example 1. A Seamless, Clinical Trial to Investigate the Safety and Efficacy of Multiple Doses of Compound 1 in Pediatric Participants with Early Onset SCN2A Developmental and Epileptic Encephalopathy

This is a seamless, in part non-randomized, open label, and in part randomized, placebo procedure-controlled double-blind, clinical trial to explore the safety, tolerability, P, and efficacy of ascending doses of an oligomeric compound of the present disclosure (COMPOUND 1) in pediatric participants with early onset SCN2A DEE, aged 2 to 18 years. The trial will be conducted in 4 parts: the preliminary safety Part a (open label), dose-escalation Part A (double blind), confirmatory Part B (double blind), followed by an open-label extension in Part C.

### Objectives and Endpoints

### Part 1: Preliminary Safety

| | |
|---|---|
| Objective | Endpoint |

Primary

| | |
|---|---|
| To evaluate the safety and tolerability of | Incidence and severity of treatment- |
| COMPOUND 1 administered by | emergent adverse events (TEAEs) |
| intrathecal (IT) injection in participants | Changes in findings on physical and |
| with early onset SCN2A DEE | neurological examinations |
| | Changes in video electroencephalogram |
| | (vEEG) characteristics as determined by a |
| | central reader |
| | Changes in vital sign measurements |
| | Changes in clinical laboratory results |
| | Changes in electrocardiogram (ECG) |
| | parameters |

Secondary

| | |
|---|---|
| To explore the preliminary efficacy of | Seizure frequency per 28 days, as assessed |
| COMPOUND 1 in participants with early | by seizure diary, over the time period after |
| onset SCN2A DEE | the 4^{th} dose administration |
| | Seizure frequency per 28 days over the time |
| | period immediately after each dose |
| | administration |
| | Percent change from baseline in seizure |
| | frequency per 28 days over the time period |
| | immediately after each dose administration |
| | Response, defined as ≥50% reduction in |
| | seizure frequency per 28 days over the time |
| | period immediately after each dose |
| | administration |

PK

| | |
|---|---|
| To characterize the pharmacokinetics (PK) | Plasma and cerebrospinal fluid (CSF) |
| of COMPOUND 1 | concentrations of COMPOUND 1 |
| | Plasma COMPOUND 1 PK parameters |

Exploratory

| | |
|---|---|
| To explore additional preliminary efficacy | Electroencephalogram (EEG) |
| of COMPOUND 1 | characteristics, including but not limited to |
| | electrographic seizures, interictal |
| | epileptiform discharges, background |
| | frequency, and features of sleep at each post |
| | dose timepoint |
| To evaluate the impact of COMPOUND 1 | Levels of inflammatory markers (including |
| on inflammatory markers | C-reactive protein [CRP] and complement |
| | [test for total complement activity - CH50, |
| | C3a, Bb and C5a], fibrinogen) |
| To evaluate the potential for | Presence of anti-drug antibodies (ADA) in |
| immunogenicity towards COMPOUND 1 | plasma |

### Part a: Dose Escalation

| | |
|---|---|
| Objective | Endpoint |

Primary

| | |
|---|---|
| To evaluate the safety and tolerability of | Incidence and severity of TEAEs |
| ascending doses of COMPOUND 1 | Changes in findings on physical and |
| administered by intrathecal (IT) injection | neurological examinations |
| in participants with early onset SCN2A | Changes in vEEG characteristics as |
| DEE | determined by a central reader |
| | Changes in vital sign measurements |
| | Changes in clinical laboratory results |
| | Changes in ECG parameters |

Secondary

| | |
|---|---|
| To explore the preliminary efficacy of | Seizure frequency per 28 days, as assessed |
| COMPOUND 1 compared with placebo | by seizure diary, over the time period after |
| in participants with early onset SCN2A | the 6^{th} dose administration |
| DEE | Seizure frequency per 28 days over the time |
| | period immediately after each dose |
| | administration |
| | Percent change from baseline in seizure |
| | frequency per 28 days over the time period |
| | immediately after each dose administration |
| | Response, defined as ≥50% reduction in |
| | seizure frequency per 28 days over the time |
| | period immediately after each dose |
| | administration |

PK

| | |
|---|---|
| To characterize the PK of COMPOUND 1 | Plasma and CSF concentrations of |
| | COMPOUND 1 |
| | Plasma COMPOUND 1 PK parameters |

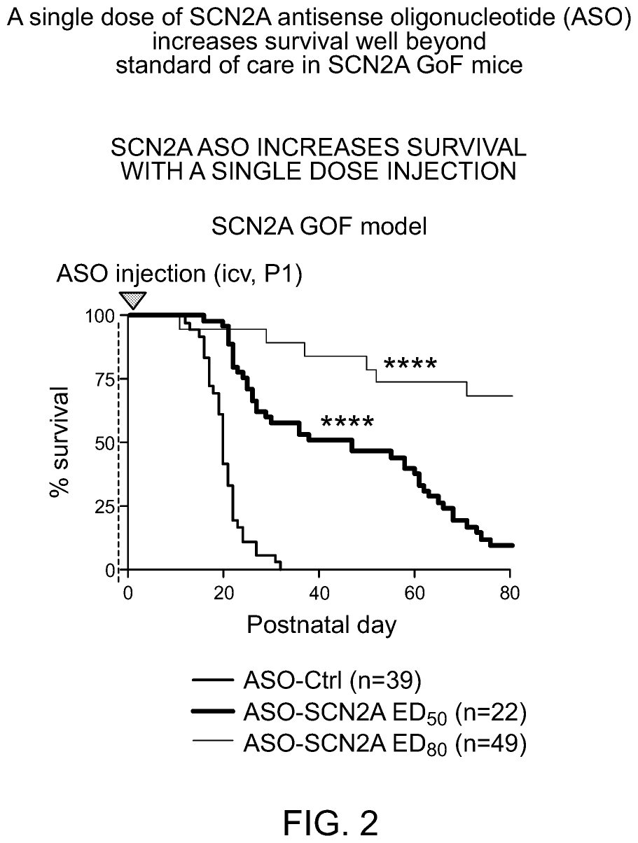

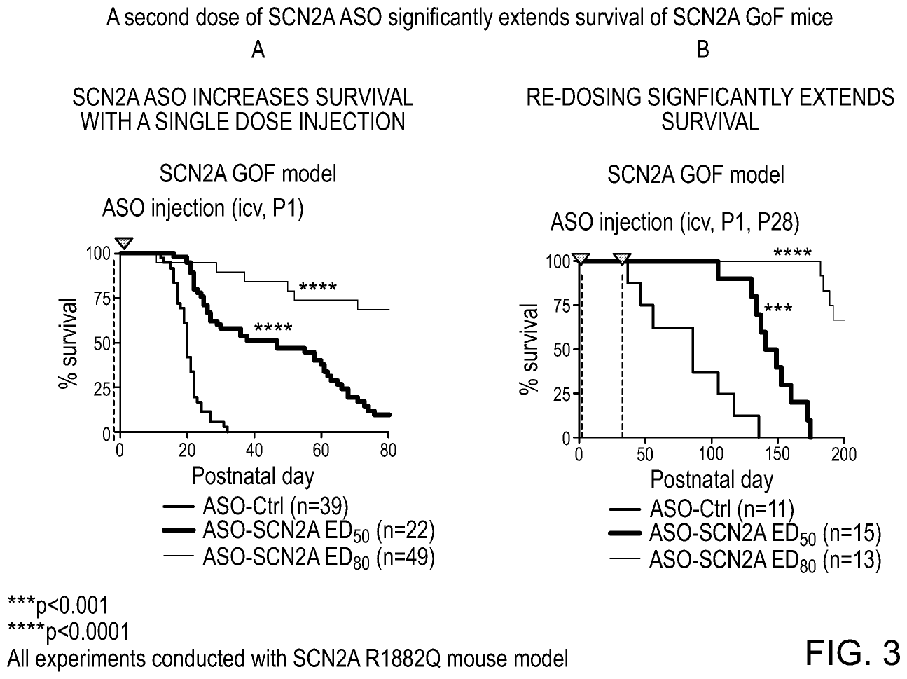

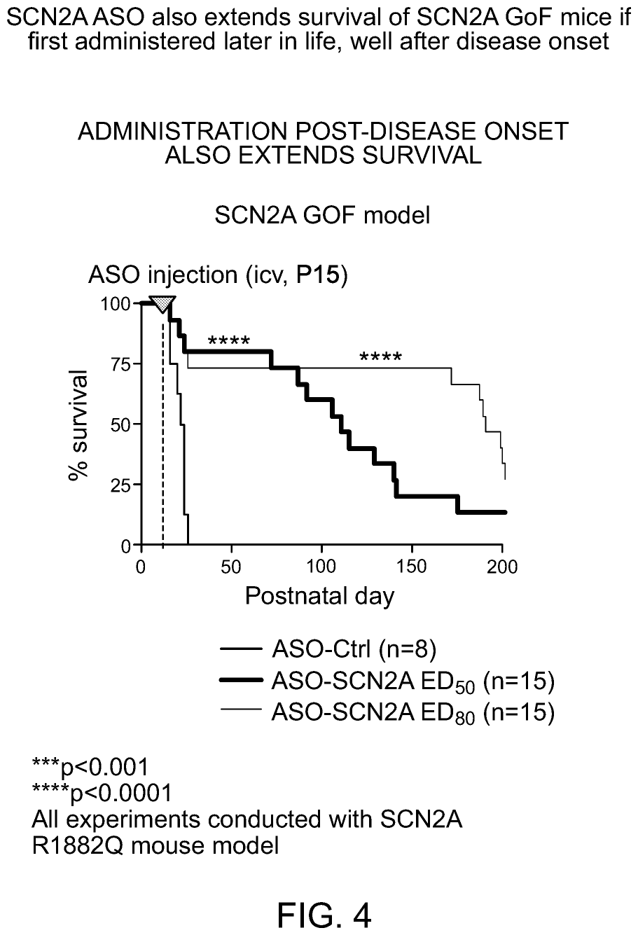

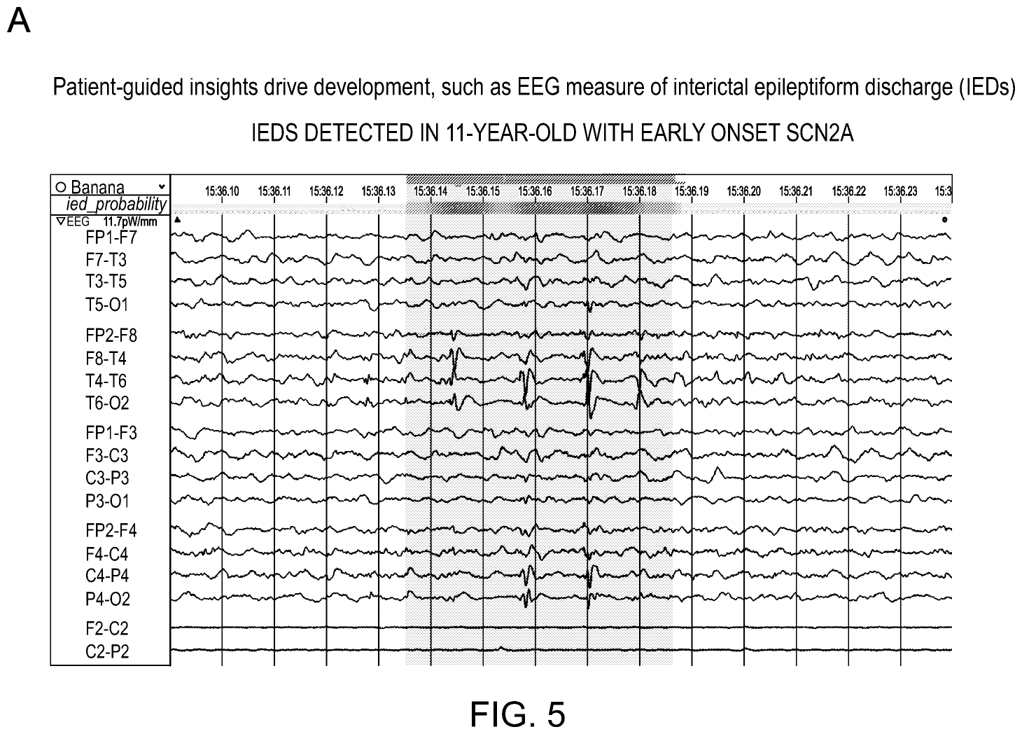

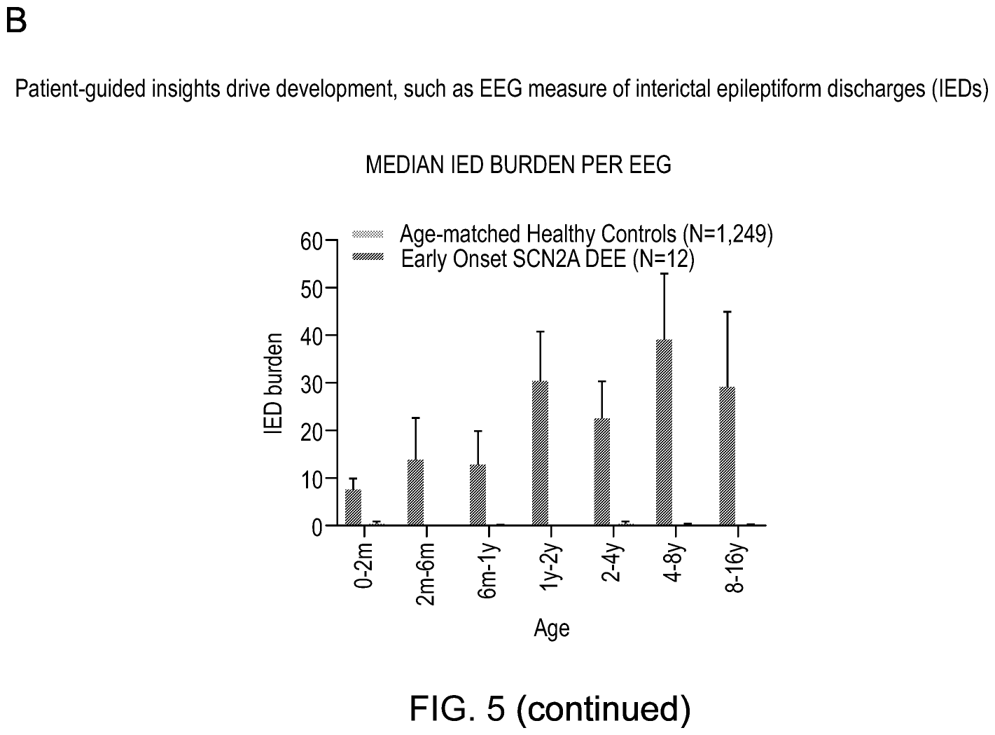

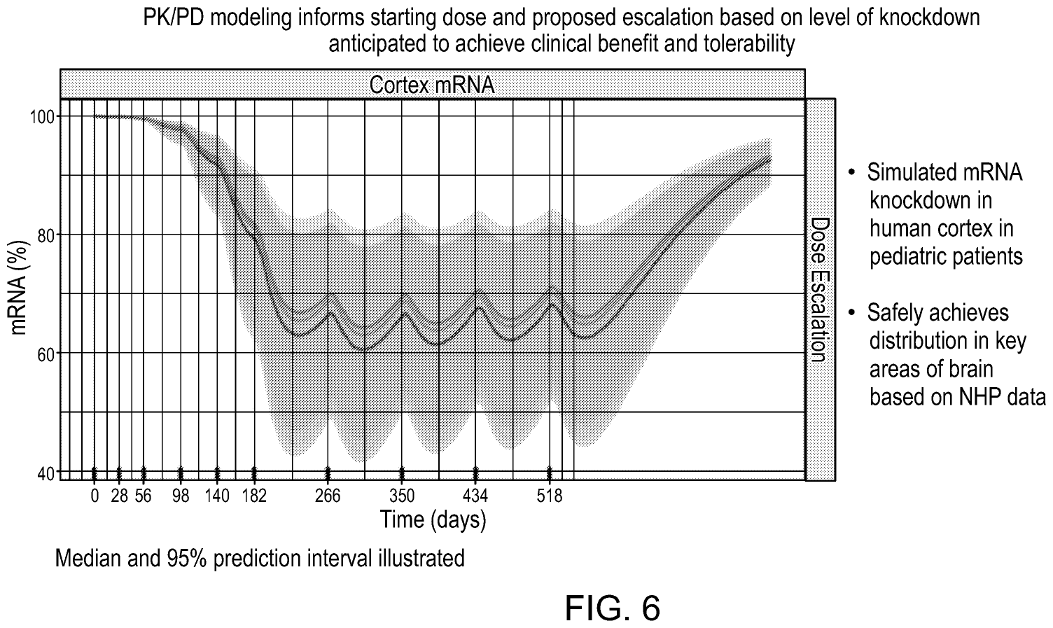

Exploratory

| | |
|---|---|
| To explore additional preliminary efficacy | EEG characteristics, including but not |
| of COMPOUND 1 compared with | limited to electrographic seizures, interictal |
| placebo | epileptiform discharges, background |
| | frequency, and features of sleep at each post |
| | dose timepoint |
| | Clinical Global Impression-Severity (CGI- |
| | S) at baseline and Clinical Global |
| | Impression-Improvement (CGI-I) scores at |
| | each post dose timepoint |
| | Caregiver Global Impression-Severity |
| | (CgGI-S) at baseline and Caregiver Global |
| | Impression-Improvement (CgGI-I) scores at |
| | each post dose timepoint |
| | Quality of life as assessed by Quality of |
| | Life Inventory-Disability (QI-Disability) at |
| | the end of Part A |
| | Developmental milestones as assessed by |
| | Bayley Scales of Infant Development - |
| | Fourth Edition (Bayley-4) domain and |
| | subtest scores or Wechsler Preschool and |
| | Primary Scales of Intelligence, fourth |
| | edition (WPPSI IV), depending on the |
| | specific cognitive test the participant is |
| | assigned to as shown in FIG. 2, at the end |
| | of Part A |
| | Behavior as assessed by Vineland Adaptive |
| | Behavior Scale-3 (Vineland-3) domain and |
| | subdomain scores at end of Part A |
| | Behavior as assessed by Aberrant Behavior |
| | Checklist - 2^{nd} Edition (ABC-2) total and |
| | subscale scores at the end of Part A |
| | Sleep as assessed by Sleep Disturbance |
| | Scale for Children at the end of Part A |
| To evaluate the impact of COMPOUND 1 | Levels of inflammatory markers (including |
| on inflammatory markers | CRP and complement [CH50, C3a, Bb and |
| | C5a], fibrinogen) |
| To evaluate the potential for | Presence of ADA in plasma |
| immunogenicity towards COMPOUND 1 | |

### Part B: Confirmatory

| | |
|---|---|
| Objective | Endpoint |

Primary

| | |
|---|---|
| To assess the efficacy of COMPOUND | Seizure frequency per 28 days, as |
| 1 administered by IT injections | assessed by seizure diary, over the time |
| compared with placebo in participants | period after the 6^{th} dose administration |
| with early onset SCN2A DEE | |

Secondary

| | |
|---|---|
| To assess the secondary efficacy of | Seizure frequency per 28 days during the |
| COMPOUND 1 administered by IT | time period immediately after each dose |
| injections compared with placebo in | administration |
| participants with early onset SCN2A | Percent change from baseline in seizure |
| DEE | frequency per 28 days over the time |
| | period immediately after each dose |
| | administration |
| | Response, defined as ≥50% reduction in |
| | seizure frequency per 28 days, over the |
| | time period immediately after each dose |
| | administration |
| | EEG characteristics, including but not |
| | limited to electrographic seizures, |
| | interictal epileptiform discharges, |
| | background frequency, and features of |
| | sleep at each post dose timepoint |
| | CGI-S at baseline and CGI-I scores at |
| | each post dose timepoint |
| | CgGI-S at baseline and CgGI-I scores at |
| | each post dose timepoint |
| | Quality of life as assessed by Quality of |
| | Life Inventory-Disability (QI-Disability) |
| | at the end of Part B |
| | Developmental milestones as assessed by |
| | Bayley Scales of Infant Development - |
| | Fourth Edition (Bayley-4) domain and |
| | subtest scores or WPPSI IV, depending |
| | on the specific cognitive test the |
| | participant is assigned to at the end of |
| | Part B |
| | Behavior as assessed by Vineland |
| | Adaptive Behavior Scale-3 (Vineland-3) |
| | domain and subdomain scores at the end |
| | of Part B |
| | Behavior as assessed by ABC-2 total and |
| | subscale scores at the end of Part B |
| | Sleep as assessed by Sleep Disturbance |
| | Scale for Children at the end of Part B |

Safety

| | |
|---|---|
| To evaluate the safety and tolerability of | Incidence and severity of TEAEs |
| COMPOUND 1 administered by IT | Changes in findings on physical and |
| injection in participants with early onset | neurological examinations |
| SCN2A DEE | Changes in vEEG characteristics as |
| | determined by a central reader |
| | Changes in vital sign measurements |
| | Changes in clinical laboratory results |
| | Changes in ECG parameters |

PK

| | |
|---|---|
| To characterize the PK of COMPOUND | Plasma and CSF concentrations of |
| 1 | COMPOUND 1 |
| | Plasma COMPOUND 1 PK parameters |

Exploratory

| | |
|---|---|
| To evaluate the impact of COMPOUND | Levels of inflammatory markers |
| 1 on inflammatory markers | (including CRP and complement [CH50, |
| | C3a, Bb and C5a], fibrinogen) |
| To evaluate the potential for | Presence of ADA in plasma |
| immunogenicity towards COMPOUND | |
| 1 | |

### Part C: Open-Label Extension

| | |
|---|---|
| Objective | Endpoint |

Exploratory

| | |
|---|---|
| To evaluate the durability of efficacy of | Seizure frequency per 28 days, as |
| COMPOUND 1 administered by IT | assessed by seizure diary, over the time |
| injections at a maintenance dosing | period immediately after each dose |
| schedule | administration |
| | Percent change from baseline in seizure |
| | frequency per 28 days over the time |
| | period immediately after each dose |
| | administration |
| | Response, defined as ≥50% reduction in |
| | seizure frequency per 28 days over the |
| | time period after each dose |
| | administration |
| | EEG characteristics, including but not |
| | limited to electrographic seizures, |
| | interictal epileptiform discharges, |
| | background frequency, and features of |
| | sleep at each post dose timepoint |
| | CGI-S at baseline and CGI-I scores at |
| | each post dose timepoint |
| | CgGI-S at baseline and CgGI-I scores at |
| | each post dose timepoint |
| | Quality of life as assessed by Quality of |
| | Life Inventory-Disability (QI-Disability) |
| | at the end of Part C |
| | Developmental milestones as assessed by |
| | Bayley Scales of Infant Development - |
| | Fourth Edition (Bayley-4) domain and |
| | subtest scores or WPPSI IV, depending |
| | on the specific cognitive test the |
| | participant is assigned to at the end of |
| | Part C |
| | Behavior as assessed by Vineland |
| | Adaptive Behavior Scale-3 (Vineland-3) |
| | domain and subdomain scores at the end |
| | of Part C |
| | Behavior as assessed by ABC-2 total and |
| | subscale scores at the end of Part C |
| | Sleep as assessed by Sleep Disturbance |
| | Scale for Children at the end of Part C |
| To evaluate the impact of COMPOUND | Levels of inflammatory markers |
| 1 on inflammatory markers | (including CRP and complement [CH50, |
| | C3a, Bb and C5a], fibrinogen) |
| To evaluate the potential for | Presence of ADA in plasma |
| immunogenicity towards COMPOUND | |
| 1 | |

Safety

| | |
|---|---|
| To evaluate the safety and tolerability of | Incidence and severity of TEAEs |
| COMPOUND 1 administered by IT | Changes in findings on physical and |
| injection during maintenance dosing in | neurological examinations |
| participants with early onset SCN2A | Changes in vEEG characteristics as |
| DEE | determined by a central reader |
| | Changes in vital sign measurements |
| | Changes in clinical laboratory results |
| | Changes in ECG parameters |

PK

| | |
|---|---|
| To characterize the PK of COMPOUND | Plasma and CSF concentrations of |
| 1 during maintenance dosing | COMPOUND 1 |
| | Plasma COMPOUND 1 PK parameters |

### Part 1: Preliminary Safety

Part 1 will be conducted exclusively in the United States (US) with the objective of obtaining clinical safety data which will further inform the dose escalation in Part A. In this open label, non-randomized part of the trial, 4 participants will be enrolled and receive COMPOUND 1 1 mg doses at ≥4-week intervals for up to 13 weeks.

Safety, along with available PK data, will be assessed by the sponsor on an ongoing basis along with scheduled quarterly Data Monitoring Committee (DMC) reviews after each interim analysis, and ad hoc reviews as needed based on emerging safety data. Preliminary efficacy in seizure reduction and safety will also be assessed after 4 doses.

After 4 participants receive a minimum of 2 doses of COMPOUND 1, and have had a minimum of 2 weeks monitoring thereafter, the collected safety, tolerability and PK data from this trial part will be submitted to the FDA for interim review. The trial will not proceed to Parts A, B and C until a favorable opinion and approval for the trial continuation is obtained from the agency.

Following their final dose, participants from Part 1 will have the potential to transition to long term extension, subject to the FDA's further review and approval of the trial continuation, and pending updated chronic toxicology data.

### Part A: Dose Escalation

Part A will initiate in the US, only after approval from regulatory authorities upon completion of Part 1. The goal of Part A is to identify a cumulative dose of COMPOUND 1 with a favorable benefit-risk profile for seizure reduction that can be further assessed in Part B. Part A will consist of up to 16 participants. Initially, 8 treatment-naïve participants will be randomized 3:1 to receive ascending doses of COMPOUND 1 or a placebo procedure. The first 4 participants must reach at least 4 weeks after their first dose before additional participants can be administered study drug. Administrations will occur at ≥4-week intervals for the first 4 doses, and at ≥6-week intervals for the subsequent doses. Dose escalations will be within-participant. The starting dose will be 1 mg with ≤2-fold increase—for subsequent doses with specific dose escalation criteria in Section 6.5. The target is to reach a cumulative dose of up to 42 mg, which is predicted to correspond to approximately 35% knock down (KD), divided into 6 administrations.

Safety, along with available PK data, will be assessed by the sponsor on an ongoing basis along with scheduled quarterly DMC reviews and ad hoc reviews as needed based on emerging safety data. Preliminary efficacy in seizure reduction and safety will be assessed during the time period after the 6^{th} dose administration after up to 8 participants have reached a cumulative dose of up to 42 mg or a corresponding placebo procedure.

The dosing regimen may be revised based on the analysis of safety and efficacy. Maximum tolerated dose (MTD) in this trial is defined as 1 dose level below a single or cumulative dose associated with 2 or more Dose Limiting Toxicities (DLTs) in Part A or is a 25% rate trial-wide. Up to 8 additional participants may be added (Group 2, randomized to active or placebo in a 3:1 ratio) to further assess the safety and efficacy of escalating doses of COMPOUND 1. The starting dose for these additional participants will be up to the highest tolerated single dose previously administered. A second planned analysis assessing safety and efficacy during the time period after the 6^{th} dose will occur after up to 8 participants have reached up to 100 mg cumulatively. After the final dose (or placebo procedure), participants will subsequently have the opportunity to enter Part C/open-label extension.

### Part B: Confirmatory Phase

Part B will further assess and confirm the safety and efficacy of the cumulative dose identified in Part A in a randomized, placebo procedure-controlled design. This trial part will only be initiated after authorization is obtained from regulatory authorities. Up to 40 treatment-naïve participants will be randomized, with no more than 75% to receive COMPOUND 1 over placebo treatment (3:1 randomization maximum). The final sample size and randomization scheme will be further confirmed based on the preliminary data and power calculations from Part A.

The cumulative dose and highest single dose will not exceed the respective MTD in Part A. The cumulative dose from Part A will be divided into ≤6 administrations of COMPOUND 1 or placebo procedure and will be given no more frequently than every 6 weeks.

The primary endpoint will be seizure frequency over the time period after the 6^{th} dose as assessed by the seizure diary. Thus, the duration of participation in Part B is up to 30 weeks. After the final dose (or placebo procedure) participants will subsequently have the opportunity to enter the open-label extension of the trial, Part C.

Parts 1, A, and B will each consist of 3 periods: Screening Period, Intervention Period (open-label in Part 1, double-blind in Parts A and B), and Follow-up Period.

### Screening Period

Prior to any clinical trial procedures, the participant/caregiver will provide written informed consent and satisfy inclusion/exclusion criteria. Key assessments during Screening will include medical and disease history, demographic data, body weight and height, physical examination (including detailed neurological examination), clinical laboratory evaluations, magnetic resonance imaging (MRI) of the brain, vital signs measurements, 12-lead ECGs, and a review of concomitant medications/procedures as outlined in the Schedule of Activities (SoA) (Table 1, Table 2, and Table 3 for Part 1, Part A, and Part B, respectively).

Caregivers will complete a daily seizure diary for at minimum 4 weeks during the Baseline Observation period (prior to Baseline Dosing Visit) through EOT, including the days without visits. Other screening assessments may still occur during this 4-week period (and do not necessarily have to be completed prior to the start of the daily seizure diary). Each participant's eligibility will be reviewed and approved by an Eligibility Review Committee (ERC) before they are included into the trial. A baseline prolonged vEEG will be completed within the 4 weeks prior to Baseline dosing visit.

### Intervention Period

Part 1 (Open-label Intervention): Participants will be admitted to the trial site on Day −1 for Baseline dosing visit. On Day 1, after confirmation of eligibility, they will receive COMPOUND 1 via IT administration. Each participant will remain inpatient for at least 24-48 hours after dosing for physical examination (including detailed neurological examination), clinical laboratory evaluations, vital signs measurements, 12-lead ECGs, and any other trial assessments in this visit as outlined in the SoA (Table 1). The corresponding procedures and assessments including COMPOUND 1 administration, will take place again in intervals of approximately (but not more frequent than) every 4 weeks, for up to 4 doses in total. Dosing in Part 1 may be less frequent based on the tolerability of the study drug.

Approximately 2 weeks after each P COMPOUND 1 administration, home-health visits, as well as home vEEG, will be conducted as per the SoA (Table 1). Home-health visits may be conducted at home (i.e., remote to the investigational site) or in the clinic (at the investigational site), at the discretion of the participant together with the investigator. If there are any concerning AEs, or changes in the participant's examination, the participant may return at the discretion of the investigator for an unscheduled visit in the clinic for further evaluation.

Parts A and B (Double-blind Interventions): Participants will be admitted to the trial site on Day −1 for Baseline dosing visit. On Day 1, after confirmation of eligibility, they will receive Compound 1 via IT administration, or placebo procedure per the randomization schedule. Each participant will remain inpatient for at least 24-48 hours after dosing for physical examination (including detailed neurological examination), clinical laboratory evaluations, vital signs measurements, 12-lead ECGs, and any other trial assessments in this visit as outlined in the SoA (Table 2, Table 3). The corresponding procedures and assessments including COMPOUND 1 administration, or placebo procedure, will take place again in intervals of approximately (but not more frequent than) every 4 weeks for the first 4 doses, and every 6 weeks for the subsequent doses in Part A, and every 6 weeks in Part B as outlined in the SoA (Table 2, Table 3).

Approximately 2 weeks after each COMPOUND 1 administration or placebo procedure, home-health visits, as well as home vEEG, will be conducted as per the SoA (Table 2, Table 3). Home-health visits may be conducted at home (i.e., remote to the investigational site) or in the clinic (at the investigational site), at the discretion of the participant together with the investigator. If there are any concerning AEs, or changes in the participant's examination, the participant may return at the discretion of the investigator for an unscheduled visit in the clinic for further evaluation.

### Safety Follow-Up Period

During the 6-month follow-up period, home-health visits, as well as home vEEG as outlined in the SoA (Table 1, Table 2, Table 3), will be conducted. Home-health visits may be conducted at home (i.e., remote to the investigational site) or in the clinic (at the investigational site), at the discretion of the participant together with the investigator. If there are any concerning Aes or changes in the participant's examination (per caregiver report or noted at the home-health visit), the participant may return at the discretion of the investigator for an unscheduled visit in the clinic for further evaluation. A final visit to the clinic will occur for the end of trial assessments as outlined in the SoAs (Table 1, Table 2, Table 3).

The follow-up period may be extended based on the results of the assessments at the end of study (EOS) visit and the investigator's best clinical judgement. Should it be considered necessary, the extended follow-up will include assessments for the presence of potential long-term and developmental effects.

### Part C: Open-Label Extension

Open label extension will be available for any patient with prior exposure to COMPOUND 1 or if they are rolling over from an active COMPOUND 1 trial.

### Following Part 1

Following their final dose, participants from Part 1, will have the potential to transition to long term extension. The dose and dosing frequency in the open-label extension for participants from Part 1 will be determined after approval from the regulatory authorities. Additional instructions will be provided in the event there is a treatment gap longer than 6 weeks for patients participating in Part 1.

### Following Part A or B

Participants from Parts A and B, will have the potential to enroll in Part C. Part C is an open-label extension of the trial that will assess the safety and durability of effect on seizures and other outcomes measures of a maintenance dosing regimen over up to 2 years (see Table 4 for the schedule of activities in this part).

The highest dose administered in Part C will be up to the maximum tolerated single dose from Part A and Part B. This dose level may change as Part A and Part B progress. It is expected that dosing every 12 weeks will be sufficient to maintain KD levels. Therefore, dosing will be at least 12 weeks apart for up to 2 years. Participants may receive lower doses or less frequent dosing based on their tolerability.

A titration dose regimen may be added for participants who received only placebo procedures in Part A or Part B. The starting dose for this titration regimen will be no more than the maximum tolerated single dose from Part A or Part B.

The follow-up period may be extended based on the results of the assessments at the end of study (EOS) visit and the investigator's best clinical judgement. Should it be considered necessary, the extended follow-up will include assessments for the presence of potential long-term and developmental effects.

TABLE 1
Schedule of Activities - Part 1

| Trial Period | | | | | | | |
|---|---|---|---|---|---|---|---|
| Screening | | Open-Label Intervention | | | Follow-up | | |
| Visit ^{a} | | | | | | | |
| | Baseline | Baseline | | Dosing | Interim | Interim | |
| | Observation | Dosing | Home | Visit | Follow-up | Follow-up | |
| Screening | Period | Visit | Visit ^{b} | 2-4/EOT ^{c} | Visit 1 ^{d e} | Visit 2 ^{d e} | EOS ^{d} |
| Visit Day | | | | | | | |
| | | | 2 weeks | 4 weeks | | | |
| | | | after | after | 6 weeks | 12 weeks | 24 weeks |
| | | | dosing | previous | after last | after last | after last |
| −56 to −29 | −28 to −1 | 1 | visit ^{f} | dosing ^{f} | dosing | dosing | dosing |

Visit Window (days)

| | | | | |
|---|---|---|---|---|
| +10 | +7 | +10 | ±7 | ±7 |

STUDY ENTRY AND GENERAL ASSESSMENTS

| | | | | |
|---|---|---|---|---|
| Informed consent ^{g} | X | | | |
| Inclusion/exclusion | X | X ^{h} | | |
| criteria | | | | |
| Confirm variant | X | | | |
| characterization ⁱ | | | | |
| Medical and | X | | | |
| disease history | | | | |
| Demographic data | X | | | |
| MRI-brain ^{j} | X | | | |
| Body weight/height | X | X | X | X |
| Pregnancy test ^{k} | X | X | X | X |
| | (serum) | | | |
| ERC review | X | | | |
| and approval | | | | |
| Inpatient stay/ | | X ^{l} | X ^{l} | X |
| clinic visits | | | | |

SAFETY ASSESSMENTS

| | | | | | | | |
|---|---|---|---|---|---|---|---|
| Physical examination | X | X | X | X | X | X | X |
| including neurological | | | | | | | |
| examination ^{m} | | | | | | | |
| Clinical laboratory | X | X | X | X | X | X | X |
| evaluations ⁿ | | | | | | | |
| Levels of concomitant | X | | X | | X | X | |
| ASMs (plasma) ^{o} | | | | | | | |
| CSF collection for | | X | | X | | | |
| protein, glucose, | | | | | | | |
| cell count, and | | | | | | | |
| differential ^{p} | | | | | | | |
| Vital signs ^{q} | X | X | X | X | X | X | X |
| 12-lead ECG ^{r} | X | X | X | X | X | X | X |

| | |
|---|---|
| AE monitoring and | X |
| recording ^{s} | |
| Concomitant meds/ | X |
| procedures ^{s} | |

EFFICACY ASSESSMENTS

| Seizure diary ^{t} | X | | | |
|---|---|---|---|---|
| vEEG ^{u} | | X | X | X |

PHARMACOKINETICS/PHARMACODYNAMICS

| | | | | | | |
|---|---|---|---|---|---|---|
| Study drug | | X | X | X | X | X |
| concentration | | | | (plasma | (plasma | (plasma |
| (plasma and/or CSF) ^{v} | | | | only) | only) | only) |
| Inflammatory markers | | X | X | | | X |
| (serum) ^{w} | | | | | | |
| Biomarkers potentially | X | X | X | | | X |
| associated with SCN2A | (plasma | (CSF | | | | (plasma |
| GoF incl. mRNA, Naᵥ1.2 | only) | only) | | | | only) |
| protein/peptides | | | | | | |
| (plasma, CSF) ^{x} | | | | | | |
| ADA levels (serum) ^{y} | | X | X | X | X | X |

STUDY DRUG

| | | |
|---|---|---|
| Compound 1 IT | X ^{v} | X |
| administration ^{z} | | |

ADA = anti-drug antibodies; AE = adverse event; ECG = electrocardiogram; EEG = electroencephalogram; ERC = Eligibility Review Committee; EOS = end of study; EOT = end of treatment; HCG = human chorionic gonadotropin; IT = intrathecal; meds = medications; MRI = magnetic resonance imaging; SAE = serious adverse event; vEEG = video electroencephalogram.
^{a} Each visit that spans multiple days allows, amongst others, for complex behavioral assessments to be carried out over more than 1 day, if required.
^{b} Home visits after dosing may be conducted in Clinic at the discretion of the investigator/caregiver.
^{c} Dosing Visit 4 is considered EOT visit. If a subject has early termination, then the last visit at which they were dosed would be considered the EOT.
^{d} Follow-up visits (Interim follow up visit 1 and 2, and EOS) are required for participants who complete dosing and do not rollover into open-label extension or who discontinue treatment early. In the event that the open label extension is enrolling, participants will not complete interim follow up visit 1 and 2, and EOS. In the event that the open label extension is not enrolling after the completion of 4 doses, participants will continue to be followed at interim follow-up visits 1, 2, and at EOS. Following the completion of the EOS visit, if Part C/open label extension is enrolling patients may re-enroll in Part C/open label extension.
^{e} Interim follow-up visits 1 and 2 can be Home or Clinic visits.
^{f} Dosing visits will occur at ≥4-week intervals. Home Visits in-between dosing visits will take place 2 weeks after each Dosing Visit.
^{g} Parent/guardian/caregiver is required to sign an informed consent form prior to participant entering Screening (and participant to provide assent, if applicable).
^{h} To be performed prior to randomization to confirm eligibility.
ⁱ Variant characterization performed prior to the screening window may be used for trial entry. Consent for the variant characterization must be obtained if it is to be performed specifically for the purpose of this trial.
^{j} Brain MRI only to be performed during Screening if the patient does not have images available for review from a brain MRI performed within 6 months of Screening. If a brain MRI has not been performed within 6 months of screening, the participant will need to have MRI brain without gadolinium as part of Screening to assess ventricle size. Alternatively, in patients who have had a historical MRI, a brain CT scan without contrast may be performed at the discretion of the investigator to assess ventricle size.
^{k} hCG (serum) is required for females of childbearing potential at Screening- Post-screening testing for females of childbearing potential may be performed via serum or urine.
Note:
Pregnancy testing may be waived at the discretion of the investigator if it is established that the participant is not sexually active/at risk for pregnancy.
^{l} Participants are required to be inpatient for a minimum of 24 hours, and up to 48 hours, after each dose administration, but sites can elect to keep participants inpatient for a longer duration in accordance with their institutional procedures.
Note.
such elected prolongation of inpatient stay, not associated with untoward medical occurrence, would not meet the definition of an AE or SAE.
^{m} Neurological examination specifically targeting mental status, cranial nerves (including, but not limited to, eye movements, in particular nystagmus, jaw and facial movements, and swallowing/drooling), motor function (including, but not limited to, upper and lower strength and symmetry), appendicular and axial tone, sensory (including, but not limited to, withdraw to various stimuli), reflexes (including, but not limited to, decreased or increased, evidence of clonus, absent), coordination (including upper and lower limbs, symmetry, or evidence of tremor or involuntary movements), and gait, if applicable. Explicit documentation requirement of neurological examination, including a detailed checklist/worksheet for clinicians performing the examinations will be provided.
ⁿ Clinical laboratories at Screening (central laboratory only) will include hematology, coagulation, clinical chemistry, hepatitis and HIV screen, and urinalysis. Clinical laboratories at Dosing Visits will include coagulation parameters and platelet count collected between 24 hours and 36 hours prior to dosing (local laboratory only), and the results will need to be reviewed by the investigator or designee within 24 hours prior to dosing. Hematology, clinical chemistry and urinalysis (central laboratory only) will also be collected. Clinical laboratories at IIome Visits after each dosing visit (central laboratory only) will include coagulation, clinical chemistry, hematology, and urinalysis. Clinical laboratories at Interim follow up visit 1 and 2, and EOS (central laboratory only) will include hematology, coagulation, clinical chemistry, and urinalysis. For participants for whom blood volume is a concern, consult the sponsor medical monitor for prioritization of blood samples.
^{o} For subjects for whom blood volume is a concern, consult the sponsor medical monitor for prioritization of blood samples.
^{p} CSF will be collected at the time of each study drug administration, for protein, glucose, cell count, and differential according to local laboratory requirements. Further details included in study laboratory and/or procedure manual. CSF biomarkers will be processed centrally. For children in whom CSF sampling volume is a concern, the following will be prioritized: CSF protein, PK and biomarkers.
^{q} Vital signs include temperature, respiratory rate, supine blood pressure (systolic and diastolic), and heart rate. Participants should rest for approximately 5 minutes prior to blood pressure and heart rate measurements.
^{r} Triplicate ECG measurement will be taken at Screening and pre-dose at Baseline Dosing Visit; for all other timepoints, single measurement will be taken.
^{s} Will be collected throughout the clinical trial from the time of signing informed consent until the end of the clinical trial.
^{t} Caregivers will complete a daily seizure diary for at minimum 4 weeks during the Baseline Observation period (prior to Baseline Dosing Visit) through EOT, including the days without visits. Other screening assessments may still occur during this 4-week period (and do not necessarily have to be completed prior to the start of the daily seizure diary).
^{u} Prolonged video EEG (vEEG) can be completed at home or in a facility, ideally with a minimum of 12 hours of continuous recording capturing both wakefulness and sleep. Baseline vEEG should be completed within the 28 days prior to Baseline Dosing Visit. Some participants may not tolerate the vEEG for a full 12 hours and this will not result in a protocol deviation; however, the reason(s) for the inability to obtain/complete the vEEG for a full 12 or more hours should be documented.
^{v} Plasma PK samples will be collected at Baseline Dosing Visit and at 4^{th} dosing visit at 1, 2, 4, 8, and 24 hours post-dose (within 15 minutes).
(Note:
Sponsor may adjust PK sampling schedule to align with recommended blood volume restrictions).
Plasma and CSF samples will be collected at every dosing visit predose (at the time of lumbar puncture for study drug administration). At interim follow up visit 1,, interim follow-up visit 2, and EOS visit - collect a plasma PK sample only at any time during visit. Further details included in study laboratory and/or procedure manual. For children in whom maximum blood sampling volume is a concern consult the Sponsor's medical monitor. For children in whom CSF sampling volume is a concern, the following will be prioritized: CSF protein, PK and biomarkers.
^{w} CRP, CH50/C3a/Bb/C5a, and fibrinogen to be collected predose (any time on the day of dosing prior to dose administration), and at EOS. For subjects for whom blood volume is a concern, consult the sponsor medical monitor for prioritization of blood samples.
^{x} The CSF and plasma samples for biomarkers potentially associated with SCN2A GoF are to be stored only but may be analyzed at a later date. Plasma samples for biomarkers will be collected 24-hour post-dose at Dosing Visit 3 only. For subjects for whom blood volume is a concern, consult the sponsor medical monitor for prioritization of blood samples. For children in whom CSF sampling volume is a concern, the following will be prioritized: CSF protein, PK and biomarkers.
^{y} At every Dosing Visit, samples for ADA levels will be collected between 24-hour and 36-hour prior to dosing For subjects for whom blood volume is a concern, consult the sponsor medical monitor for prioritization of blood samples.
^{z} First dosing will only take place after continued eligibility has been confirmed. Participants will receive doses of Compound 1 via IT administration at each dosing visit. Further details included in study procedure manual.

TABLE 2
Schedule of Activities - Part A

| Trial Period | | | | | | | |
|---|---|---|---|---|---|---|---|
| Screening | | Double-Blind Intervention | | | Follow-up | | |
| Visit ^{a} | | | | | | | |
| | Baseline | Baseline | | Dosing | Interim | Interim | |
| | Observation | Dosing | Home | Visit | Follow-up | Follow-up | |
| Screening | Period | Visit | Visit ^{b} | 2-6/ EOT ^{c} | Visit 1 ^{d} | Visit 2 ^{d} | EOS ^{d} |
| Visit Day | | | | | | | |
| | | | | 4 weeks | | | |
| | | | 2 weeks | or 6 weeks | | | |
| | | | after | after | 6 weeks | 12 weeks | 24 weeks |
| | | | dosing | previous | after last | after last | after last |
| −56 to −29 | −28 to −1 | 1 | visit ^{e} | dosing ^{e} | dosing | dosing | dosing |

Visit Window (days)

| | | | | |
|---|---|---|---|---|
| +10 | +7 | +10 | ±7 | ±7 |

STUDY ENTRY AND GENERAL ASSESSMENTS

| | | | | |
|---|---|---|---|---|
| Informed consent ^{f} | X | | | |
| Inclusion/exclusion | X | X ^{g} | | |
| criteria | | | | |
| Confirm variant | X | | | |
| characterization ^{h} | | | | |
| Medical and | X | | | |
| disease history | | | | |
| Demographic data | X | | | |
| MRI-brain ⁱ | X | | | |
| Body weight/height | X | X | X | X |
| Pregnancy test ^{j} | X | X | X | X |
| | (serum) | | | |
| ERC review and | X | | | |
| approval | | | | |
| Inpatient stay/ | | X ^{k} | X ^{k} | X |
| clinic visits | | | | |

SAFETY ASSESSMENTS

| | | | | | | | |
|---|---|---|---|---|---|---|---|
| Physical examination | X | X | X | X | X | X | X |
| including neurological | | | | | | | |
| examination ^{l} | | | | | | | |
| Clinical laboratory | X | X | X | X | X | X | X |
| evaluations ^{m} | | | | | | | |
| Levels of concomitant | X | | X | X ⁿ | X | X | |
| ASMs (plasma) | | | | | | | |
| CSF collection for | | X | | X | | | |
| protein, glucose, | | | | | | | |
| cell count, and | | | | | | | |
| differential ^{o} | | | | | | | |
| Vital signs ^{p} | X | X | X | X | X | X | X |
| 12-lead ECG ^{q} | X | X | X | X | X | X | X |

| | |
|---|---|
| AE monitoring | X |
| and recording ^{r} | |
| Concomitant meds/ | X |
| procedures ^{r} | |

EFFICACY ASSESSMENTS

| Seizure diary ^{s} | X | | | | | | | |
|---|---|---|---|---|---|---|---|---|
| vEEG ^{t} | | X | | X | | X | | |
| Bayley-4 or WPPSI- | | | X ^{u} | | X ^{v} | | | X |
| IV (video recorded) | | | | | | | | |
| Vineland-3 | | X | X | | X | X | X | X |
| Sleep Disturbance | | | X | | X | X | X | X |
| Scale for Children | | | | | | | | |
| Quality of Life | | | X | | X | X | X | X |
| Inventory-Disability | | | | | | | | |
| (QI-Disability) | | | | | | | | |
| ABC-2 | | | X | | X | X | X | X |
| CGI-S and CgGI-S | | | X | | | | | |
| CGI-I and CgGI-I | | | | | X | | | X |

PHARMACOKINETICS/PHARMACODYNAMICS

| | | | | | | |
|---|---|---|---|---|---|---|
| Study drug concentration | | X | X | X | X | X |
| (plasma and/or CSF) ^{w} | | | | (plasma | (plasma | (plasma |
| | | | | only) | only) | only) |
| Inflammatory markers | | X | X | | | X |
| (serum) ^{x} | | | | | | |
| Biomarkers potentially | X | X | X | | | X |
| associated with SCN2A | (plasma | (CSF | | | | (plasma |
| GoF incl. mRNA, Naᵥ1.2 | only) | only) | | | | only) |
| protein/peptides | | | | | | |
| (plasma, CSF) ^{y} | | | | | | |
| ADA levels (serum) ^{z} | | X | X | X | X | X |

STUDY DRUG

| | | |
|---|---|---|
| Randomization | X ^{aa} | |
| Compound 1 IT | X | X |
| administration, OR | | |
| placebo procedure ^{bb} | | |

ABC-2 = Aberrant Behavior Checklist-2^{nd} Edition; ADA = anti-drug antibodies; AE = adverse event; Bayley-4 = Bayley Scale of Infant Development-4; CGI-I = Clinical Global Impression-Improvement; CGI-S = Clinical Global Impression-Severity; CgGI-I = Caregiver Global Impression-Improvement; CgGI-S = Caregiver Global Impression-Severity; ECG = electrocardiogram; EEG = electroencephalogram; ERC = Eligibility Review Committee; EOS = end of study; EOT = end of treatment (including early termination); HCG = human chorionic gonadotropin; IT = intrathecal; meds = medications; MRI = magnetic resonance imaging; SAE = serious adverse event; Vineland-3 = Vineland Adaptive Behavior Scale-3; vEEG = video electroencephalogram; WPPSI-IV = Wechsler Preschool and Primary Scales of Intelligence, Fourth Edition.
^{a} Each visit that spans multiple days allows, amongst others, for complex behavioral assessments (including but not limited to Bayley-4, WPPSI-IV, etc.) to be carried out over more than 1 day, if required.
^{b} Home visits after dosing may be conducted in Clinic at the discretion of the investigator/caregiver.
^{c} Dosing Visit 6 is considered EOT visit. If a participant has early termination, then the last visit at which they were dosed would be considered the EOT.
^{d} Follow-up visits (Interim follow up visit 1 and 2, and EOS) are not required for participants who will continue in Part C. These visits are required for participants who complete dosing and do not rollover into Part C or who discontinue treatment early. Interim follow-up visits 1 and 2 can be Home or Clinic visits.
^{e} Dosing visits will occur at ≥4-week intervals for the first 4 doses, and at ≥6-week intervals for the subsequent doses. Home Visits in-between dosing visits will take place 2 weeks after each Dosing Visit.
^{f} Parent/guardian/caregiver is required to sign an informed consent form prior to participant entering Screening (and participant to provide assent, if applicable).
^{g} To be performed prior to randomization to confirm eligibility.
^{h} Variant characterization performed prior to the screening window may be used for trial entry. Consent for the variant characterization must be obtained if it is to be performed specifically for the purpose of this trial.
ⁱ Brain MRI only to be performed during Screening if the patient does not have images available for review from a brain MRI performed within 6 months of Screening. If a brain MRI has not been performed within 6 months of screening, the participant will need to have MRI brain without gadolinium as part of Screening to assess ventricle size. Alternatively, in patients who have had a historical MRI, a brain CT scan without contrast may be performed at the discretion of the investigator to assess ventricle size.
^{j} hCG (serum) is required for females of childbearing potential at Screening- Post-screening testing for females of childbearing potential may be performed via serum or urine.
Note:
Pregnancy testing may be waived at the discretion of the investigator if it is established that the participant is not sexually active/at risk for pregnancy.
^{k} Participants are required to be inpatient for a minimum of 24 hours, and up to 48 hours, after each dose administration, but sites can elect to keep participants inpatient for a longer duration in accordance with their institutional procedures.
Note.
Such elected prolongation of inpatient stay, not associated with untoward medical occurrence, would not meet the definition of an AE or SAE.
^{l} Neurological examination specifically targeting mental status, cranial nerves (including, but not limited to, eye movements, in particular nystagmus, jaw and facial movements, and swallowing/drooling), motor function (including, but not limited to, upper and lower strength and symmetry), appendicular and axial tone, sensory (including, but not limited to, withdraw to various stimuli), reflexes (including, but not limited to, decreased or increased, evidence of clonus, absent), coordination (including upper and lower limbs, symmetry, or evidence of tremor or involuntary movements), and gait, if applicable. Explicit documentation requirement of neurological examination, including a detailed checklist/worksheet for clinicians performing the examinations will be provided.
^{m} Clinical laboratories at Screening (central laboratory only) will include hematology, coagulation, clinical chemistry, hepatitis and HIV screen, and urinalysis. Clinical laboratories at Dosing Visits will include coagulation parameters and platelet count collected between 24 hours and 36 hours prior to dosing (local laboratory only), and the results will need to be reviewed by the investigator or designee within 24 hours prior to dosing. Hematology, clinical chemistry and urinalysis (central laboratory only) will also be collected. Clinical laboratories at Home Visits after each dosing visit (central laboratory only) will include coagulation, clinical chemistry, hematology, and urinalysis. Clinical laboratories at Interim follow up visits 1 and 2, and EOS (central laboratory only) will include hematology, coagulation, clinical chemistry, and urinalysis. For participants for whom blood volume is a concern, consult the sponsor medical monitor for prioritization of blood samples.
ⁿ Plasma for concomitant ASM levels will not be collected at Dosing Visit 6.
^{o} CSF will be collected at the time of each study drug administration, for those participants randomized to study drug and not placebo procedure, for protein, glucose, cell count, and differential according to local laboratory requirements. Further details included in study laboratory and/or procedure manual. CSF biomarkers will be processed centrally. For children in whom CSF sampling volume is a concern, the following will be prioritized: CSF protein, PK and biomarkers
^{p} Vital signs include temperature, respiratory rate, supine blood pressure (systolic and diastolic), and heart rate. Participants should rest for approximately 5 minutes prior to blood pressure and heart rate measurements.
^{q} Triplicate ECG measurement will be taken at Screening and predose at Baseline Dosing Visit; for all other timepoints, single measurement will be taken
^{r} Will be collected throughout the clinical trial from the time of signing informed consent until the end of the clinical trial.
^{s} Caregivers will complete a daily seizure diary for at minimum 4 weeks during the Baseline Observation period (prior to Baseline Dosing Visit) through EOT, including the days without visits. Other screening assessments may still occur during this 4-week period (and do not necessarily have to be completed prior to the start of the daily seizure diary).
^{t} Prolonged video EEG (vEEG) can be completed at home or in a facility, ideally with a minimum of 12 hours of continuous recording capturing both wakefulness and sleep. Baseline vEEG should be completed within the 28 days prior to Baseline Dosing Visit. Some participants may not tolerate the vEEG for a full 12 hours and this will not result in a protocol deviation; however, the reason(s) for the inability to obtain/complete the vEEG for a full 12 or more hours should be documented.
^{u} Bayley-4 or WPPSI-IV at Baseline Dosing Visit must be done prior to actual dosing.
^{v} Between Baseline Dosing Visit and EOS, Bayley-4 or WPPSI-IV will only be performed at 4 weeks after Dosing Visit #6 (±1 week), in clinic.
^{w} Plasma PK samples will be collected at Baseline Dosing Visit and at 6^{th} dosing visit at 1, 2, 4, 8, and 24 hours post-dose (within 15 minutes).
(Note:
Sponsor may adjust PK sampling schedule to align with recommended blood volume restrictions).
Plasma will be collected at every dosing visit predose. CSF samples will be collected at every dosing visit predose at the time of lumbar puncture for study drug administration for those participants randomized to study drug and not placebo procedure. At Interim follow-up visits 1 and 2, and EOS visit - collect a plasma PK sample only at anytime during visit. Further details included in study laboratory and/or procedure manual. For children who are lower weight where maximum blood sampling volume or CSF sampling volume is a concern, consult the Sponsor's medical monitor. For children in whom CSF sampling volume is a concern, the following will be prioritized: CSF protein, PK and biomarkers.
^{x} CRP, CH50/C3a/Bb/C5a, and fibrinogen to be collected predose (any time on the day of dosing prior to dose administration), and at EOS. For participants for whom blood volume is a concern, consult the sponsor medical monitor for prioritization of blood samples.
^{y} The CSF and plasma samples for biomarkers potentially associated with SCN2A GoF are to be stored only but may be analyzed at a later date. Plasma for biomarkers will be collected 24-hour post-dose at Dosing Visit 2, 3, 4, 5, and 6. For participants for whom blood volume is a concern, consult the sponsor medical monitor for prioritization of blood samples. For children in whom CSF sampling volume is a concern, the following will be prioritized: CSF protein, PK and biomarkers.
^{z} Samples for ADA levels collected predose (anytime between 24-hours and 36-hours prior to dosing) on dosing visit days, and anytime at Interim follow-up visits 1 and 2, and EOS visit. For participants for whom blood volume is a concern, consult the sponsor medical monitor for prioritization of blood samples.
^{aa} Randomization will be completed prior to first dosing only after continued eligibility has been confirmed.
^{bb} Participants will receive doses of Compound 1 via IT administration or undergo placebo procedure per the randomization schedule on each dosing visit. Further details included in study procedure manual.

TABLE 3
Schedule of Activities - Part B

| Trial Period | | | | | | | |
|---|---|---|---|---|---|---|---|
| Screening | | Double-Blind Intervention | | | Follow-up | | |
| Visit ^{a} | | | | | | | |
| | Baseline | Baseline | | Dosing | Interim | Interim | |
| | Observation | Dosing | Home | Visit | Follow-up | Follow-up | |
| Screening | Period | Visit | Visit ^{b} | 2-6/EOT ^{c} | Visit 1 ^{d} | Visit 2 ^{d} | EOS ^{d} |
| Visit Day | | | | | | | |
| | | | 2 weeks | ≥6 weeks | | | |
| | | | after | after | 6 weeks | 12 weeks | 24 weeks |
| | | | dosing | previous | after last | after last | after last |
| −56 to −29 | −28 to −1 | 1 | visit ^{e} | dosing | dosing | dosing | dosing |

Visit Window (days)

| | | | | |
|---|---|---|---|---|
| +10 | +7 | +10 | ±7 | ±7 |

STUDY ENTRY AND GENERAL ASSESSMENTS

| | | | | |
|---|---|---|---|---|
| Informed consent ^{f} | X | | | |
| Inclusion/exclusion | X | X ^{g} | | |
| criteria | | | | |
| Confirm variant | X | | | |
| characterization ^{h} | | | | |
| Medical and | X | | | |
| disease history | | | | |
| Demographic data | X | | | |
| MRI-brain ⁱ | X | | | |
| Body weight/height | X | X | X | X |
| Pregnancy test ^{j} | X | X | X | X |
| | (serum) | | | |
| ERC review | X | | | |
| and approval | | | | |
| Inpatient stay/ | | X ^{k} | X ^{k} | X |
| clinic visits | | | | |

SAFETY ASSESSMENTS

| | | | | | | | |
|---|---|---|---|---|---|---|---|
| Physical examination | X | X | X | X | X | X | X |
| including neurological | | | | | | | |
| examination ^{l} | | | | | | | |
| Clinical laboratory | X | X | X | X | X | X | X |
| evaluations ^{m} | | | | | | | |
| Levels of concomitant | X | | X | X ⁿ | X | X | |
| ASMs (plasma) | | | | | | | |
| CSF collection for | | X | | X | | | |
| protein, glucose, | | | | | | | |
| cell count, and | | | | | | | |
| differential ^{o} | | | | | | | |
| Vital signs ^{p} | X | X | X | X | X | X | X |
| 12-lead ECG ^{q} | X | X | X | X | X | X | X |

| | |
|---|---|
| AE monitoring | X |
| and recording ^{r} | |
| Concomitant meds/ | X |
| procedures ^{r} | |

EFFICACY ASSESSMENTS

| Seizure diary ^{s} | X | | | | | | | |
|---|---|---|---|---|---|---|---|---|
| vEEG ^{t} | | X | | X | | X | | |
| Bayley-4 or WPPSI- | | | X ^{u} | | X ^{v} | | | X |
| IV (video recorded) | | | | | | | | |
| Vineland-3 | | X | X | | X | X | X | X |
| Sleep Disturbance | | | X | | X | X | X | X |
| Scale for Children | | | | | | | | |
| Quality of Life | | | X | | X | X | X | X |
| Inventory-Disability | | | | | | | | |
| (QI-Disability) | | | | | | | | |
| ABC-2 | | | X | | X | X | X | X |
| CGI-S and CgGI-S | | | X | | | | | |
| CGI-I and CgGI-I | | | | | X | | | X |

PHARMACOKINETICS/PHARMACODYNAMICS

| | | | | | | |
|---|---|---|---|---|---|---|
| Study drug concentration | | X | X | X | X | X |
| (plasma and/or CSF) ^{w} | | | | (plasma | (plasma | (plasma |
| | | | | only) | only) | only) |
| Inflammatory markers | | X | X | | | X |
| (serum) ^{x} | | | | | | |
| Biomarkers potentially | X | X | X | | | X |
| associated with SCN2A | (plasma | (CSF | | | | (plasma |
| GoF incl. mRNA, Naᵥ1.2 | only) | only) | | | | only) |
| protein/peptides | | | | | | |
| (plasma, CSF) ^{y} | | | | | | |
| ADA levels (serum) ^{z} | | X | X | X | X | X |

STUDY DRUG

| | | |
|---|---|---|
| Randomization | X ^{aa} | |
| Compound 1 IT administration, | X | X |
| OR placebo procedure ^{bb} | | |

ABC-2 = Aberrant Behavior Checklist-2^{nd} Edition; ADA = anti-drug antibodies; AE = adverse event; Bayley-4 = Bayley Scale of Infant Development-4; CGI-I = Clinical Global Impression-Improvement; CGI-S = Clinical Global Impression-Severity; CgGI-I = Caregiver Global Impression-Improvement; CgGI-S = Caregiver Global Impression-Severity; ECG = electrocardiogram; EEG = electroencephalogram; ERC = Eligibility Review Committee; EOS = end of study; EOT (including early termination) = end of treatment; HCG = human chorionic gonadotropin; IT = intrathecal; meds = medications; MRI = magnetic resonance imaging; SAE = serious adverse event; Vineland-3 = Vineland Adaptive Behavior Scale-3; vEEG = video electroencephalogram; WPPSI-IV = Wechsler Preschool and Primary Scales of Intelligence, Fourth Edition.
^{a} Each visit that spans multiple days allows, amongst others, for complex behavioral assessments (including but not limited to Bayley-4, WPPSI-IV, etc.) to be carried out over more than 1 day, if required.
^{b} Home visits after dosing may be conducted in Clinic at the discretion of the investigator/caregiver.
^{c} Dosing Visit 6 is considered EOT visit. If a subject has early termination, then the last visit at which they were dosed would be considered the EOT.
^{d} Follow-up visits (Interim follow up visit 1 and 2, and EOS) are not required for participants who will continue in Part C. Follow-up visits (Interim and EOS) are required for participants who complete dosing and do not rollover into Part C or who discontinue treatment early. Interim follow-up visits 1 and 2 can be Home or Clinic visits.
^{e} Home Visits in-between Dosing Visits 2-6 will take place 2 weeks after each Dosing Visit.
^{f} Parent/guardian/caregiver is required to sign an informed consent form prior to participant entering Screening (and participant to provide assent, if applicable).
^{g} To be performed prior to randomization to confirm eligibility.
^{h} Variant characterization performed prior to the screening window may be used for trial entry. Consent for the variant characterization must be obtained if it is to be performed specifically for the purpose of this trial.
ⁱ Brain MRI only to be performed during Screening if the patient does not have report available for review from a brain MRI performed within 6 months of Screening. If a brain MRI has not been performed within 6 months of screening, the participant will need to have MRI brain without gadolinium as part of Screening to assess ventricle size. Alternatively, in patients who have had a historical MRI, a brain CT scan without contrast may be performed at the discretion of the investigator to assess ventricle size.
^{j} hCG (serum) is required for females of childbearing potential at Screening- Post-screening testing for females of childbearing potential may be performed via serum or urine.
Note:
Pregnancy testing may be waived at the discretion of the investigator if it is established that the participant is not sexually active/at risk for pregnancy.
^{k} Participants are required to be inpatient for a minimum of 24 hours, and up to 48 hours, after each dose administration, but sites can elect to keep participants inpatient for a longer duration in accordance with their institutional procedures.
Note.
Such elected prolongation of inpatient stay, not associated with untoward medical occurrence, would not meet the definition of an AE or SAE.
^{l} Neurological examination specifically targeting mental status, cranial nerves (including, but not limited to, eye movements, in particular nystagmus, jaw and facial movements, and swallowing/drooling), motor function (including, but not limited to, upper and lower strength and symmetry), appendicular and axial tone, sensory (including, but not limited to, withdraw to various stimuli), reflexes (including, but not limited to, decreased or increased, evidence of clonus, absent), coordination (including upper and lower limbs, symmetry, or evidence of tremor or involuntary movements), and gait, if applicable. Explicit documentation requirement of neurological examination, including a detailed checklist/worksheet for clinicians performing the examinations will be provided.
^{m} Clinical laboratories at Screening (central laboratory only) will include hematology, coagulation, clinical chemistry, hepatitis and HIV screen, and urinalysis. Clinical laboratories at Dosing Visits will include coagulation parameters and platelet count collected between 24 hours and 36 hours prior to dosing (local laboratory only), and the results will need to be reviewed by the investigator or designee within 24 hours prior to dosing. Hematology, clinical chemistry and urinalysis (central laboratory only) will also be collected. Clinical laboratories at Home Visits after each dosing visit (central laboratory only) will include coagulation, clinical chemistry, hematology, and urinalysis. Clinical laboratories at Interim follow up visits 1 and 2, and EOS (central laboratory only) will include hematology, coagulation, clinical chemistry, and urinalysis. For participants for whom blood volume is a concern, consult the sponsor medical monitor for prioritization of blood samples.
ⁿ Plasma for concomitant ASM levels will not be collected at Dosing Visit 6.
^{o} CSF will be collected at the time of each study drug administration, for those participants randomized to study drug and not placebo procedure, for protein, glucose, cell count, and differential according to local laboratory requirements. Further details included in study laboratory and/or procedure manual. CSF biomarkers will be processed centrally. For children in whom CSF sampling volume is a concern, the following will be prioritized: CSF protein, PK and biomarkers
^{p} Vital signs include temperature, respiratory rate, supine blood pressure (systolic and diastolic), and heart rate. Participants should rest for approximately 5 minutes prior to blood pressure and heart rate measurements.
^{q} Triplicate ECG measurement will be taken at Screening and predose at Baseline Dosing Visit; for all other timepoints, single measurement will be taken
^{r} Will be collected throughout the clinical trial from the time of signing informed consent until the end of the clinical trial.
^{s} Caregivers will complete a daily seizure diary for at minimum 4 weeks during the Baseline Observation period (prior to Baseline Dosing Visit) through EOT, including the days without visits. Other screening assessments may still occur during this 4-week period (and do not necessarily have to be completed prior to the start of the daily seizure diary).
^{t} Prolonged video EEG (vEEG) can be completed at home or in a facility, ideally with a minimum of 12 hours of continuous recording capturing both wakefulness and sleep. Baseline vEEG should be completed within the 28 days prior to Baseline Dosing Visit. Some participants may not tolerate the vEEG for a full 12 hours and this will not result in a protocol deviation; however, the reason(s) for the inability to obtain/complete the vEEG for a full 12 or more hours should be documented.
^{u} Bayley-4 or WPPSI-IV at Baseline Dosing Visit must be done prior to actual dosing.
^{v} Between Baseline Dosing Visit and EOS, Bayley-4 or WPPSI-IV will only be performed at 4 weeks after Dosing Visit #6 (±1 week), in clinic.
^{w} Plasma PK samples will be collected at Baseline Dosing Visit and at 6^{th} dosing visit at 1-, 2-, 4-, 8-, and 24-hours post-dose (within 15 minutes).
(Note:
Sponsor may adjust PK sampling schedule to align with recommended blood volume restrictions).
Plasma will be collected at every dosing visit predose. CSF samples will be collected at every dosing visit predose at the time of lumbar puncture for study drug administration for those participants randomized to study drug and not placebo procedure. At Interim follow-up visits 1 and 2, and EOS visit- collect a plasma PK sample only at anytime during visit. Further details included in study laboratory and/or procedure manual. For children who are lower weight where maximum blood sampling volume or CSF sampling volume is a concern, consult the Sponsor's medical monitor. For children in whom CSF sampling volume is a concern, the following will be prioritized: CSF protein, PK and biomarkers.
^{x} CRP, CH50/C3a/Bb/C5a, and fibrinogen to be collected predose (any time on the day of dosing prior to dose administration), and at EOS. For participants for whom blood volume is a concern, consult the sponsor medical monitor for prioritization of blood samples.
^{y} The CSF and plasma samples for biomarkers potentially associated with SCN2A GoF are to be stored only but may be analyzed at a later date. Plasma samples for biomarkers will be collected 24-hour post-dose at Dosing Visit 2, 3, 4, 5, and 6. For participants for whom blood volume is a concern, consult the sponsor medical monitor for prioritization of blood samples. For children in whom CSF sampling volume is a concern, the following will be prioritized: CSF protein, PK and biomarkers.
^{z} Samples for ADA levels collected predose (anytime between 24-hours and 36-hours prior to dosing) on dosing visit days, and any time at Interim follow-up visits 1 and 2, and EOS visit. For participants for whom blood volume is a concern, consult the sponsor medical monitor for prioritization of blood samples.
^{aa} Randomization will be completed prior to first dosing only after continued eligibility has been confirmed.
^{bb} Participants will receive doses of Compound 1 via IT administration or undergo placebo procedure per the randomization schedule on each dosing visit.

TABLE 4
Schedule of Activities - Part C (Following Completion of Part A or B)

| Trial Period | | | |
|---|---|---|---|
| Open-Label Extension (OLE) | | Follow-up | |
| Visit ^{a} | | | |
| OLE Dosing | Home | Interim | |
| Visits ^{b} | Visit(s) ^{c} | Follow-up Visit | EOS |
| Visit Day | | | |
| ≥12 weeks after last dosing | | | |
| in Part A or Part B | | | |
| OR | | | |
| ≥12 weeks after previous | 2 weeks after | 12 weeks after | 24 weeks after |
| dosing in Part C | dosing visit | last dosing | last dosing |
| Visit Window (days) | | | |
| +7 | ±5 | ±7 | ±7 |

GENERAL ASSESSMENTS

| | | |
|---|---|---|
| Body weight/height | X | X |
| Pregnancy test ^{d} | X | X |
| Inpatient stay/clinic visits | X ^{e} | X |

SAFETY ASSESSMENTS

| | | | | |
|---|---|---|---|---|
| Physical examination including neurological | X | X | X | X |
| examination ^{f} | | | | |
| Clinical laboratory evaluations ^{g} | X | X | X | X |
| Levels of concomitant ASMs (plasma) | X | X | X | |
| CSF collection for protein, glucose, | X | | | |
| cell count, and differential ^{h} | | | | |
| Vital signs ⁱ | X | X | X | X |
| 12-lead ECG ^{j} | X | X | X | X |

| | |
|---|---|
| AE monitoring and recording ^{k} | X |
| Concomitant meds/procedures ^{k} | X |

EFFICACY ASSESSMENTS

| Seizure diary | X | | | |
|---|---|---|---|---|
| vEEG ^{l} | | X | | |
| Bayley-4 or WPPSI-IV (video recorded) | | | | X |
| Vineland-3 | X | | X | X |
| Sleep Disturbance Scale for Children | X | | X | X |
| Quality of Life Inventory-Disability | X | X | X | X |
| (QI-Disability) | | | | |
| ABC-2 | X | | X | X |
| CGI-I and CgGI-I | X | | | X |

PHARMACOKINETICS/PHARMACODYNAMICS

| | | |
|---|---|---|
| Study drug concentration (plasma | X | X |
| and/or CSF) ^{m} | | (plasma only) |
| Inflammatory markers (serum) ⁿ | X | X |
| Biomarkers potentially associated with SCN2A | X | X |
| GoF incl. mRNA, Naᵥ1.2 protein/peptides | | (plasma only) |
| (plasma, CSF) ^{o} | | |
| ADA levels (serum) ^{p} | X | X |

STUDY DRUG

| | |
|---|---|
| Compound 1 IT administration | X |

ABC-2 = berrant Behavior Checklist-2^{nd} Edition; ADA = anti-drug antibodies; AE = adverse event; Bayley-4 = Bayley Scale of Infant Development-4; CGI-I = Clinical Global Impression-Improvement; CgGI-I = Caregiver Global Impression-Improvement; ECG = electrocardiogram; EEG = electroencephalogram; EOS = end of study; EOT (including early termination) = end of treatment; HCG = human chorionic gonadotropin; IT = intrathecal; meds = medications; OLE = Open-Label Extension; SAE = serious adverse event; Vineland-3 = Vineland Adaptive Behavior Scale-3; vEEG = video electroencephalogram; WPPSI-IV = Wechsler Preschool and Primary Scales of Intelligence, Fourth Edition.
^{a} Each visit that spans multiple days allows, amongst others, for complex behavioral assessments (including but not limited to Bayley-4, WPPSI-IV, etc.) to be carried over more than one day, if required.
^{b} Last dosing visit is considered End of Treatment (EOT) visit.
^{c} Home visits may be conducted in Clinic at the discretion of the investigator/caregiver.
^{d} Pregnancy testing for females of childbearing potential may be performed via serum or urine. Note: pregnancy testing may be waived at the discretion of the investigator if it is established that the participant is not sexually active/at risk for pregnancy.
^{e} Participants are required to be inpatient for a minimum of 24 hours, and up to 48 hours, after each dose administration. Sites can elect to keep participants inpatient for a longer duration in accordance with their institutional procedures. Note. Such elected prolongation of inpatient stay, not associated with untoward medical occurrence, would not meet the definition of an AE or SAE.
^{f} Neurological examination specifically targeting mental status, cranial nerves (including, but not limited to, eye movements, in particular nystagmus, jaw and facial movements, and swallowing/drooling), motor function (including, but not limited to, upper and lower strength and symmetry), appendicular and axial tone, sensory (including, but not limited to, withdraw to various stimuli), reflexes (including, but not limited to, decreased or increased, evidence of clonus, absent), coordination (including upper and lower limbs, symmetry, or evidence of tremor or involuntary movements), and gait, if applicable. Explicit documentation requirement of neurological examination, including a detailed checklist/worksheet for clinicians performing the examinations will be provided.
^{g} Clinical laboratories at Dosing Visits will include coagulation parameters and platelet count collected between 24 hours and 36 hours prior to dosing (local laboratory only), and the results will need to be reviewed by the investigator or designee within 24 hours prior to dosing. Hematology, clinical chemistry and urinalysis (central laboratory only) will also be collected. Clinical laboratories at Home Visits after each dosing visit (central laboratory only) will include coagulation, clinical chemistry, hematology, and urinalysis. Clinical laboratories at Interim follow up visit, and EOS (central laboratory only) will include hematology, coagulation, clinical chemistry, and urinalysis. For participants for whom blood volume is a concern, consult the sponsor medical monitor for prioritization of blood samples.
^{h} CSF will be collected at the time of each study drug administration for protein, glucose, cell count, and differential according to local laboratory requirements. Further details included in study laboratory and/or procedure manual. CSF biomarkers will be processed centrally. For children in whom CSF sampling volume is a concern, the following will be prioritized: CSF protein, PK and biomarkers
ⁱ Vital signs include temperature, respiratory rate, supine blood pressure (systolic and diastolic), and heart rate. Participants should rest for approximately 5 minutes prior to blood pressure and heart rate measurements.
^{j} Single ECG measurement will be taken.
^{k} Will be collected throughout the clinical trial until the end of the clinical trial.
^{l} Prolonged video EEG (vEEG) should be completed ideally with a minimum of 12 hours of continuous recording capturing both wakefulness and sleep, with a window of ±5 days. Some participants may not tolerate the vEEG for a full 12 hours and this will not result in a protocol deviation; however, the reason(s) for the inability to obtain/complete the vEEG for a full 12 or more hours should be documented.
^{m} Plasma PK and CSF samples will be collected at every dosing visit predose (at the time of lumbar puncture for study drug administration). At EOS visit-- collect plasma PK specimen only at anytime during visit. Further details included in study laboratory and/or procedure manual. For participants for whom blood volume is a concern, consult the sponsor medical monitor for prioritization of blood samples. For children in whom CSF sampling volume is a concern, the following will be prioritized: CSF protein, PK and biomarkers.
ⁿ CRP, CH50/C3a/Bb/C5a, and fibrinogen to be collected predose (any time on the day of dosing prior to dose administration), and at EOS. For participants for whom blood volume is a concern, consult the sponsor medical monitor for prioritization of blood samples.
^{o} The CSF and plasma samples for biomarkers potentially associated with SCN2A GoF are to be stored only but may be analyzed at a later date. For participants for whom blood volume is a concern, consult the sponsor medical monitor for prioritization of blood samples. For children in whom CSF sampling volume is a concern, the following will be prioritized: CSF protein, PK and biomarkers.
^{p} ADA samples collected predose (any time between 24-hour and 36-hour prior to dose administration at every dosing visit), and at EOS. For participants for whom blood volume is a concern, consult the sponsor medical monitor for prioritization of blood samples.

### Efficacy Assessments

Planned time points for all efficacy assessments are provided in the SoA (Table 1, Table 2, Table 3, Table 4).

Seizure Diary

An electronic device (e.g., tablet/phone, watch application, and/or other devices) will be used to collect information about participant's seizures and daily concomitant medication use during the clinical trial. The diary will be completed daily for a minimum of 28 days prior to Baseline dosing visit, during the Baseline Observation Period (after the Screening visit) and daily throughout the remainder of the clinical trial. The diary from the Baseline Observation Period will serve as an eligibility check, and to establish a baseline. Prompts will be used to ensure adequate data capture in addition to spontaneous reporting. The diary will be completed by the same caregiver as often as possible. Daily seizure diary data collected as part of an ongoing observation trial that falls within the Screening window per this protocol may be used without having to be repeated.

### Video EEG

To the extent allowed by local regulations, a vEEG will be performed by a trained technician throughout the trial in order to record brainwave activity and to evaluate for changes over time. Prolonged vEEG can be completed at home or in a facility, ideally with a minimum of 12 hours of continuous recording and capturing periods of both wakefulness and sleep, vEEGs collected within the Screening window per this protocol, as part of an ongoing observational trial, would not have to be repeated during the Screening period.

### Bayley Scales of Infant Development-Fourth Edition (Bayley-4)

The Bayley-4 is a standardized neurodevelopmental assessment measure used by clinicians to evaluate key domains in early childhood development for individuals between 16 days and 42 months after birth (Bayley and Aylward 2019). These domains include adaptive behavior, cognition, language, motor function (gross and fine), and social-emotional development. The Bayley-4 assessments will be video recorded to the extent allowed by local regulations.

### Vineland Adaptive Behavior Scale-3 (Vineland-3)

The Vineland-3 is a clinician-assessed measure of adaptive behavior in individuals with intellectual disabilities (Sparrow et at 2016). The Vineland-3 includes assessment of communication, socialization, maladaptive behaviors, motor skills, and daily living skills in children as young as 3 years of age.

### Wechsler Preschool and Primary Scales of Intelligence, Fourth Edition (WPPSI-IV)

The WPPSI-IV is a comprehensive test used to assess cognitive function in children from the age of 2 years 6 months to 7 years 7 months. The WPPSI-IV assesses 5 areas: full scale intelligence quotient (IQ), verbal IQ, performance IQ, processing speed, and global language. Investigators will be trained in the administration of the WPPSI-IV.

### Cognitive Test Selection Guide

During the evaluation period, participants will undergo cognitive tests. The cognitive test used (Bayley-4 or WPPSI-IV) will be dependent not only on the participant's age but also, for participants ≥3 years of age, on the participant's initial Vineland-3 score obtained prior to Baseline, as shown in FIG. 2. Vineland-3 is an individually administered measure of behavior that is used to assess individuals with intellectual, developmental, and other neurologic disabilities. The Vineland-3 will be used to assess the participant's cognitive age based on measures of receptive and expressive communication, rather than chronological age, in participants ≥3 years of age as follows:
Participants <3 years (36 months) of age at Baseline (Visit 1): All participants will be assigned to the Bayley-4 cognitive test; and
Participants ≥3 years (36 months) of age at Baseline (Visit 1): All participants will be assigned to either the Bayley-4 or the WPPSI-IV, based upon their initial Vineland-3, Parent/Caregiver version score, as described in FIG. 2. The scores achieved on the receptive communication and expressive communication subscales will determine their cognitive test assignment. Once the cognitive test is assigned, it will not change for the duration of the trial, with the following exception:
   If it is determined by the investigator that the initial Vineland score was invalid secondary to external factors, including but not limited to illness, social stressors, or other events that may have affected the test results; the Vineland-3 may be repeated, and that score used.

### Sleep Disturbance Scale for Children

The parent-reported Sleep Disturbance Scale for Children (SDSC) is a 27-item scale rated on a 5-point Likert scale and designed to categorize sleep disorders in children (Bruni et al 1996). In addition to an overall score, the instrument provides 5 sub-scores for the following: disorders of initiating and maintaining sleep, sleep breathing disorders, disorders of arousal or sleep-wake transition disorders, disorders of excessive somnolence, and sleep hyperhidrosis.

### Quality of Life Inventory-Disability (QI-Disability)

The QI-Disability is a parent-report measure for children with intellectual disabilities. It is a reliable and valid measure of the quality of life across the spectrum of intellectual disability. (Downs et al 2018). It has the potential to allow for clearer identification of support needs and measures responsiveness to interventions.

### Aberrant Behaviors Checklist-2^{nd} Edition (ABC-2)

The ABC-2 is a clinician-assessed rating scale that measures the severity of a range of problem behaviors commonly observed in individuals with intellectual disabilities (Aman and Singh 2017).

### Clinical Global Impression-Severity (CGI-S) and Clinical Global Impression-Improvement (CGI-I)

The CGI was developed for use in NIH-sponsored clinical trials in individuals with mental health disorders. The CGI provides an overall assessment of improvement over a specified period. The CGI includes two, 7-point Likert rating scales: the CGI-S and the CGI-I scale (Guy 1976). The CGI will be anchored with reference to domains/symptoms experienced by participants with SCN2A-DEE. Participants will be assessed by the clinician at Baseline (Day 1) for the severity of SCN2A-DEE symptoms using the CGI-S. Change from baseline in SCN2A-DEE symptoms will be assessed by the clinician using the CGI-I.

### Caregiver Global Impression-Severity (CgGI-S) and Caregiver Global Impression-Improvement (CgGI-I)

These scales are similar to the CGI-S and CGI-I (Guy 1976). Participants will be assessed by the caregiver at Baseline (Day 1) for the severity of their SCN2A-DEE symptoms using the CgGI-S. Change from baseline in SCN2A-DEE symptoms will be assessed by the caregiver using the CgGI-I.

SEQttENCES

| SEQ | |
|---|---|
| ID | |
| NO | Sequence |
| 1 | aacagacattgggtaccatcgaatgactgtcagaacagaaagctaaggcaaaggagggaggatgctgtggtcatcctt |
| | tcttgtttttttcttctttaatgaggatagagcacatgtgagattttactttctactccagtaaaaattctgaagaattgcattgga |
| | gactgttatattcaacacatacgtggattctgtgttatgatttacatttttctttatttcagcactttcttatgcaaggagctaaac |
| | agtgattaaaggagcaggatgaaaagatggcacagtcagtgctggtaccgccaggacctgacagcttccgcttctttac |
| | cagggaatcccttgctgctattgaacaacgcattgcagaagagaaagctaagagacccaaacaggaacgcaaggatg |
| | aggatgatgaaaatggcccaaagccaaacagtgacttggaagcaggaaaatctcttccatttatttatggagacattcctc |
| | cagagatggtgtcagtgcccctggaggatctggacccctactatatcaataagaaaacgtttatagtattgaataaaggg |
| | aaagcaatctctcgattcagtgccacccctgccctttacattttaactcccttcaaccctattagaaaattagctattaagattt |
| | tggtacattctttattcaatatgctcattatgtgcacgattcttaccaactgtgtatttatgaccatgagtaaccctccagactg |
| | gacaaagaatgtggagtatacctttacaggaatttatacttttgaatcacttattaaaatacttgcaaggggcttttgtttaga |
| | agatttcacatttttacgggatccatggaattggttggatttcacagtcattacttttgcatatgtgacagagtttgtggacctg |
| | ggcaatgtctcagcgttgagaacattcagagttctccgagcattgaaaacaatttcagtcattccaggcctgaagaccatt |
| | gtgggggccctgatccagtcagtgaagaagctttctgatgtcatgatcttgactgtgttctgtctaagcgtgtttgcgctaat |
| | aggattgcagttgttcatgggcaacctacgaaataaatgtttgcaatggcctccagataattcttcctttgaaataaatatca |
| | cttccttctttaacaattcattggatgggaatggtactactttcaataggacagtgagcatatttaactgggatgaatatattg |
| | aggataaaagtcacttttattttttagaggggcaaaatgatgctctgctttgtggcaacagctcagatgcaggccagtgtcc |
| | tgaaggatacatctgtgtgaaggctggtagaaaccccaactatggctacacgagctttgacacctttagttgggcctttttg |
| | tccttatttcgtctcatgactcaagacttctgggaaaacctttatcaactgacactacgtgctgctgggaaaacgtacatgat |
| | attttttgtgctggtcattttcttgggctcattctatctaataaatttgatcttggctgtggtggccatggcctatgaggaacag |
| | aatcaggccacattggaagaggctgaacagaaggaagctgaatttcagcagatgctcgaacagttgaaaaagcaaca |
| | agaagaagctcaggcggcagctgcagccgcatctgctgaatcaagagacttcagtggtgctggtgggataggagttttt |
| | tcagagagttcttcagtagcatctaagttgagctccaaaagtgaaaaagagctgaaaaacagaagaaagaaaaagaaa |
| | cagaaagaacagtctggagaagaagagaaaaatgacagagtccgaaaatcggaatctgaagacagcataagaagaa |
| | aaggtttccgtttttccttggaaggaagtaggctgacatatgaaaagagattttcttctccacaccagtccttactgagcatc |
| | cgtggctcccttttctctccaagacgcaacagtagggcgagccttttcagcttcagaggtcgagcaaaggacattggctc |
| | tgagaatgactttgctgatgatgagcacagcacctttgaggacaatgacagccgaagagactctctgttcgtgccgcac |
| | agacatggagaacggcgccacagcaatgtcagccaggccagccgtgcctccagggtgctccccatcctgcccatga |
| | atgggaagatgcatagcgctgtggactgcaatggtgtggtctccctggtcgggggcccttctaccctcacatctgctgg |
| | gcagctcctaccagagggcacaactactgaaacagaaataagaaagagacggtccagttcttatcatgtttccatggatt |
| | tattggaagatcctacatcaaggcaaagagcaatgagtatagccagtattttgaccaacaccatggaagaacttgaaga |
| | atccagacagaaatgcccaccatgctggtataaatttgctaatatgtgtttgatttgggactgttgtaaaccatggttaaagg |
| | tgaaacaccttgtcaacctggttgtaatggacccatttgttgacctggccatcaccatctgcattgtcttaaatacactcttca |
| | tggctatggagcactatcccatgacggagcagttcagcagtgtactgtctgttggaaacctggtcttcacagggatcttca |
| | cagcagaaatgtttctcaagataattgccatggatccatattattactttcaagaaggctggaatatttttgatggttttattgt |
| | gagccttagtttaatggaacttggtttggcaaatgtggaaggattgtcagttctccgatcattccggctgctccgagttttca |
| | agttggcaaaatcttggccaactctaaatatgctaattaagatcattggcaattctgtgggggctctaggaaacctcacctt |
| | ggtattggccatcatcgtcttcatttttgctgtggtcggcatgcagctctttggtaagagctacaaagaatgtgtctgcaaga |
| | tttccaatgattgtgaactcccacgctggcacatgcatgactttttccactccttcctgatcgtgttccgcgtgctgtgtgga |
| | gagtggatagagaccatgtgggactgtatggaggtcgctggccaaaccatgtgccttactgtcttcatgatggtcatggt |
| | gattggaaatctagtggttctgaacctcttcttggccttgcttttgagttccttcagttctgacaatcttgctgccactgatgat |
| | gataacgaaatgaataatctccagattgctgtgggaaggatgcagaaaggaatcgattttgttaaaagaaaaatacgtga |
| | atttattcagaaagcctttgttaggaagcagaaagctttagatgaaattaaaccgcttgaagatctaaataataaaaaagac |
| | agctgtatttccaaccataccaccatagaaataggcaaagacctcaattatctcaaagacggaaatggaactactagtgg |
| | cataggcagcagtgtagaaaaatatgtcgtggatgaaagtgattacatgtcatttataaacaaccctagcctcactgtgac |
| | agtaccaattgctgttggagaatctgactttgaaaatttaaatactgaagaattcagcagcgagtcagatatggaggaaag |
| | caaagagaagctaaatgcaactagttcatctgaaggcagcacggttgatattggagctcccgccgagggagaacagc |
| | ctgaggttgaacctgaggaatcccttgaacctgaagcctgttttacagaagactgtgtacggaagttcaagtgttgtcaga |
| | taagcatagaagaaggcaaagggaaactctggtggaatttgaggaaaacatgctataagatagtggagcacaattggtt |
| | cgaaaccttcattgtcttcatgattctgctgagcagtggggctctggcctttgaagatatatacattgagcagcgaaaaac |
| | cattaagaccatgttagaatatgctgacaaggttttcacttacatattcattctggaaatgctgctaaagtgggttgcatatg |
| | gttttcaagtgtattttaccaatgcctggtgctggctagacttcctgattgttgatgtctcactggttagcttaactgcaaatgc |
| | cttgggttactcagaacttggtgccatcaaatccctcagaacactaagagctctgaggccactgagagctttgtcccggtt |
| | tgaaggaatgagggttgttgtaaatgctcttttaggagccattccatctatcatgaatgtacttctggtttgtctgatcttttgg |
| | ctaatattcagtatcatgggagtgaatctctttgctggcaagttttaccattgtattaattacaccactggagagatgtttgatg |
| | taagcgtggtcaacaactacagtgagtgcaaagctctcattgagagcaatcaaactgccaggtggaaaaatgtgaaagt |
| | aaactttgataacgtaggacttggatatctgtctctacttcaagtagccacgtttaagggatggatggatattatgtatgcag |
| | ctgttgattcacgaaatgtagaattacaacccaagtatgaagacaacctgtacatgtatctttattttgtcatctttattatttttg |
| | gttcattctttaccttgaatcttttcattggtgtcatcatagataacttcaaccaacagaaaaagaagtttggaggtcaagaca |
| | tttttatgacagaagaacagaagaaatactacaatgcaatgaaaaaactgggttcaaagaaaccacaaaaacccatacc |
| | tcgacctgctaacaaattccaaggaatggtctttgattttgtaaccaaacaagtctttgatatcagcatcatgatcctcatctg |
| | ccttaacatggtcaccatgatggtggaaaccgatgaccagagtcaagaaatgacaaacattctgtactggattaatctgg |
| | tgtttattgttctgttcactggagaatgtgtgctgaaactgatctctcttcgttactactatttcactattggatggaatatttttga |
| | ttttgtggtggtcattctctccattgtaggaatgtttctggctgaactgatagaaaagtattttgtgtcccctaccctgttccga |
| | gtgatccgtcttgccaggattggccgaatcctacgtctgatcaaaggagcaaaggggatccgcacgctgctctttgcttt |
| | gatgatgtcccttcctgcgttgtttaacatcggcctccttcttttcctggtcatgttcatctacgccatctttgggatgtccaatt |
| | ttgcctatgttaagagggaagttgggatcgatgacatgttcaactttgagacctttggcaacagcatgatctgcctgttcca |
| | aattacaacctctgctggctgggatggattgctagcacctattcttaatagtggacctccagactgtgaccctgacaaaga |
| | tcaccctggaagctcagttaaaggagactgtgggaacccatctgttgggattttcttttttgtcagttacatcatcatatcctt |
| | cctggttgtggtgaacatgtacatcgcggtcatcctggagaacttcagtgttgctactgaagaaagtgcagagcctctga |
| | gtgaggatgactttgagatgttctatgaggtttgggagaagtttgatcccgatgcgacccagtttatagagtttgccaaact |
| | ttctgattttgcagatgccctggatcctcctcttctcatagcaaaacccaacaaagtccagctcattgccatggatctgccc |
| | atggtgagtggtgaccggatccactgtcttgacatcttatttgcttttacaaagcgtgttttgggtgagagtggagagatgg |
| | atgcccttcgaatacagatggaagagcgattcatggcatcaaacccctccaaagtctcttatgagcccattacgaccacg |
| | ttgaaacgcaaacaagaggaggtgtctgctattattatccagagggcttacagacgctacctcttgaagcaaaaagttaa |
| | aaaggtatcaagtatatacaagaaagacaaaggcaaagaatgtgatggaacacccatcaaagaagatactctcattgat |
| | aaactgaatgagaattcaactccagagaaaaccgatatgacgccttccaccacgtctccaccctcgtatgatagtgtgac |
| | caaaccagaaaaagaaaaatttgaaaaagacaaatcagaaaaggaagacaaagggaaagatatcagggaaagtaaa |
| | aagtaaaaagaaaccaagaattttccattttgtgatcaattgtttacagcccgtgatggtgatgtgtttgtgtcaacaggact |
| | cccacaggaggtctatgccaaactgactgtttttacaaatgtatacttaaggtcagtgcctataacaagacagagacctct |
| | ggtcagcaaactggaactcagtaaactggagaaatagtatcgatgggaggtttctattttcacaaccagctgacactgct |
| | gaagagcagaggcgtaatggctactcagacgataggaaccaatttaaaggggggagggaagttaaatttttatgtaaat |
| | tcaacatgtgacacttgataatagtaattgtcaccagtgtttatgttttaactgccacacctgccatatttttacaaaacgtgtg |
| | ctgtgaatttatcacttttctttttaattcacaggttgtttactattatatgtgactatttttgtaaatgggtttgtgtttggggagag |
| | ggattaaagggagggaattctacatttctctattgtattgtataactggatatattttaaatggaggcatgctgcaattctcatt |
| | cacacataaaaaaatcacatcacaaaagggaagagtttacttcttgtttcaggatgtttttagatttttgaggtgcttaaatag |
| | ctattcgtatttttaaggtgtctcatccagaaaaaatttaatgtgcctgtaaatgttccatagaatcacaagcattaaagagtt |
| | gttttatttttacataacccattaaatgtacatgtatatatgtatatatgtatatgtgcgtgtatatacatatatatgtatacacaca |
| | tgcacacacagagatatacacataccattacattgtcattcacagtcccagcagcatgactatcacatttttgataagtgtc |
| | ctttggcataaaataaaaatatcctatcagtcctttctaagaagcctgaattgaccaaaaaacatccccaccaccactttata |
| | aagttgattctgctttatcctgcagtattgtttagccatcttctgctcttggtaaggttgacatagtatatgtcaatttaaaaaat |
| | aaaagtctgctttgtaaatagtaattttacccagtggtgcatgtttgagcaaacaaaaatgatgatttaagcacactacttatt |
| | gcatcaaatatgtaccacagtaagtatagtttgcaagctttcaacaggtaatatgatgtaattggttccattatagtttgaagc |
| | tgtcactgctgcatgtttatcttgcctatgctgctgtatcttattccttccactgttcagaagtctaatatgggaagccatatatc |
| | agtggtaaagtgaagcaaattgttctaccaagacctcattcttcatgtcattaagcaataggttgcagcaaacaaggaaga |
| | gcttcttgctttttattcttccaaccttaattgaacactcaatgatgaaaagcccgactgtacaaacatgttgcaagctgctta |
| | aatctgtttaaaatatatggttagagttttctaagaaaatataaatactgtaaaaagttcattttattttatttttcagccttttgtac |
| | gtaaaatgagaaattaaaagtatcttcaggtggatgtcacagtcactattgttagtttctgttcctagcacttttaaattgaag |
| | cacttcacaaaataagaagcaaggactaggatgcagtgtaggtttctgcttttttattagtactgtaaacttgcacacatttc |
| | aatgtgaaacaaatctcaaactgagttcaatgtttatttgctttcaatagtaatgccttatcattgaaagaggcttaaagaaaa |
| | aaaaaatcagctgatactcttggcattgcttgaatccaatgtttccacctagtctttttattcagtaatcatcagtcttttccaat |
| | gtttgtttacacagatagatcttattgacccatatggcactagaactgtatcagatataatatgggatcccagctttttttcctc |
| | tcccacaaaaccaggtagtgaagttatattaccagttacagcaaaatactttgtgtttcacaagcaacaataaatgtagatt |
| | ctttatactgaagctattgacttgtagtgtgttggtgaaatgcatgcaggaaaatgctgttaccataaagaacggtaaacca |
| | cattacaatcaagccaaaagaataaaggtttcgcttttgtttttgtatttaattgttgtctttgtttctatctttgaaatgccatttaa |
| | aggtagatttctatcatgtaaaaataatctatctgaaaaacaaatgtaaagaacacacattaattactataattcatctttcaat |
| | tttttcatggaatggaagttaattaagaagagtgtattggataactactttaatattggccaaaaagctagatatggcatcag |
| | gtagactagtggaaagttacaaaaattaataaaaaattgactaaca |
| 2 | ttttttaaagaacataatgtagttgagagtgtaatgtactaaataacatctcttttttggaaggctacttttttttgagatagggtc |
| | ttgctttgtcactcagggttgagtagaatgatgtgatcgtggctcactgcagcctctacctactgggcttaagcaatcctcc |
| | cacctcagcctcctttgtagctgggactacaggcatgtgccaccatgcccagtgaattttcgtatttttttttgtagagagga |
| | gattttgccatcttgcccaggctggcctcgaactcctgggctcaagtgatctgcctccttggcctcccaaagtgctgggat |
| | tacaggcatgagccaccatgcccagcctaaaaggccacattttttaaccacattttcaaatgatcaagaaaatgaatatac |
| | taaagactcagaatggtttaatttcctgtgaaaaattggaatttttagaaatccatttcccaataaagagtatcttgtgattttcc |
| | aacatcactatagacatatgactttcaatattgctttttgataatgcatataagcacaatgaaaatagtaaataactgtagtac |
| | atggttatcataggaaatggaacaaaaaatttaaaagcattcttacctctttgctttcttctagttctgactcactgctgaactc |
| | ttcagtatttaagttttcaaagtcagactctccaacagcaattggcactgtgacggtgaggctggggttgtttatgaatgac |
| | atataatcattttcatcgattacgtatttttcaacactgcttccagtacctacaccactggtggttccattcccatctctaagata |
| | attaagctctttgcttatttcaattccagtattattggacatgcagctgtctatcttattgccttcatggatttctataacttttggct |
| | ttctaaaaaaggctttttggaaacactcccgcatcttatttttcacataatcaattcccttttgcattcttcctactgcaatctgca |
| | gattattcatttcattgtcatcatcagtagcagcaaggttgtctgagctaaatgaactcaacaataaggccagaaagaggtt |
| | cagaacctaaaagaaaaaaagaaaaatgtctattttaatattgaaatatgcatttaaataacagcctaaaaagggaactca |
| | tagatctttgctaaaattatttcaacaaatgtttattgaatgcttactatatttcagatgccatgtctaagtcacacactctttaaa |
| | ggacctaatatcctgctctaagggcaaaatgataaaacaagcacctcttctttttttcctattgggagggacaataggaaa |
| | gcagagcttctaagatttaaatggtgtctactcaggtttgccagtgcaaagacaaaatcctaccctcaaggattctgccaa |
| | cgagagagagagagagagagagagaaagatactttcttcttaatcattttatcttacttcatcttaccattttttttcctaggg |
| | aatttctagcttcgtgtatataaagaatgaacttatttatttaatagcctgatgatatgttccaaatgatatacttgaaatacaaa |
| | ttcctttggcccaatggccacaaacttttataacagcagaaatttctaagtgcaaattatatacgatttaacacgtttcccatc |
| | aaatacactttgaaatttatatgttcaaatagctcaacagatggagagaaatctaaaagtataccagggtttaggctagacc |
| | cacataagttacttttctttttctttctttctttgtgtgtatgtgtgtgtgtgtgtgtagaaagtatgaggcgattttaactttcagaa |
| | tatcatgggatatgtgaaaaagttactcagccaactgataagttatggttaatgcaggaataattcattatttctgatgtaaaa |
| | gaaaatttataataatatttttagcctgaaatgtagttagttattgagtttcagaatatgttacactataggaagacatgcaag |
| | gcaaaagccccagtttgcttccttacttctgtatcagattttttcagaatctagagctccctaatttactattctggagaaaaaa |
| | attacatgttcgtaggtatcccagtagcattcttttgaacaagtttatctgaaatgtatccctcaaaggaaaggcccaatatg |
| | atggcataaagaaatgctttgcccacgcatgactgagaaatttaagcattaaacatataaatatataaactatgcttttgcat |
| | tagtgattttaagtcacagttaaaagtaactcctttgagtctggacaatgtccatacatatgaataatttaaactgtaataattg |
| | gttttcctatagtaatgcaaattcagggaagaaaacattttgtcctcttccaagttacttggataattacaatgcatagctaaa |
| | atataatggtctacttcattgaaatgtgtcttcaaaatttggtactatgcaacctatgtctctaagtaagcaaactacatgaact |
| | atgaacatgcaaagagcaatttgattatagaatagatagttgtctttttagatttagtagaggtcattttcttatcaaaacaata |
| | ttaggatacaattcttcttagtgataaaaatttactttctttgaagaagaaaggaaaatctaatatttatgcccccaggcattca |
| | taaatacatctgatttaatctttgcattaaagatatgatttcagttttattattccaatgtcataaaaaatgggaaaccagagcc |
| | caggagaagttaaataacttgcttattctctgagtgctaatagggggatttcagtctaggccacttactataaaccagagat |
| | atgtttactacatctggccacgttcctagctactgtctgctcttgttctaatttatgagcatttgtactacatacataccacaag |
| | gtttccaatgaccatgaccaacatgaaaacaataaggcacatggtttggccagcgacctccatacagtcccacatggtct |
| | ctatccactctccacacagcacgcggaacacaatcaggaaggagtggaagaagtcgttcatgtgccaccgtgggagc |
| | gtacagtcatcattgatcttgcagacacattctttgtagctcttaccaaagagctgcatgccgaccacagcaaaaatgaag |
| | acgatgatggccaacaccaaggtgaggtttcctagagcccccacagaattgccaatgatcttaattagcatatttagtgtg |
| | ggccaggattttgccaacttgaaaactctaagctgtaatcaagtgaaaagatgctgttagtagtaatcataatataattttag |
| | acattattttattagtatgtggtatcatagaaccacacgtggtagatgtaaaaactcaaaatatactgaactgattcaatttca |
| | aatccagaaaaaatagtgttaacaatattagaattgtaaatctggtcataatatcatttaagtgcaaaaataaaaataaaaca |
| | tttcagaaataataaataaaacatttctgattcatccaaagatatgcccatgtaaacaaatggtattttgattagagacttttta |
| | aattagcaaataaatgttatcttcagattctattttagacagtaaaaagaaatattaaaagtataaatcatagatttttataggc |
| | caatataaaagtaaaataacctaaataatgtgataaaaatgtacaggaaaaaaattgaaaaaatacacattttgcttaatttt |
| | caataaattttacatatttctcaataaaattttgtgataattcaaaaagttttttatacaattatttaagatactaataacatcaaat |
| | attttcccacatgttgctttaaaacagtccccattcctagaagtagggttaataaaaagctaaaattaactggggtctaaattt |
| | ttatttgcgtatctttacagactgtaaggccaaaaaagcttcataaacttaaaacacagttttagaaagtacttgtaggttttct |
| | aatgtgccctacatacctaatcattatagagtattttgaatgctttcatcattcttagaaaatggtttagctttaaagaggatgtt |
| | tacatgttttcttagaatgtcacatattgatgtaaatttaaaattaaaattaataattatataaatatacattgattcaatttcaaaa |
| | taaatgttgtgccaatgagcgacagggatatatataaatagataccagtctgaatgatcgcagtacagacaatccctcca |
| | catttgacagaccaagctccattaaactgaggctgacaataattccatcaaagatattccagccttcttggaaatagtaata |
| | aggatccatggcaatgatcttgagaaccatttctgctgtgaaaatcccagtaaagacctaaaaaatagagatcagcacta |
| | cttcaagagcactgaaaaacactgatgctacacgaatttataaagaagaacattaacaggaataaaaatatctaaaattta |
| | aagtgagagaatgtgacaaatcaattcaagtcttagtttttttattttcattttttatttttatttatttttatttattttattttatt |
| | attattatactttaagtttgagggtacatgtgtacaatgtgcaggttagttacatatgtatacctgtgccatgctggtgtgctgcaccc |
| | attaactcgtcatttagcattaggtatatctcctaatgctatccctcccccctcccccgaccccacaacagtccccagagtg |
| | tgatgttccccttcctgtgtccatgtgttctcattattcaattcccacttatgagtgagaacatgtggtgtttggttttttgtctttg |
| | tgatagtttactgagaatgatgatttccaatttcatccatgttcctccaaaggacatgaactcatcattttttatggctgcatag |
| | tattccatggtgtatatgtgccacattttcttaatccagtctatcattgttggacatttgggttggttccaagtaattgcttcaaa |
| | gagaataaaatacctaggagtccaacttacaagggacgtgaaggacctcttcaaggagaactacaaaccactgctcaa |
| | tgaaataaaagaggatacaaacaaatggaagaacattcgatgttcatgggtaggaagaatcaatatcgtgaaaatggcc |
| | atactgcccaaggtaatttatagattcaatggcatccccatcaagctaccaatgactttcttcacagaattggaaaaaacta |
| | ctttaaagttcatatggaaccaaaaaagagcctgcatcgcccagtcaatcctaagccaaaagaacaaagctggaggcat |
| | cacactacctgacttcaaactatactacaaggctacagtaaccaaaacagcatggtacttgtaccaaaacagagatatag |
| | atcaatggaacagaacagagccctcagaaataatgccacgtatctacaactgtctaatctttgacaaatctgagaaaaac |
| | aagcaatgggaaaaggattccctatttaataaatggtgctgggaaaactggctagccatatgtagaaagctgaaactgg |
| | atcccttccttacaccttatacaaaaattaattcaagatggattaaagacttaaaagttagacctaaagccataaaaacccta |
| | gaagaaaacctaggcattaccattcaggacataggcatgggcaaggacttcacgtctaaaacaccaaaagcaatggc |
| | gacaaaagccaaaattgacaaatgggatctaattaaactaaagagcttctgcacagcaaaagatactaccgtcagagtg |
| | aacaggcaacctacaaaatgggaggaaattttcgcaacctactcatctgacaaagggctaatatccagaatctacaatg |
| | aactcaaacaaatttacaagaaaaaaacaaacaaccccatcaacaagtgggtgaaagacatgaacagacacttctcaa |
| | aagaagacatttatgcagccaaaaaacacatgaaaaaatgctcaccatcactggccatcagagaaatgcaaatcaaaa |
| | ccacaatgagataccatctcacaccagttagaatggcaatcattaaaaagttaggaaacaacaggtgctggagaggatg |
| | tggagaaataggaacacttttacactgtttgtgggactgtaaactagttcaaccaatgtggaagtcagtgtggcgattcctc |
| | agggatctagaactagaaataccatttgacccagccatcccattactgggtatatacccaaaggactataaatcatgctgc |
| | tataaagacacatgcacacgtatgtttattttcattttttatttttattatttttttttgagacagagtctccctctgttgcccagatt |
| | ggagtagagtggcgcgatcttggctcactgcttcaagtgattctccagtctcagcctcccaggtagctgggattacaggc |
| | atgggctacctaccatgcccagctaatttttctaatttctgtagagacagggtttcacatcacgttggccaggccagtttcg |
| | aactcctgacctcaagtgatctgcccgcctcagcctcccaaagtactgggattacaagcatgagccaacacacccagc |
| | catgtctagcttttttttttttttcctctaggagagtattttattaagtagagtggtattatttaatgaatcttctggatttccaagaa |
| | aaacactaattttaaaacatttcatggtctacattttaactctggtattgaataatagctactgaattattgcccttgttggtaca |
| | ctacatttctaacataatcacaaccaaatttatttctgggtagtatcgcatcaggaacaaatccacatattactgaaagttatt |
| | aaaaattgatttcgtaaaaaccatattttgtttttgattttgtgttttttaagggacaccatcttatttctataattttataaacattcc |
| | tgaaacaaaagtaagtttgccacaaattgtttattataatgatctctggatgtatgcctcagaattaaggttaatggattaata |
| | gagcaagaagctgcagatttaattgaaaccagcaggagaaaatactcagtatgcatatacatgtaattgagattaataagt |
| | cacttgagtggctagataaatgtgtatttgacaatagaaagaaaaacaatgaaacaaacacaacatttattttttaactcaa |
| | aacgataggctgagagatacaggacatgaagaattatgaaattactcttgcagaaaatcttttaaagcaattctcttaatttg |
| | ctatagttacaagttgcagtttatgactgtaaagtcaagtttagcatttacgaagataaatcagcctttatcttgaaggaattct |
| | gtcaaaataacagtataacaattatttggttccacttaaaagaaatcttgatccacccttacgaatatataaatgcagctaac |
| | acttcattttttccaatcacagatgggaaatacccatcacagaaaaaaacagcctaatggtagtcatccattcactcaaata |
| | gaaataaaatgtattttaagcaagcctagtacatgaaaatccaagtatatggtgaaatgcccattgaaaaatatatttttcatt |
| | tatagaataacacatcaaagattttttttttcaaatagtgagtttgcctggtaaatgtaaaataagttgtaactatacaaattttat |
| | ttcatgtataaaacttttagaagcagctattatttgaaataggcactctgatgatagaaatatattatttaaggtgagctagaa |
| | ccatccaggtaaataatctacaaatatctctttctatagaaaatcataaggtgatcttttcagctcttctaaaatgactagatct |
| | catacctttaaaaacatataccctaaatttacgcttatctaaaaatgtaatcccgccataaaacttttcattaaaaataatatttt |
| | tattacaactagcaatccaaaatgacagctatatctctgtaactgttttcagttaaggccctaaaaatcaatgggattgttttt |
| | ggtacattttaatctagtttcagcattgtcaggtaagttggaaatgtttgttatgatgttgtctaacctactatgattgataggct |
| | attttttgctgattgaatccctatgatgggagcaagcattttataatgattaaaaaacaaaacaaaacaaaatactccacaaa |
| | aatacggagcaccttagcaaaacttaattgagattcaaggtctcttgatactcaaatttgattaatctcatcaacaaggaagt |
| | gatgtaatttttatgtactgcagaaacagaatgcaaaatggggcatattgatctaagttaagatggaaaattccacaattca |
| | gtgtcctcatgtagaacataaagcagtatgcaataatttgagcagtaataaactagtctgtagatattctaaatttattttaatg |
| | agcaaattataagctctccacaaaacattctgtgtaatcatctatgatctataatatagtcctaagtcagcttagcaccaatg |
| | atagaaattctttctttctctctcacatgctctttctctgcatatatattaaacatatgtattactttcatatggatatttatgataaa |
| | ggttgttatgggtaggaaattctatgggtagatattctatgaacaaatgctttgtaaattatgcaatgcattattgatatgttaat |
| | cattatagtaaggaggtatcaaccaatagagacagtcagtttacatattctcctatttttatggatagaaataaataactcaa |
| | gtctgttccctataatttatgctctctatcttggcaagttttgtttccccaacactaacctgaacttatagatggttctatcacaa |
| | acaatctaccaggcatcagtctcccattccactttcccacctcattatttcagttactacccataaagtcatagaattgtagaa |
| | ctgggacatacataaaaagtcatttagtccaacacactcattgtttatatgtgtgttgagaaaatagactcaaggatctaagt |
| | gacttttcaaaaccacacagatatttaccatgaacactttgactaggacctagatttgatgcttttgtctagtgctttttcattca |
| | atcatcttataatccagcctctcttatcctgtgtttccatactcaactgcttcagcattggaattctatatttcctgcttcactctg |
| | gcaaatttccttcacctgttcaacttccctaactgtccaatatcctgggaaaaatacaagaactacaccagaaatattttcca |
| | tcattatttcaaagaaaggtctaaaaggttaatagcatatatatgaggaaggaaactgtttattgagcacatgctagatgcc |
| | aggcacagtctaggtgctttcacattacctcatgtgaccttcacaacaactctgcacgaagggtattattacactcatttcaa |
| | taatagaaaacaaagattcagattgtttatcagaattgtccagaggccatatcactagtaagcactaggcaactaaggtca |
| | aactcaattccactccactgcaaagtctgtgctcttttgcaaaatcatgctacttgaagcagcctttttgggtagttatcaacc |
| | agtgggttatttattgagtgactagcatgtaaacactgagagaggcaacagaaaaatgggacacggcctttgccaaaga |
| | tgaagaaaatctcaagaatatggtgggtgttctactgagtcaaataatgcagataatttgaggtcacttattagggcacac |
| | aattatccaattgtccaccagcatttcctttcccccttctgttggcaattgctccttcacgcatgattcctcaggactgccatgt |
| | tttcaacatgatcccaccctctgccaaggctgattaaatcagaggtgagcatgtgacctaaattatgcctaagttctaccag |
| | gagagtctttggaatgagactcaaaaggagattcatgcttttcagtgtagaggttgtaggatggaaaatcagtgagctctt |
| | gctgaacatttgctgacctaaagacaaatgatgcagtgaacttgtgggccatatctaagcccttgatgaaaactgttccta |
| | aagtcagactgcaatactgccctttacttagtttgttagtctaccttttccctccattccagtttccccactaccccttgcccca |
| | agtttgttttagtacagagaaagctcttgattaataatatatgtaaacccttttcttacttgttcacccattttgattagcacacat |
| | agacacagatgaatgctcagtaatttgaaccagaggtatggctaaatcacaaatgcaaatttagcaactattttttttcttgta |
| | tgattatcactgaagctttttattaggtagttaacttgaaacaatgatgttctttttttcatatacatcaatctttgggtcatccctt |
| | caatggacatgattgttttagaggctgtgtgggttaaaaccacatgattgtggagccagtattagatctcctggtttatctgg |
| | acccacatttccagcaagaacaactgtaaagattttgagacgtttacaactggtaagacaccatgatcaatcagcacaga |
| | cattggcctaaacaatcagcattcataccagttgagttttaggctgtatctctgagattccctgtaatattccttagatatttctt |
| | tctatgaggaaagaggatataatttttgggttcaacacagcacaaatacatctttccctttggtctatctctcccacatctacc |
| | aaagtaaataagtaaacttcaaatgtacttaccaggtttcctacagtcaacacactactgaattgctcagtcatggggtagt |
| | gctccatggccataaagagggtatttaagacaatgcaaatagtgatggcaagatcaacaaatggatccataacaattaaa |
| | ttcacaagatgttttacttttaaccatgcatcacagcagtcccagatcaagaacacattggcaaatctataccagcatggc |
| | ggacatttctgtctagattcttcaagttctggagggacaataacaaggaagagtagtaaataacaacaaaaatgagttatt |
| | attgctagtagattttacattatttaacaaaattgatgcttatatttgaaaatagaagttcatattacttaagaaacttgagaaga |
| | caatcccattaaatattttcagaaaatgttaattaaaatgtcatatagatcgacttttttctcttttgaatacagaaaataagcac |
| | tttgaatttaatattttgcttttaaaatgactaggttatgattgattaaagtgctgtattctctttgtcaaaacagaattttaaataa |
| | acattacacaactattaaaattttaaataattattgtttcttctgaaagtacctttttaaaaacattgtctaaagttgtgatccctta |
| | tgtgtgtgataacatttatgaactattctctcctctcatagtttccatatcaacattaatcttgcacacagtgagaacgtagtat |
| | gcctttctttttagtataaattcagactcaaatctacttttgtggtttgtacatatcttttagaattttagcatcatgtatttaaaatat |
| | tgcttgaaattttaatggatgaggcctgttacaaaataattattatgtgactgtatatctaatacaccaagaagacatgatata |
| | aaatgtaattgatgttttctaaagaacatatgttagaattgacatataattgatctaaaaagggcagtactaatatattctctct |
| | ccagacttcaaggagtgagggctgtgtgatgtatgaacaagtttagtgtataaatagttgctgcctattcctctgtgtcaaa |
| | atcaacaaacatttctttccgtggtcctttggaggaatgtaatggttgctgtgtatgtagaagatggaagagagacccttttg |
| | taggatctctcttgcagtggaggacaggttttgatttgcccaactcatttaagtgctcccgcaaccccatcaacacttccat |
| | gcctgaagaacagactaaagcctgaaagaaatgtttccttatgcttctatataccaagacttagaaaaggggaagagttta |
| | ctctatttgcatatgagtaacataacataagaataaatcctcattgatataatatcaagtgttgaaatatcactctgcaagtaa |
| | ggtaaggacccaagtcaaaaagaaataaggccaatattagtttttgtaatcaatgtgataatgtactccatatatcacattct |
| | taggtctaatatatagtttcgatctgggtaacagacttcagtaattctatgaaaagaaggaatagttcacaaagcacataat |
| | cacagaaagttggctgttccatgacctgcttcttaccttccattgtgttggtcagaatgctggctatgctcacggctctttgc |
| | cttccagaggaatcctccagcatctccattgaaatctggtaagagcttaaccttctctttctgacttccgtttctgtggtggtg |
| | ccctgcaaaccaaatactgatggctcaaaccactcttcccaataagtaatacaacaccacatagcatttctttggcaaatc |
| | atgctacatgcaatttttccaggtaagaattttcaaggaataaaatattgaattattgctaagttataaacagttttttttttaaaa |
| | aaaagttatcacaggtagtactctttaagaggaattcaccagtgggatgcagttttataacacaattatattacagaactattt |
| | taattaacctgtcacaattttcaaggaaatttgttgtaaacatgctaaagtcatttattattttatttcattcaggtgaaaattatttt |
| | tatcttctatcaatgattaataatcagatcaatctccccccaataaatattatgattatatagtctcaaggctgaattattttcttt |
| | gaggctatattgacaatattgacatatgtgggccaattttttccatgcacaaacacagatgtacgatgaaatgtgtgtgcttt |
| | cctgaaatttctttaattatttctatatcttatcctattaaaaaacaacacattttagagtcattttaaaatagcactgtaaatattg |
| | acagttgtatgaaattgcccattatcaacaaattggttataatatttaattttaagaatatgaattttccagaatatgaatagata |
| | attatacaattcttggtgaatatacatcttatagatttgatggtgaaaatgtccattatttgtctcggtaggtctaccaaatttgc |
| | agatatgtatttcatagcagactctaaaaacggggcccttttaacctaaagccatggtcccttacaagtaattcccatacaa |
| | caaaagctaatggtcccatacaattcaattgattctcaatattctattgttttccctttgattaatcatgtattatttttaccaacttt |
| | cattttgaggaagcaggacggtatgacagcctaaactgtccaggctttgattatttcaaattggtgaataatgtcagtagca |
| | gctaggtcatctattatcacctctgggggaagttgtccagtaggtgacgttagagctgaaggtccacccaccaaggaaa |
| | ccacaccattgcaatccacagtgctgtgcatcttcccatttgctggaagccctggcaccatcctggatgacatactggcct |
| | gactaacgttactgttgcgtcgctctccatgtctgtgcggcacaaacagtgagtctctcctgctttcgctgtcttcaaatgtg |
| | ctgtgttcatcatcagcaaagtcattttcagatccaacatcctttgcccgacctctgaaactgaaaatgcttgttttgctattg |
| | cgtcttggggaaaacagggagccacggatactcaagagagactgcagagaaagcaaaaaggaaaggaatgggatg |
| | ggggtaggggaagaacgcaattatttaataaatattaatattaaaggatctgttttctttggagttagtatactcttgctcttaa |
| | acatggataatcaaatttatagaggacacatatattttgatatttttattaaagcaaatttaagtacaaagagtgtttttctgact |
| | aacaatactgatttgcaatttactaagtatacaaccacttcatttagtcttcactttaggtggattgggttatcataatgctatag |
| | gttagggaccagatgctgaaaaggataaatgatttcttttgatgggaatggtggttaaacaaatagtaagtagcttctactt |
| | cttaatgctataatctgaagggaatgtagcctggagaataagttttggctcaagtgagaatgttagctcaaatttaaaatatt |
| | tgtttcaaatgttgcattgtagccaatatttcacttcaggataattacttttttaaaaaactactatttttccattctcttcttttgaat |
| | tctgaaagaaaagtagtttactctttctctgaaactgtttgtctctacccacatgggaacagaagtcacaaaacttacctacg |
| | atgttgtcttccaaatttgctaacagtttcccaaatggcaaccaatgattcaccttatttgagagactatatgtcatagcaaac |
| | acaacacagaggattcctaaactgctacacaaattactcttcttgtgtaaaatataacactggcttgtaaaagcaagacata |
| | taacatgacttcaccagcaggtggtgtgaaaaacaacatatacttctgttttattttaattatattaaaaagaaagagacaatt |
| | caaaacctttttcattacagctattcccagataaattaagcaaactatttcctagtaccagaaaatgaggatttagcttgaagt |
| | tccttctttcacattttgaaaggattttaaactagcaaccccaaataattctgtgtgtattcttgtcttgcagatagttttcaaaaa |
| | catctctattcagtctgcagaatatttgttttctcccctcatcatgaacaatggcattcctccttgctctctcattccctttcctac |
| | ttctcttcacacccagggtaatatacatacatacatatacatatacatatgtcagcagcaaacattcctggatcttctctttctt |
| | ctaagctttagtgtaaaaccaaataaaatgattttagtggaaaaaacagaaaaggcagactctgtcaaatgtattcttggtt |
| | cctctgcctaattaggtcatgctgattttcttaggccccaatttttagctttattttcatggcttttcccagactgcaaagcacca |
| | gaataatcttttgttgtcctctgtctcagtgatattctggaaggatattattcactaagaagtgctctgttcctttggatttggtta |
| | gattctccacaggaactattggctgtccagcttgtccatcggaggctgtgtctatgtgacactcaattcctgtagcaggttt |
| | gtctaccgaagggcagacaatagagtgattttacagcaaagtgatcaagagattagatcatgtgaagacatcaattcatc |
| | cccccattaataaagaatgctaatgtttttcatttcctcagactccatttaaatgatcaatataataacagttatatccagaact |
| | cgtgttgttgttgttgttgttgttgttgttgttgttgttgttgttttgaggcagagtttcgctctgtcgcccaggctggagtgcag |
| | tggtacaatctcggctcactgcaatctccacctcccaagttcaagctattctcctgccttaccctcctgagttggctgggac |
| | tagaggtgcccgccaccacacctggctaagttttgcatttttaatagagacagggtttcaccatgatggtcaagctggcct |
| | cgaactcccaacctcaggtgatctgcccgccctggcctcccaaagccctgagattacaggcatgagccaccatgccca |
| | gcccagaactagtgttttgaaacatttgagtgtatgtgaatcactctgagagttaaaaatatatatttttaggtcccagattttg |
| | gaatatggattcaggaagtagcagataggactctggaatctgaattttcctgacacattccaggtgaccctaacacaggg |
| | tggacctcatcctacactcagagaaatactaacttggtcttacattaatccctaggatggctgagtataatcacctcaatac |
| | acctatgttctgaaactgtctctggaaactgaaaatcggtctacacagtattggttgggagtatgtggtacgtgtgtacatc |
| | cactatgtatctgggacatgcctttaatttggatatttttatgtcaggtattttcaaaggcgttccagatctctgagtttatttgaa |
| | atttctattcactctttggaatgtataataatgggtgtgagcgcagctttcaaggcaacagagacggaaaaagaagataac |
| | agaaatagcattcagagaaaataaaccaaaaataacacactagaagatgcagcagagtggagggaatacccacagct |
| | gtggcaacactttctctagggaaaaccagctttaatgttttaaggagtgaaaggccatctttctttacctcaggcatgtcaa |
| | aacatatttcccaggtatctttttcatcttgttcccagaagataaatgtagactgtttcttaattttcagtaacaaaagggaaaa |
| | tagaagaaattaggtctataagacacagtcatgcatgtagggtttggattcaaagtgggttgggaaaagtcaagatggaa |
| | aaagatgctgggaattttaagatgttctagtgggaacaaggaaagtgaatatttgtcagaagtacttagcattttgaggtttc |
| | attttatacttttaagtatttctttattttgtacaactaacattatctgtttaaaaataaaataatgtaataaacataaaataacattt |
| | gtaatttatgtgaatagaaggcagggcacccacaatttatgccctctctgctttgacattttcagaagatacatttgatttgag |
| | cagtatttactgtctatcttacgcttcctgtccccaccaccttcactgattctctttctaccagattccttttttgtttacagaagc |
| | agaaattgtaaaccgtttatggtacatttctgcttgttagttttgttaatgctcttagtctgtctttactgggatatgcaaatttaa |
| | aactaatagattatttccttcacttttgaaattatccatctatcttctgtctatttacaaatataaaatgagcgacatctaccacat |
| | gtatgtactgattctgcttttagtattaaaacactggccatcagctaagctctctgctcttgcctgtctgtggttcactcagctc |
| | cagcaaaccaagtctaccagttgcttcttaagcactccaggcatgctaccatgtcagggttttgcacttcctctttcatcagt |
| | ttagaatgctgttccttaagagagcctctttctttgcctcctccaaatctttcccaaaatgtcttaatagtgaggtcattgtaaa |
| | ccaccctatttaaaattttacctccaattatggctcctgttcctcttttcctgcttacttttttttcccttaacatttgttaccttctaa |
| | cacagtctataacttatttattttattcattgtccgtctcctttggaaaaaatttccatgaggtcagtggattgagtctcttttattc |
| | tttgaggtattttcagcatcttgaactatgcttgctacatagttggcgatcaataaatatttgttgattggactgaatgaactga |
| | tctacaaaatttatttgagcaaaatgatgaaacaataaaggaactaatactgctatcaaattatgtatatacatttgttagtaat |
| | tgaaactactcagaagtttgctttccctcacttgaaattcattcaccgattttaacttaataagagacttactctttgtcacaca |
| | gattttctatatattgcttagtcccctaaatctctttaatgtaggtattcttactttattttacagaaattttacaaagtacaagtga |
| | cttgaccaacgtcacagaactagtttggagttgagatatgaacacaaaagtttgtgttcactgtagcatcattatgctatagt |
| | ttcatatactcttaattctaataactcctaatagttttaagaattccaaaatatcaaaggtgactgtttaagaaaaaaaaatctg |
| | atctgaggaaccaagttgcccttcagagcttcaagttaattccaaatatgttatactgatttgataatatattttatatttgtatta |
| | cacaagatagtaatagttgaatttcttgatattgatgttatctgcagaaaaatggctttatcttaaaactctttgtgtattcatgtc |
| | cattttgttctaatatgagaatacaagttttaagttattaatatagtctacttttttctttaaaaatatttgtttacatattaaagatag |
| | attataaaacattcactgagaacttgcatattaccttttctcccaaccaaaagtaaatactatgttgaattttgtgtttattacttc |
| | tggttttttttagaagtttctttctttatgaaaaatatatcataccccttggttgatttgcagtttttgattaaattaatcttatatatt |
| | atctctcaaagcattatttttagggtataattctgactcaagattgttatgattagaagcatttgcaattcatagcacatgaaattc |
| | taatggtgccaaatagtgttcaaagccaaaattgtacttatcacagattttgtaactaattattctttacatcagacatatgtaa |
| | acaatacattaacttactaatccataaaacttactaaacatcaaataattgaaactagtttttactcttttacttaaaactatgct |
| | gaagaaccaggtagttggcattaaatcagagcaaaaatagactacctttgatgctgttctcttcaagaacatttaggtcata |
| | atttcattctaatgtatcacggcatgaccctatgcaagcaaaatggagactcaccatagtccagttctcttaatcaaaggat |
| | aaagacaaaacccaaagaacacttcttttcaaaatttatatgtatgttacgatcacaactttggataacaaaggcagaaac |
| | ccattctcaaagaaaataagtttatctggggtctcaaaatggaaaatgtaaacaagagattccaaaactctcattgaaaag |
| | tctgctagtgtaggttatatatatatatatgtgtgtgtgtgtgtgtgtgtgtgtgtgtgtgtgtgtatagacacatatatacacac |
| | atatatatatacaggttgatatataggttatatcaatatatattatatatatctatatattatatcaatatatctatctgtatatataat |
| | cagtctcaaaatggtgaacattttattaatggtttaataaaaatccctttgaagacaatctattataagaatttaaacaactata |
| | ttaaatgttagttccctgtccctcactgaagtcagatagtacaaaagtgtaccaaaacaaaatacaaaaactaaaaagcaa |
| | tagcaatgacaaagaaaccagagcacttagtagaaaatcatacctgatgaggggagcagaattttttgtcactggtcagt |
| | ctgtttccatccatggagaaaaggaagctgcttcttttgacgctgtcttcagattcggatttgggaaagctgtctctctctcct |
| | ttgttgtttccttcaaggtgctctctctgtcttcttttcttccttcggttcctccattctttagcacttttggaactcaactttgatgct |
| | tctgaagaactttccaacagctctcctaacccacctattccactgaaatctcttgaagcagctgatgctgccgcaactgcct |
| | gtcataaaacaaagccaggcactatttagaacacagagctttgaaaacagttgagtatggttaaaatattgccatttaaga |
| | ctcattaactacaagtgaaaagtctatcctctagtccttttattttgtctcaccacaagagtttacttaacaatgacccttatgtt |
| | ttaaatcatataaagcaatatgctagtgtatgcattaaagcaagaaaaatctggttttgcctatcttctggattgtcttttttttg |
| | gggggggggggttggtgacaactgttctcttcaaagtgtcttctatactttaaaaagcaaatgttttcttcgtggtggttgctt |
| | atctaactaccattctgattccatctgtaaaacgtttcctcctgtgtgcacttctgagcagctgagtcgtattttttcccctctaa |
| | atccagtacaaaagactgtttcttttattttttgtgaacataaaacatttcaagtgtctactattttacctccagatggaagtagg |
| | gaaatgtattagatagcttatcctcttccttcctttgaattacatttaggactgtagttcacatctctgtgatcatgttactccca |
| | gagaatttattacatatcacttaaaaaatgttaaacgttcatagattatttaatattataaataacaaatgattaacatagcatta |
| | cataaattcattcaaaggattatgagaactgcttcaaatattttctttctatattgcagcactaagtattcttaagcactttgaatt |
| | tctgtacctacaaatgaaacacagtcagctctgaggtgaaacatggcaaatagtatttttagactggaaatgatctaacaat |
| | gacatacagaggatatgaaacaatatggtgtgcatcaaagctgagttctcattaagtatattactatttgtaaatgtaaccac |
| | agaagtgaaaataattaaattatgacaatatcagttaaactttttacatatgtcatcactaaatacaaacaaatctaacttatct |
| | aatatattatctttcccattaactgtaagctagatagaaattgtatcattgtatgctcagtgactagtatgtcacctgacataaa |
| | taaggctcagtaaataattggtgaataaagatttaaaaatgtactttgctatcaattgtctcagacactgggggggggtgg |
| | gggaagaacaagacatacttaaccttgactttgatgcttctgaagaactttatagaacttatatttaacttgctacagatagc |
| | aagttgacttttcaagacatcataaaagacttgacttttcaagacatcataaaagactgtcataacagtctatataacaacat |
| | attttaggaagaaatggagataaaacctatttataatgctttgacaacactttgtgctttatgtttcatctttcttgagaagaag |
| | caaactctcctaagtagttttcatttttaaacttcatgtttaaaaatttaaaaaagattttaaatctaaaattactatgctgcaaatt |
| | ctctaggattacaatgatctcagggagggaaacttagtactaaatatgattttaaatttatgtgtttgaccaagcaacacata |
| | aaatgtaacatatttatttatattaagcactatattcataatggaagctcaaatctaagacttgatataagacaatacccagca |
| | tttaaataaaacagtaattcagaatttagatatttataaaattttatagagtcatatagccaggtttttaatggaattattgatcat |
| | cagtatcacttttaatataattcatgatgtaaaatgagaaatattaagtacattttataactctgactatatatgcaaaataaagt |
| | gaataaatgaatcacttatctatagcaagttaaatatagttacagagttctggatacatatgaggtacataatatacaccattt |
| | gttgaaacagtgaatatatatataccctttcttatccaaataaagtccttaattctcttttctattttatatagacacaaacacac |
| | aacacacagtgttaatcatttattgacttgtttgtccctgcaaacaaaggaagtggtggtaatagtcccttaccataatccta |
| | caacttatgtccatttaaatcacttccaaactttttttttttttttagatggggtctcactctgtcgcccaggctggagtgcaatg |
| | gcatgatctcggctcactgcaacctccacccccgggttcaagtgattctcctgcctcagccccctgagtagctaggatta |
| | caggcgtgagccaccacacctggctaatttttgtatttttagtagagacagggtttcactatgttggccaggctgatctcaa |
| | actcctggcctcaagtgacccacccacctcagcctcccaaagtgctgggattacaggcgcgagccaccgcacccggc |
| | cacaaactttgttttgcataagacattatttccaaacctattctgggtaacagttcaaataactcaaaaccaaaacagatttct |
| | aaggcaacaaaggtaacctcagacacattttctgtgtcactcatcaactcaaatctgtcatttgttccacttccagtgatttg |
| | ccagttccctcaaaggcagatgtttcctgtcatctctcatgtctgtgtacctgtttatacccaactctgtgacactaccaatgt |
| | cctgcgtatgagaataactgtgttactttcacagccaacttgctgaattcctcttttaacaaataagaaatttcacttaatattta |
| | ctcaggaaattacattttcttataacatttttgtgtgattcaaagtaggctctgtaactcactgtatctgtgtgtccgtaaaaata |
| | taaccacataactaatactttgttcattttatgtatataatccagttaaaatcccattcaaagctacaagacattatacctatttct |
| | gttctaccccaattatattagatttcttaagagctactatttcgaattttgttctagagcttactcccaaattttttcctatcagag |
| | caatatagactgctatgggatatactaagctgggcaacattaggcacttaaatatcacaatttaaatgtcaaacacaattag |
| | aatgatctaggacaaaacattgatttctttgcagggtttgtgttttctccacattatttctcatgcctacaccgtgactagccct |
| | aggtggcccctcaaccaataataaacaaagatggaagtgatgctagccttcatgaagtcattggctagatagtaacttttg |
| | aagagtcgtaggtggttagggagacttaagctacactgcagcataaatgatcttataggtgcactaattctgtctttgcctc |
| | cacttcctcctggcttcaacattgataatttactagtcatcattacttgtgttgtcaataaggaactaaaatggatttggcactg |
| | tgctactccaatcagtattatactttaccttggataaatttaaattttatatgtatgaagctaaagctgaatcaaattgtgatgat |
| | ttggtcactaagattcagtaactgactactaaggtaatagaactgatgttttttagccgaactcatcataatcataggttcttta |
| | aacttctactagaaatagtgcatgtttatttctaagttcaaaaagactatactctttttaattgatgtcatattttaccctaaaatg |
| | gctgcattttagaataaactcattatctgtgtaggattttcaagcacattaatttgtcttcttttattaatgataatagaaaaaccc |
| | agtgctttaaaatacaatcatatgcaatttatgaacattatttcctataacacttatttaaaaccttgaaataaagtatttgccct |
| | gtaaatagctatttttattttattatattataaatgtgttttatttatggtgacatagaataaaactcttaaataattcaaatgtagaa |
| | ctttctggcaagcatgaggtatattttaaaatattttcaagtttcaccaaatttgtttatagttttctgtataaaatcaataaggtat |
| | atccacattactactctgtatccaatggtaagatgaaagtggtcaagctagcatgttcagcaaaagctatgcctagagttg |
| | agctcaataattcactaaacaactcattgctgtatctccattaccccaaaaaatgccttgagaaaggacctaatgctatttgt |
| | tgtgcatcttcaaagtaactcagaattgttaaaaacttaaattttgaatataccaatttctgttatatactgtcatccaaagaata |
| | cttcaaatatgtggagccattcatctgaaaaaaatcctcaaaaaagtcagtgcataaaaatattatttggatttctgcttctgg |
| | ccaaaattgagtaacagagaccagatttgctctctcctgaagcaaccaataaaacagacaaaatacatgttacagtagttt |
| | tgaaggcactggacatcagatcaggatgttttaagatattggaaaatggaaaagcaagcaagtcagttctatgcttttgtta |
| | gcttatggcctggaacttccagactatgatgtagagaggaagtatctctgcagactccatgaatggatgagatgaagctg |
| | agtttaggaaaatcagggtggttgtatagttcacaaaacagggtaccacagaggagagagttgagcagagagagaact |
| | ctagggatcttccaggttccccttgagtactgagaagattattcaccaagcatgtgtcttagaaaactacctgaggctga |
| | ggataagagagaacaccacctggcaatcacactgtgccagaacagtgtctgttcctacactggcaaacacttcgttttaa |
| | atgcgcattgggtagagtattaggaagggtcttgattcactagtgaggaataattagtcttagaataaatggtatgctggtc |
| | ctgcctaacaaattttcaaatgtttaaagatacaaaaggatcaaactgtgtaaacaacataactatgtcccgaaacaaaga |
| | tcaataatttttatgggaatacaaaaatatccagcacttaaaaaaagtaaaattgacagtgtctggaattcaattgaagattg |
| | tcaggaacataaaactgcaggaatatgtgacccataataaagagaaaaattaatcaaaactgaccaagaagtgacaca |
| | gatgttcaaattagcagacagggaacttaaacagttgttataactgtattgtgtatgttcacaatgttaattagagatatatga |
| | aatataaaaaagacacagatcaagcttgcagaggtgaaaactacaatgtttcagatgacaaatacactggatgaaattag |
| | cggcagattaaatgttcaaaagaaaagataattgacttgaagacagcaatagaaatcattcaaattgcaattgacaaatg |
| | gagtttaaaactcatatagagtctttgctattgtgaatagtgccgcaataaacatacgtgtgcatgtgtctttatagcagcat |
| | gatttatattcctttgagtatatatccagtaatgggatggctgggttaaatggtatttctagttctagatacctgaggaatcgc |
| | cacactgtcttccacaatggttgaactagtttacagtcccaccaacagtgtaaaagtgttcttatttctccacatcctctccag |
| | cacctgttgtttcctgactttttaatgatcgccattctaactggtggggggggaggtgggaaggatagcattaggaggtat |
| | acctaatgtaaatgacgagttaatgggtgcagcacaccaacatggcacatgtatgcatatgtaacaaacctgcatgttgt |
| | gcacatgtaccctagaacttaaagtataataaaaaatatatatatatgaaacttagaaaaaaaactcatacagaactacaa |
| | gggacataaaacagccaaaacagctttgaaaaaaaaaaaaaagaacaaaaagttggggacctgcactatgttatttcaa |
| | gactcgtcataaaaggtacaggaattaagagagtgtggtactggtataaaaatgacaaataaactaaacagaatagagtt |
| | cagaaatagacaaactcacctatggacaactaataattgacaaaagttcaaaggcaatgcagtggagaatgaatattttg |
| | tttttatgcagttggaataattagatactcattttaaaaaagtgaatctcaacatatcactcatatgttatacaaaaattaactaa |
| | aatgggttatttacctaaatataaagcataaaactaacttaagaagaaaatttagaagaaaatactcatggtgttgacttgg |
| | caaaaatttcttagatccaaaagaatgatttaaacaaaaaagctaagaaataatactctggataaactgcactttaaatattt |
| | gagaagcatatatgtgatataggacttacatcccaaagagtcaaagaatgctcaaaactcaataatatgaaatcaaacaa |
| | cctaactaaaaaaaaaggaggggattaaagatttgaacaaccacttcataaaagaagctatacagatggaaaataagaa |
| | cagaaaaaggtgctcaacattagtaattaggaaaatgaaaatcaaaacagaaatgagatacttattagaatggctaaaatt |
| | aaaaagactggccataccaaatgttggcaaaaatattgagaaattataattctaactggcatgaatcagatttttactatata |
| | ttcgtgagtcttcacctttcagtatatggatttttttttctgtcttttaatacagtactgtgtttccgtctcttctcctcaacacattaa |
| | tgtcccattcactatacccctagctcacacagcctttaatattaataaaaatctcaagttggcatctaactttagataccttcct |
| | gatatcctgatgtgggctatagcaaacatttttttttttttttttttttgctacagaaatcccaaagaattgttaagcataggtatat |
| | ataaagtctcttgtcaacctttaaaatcaattaacttcagaacaaaatctcaaatgtcataattaggggtgaatcactaataat |
| | tggaaatatcagttttatttcttttctaaattaaataaaaaattaaattcctctcttaggttctatgagacaattccatagtaaacc |
| | ttatctctaatgtattttctatccgctagctgatttctttatatacagtggctaaaaagtttgttagactctaacttcagaaacaa |
| | gttcatatttgaatacttttttttctaatgacaatgcctatatataaaatgaaaaagcagttccaatctaccctctatagtataac |
| | actatttctacttagaaactaataataataatgataaatctttgcttttatcactcagtacctgagcttcttcctgttgctttttaag |
| | ctgttcgagcatctgctgaaattcggcctctttttgttctgcttcttccaaggtggcctgattctgctcctcataggccatggc |
| | caccacagccaggatcaaattcaccaaataaaatgagcccaagaaaatgaccaggacaaaaaatatcatgtatgttttc |
| | ccagcagcacgtaatgtctaggggaaatgggggataattccatcagtattttagtataatgtccccaaataaatatctgatt |
| | accacagttagatagtcagtagactaattagctttttagtatccagtttattttcaccaagaattttttggtattgatatgctcaa |
| | cacacaaaattaagaatcagattatttgaggcttgactgcagtcttgttggatataaatacattttaaactcatatatagtgga |
| | tatatatgtgagccccactataatgcttttattagagttcatatgcagagcaataatatgacaagtaagagcactgaaccttt |
| | aaatcctttcagtgggattccatttgcactgcagctattctccataccatctatagatgacttttgctcccctccccttctagtc |
| | tcagttaatagctagagctttaatttgaaattttcacatcaacatcactgatttttaaaatcttttaacgtgctgtttatataccaa |
| | caactggattatcaacttctaagagtcatgagcatctccaagttcctagcagaatgcctttcacataaaaaagtcctcagtg |
| | tacatatataggtactgactcagtgagcaattaggattcctggcacacttgctttatgttggaccttcctctgtgttccagagt |
| | gttataagcaggtcctttcccttcccttcccttccattcccttctctccctttccctttccctttccttttcccattccctttcccttt |
| | ccctttctttttttgagatggggtcttactctgtcatctaggctggagtgcaatgatatgatctcgactcactgcaacctccacc |
| | tcctgggcttgggtgatcctcccacctcagactcccttgtagctagggctacagatgggcgccaccatgctgggctaatt |
| | tttgtattttggtagagatggggttttgccatgttgttcaggctggtctcgaattcctggactcaagtgatccatccgcctctg |
| | cctcccaaagtgctagggttacaggcatgagccaccacacccagcctatgcttttcatttcatacatgtctataaaaataaa |
| | tgttatgcatgctcatttggaccttaccaactggtaaagattttcccagtagtcttgagtcatgagtcgaaatagagacagg |
| | aaagcccagctaaaggtgtcaaagcttgtgtagccatagttggggtttcgaccagccttcacacagatgtatccttctgga |
| | cactggctataagagagagaaatggaggtagggtcagtttagaaattacagcaatttcaatttatttgacttataggaatttt |
| | caaaacccaaagctgctatctgtgacgaattatcttgctttaatttccagaacactttaggtgattgcccaccgtgaaactta |
| | tcagatgaaattcactaggaagaccgatagaaaatttagatgcaaataaatatttacggacatttagaatggcagaaagct |
| | agcaaccacacacttttggctctgacattaccatatttctactctttacaaagtggcaagaatcaatagttaaaaattaaggc |
| | cgggtgcagtggctcacgcctgtaatcccagcactttgggaggccaaggcgggcggatcataaggtcaggagatcga |
| | gactatcctggctaacacagtgaaactccatctctactaaaaatacaaaaaaaaaacaattagctgggcgttgtggtggg |
| | tgcctgtagtcccagctactcgggaggctgaggcaggagaatggcgtgaacccgggaggtggagcttgcagtgagc |
| | cgatattgtgccattgcactccagcctgggtgacagagcgagactctgactcaaaaaaaaaaaaaaaaaaaaaaaaaa |
| | aaaaaaaaaaaaaaattaaaagccagaaaacagacaaagagtggggaatcaaccatttcattagctggttttgtagaatt |
| | ttctaaaataaagttaatatttataacaattttttatttacagaaaaagtatgaggatagtacaggcttcatacattccatgccat |
| | tttcaatattgatatattgttattatttaattgagatgggatcttgctacgctgcccaggctggtctgaaacttctgggctcaag |
| | caatctcctgccttggcctcctgaagtactgggattacaggtgcgagccaccgcacccagccttaatacattattattacct |
| | aaagttactattttattcagatttcatcagttttttttttttttttgttttgttttgagatgtagtctcgttctgtcgcccaagctgga |
| | gtgcagtggtgcaatctcggctcaatgcaagctccgcctcccaggttcacaccattctcctgcctcagcctcccaaatag |
| | ctgggactacaggtgcctgccatcacgcccagctaattttttgtatttttagtggagacagggtttcacagtgttagccagg |
| | atggtctccatctcctgacctcgtgatccgcctgcctcggcctcccaaagtgctgggattacaggcgagagccaccgtg |
| | cccagccctcagtttttttcttaatgtcctttttttgttccaggatcccattcaggatacacatcgcatttagtagttatgtcttttt |
| | aggctcctcttggctgtgacaatctctcagacttcccttgtttttgatgaccacaacagttttgaggattactggtcagatattt |
| | tatagaatacccttcaattgggatttgtctaatgtttgtctcatgattatatttaggttatggatttttgggtggaagactacaga |
| | ggtaaaatgtcactgtcatcacatcacatcatgccaagggtacatacttatcactcactatgacttatcactcttgatgttaac |
| | tttcaccacctgtcttgaggtagtgttaggtttctctactgtaaatttactcccttttcttccttttccatatgtgctctttgaaataa |
| | agtcactgtgtacagaccacatttaaggaacccgcagttatgatcttaagtatataccaaaattatttgaaattattctgcata |
| | ggatatttgtccattcatattttatttaattattcaatcatttcaattaacatgtagtcatggatagtcattttatattttggattacaa |
| | tttcaatgatatttcatttatttgttcaaatggcttcatctttggccactggaagctctttcagttgacacctgtaacagtttgacat |
| | acaccatggttgtgggttgcatttttttttgttttttaacactttcttagttttaggtactatcagatgctccaggctcatcttttata |
| | atttctgccgcagtcctagaatcaaccatttctccaaggagccctagttctttttattggaaaatggtattagaaaccaagat |
| | ctgagtgctagctgtgtttgttgctactggggtgtcattgcttctaggccctctcagctgacagaacaaggagatacacat |
| | gtttgtaattgctcagtactggtacacatgcacagtggtttcaggattgttaatctatactaccattggaaacaactttatcaa |
| | ctagggtattgttccttttacctttagtcttacagactccactcatttccaaagttacttaggttagcatgttattaccccaccac |
| | ctacagtgaggtcatttcatattttgcaatacatgtatatatttttgtcacatattccatttcttcctgggatccccaaacctcac |
| | aaaagattttttttttaatttgtatatattaagattcactctttgtgttataaaggtctatgagttttaacaaatgcattaagtttcata |
| | ttcaccattatagaatcttagagaacaggttctcaccctaaaaaaaaatcccctttgctttacctagataatccctcaccatg |
| | aacccctggaaaacactattttcttatcatctctgttgctttgtgttttctagaatgtcaggtaattgaaatcatacagtatgtag |
| | ctttttgaggttggctttttctcacttagaaatgtgcatttaatattcattaatatttgtttgtatcttgatagctcatttctttttaat |
| | cactcagtaatattttattgtatgggcataacaatttatttatctatacaactattgaaggacatcttggttgcttccagtttgggg |
| | tcattgaagggcatcttggttacttccagtttggggtcattatgaatacagctgctataaatattcacatgcaggttttttgtga |
| | agacataagttttcaaattgatcagctaaatacctagaagtgcagttgttggatcatatggtaagactatgtttattttgtaag |
| | aaaccgccaaactgtcttccaaggcggttgtctagttttgcattcccaccagcaataagcaagagttcctgttattctgcat |
| | cctcatcagaagttggtattgccagatacatttttcagtcattctaacagatgtgcagtggtaactccttgttgttttagtttgc |
| | aattccctaatgacaaatgatgtcgagtgcctttttacctccttaataaccacctgtactatatactcgtgtggggtatctgttc |
| | agatcttttgccagttttaaattgggtcatttgttttcttattgttgagttttaagagttctttagatattctggacactggtttgttgt |
| | tttgttttgttttgttttcttttgagacagggtataattctgttgcccaggctatagtgcagtggtctgatcatggctcactgcag |
| | cctcgacctcccaggctcaagtaatccttctgcctcagcctcctgagtagctggtaccacagatacactacaccatgcct |
| | ggctaatttttttatttttattttttggagaaacagtgtctccctatgctgcccaggccagtctcaaactcttgggctcaaatgat |
| | cttcccctctcagcttcccaaagtgctggattacaggaatgagctacttcacctagccccagtctttttttttttttttttaaatca |
| | gatatgtgctttgcaaatattgtcttccagttgctggtttgtcttttcattctcttaagaatatctttgtaaaacagcagcttttaat |
| | tttaataaaatacaaatcatttatttctttaatggaatgtgccgcttaaatacaaaatggttttgtatttaaaactcatcacctgg |
| | ctcactcctgtaatcccagcactttgggaggccgaggcgggcggatcacgaggtcaggagattgagaccatcctggct |
| | aacatggtgaaacctggtatctattaagaatacaaaattagccgggcgtggtggcgggcgcctgtggtcccagctactc |
| | gggaggctgaggcaggagaatagcttgaacccgggaggcggaacttgcagtgagccgagatcgcgccactgcact |
| | ccagcctgggcgacagagctaaactccgtctcaaaaaaaaacaaaaacaaaaacaaaaatctcatcaccaatggcca |
| | ggcgcagtggctcatgcctgtaatccccagcactttgggaggccgaggcaggtgaatcatttgagatcaggctcccact |
| | gattctacattatggtgacttgtacaattatttcattatatattacagtatgctaataatataaataatgtacacaataaatgtaa |
| | cgtgcttgaatcatcctgaaatcatttcccaccaccacccttggtcggtggaaaaattgtcttccacaaaacagtccctggt |
| | gccaaaaaggctggggactgttgatgtaaattacttaagccataataggaattcattaaggggaatagattattatggttta |
| | ataggaataaagtatgtattaaccaaatttatccaaaattttatctgggcttttatgttaatcatcatcataataaataatagtta |
| | ataggcaataaattcttttctttttttagagtgtgctttttttttaaatttattaaaaaaattttttggggggacagggtctcgctttg |
| | tcatccaggctggagtgtagtggtgccatctcagctcactgctacctccatctttcacgtttaagagattattgtgcctcagc |
| | ctccagagtagctgagatacaggtgtgccccaccacacccagctaatttttgcattttgtagtagagacaaggtttcgcca |
| | tgttggccaggctggtctagatctcctggcctcaagtaatccccctgcctctgcttcccaaagtgctgggattacaggtgt |
| | gagccaccgctcccagccatttttcagagacagcctatcacctaggctggagtgcagtggcatgatcatagctcactgt |
| | ggcttggaattcctaggctcaagcaatccttctactttagccccctgagaagttaggactacacgtgtgcatcacaatgcc |
| | tggctaagtttttctttttttcttcttcttttcttttcttttttttctttctttctttttttttttttttttttttttttgttttgtag |
| | aggtagggtcttgctttgttgcttaggctggtacatacaataaattcttacaattgtccatgtgatagttcaaatactttatgtgtatt |
| | aaatcattaatcatcacaactattaaagggacagaatattactctctccattttaacgatgagtaaactaaggtacagagagcttagga |
| | cacttttccaaggtcaaagagcttacaagtggtgaagttaggatttgaaccctggtatacatgaactctacaacctatgctctta |
| | ccccctacattcataataaggctaggaaaactgtcaggaaaaaaaattccccaaagctctatttatatgactagtatacattt |
| | tcattaactctcttagatttttatggctacaccagtttccctttttaaaggatgactcaaaatatatttaagttccgtgtcctaaga |
| | gtcacttactattgctatctctcagctcttaaaaaattccctattttgatttaaaattaacttacaatactcttaggaaaatatttaa |
| | gggttagagaataacagcatgactaagatattatggcatctttttgttgtaacgtgtggcctaaaagaactattcattatgtca |
| | actgatacttggacatgcctttggcacacatgcacccttggcttctttgtatacagaaatacatgctccttcaagaatactta |
| | aatatttcaaagcacttccccaagtgtgcttccaccctgaagaccacattatagtctgggtcttttccagtcacgacaaaac |
| | tcatcccattgatacatatgtacaccaataaaaaggggcccatcttgtgttcctgtgtttcctaacagctctgatagatgtac |
| | agcaaagacagactctgggccttcacacattggagcatcaatttaagaacatcttaactgtgaaattttggggaaaggtct |
| | tcacaaagggataatttttaattgctgtgagtttaggaccagtggcaatccggtaaggcagacaaggcacttcctagggg |
| | agcagcactgctctttattgcatacatggtaaggctacacatataaccatgtagttgttttcctacctacttttttttttcgcaaa |
| | gagttctatatcttaaaaaatatattatgtttcttaccctgcatctgagccatttccacagagtaaagggtctttttgcccatcc |
| | aaaacataaaagtgacctgttaatacaaaaaaaaacccattttatttcatattaatcctattcacattagtattagtaataaatc |
| | agagttggactatttcagttatttacaaaggtggctgtacacccacagtctcaactatttatagttgaaaattcattcagcaac |
| | actaaggttaacataatgtaatacttcttactgtcatctccaatgtaatccttccagttaaatgtgctcattgttacattaacaaa |
| | tgtcccatttgaatccattgtgccattaaagtaggaagtggtgttggtttcaaaagcagaatcgcttgggggccactgcaa |
| | acatttattcctcagattgcccatgaacagctgcagcccaatgagagcaaacacgctcagacagaacacagtcaggatc |
| | atcacatcagaaagcttctttaccgactggatcagggcccccacaatggtctttaaacctgcagagagagaactataggt |
| | tacctgaggaagagtgccagaaatcatgatttcttaatagtcacacacattctctttcccccataaatgattgcttgactattt |
| | agacaccaaagctgtatggatagcctgatgatttgggaaatagatgaacagcagattttacacatgaacagtgtgcttgct |
| | ggaaattcaatatgaaggtttaaatatctagtcaggaagttttgtttgtgtggttttacatgctgaacaaacatacaaaaaaa |
| | cttggagttatttttcatttcagattttgtgtttcatgcagccattttccaccaatccattctttactctagcctccaagcagtagt |
| | aatttagaggagaaaagaattttaaaataatgtaagtctcatgtctcatgccatatacatgctcttcaacaataatattttctcc |
| | ttgtaaaaggagatccatttttgaactacatctgcagcgaatggtaaaatccaaatctgttagtaaagccatggtagtgaa |
| | aaagagagagagagagcgcaagaggcccaaattagcattttttgaagatgtctcatgcaattttctgttaaactcaaagg |
| | ctgactttataaatacgcatgtaatttataaaattttaggagctaaactgacattgaaacatcatttggcattatttaacgggat |
| | gaactgtaataataagcaacaaggctaatgctgtaagtcatataaattgatttcaaactcaataattaaagtcaacctcggt |
| | gtttaacctagctctcacctggaatgactgaaattgttttcagtgctcggagaactctgaatgttctcaacgctgagacattg |
| | cccaggtccacaaactctgtcacatatctgtaataggggagttcacacacaaacacaataacacacaagaaaagttgga |
| | gatataaggggcctactaccttacaccagtttcttcttacctggaattacagaaatagttttcagagctctcaagactctgaa |
| | agttcgaagggctgaaacattgcctaggcttacaaattctgttacatacctgcagaattaaatcagagttactgatagttttg |
| | gcaaagtttatactaaataaggacttaatgctgggtttggcacatagagccctgtgagtttaacaaatggaattgctataga |
| | cctcaggctgccatatgctaaaggttgcttttaagaaacaaaatcgtgttgctttatttcaatgtaattttcactaatatgaaca |
| | catttgtttgcaaaactaatccaagtacagttgttgctattaaattgtagtcaatgaataacttcattttgatgaagagaaaga |
| | ctatatgacatgaagataaccccatttattgatcatttactggatatatttacattgcaatatgtattctaatatgtattcttaaaat |
| | acacaatccatatagttaacttgactaacaagaaagctctacatttaatatatgacacaaagaccttgtttgtactatgactat |
| | tttcactcctttgcgcttatcaaattttcaaagttactcacgccatcacaatgacactgaaatccagccagttccatggatca |
| | cgaagaaacgtaaaatcttctaagcaaaaccctcttgccaagatttttataagtgactcaaaggtatagattccagtgaatg |
| | tgtacctaggaaaaacatccaagccaaaattaacaattttgcttgaactgttaaaataaatgttttcaatcatagcaaatcct |
| | atccttttgtggtttttcaattcattaaaatattgttccttacttctatttaccaaataatctattcatttagttagttttacaagcct |
| | gagcaaagagttaaactgaagagaaaatatattctttaggaatctaatctggtttaactatttgggttttagaataatagtttcat |
| | gtatcttatggataatcatctagtttgaccttctagcgcagcagtatctgatagaaatgtaatggaagccacaaatgtaatttt |
| | caaattcctagtagcaagcgaaaagatgagaagaagcaagtcaacgtattttttatactgcattttatttaaaccaatatatc |
| | taaaatatgaaaattcaacaattaatattagccaaatttcaaggttccatagccatatgtggctagtggcttcctcatcagac |
| | agtacaactttagaggcaacatagcttggattcatttacttaaataataaaatattttattcaaatttcttaatgagtagactcat |
| | tttgagggctagtttacatgtctaccatagaaaatatatttcttttttttcaatttttaagaatcatgtagagttataaactgcccc |
| | ccatttctctctcttctttctctctctctgccctccttcctcccaccctgagtgtatgtgtgtgtgtgtgcatgagcatgctgtat |
| | agagatgtgtgtgtgtgtgtatatattatcatatgaaatgttccagttcactagtattttatatgtgtgtgtttgcacaaatgcca |
| | aaatacacggatacacaaaattcttaaatcaaaccttgcatcagctactatctctatgcaattgggctaaatcattcaaacct |
| | tgcttaaattcaattttcagcctccaaaatggtggaagacttttctatttgctgtttaatgtcatgtatttctagccctcttttctttg |
| | agtcttgacaatataagttattgctgttttcgtttttttcccaaaatagttgagagtacttgtttttcaacattgtgttctttaaaag |
| | cacaatttaagtttatttttcagagcacttcacccaagccacaaagatttgatttactcatattaaattcattagatttttcagcct |
| | accagtatttatatatgaaaaatgttaggatcattattttgaaatgttttctgcacaagtttctttcatatgagggaacaaagac |
| | atcaagaataatttatgcagatatcttcaagtctagttttcttaagtacattcccttccttttctcccccaacaattcaattgatct |
| | cataagcaataattcctatctttacttatatgaagtagctgactattaggaagagatatattttacaatatcaagcttatgtttac |
| | tgtgttgtagtttggatcgttgtgtgacacacataatcaccgtgatatttctgtctgtagagatagctggccttttaaaggcct |
| | agaaacaccacacatgtatgttctgtttcttgtattagtggttagtgccctgtggtttatgctgaggcaattctgtcttatctata |
| | gagttactaggaataagattaaagtccctacttttgttcttgatttggggaaattaagaaaatatgtgggtacatttggaatgt |
| | atgggaagtgttcatggctgtccatggaagagagtcctagagcctactgagacagagctctacacagaataggtattca |
| | aaaatggttattagtaactaggttgggcaggtaagtgggagtcaggggacctttatctttcgtggctatgcccacactagg |
| | ctcataatcaagctgtcaattctgtctatgttcctgtaactttggtgttttaaaaccttgaaatttggctaatcttaattgttgaatt |
| | ttcatattttaagtatattggtttaaataaaatgcagtataaataaaattaaataaaatgcagtctatagaagtgccttgccttat |
| | ataacattcccctcactatataatatggttttgtgtatgccacatctttgtacagggactgtttcaaatatcggtgacttcagttt |
| | tcatctcagaactaagttatcttctgtttcactgaacccagggtttggggtagtaaccactactactttgcttttttcctgctgg |
| | cttgctttcaagctactgaagacaattgatcagaaaacctgagcaaagttttccagctgaatgatgcaaatcaaaaactttc |
| | taaagctaagactgccttcataatgggtataatcatctctaagttattactgcttctttagaaagagaggcctgtatattcaatt |
| | gccttttttttttgttttgaaacagagtttcgctctgtcgtccaggctggaatgcagtggtgggatcttggctcactgcaacct |
| | ccacctcctgggttcaagcgattctcttgtctcagcctcctgagtagctgggactacaggtgcttgccaccacacctggct |
| | cattgtttgtatttttagtagagatggggtttcaccatgttggccaggctggtcacaaactccaactgccttctttaactatctt |
| | taataatgttatacaaataaatgaaattgatacatgttttaaatgcttcaataatttctgctgggaaaaccacatacagagtag |
| | tcttacataggagtgtccattttgttaactccaaaatcatgtatagtaaatgagagttctaagttaaatggctttcctcaatatg |
| | ggagctggattctctattacaataatttctaggcagcattttgaagctactatttttttttaaataacatgttaaataactgacaa |
| | ataatggcttagagtggtacagtaaatcctcagagaaatctataaaaacggaactaaaagtggcagtatttgttacactat |
| | cttatctccgattgtacattacttttaaaaatataacaataaaaagagcatactgcattttttacttaaccagtcagatacctga |
| | ttattgtattatactatatataagtagcctatataaccactttgtttcactaaaaactatgtttctttgatgcaaataacctcatatg |
| | tgactgaaaaataagaaaatgaaataatgttgttacgataaagaaattgtgttagttcatgcacagttttccccagcatattg |
| | atgttttgaaacaatgaagggcattttttctcacacttaaatggaaaatcccttgcaataataataataaaaagatcttgatac |
| | taatacacaacatccacagaagtgttttcttttagtgcaacaagggcctggtttaacatcctgaacaactaatgtgcttaga |
| | cctttctccatgtattaaaatacagaatgaagcattttattttgatgaaatcatacaaatatccccagtctccatttcaaggga |
| | gaaaggatgaggcctgggatatgaggccacaaagaactcaatggctatgattttaggtacaaacattagaaaagaatca |
| | actgggcaagtgtctttttattgggattattatggtttctatgttgtattctagttacaaagttccataagttttactctcccccaat |
| | catgtttcccctaagccctgttattttatgaacacagttttatggaagacattttatttcttttacagaacacatctaagctgtttc |
| | agcacacatgttgtgtataaaaaacttacatatctaaatataacttagacatcttagatgtataattaagacatctacttaattt |
| | agagctaaaaacctaaatctattctattcgaatataacaataatcactatatactggaataaaataatttaattcatcaaattac |
| | tgaactgatcactgtattttatctcaaatgattaaccatgattgagacctaattttaggctaggtgattggtttagtttaccaga |
| | aagcatatactaatttataacctctctttaacagatattgtcatgattttgttggaaaaatacttacctcgtagaagaatagctc |
| | ccccaaagaacttcccttatcttctttcatattatgaaaataactttttcttagacagaagtggaaaccataagtcaggctata |
| | cccacaaggagattgctagagaatttgagtgtgcatttctcattcccagaagttattcctacttactctacattctttgtccagt |
| | caggagggttgctcaaggtcataaatacacagttggtcaaaatagtgcacatgataagcatgctgaataaagtagattat |
| | agttaaggaataaatgttagtaggttaccatggccatttataaatgatccaaaacacacaaaaaagtttatcgatgcaaaa |
| | actgtgaaattaatattatttgttttaaaacaatgataaaataataagaaacttcaaagcctgtgatatggccttggtgcactc |
| | tttttttcattttataccatatactcacaaaactgaatttaatagaaaaatgatctctatttgaagcaaatgcaagttattttgcatt |
| | ccaatcagttgtgtgaaattgccctggaagaagaactcaccatcccttagtaagtttatagtatgccataagtgtaaaaggt |
| | gtacttcagatatattcatcctcactgacttataaaacgttaacgaatgcaatgtaaaaagtgtgagaatgtagagcccagt |
| | ataatcaaagattacattgattgagaattctatcataaatagtaggtttgtagtgatgtaacttactcctttacaatttatagcat |
| | aaaagagggagatccaattttgtgctgataaggtaatgttattttctagctttttatttacactttagattgtggcggaccccaa |
| | cagaccatctggctatactcttgtatctagtccacattatacctaaacagatagcactattctgatgctcttctgagaattagc |
| | tgtagtacgttctcttgggatctaagtccagacttccctgtaagaaagtttatgattattttgagataaatattttattttagaata |
| | taaatactttccaacatcaagccatagaatgagagaattcatacctcaaaaggttatcagtttcatttgccctgtctctaggt |
| | gaatcagtctttaagtttatatagaagcctgctttgaatctgcctatacaaaattcccattcttgaacactgcctcagaaaatc |
| | accattctattttatcattacttgagaattttcttgaaatccaaacatattctgcccttttacatatgctttttgactaacattgtaaa |
| | taatttcaagttcgactttttgatacatattctcaaccaggtacatgagcacatagtaagagtctgataaatatttactgaaaa |
| | aaagaagggaataagacggaagggaaaagggggaaaaaagggaagaatgaaggaagggaagaagacaggaaggacagaaagaa |
| | ggaaggaaggacacaaagaaggataaatccaggcgctgaagatacagaaacaattaaggcgtaaaagtttgaacact |
| | tggtaatgcaaatagtaatactaaccataatctaatctttagcaatggttatatatgacctcattgaagctgataaaggtgtaa |
| | ggagatttgctctttttcctactatgtgtttcctttgcacattcactatcatggttggttacatattttcttctataatgctgtagtca |
| | ttctataatactctcattataaatagtggaaaaccaatgcaaataaatgaattaaactaacaatgttaccattaatctcaaaga |
| | aattttacagatacttgaaaatataactgacattttctttaaaatcaactgttagaaatagcatttaggcaattcacattaaaag |
| | gatatgaatgtaccaaaatcttgatagcaattttcctaacagggtttagtggagttaaaatatacaaggcagaggtggcac |
| | tgaatcggaaaattgcctttcctttattcattactataaaagtctgaaaaagaaaatacgagtaaaattactaaattagacatg |
| | ggcttatttaaagagcttacatacatgaagaactttttaaaaaatagaatttgtttgttcaaacttgtttgtttaaaccagttgat |
| | atatcataactacaaacatgttagatgtttaaattaacatatagttacaaataaagatactatttactattacctaatctatctcta |
| | aggttccaacttgctgatacaaactgacaattctacacaaatttttttatggaattgtagcgttacagattccaactctatttaa |
| | agcttaccatctgtcttcagctttggtaacttttcagattactctgtacacattgctactaaaataatcttttataagtattttaatc |
| | atatcaaatcttttaacttcaaagtttatgatggcttctgattgtccctgtagatttaaaatgttcaaacaggttgaataatctgg |
| | ttttgctgactatagatgtgtgagaagggttcaacttctcagctaatactgaagagagatggatgcattgaaatttagtctga |
| | aatacaccatttcaaagggcattccacaattaaaattctatgatctgaaagctacaatcatccacttagtattagctatgcta |
| | caagtgattatttaccatgttcctatagaatcatcttttatttttttcccactagagtcatctttgctatggccaattcaattcagtt |
| | gtcagcctcactcacaactagtcaaactctccctatgactctgaatatgaatgtcatagactcctagaagaaggtggcaac |
| | tgaaaggtcatttaatgcaaactcccttcatttcagggaaatgtctccacaacacattacaatatagttccacctacagtga |
| | ccccctattatgtcagaggttcttaatcctgcctgaatattaggctgtcctgtaaagattttaaaattgccaattgaatgaaaa |
| | tttctgagggatgggcacagatttgggtatttttaaaactttcctgaggcgattccaatatgcaatcaaggttgaaaatccct |
| | tctgtctgggaattttctcagctatcaatcacctccttaatcatgtccttccaaataaatcacactacctcctgatcactcccta |
| | attgtttctctttaccagtaagtcttagtacattaatttgtactcttttacatgttgcttgtaattatagtcaagtagcaatttacac |
| | agtgttcattccagtgagaactaggttagaaattgttataaaccattgagcataaagagcttgtaatctactttttctccattttt |
| | ttcacagtatttaatagcctggtttgcaaataaacacttgttttaattaaatgtgggtttaccttaaagatactatttacctttaag |
| | acatctattctgtatatccatttacttaataatatgtttctctcctttaactaggggctatatgccatacatttatgttcactgacaa |
| | catgctttgtttgagtaggtagacaaaaatatttatattgtcagtataaagggcagggaaaacaatttctctgttcattgctctt |
| | ttgtatttaagaccatggtgccaaccattacttaactaaccattttccagaaactgaacagtttttctgttcattatccctttgtgt |
| | ttaagaccatcaagccaaccattatttaactaaccatttttcagaaactacacagtttctctgttcattgcctttttgtatttaaga |
| | ccatagagccaaccattacttaactaacaattttccagaaactgcacactataaatgtgaatagttttatgaagcgggcaa |
| | gcaaactattctttactagcagtaggactgtgtgtaaaggacagtggaccttggaaagactgttgttgaagcacagaagc |
| | attaaagggttgccttggagtcacaagattagattatttaatattacagttgaacagtcaagatccatttaggcattcagcaa |
| | cacaacagcataacttgaaaaagctcaccgtaatttaaggttcaggtactcatgctcttttgaaacaatttccctgaaaaag |
| | caatttctgcttgcgacattaggagagagtttcattttgaactatctccaaaaacagagtgaacagcaataaattatcccttt |
| | gaacatggaaccctttgctcctcctgcaagattagccttcgtagtactagcggaattacagctgacatgctaagagcatct |
| | tcgttgaatttataggttctaaaacagagcaggccttaagactacaatcccagtttatgtctcaggaacaatacctgacaca |
| | aagagaaccctgagttctgagaaatgcagaataaaatactaatgtcctgcatagtgaaagaattgcaagaatttgaaatta |
| | tggggctctgatgatatgaagagatatacctaaactgaaaattacttgagaaaatgacagatgaataggggagagatag |
| | atcgtcattgctacagctggaagtatgttaaaccagctattttgtactctggaatgttacttgcttctttttcctagaatgttactt |
| | agatgcacatcttataagtttactaaaagttttactactttgtggcaagaataagatttacatctcaatgattaggaccacatc |
| | aatcagagaactcaaaaaatgatgcagccaattttttaactttcccttttattttattatgttaataagcatagtatataatttcac |
| | tagtggttaagagctctggctctggagtcgaatgcctggactcagatcctggattcaccacttaaggagtgtggccttga |
| | gcaagttacctcatctctctgtgcttcacttccccatttacaaactgagctaacaataggacctgcctcactggttcactatat |
| | agtaaatgagttcaagaacttagcagtgcttgacacagtatgagcacaagaaaagttagttatgattatcactgtcactatt |
| | attaatagcaccctggtcaaattaaatgccattaaaatacattgtgatattactcatttaattaaattaggtattcaaaatgtatc |
| | actaattaatgatggtaaagagttacataatgctttaaagcttttaaatgccttttacttgtatttttccatataattaacacagct |
| | tcataacatatttttaaattctaaaacttcctccattagcttcttactttactccttctatgctaagaatgacaaattacctccttgt |
| | ttaatagcacctcaatcttcatctttatggatacttcataaacgaaagcagtgcagccaaccgtaaagactatctagatgtct |
| | cgcaagatggttcacagccactatgaataaatcagggtctaagtctggctctcagccatggaggcaataaaaattgactt |
| | tgtcaaaaacagaggacaattctgttttttagtggcagactcacattaaccaaacttgaaaacctgatatataaaataagaa |
| | tgccaacctatttatttatttagacaggacctcgatctgtcacctaggctagagtgcagtagcacaatcatagctcactgca |
| | gcctcaaactcctgacctcaagcaatcctcctgccttggcctctcgaagccctaggattacaggtgtgggccatcatgcc |
| | tggcctccagtgtatttatttacaatgagatacattactttcatatggactgagataattgtagtgatgtaatgagcacgttctt |
| | ttcttcaagcaggtttaggggaaaacaaattgtagtcagaaggtttcttgctgaatatggagcccaagccagctgacctaa |
| | tcaaatgatgtggaatgtttaatagcatgtgttaggtagttcagtgtagcccctccacaatgaggtggcctggccattggg |
| | aagaaaatgctttcagtgctactactaaagttctgtctcccttggaaaatagtaacgtttgcctctgttgttctccttttgtttcc |
| | cccttaatcttaccagcctgattgattctcttttttaaggacacttcctttctttaagtagcaaatacttttaatcatagcagagta |
| | aatcatgcctcctttcttaaagatatctttacaaacatatccttcagagcaacagttttcaccggataatattttgtccaatttctt |
| | ggggggtgacttattgtaatcatcataactacatttcaaatttttctttatcaattctatagtaaattgttgaaataagttattttta |
| | gaagtacatgaatgtattaaatgtcaccttttatgaaactagctataaaaggggaaaaaggagactctgtttagctatttaca |
| | tgtagtcttatgactttaaataaaacattaaagcttgctattcaaagtgtggccatgtaacagcctgatgatttgtaagaaatt |
| | caggatctctggccctagcccagacctatggattcagaatctgcaaattaaaacatcttgaggtaatctgtatggatgtgc |
| | aagtttgagaagtacttatttagaacaacaggactaattccatgtctgaatggttgtcattgcctttagccatcattgattttat |
| | aaattataaacttttaaaaggatgtgctttgactaataaaggtgtttaatatcttccataatctataagtcatcaactcatggtg |
| | atactaatttatctttagtttctacattgagctatttttacgcttttaagtgtaaaatataaatcgtggttataaaatgccttaatatc |
| | caattagaaagcatagagtataccacctattttttatattttctataaaatatcacccagctagtttaactatagaaaccagcta |
| | attctgatacaattatgcatttattttagcattaggaaagcacagtcataaccactaatttaaatcatgtatggatggagaaaa |
| | tttgaaagttctttctttttggtcttccaattttgacatttttttcttcagggtgatgttttaatggtatacatgatccagaatctagc |
| | atgccacaaactatataaggttaagagaagatcctaatcatattctaggttactgggggggaaaggaatctctgggtgaa |
| | catgcttcttcatgtattgtctggctacaggaggatgctaagcagattatttattgtcctctagaatataaattattgtgaaggt |
| | acatcaagtcagtcatgaactaaaaaataattaagtacaacatatgctcataattcttttctgcacaggtattattctgtagtct |
| | accctgtgcttgaatatgtcagccataacagatatttcactttgttgatattaattttaattttgactatctgaattatcactgtga |
| | aattgtagaaatcattcatctattcatatttcctgactgtaaccaactcttgacttcatttcttataaatgtttgttttattatattgtg |
| | ataaaaataagtgacagtgaagagtattataaaatctttaacagaagaaaaattgtgatgctgtatataaggcccagaaaa |
| | gtatattacagttaagagtttcattaaagatttattagaagtctaaaatcaatactcactttcttattgatatagtagggatccag |
| | gtcctccaggggctctgacaccatctctggaggaatgtctccataaataaatggaaggttctttccagcttccaagtcact |
| | atttggctttggtttgttctcatcatcattatcttgttcctttttgggcttcttggctttctcttctgcagcacgtttttcgatagcag |
| | caagagattctctagtaaaaaggcggaagctttcaggtcctgggggtaccaacagtgcctgtgccatcttttcatcctgca |
| | catttaattacgtgtagcttcttgcatacgaattacctgcaataaaagaaaaattgcacaagagttaggaaagcaagcgatt |
| | gggcataagaacatcaaccaaaatgtcattttttagtaactttttaaagctataaacattaagtcatgcttttagtactgcacta |
| | cacatttgaacttgaaaacatcatcattcaacctcattctcaataagccaagttcataaatctatcaaaagtctgtacttaatg |
| | ttaattagtttgatttagccattctacaatgtacagtatatataatatataaacattatattttacctcaagaatatctataattttgt |
| | ccattttttaaaagcatttaaaaaaattctgttttgacactgtaagccaaaatatagtatactttcagcaaaagagtaatgcca |
| | gtaaagtttgtcaataaaattgcttaattattctgcatcctgccccccgccttttcataactttgtaactccccaaataaagaa |
| | gtactggtaacaatacaattattgtaatacatccctgactcatacaaatgaggatagaaaatgaagttttgccaaacccaac |
| | cactgttctcgttgtcagcatcattgacaactgctgctccagaaaagatctgatcaccaaccagcagggacatggaagc |
| | cactgctgccagaaaaatctacctatctttagtatttcctttcttttattaagccagactctgtttccttctgaggcaatggaga |
| | atatgtttactccttatttcaatatgctgcccttcaattattccaagacaattttgacgttttccctgaatcttccatttagtatagt |
| | ggttaaaagcatggactcaggtgctacactgtccgttctaaaccctggctctgccgcttatgagctgtgtgattgtggaca |
| | aagtaaccccttattgcttgaattcatctataaaatgagggtagtagggaaactgacttcctgggttatcataaggaataaa |
| | tgagttgattatgttgtgtctagaatagtttttggaattagtaagcactatattattgtaaactatcataattaaattatcttcagtt |
| | tccctagctattttccctcttttatggtttcaggcatcctcacaattcaaaccactctttaaacaagttcttgctgtgctggaac |
| | cacaattgtgactacggaattaaaggaatgaactgagaagtgcagtgtatagaggcacactcacacatgccgatcacct |
| | ggatgagaaagaagggaaggcagactgatggagtggccctgctcaatttctcagggaagggaacaaaaatctatttgc |
| | aggatacataatagaactatcaaaaccacaagaaatcagctatgtaaatttcgaaatatgagttattttattttgcattatgcc |
| | aaaaatcacatttaaaaaatcaagatagatagaatatagaaacaatgacaaaccaaataaaataagtacatcagtcatctt |
| | ataaaagaacctaccactatggcaccaaagtcatgatcaaagtcatctgtgacattttccccattcctaccattcccctttgc |
| | ccccacatgtattctaggctccgtagtagtttatccctaaatggccctctgattcttggcagctcactacatatatttgagctt |
| | acaatcagaagctctggatctattaataacataagaaaaacaatatagcttattatgtgtgtgggggtttttttgcttatatctt |
| | aattactagttccctgatctattccaatgacaaaaaagtgtttctataacattacttattatattataatattattatttttttgagac |
| | agggtctcactctgtcacccaggctggagtgcagtggcacaatcacggctcactgcagccttgaactcccgggctcag |
| | gtgatcctcccacctcagcctcctgagtagctaggactacaagtgccctccaccatgcctggatcatttttgtatctttgtag |
| | agatgaggtttcagtcgccattttgcccaggctgatctcaaacgcttgggctcaagccatccgcccgcctcggcctccca |
| | aagtgctaggattacagcggtgagccaatgcactgggcccaaaaagtgtttttaacgtaagatatgtcttttctttgagtta |
| | gtatttatgatataacattgtcattatatcttttgctactatgaaggtatattctatataagttataaaataaaatagcaggcctgt |
| | caaaagttatgttatttttctctactaattttgaacagaacttctgttttatagttgggcagcacatttttttttctccatcaggttgt |
| | atagtacatgaatcgaaaggtttgagaaaaattctgttttactcaaaagtcagttgatcatttattttgtgttttctaaatttacg |
| | ggaggcagcacaaaataaaactaatgtttttagccatcttatgtcaattttgatgatcacatcatcaatccaactctaagctg |
| | tgtggaataatcacagcttttatgcttttgagtgcagaaagagtttgagcattttctgcaagccgtaagtacctaaagatgtg |
| | taatccatctgtctgatatcacctgccatgaactgaatggctgtggctcttctccagtgctgaatgaaaatgaaacagcatg |
| | acaaacatgtcttaaggaaattctatgaaacacggtcgctttcttagaggagaaaaaaaaaaaggttgatttgaggatttg |
| | gttttgaactaattttgatgtctttattgacaatgaatgaaaaacacactgcaattgaaatgggagatctgggttagtattgag |
| | tctgctgctaatggtatgcctgtatcatctaacctgtctcagcctcagtatcattgaggcacctcaacacagaaatggctgc |
| | ccttttccagatgggaatagctaatttatattaatacatacattttacttaattggattctcctggttggtttttgttttagaaataa |
| | cattaggtttaaaatatctaatgtggaaatcaccgagtaattttcacaaattattctgatttatctgagatgatatggaacaag |
| | gtgccatcacaaaatcatgaaaaactttttacaaataaatagtcctttaccttcttatttacttatcaagggctggaataaaga |
| | atttgaatcttggtcaaaagaaagtaaaaacgtctcgttgaaattcccatactgttgtctctaagtcctcggaagataggcta |
| | gttccagactctcagcttttcccctgcaagagaattagggtttgcccaatatttgggtaggctgagaaattaaagaatttagt |
| | gagtagagtttctcatggcttcaatgacaattttgctgtttggaaatccctcatagttctttcgcagttctcttttacttttttcctc |
| | tcctttcttgtactctgtgagcttattatacagagttcttacataattttatattcacattagagcagactttaaaaattaattctta |
| | ctgatgccttggtgtggaatatatatattattaatgattatatgcacattataatttattaaaatacataaaatttaatatatcaaat |
| | tttctacagagctatctttgcaaggacaaaaaagatacgaaaaaaatcttaggagatgatctctttaactaaaaaccaaaa |
| | agcctgtggatgaatgtgtgtaggtaaagcaaatacatccctttgttaactagtaaaaagattatggttcagtatatcatattt |
| | gaggaataaactgcggaactccaaatctggaaaggatctcgaaatcatttatttggaaaatatttatggagggattctacg |
| | aatcagtactctgtttgaaccaggaacccatggtcacttccctggggctgagagtccaatgtgggaggcagatttttatac |
| | agataattataacacagttatatatggctggttattgtctgaagttcataaaaagtatcaatatgttcatcccagggaaggga |
| | gagagaggtatggtttggagaaatttccctgaggaagtgatattggagtaggttttgtaggctagttggaatttgattggg |
| | gtcttatggtggtagtggttgggaaaggattggtaggaagacacatcagtagagacacattgagcaggtggggtgggg |
| | gtggcatgtgtgcagatgcagagacaagaaagagcatggagagtttgggagcaatgacaagcaattcattatgggtgg |
| | acatcaccctgcacagaagggagtggtatgacataaaactctaaagttaggcaggatagatcactgaggaccctgtact |
| | acacacgaaagtatgccatggatggggagtgttatgatcaatgtgaagattggaaagatggtggcagtatgaagcgtg |
| | gacaggcagggagtgatcctagagcagtagttttcaaatgttgtggtttcaggatttctttacactcttaaaatcattcatga |
| | acccaaagaacttttgtttatcgggggacttagctatccatacttaccatattaaaaattaaaatgaaaaaatgtaaaaattct |
| | tattaactaataataagcacttgtatgtttatacagacaatagatggttaatgaaaacaactatattattcaaaacagaaaaa |
| | aatttaatgagaagaatttgtttcacatttagtcaaatatcttcatgtcacttaagagaatacatttagattttcataaatgcttct |
| | gtgttcagtctgatagaatatgttgttttattgaaatatatgaagagaattttcacgcatatgtgtggctggaaaagggagtgt |
| | tttaattgcctttccaggtaactgtgaatatccttctttaatatcacgtcaaaactctacaaatggtagtttattaaattagttgtg |
| | tgaaatcaaaagccatatcaatgaacttttgatttatcttgtcctttgaataagtcttttacctaatcatgcactaattatttggaa |
| | aatggcacattgttatgaaggtcttccagattttgacaaattttattatataatatccaaaaatcgcatttgttactatcaccact |
| | aatgtcatgagaaaagttgtaatgtatgggtaggctgtcaagctcccagtggcaggtaaaagttttcccaaattccaatttc |
| | ctcttgaaagctcgaattttgacattgacaacaaatgctgtcatttgcttttcctgaagtgacaggctttccctttgttcattttt |
| | gagaaaatgtgtgccgaatactcaagtctgaataaccatagtttgtcagttattctttcaagtgaaagtggtgttccatgaaa |
| | aaaagaaaagtggcttattcagctcctgactcaatcacacaagtgcttttcctagagacaactaggacacttcagtatgca |
| | gtcaaatactttatatattcctcctatttcatcatacagaatgttaaaaatatgtatttttaggattgtgcttgaatgaaattaatat |
| | gtactgcttcatcagagatgtattatgtgaactggcattatttttcttactgagagtgtgcagcaatgaagaaaatgcagact |
| | atgcctatagtttgatgctactgcattgattcctgctaaagcattagcagttttagcacatgcctattgctttatcagtgcaagt |
| | gccaaaacagtgaaaaaagacaaatgccattttggtattattacgaaagtagtttagagctcaaagctcccacacaaagg |
| | gccccaggaatccccagttgtctaaaatctatacttcgagaacaactgccctagaagaaagcagttagtcaccactcaat |
| | agtcaaaagaagagaagtaaaagatctgactatagctctggtaccaaaaggcatggacttagaaatcagaactggattc |
| | aaggcctgactctgctgtgttcctgaagcttttttattctttataatttccctagctgtacaatggagataatcatagttcatactt |
| | tatagagctaatataacatttaaatgagaaaatgaacctaaagggtgagcacagtgcctggaatacattaatagtaggcc |
| | atcaataagttagggagaaaaaccacagccagaggagtgtatttaatgcacaagtataggagagagaaaaacaatgag |
| | attttgtgactgacttgataggagatgtgaattaaagttatagggttaagctcacttccaggtttccagcttaagccacaaat |
| | cgacattaacccaaatagagaaatggaggtttgtaaggcatgataataaattctgtttggaacctgttcagtttgagatagc |
| | tgtgagacatctggatatagaattaaaatatggatctgagactcagaaagctgtctgggctcgggtaaagttgattaagaa |
| | gttactggatgagtctgggcatggtggctcatgactgtaatcccagcactttggaggctgaggcaggaggattgcttga |
| | gcccaggaattcaaggctgcagtgagctatgatcttgccagtgcactccagcctaagctgcaaaacaagaccttgtcaa |
| | aaaaaaaaaaaaagaaagaaaagagcagtcactggttgaagctgaagctaagaatattcacggcattgcctaaggaaa |
| | aaaagtgaaaggatgatgagtgaagaggcaagactcacacaactaccaatttttaagtaaagggaacagagaggatg |
| | atagacaatgaagttagaacaattacctagatgccaaagaggaatttccaagatgctaattaaaagacatacttgggattt |
| | aacaattagtccaactctctgcctgtaaaataaatgaagaacagtcccaaagaagctaagtaatctcctctaaaatgtcta |
| | ctgacaaacacaaattatgaccttttctattatagttgacatatttgtcttgaatcttactattatatgatttgctctaaaagatcta |
| | agctgtatataaataactacagaaaaatagatttgactagttcatttcagttttctagcctcacctatcagtgtactatgatgct |
| | aacataatcttatgaaagtacttgaagccagccaatacaatgagtacaagactaatatttgttaatgaatcagaatctagaa |
| | aaatagcattgtaaacatttcggatatgctgtggtgttactaaggaaaaatttatttttttccattagtcatggcctatgggttct |
| | catcttgcctaattttccagaacagcagacagtttggcaaattgtattggtccataacctgtagtattagtagtgtctctctttc |
| | cctctctctgtcgggatttttatgattgtgttgcagtcaaaatcgattagttctggtctgtttccatggaaacaagattgtgtca |
| | gaaggctgtaaatttttcatctttatggatggaaagttgctgtatgtcatacaacttgagatagcattaaaaaaatctgtaatg |
| | tattttcacgtctttgagaaggagagtgtgagttgctgtgaaggactgggtccattactgaggagggatgctgtattttcttg |
| | gtatctacaagagactattatagtaagggtataaggtaaaagacaagattgaccgatgaattacctgttgaagaaaattag |
| | ttatttttcccagtgaaattttcactgagagctaatgattgtctgttatttaccatatattctcaggcagcatatgcacccaaca |
| | gcataagatttatcaaaggcatacatagagcactcagcacattataatgataaccactggacacattccaaggcagaatt |
| | aaatactattttctataatttcaatagttttgaaataacaccttaatcagggggctgtgaagattgaataacaataaacaaaat |
| | tctattgaatgattactatatctcagacactgtcatgccatatatgcatgttagcccatttaatcactgttacaatttcatgaggt |
| | tgataccattaatatttccactttagagatcaagaagcttaggtgcaggtagactagataagacaaggagccaagatttaa |
| | acctgtctagttcagaaaaaaaaaaaaaaaagaaaaaaaaaaaaaagcctaggtacctaagcctgcaaaatgagaag |
| | attcttgatgtttgctgaaacaaaattaggataaatcagctaccttactgggaggttactatgtataaagccctgtgtcagga |
| | atgagtggtataatctaagaaatgtagcaatatagtccttatctttatttccacataggagataaagcaaacaggaatgaatt |
| | aataacaaacataaaacccacgtctactttcacatgtatgctacacactttggagttatttgatgataaattaaaaacctagg |
| | ctagcttcttctatgtcaggtacctcttgctctcagtgtgttgtcaaggaaatcagcattactattagtctgaataaatctattg |
| | agaaagtaactctacagcaagggtatcactacgttatagccaaacacagatgaaatctagaccttctttatgttattgttcc |
| | gtttctattagctaaacacatgattgccaacatgtgttaactatttaacgagatgatctatttctgtttttctgtacctggactca |
| | atgtcatggctctgaaaaaaaaaaaaagcttagtaaaaaaagcatgggttcatctctatcttcaagataattatcctggaac |
| | taattaatttttaggggttgatattttctgtgacttcgtccttatcagatagttattagcttgaagagacaactgctaactagtaa |
| | caaaaattaagtcaacattttaaaggacaaatcagcatcactggatttattatctctttaatgtttctattaaatgcttgcaaagt |
| | aaactgtgtgtttgaccccccagtattcattccattccagatgtttccttcaagtcccttgcccatctttgtttaaggaccaggt |
| | atatgatctaattctggccaaggaaaggtgaggggacttcagtcaagtcattgtctgtaaaaagtttcttcactcccaagaa |
| | agagaacaaagagaagatagcattctctgtcctctggacccttttctattgggataaaatacatggcactacttcagccaa |
| | attaaaaccagaagagatgccaatctgagagcaaagctgacacactgagggtggcaggtcacagagacatatttaccc |
| | ctgggcctttgatggatcactcagcttgtggacccgaacaacctctgcactgcttgttctgttcaataaacatctttcttgact |
| | aagacagttttagacagtttttctagttagttgcagatgatacaccaaataaataaatagtctttggtcagttcaaaaacttga |
| | ccaattcagaagccagctcacaaccaagtactcaccacatatttggatgatctttaccccatacagtcaggtgagccata |
| | gatccaattaccaaataaatataataaaatttaaagtaaatacagtccagggtaatttactaaatagacaggatgaatgtct |
| | ctcttaaaagaaaaatatataaaaaactcagttgctgggcgcgatggctcacacctgtaatcccagcactttgggaggcc |
| | gaggcaggcagatcacgaggtcaggagatcgagaccatcctggctaacacgatgaaacctcgtctctactaaaaatac |
| | aaaaaactagccgggcttgtggcaggcgcctgtagtctcagctactcaggaggctgaggcaggagaatggcgtgaac |
| | ccgggaggcagagcttgcggtgagcgaagttcatgccactgcactccagcctgggcgacagagagagactctgtctc |
| | aaaacaacaacaacaacaacaaaaaaacaaaaacaaaaaaaaaactgagttaatctctctttcttacatcaacagccttt |
| | ggcatcattccattccctaaggtcatctgtgctatgtctccctgtatgtatttgcctctttggaagaacaaactggtagagga |
| | aggaagaaaacacccctctgccttcttttggtcctgatacagctgtccttgttaaatagtagagattttagtttattttccacaa |
| | aaacaaacttatacccagaagttgtttagttaccttagatagtcacagcacctttttccggaaaagctccaggtcccttctgt |
| | gaattatctcatttattcttacaatatccctagaagagattctttgctcctttcccagtaagccactctactatgaatccttgaca |
| | ggtatctcatccctgtcaaaccttgatgtggcttggcttcagttttcttgctccataatctctgtagaaagagaatacaggtg |
| | aaattagcctataatctttgacatttggcataggggtgctctggctctgtgtctgggttttattccttttttttttatggccctgga |
| | tcgagaaagcaaggatgatgcaaagcaagatatttgacccttgttaaaagcatgccctagcatgatttaggaagaatgg |
| | gaccaggaaccactgccaggatcctagggttgggacaggggtgctcccccttctttggcagccttgatccatttagtcttt |
| | ctgaaggcatgctggcactcctgagcatccttctgttcttccctcttgcttccttctctcctcttccctccttacatcttccccttt |
| | gcttcttttcctcaaacatctcaaagaccaaagaggtagactagtggaacatgtggttcacattggtgttgtaggtcattttc |
| | tagttccttctcctctccacctaaaacccagtttgattggcttactaaaataagataaataaacctatttagtagcttctaaga |
| | gtatattttagacaagctgaggtaaaaagccagcatgctaaatattaacctaaatgtaataaccagatgtcatacttctttcc |
| | gctataaccaacagcaatggcttagcagagttaaaccaataataataatttcaaagggcaatatatataattctaaaatcag |
| | taactgacatttaaaacaatatttactgcaattcatttatacaatgctcataagccaaactagtcatgaattctcaccagtaaa |
| | tttcccttggccacttaccataacaaaaaaacattaaatctcacttttacaattttatacatggtctatacaatctttgtctccag |
| | atgaactctctcctaagccatcattttttttctctattctgttaccggagtgatggtttcaaaacacatatcctacccttgttcaa |
| | atctgtcagtggctctcagtgactcagaatcactttagcaaagtatggaagtgccttcatgatctggcctctgcctctcttct |
| | cttacctctcaccaagtggtaacaataacacaaatgctaataataatacccataagacacaccttgcatcagaaattgagc |
| | aatagattgttacataaatttataattcttcaataaatgttagttccctctacttcacagccacaagcttaataaatgttatttccc |
| | ttgtcttcatagtacacaagtaactaagttttaaatactttgaccttcatcctgagctataatgaacatgatgttaagtatgttaa |
| | ataaccaaaacatcaatttcaatacatttttaaaatatttgagagctttttgctgggatgtggagggggatgaggtagggag |
| | ggctggatatcgaagagggaataaggaggagcagaagggggagtttgaaaaagcatcccagaagtttctgatttgctt |
| | gagttatctttctttttcctacccttatgaaaattactgatttacagattggagaaaatatattttatggccttggcttcaaagag |
| | gagagagtatttttttttctttttcaatcccaagtatccatgggaaatcgcagtgattcctgcatgtggattaatacttcacttta |
| | ggccacatacttcaaactgcgttatatcttaataagagttgcatctcacttatttttaaaagcagccctggcttacaaattaac |
| | tattatataaaattggactctaagcaattctgaacttcagtgagcataagaatcacctgtgcagcagggcgcggtggctca |
| | cgcctgtaatcccagcactttgggaggccgaggcgggtggatcacaaggtcaggagtttgagaccagcctgaccaac |
| | atggtgaaaccccgtctctactaaaaatacaaaaattagccaggtgtggtggtgtgcacctgtaatcccaggtacaggta |
| | ctcaggaggctgacgcacaggagaagtgcttgaacctgggaggcagaggttgcagtgagctgagatcacgccactg |
| | cactccagcctgtgtgacagagcaagattccatccgccccacttcaaaaaaaaaaaaaaaaaaaaaaagaatcacttgt |
| | gctacttgtaaaagatgcagatttctggattcaatttctggagattctgcaaaaagtaggtctgaggtagggtcgagggat |
| | ctgcatttttaatacactccctaagtgagttttgtgcaagcgatgcatggatgactatttgagaaaggatgaactaaaggaa |
| | tgagtaaagaatatgatctgagtgcttttggcaatcaagcctggaattttggtgtaatgtccccagtaactaagaaaaaatt |
| | gaagcatattggatcacatgaattttgttctattttaggaaaaaacaaaacaaacacatttcctaaaagttgtatataaaaag |
| | caaacatttttcaagaactaaactgaagggtggaaattttgtttaggatatagtataaaggacatgaggcaacatgctcaa |
| | cagacatcttaaattttattttgcacagatgtgtacaagatgcaaaaaagtctttctctgatggttgcaattagttaaactaagt |
| | gaatttcatttagaaggtgatgagataagatggtccagaaactttgctttcagatgacctaatacatttattggatctggaaa |
| | tcccccaaaaacggttagcctgcatttgaaattatcttaataaaatttcagcattttagtaatttaggagaaattctttggttttc |
| | aaataaggaagtgatagctcaaacagcatgatagaaattttaccctaaaaatgctagatgtcaccagctgaggaatctta |
| | gacaaattaaatctttaattttatttttcttaacccgtttctacttagtattgttattaatgcttatgctaatatcaacaattgaatatt |
| | ctagaggcaagtttgtatcctcttttgtggttattttagtttatcctgaaaatttatttatcctttaagatcttctagttcccagtgtt |
| | gtaaaatttttgtcttactatggctccattattttttagcatcattttgcatctttactctttatctctggccatggaagcaactttatt |
| | ttctttcaattgtttttatcttcctttatactgatgacatgatatacctgcttaagaatttttcaggttacaaccatttgtgtcatgca |
| | gtaggctgtaaaattctggctgttcagcattttgcacaagaacagaagcagatccaaaggggtcatggaggaactcac |
| | aagctttgccatgaggaagggacagctttccatctaggtgggaatgtggttggcttttcaacctctgtgatgaggtaacag |
| | tctcaattaccatctgctcacttggttttaagacttcaggtccctatgaaagaggtaacttcaaatgggcaaatcaccaaga |
| | tattagagggtgaaatattgaaatgttgtctaggaagccaagttgtccattcagttcttggggagctccagagattctccttg |
| | tagtaacttcgatcaccttcgtggtggttcatgttaacttaaagtctgagacttctttctttctttgtgttgtataaaattcatttctt |
| | cctttaaaaatgttgagctcaggaggctgagagctaagtgtacattttgaaagagtaaattcattagcctacagtccttaac |
| | agatgtatcacatacattttctccattttccttgtcttgagattcctattgcacaaagatatatatatataactttatatatataactt |
| | tatatatataacattttatatttatatataactttatatataactatatttatataaaactttatatatatataactttatatatatat |
| | atataaaactttgcatcactttttagaagaggtgagatataataaataaaaatcaagttcctctatgtgatttaaaaggctttttcatt |
| | ctttagcatgattctatacttcttataaaccatttttttctcaaggactatacttaattttataataaaaataaatattaagaaatga |
| | acttggaagcactgtatcctaacattgaaaataatctttcaggccatctagtttcatcaacctagtttaaagcagtggttctca |
| | aaatttagcatcagaatcaccgggctaacttcttgttaaaacacagctttctgggctccaccccaggtatatatggctggg |
| | acccaattttgcatttctaacaaatgcacagataatgctgatgttgccagcccaggaaccacactttgacaatacctagctt |
| | attgaatccaggattttacgttgttttttttttttttttttcaggttagaaacatgtatactgggcatatcactttcatttttgcagt |
| | ttcttaaagatgacaatcatagcttcttcagtctcatttatttattcatttgatgccctatggtgtaatttcgctgcacctggaacctt |
| | gaaatgatttacagcagtattacacagaagcctgcaattgctttcagtctccctgctgccagcctgactcccatatagcctg |
| | tttttaccttgcctgtcagagtgatttgagaacatagaaatcgaatcatatcattcctctgctcagaagcccccagaagcta |
| | cccagaaaaaaagtcccaaatccttacaattgcctaaaaggccttataatctgtctgaccttttggttatctagtggaaggc |
| | acctccttctattctcctcctccctcagttctctgtagcctaactggcttccagctgtcctctgtacctggaagcacacacag |
| | actattcccttattccagtatgctcttcctctaggaagctgaatgactaactcttctatctccttcaagtcttggccaaaagttt |
| | actgtcttaaagagacctgctcacacttctcctctaatctcttaccctgctctgatgtttccaagtcacttatcattttgttaggt |
| | actatataacttacttataatgtttattattcattttatgtctctcctggatagcacagaaacagcacaggggctggaatatttg |
| | cctttttggggtctattttcaccaatgaatctcatgtgtctagaacagtacacgcacatagtagtgctcaatgaatactttttta |
| | aatcaatgaattatttaattttggtttctgaaaaatttagtagtttttttttttagatttattatttggaaattcagcccaatctttaga |
| | aactcacatattctatttatggcaacatattttataatagcaatgtctagaaaaaatgaaaccatccactaatggggtattgat |
| | taagtaaatcagataactttgttttgtattatatagatattaagatgataaaacataaaaggactcaatataggaaaaacaaa |
| | gtcaaactttataggcactatatttattaattatgtagccttatttaatctaaatcaaaatacatacattacaatcctctttacctc |
| | aaaaataattatataactatgccaagtttactagactctcttgaaaacaaagcaaattgaagactgaatactttacctaaagt |
| | atttcccactctaaaatatcttaggaagtaagaatgataatagagaacagtgattaagcactggtcacttatcagggactg |
| | attacttcatttaatattcataacaaatatataagataaacatgattgttgtttgtattttttagataagaaaactataatgttcaga |
| | ggggacaagtaacttgcccaaggtcacatgactagtaaatgttgccatcaaattctataattttttctttgggaaaattaatc |
| | aaagcctctctacaatgatcttaccaatgaacctctccagctacatcttacatactctgtatatttccattccttaaaagacctc |
| | agcctctgacatctggcgttcaaaaatgttcacgctgtctgaaatatcattgaccaccaactgtcacctcttaaaatcctact |
| | aaaatttttacagtaattatatatattctatcttctgttgttattgaagatgtgccttattgcttctacatttattattaaattccttga |
| | ggagaaggatgtattgactacctaccacagtaagcagggcattatcagatatatggtggatacaattgcccaaacaaatc |
| | ttgcaaacattctgttcaacgtggtagccattgaccacatgaggaactgagcatttgaaatgtgggtaggtgccaaatttc |
| | aaaatgatatttaggacatatcatcttaaacaaaatatatcattaataaaatcaatcttattgttttctttttttgatattaaaatcta |
| | gaacatttaaaattacatacatgggactcattatatttctaacgcactgtgcttttttcagatatacctgttccctactccatcac |
| | tctcctccaagggatcaaacccatgaacaaatgcatgaattaagcaagaggcgaatcccttattctgagatgttctacatg |
| | ggggagggggctgtctctggatcaatagtttttctattttatgtagtcaggttgctgcttttgtcttttggtctaagaaacacca |
| | tagtgaaaaagctacttggtccataagtgaacattctctctctgtgtgcgcagtggagcagaaaccacatgcaaatcaga |
| | atataacagcattacaggaagctttctattttgaagtacattggcaacatgacaaatataaagcaaacagcattctctgaac |
| | aatccttgatccctttcctaccccaacatcagccataatagacagccctatgtgttgccctcagccacatgtctcctactgct |
| | tgctggtaccatgtgctttaggcagtttctttccaggcaaacacagactgtttactgactgtaatcaaaatgacagcccact |
| | gttttcacaactgagtgttgagagtcgattgcctttttcacttggcaacctgaggaaaattgagaactgggaacttgaggta |
| | tgacttacattacttcaagatggcggtgttgactactccacatgttatctgcgctcctttgagatttaaaatacagaggattg |
| | ggatttgtctagctcagagcaaagaggttttaatcactgctttcagatgggtgcaaatggtaaaccacactggagcacag |
| | gatgatttaggtgtgtctgaagttcagctgccatttgattcttcatcagcaaaaaaccaatcacataaagaggccttcccca |
| | ctaagcaggccccttagatacacagttataatgttacacagtggatcactccaagatggtgcctcagaagataatgcaat |
| | atactagaaaaacatgcccctgccctatctttattagaaatgagatttaatgaaaagataagagagaggaaaacagaagg |
| | agagatatacaagttgcaaatcagacaaactattctagacttgtctctgctgtcacttgtgtgatatattgaacacatgagat |
| | ctcagatgtttcgtgtttatgtgagtaaaaaagaagggagaggtgggaaattgaccagttaatttgtagtacctcttttatca |
| | ctaaaactctagttcagtgtccagactttgggactctgtactaggcttactccaaatgtttgtcccttctgagagaaacttctg |
| | agggtatagcaatacccttataccctgaacgtgggtcactaaaccttatataaatcatgttcctgaggtaggtgtgtcacat |
| | ctattagaggatatattcatttttagaagactttagtgaactagatgcttgcataggtcctctgcagcccaaatttaaactgttt |
| | cttgataaataatataaatgccttgagtagaatataacttgaagatcagttgctgaaaagaactatcccttgaatcctttctaa |
| | ctctagatattcaattttgtacacatgacatcttcatgaaatggtaatataaaaaatctcctactgagaaattccatacaaattg |
| | cagttaaaagaaaaacctagaaaactttaagtctggttttcagtgaaatatgaccagtcacttgaccatatctcccccaaaa |
| | agggccctttgaaataaagaaaagatgaaaaaaaaaagtctttcctgggatctgaattcaggacccagagaatctgtgtt |
| | tttccttcaggctccgagcacatcattggcaccaaaatagagcaaaggactgtgtgcactgccctgatttaggtgtggat |
| | gatgccacttactgcagcggggaatgcagagtaaaaggtctctgcacataaggacaggaaggggatgctgttcagtgc |
| | tcaattcttttgaggctaaaggagatccagaattccacaggtaagagaaaattatatatgataaaaaattttgttttaaacag |
| | ggattgtagcaaccaaactgctgaagactggcgtggtctgtctgtcttcaccctccagaggatagaggacctggtaagt |
| | aagaaactgactagtgagggcagtaatcctgaggagaaacctaagaagaggtgagagggaagatgccaggaggga |
| | gagatgccctgccgtgtgccaggtgtgccacctgagctgtctcctcaaggacatggaggattcaggatcctgataaaat |
| | tgccagtgcaaaatcagacattgagttcactcattcttcaagtgtcactcctctttaaaaattaacagactttttttagaacaat |
| | tttaggtttacagaaaagtctatcagaaagtattgagttcccacatcgagtgccctattgaatgtcttaaatatctgtcttcaa |
| | aatttttattgagaaataaattgagagaataaatagaaactattttgttaagtgtgaaacttcccaaatttagctttcttgaggg |
| | atgtgtattgttgggggagaggaggaagtgtattattttctgattaaattgatctcacagtgttttctgaactggcaccactat |
| | catagatttaatgggatatttaaaagaaaggtgtcttacaatgacaggataattttaagatactgagtagaaaaccattcctt |
| | accaatgcataagagggagctgactattgttttcatggtgtatcttaactctagtgtttatttgcatgaaaatctgcatcttgat |
| | ttggatgtgctctgccacttttcatttaccttcatgctttaccctttccttttcaaaatgttcaaagtgcttttgaataaggagttg |
| | gaatgtttcctcaaaagaatctagctagttcttacaattgtcatcatgttggtagaggggaatggttatatcaaccataaatta |
| | ttccccctctgcactgattgtgttatttaaggtgacccttcttcataaaagcattcctcccctggttcaggctttcattcttatca |
| | atatctcattggtttcttatttatattttcattgcagacttcccaaatcctttgtgtataaaggttggatttttttttttaaataaatg |
| | atccaatcccaatgggaggacacaagcactgtgtcacacacattcatcataaccctggcctcgaacatgataactttgcaat |
| | tcctggccagtatttcttctgtttattacagaagcaccagcgacctgcctcaaaatgtggatattctcatcttgagatttaac |
| | agatattttctgttatgataatcattttactgaccacgttggcagaatgttagagatgggtgaagaaagcatagttaaatact |
| | aatactaaatgatgatcctctagggaaaagactttaggatataatcttctctgaaggacctgtagaggattgccctctatgt |
| | ctcctacccagctgtgggaaaccaatccggaacagcaactggggtgttaacccaggaaatgtctccttaaaagctgact |
| | catcttaggaatgagattctttttaagaaatgcaattaattattaatttcgctccactattagattaattcacttctttctcagatat |
| | gccattttctgaaaatatcttttaaaatgcattttggctttaaaaaatatgttcgtttacagaatctgcctttcctccagatctgcc |
| | taaattgcacttttaaaaaattaaaaatcaaagtgttttgcttccaactggtgatttggagattcaaagaatatctttcagtaga |
| | cctactcttattcgattattcaagatttagttctccagatagcaatttgtaagtaagaaataaatcagtgagtgaatgtatagtt |
| | gtacaaacttaaagttatggatgctcagctccaaatgtgtacattgtttgtatggttagcttgcaaaatataccataaccagt |
| | agctgaatcaactttcaaatatcattgggataataaattaacaatttcaaatgtaaaattatttaaacacataaataaatgcca |
| | gtgtgttcaaccaagggagaaatggacacaagtgtattttttgttgcttccttaagtatttgcataatatctttacttaacacat |
| | gcccgctggttagcactttgaaattattttgaaaagcgtttgtcaactatctttcacttccctagccaagttttcattggcttctg |
| | ttatttttctttgtctttctctagtcaattggttttcaaacctggctacacatttgaactacaattgcagagcttttaagcaaatatt |
| | gatacccagattccgcctcagagaatctgattgaattggttgaaggtggagccaagcattgctcttttttaagttttccaaat |
| | gattctaatgtgcatccacagctgtgggccactctgtgaataagctgttaatgttctacccctttttgttttctttcagtatgtttg |
| | catatctctttctctttagctgtgttttttttttttctcctgacatttttattggtgaggtcctataaatctatttttttcttttctc |
| | attttgcttttttcttttttacctcctatttactggccaagaagttctcattccaggtatatatatttgatttatattcttatcccata |
| | ctattcatgctttatgtcagtgtaagagaacacaatatgatggtgaattatatttttgcctgttcttccaaagctaatttaatagtttac |
| | attaatttttggtataaatataaaaagatcaatagtagaaactaaggccattgtcaaaaacggatattaaggaaggatttattttaaat |
| | gtagttattattggctatgacaaaagacaataaaacagtttagaattttgagaccatacaggtctcaaggacatgtatatatc |
| | atatcaacagtatttttccctttcagaggtaaaggacggcaatgtgtttgccctaaattttaaactttaaaaacaaatttcaata |
| | gtatttaaagaacagagtggaagtgggaatattacatattgcaacaaataacatagcagaaacaagatatcactagcaac |
| | agaaaatagcaatagaaaaaaaagaaaaaaaaagaatcaatgctgctaagtattgtgatgatgttaatattgggaagaaa |
| | actagaaatcaagaaggtaatgcatagattcaaggatttgaaatgaaacagactgagcctagactaggagaaaaggaa |
| | tctgagtgccaatgtagctgtgcttctaaatatggtcaagaggtttcctctccatgcggtttattttccccaactggcgaaaa |
| | tactggttagattggcaaaaggagaaaaagaaaccttgcaattccttggccagatcctgcctacacatgtgttttattgaat |
| | ctatctggtattataaataatttgaaatagatgattccatttaacaacagagggaaatatttactaacaatttggatttttcagct |
| | tcttcagggaagtcatttgatgtaacagtctcctagcagtgagaaggcgttttccattttaaaaagggcatgcattcactttc |
| | tagttcctcaggcccaacatgaatggctttactgctttctcttacctactttgcccatgcaggaatttatgttcgtttcttctaga |
| | atttattatttaaggtcatgaccaaccattatataaattccatgaaaagaaaatgctcagagtacttgggaacatgtttagtta |
| | ggcataataaaaacaacttatatggagaaaaataaatgtcacaagcagaaacaccaaggagacagagaaactaacaa |
| | acaggtgactagtcctcaaatagaggcttaagcccacaaaacgtgtcctattaaaccttatgcaagaatatgttggactct |
| | agaccagaagcgcaatgtcacttgttagaaatatgaattggaatttagtgaattattccagttcaagctctttcttgggaaag |
| | gaaaaggacaaaatgttctgtacaaccaaagtaagaataaagtatattctgaaaatcaggagattaaaattcccaaaagg |
| | gagcttcttggcagaaaatggtcaaagttatttaaagagtgactttgaattcacccattgaataaaacaatattaaaccacta |
| | tttaataattaagggaccatcatttctctaactttgctccagcacagagtttaatcacatgaagggctccctatatgcagtctc |
| | caaaaaaatgaaaacctaattcaattcttacagaaataactccagatttcataaggtttctctgaactaaaacccctgaaaat |
| | tcatttatacaaccacacccagcatatgtttacatgtgctgtattttaatattgtaaacttgcttgcaagttacttttcagttgttct |
| | ttattcatagatcttgggtaaaaccttctttaaagttctttccagaatgtcctttatcatttcaaatccttacaggaaagaagtgg |
| | gatattaaaaaaagtaataaaaaaaaaaaatacttggcacattgaaacttgtggtatccagttaagtaggtattcatataaat |
| | tttaaagaattattctactttaacaaattatatttctgatgtagattcagattttaggtatccaaagagctggtgcctatcagga |
| | agttaataaaacaaagtatgtctgtgcttagtaaaagagcaagattttatttattgctgtattattaagaggaaacactattctg |
| | ctccgaaataggtgtctgcacttgttacatcccattaaaatataaacaaagaatgtgttaatttcatcccagagggaaaaat |
| | attttcaggtaaagctgaactaaaacagtagcttctcaatcaaagataaatgtgtgaaccgacaatataaagcaagaatat |
| | gattttctttcttccaaattttctaactacctcaagttacaattgttcagtctacctcaagaagtaattcagatgtcaggttctata |
| | tggatttgccatgaaagttgtttataaggtaaaacgtaagcatgacattttgaaaatgaaagcagttagaattaaaattaatc |
| | gaaaatttaaaacgaacctaaaagtacagaaatagagttaagtaattatatttcactggtaggtgggtaattaatcctcaca |
| | gttttggaaaactggtaaaacaaccattaatattaaggtgcgaatgaataatatatttttaggtgacaaatgaaacatgaatt |
| | cccaaccttcagtgagttcatgtttctatacataaatgaaaatcagaaaaaagtaatcttcacagctacctatgtaaagaaat |
| | atcactatataatcaagtgttaatttcttggtgaaaaaaaatctttaaatgcttattcttatggatgcttttagttatcatcaaccc |
| | taaagataagaactcgtaagagctgggagtgacctagaactgagagacagatgagcaacaaattccaagcaccgcatt |
| | tgtctagttgagaaacactaccctcatatttttaagcctcccaagctcttagccctctcagatctgcctaatcaagagtccatt |
| | aagtcatcttggctgccatgaagatgaagctgacagcgcagttacttgagtcagtttaaagaggcctagggtggttgaag |
| | ggaaggattaggaggcctggggtgctgaaaggtcatccaagttctagctcccaaagaccccagtagcacacagctgtc |
| | aggagcacgaagctacacttagaaacaacctttggaggaaagcagggattttgcagtccatcaaccattcatcaagcac |
| | aatttgcctttccacatcttattgtaccgttaatagaattgaagcattagagagtttgtgagttgatcacatagcaaagactttc |
| | ctgagatttaggttcagttatctggtggtcttagtagaaattacttggcgtttaaatgtggttttggtgaaacctagattcccttt |
| | gtgaatctttacagaaacttcgtctaacaggtatatcctcttaaggcaacgtgaaataaggccctttgcctctattttcataag |
| | aagaagaaagttttgaagtccgtgataacatattgggcataaataactgtcttctggaaggcattatgagttctataaactct |
| | ccaggaattgcctggaatgttaacactaagtgatactattataggtacacaattgttgttgtttcagcaagggcatccttcca |
| | ggatgagaattattcacttttcaggtacataattatttggcagttttattctatccttcctgttgtggtggaagatattatatcacc |
| | agcactaacgataacaataataacaatgatgaaaatttatgcctttatgtttgtcctgttccttttgatgtgagttaggatggc |
| | agggagagagaggaaggaggttcgtaatcatgttccaggatctataccaagcactgaacagacattattttagttacaca |
| | atcctgtaacacaggcattattattcctattttaaagatgatgtaataaaggctcagagagtttaaataacttgtcaggtaata |
| | gtggccataaaatgtaacctgccaagctcttgctcttaaccacttctccatgttgtttcctgcttgtttccactacagaaggaa |
| | ctggccaagaaacgcccctatttctctattttaccactttcatgtttagcacatcaatcataatttacaactcagtgagagtca |
| | aagttgtatcttaattaagctgagaaaccaactgaagaaaaatccaaataaatgaggatcagaaaattttaaaaacaaagt |
| | attcttatacagctttgattctaatttccttcctttgtaggcaaggaaaatgctcttcagtgatacttggggtcttttctgagaca |
| | acaaaactcactagtagcagaacatgaaccagatcccaagactttcagctcagggttcaacctttttatcttcagtactcaa |
| | ataaatcacagcatttttctcaactttacaaatgaaatcaccactacattcttttaaagagtaagttattcaaatgtgtttgatat |
| | atcatttaatcaaagcaggtttaatttccgaaatttttgaaagagcgcattttgtgaagtgaagaactctgagtgtatttgcttt |
| | ttcacatggcgaccacatgtatagatgaataactaagagactgttgcaactataccaagaaaaataaaagctaacatttgg |
| | ggccaaaattaagcattcattcataaatgtaaaccagttcaatcaagaccagctctgatgcttactcagaacaaagttactc |
| | acatttagtcacctgtagagaagcatggtgtgtacaatacactcaagttaattaacagccatttctgggaaccctgtccaaa |
| | gaaatagttttctttatctcctatggtaggaattcttctgaaatagacaagacccagggctctcctctcaggcaaccactattt |
| | tgaaatgtttttggggggactattattgctactcaatgtaaaatattaagtgagatgtcaatatttttcaacatatcattaggaat |
| | tacagtttggaggccttaagttctatttttgaagcccttttagccagtactgactggaagccctgttgaaatcagaagtcttc |
| | atttcatatagggcacactctatttggagtgaatgtgatgaaatgtttccatgcaaattcctacaagcacttatgcctttatctt |
| | tgtcctcataagccacattatactacctgttgtgtattttaaaaccctgtatgatttttaaaacttatctttcaattaatatgtatag |
| | attttctgtcaataatttcttattctaattatgtggtggctaactgaatgaaacatttatggttatgatatagaatattctaaataca |
| | actacagacaatatggatcatagataaagtaaattattgttttatggctccagtggttcttcctggtaattaataaaaataaac |
| | agttattaaatatatacagaaaaacagcaatctatcaagcttatagaacaattaagaatatataataatctttaaatcaataaa |
| | ttaaaaggtaaacatttaatataaaatgaatatattaaaggataaagaagtagcatattatggaacatatagagattattttatt |
| | ttttgaaggttcaacctcttttaaatggttagtattggtgttcttttctctatgaaaacagattttagagaaaaatgatagtgtatt |
| | tcacaaagccaaacacctctatattgttatcactccctatatgcatggcctctctaaagctgttaaataaaaaactaaagca |
| | caagaatgatagttccaatttcactatacatttatgtttaaaaaatactggacacactgggcatttaaatagatgacatacag |
| | ccaggaaactcattagactagctttaagtataataaaaaatattttttctgaatacaattaatcataagcatctacaaaaacac |
| | aagaccattgatgcctagtatgtaccataaatcaccatttccagacaggctctattttctttgaaagataaagaaatagtaag |
| | cagtttacacatcaaacgatgcctttggttcctgtaatttacatttaaacaacactgggttaaaaatagcagcatgaagatg |
| | attttaaagtaaggaaaatgacctggtagctaggttctatggtgcagataatgaaatgttttgtcagcttacctgataaaaca |
| | gagccttatgaattacagcataacaaagcccagcatccaagatggttagggtataacgtgtcttcctctgcagctgttcag |
| | cttttttttttttttttttttttgaccacagaggtttacaaattagttacaattttttttctttctttctctgtggataagaaaacaaa |
| | gagaccttccagaatcctctctgcttatgcctccgcttcctgttctgagattcactctatggtacccaacatatatcagaaaaca |
| | gctttgaggaaaaaaaaaaagaaagaaaaaaaggccgatcgcactcaaagtggtaccgccatctactggcggacaca |
| | cacaaaattgagccaaagaaaaaacattcccattatcctctgtcacagggaggaaagtaaacctgcatgaagcatgggt |
| | agtcatgaaagtgccccaagatgacagtagagaggtgatgtgagctaaaatttcccaacacactaaaaaaatgtaactta |
| | tctactacatataaatatgtatgagaccttttacatattcagttatttgcatctcactaatttttttaaaaaattttatcactttgttgt |
| | tttgcatgattgtttcacacataaactactaaaattaaaagatatataaattactgtttgaaaaataggttttaagtttacattac |
| | cctttacgtgtagagttttgcgaaagctcccttgtggcctgctctagtcttttgaaaaccttttctcttcctgggatgaattatttt |
| | caaggtgaggaatttgactccttagagaacagtgtaaacacatactcactgatagtatttttgtgttgctttctggtaaaacat |
| | ttgcaagaaatatctaaatgtagaaaaagattctatacatcagtgaacctagctaatattatttattaggatattgaagcaca |
| | caatacataaagaaaataaaaataatccatcagttattaagatatttctcaatccagatgtatgttattgtttcatctgacctttc |
| | atatctactttagagcaaacaactatgattttattacagtttatcatcaattaaataaatacattcatattgacaaaccaactgta |
| | tggatggacaaaaatgatgtcaagtattatattagacttgaaaagacacagagagaaacacacagtgcctgccataaag |
| | gagctcatacaaagaagaaaaacaaatatagctgataccatatttcccagccatgtgttcaagaactgttaattacatactt |
| | cgttttcaattgtggcccccattttgtctaatggcagaaaaataaatctagtcagttccaggtaaactaaattacttgaggctt |
| | ttcccaatgattttgggtttgcattcaaccaatacatgattttcctcttctatctctccttctcatggagactgatctaagttcag |
| | gattgtgtacatatatagagaaagcagagtatgatcccgttccccctttctccacatgggcatccactgaggccaggagt |
| | cctgcatgatccatgaccatcattatccctcatatctactaacgtacccacaggcccactgggccccgggaatagcatcc |
| | ctccagctttgcatggcagcatcccctctttgtcacttcttcctctaaggaaacttttgttcctgtcctttaggaggagagaag |
| | aaaaagcagcatgttcccagggtcactcttggctgtaattggctgacttaagccttcctccctgccccaggcgcctcaca |
| | cagccatctctgttctgctgttttgtaaaccatgggatgtaatgcactctgcaggcttcaggcaagcgaggcacaatctctg |
| | ccacactagaatagtgtaaatagatggaaacttctcatcttttcatctttttagctaatcctagattggcaaactatgtaagga |
| | gcccttatcagagactcctttccttcttcataacttccttcacagactcaaaagagtttttgcttgggtctcttctttctgtacca |
| | aaggtaagctggagccacttgttttcccttgaaatctttttgtcttatgtctaatgtatcagtttactagggctgctgtaacaaa |
| | ctgccatcctgaggggcttagacacagaaatcatttttttcacaattctggctactcagaggccagaatcaagttgtcagc |
| | atgatcatgcttcctctgaaacctgcaggggaaggatactttcttgcttctcttagcttttagtagccccagatgttccttggtt |
| | tgtggcagcataactccagtctctgtctctgtctttacatggtgcactccctgcatcactgtctttacgtactcctccctctgtg |
| | catgcctctgtgtcaaattttctcctctttataaggacaccagtcactttggtttagggtccgccctcataacctaattttaacc |
| | tgattatctctgtaaataccctatttcaaaataagggcatattctgaattactcagaattagaactttttgagaggaggcacaa |
| | ttcaacccctaacacaagaatttaaatataaagcacaagaatttgacaattcttctgtccatctccaacgacaattccactta |
| | ggcaataatagccttggctatctggccagaattggggagggtaggggaagagcctcaggtaatgatgaagacatttaat |
| | aatattagtaacaataacaaaactaacatttattggtactatgtgccagacattgttctaagcactttacatatgttcatttaaa |
| | cttcacccatatgaggtattattatacacactttacaggtgaagaagcaatgaaatgtgagggaagtaaaacgtagttagt |
| | acctgatatggtttggctgtgtccccacccagatatcatcttgaattgtcagatatcatcttgaattgtagctcccataatccc |
| | tacatgtcatggggggcacctggtgggaggtaattgaataatgggggcgagttttgcccatgctgttctagtgataatga |
| | ataagtctcatgagagctgatggttttataaagaggaacgtgtcccctgcacacgttctcttgcctgccaccatgtaagat |
| | gtgcctttgctcttccttcacattccaccatgattgtgaggcctccccagccatgtggaacagtgagtccattaaacctcttt |
| | ttctttatacattacccagtctcaggtgtttctttatggcagtgtgaaaatggactaatacagcacctcacatctcatcccaac |
| | acaagcagttgtaatcgggtaagatcactcataatacaagcatttgtcagatgctgaaagaacacaaaggaaatcagctt |
| | ccactaatttagatcattgatactcacacgtttagtgtaaatttataacatttcattgtttcattgtgtacactttctgtcatgaaa |
| | gattcctattttctaggcacttttaactttttcttctctactcatgaacaaactagccctgctcactgtctctcaactaaatgttca |
| | ttaagtgccagtccctactgctccacactcattttaccattactagtaatatttaatagaatattaattctatcactcagttttgg |
| | gtatccagatgctccaaagagttttctgatgacgtcttctgaggaactgatttttcttaggttatactgggccacattttaatga |
| | gaactggtttgaacaaaataagaaaagtaaatgataattattgtggcgatattcctcatagaaaaaaagaaaaataaataa |
| | atgccagcaaagagagctttactctttgttgctgaagaaactaatttatcttgtcattaaaagtagaaaattatataaaggaa |
| | gagtgaccagaggtgtggtatcatttgctatgattctaacttcattgtgactttcgtactttttttctgtagacagaacgacag |
| | gtagtttgcaaatcttcagtgagtggattctgagagggttaaggtgaaatagtcattgattaccaacataaggtctctggag |
| | ccttagtggtgtctgaccatatgttgtaaacatatcatgcataaatcacagaaattcacactagaagagagctaatagcctc |
| | cagctgggactcataggttgctctgtgcatgtctactgtttctgattactcaaggtcttcagagcctttgtgggtaggagcta |
| | taatgctggtttctatttgattttataagcttcatagccaaagaatactttttgtaacatccatagatctaccaaaagattccca |
| | gactctggcccaattttcttgtttcctagaccattatttcccagacagtgattggaggaatactactgttctgctggactaatg |
| | aagtgccactccacatccaccagaatggctacaacttaaaaaggcttatcatactgtttgtgagaatctagagctacagaa |
| | gctgtcagagacttcaggtaggaatgtaaactagtataaccacttagaaaaatggtttggcattaccaactgaacgtgagt |
| | ctactctgtgagccagcaagtgccactcttaggcatatacctgacagaaatgtgtgtgtgtacaagaatgcctgtggaag |
| | cattattgaaaacaggtcaaacaagaaagaacagaaatatcttgaacaggagaatggatataagaattgtggtgtattct |
| | gaatctatagtatcatactaacatgaaaatgaaaaactgcagctacccacagaaatatgctggatttcacaaatctgaggc |
| | tgaggacgaagaaggtatgtaagcaaaacattattaaatttaaaaacaggactgggcgtggtggctcaaacctataatct |
| | caacatttttgaaggctgaagtgggaggatcacttgagcctccaggagttcaagaccagcctgggaaaaatggcaaga |
| | ccctgtctctacaaaaaaaatgaaaaaaataaaaattagtttggtatggtggtgtgcacctgtccagctactcaggaggca |
| | gagatgggagaatcacttgagccagggagttccaggccacagtgagctatgatcataccagttcactgcagcctggac |
| | aacaaagccagattcttgaaagataaaaataaaaacaggttaaataaactctaatattggaagtaagagcattggtttctct |
| | tgagataagatagagcttttggcaagattggttgagataagggctattctgggtactagtaagatcctatttcttaatctggg |
| | tagtagttatataggaattcacttcctggtaattcattgagccaaacatttatgcacttttttgcatgtggattgtacttcaaattt |
| | caaagtaacaggcgagttttttgacagttgtcacctctgaggaagaaaaaagtaatgaaaggtgaaataagtggaatttc |
| | caggatttgatctgtatatatctgttctgtttgaactttgtatgcttattagtgtaataagaagtgcattattagtgtaattttcaaa |
| | aaataaatttaaggcattaaaagtacagtaaagatgtaccttcatactatttgtacttatttacctctaagttattctctttttaagt |
| | tgagtctaggatggcattcatcattattttaaattgagtctgtcctggaattcctcttatgccagaaggcctattcaagctgat |
| | actcagcttctatgcttctgtcttcagtcatcttagtaacaaagttaactaccactgtgtttgcatgtatgttaaataaacaaaa |
| | tttacatgtcatctacaatttcacacctctatagtctagtttagatttgtatctcctgattgtaaatactgagactaactcactttt |
| | gcccactgcctaatatattatgccataaattacacaatgaaatacatgacctgacaaaagataataaaacacttttacctttg |
| | ttacatcttcttcctttaaaacatctaaacaagttgagaagggtagataattttcagatttttcaggtgagaaaaattggtaca |
| | gaaatgtgaaaattacatgatggttaaaaattggccaaataaaacaggataattgccagtttaaaatatatgcattagacaa |
| | tatgattttatatcaagggctcatttcagatataataactgtcaaatattatttagaaggtgtgcttgattcagcaatattaaaaa |
| | attttaacctgtattataaaagaacaatagagtaaaacatactttccaagtggcttccagtatagattaccttctgtgctgtag |
| | ttgcacagctatgtaatagcaatttttcaaaggtgttgggtcactatgcctttttagttgccattcctttggttagaaattactttc |
| | ctttattgccattctcacccctatttccctccaacatctggaaaactactatttattcttgaaaatgcaattagggtaatatactt |
| | gatagcttgtcctgatctttccaagtgtaaggtatcaacttaactgtactccctgtcatactgctatacacatcattaatggag |
| | gaggtgaaaatgattcatccattaaaatgactgaatattcttaggtgcttgtaaacgcccattatttaccatggagtttcatca |
| | gaaaagacaggtgtgaagcagataagggcaggtactgggagatacacgtctctctgcttctctcaggaatgatggctg |
| | gataatctgtttacccatatgcacacacactgtctactctagggctttgtgactgagcaacaggcttttttttccctatacattc |
| | tttgttagtatgacactgtcagatcatctaagctaaagggaaaaatgtatttttttttcatgagttcctaacaccttttgctaatta |
| | taagcaagcacgcaggcatttcccttatataatttctcattttatcttctaaaatttggtacatgtgagtagccaactatgtcca |
| | aatctttatatgattaggccaatatgaaaagaattaaaattttaatttaatatgctctcttttattttggaaaatatttacttgcatc |
| | atatttccagcttatattatatttttattggttgtttagcattctttaatactaaaacaataaaataggaaatcatgcttagaccac |
| | gtcttctaaatttcgaagacgtaatatgttgaaaagctttatttcatgtataatacattttcatttagtttatcaattttaaaagata |
| | atttcaactaaaaaaatccttcacagtttataagtgcactttattagtggagaaaatcaagggttttaaaacatcagagtaat |
| | catgatggaaattattatacgttcatatttcagaaaataactactctttttttaattttacaaaggtaaattgacaaatgcaacttt |
| | cccataagctctgaacaaaaagaggggctgttaagataagtatgaacatgggggaatcaggggaactgggaggtgct |
| | aatgaagaaaaagctagaaaagaatcaaagaactacattatttaaaaacttcatctattaggaagtggctaagaagaaaa |
| | atatggagagagatggaatgttactgtcataaacctgggagaagatgatcttatgcaaaagttaatgtaagcggaaatgg |
| | acacaagtatatggatttagaagatattatggtttatgttgtagatatgaattttaatctatatctatgtgtgtgaaaaataatag |
| | atcaacagtaacttcaacttcagtaattactatagggcacctccactcaatgtatcaaagttccagagtcttgtgtaggacc |
| | caggtataaaggtagaggtgaggggctcaatcttttgtcatatatttgcataggttacaaggattcatttatttaacaaatatt |
| | attgagcatctactctttgccagcaactcttcttggtgcttgtaatgtatcagtgaacaaaacaaagatccctgccttcatga |
| | agcacatgctttgggagtggcacaaatgtggacaataaacaataatattaatgaattagtaagtgatattaaatgtcagaa |
| | cgtgattagtgctaagtaaacaagtaaaaattaaaatctggttgaagagatcagaatgtttgatggagtggtggattgaga |
| | tttaagttagttgatcatacggaaggccttattaatatgataatactttaagcctatgaccagtgtagtaagcctagttcagag |
| | tccacaacctaggcttataggagatgctacatttccttatattctacaaatatattttatttagattttggtgccccattcgaacg |
| | aagtggcaagaaatccaaattatttattttagaacttatgaccccctctcccatcaccagtgattaagtttcccaagtcttacc |
| | taatgacattagtcttctctccaaacaatttacttttcagtttttcattgatatgtttacctctctcatacatccgtgtaagatatgc |
| | cgtgattccccatgaagttggaaactggtggctaaatcccttctacttacgcacatcaggaagcagtcctttggtgagata |
| | gtattatctctctagaggtcttccaggaaacaggctctctgctactcagattcctacaggcctatttcttttttcattatttggaa |
| | gatttttctgaactggagcatcccctcctgcctccaaccacttcttcaagacacacagcttctacccccaaaatccatttttta |
| | aaatagactttgagagttcattaaatccttaatttaaatctatggcaactaaaaaactatatccctttcatcattgtcatgggtg |
| | tctgtatcttcaaacccttacaagagcttggggaaattatttatcttctattctgcaagtataccaaagaatagaacataatag |
| | ttgttttcatactaagtatattttcatagtaaaagagtgaaaattcaaattccatcacaatattgaggaggtatcattaaacctg |
| | attttgaaactggatagaacagtgagagagaaggaggaataatggtgaccctcaggcttttgacttgaagaaaatagtat |
| | ctctgtatgctactcatgaagataagtaaacattagctagaccaggtttgtgtctttgaagatgagtcatttagttcagggatt |
| | tctgtgtttaaactacttgtgggacatccaaatagaaatgatcagtgacatttagaccaatttgtagtttataaaagagatcc |
| | caggtgaagagcttgcatcagagaagaaatcatttttccgtttctgttctccctaccattgctccagattctgtttccttcaatt |
| | tctgctcaaaacttatcctttctattattttatattattgtatctccttccaaccttcaatcattatttcaatcttcaattgtggcataa |
| | tgcctctgactatgagccgttattaaaaagaaaacaaaataagggaaggaaggagagtaggaagaaaggaaggagg |
| | caggaagagaggaacggcaggagagaggaaaggaagctcttcactaacgtttgacaatttgcgccagcagcactctg |
| | tttagtcttgtgccttcagggatgacttttaatatatgattcctcaatatttttccagccttaattttaatcttctgattgtcagtagt |
| | gggtcttcaaataataagcatctgtctggctgccatgccaccaccaccaactcaacataatcttaagctgaactggtcatc |
| | atgcttgacaaataataaaaacacaaccttgtcttattcttaataataagtattaaatacattccattgctcaggcatggttttat |
| | tcatgactcccaccagccactttggatcatccatggtccattgctcacttctgcctctgtgcctttacaccattgactttgccc |
| | agcctgaaatctatctttttgcaaggtgcagatcaagtttgacatcctctcttacctccattacctagaactaagcacttctga |
| | tcaataacatgtctgagagccaatggatttttttcaccatgaacttaacataaactacttttattgatttatctacccttttatactt |
| | tgggtgttaccttaagaaggacattggaagtttttctatctagtgtgatcttgttgtatgtcttttttacgttcacagtgcctgaa |
| | agaaatttttcttccactgaaggcttgatgctatgaaatggagtagctcactgttttttctccaatccatcaaattcctttctcta |
| | agtatgaaactcggtaaccactgacattcctattccaaattcaccagctatgtgattttggtcaagttacttgaccacttgaa |
| | accttaatttctgcatctttaaaataaagacaacaccaattcttaccttgtagacttctgagcattaaatgaaaaaaaaatcca |
| | tgtgaagcatttagcagtgccagtcacagtgtccaacaaatatttagtcataatattcatatggtcatatgtttctaatttaact |
| | acagcactctaattatacaacaatggaaaacctagagatgtcttcatttcctcctgctttctcatgtttcttatctgatcaactg |
| | gtgatcatacaaagtctctaatcagcaccatagcactgctaccaccggaatgaaatctttatgacctctcactagagatact |
| | gcaatcattttctgacctaagtgattccccactgatgtcaagcctgcttcctagagcatcattttcaatatcccattctcctaa |
| | acaaaatctttaagaagccccagtctcctgctgtagtcaataaaatcaatattgaactcctgaacctaatattcatgactttc |
| | cacaaaatatccactatccaattctaacaaaccaactagaaaaaggcattttttaggtaagagaggaattttgaatatggac |
| | tagttattaaataaaaacaaggaattatatttattttgctatgagtaataatggtattgtgttatgttaaaaatgagggtatatttt |
| | ggaaatgtatcctgtataacataaggatgcaatgttataaaaatctgggttttgttttaaaataattcagaaagagaaaaaca |
| | aatagatgaatcaagtttggtaaaattttggtaatgagtgcatatgggataagtccattatactgttctttctgcttttgaatatg |
| | cttgtttttcacaatacaataagtaataaaatattataaattatataatccattgcatatgctacctgtttcccgtacttctaagag |
| | ctgtttttccctactctaaatccaaccacattcttttattgctcttttgtcatttattatctgtagatgtgtattataattgttaaata |
| | ttttagaccatctgttagattataaactttatgaaagctttaatatattacttgtccttgtatcccaaaattttctaatgtattttctg |
| | gtacacaatagcttacatagtaaataattgtgtaaacaaatgggcaagaccataaaataaattacaaacatctctgaaagaaa |
| | gggttatttctttgataacaaaactaatgcattttctaatgttgtagcatttctagcctgagacctaattaagcaactgaaaaa |
| | gaagtggtaaaattaagagccactgtcagcaggagtactcataccataaatttttaattgctgaattttgagacaaataatg |
| | acttttaaaaagagtagacagagtttatgaataatgattctcaaaatctacccaaataaaagggtagcaataacacaattgt |
| | taaggggacagaaatcccgtagggaaatgttaattgtgttatcatgttttaagcctggagaacataaagacttagaagtaa |
| | ctttaaacatacaaagtttgtttctattggagaaaaaccataacctattatagacatgctcaactgtatggacagattcaaac |
| | aattcttttctctccccttataaataacactaagtatttgtaacatcttttggagggcaactgtatatttgtttttcttgcaatagat |
| | tagcaatacaagttttgttttttttttttaatctctctctggacaaaaaaaaaaaaaaaatagtgcctttactttccttataaaatgt |
| | ttctctagctggagatataatttgccccaaattggcaacgtgtagtcttacagaaatttctttccctctatcagccagcaaga |
| | caggtggccctagctttggtccccattgctctgtgtcactgcacacctggacctaattgttcagtctcaagtggagcaggt |
| | gagatgagggtgggctgttgtgcaggaaatgaatggacttaacacagtggtttgcttcggcaccaggcagtcttatttga |
| | gtggggggcgggcttaggctatgggaggctcgggagggagccacatagaaggacagataatgcaaacacagactt |
| | tagattccaaaataattcctggcattaaggagggcctggcagcctgcttacatagtagttaaatgctggttaacaccaagg |
| | caggtgctctgcatgggctggacaattgcaagcacttggataaatattagttgttgatttattatcactgttttaagagtgtcg |
| | gtttcccagccttcaggtagattctactaatttattctatctatccccctgtttaataatttattgagttttaagaacaccgcaga |
| | ggaagcagccattgttaaaggaaggtttcaggtcacttgacctgggtagatttgtaaacataaataaatgtgtattgcctgt |
| | ttagctctatttcttaggggcggaagcgtccagataagaatttgtcattcgcgtatgctatttaattattatgacagatcaaag |
| | agacatgctggatgtgttttttgtgaaaaaaaatcacataaactcaatgtaagcaggggaattgagacagggaacctcat |
| | ctttaaacttcaaggaaaaatttgaggctgtaactgtgatgtataaaaaatattaaagctcaggttattcacttagttaacaag |
| | tatttactgaaagcttagggtgttccctgcattctttaggatgatgggacataagtgagaataaaatacagtgcctgatcgc |
| | aacaagctcacagtctatagaaggggactgacaacacaaacaacatggcagttttaacatggctgcttatctgaagcct |
| | gaggtatgcacaggtgctttaggatacctgactgcaaccgctcttcaaagaaaaattatttcttctcttgatgtgaaatagat |
| | aaagcttcaaagacgggatagaaggctttaagacatgtagatgttctgaagtaaatgactttacacagaatgatttatgcg |
| | aagtgcattaattggtactgctagaaagctaatttaaagaaaatcatatggctatattgacacgatgtgaattttagagctctt |
| | tagattgtaagtgacagaacttcagttcaaaaggattcaaaacaaaggggaaataaaagtctcatggagctaaaaattgt |
| | aggagctagaggtactttgggcacgtctgggcccaagtcttcaaatgatggcatctggaatctgttcctctcatcattggc |
| | ccagttttcttctatgcttccttccttgttagagaggttaccggcacatgacatactcctgcaagtcctggctgacattcagc |
| | aagattcaagaacagtgagaatgaatatattttattaccaaatgcttcatcagaaatcccagaaaaacctctaatagcttct |
| | gattgggtctggtgtccaaccttggctaatcccaggagccaggacaatgtgagtctctaaataaccaaacctgggtcac |
| | attaccatcttttgagaagggaatcaagtcagcccacccaaatcatgttgactgagagcagaagagaggtagttacctgt |
| | aggaaattggagagctcttacaaaaagtgagcatggttgctggactttttaattacattttttttaattttaagataattgtaga |
| | ctcatgggtttttactgttaaacatttttcttagatacttaaaaattaaaatctattcactcaaaattactaagctattctctcaaa |
| | gcctgttttatttttttacatgggatacatatgctgaatgtgcaggtttgttacataggtatacatgtgcattggtggtttgttgc |
| | acccatcaactcatcatctaggttttaagccctgcatgcatttgtcctaatgctctccctccactttccccacactccctgaca |
| | ggccccggtgtgtgatgttcccctccctgtgtccatgtgttctcattgttcaacttcaacttatgagtgaggacatgtggtgtt |
| | tggttttctgtttctgtgttagtttgctgaggatgatggcttccagcttcatccatgtcctagcaaaggacacaactcattctttt |
| | tttgtgactgtgtagtattccatggtgtatatgtgtcacattttctttatccagtctatcattgatgggcatttgggttggtcccaa |
| | gtctttactattgtaaaaagttctgcaataaacacatgtgtgcatgtgtctttagagtagaatgatgtataatcctttgggtata |
| | tactcagtaatgggattgctgggtcaaatggtatttctggttctagatccttgaggaatcaccacactgtcttccacaatggt |
| | tgaactgatttacactcctaccaacagtgtaaaagcattcctatttctccatatcctctccagcatctgttgtttccagacttttt |
| | aatgatcaccattctaactggcatgagatggtatctcattgtggttttaatttgaatttctctaatgaccagtgatgatgagctt |
| | tttttcatatgtttgttggccacataaatgacttcttttgagaagtgcctgttcatatccttcacccactttttgatggggttgtgtt |
| | tttttttgtaaatttgtttaagttccttgtagattccagatattagacctttgtcatatggatagattgcaaaaattttctcccattct |
| | gtaggttgcctgtacaatctgatgatcgtttcttttgctgaacagaagttctttagtttaattaaatcccatttgtcaattttggct |
| | tttgttgcaattgattttggcattttagtcacgaagtctttgcccatgcctatgtcctgaatggtattgcctaggttttcttctagg |
| | tttttatggttttaggttttacatttaagtctttaatccatcttgaggttatttttgtataaggtgtaagaaaggggtccagtttctgt |
| | tttctgatactaaaaagctatgtgatattaggcaaatcacttgtgttctcatcttcaaatgtaatttttctggttctattttataggg |
| | ttgttcacaaggttaatacaatgacaatatctttttaaaatggaaattacgactaaaatattgtattgtagcatttctttacaagc |
| | atatatttttaaacgtgtttttactcacatttgtataaagttttatttcttttatattatctctacctcttatgtacacagagaaacact |
| | tgggtttaacttctaatatgataatttcgcctcttacactttcaagaaaattaagatttggattttccatactccttcattggttctt |
| | cattgcttatttgtagattaattagcaagcattatttatattgtcactgacagacagcatgatataagtgatgataactgtcaat |
| | catgcagcttactgggtttttttttttttttgagtgagtggcatgaacatttctcttcactaattagaaatttattctatacaactctt |
| | ccttagggttgaccagtcctgcctttcactaagagacttctcacaatgaaagggctgcctggggcgcactggaaagctta |
| | gtcagcagcggaattaccttcttcctgaaggctgactgaagaagggaatgtggcaatgagcaagaggcttcacaaatg |
| | acagctagtcatttgtgctgggcgtagagtcttcttgcctgcattctaagatggtggggatgacaggaagcaggtctgcat |
| | catcagctcagcacactggcatggaaccaggaatggactgggaggcaagacttttgcttaatgtctgagtttccttaaatc |
| | tttttagaacaaagcaggacaaaaattttaaaataaactagtatatgaccttgaatacattaaacaaaactattctacaattttt |
| | ctagcttagaacactcttttagcagtcattttaaaaaacaaattaagattaaggaagcagtttatatttcttaaattagtttatta |
| | attgttttttcctgagtagtaaagtcttaagcaatgcgtagacaatttggtgagcaggtaacagctataatcataaaccaga |
| | attctcacctcctaaggaccactgccagccctccagagtctgacataagtaaaccacacaattaagcaaattcttgatcac |
| | cccagtgatatagatagaaaagctccgatagaaaggaaagtctattctgattatgatgggttggtgacatggagaaggat |
| | taatgattcagactgtagacaattttggctatatagttaatacatatatacacaaaaatatgtatatttatatattgaaagtgaat |
| | aatagcttatatatatataaatatagagagtaatatttaaatgacatgtagaaacttaagtcctacaattctatgtgtcactgca |
| | aaatagcaagctaactgttgaagaagaaagtattgacagactccattccatagattataaacctgaaggaaataaatgag |
| | gtgccaaggcaagcttctctcccacgtctcttgcataagttgtgggcattgcattttcataacacatctttacaccattttaaa |
| | tatgaatgtcaattgcaggaaagattaaaggcattgtcctaagagcttaattcacagggcgtgtcctaagtattagcaaaa |
| | gcgaagtccagtatgggtaagagcaaataaaatgagattgctgtttaataagccatatttgtaattgaacacaagactttca |
| | agtgctagaactaaaactgctttctgagagctatcatatatcagatggtttccctgagcacttgcaaattaaaagatttttaaa |
| | gatatagtgctattccatttatgtgttttacacagagttagattgatcacattaataatcaataacatttggaaagaaatgatgt |
| | ttgttaagccagcctcaagttatgatttatttatttttatgtagggactttagttggctgggaaaatgactgctcagttctcgttt |
| | gcctgccaaaaagcagacattttagaaacagtgatgtaaaagtcagtaagaattatgtggcttcaagtggcagaatacct |
| | acctacaaccggcttgagcatagagagagattattggctcatgatacttgaaagtccagaggctggtatggcttcagaca |
| | ggtccgcacccagggaatcaaatgaaatcatttggatccatcctctcttccagcttcacattgtacccagatgtgaagaatt |
| | tagaggcttttctttcctcactcctttcctttcctcactaatggctttagtcatagatacactctcctgtcttgtgccacagtggc |
| | ctccaggagctccatgtttacatccttacagttctagttcaattgaaaacagagtctgcctttctttaaacagtttccaagctct |
| | gatattccaatccactggctctgattgggattcatgtccggtcttgaaccatgtttggtgaccaaaggactacagagtctta |
| | accaagtctaactacattacgcatttgtgccagaagctaaaggaagtgtcagcatcacctgggcagtagatattggatgc |
| | ttgagcaagaacattctctagagaaaaattgtggtgcaggcataaggagatgaggggtgtgtgtacagggtttcaagtat |
| | agcaagtgtccagtatagatttttgttaccactggtcaatcacagaagaggatagtaaagtgaagggaggttgtcttatgg |
| | tgacatctaaaagccactaaaatgacattccctggaaatagtttcttgaagaattatgtcttgtagggaatatgtttgtggaa |
| | gctgaacacacaaataagtggacggcccctctccatgacccaaattcagatacgtctctagcaaaagttctatcacagat |
| | actcttactattagtcaatgtcttcctatatcttgctacacctctgattaaaaacaaataaacaaaaaaacttgtctgtatgtag |
| | aactatcaatttttagggtgatttcccatttattttttcatctgaaatgttctctaagtgctttctattttattagctaaactaatttt |
| | actttgtttaaatttcaaaatgatatatagagagtcaaacagaatggcttctaaaatattttagtttgagacttactctgtctgagc |
| | agccctaactacatttttctctttaagatacagttttattaaaccctattcacttcatactaaatttaatactagattattatatcca |
| | aggaaagaaaaatacttaactctactaaccattctgttttattttctttttgtgagatttgactcttttttccagcatattgaggtat |
| | tttgaggtcaagtttatcatgaaataaacttccctattcctcaacccatcacattattatgtagattgggttaaagtgaggctg |
| | caatgatttcctcatcctatgcagtagtttcctctacctgaaggtttcttaagcaaaatatctactcattcctagagccagactt |
| | gagaggcatgtaccttagagttcttgaagccaggatttatgattcagttctaagctgaagatggcgaatattaattgcgaaa |
| | ggatacaggaagaaaaccctccagattcttcacatctgggtacaatgtaacataaaaggaggagcaaaggtattaaattt |
| | gggtaagaaggaaagaatgttaaggacagtctaaagattgtgttttacctgtgtatatacacttttagccaaattgccagaa |
| | ataacaaaaggagtgataaatttcctgtttaatcctgactactgcaaattgactgcacccctactattcctcagaaaccaatt |
| | tctctgggttcactaatgacctaaaactgccaaatacaagaacatattcctgtccttatgttagttgttatttctgttttattttgta |
| | atcttgactcctaattctatgtaattccactccctgatggctttgatgacaacattcatcttaatgacttttctcctatttctgtggt |
| | cctgtttctagtctctcatcatccaagtcagaaactgagagtcagactaaagtcatccttcatacatgttccttctccaacac |
| | attcactttgatttttaacagcttacgttaagagaacttgcatactgtaaagctcactcatctaaagtgtatgatgcagtaatttt |
| | tagtgtagtcacagattcatgtaactatcaccacaattaattttaatacactttatcacccccaacaggaagctccatattcat |
| | tagcagtcactttccatttccccccaactttccatccctgcatccccagccctaggaaaccactaatccactttctgtctttat |
| | caatttgcctattctaaatgttccatatgaatggaattatatatgatgtggtcttttgtgactggcttttttcccttagcataataa |
| | actttgaggttcaaccataatataggacgtatcagtatttcattcattttatcaaatagtatttcattatatagctatatcaaatttt |
| | atctatccattcatcagttgatggttacttaggttgttttcacttttgggggattatcaataatcctactagtagcattcatgtata |
| | agtttttgtatgaatgtatgtttttatttctcttgggtatatacctaggagtagaattgctggatatcttttataagggtgctaatc |
| | ccatttgtgagggcttcgcccttatgacctaaccatctaccaaaggccctgcctcctaataccatcacctttgagattaaga |
| | tttcaaaatgtgaattttgaggagacacaaatattcagtctatggcacatagtcatatgtattagttcattttcacgttgccaaa |
| | aagaaatatctaagactaggtaatctatttttctttaaagaaaaaagaggtttaattggctcacagttctataggctgtacag |
| | gaagcatggcagcaactgcttctggggaggccatggggaacttacaatcacggcagaaggcaaagccagagctagc |
| | atcttcacaacatggccagggtatgaggaagagagatgggggaggtgccacatacttttaaacaaccagaccttgtgat |
| | aactctgtcacgaggacagcaccaaagggatagtgataaaccagtagaaactgaccccctaatccaatcacctcccac |
| | caggacccacctccaacattggggattacaactggacaccagatttgggcaggaacacagatccaaactgtatcagca |
| | tacctactcttctttccagtttgtgacctctgtcatagtttaggacctcattagttttctttttctttttctttttttttttttgagac |
| | agagtctcactatgtcgcctagtctggagtgcaatggtgtgatctcagctcactgcaacctccgcctcccaagttcaagctattc |
| | tcctgcctcagcctgctgagtagctggtattataggcatgcaccaccatgcctggctaatttttgtatttttagtacagacgg |
| | gttttaccatgtttgccaggctggtcttggactcctgacctcagatgatccacccgcctcagcctcccaaagtgctgggat |
| | tacaggtgtgagccactgtgcctagcctaggacctcattagttttcactgggattggggaataaatatttatagacatagtg |
| | tgtgtgtgtgttttagtatatatgtatgtaagtgtatgtgggtgtttgtgtgtagtctcttctgtatatacccatatatatattgctgt |
| | ttgtgtggtggtaaacttcccttttcaaggaagggattactttataaagagtcaaaagtttaaaatgaaaataatctgaaaag |
| | caattcctcactttctcacaaactctgagactgagtccttagaaatcagttttttgctttgttctttaagaagtaaatgatagga |
| | agagggctgagtaattgaaatgagggaagtgatcttattaaatagattttgaatcccaattatacttaaattgtgtttctgtag |
| | ccaaaaatgtaataaacacgaataaatcttctttatcatcatttataataccatctgtacagataatctaataacctctatggaa |
| | tgtctgctctgttctaggcactctacatttctttaacttaatttcacaaccttacaaggtagttaaagatgagtatcacagagg |
| | cattaactgatactagaaaggttaagtgaagtgcccaaggtcacagagctagtttggatctgtgttcccacccaaatcttgt |
| | gccatattttaacgaggatttcacccaaggattctcttgctctaaacccacattctttcttaggccaagtacacatcaaagca |
| | ttattctattatttaagtgtcagaataatgaaaaaaaaacaaaccagcatttcttagagaagaaaggtaatgtgggctgagg |
| | ctgtcaaggacaggatcagaaagaggtgacttttaagccagcccttaggaggagatggagagagggtagagatgtttt |
| | gccataaagaatagcatggctatcattgataggagctgaattatgagaacacatggacacagtgtggggaacaacaca |
| | cactggagcctgctggaagtgggtaggatgaggggaaggagagcatcagggaaaatagctaatggatggtgaactta |
| | atacctaggttatgggatgatctgtgcagcaaagcatcatgacacacatttacctatgcaacaaacctgtacatcttgcac |
| | atgtaccctgagattaaaattaaagttgaagaaaaaaaaacgaatagtatggcataaggaaataaatagtctcatagagc |
| | attcagtctgggcctcataagagattgctttttttttgcttcatctctttgggacaaatgttcctgcctgatttaatagaaatggt |
| | atattctctgcagatacctttgagccatactctgagatgcagagatattttatgttggaaccagctctgtcagcatgcttgaat |
| | aggcagaatttttcctctaagctatctgaaatggagctgagttaccaataagcaaatcataacacacaagaatggaaaag |
| | acgaacatttattccatccaactagtgcttacattaatctgattaattaaagcatgaatgaaaactatttacttgtgagtgttgt |
| | gaaagagcagcattaaattagcccttcaccctagcagacacactgtgattgttggctctcattctgtttttagcctcattgctt |
| | tcttgtatttcctactagtggaagctgggccagtgcatagccagtgaaggctttctcttatttcactggtgaccagactcaaa |
| | aaaagcaaggtttttatattgaaattcgtcaagaaacttgccatcagacgagtacatatgcatgaccacaaggtcactacc |
| | caaatggaggcaagaaaatgagagttatcttttgcctttttgaagatttttccagaggggtttagttgccagttgggttttatc |
| | taatttagtttcctctaaatatttttataaacagcaagtttatttccagttttctattgtttgcattaactagggggtttaataatttct |
| | cacatttatcagtggaaataaactatttttattgaatatattctagcaaaagatgctttgaagttttttttggaaatatttaagaac |
| | ctaaagaatgtgaccaaaaattcctcataaaatagccttttgctcagggtcttatgttggcttggattctggctttgaccttgc |
| | aatctctgtctatagtgaccacattaaatgttacttggacttaataagatcttctgccttcaaagatgaagcaaaaagtgttga |
| | tagtttttaaaataaagaaatgaaagcaatagggtgcgagaagtgtcttaaaattgaaataaactcaaaataaaattgaatt |
| | tgtaagtaactggttatgacatggagtgccataaaactgaaaatatttgactgtagagatttcttggtaaagaagagaaata |
| | ttggagtcataattttaaatataatgtcaagttctgtttaaaaaacaaagtttatttaaaatttagacaagatagaaggccag |
| | agaaagaaagggtagctttatttagtaagtcaaaatatatgttatagcatttatcaaattcataagatatttgaccaagtgata |
| | aatatgaagcttatgttctgaaagggtagacaaaaaatgcaaccagctttgtggtagacagttgtttaagtatattgataata |
| | tcctttttaaaatttgtccacctgaagcctctcttgatcacctccttgcctccaaccttggaaattttgttagctctgcccctaat |
| | acttggctctctctattacatctttaatacttggctgtattaggaagtatattccactcagtggtaatcttggttttgtctccttctt |
| | atctattagaaggtgagcaacttgaagcagaggcttcgctttgtctttgtgtctctagcaccttgccaaaggcctgctaata |
| | aataattatcatattgaatgggtctacacaatgttctcagtatgctcagacatctgtgatcacctttacttacagagacaggg |
| | cagatttgaaatatttgcccctatctttgaagctaatacctggaggtgcaaactaggtggagcagagcccaggagctattt |
| | ctaggaagaaagcatttctctgatcctttgcttctgcccacagtgttaataacattttgttctgcacccacactctcaatttcat |
| | tccagctgggactcttctgattagcatcctgctctttgccttcccagagacagaataaaaggagaaggagccacaataac |
| | agaattagacaaattgtaacggtgtcttatgtcaatgtcaaagggagaaaatataaacaagttcaaaacttgtcttgggata |
| | ggttttttttttttttttttttttttgaggtggagtttcgctcttgttgcccaggctggagtgcagtggcatgacctcggctcactg |
| | caacctccgcctcccatgttcaagcgattcttctacttcagcctcccgagtagctgggattacaggcacccgccaccatg |
| | cccggctaatttttgcattttcagtggaggctgggtttcaccatgttggttaggctgggtttcaccaggttgggtttcaccaa |
| | gttggtttcaccatgttggttgagataagtctcgaactccttacctcaggtgatcctcctgcctcggcctccaaagtgctgg |
| | gattataggcatgagccaccgtgcctggcctcaggagagttattaagcatagcctgccaataacttcaaaataactttgta |
| | ttagtttgtgatttcagaaacaccttcacaaaccttttttttttaaatttgattgatccttatagcaacattgtcagtttgggtggg |
| | cgagggagttataatgctgtatccacacgaaggaatctgctttagttggttggtaaagtcttcacaggacctggaatttaca |
| | agattggtagcttgatatacagaagatattggaaagggaaaaatgttaccaatagagataacaatgtgagaatgaaaca |
| | gaggcataaaaatgtatgttataattcagaatactcagtagtacagaaactaagggtacatggtggagaggggatggaa |
| | cctacaaaggtaaagcagccaggaatcacatcagaaaggatcttgcttgccatgctaaggggcttggacttcatccttg |
| | gacagtagtgagtcactgaatgctgtttaacagaagacagctgtgactatttctgtgcttcataaaaattattcagaagcatt |
| | gttgctcatgattagaacagaagcatgccctttttttctgcttcaaaatatattctattcacacttgaaattcaattgcatgatcc |
| | tacccaaggtgaagggcaattaccaacctgttcattgtaaatctcactcttctccaaagaaagagtctccagatgaaagtg |
| | agggggacctttgctattacagggataatctcagatacatttaaattttattaaaaatcatttgcaactattagcccattatcat |
| | tattagtaaattaaatatgatattctttatgtttaaaacactgtgtctgatcagaaagaaatgacagtggaagaaggaaggc |
| | agaagaacaatcagagcactaggtggagaaccaggagagagacctgacttattggggcatggcagagaacctgagc |
| | tttgttcctgtaaccacaactgctactgaatttatcccaacagcaaaactcccactcttatattttaagccttctctgctggtac |
| | cctgttctaaacttacaaggctggatgactcctaacaagtgttaaaaatacttgactgtagcctagggacatattttttttaat |
| | gtgtgggtaaagagttttccttcttcaacaagttcaattgaagaaagcttcttatgcctatttcaaaacacacgcacaaattta |
| | ggagcagtgagatgaattctcttgcagctattttgatttgagttgtttcccccttggaatttgtggatagaaactgttatattag |
| | gctccacatcattagttatatttcaacttgaactttaaaagtagggctggagacctgaggtaatgattacccccaagaatcc |
| | ttttatccttttaagggcagatgaggtagatgagtccctacttttgtaactagaagaacttgataggctaatgtttctgatgat |
| | atcttctgttaaaaaaaaaaactaaccctgagatactctttagaaagagttctgttgtccaatacttattttaaattattcttataa |
| | aaagctctctattaaagaatcataatacacatcagtgatttaaatatcttaagaaatccttcagtaacaaaattaatctgtttaa |
| | cattttattgcttgccctctacttcctatcttcctataatgggtcagaatgtggacatgaatgtcagccaacctcaaatatgag |
| | gccgaggaaaatgccctaagagcgaacacaatagcaaaatgggtccctgtccacctcgcaaagcatagcctgcctttc |
| | accctagacttttatggggagatcattgtttgtcctgttttagtgagggtttttaagtttcatttgttgtaatatctcagcttataca |
| | ctcttatactcaacatacttaaactattttgaccacagaaattgtcttaacggttttattggaaaatctgctatcaataacatctt |
| | gaagactacatttctaaaaaagatattttaggcaacactgggcataccaaaggattctttaattctgatttcttgattaacaat |
| | ccttgctctgtgcatggcttgttccatgattttcctattctctatttcctggttggcacttctttaattgctgacacaggacccat |
| | atttcacctatctgatggctcctgagaaccctccactgggtcctctggacctggccaggatacaggggataaaagagtat |
| | gtcgaagattacttgagaagtctggggcaccattatgcagcaggtgaacatcactcttgcttaatactgtattggttgtaag |
| | tcagtcatatggtcctattaactgccagggaactgggagatgtagtttagttgcttgttagggaggaaggtgaaatcattc |
| | gatgaacacagagagttgtctctgctctccttgaaaaatatcagagattcccatctttaattcaactctttctagttgttttctct |
| | tttggaaatacattaatgcttcttcaaggagccaggcaaagcaatcagatcactcactgtaatccttatgaggaagtgtata |
| | tccttttcatggtcaaggttgttttgtttagtggaaagaaagttggttgaagaataatgtggtcagatttctagttctctctcttg |
| | aagttatagcttttttttttttttttttttggtgagacggagtctcgctctgtcaccaaggctggagtgcagtggtgcgatctcg |
| | gctcactacaagctctgcctcccaagtagctgtgaccacaggcacccaccaccatgcccggctaatttgtttgtatttttttt |
| | agtagagacggggtttcaccgtgttgccaggatggtctcgatctcctgacctcatgatcctcccgcctcggcctcccaaa |
| | gtgctgggattataggtgtgagccaacgtgcccggcccctacattatagctttcttaagtctctattttagtcagggttctcc |
| | agagaaacagaaccaataagatagatagatagatagatagatagatagatagatagatagatagatagatgatggatgg |
| | atggatagatagatagatagatagatagatagatagatagacagacagactatatgggatttgttagagaaattggcttaa |
| | ggagttatggaggttaagaagacccaggatggaccatctgcaagctggagaaccagggaagctggtagcgtggatca |
| | acccaagtctgaaggtctcagaatctgggaaaccaatggtgtaattctcagcctgaagtcaaagtcctgagaactaagg |
| | gggccactggtgcaagtctaggagtccgaaggccatagaacctggagttctgatacacaaggacaggagaagaggg |
| | atgaccctcttctttttgttctctctgagtctcagccaattgaatggtgctcaccaacactaggtgagggtggatcttgcttcc |
| | ttattcaatccagcaagtccaatgctaatctcttccacaagtgacctcacagacacgcaaagaattaatactttgccggcta |
| | tctgagtactccttaatccagtcaagtggacacctaaaatcaatcatcactgtcacctaccttttctgaggatcaatctcctg |
| | atatatagaataagagagaagtttacatattcactctgatcatagtatcttgttatttaagagccaaagtggatgtaagaacc |
| | agctgtccctacatcagactctgtatcagaagccatttgacaacacctctgacaggtgttcaaattctagtagaattcagag |
| | aaatatctgtctgccaaggcagctctatttattgccatcagttatcattaagtcctttctagtaatgagcaaaaatgtaatggt |
| | aaaatcagctagagcttagagatttgagtcctccttcttcacctggtcacatgtgagtcaactggcagtcctcattattcatc |
| | tttccactaaattttaccatttatctttataatttttcagatattatcctgcattgttcagcaatctcatcttcaagtgtttgtttctc |
| | tttccatccactcttccaaaaactctgtcccatcatcttggggaacattgaaagtcactgctggatatttgacatctggtgacag |
| | ctttatcttctgtatgggtgaaatttggatttcaacattgatatcttttattcttgactcaccgttccatctactccacttgtacatg |
| | ataatgtggcttcaatccctgctataaacatggcttaggatgctgcttctgcatattttgccccttcttgctccatagccacat |
| | atgaaaacttgtagcttggaccctagactccttttatccaaaaaaggagatgggttagatttccaaatatgcaggcatttcc |
| | ccacaggtcttctgcatacagaaaagataaagataaaaatggaaacttaaactaacctctggcaattcaatgggactctct |
| | tgtgtacatgatcacaagcatctgaaaaatgctagatgaggtattagatctgggagatactggttttatcaagcatatcttgt |
| | catttccaaggttttctagttactgacaatgagtcactgctaatttttccatcctttttgtcacaattactgacctcaagaaggc |
| | aaagagtttcatgtgttctttgaatggaattgaagccttgtgcatccaaagattctgttgacttccaccctgcaaactgacttt |
| | gatgtaccaatgactccagcaggctgggaagatattacttccccaggaaaactctgccccatttctgtttatgcaagtagc |
| | tgtccttgctgaggtagtaggatcaactaaacaacaaaatcttctttgaggcctgagcactttcctttggggccaccttcttg |
| | agttcagcctgcatcactcactaaccttggttggtcacaggaacagttatgacagtacatctgcacagatgttgaaggcat |
| | gactagcttaagggccttctatgtcccttcctttaccactaaccttaggatagagtttatttctgatagttccagcctggtggt |
| | ggtattctactcctttgcagaacacatttaagctactactctgatttcaattctcctctctacttcagcatctcagtgatgaacta |
| | cattcttaaaacaatgttagtccacccagaggattctgtattgctagtcaatttttagaacaaggagatactcactggagtca |
| | aacccaagggaagaacaattaggctatatatatatatgggtatgtgtgtatatatattgtgtatatatatatatgtatttgatag |
| | atttatgtttatttttacatacatgttaagtacaaatttggaagcataagatgacttctctgcaattattttgctatgtgttgctttcc |
| | ctttggaatctgtggatagaaactattatattaggttccacatcactagttatatttcaactaaaatttcataactggagctggg |
| | tatatatgcgtctctacttgatatagctgcatcatccaatgatgggtatatatgcatcatccagtgatgctgggtgctggaag |
| | atcgccatttagtacccaggtggcaattaattttgtggtattgtatttctgggtgttccctccattcctgacattctttatcatcta |
| | tgattcaattatttgcctggctcaagaaagcacctttatttcccagtgaaccacgatgtgatagaggtgactttaagtaactg |
| | gtttattgtgatcaacatgggtcaaaggaataggtgtgcagtgccactccagaaacttaccagagagtacttttgtcttcta |
| | gaaagtgaacctggaggctcagtttctccagaattcatcttcctctgcccactccaccaacagaacagttggcatccttta |
| | gaccaggggtcccaatcccaggctgcagactaatatggtctatggcttaggctccatcattctagggtttgatttttagttta |
| | cttttcttgtctgccatactggaatttgctaatatagctgtggtccctgtaccttattatggtgtaacaaatgcaataaggatag |
| | gggcttcttctgataattaagacttccatctgccctccgtaggttcaactttaatgcttcctacagatgggcaacagggctat |
| | ctgctcagtgcaacccccagaattccaattgtctgtgctgtctccactccataccataactaagcctaggaagcgcctcct |
| | gaagttcttcactgggctgaattaagaacaggatcatcgttctctatctgggtgttttctaccccactgagagaggtgaaat |
| | tgtcagaacaaaaatttaggaatttaccaaatgcagtttttttttggctgataattactattattaacaataatgacaatgataa |
| | gcttagaaaaattaaacactaaacaatggctgccgtaagaacactgaaatgactttatagtttacaatcagtctttcgtcttt |
| | cagggatatttttgtggacatctggtgccacattttagtatggggttcagagaatatcaatttacctgtttcttcatcatgctat |
| | attgagttatttactccacaataaaggtagacctactgatgagaaatgtcagggcaacctttgtgtatttcagttaccctcag |
| | caaggtgctaccagctagcattagtgtatttctggtgttggtaacaggatcatgtgagccttgtagatgagaactccttgtt |
| | gctcaggggatcatggaatgctgctctctttcagacagccatcttcggggaagagaagggtctctggcagagctctatg |
| | ggtcctgcacttctctttcacctctctgccttgttctttcactcattaatttcttgacaatttattttccttaataactctagttccttc |
| | tgtttatcttcttctgtcaatccttgcatccattgcaaatccatcacctgctctgaaataccacactgaagggaggtactccta |
| | aagaccttccttcttttccactcttgttttacccttggcttacacaactgttgaacaactggagctgaggacaaaaagcacat |
| | aaattccaaaggtcaacggaatcacttcttcatagtttacatctcctgtgtcttaagtagaactcaaatagaacataagcatt |
| | gagaacagaggtctgagttggaggtcaagacatcgtttactagctgagggactttggctttatttttctcatctctaagagg |
| | gaaaagataaaaatctacttcacagacctgttgctgggaataatgagataatttatgtacagtacttagcacattgcttggc |
| | acataaaaagtgctcaataaatgttatatatttataacattatttgtaacacctcgtctttcaaggaattatgggaattattctgtt |
| | tgtcagactttgtttatggaaattatgtccttttgatggcccaatttttcttttccttttcttcatcttttgataacatactattaaat |
| | gtcaacaattcatactaattggaactagtgtaataagctgtcctcctcatcagtattagcagtctttgaacatggcataaaaata |
| | tgcccattttggcttacaggtccttgattctctaatgataatagtgcaggaaaaacattagatatggagttcaactttttaaaa |
| | ctgtcttgatgttggtgaccaataatttaaaagaggttagcatattttaaggtgactaaaactgtacaaatgaaattgccattt |
| | aaaaatgtctgttcatttgcttttctccaatgcagatattttaaataagaagaagtgaggcaagaaaaatgtaaacaaatatt |
| | aggtataaactaggaacttagtcttttattttctgaatcagatggatgatatttaaaagtctgggtgaatgataaatggtagaa |
| | aaaaattaaataacagtcaatatgcacaacttaatggaggtaaaataagacttacttacttttatctctataaattaaaatgatt |
| | tttgaaaatcaagctgccaaaataaaggtgtggattttatcattaaagcaatgcccaagagacatgttattgagaaattatgt |
| | ggcattggtttgttgttactagcgaaatgaatgcctaactgaagttgatttcataaagatttagtgtgtgtatgtacttgtgtgt |
| | ctgtgtgtatatatgtatatatatatttgtatacacacgcacacatgtctaccatgtgacatgttgctatttttacaacttattttaa |
| | aacagctaaaattttctagatgagattaaacattttccataaaaataaaattacaatagttacctcaaagtttgtttatcctgga |
| | aaaaaaagtgctaacataagagttgttcaataatattttaccaaaattttgtcagaactccattaaaaacaattcaagaccac |
| | agtggtctaatttcaacactggcatcattaacctaacatatttatggaatattcactatatgttctgttttctgcttaagtatatga |
| | attaaacatagtttaaatagtttactcaaagaacatgccatttgcttcattgatttcacactctctgacgtatgtattaataagaa |
| | cataggaatccacacacagaaataatagcattcctatggtgcattaataaaaatgtatgaattttctatgtctaggacatgta |
| | ttcattatttgggtgggtcttatatttcaggtactgttctgtgaaccacgtttatggaagtgatcaaaacaggcaaacatctct |
| | gccttcatggaatttacatttttcttggaggaggcagacaatagacaaaatatttagtatgtcagctaaatactaaagggaa |
| | aaaccagaggaggggatagtgtgtggtattgagggtcaaggccagtctcagtaagaaggcagctgtgagctgtgaca |
| | agaatgagggaagtgagccatacggagcccatgtaagagatttccaggcaaagagattaaaaggtggacgcctccta |
| | aggaatggacatacctgccatgtttgagtaatgagaaagaagataaaatggctaaagtagagtgagtgagggcaagaa |
| | taacatcagagaagtaaaggagtctagattgtgagcattgtgaacactggggaaggcatagactattcacaaggagatg |
| | ggtagctactaaaaagttttgagaagaagagtatgtgatatgatttagggttgaaaaggattgctccagtttctgtgtcatat |
| | aaccctatggtcttgaaactgggatattgtatccctgggggcatgaggtggcgtgcctagaggtacacaaaatcccagg |
| | atcagttagtcacatcttccctaaggataaaatttaccctcaagactatggggaaaaatgattactgtgttaagaaaaacatt |
| | taaattttatataaattattgtaaaattcccgtgattttaaaaataaagtatcaataatatttcatatttgtggcatcttaggcttta |
| | atcttacaggtttaagcttcttggccatcaggagtaatttgttgaaattgcaaattaatttggcaaaattatcataattatgtgc |
| | cctgagagcttatgtatcttatgatagtgcaggaaacttgtgagatgaaatagttgagtgtatacattaaaataatttgatcta |
| | ccaagtcactttaattatttatttgttcagtgttaaaaatatattttcataaccaaactaatacatcttaataatgttgtagaaatg |
| | gaagtaaggtaaaatttaagaaactgggtttgttcttctgacttgcttattaatactggataagttactgaagagtgggctttg |
| | gtttcctcgtctatttaatagtgtgggaacaaccttatgttaaagcaatctattcagtataactttagcacacagaaaataaaa |
| | tattatgaatccaaagaaagtgtagcttgcaatcatgtattttattaaatgtatttatcagcattttggcttctgatggacatgtg |
| | tatatacatattttgtggataaaaaaattacttaactatttttatattagatactatcaattattctggctgatctggggacttttaa |
| | tggtaaattttatatcaaatctgacaaagacagtatttcagacccgtttaagacctagaactcaccatgagttctaaaattgg |
| | ttctcagcaccatggacagcgttactgcaataggaaattaaagatcgatttggccccaaattaaaatggtgttgtaaaaaa |
| | ggggagaaaaaaaatgcctatccttttacttcaaattttaaaaaaatgatcctggccttcacaactgttcataagaagaata |
| | attaattaaacaaacatatattgagaacatcatatgctcagtaaaactttgattctataaatggtgtcatttaccaaatggattc |
| | ttttgacaatttaattttctcttatctctctaagaagatgtaactacacactatagtatactactacaattatcaaatttcatgttgc |
| | atgtaacttgtcgtctgtatttttgtagttagattagattaactaaagatttttcaagtttgcctttaagtcatttaattttcctgcctt |
| | atctttaacctttcaacattcctccaaacaatagcaacacaagtgttatgtgttaacttctctagtgacaaaaacttatacttct |
| | ccacaaagagatgtgatgttcattatcaataagcttgacatctaaaattgttttataggagatacatattactttttcagatggt |
| | atataaagttaaataaatcttaagttttcaatgatgggaaaagcttccatttagtttaaacataatgtaaagaaatttgaatccc |
| | caaaatagaattataattctaaaaattcatactataattcttcttaaatgtttaaattacagttaattaaagtagttgatttcaaata |
| | gagtggaattatgggctgtacatcatttaattttatgtgctgactgctacatagccaaaggaacgtgaattaagatggttcc |
| | acttttgaccaggagatggagctgtcatgtaagatgctgcctttatttatttatttttctaatttagcatgctgttttctaacagac |
| | attgggtaccatcgaatgactgtcagaacagaaagctaaggcaaaggagggaggatgctgtggtcatcctttcttgttttt |
| | ttcttctttaatgaggatagagcacatgtgagattttactttctactccagtaaaaattctgaagaattgcattggagactgtta |
| | tattcaacacatacgtggattctgtgttatgatttacatttttctttatttcaggtaagccagcatgattctatttttgacttatcca |
| | cggattgttatctatgttaagaatgacatttaatataagatgtgtgctttgttagcttgtattcagatctaagagattcaaaagc |
| | tctaattctagctgttgtgcaatttaaaatcttcctaggctgaaatgagctctgactatgacataccgtgttttattatttctttgg |
| | ctttctagctgttggtctctgtctctggctgtatttgtttaccttttaaaggtaaagctttcaaagtgaaggatgggttttcctttc |
| | acaagggaaatactgctccttaatgcagtgaacaaattgtagagatataatgaagcagattaacatggcagtggctctgc |
| | caaagctcagggctgagccttctgattgcagcctcagaggcaagccaatattctgagtccaaaggccttttgaacgattt |
| | ctgctggctatgatggtacagattgtttatgtctttatttttacctcttagaaataatacgtaagtcatttctcccacatgaatca |
| | agatttatgtaatgtgtttaatagacttggaatgagactatagaccctccccctcccccagtatggatgtgaggagtatgct |
| | gaacacaaattaattgccatgaaaattgattttttctgtattttattggatttgggataatagaaccgtggccattttaaatagtt |
| | gatattatagaaggtgctcacattttaaattattttaggtcatgtgacattatctgtgttaaagagtggaatgactcaggaggt |
| | ggaagttgcatgcaacagccttgcttctctctgctgcttctcgtgatttcccttaaatccttgcttgattattattagaataaca |
| | ggaataaagacatatagaaatcctatatttaaaaaccactgattttcatttagagaaatttaaagagattttacaaatattttaa |
| | aaatagttttgtaagattgtttttaaatgcctcacttctttctcctgtggaaagagctgtgtgtgtgtgtgtgtgtgtgtgtgtgt |
| | gtgtgtgatggtggaggtggcaggcggtggaggtgcatcaggatgtttttatctcagtcattgttttcctgtaaagaggta |
| | gttttgccatgtcagaggttgtgaaagctatggggaatgattttataaaatggtttggacaaaccatgaaaaaaacatgtttt |
| | ttaaatgtgtggaatctatagagtttgtagttctacatggctccatatacatggagatttttagaattacagatatttaaccattt |
| | atccccattatcatttaaagaaaagcaattgtaattgagagataaggtggatttcccagagtcacgtactaaattacaataa |
| | ggcaaggctagaacagtgattgacagttccacatcttccatagcagtaacacaattcacctctagtgtgaacatatcagg |
| | atggcatagaccaggtatgatgatattcactgtgcgtgtgtgtgtgtgtgtgtgtgtgcatgtgtgtgtgtgtttggttatattc |
| | tgtaaaatagagtttctttcagtatactgttgctcaatggcatattatatgcagtgctcaattttcttaggtttcttgtcaaagcat |
| | ccaagaaaactgaatgacaactatggaaactctgtttcttagtatgcctttttcattattaggtggtgtatactcttctaccctat |
| | gctcatgaatgatgtggatggccacatggctttccctcctacctaatagaaaagattgtacatataggttaatctattgacaa |
| | ctataagaatatcatctcatgtccaagcctttcctcctttgttcacttactaggcacaggctttaccctacttcttcaatatcttg |
| | agaaaagaaagggattaaaatgtgtcatgtctacatatatttgtggaataccacgtaccaaccaagtgtccatggtagag |
| | ataaagaaataattagtcaaggagtttacctcagggaggttaacatgcaaatatacatgtgcagataccactatatctcaa |
| | aataaggaatgaagaaagatgagaaagtgaatgggacaggaatatcagcacctatcttgtgccaggcaccatgttacat |
| | cctgggattcagaacaaaacaataatgttaatttctgtccatggggggctcagaggaaaagattttaggaggatgtgaca |
| | gttaaattagatctttaagagtgagttggatataaataaagaaaaacagaaagttgcttattccaggtacataggtgaggc |
| | aagtaaatacaagaaaatggagaactataggcatcgtatagggaaactgtagagaaaggtaaataacccaatctgtttg |
| | aagcttttgttgtcggtgttgttgttgcagttgttattttttttcccctggggcagtaaaagtaaggataatgaatttggcttttg |
| | gtgttatgcatttgacatactgacacatactcatacactggggtgttttataaggagctgaacatgttatatccatgtatcaga |
| | ttagtggttaaggctgaatgtatagatctgcatgcagttgtgggacaaaattaggtcatacataattaaacattaatgggga |
| | acaaaagagaagtgaagatgactgggggcataatcttaagattaggagagtaaaaaagtgtagaaaactattattaaatt |
| | gtgtgaaaataatgtgagaagttgacggaataacaatatcataggaggccaagcaggtaaattgaatatcaaaataggg |
| | atcagaaacaatagaaaacccataaaggggttggttcataagaggatagagtaaagatgatggatttagttatcattatta |
| | ggtcataaattaattttaagagaagaggtttagttgaatggcagagacaggggctctgaggactaaattggacatgagaa |
| | atctggggagtatatttaagttttaagaagtttgccagggaaaggtaggagagataacattttatacctggagaaagaag |
| | gtactgttgagggaaatttaactttagtgcagattagaaaaacctaagccctttgtagagtaaggtatgaagtcagtagag |
| | agaaaattgaggaaagaagaaacaaaagtacaccaaggtcgtggaagagacagaaggaaggtttgaaaatgcttttc |
| | cttgacacaggaagaaagggtggttgatgataatgagcaaagctggtgtaactcactttgggtggcctgtttttcgaaatt |
| | aaatccttgtttgtaagatgaagtatctcctctgattaaatcagtttgttcctcagtaaaggctattggtggcatatggactttc |
| | tggaagaaataattaatcctcagtagcaaaacactttcctgcagaatatattttagtcacattaattcttgaattggagactaa |
| | taacactgtttcacatgaagcttttacatacttcagttatcaatttcttattcataaccacctatttaaaaacaggttctatttaaa |
| | acttgtattgaattaggccaacacatatctattatacatatctagtttgattactaagggctggtttgccagtatctaaattaatt |
| | attcaagttctttccttttttgaatccttcagtttcattagcacgttcaccaggacttgtgtagagagagtcaatgtcatggccg |
| | ggtgcagtgacttatgcctttactcccagcactttgggaggccgaggtgggcggattgcctgaggtctggagtttgaga |
| | ccagcctggacaacatgctgaaaccctgtctctactaaaaataaaaaattagctgggtgtggtggcaggcacctgtagtc |
| | ccagctactcgggaggctgaggcaggagaatcgcttgaacctgggaggcggaggttgcagtgagctgagattgcgc |
| | cactgcactccagtgtgggtgacaaagtgagactctgttaaaaaaaaaaaaaaaaagtcaatgtcaggaagctcctttcc |
| | cttagggaactagcttccttatgaagcttcctaccagaagttccttttttctccatttcttctggcagttacagaatcctcttgg |
| | ggctttcttagagccaatctcctctaaggtgaatgcattttcttgcattcacctgtcatgaaatggcagtggaaagacttgaa |
| | gaaggcacaggcgttggtgagtcatatatatattttgctgataataatgaataccattggcaatataatagtggccattgaa |
| | atattatttctattaggaaaagataacttgcatcaagaatatgagatgaaattctattaaaaaatgaaaagtagatgggcca |
| | ccgtggctcttgcttataatccacttcgggaggtcgaggtaggcagatcacctgaggtcaggagttctagacaagcctg |
| | gccaatatggtgaaaacctgtctctagtaaaaatacaaaaattggccaggtgtggtggctgacgcctgtaatcccagcta |
| | ctccagaggccggggcaggagaatcacttaaacccaggaggcggaggttgcagtggactgagatcgtgccactgca |
| | ctccagcttgggcgatagagggaaactctgagtcaaaaataaataaataaataaatagaaaaggaaaggaaagaaga |
| | aaaataaaagctgacctctctataacactcatacgttagtgttatctcctctttatccatttgggtttagtgaaattggaggtca |
| | gtggaaggagaggtagaagtggattttgcaaatcagaagtataatttttttttctggtaatgttccattaaacttttcagtcaa |
| | aacctttatgtggaagaagacataattactaactacattaataataataatgaaaaaatcttatcatgtaatatcgttttgtttat |
| | tagtgaaacattttagagaaatgatttaagaatcaaaggtatttaagtatattttatttattggaatgtgataaaatatttttgag |
| | ggtaaaaaactataatttcagttaactgtttgtacttactaaaataacatttagggggataaaatgatcttaggaagaaattg |
| | cccacaaaaagtgttttgaattgtttagattatttttatagttttaagttgttaagtcttttgctggtatctaggattagttgggagt |
| | ataatatgctttgaacatgaacatgtcttggtgttattagcaaataggtatacaattatgtgattgtgtaattactcagctacat |
| | cttgtgttgtgtgatatgtatggggaaatgacaacaaaataacaacagttctcttaagcatatatgaaggtagtgcttgaaat |
| | atcatgaaaaaaagaaagggagattatgataatgttatgataagagaagactgacactatccctcttctctgaataactgat |
| | atcaaaatgtgaaattctaaggaatgaagcaagctggtcagggcaatggtgatgctgtctctgtatcagcccagcatttcc |
| | agctaggatattttccttcttttctgtgtgatacagtttggctctgtatcctcacccaaatctcaccttgaattgtagttcccata |
| | atctccacgtgttgtgggagggaccctgtgggagataatttaatcacgggggtggttaccctcctgctgttcttgtgatagt |
| | gagtgagttctcactcgatctgatggttttataggggtttttctccctttgcttgacacttctctgtcctgacaccttgagaaga |
| | aggatgtgtttgcttacccttccgccatgattgtaaatttcctgaggccttcccagccatgcagcactgtgagtcaattaaa |
| | cctctttcctttataaattacccattcttgggtatttcttcacagcagcatgagaactgactaacacactatgtctcccctggat |
| | tgagcaaatgagggagcccagcaattgtcaggcagctaggatctgtgaggagtgcttagacctacacttactatttcctg |
| | aattaacagtctagaagacaggagcctagttatacagacagggtgaccataaaatgtgtcatctaaaccaggattcagttt |
| | gttaataatttctccacaacgacaggtataaatcaagatggttccaggtaaactgggatgcacagtcactccacttctaag |
| | ggtcaagaaacttctacatttaatcagtctattcaattaaattgttcatgattttgttgcctaagtgttagaaagttaaaatgtga |
| | aatgacctatcaattagacaaagggcattcagctaagctgctccaattactggccttgataaatccatcaaccgaaatctct |
| | aatgattcttgttgaatccttagttatatcaccaggaatgatgtcactccaataatgttaagcctgacttcccagggaattcaa |
| | ctgtgtgcaaaaaaaaactgttagtaaattatgctgttattacaaacctagactcacagcatcataaatctattcaaatataa |
| | aagcaaaagcaggataaaatatggattcaaattatttccacatttgggtatgcatcttttaaggagttttcatgaatgtgtaat |
| | ccttagaagataggaatttagctccaagaacactggaattttctccactggcactttatacccaaatcttggtaatgtcaata |
| | tgcatatgtatggcctcttaacagactatcctgtaaagtagttaaattattttcctccaatgatcatgttttttttacccttcttcag |
| | acatccactcccatggttttatcttgaaactcttcatggccagtaactgtggcctgcccataatccttccttcatacatcctac |
| | accacctttctagcgcatttcatctacaactccaattcattcctttaatcccaccaggcctctaaatcactgatctcgtcatact |
| | ttgattatctctcactaaatgtcctctttcctttcattagataaattccacagttattcataaaagtcactccttgtatatacataca |
| | tacctcacaaactcctccgctcagtgtttttttgtttggttggttttttggtaactgtctggctaaatcttcatcctcatattcagtt |
| | cccaccttacttcctgcctgcacttttgcagctggacgcatgcaaaagtgggtccttcatgatgcccagcaatcatgccac |
| | atgtccctagattgatgcccagcaaccatgccacatgtccctggatcacttactctcccaggttcttgtacttagatttcata |
| | ccctttcttttctcctcaaaccaccaatacctcctaccttgacctcactctcagttgacaactttgtttcctaaatcagtgagaa |
| | aattgaaacaatcaggagagatttttccccacaggtttctatcactgcatgtgccttcctaccaggttctgcacccacataa |
| | cctgacctcccacccattaccttaaatcaactatccctgtttctatctaaaatcattcctgcatctgtgcactagatcctattctt |
| | tcttacttacacaagggcatctctagagcattttatttctctgtctctcctgcattagtaattttagctctttattatatttgcataca |
| | agcattttattattgctcttgtatttgaatttttttacttccttcaactatcttttttttattttttgttgtctttgcaataatactta |
| | atgaaagtgtttgctgtattctctgtatctatgactctctttctattctcttttaaatttacgttagtcaagtttttactcttaccatt |
| | tcaccaaatcagctcctgatagaaatctgatgaccaccctattgccaaattcatcagtccatcctcagctttcatcttacttgaccatcc |
| | agcaatattggagacagttcattgctgtctcccccttgatacactgccttccgttggatttcaggatagcacacaattttgac |
| | tcttctcttagcttattgttcactccttcacagtcactttactctttactctgccttcccagacttcattatttgggtgtccctgattc |
| | tgtccctgattttcttttcttctttatctactttcttttgatcccattgagttttatggcgttaaatgccatataaatcatgatgacatc |
| | caaatatttacctccagttttctgccaaactgaacttgtgtgtctaactgcttatttggcttggatatctagaagacactgaaa |
| | agttaacatccctaaactaaatttcggatagttcttcccaaccaacctgctccaatcctcgctttccccaccttacttttttcag |
| | gagataactctagttttcctcatcctcaggctaacaactctagtatcatccttgattattctttttctcttatatctgacattcagtc |
| | catgagaaattcctgttgatttcaacttcagaatatacccaggatctatgtgctgccaccttcacactgctaccctgctcaag |
| | ccaccatcaagtttctcatggatttctgccttaatttcctaattggactttattcttctacttctccttacaatctattctcaatacg |
| | gcaagacagaagtaagattacgccacatccctgctcaaaattctgcaagtgatatccagttttctcagggtaaaaggcac |
| | ctgacaatttccaaaaagtctctatttagcctggtctccatcccattacagtatttctctgaatttgtctccagtttttctttccctc |
| | attcactttcactatttcttgaatattgcaggaacacttcaatcctttgggctgtttcctctgcctagaaaccttttgcctcaggc |
| | acttgcatgactaacctcctcacctgtttaaaatctgagctcaaatgtcgtttctaaataatacctactcttaactatcccattta |
| | aaaatctcagcccccttcctctaacttcattcttatcgtctcccacattcttctatactttcttttctttgttatcatttgctttgcttt |
| | gttctttttttccttttctttcctttcttttatttccttcccttctaacacatcacattatagcatactgtatacattgcatttctctt |
| | gttggtaatttaagccccaaatgggcagagattatcatctgtttggtgcactgatgtttcacaaatgcttagaacacttcctgatctat |
| | agaagagactaaaaatgtttgtagaataaaataatgaatcttagaaacttaaatttattcctttaagcatactaaggggcacc |
| | atgctttctctttcactttattgggctgattcacatttacaaatgacccagtgcaatctgcctgatttttcattcagtatttacacc |
| | aatagaaataaaatgcatattagtgttataaaatcaaaccttaaatgacagtaaatgtatcattaatagttactgaagaccttt |
| | tcctgatatatatgacatttaatgtttttctcttgtgacttcattgttctttgtttgcttgttcatcttctagattgtgagctcctcaaa |
| | gtcaggaacttggtcatcatatctgtctcccaaataccacactcaatgtctggcacataagaagttttacaaatatgctgtttt |
| | agtcataggatgagcatacacttaaaatttaatactggtcctgcatatgctgtgtcctgagctttcgctatgtaggttgcttta |
| | attcttacaaccgtgtggagcaaagcactggtctcattttacagatgtgataaagctagttattttgtccaggacttaatcatt |
| | gcagtattctcactgtctaatctctagtgaccatatcacaggaagttaattatgctctttttttaaagataaattgaaataatgct |
| | aacaatgccagatttcttggaaatgttttgtttttagtttcagttagaagcacgccttagcttagtgaataagggacaatgaat |
| | gttatttattttcctgctcaattcagttgggaaaattgacctcatcgttttgtgttttcaatcccttaagtgtaataaaattatttact |
| | accttagtttatataaatcaccaagatctaatggaagccacccagttaactctcctaaagagtcttcaagcatgttttgttcta |
| | aatctcagcttaagtattttccattgtttaccacctaatgtctatactacttgatttatagaaagtcttatctaatataaatatgcta |
| | actagctatgataaaattgatcactttccacataatcatatactgaaaaaaaattccactgaatatatttctgccctccataga |
| | ttccaggatttcagtgaatttttcttaataatctctgcttcagaaatgtatataaacttttgtcctaatcaaatctaactcaaacgt |
| | cacttcctttataatgttctctctgttttcacatgcagcatatacttccattctactctgcacctatctctacacttgtaatgtgaa |
| | gtagccagatatatgtggacctgggtcatctgtcatatttaattgtgtactatataaggacagatattgttttgtatggttctatc |
| | aagctacccagtatctacatatactgccctccataggaggtaatacatatgctgagctctactcatttaaaacttcaatcaa |
| | ggtactattggaatttcttagttttacaacataactatgtcctacttcaagtgacctattaaaatagctctcaatagtttgctttaa |
| | tacaactgcctttaatagcttttaaatttagtgtttgtttgagtagactttatggtgatttgtaaaatgatatggatatagtatata |
| | gaaaattttaacagaaatgtaaataataattataatttagggaaacggtgccctcttgctctatttcacacacacacacacac |
| | acacacacacacacaggcacacatatacacacaaaaaaaatctctatttccaaatcttgttcacaaggagctctctaactc |
| | catgagtaatttttacaaccaataacagattaaccaacaatagtcacacgaacatgtacacacacatatacacagcctaac |
| | ttccccacatcattattatctatctatcgtcaagaatgcagcttataaaatacaatgacctggctgctataggaaaggataaa |
| | atgtaggttgcagtgatcttctgacattgagatggcaaggaatgtgttttttgcaggaatttcaaaacctgaattgtagaatg |
| | atttgagatactgccttaactaccaacaaatttgatcccatgttcttcatcatttttctgagattctggtttcccagatttctgacc |
| | ttaattgactcaaccaaaaaaatctgcttattattaaagaccataaggagtaatcaacagaacaaaatatgatttaatataat |
| | attaaatatattttacaactcaaaacattctaaaagatgtgatggtttctgactataattcagtatatactattttagttttagtga |
| | caatgtttctaacaaatacaggaaaacgtttcagaacctagtgtcctttacagcaagtagcaaagatggactcaggaatat |
| | gtaagcaaaatctagccaaaccaaaccaaccaaccagacaacaacaacaaagtgtgttgctgagtggctcatagacaa |
| | ggacaagtatttgtgatgttctaatataataggtggtaagaaccaatggaacatctgtgattcaagtgagagtcgtggtggt |
| | agaaaacaaaacaaaagaaacgataatatgtgtagaaataataaaagaaaaaagtcaaagggtgtatttttacaagtaca |
| | cgttcaaatatagcgtgatgatattttatctcaggaatttcatgcaaagtgctgcgtatgttgatatgttaggtctcttctggcc |
| | cagtgacgattatcttgtgtggtttaagttctctatgccatgagtgaggtaagggcatttattccccagatccttccctgcca |
| | gattactgtggttgaatgtaaacaccacaactcctgccaagtggcccctactccctctgattctaattattatttcctctttttg |
| | cctcctgctgctgctagccttggggtgcactctgctttgtttcttttccttaaccctaatatacctttgtaaataatccttttttgttt |
| | taatctctcaattaaaatctcctcttggggtgtgtcatctgttttctgctaagactttgattaatataatagctaacatttatgaaa |
| | tatttactataatacaggcactgttggaataattcacatggatccaatttttttatcctcacaacgatcatgtgaattaggtact |
| | attatgactaaatttaaaatataaggctactgaagaacatagattttaaataccttcatgatcatatagctaaaaactaagac |
| | aaaataaaatccaggagtctgactccagagcagagcttagatgtgaaagtaattaatatgcttttaaagaaaatcttatatt |
| | gttggtgtctgtataaatcccatagacaagatctaagtacgaaaaaaaaatagaagcaagtcaggaaaacttagttcaca |
| | ggtaataaaactagacacagatatctaaatttagaaatatcccatgcatatattctgcatatctgaatgcatcaagatattaat |
| | aagcatcttaatactttggaaaaagtatagaggggactttctgtcaacatttgaagaaaacatttaactagtataaatatatc |
| | atacaagttaactgaaaaatctatctgctgtctatcctcatgttcagttatcaagtatatatcatttaaatttaagatgaacaaa |
| | acactacttaaatcccaaagactcattaaagtaaaactagtaaatgcattttatttatcaattatttccctgaaaatgcacattc |
| | tttttaaattaattttttatttaaaaaagagcatcatgccattaaacaaaaaaaggaaaattttatttactgggaaattcttgctg |
| | atttttacatgggattgtgacaaaaaaaaggtcagatttttatttttattcacagtaaggagagattttatcttcagggatgatt |
| | acaaggtggggaaagggcaaggatctcaagagttaggcaaagatttctcttttagagagaggagtaaacaaggctaga |
| | atgaactggatggggagaagagggatttaagggtggtatgatctgacagagaatgttccactctgagaccagcctgcta |
| | ccttgagggactatgtaaggggagggggggtgtggctgcttcatcttgaaagtaagtcaaaattcagggatttggggaa |
| | agtagaaaatcttaaccaaaatttggttaataggcattttgttcccattggtcagtggggacgggcagtttagcttatcattta |
| | tgaggtaaagaatgggaattttgaagttctgtgttgggtctcatcacaggtgaatgggggacatttgtaagttttgcctgtg |
| | ctatatggggaagggtcattctttacagtaatttcctagaacatgaaaggataggggggttcttattatttcatctacataaa |
| | ctggaacagaagatgatttcactggtttattaaactcctcaggatcttctacctgcccttcttaacctttacattccaaattcac |
| | ttctatctcctttctagagctggtaatctttcttccatgaagtttatgaaattcttcatgacatgcatagactttgacagttctgttt |
| | ctgatgtgcataatacctaccagattgatttgtgataatatcattgagaggccacttatagcctatttatgactcagcaaacc |
| | atggtcttttacagcctcttttaagatggtttggagagataaagaataatacttcatgacatgtaaaaattatattaagtttgaa |
| | gtttaatgtccataaataagttttattgggacacagccatgcttatttttatataaattttccatgactgttttcttcctacaaggt |
| | gagaactgtatacttgcaacagagacaatatggcccacaaagccaaaattatttactaaatcatcccttgtagaaactaag |
| | ttgcagactctggtctatatagtcacattacactacaatgatcaagtatagatgtttgacagtatttaagtttctgaatgaatga |
| | acctatatagagaaatgtataggtataactatattactgatatatcctattaattttattatttttctgttgagtttttagcttttctag |
| | gtacataattttgtctgaaaataattaggacaattatgtcatgcctttctttgctaatatttctaagcaatcattcctttacttatttc |
| | taactaatgtttacccccagttatattgtcagattttcctggaaaatattaatagtgcaaatttcctgctgtttcctaactttgaca |
| | taaatatttatagtggctcttcactaaattacatttacatcaatatccttagtgatatattgatgtaaatttgatttgagacagatc |
| | aacttcataatgtaaggatatttctctttatgaagagaatttattataatttataaataaatgtgatatctgttagatgacttttaag |
| | catctagatgtagttcatattctgttttggcccattaatgtcatgtatttcattaatatatttttaagtactgcaccattttttcattac |
| | tattttggatcttattttggggcattatgttattaagtagttgtatgttttatttaagaagtttataactgtattcattaatgatattgg |
| | ctgtagatttcatttttgagggtgttctttcggaaaagtttactatcaaatttatatttgcttcaaaaatatttttgagtaattttcct |
| | atttttctatcctttgaatgtgtttacaaaatgtggaaataagttgtttctagataatatcaatatttatttgctagaaaataaaaa |
| | ctcacctgtaaacatgttttattgttattttaaagtggttttgcatatttcttttaactcatttccttattaatttcctgatcaagttt |
| | tcacttcttacatcattttgacaatttcactttcatagacgtacacattttgtagaaaaattcaaagattttaacatagttaataatttt |
| | gtttgcatacttttcatctctcaaaagctaagaaaaatgaattcatgtcttctacaattaacagctttgcataataacgtactac |
| | tattaactttatgattttatgaccagtatatatttattagaacatttacaagtacaaaattaccttacttatccatttaagttatttgt |
| | gttgagatatgttttctctctaataacattggaaaacctaaatttttgtttgtatttgttgatatatttttgtccattttttattttta |
| | attttggcaatctgttctatgcttctttcaaataaacatctgttcaacttttgctttgtgatctaatttggtactttgtgttggttaat |
| | tataaattaaccaattcaaaatttattattttaaatatcagataatatttgatttttcttctcataacttgttttgtggtttctactttt |
| | cttatatttttcttgattttcccataggggtatatcaactgcagtttttcccatctttattttttcttcttctattttttctagcttttt |
| | ggaatgcatgtattgccttatttcattccattattttaaaagaaaccatcagcctcataaatgaaactatctattaactacccttacata |
| | agataaaaatattgttttttctttcccttccaactattttccagtttaaaaaaatgtacatgatttggtattcacgttaatttaaaaatg |
| | ttctccataagcgtattattttataaccttactaaccacgattattaaaatatggctctatggtttaactccatatcctaacaattatta |
| | aaatatggctctatggtttaactccatatcctaacaattattaaaatatggctctatggtttaaccccatatcctaactaaggat |
| | gttaccattattaccatgtattaccatttcttctagcatcttctgtagagctgaagtgtatcttagagtcacctcttcatgaagg |
| | agatattgatgatatattttctcagctgctggtagatgaaaatatttctgtaatgccctcatgtaataatgataatttcagttgat |
| | acgcaaatatttttcttaaaaatatattttgttcttttatcctcagacatctaatatgcagaagataagatacagccagatttttct |
| | tccccgcccaccccccttggaaacaaaccattttcccctaccagaaacttgatggatgtttttctttatcctttgaccttctcta |
| | gctcaacaggaaatgttttcatttggggctcttttcttttttttttttttttttttggtgatggagtctctcgctctgtcgcccaggct |
| | ggagtgcagtggcgcagtctcggctcactacaacctccgcctcccgggttcaaacaattctcctgttttagcctcctgagt |
| | agctgggactacaggcacctgcccccacggccgactaattatgtatttttagtagagatgggatttcaccttgttggtcag |
| | gctgatcgcaaactcctgacctcaggtgatccacccacctcggcctcccaaagtgctgggattacaggggtgagccac |
| | tgcacctggcctattttctttaatgtaaatactattccataatcctgtttgatttacagactctttttgttaaaagatttcatttgttct |
| | tgagattagaattcgttgcaacaactgtggtagccttagtttttccttttcaatttactatctttaatgatagcatcctcagggct |
| | tttgcaccatgtatatctttacattcttaataattatttccatctaattaactttttactctgagatattgggaagttatcacagactt |
| | ccatttaacaaacttaatttctgcattttcaaatatgcttattactgtttatactgaacttatgaatttaactatttttacaggtatcc |
| | acgctatatccaactggactcccttgctaactgtcagctttcctttcataattaaagtgtcctctttaatcttaatcagagtatat |
| | cctaaaattaaaatgttctcttctgcttcttattttgaacaatggtatcttttcatatgtttggcgattttttttcctgcttactcatca |
| | ttacagaagatactctatgctgtataacatttgtattttagatgattctatcaaatattgcttatttcctgtaaacatctaaaagtc |
| | ttttaggctgaggagaagctataactttgttttaacagttactagttcaacttgtaacaaagttaaaacttttttttttaactttgct |
| | catttaaatatacagtggcctaaggaaactaagaaccaatcacagcagagtttgtgtaggtttgatggctaggaccaacc |
| | atgggaactgacgcatcatttcctgaatttagttgcgttcaagggtagtggtattcctgttctttgtctgacataactgtccac |
| | acctaaaagctgggcacagactttcccatctggtgggaaatatttcagaaacattagtgacacttgcagtgtcctgctattg |
| | ctgcaaaacttgctcttccttcagcccttgccatggaattcaggtataattgtggtcttctattggcacatcctttatttacaga |
| | catacctcagagatattacgggttcagttccacaccaccagaaaagcaattatcacaaagtgaatcacatgatttttttgac |
| | ttcctagtctataagagacttattttttcactttttaatcatagccattctgactggtgtgtgagatggtatctcattgtggtttttg |
| | tttctgtgttttgttgttgttgtttttgttgttgttgttcttgttgttgttgtttttgagacggagtcttgctctgtcacccaggctgga |
| | gtgcagcggcgcgatcttggctcactgcaagctccgccttccgggttcacgccattctcctgcctcagcctcccgagta |
| | gctgggattacaggcgcccctcaccaggcccagctaatttctatatttttatagagatgggttttcaccatgctggccagg |
| | gtggtctcgaactcctggcttcaagtgatccacccccctcggccttccaaagtgctgggataacaggcgtgagccacca |
| | cacccagcctcattgtggttttgatttgcattctctaatgatcagtgatgttgagttttttcttcatacgtttgtcccatgtatgtct |
| | tcttgacatgtatgtcacttgaaaagtgtctgctcacgtcctttgcccacattttaatggtggttttgttgttgttgttgttgttgtt |
| | gttttttgttttttgttttttgcctgtaaatttgtttaaattactgatagattctggatagtagaactttgtcagatgcatagtttgcaa |
| | atattttctcccattctgtaggttgtcagtttactctgttgaatttcttttgctatgcagaaattcttaaatttaattacatcctatttg |
| | tcaatttttagttttgttgcaatcgctttggcatttttgtcatgaaacttttgccagatcctgtgtctggaatggtatttatgagctt |
| | accttctagggtttttatagctttaggttttaagtttaagtatttaatccatcttgagtttattttttgtatggtataaggaagtggtc |
| | cagtttcaatcttctgcatatggctagccagttatcccagcattgtttattaaacaggaagtcctttgtccgttgcttgctgga |
| | gagattgcagagaaaagggattgcttatacactgttggtgggattgtaaattagttcagcaattatggaaaacagtgtggt |
| | gattcctcaaagaacttaaagaagaataaccattcaacccagcaaccccataattgggtatgtgcccaaagaaatataaa |
| | tggttctaccataaagacacatgcacacctatgttcactgcagcactgttcacaatagcaaagacatggcatcaacctaa |
| | atactcatcaacagtggactggataaagaagatgtgatacatatataccatggaatactatgcagccataaaagattataa |
| | gattatgtcctctgcagggacatggatggagctgggggctattatagtaggcaaactaactgaggaacataaaacccaa |
| | taccacatgttctaacttacaagtgggagctaaacagtgagaactcatggacacaaagaggggaacaataggcactgg |
| | gacctacttgagggaggtaggtgagaggaggaagaggatcacagaaaagtgcttattgggtactaagcttattgcctg |
| | gatggcaaaataatctatatccctgttacatgcagtttacctatataacaaaccttcacatgtacctctgaacctaaaataaa |
| | atttaaaaaaagacctatgtttacactatactatagtttattaagtatgtaatagaattacatctgaaaaagcaatgtacatacc |
| | ttaatttaaaaatattttcttgcttacaaatgcaaataatcatctgagtcttcagcaagtcataatctttatgtgggtggaggat |
| | cttgtctggatgtcaatggctgctgactgatcaaggtggtggttgctaaaggttgagtggctgtggcaacttcttaaaataa |
| | gactacaatgaagtttgttgcattgattgactcatattttcatgaaagatttccctttggcatgcaatcctgtttgatagcatttt |
| | acccacagtggaactcctttcaaattggagtcaatcttctcaaaacctgccactgctttatcaacaagtttatgtaatattcta |
| | aatcctttgttatcatttcacaatattcatagcatcttcactgggagtagattccatctcaagaaaccacttactttgatcattca |
| | tgagaagcaatccctcattcattcaagttttatcataagattacagcaattgagtcacattttcaggcttcacttctattctagtt |
| | ctcttgctatttctaccgcatctgcagttacttcctccaccgaattcttgaacccctcaaaatcaaccatgacgactggaatc |
| | aacttcttccaaattcctgttaatgttgatattttgacctcctcctatgaatcatttatgttcttaatgacattcagaatggtgaatt |
| | ctttccaaaagttttccaacttactttgcccagacccattcacggaatcattatgtatggcacctgtagccctaaaaatggat |
| | ttttttttttttttttttttttttgagacggagtcttgatctgtcactcaggctggagtgcagtggcatgatttcggctcagctcgct |
| | gcaggctccatcttctgggttcccgccattctcccacctcaacctcccgagtagctgggactacaggcgcccgccacca |
| | tgcccggctaattttttgtatttttagtagagacaaggtttcaccgtgttagccgggatggtctctatctcctgacctcatgat |
| | ccgcctgcctcggcctcccaaagtgctaggattataggcgtgagccactgcgcccggccaaaatggatttcttaataag |
| | acttgcagatcaaaatgactccttgatccatggagtgcgggcatgaaaacgttaatctccttgcagatctccatcagagct |
| | cttgagtgactaggtgcattgtcaatgagcagtaatattttgaaaagaatagtttttttctgagcagtaaatcttaactgtgag |
| | cttaaaatattcagtaaaccatgctgtaaacagatgtgctgtcattcagactttattgtttcattcacagagcataggctgaat |
| | acatttagcataattcttaagggccctaggatttttggaatggtaaatgagcactggcttcaactgaaagtcaccagctgca |
| | tacagttatccctcagtatgcatggaggattcgttccaggactcagatgaataccaaactctgtgcagtccctgatataaa |
| | atggtgtggtggccaggcatggtggctcatgcctgtaatctcagcactttgggaggctgaggcgggtgcatcgcctga |
| | ggttaggagttcgagaccagtctggccgacatgaagaaacccccctctctactaaaaatacaaaaaaatgagttgggg |
| | gtggtggcaggcacttgtaatcccagctactcgggagtctgaggcaggagaatcacttgaacccaggaggtggaggtt |
| | gcagtgagctgagattgtgccactgcactccagcctgggtgacaagagtgaaactccgtctcaaaaaaaaaaaaaaaa |
| | aaaggtatgtgtggtatttgaatataacctgggcacatcctcccatatactttcaaacatctctacattacttatgacacctaa |
| | tacaatgtaaatgctatgtaaacagttgtcatattgtaatgtttagggaatagtgacaagaaaaagttggctgtacatgttgg |
| | gtacagatgcaatttttaaaaaatatttttgatccatggttggttaaactcacagatgcagaatccacaaatatagagagcc |
| | aactatacttctaaattccataattctcaattttatcttttgtagaagaactggactagattatctctaaagtgtcttccacttctat |
| | atttcttccagtccacattaataggatcattgttaggctggtatctgtttctaaagtactacagagtaaatcaacaaggaaag |
| | caaggttgtcttctcattcagaaggccagaggttgctaaatactccagttctgaattgtaagtgaaacagggaagttatata |
| | ctaggtgattggtgtgctgttaaatgttttaaccttggctttgctctattaactttatctgtcctttttaaatcactttcagaaataa |
| | aataaggtgaatcacctcaagtggggtacaccaatgagttgagatcaatttgggacgtccttcatattgacctttgttgata |
| | caaattatctttgtcacagaatattgctgtgattccattacctcctgttgtacttgcctgagcttcccattgttttagtcactctgc |
| | atcactgtgtacgatgtcaattgtgttttagatggttaaaaaaaaggaaaagaaacatttcagtcataccaaagtgttgattt |
| | gcaataaagagtataatttactgtggctctgggtattgattaatctatacaatgaaaatggcttttgagtgctttggttatcagt |
| | tgaaattatctctattttttaagctgataattactgaaaaatgtcacaagtggaaaaaagggtgcaatacaaactagtgtttta |
| | tccagatatctattctgcctgggaaaggtaaaaggatacacattatggttgcaatacggtctggatgtttcacttctgtgtat |
| | gtgtatttcttgcccatctcaaataataggtgggactttttttcacttttattttaagtgaaggggcacatgtgcagaatgtgca |
| | ggtttgttacacaggtaaacatgtgtgatgagggtttgttttacccattatttcttttttaaaaaaatttcaataggtttttgggga |
| | actggtggtgtttggttacatggatgagttctttagtggtgatttctgagattctggtgcaccctgacccaagcagtgtacac |
| | tgtacccaatgtgtagtctcttatctctcaccccctcccaccatttcccctgagtcctcaaagtccattgtatcattcttatgcc |
| | tttgcatcctcatagcttagctcccatttgtgagtgagaacttacgatgtttggttttccattcctgagttacttcacttagaata |
| | atggtctccaattccatcaaaattgctgagaaagccattatttcattcctttttaagacaaagcagtattcatatatatttgtata |
| | tgtataattatatatgtatatacacatagaataatatatatattaatcaacaagtgattaatgggcgtttgggctggtgccatat |
| | ttttgcaattgtgaattgtgctgctataaacatgcgtgtgcaattatctttttcgtataatgacttcctttcctctgggtagatact |
| | cagtagtggaattgctggatcaaatggtaatatctacttttcgttcttaagaactctccacattgttttcagtaatggttgtacta |
| | gtttatattcccaccaacagtgtaaaagtgttccctttttaccacatccatgccaatatctattattttttgattgtatcaattgttg |
| | caggagtaaggtgatatctcattgtggttttgatttgcatttccctgataattagtgatgttgagcattttttcatatgtttgttgc |
| | ccatttatatatcttcttttgagaattgtctattcacgtccttagcccacttttgatgagattgtttgtttttttctggctgatttgtt |
| | tgagttcattgtagactctggatattagtcttttgtcagatgtatagattgtgaatattttatcccactctgtgggttgtctgtttactc |
| | tgccaagtatttattttgctggtcagaagctttttagtttaataagtctcatctatttattgttgtttttgttgcacttgcttttgggt |
| | tttgaagtctttgcccaagccaatgtctagaagggtttttctgatgttctagaagttttatggcttcaggtcttaatttaagttcttg |
| | atccctcttgaattgagttttgtataaggtgaaatatgaggatccagtttcattctcctacatgtggtttgccaattatcccagc |
| | accatttattgaatagggagtcttttccctactttatttttttgtttgctttgtcaaagctgtaaacattgggctttatttctgaatcc |
| | tgtattctgtcccattggtctatatgctgacttttatgccagtacctgctgttttggtgactatagccctatagtttgaattcagg |
| | taatgtgatgcctccaaatttgttctttctgtttagtcttgctctgactatgcgggctctttttattttcatatgaattttaagattatt |
| | ttttctagttctatgaagaataatggtggtatttttatgggaatttgttgtgtttccatttgtttgtgttgtctatgatttctttcagca |
| | gtgtttgtagttttccttgtagaagtctttaatctccttcgttaagtatattcctaagtattctattttttttttttgcagctattgta |
| | aaaggggttgagttcttgatttgattctcagcttggccactgttggtagtatagcagagctactgagttgggtacattaactttgt |
| | atcaaaactttgctgaattcgtttaccagttgcaggagctttttgaataagtgtttagtgtttcccaggtatacaattatgtcctc |
| | agcaaacagtgacagtttgacctcctctttaccaatttagatgtcctttattcctttctcttgtctgattcatctggctaaaacttc |
| | caatactatgttgaatagaagtggtgaaagtgggcaaaatcctgcactaaccaaatcccacagcatatcaaaaagataat |
| | ccactatgatcaagtgggttttgtaccagggatgcagggatggttaaacatccacaagtcaataaatgtgatacactgcat |
| | aaacagaattttaaaaaatcacatgatcatctaaatagatccagaagaaacatttgacaaaatccaacatcactttatgatta |
| | taaccctcaacaaaatcagcatagaagggacataccttaaggtaataaaaagccatctatgacagacccacagacgac |
| | atcatagtgaatggagaaaagttgaaagcattccccctgagaactgttgtacagatgatttcatcacctaggtattaaacct |
| | agtatccattcgttatttttcctgaccctctcctcctcccaccttgtcacacaccctctgataggccccagtgtgtgttggtttc |
| | ctttatatgttcatgtgttctcatcatttagctcctacttatgcctgcaaacatgtcatatttggttttctgttcctgtgttactttgct |
| | gaggataatggcctcgtgctccaaccatgtccttgaagaggacatgatctcattcttttttttgtggctgcataatattctgtg |
| | ttgtatatgtaccacattttatttatccactctatcactgatgggcatttaggttgattccatgtctttgccattgtgagtagtgca |
| | gtaatgaacatatgcatgcatgtgtctttataatagcgtgatttatattcctttgggtatatacctggtaaagggattgctggat |
| | caaatggcatttcagtctttagttgtttgaggaatcaccacaatgtcttccacaatgattgaactaagttacgctcccaccaa |
| | cagtgtataagtaagtgttcctttttctccacaatctctgcagtatctgttatttttttaccttttaataatagctattctgactggtg |
| | tgagatggtatctctttgtggttttgatttgcatttctctaatgataagtgatgtagagtttttttatatgctttgttggccacaaca |
| | tatatattgttggccacatgcatttcttcttttgagaagtgtctgttcatgtccattggccactttttaatggggttgtttgtttgttt |
| | ttttttcttgtaaatttgtttaagttccttatagatgctggatattagacctttgtcagatgcatagttggcaagtattttctcctatt |
| | ctctaggttatgtgtttactctgttgatctatatatatatatatatatatatatatatatatatatatatatattgctgtgcagaagct |
| | ctttagtttaattagatcataatcttttatcgtatcccttaactcaaaaaaatattgaacattaaaatattggtagcttttttcccac |
| | tatcaacctccatagtaaaactatagttaggctttccttcaaatgaaaatccattcattttttatactgtttgcctgaaacaaca |
| | cagtattctttcttaatagtattttcatgttgagctaaacaaacaacatttaaataaggaaaaaatattattatttcttttttattata |
| | ctttttttccttcttctttctttctttcttcttttttttctcttaatatatggggtctaactctatcacccaggctggagtctagggtgc |
| | aatcatagctcattgcagtcttgaaatcctgagctcaagtgatcctccctgctcagcctctgagtagctgggaccataggt |
| | atgtgccaccatgcctggctctttattacatttctgttataatattttctctattatatttcattttagttactaaagaatatgattttg |
| | aacttttttggaaagatcatcattttactctttgttgtagaagaaaatctatgatggtctaatgaactacggaaatattaacata |
| | attttaagaataatacatatatttgtattaccaaaggctatgcttattagtcattcaaacttcaactaattttttctttcatgaaata |
| | ctcagaagtatctattcaccaccaccaacactccctccatcatttaatttcaagcagacttgggtttattcctggaaatgattt |
| | tttactagtgttcttaattttagtcttcttctttctccaatatcttaaggcagaataatttttcttaaatgcaactttcattctttctc |
| | cataaatttgaggtatcttcacatgaaattgagcttcaaagaagatgaagatgagtaagggccgggaaagccagggaaggt |
| | ttctgtttcctgtgtatcaagtttcccaaacaagaatcttggagtcctcttcagcttctgcaactccttctcgatcaacatgca |
| | gtcaaacttcaaattgtattgacggaaactcagaactgcttagatcattttcttactctcattatttctgcgtggatttagacatt |
| | cattgtcttttgcctgggctcttgcaataggcttttatcttatatcctaccgtaaaaggtttcagatgctacacatcctccatact |
| | accaaagtaataggtaaaaaataaatctgattgtgtctgtacaaaaaaacctgttagaactaataaataaattcagtaaaatt |
| | gcaggatacaaagtcaacatacaaaaatcagtagtgtttctaaacactaagaatcaactatttgcaagagaagtcaagac |
| | aacaatatcattttaaagcatcaaaaatattgaggaataaacttaatgaggtgaaatatctgtacaccaaaaactataaaac |
| | atggattaaagaaattaaataagacacacataaaaagaaagatatcccgtgttaacgagtggaaagaattaatgttcttaa |
| | aatacccagagtacttaaagtgatctgtggattcaatgaaatccgtataaaaattccaatgacataatagatattttttaaatc |
| | ttaaaattcatatggaatcacaagttaccccaaacagtcaaagcaatcttaagtaaaaataatgaaacttggttgcatcaca |
| | ctgcctgatctcaaaatacattacaaagttgtagtgatcaaaacagcttggtactggcataaaaatcagcacacaaatgga |
| | acagaaccaaaagcccagaaataaatccatgcacttatggtcaataaatctttgacaaaagtaccaagaacacaaaatg |
| | ggaaacgaacagtctcttcagtaaatgatgttggaaaaacttggtatccacatgcagaagaatgaaattggacccttatct |
| | cacactatatacaaaaatcaactcaaaatggattaacaacttacagaacacctaaaactgtaaaagtactagaagaaaat |
| | ataggggaaaactctatgacattgatctgggcaatgattttttgaatctgaccccaaaatcacagaccaaaacaaaagaa |
| | ataatcaacagaatgaaaagacaacctacagaatggaagaaaatatttgcaaaccatatgttcaataaggggttaatgtct |
| | aaaatatgtaagaaactccaacaactcaatagcaaataataataataataataacctgattacaaaatgggcaaaggacc |
| | taaatggacatttctcaaaagaaggcatacaaatggccaacaagtgtatgataaaattcccaatatcactaatcatcagga |
| | aaacacaaattaaaactacagtgaaatagcacctcacattcattaaaaatggctactatcaaaagaacaaaagataagcg |
| | ttggtaaggatgtggagaaaaaaaagtgttgtacactgttggtgggattgtaaattagccatcatagaaaacaatatgta |
| | aattcctcaaaaaagtaaatacagaactattgtatgatccagtaattctacttctgggtatatacctagacattcaaagaaga |
| | attggtaccaaccctactgcaactatttcacaagatagagaaagaaggaatccaccctaaatcattctattaagtcagtatc |
| | acccaaataccaaaatcaggaaaggacacaagaaaaaagaaaactgcagactaatgttcctgatgaacatagatgcaa |
| | aaattctcaacaaaattctagctaagcaaatccaagggcatataaaaaagataatacatcatgatcaagtgggtttcatac |
| | ccgggatgcagggatggtttaacatatgtaactcaataaatgggatacatcacataaacaattaaaaataaaaatcatatg |
| | atcgtcttaatagacacagaaaaaatgtttgataaaatacagtatccctttatggtaaaaaatcctcaacaaaattggcata |
| | ggagaaacatacctcaaagtaataaaagccatctgtgacaaacccagaaccaatatcatactgaatggggaaaagttga |
| | aagcattccccctgagaactggaacaagacaaggtgacaaaattagtgtgataaagagatatttacactgtcgtgtttatt |
| | gcagcattatttccaatagacaggatacggaatcaacctaagtcttatctgtagatgaggaacggataaagaattgcaca |
| | cacacacacacgcacacacacacacacaatgaaatactcttcagccttaacaaagaaggaaattctgtcacttgtgaca |
| | acatgaatgaacctggaggacattaggttgagtaaaataagccaagcacagaatgacaaatattgcatgatgtaacttag |
| | gtatggaatctaaaaatgtcaaactcacagaaacagaaagtagaatggtggttaccaggggctactgggtagggctgta |
| | ggggtggaggaatggaaaatgttggtcaaagggtacaaaagttcagttaaacaggaggaataagttctggatatctatt |
| | gtactatgtaatgactgtagttaataatactgtgttgcatattcaaaaattgctatgtgtcgattttaaatgttctcaccaccaaa |
| | atataaatatctgaggtgaaggagatattaattagtttgatttaatcatttcacaatgcaaatattaagttgcacactgtaaata |
| | tatacaatttttaattgtcaattaaatataattgaaaaatagaacataataaaatattggattattaactctatcaggaaatgaa |
| | gctttgagtaacattttactgtttatatttatggttttagccaccaagcaaacatggtggtcaagaacttggactcacattcag |
| | gttgccatagtccaaattcaggcttccctactcactaatgtgttcttaagcaagaaataaacttattttggcttcctttggtaaa |
| | atgggattaataattgttcctatcctacattttattgtgaagattgaattaatgaaatatacacgtaaaacacttaagacaattc |
| | ctggccttagtaagatttcaacaaaaatggtcattattatgatgctttattctgttacatttatggattattagacagataaaatt |
| | ggacctcatatgtgaccattctattttcctttattcaaatggaaaaagaatgtcaattcaacatagggaaatactatatatgtat |
| | aaattattacttaaatttatgtttttacttaaatatatgtttattaaaatcaagtgtttaaatttatatattaacttttacatttataa |
| | catcttatttcttctgaaaacaaccaacacaacccatttcttatcagtgataattcaaatggcctctttattctttcctgaatttttca |
| | atcccaattcttctcttctgttctttttcagcagcatcttgcacagggctttatggaaaagactgaggtgctgtggtgaatttaatc |
| | tcctctgttgtttactttaatctcttcatccagttactctgacttttctccatgcttaatgttatccctcttgatttcacagcttaaca |
| | atttcttctttgttcttgaccccatcctagccttcttatttcaaaagtcccctcaagcaagtttcttttcacattcttaatttcttattc |
| | ttaataactccttttttatgccttcaagcaagtatggctctctctcatactgaaaatgtgctcttccgttctattttgctactgttttt |
| | attcacttaacagtaaatttcaaacaagttttgtgcacctaattctttcatttctttacaacccacccattgcttatccactcttac |
| | agctgtactagaattgtaatttaaatatgtatcatatcagtttttttgtaacattgataatttataatatttaacatattttgtaatag |
| | agttttcttcttaatattttactttccttttaaaaagcctcagcaagttgccattgactttcggcattgtatttgatacctctggact |
| | ctggctactatcgccctttctgtataaaatctgtaacttgaccaaggcaagaacctccaccccaggccaactaattttctca |
| | tctgctattgtcccaacctgctttggtttctcctataggtcagagtcaggtcctgcttgggtttgggcctttgctcataattttcc |
| | cttgcaattgaaatattctctcactccaggccagcctttaaagcctaacgtaagtaccaccacatccacgaagcctctgca |
| | catcaacttcagatctcaatgctgttaaccttttataaacaaaaagcaatatagaataaatcctttttgggagcaggagtcaa |
| | aatgctagttgcagtcttgcatctgtcacttatttgcttggccagtgacgtagccttattaaaaactcaatttctttatccaaaa |
| | gatgaggatgtttatacctccctgatctaccttctaagattgttgtacatgataagtaaaatactgtgcataaaaacatttgta |
| | aaaattttaaaaatctgttcaagcttgtcattgttaccacagataaatcgggtactaccttgctgtcttggattgcttttgtatgt |
| | tgttctcttatgttgttactggactcggtgttattacttaaatttctctgtatttattttgacaacttaattaggttttgaattctttg |
| | gaagcggagagtatgaatggtattatgagcaaagacccagaaggagccacctcctaattattttggagagaattatctaaga |
| | ggcctaattggacttgtgcttcccattaaagtatgattcatatcagagaagcttacagtcttattgctggccacatggagata |
| | gtacataggagatactcgatgggaattctgtcaactgaaagagagtcagcaagacagtgtaaacatggttagcgtgtctc |
| | agcttacccagcctggcttggtgtaaccaatatagactatggaaaataggaagtagacaaaaggtagtggagattgggc |
| | tcatgccattttctttaattgaaatacaatttccataaaataaaatacagatatttttaagtgcatattactttgagttttgacaaat |
| | gtattcccctgtctggcaactgctccaattaagatgtagaatatttctatcaccccagtacccttttcctgtcaatccctccttc |
| | tcccagagacaatcactgttccaatttctatcactatagcttagttttaccagttcaaaaacttcacataagtagaatcataaa |
| | gcatgttactttttttcctggcttttgtcacttaacagtttttttttttagatttaaccattttgttgcatatgtaagtatatcataa |
| | ctttttgttgttgagtagcactctcctatataagtatgttataataatgtttcatttacctattgatgagcatttgagttgttcctaagt |
| | tttaactagtataaaaaagtttactatgaacattcttgcataggtctttttgtgggtgtatattttcactactcttaagattgtcagact |
| | ggataaagagcaaagttcagtggtatgctgttttcaacacatattttaaatataacacagacaggttgaaagtaaaaaaatc |
| | agtaaaagatacagcatgaaaacactaagcaaaagaaagctgatatggctatatgaatatcaaaaaagtagattttaaga |
| | caaaggatactaacacagataaaaatggtcattttattggggaaaaaaaggccaatttgtaacaaggcatgacaatctta |
| | aaagggtataacaaagtagaatttcaaaatccaagaagaaaaaaatggcagaataaagtaaaaagcagagaaatccac |
| | aataattgttagagattttatcaattttatcccagtagttgctagaacaagaagacaaaaaatcaatgatatagacaacttga |
| | ataacattatcagggatcaaacctgcagacctaactgacatttatagaatattatacccaacaacttcagaatatacattcttt |
| | tcaaatacacatggaacattccacaggtggtgttatgctaggccataaaattagtgacacttcatttcaaagagtatttgact |
| | acaatggaattaaattagaaatcaattacaattagatatatagaaaaggcccctttttacaaaattaagcaacagacttctaa |
| | ataatcaatccatggatcaaagagggaaatcacacacacacacacacacacacacacacacacacacacacaaatac |
| | agtgcgctatacggagtgataattagaatacaacatgttaggaatctctgtgttcatgcctttaaccatttcatattatttcata |
| | ataatcaagaacttattaatcaaattagcatttaattatcttcagatgaaatgagaaagaggggggcaaaatgtagactga |
| | aggctgagaattataggaattatggtcacaatcagccagtgttttaactctactgcaattgtaacaaagtcattggatcttgg |
| | ttcaaccaatgggaatagatcctttcaaaatgccaataaataaaatgctaaataaacattttggcaattttctcttgataaatct |
| | cttatagcaatgtgcataatgcttccattttcatccaatgaagtactagtgaaaatagtctagatagtgttaatattgatagaa |
| | attataactaaggaataagttagatggtgattaaagagttgaataagcactcaaagaaagttgtgaaatctctgttctgaga |
| | tatgtgacaaaaccatctgtctctgattttagtttaatccttaatgaatataagaagcctgactaaatgatctcttgggaggcc |
| | cccttccaaacacaaaacccattattttaatttgcattggttatagtgccaacacaaagaaaaaacacactcaagatctttttt |
| | ataaagtagggatttaattctaaatcttagtcatatcttacaatttataaattctttaatgttgaaaaatattttctttgtagaacta |
| | aaaccattctctctctctccctccctccctctctctctttctctctgtctgtttgagaacccaaaataaaatgtagtttataatttg |
| | tgattatgatttgaacttttcaaaaaatgtatgagtcatttcttgccctttgataatttccaaatgatgatgctttgcttttgctttta |
| | gagatcagtctttgcttttaattatttccatatattttggttgggagtgagaaaaataaacggaaaatgtcactcatgagtgtc |
| | agcctggtaatgaatatatagaatgcaaatggtcactttactgggggagacggtaagtttttctgagccttttgcccttttatc |
| | ttctccagcctgctgttttttctgttttcatttgtagattaaactggtgcagcaatatggtggggtagaaatcacatgaatctga |
| | catatgtgggctcctgtatctgtgcacatggatagagatgctggtcaaaatccaaccatcttatgagttccagacatactttt |
| | aaaattcacatgcatgaagctatacaaaatattttaaaatacagaatccaaaataaaatttctttttgtataaaatactccttct |
| | caattcattcctgtaggcaaaacttgatatttaataatattaagaaaaagaagaaaatgaattcgaaatattttattgctttaca |
| | acacaaaaaaaatggtttacttactcccaataatctgaatttattccaatagtatatttatggatttaaccaggagagggatta |
| | tccttattctaagtaggggagaaaaaattaaaaaaaaaaaaaaagaaaggaaggaaaaactccagtggtcaagagatt |
| | gagtgaaccgaggaacagagtggttatttgttctctgtaatgtattaggaatgactggaagtaatggaatatgctagtgaat |
| | atagctacccctatattgaatagaaaaatacattaccaaaacacttttcacacacgaaaacaaaagaacattttgtacaaat |
| | acctgatgtgtagctatatcatttagttgctactattgcctagtttaaaaattgtaccctggttaaaaacagaatcccttttctaa |
| | tcttttaagatttcaacaaatatttccaggcattaaggacaggcagtattttgccttgtccacttctctgacgtctagctgtttct |
| | agttgctaaatttggctgctgtacttttcatatccgtaaagctgttcttgggtccaccctgcaggcagccatccaggtttgga |
| | tttttccgggcagcccgggcttggctatcctgctttcatcatcctccttctgttctcaagccaaggacatagaatggcagag |
| | agctcccctttatttctggtatgattgttgcttctccgggtctggcttgggctctgatcttgacatttatgtcacaggtgtcact |
| | ggtgaaactgctgtgaaaccagatggcaggcctaactgaggttggcagcaactgctgttgtccccagtgtcactgctac |
| | ctgctctactttcttattcctgttgatgttctactgccttgttttattgcattcgttcaatgttaccttccaacaggccttttcaaatg |
| | tgaacaccattccataactcttctgatgcattaattccccatggaacccattcatctagggaggctcatgacttggctagatt |
| | ctccactttccttcagggcttttgttaaacctcatttcttcagcaagcctctccatgaccaccctatctaaaattgcaatacatc |
| | cttctcctccccaaccctgattattttttctctctttttcataaattttctttccctggagcaatttgtaccacatgtcagaatctata |
| | tttgccttacttgggcattatctgtatccctccactgggttacctagaatataagctccacaagggcaaggttttgtctgtttg |
| | gttcattactgtatcccaagcatctggaagaagactggcccttagtagacatttcaaaatttttgttaaatgattagctggatt |
| | gtaaatactgaaagattaggtttattgtttttgatttggtttcattcattgtgcttagtacagtgctatatgtaagagcttgaaag |
| | acagagaaaagagcattagtagtatctgttatgtatactggatgttttacttgagttatacccttttttttttccaagacagagtc |
| | ttgctctgttgcccaggctggagtgcagtggtgtgatctcggctcactgcaacctccgcctcctgggttgaagcaattctc |
| | ctgcgtcagcctcccaagtagctgggattacaggcacccgccaccacgcctggctaatttttatatttttagtagagacag |
| | gttttcaccatgttggccaggctggtcttgaactcctaacctcataatctgccctcctcggcctcccaaagtgctgggatg |
| | gcaggcatgagccactgcactcagccaagttatactctttaatcctcacaataatctaggtattttgaccaaagaaccaca |
| | gctagaaagtaggagagtaagatgtaaaccagttttgtctggctctaaaattactgcctttttttgtttgtttgttttgctcagaa |
| | gaattggtcatgccaaatagtaataagagttcttcaaaatgaatgatatttcataataaggtgtagctagaaacagtttgcca |
| | aaaatacttaggaaaaaaaaaagatttgtgaattagctccctaaaaatacaaacgcaacaattccaaaagaaggaattcc |
| | aaagttattgggccatagtacttttgtgttaagtgcctggccctccaaggtaactaattttaatttaaaaaaaatgtctggaca |
| | catattttgacatgttttgctgaaaatatatttataatatgagcccaacaaagacaaatagattcattcttggtgctaaaaaatg |
| | acaagaaaacttctacgccattgctctcgaatgctttcttcattgtagtccaagaaaatttgattagttgtattttaaaagtaag |
| | ccagatttcactggtaaggtctttccttacttcatcttctcccttgacaccttttgatttggttgtacaaatgcaggttaggccc |
| | cattattaattcagtaccacatatttatgaagaccccacatgatgcaatcagcactagagactggaagtttttcatcaagcc |
| | cagtaagcccacagtaaaaaggaatcagtctcaatcactcatcaggaagctaacatgttctgtgaccaccttctggatgtt |
| | gagttatctctaatccagagtttatgtcaatgatcttaaaaattagttcaattctggatgggcgcggtggctcatgcctataat |
| | cccagtactttggaaggctgaggtgggcggatcacgaggtcaggagatggagaccatcctggctaacatggtgaaac |
| | cccatctctactaaaaatacaaaaaattagacaggtgtcgacagagcaagactccgtctcaaaaaaaaaattagtttaatt |
| | ttgtcttcattttttcttacttagtaaaagataatgtaatgcctggtgtttttttaaggcaatcatagaatctggaggttgaaaata |
| | tttggggaattatacattctaattattcagacaggcatccttactgcagtatctgctagtaattgaggattatttcatatttaaaa |
| | atactcatatttttatgcaaatctcagaatttgaacattgttacttataattgttaaaaagtaaattgagaaacaataaaatgtta |
| | aagagcttattgagcaaatggtaattcttgaatcagctccaaaccaaaagtggttctgaggccacttgagagaactcaaa |
| | gagaaagcttttatagggagaatgtagaaataaagcaaaggaaatatttaattggttgcaattatatagttgccttatttggt |
| | ctatcttgctgaaaagttcctacttgtattactacatgagttggcagcttctgattggttagccttaagtttcatttctctttaggc |
| | atttataagaaatagttcaagttgagttttgcatatgtttgcaaatcaagcaaagtttaggttacttctgagggccacttggatt |
| | tgtctgctcagaacttcttcaagcctggtctgttttaatttacttcaacatattttatcaacattttgctttctataacattcatagta |
| | accttagatctgccctctagaatcgacagaggctatttcacccccagcctcttttcaacatgagagtttgctcaatgctgctt |
| | ctatttgaaatcttctgttattcagctcttttaatgaatatctactttctgcaaggcattgctttatgtactgcaagagttacaaag |
| | aggagtacatcataatcttacttgaaggcaattacttaaatagaaaaaatggtgtgtgaaaaacaaatgttcttaagcttga |
| | aaaagtgaaagtgtgtacccagctggaaggatgtgaaagtgtgcatggaaaatgtatttgagatttaaactgctgttgtctt |
| | ctgggttgaattggtgtcatgaacttcaggtagtgaaaatagcttaaacaatacccctgaagtggaaacacagagattagt |
| | gtgaaaatttagaagaccgtgagcacattatattttgtgtgtaccataagacactggtagagtatattagtccattttcttgct |
| | gccataaagaactgcctgagactggaaaatttacaaaggaaagaggtttaattgattctaagtttggcatgactggggag |
| | gcgtcaggaaacttactatcatggcaaaaggcaaagaggaggcaaggcacctttttctccaggcggcaggaagaagt |
| | gctgagcaaagaggggagagccccttataaaatcatcagattttgtaagaactcactcattatcacaagaacagcactg |
| | gggaaaccacccccatggttcaattacctccacctagtctctcccttgacactcggggattatggagattaccatttaagat |
| | gagatttgggtggggacacaaagcctaatcctatcgtgggggttggtaagagggaagactagaaaggaaagctggac |
| | taggcaaagtggctcatggccataatcccagcagtttgtgaggccaaggcagggggattacttgagccaggagacca |
| | gcgctggcaacatagtaaaaccccatctctacaaaatgttagaaaattagacaagtatggtggtgtgcactatagtccca |
| | gctacttaggaggctgagatgggaggatctcttgggcctgggaggtcaaggttgcagtgagtctttatcacaacactgc |
| | actccagcataggcaacagagcagaaccttgtgaaaaagagagaacgagaaaagaaaaagaaagaagtcaagaaa |
| | gaaaggcaggcaggcaggaaggaaggaaggaatgaaggaaggaagcagggaaggaagcattctttacatgccag |
| | gaagaggaatcttagtgatgagaaaactgagtctctgagagtttgagcctcactcaaggtcactgagttgctaaatggca |
| | gaactggatttcaatcccaataatttgattcaaaacccatactctttcccatataccacattatctaggaggtgtcccaagag |
| | ttctgaagggggagttagcttccatctcaggagacgaagagagtcttcttagaccaaatggtatttgagataattttgataa |
| | gtttatgttgaattcattggttgagatagggaagacaattcatttcaaatggaagaaactgagcaaaggtacaaagggga |
| | ggaagtggagagattgggaacagaaaataattaatctttggttgaacatcatcaaaaaggtataaaggatggaaatgaa |
| | agctaagatcagagcacaaaaggataacatatttgaacttttacttggacttaaatgataaagtttttagacagattattatta |
| | taactacagccaagtttttaaaaatattatattagtaattatatataaaatagatggatgggaaacaaacggaagaatggag |
| | attatctgtttagagtaacccagttaattataataaaatattgaatctgcatgttggcaatggaatgagaagggaagagata |
| | gatatgtaatttcagaagcacaattcacagaacttgatagagattagtggaaggaggagggagatagatgacattaattc |
| | tgtgatatggtttgcctgtgtcccacccaaatctcattttgaattcccacgtgttgtgagagggacccagtgggaggtaact |
| | gaatcatgtgggcaggtgtctcccgtgctgttcttgtgatagtgaataagtctcataagatccgatggttgtataaggggg |
| | agtttccgtgcacgagccctttttctttgcctgccaccattcacgaaatatgtgacttgctccttcttgccttctgccatgattg |
| | tgaggcctccccaaccaacaggtggaactgtaagtccactaaacctctttttttttaaattgcccagtctcaggtatgtcttt |
| | atcagcagcgtgaaaacagactaatacattctgtaaatttgaatgcaggagatagaacctttaaaagcaatggtaatttgt |
| | cttgcaaggcctatgtttatctctactatgtactcagactattgtacagcctggaccatagaaggtatgcaatacgattattta |
| | tctatgtactcaatccctactctgtgccagggattgtcctaagcactagcgatagagtaaaaagaaaagaaaaaactcttg |
| | ttctcattagtaaaataaacatcacgttgcttcacccaaactagatcaccttagtcccttttattgttatctctgtgtcactctcct |
| | ggcaggtgagtgtttctgcttttaagaatctgtacctgtgtcttgctttagataactgtcttcagattatctgagtcattttttcca |
| | cttcagcaccaagtctgaagtgccggtgagtttacgtgcccccacccatatccttagcgaagactgactattgcagtcttg |
| | tgaaagtacagatcccttgcttcatatagggacaaactcaaatacataatttgtactccaaagttgtcctgtgggatcataat |
| | aaagccacttctgtagggctttgtttggaattacaccttagcttggctccttccccatccagacctgcagctttacccactcc |
| | tttcctgattgatcctaggagtacctccttaactaatcactttcccattaagccacatctcagtgtctgtctctggggaacctg |
| | acctaagacatgtagtttacattgcaatgtacagagacaaataataaacaatataaataattattaataagtttcatactagg |
| | ccgggcatgttggctcatgcctttaatcccagccagttgggaggccaaggcaggagaattgcttaaagccaggagttca |
| | agaccagcctggccaacatagcaagaccctcatctctacaaataaaataaattagccaggcaaggtggcatgtgcctct |
| | agttctggctacttggaatgctgaggtgggaggatcactggagcccaggaggtcgaggctgcagtgagctttgccact |
| | acacactagtctgggcaacagagcaagaccctatctctaaaaaaatagatattttattaggggaagttgaagttatagag |
| | ggaaataaggaaggagatgtggttagagtgtgtgaaagtgtgtgtgtgtgttttcagttgtaggggatggtgttcaattttat |
| | atagcatggttggggaaggcatcactgggaaggtcctatttgagcaaagactagtaggtgctaagggaatgagccatg |
| | caaatttctgggaggaaagcatttcaagcagagatgaaagcaagtgcaaagccatgagctgggagtgtctggagtgct |
| | tggtgagaagtagccatgtgagaaagtacagggcaggagacgacatgagagaaggaagtgaggggggtgggcag |
| | accttgcaggccaacacaagccatggctcatactttgacttttcctctgagtaaatagagagtcactgtagagttctgagc |
| | agaatagtgactcgactgtttttgcgtttcaaaggaatctggctaccacattgagaataaactgaaggaggccaaaaatta |
| | agcatggagaccaatttagacattattaccagggctggcttcgtgggcagccacacaaggctccatgctaagaagggc |
| | cccacacatggagcctaatgctctgcagtcaccatcttgataattttaataatttgtgtttgaatttaggttttgtaagtgaaag |
| | tcatgggactgtggagcatgttctgggtgctcagtgtctggtacttcctgctgtggataggttcaacactacctgcacccct |
| | gctccctgcccaggccactcagtgccccctttttcacctttttccagcaactattgccaccctccaacagggagaatgctg |
| | ggctgctggtgggaagatgtctgtgttctgctgcccttaagcctgtcggggatttgatcacaagctcaggtcaagttgca |
| | gttaggaatgcacctcaggctggaagtgccaaggcaggtgagagactcagtgggagagagcttctcacccacctcta |
| | atccaggcatgtcaggtgcagagttccaatgctttggggggttgaccttccaccagtttgggtggcaggctcatgggaa |
| | gggtagattgacttctctgaccccaattgagggcaggttgcttcagtccagcaactggtgggaaggggaatttggcagt |
| | gggggatgggggcagacattatcttctgctctgaatcagggacactcatatggctctgtgtttgccctgcaagtgtttccct |
| | gctccaatgagtacaacattaaatagcaaataaaagccatcagatcagtagacagtttgcataaataagaaaaaagctat |
| | gttgcataatcttttaatggcccctttttcctgctacttaaacaagtgtcttcctcttttttttagagacaggtcttgctatgttgct |
| | caggccagtctccaactcctagcctcaaggaatcttccagtttgagcctctggagtagctaggattataggtgcaaggca |
| | ctactcgcagctggggcctcaacattatcagttttgcactgtacttcataaattactggccctagcaattgcagtaatctaag |
| | tgaatgatggtggcaacttgaaccagtgtgctaatggtagaggaggtgagaaatgattggatccaagatatattttgaata |
| | tagaacaaaaaggtcactagaggttatgacatagtgtatgagagagagaggaaggaatcaaggatggctgtaaggtttt |
| | tcacctgaggaacttgacattatttgttgaatacataaatgatgtgtcctgttatgtatctgttcatgaaacacattgatggtaa |
| | tgaagatgcagtggcagaagcaaatcatagaagtatttcctagcggtagaaatattaagacttggtgacaaagatcataa |
| | attaaaggaaagggagtgaaacaactaagtcaggaaaaatacttctaacttaggaaaaaagaagaaaaaggaaggaa |
| | atggaaatatttaacaaatggtataaatttctaaggaagataagttcaattttggaaatgttcacctgaggctccaagaacct |
| | acagcccaggagaccttagctataggttgttgggaatatgtgataggatcctgaatgagcactcaggactagggatctat |
| | gttgagggcatcatataagtgtaagctggagtcacaagaatggttaactcatcaataaatgaatatgaggagagatgtat |
| | acagtattgcagaagagagttccttgtaaatgtgtaagctcatcctccatgcctgggataagagtacattcattatggtgca |
| | tgtgaaggtattgagaaaacaacttccgctttctggcagaggttaagctagtcccataatagcaatagcaagattataagg |
| | ggaaactatgttatttgtactgggtcacaaatttgtctaatagcagtggacctgtgggattctataaacatcttaggtataga |
| | aagggatccagtgacttttagaaaatcagacttatatcatatttcccttcttaacttgtagctcagtggtgcatttcttaccctg |
| | ggggagaaatggaacatggactatatttagtatttgaaatcagacacatgtgaatttaaactctggctcagagtggcaatg |
| | gcaaattattcaacatccatacaccctgatcactcccacatttgaaaagtgacaacaatactacttccttcacaaataatgc |
| | aaggatcaatgagataacaaatcaagaagccagtagtgtgccacaatttaggtagtgttgacattacatgaagaaatgaa |
| | cccatggtctcctatgctctccttcactcctctctagttctctctctctctctttctctctctctctctgtctgtttttctctctctct |
| | catacacacaggcatgcatacatacactttggaatgtagctattttttgagcattgttttattattatttaatttatttatttatttat |
| | tttttgagacagagtctggctctgtcgcccaggttggagtgcagtggcgcgatctcggctcactgcaagctccgcctcctggg |
| | ttcatgccattctcctgcctcagcctccctagtagctgggactacaggtgcccgccaccacacctggctaattgtttgtattt |
| | ttagtagggatggggtttcaccatgttagcaggatggtctcaatctcctgacctcgtgatctgcccacctcggcctcccaa |
| | agtgctgggtttacaggcgtgagccactgcgcccagcctgttattattattatttcaaagaccttctgatcctggctctacct |
| | gctcattcatggggaagatgcccagacattcagatctactgttccactgaggagtttagaggctgctttttctcccccataa |
| | aagagaatggggactgatggaaaaattttaggtagaagtctttgatgaaaagaagaaatgaggaaaactgagctggaa |
| | atattcaggtatatcaaacaagtggaaatacctcaccctcataaagagaaccatatcctgggaaattttctttcttacctgatt |
| | attaaaatccagaattggaattctgtgattccagtataatccatccatcaaaaccaaactactatgtaatcagattagaggtg |
| | gccttcctgctgatgagccatttagagacggttgaatgagttcttggaagtctgctaacatgcacttaatacatctgaagatt |
| | ttaaaacgttgcagcaatgtattgccagtctacctacagtaaaataaaaatgcttccattaaaatacaggtatatttcttttata |
| | tagttaacttatttttcctaggaaagttagatgtaaagtgattatctatacactgaatcatatttcccagagatcatcatagtaa |
| | gattggtatgcatttctgttaaagaaaagcaaaatgtgtcttaggacataacaatcatttaatggaatatctaaagaatgttg |
| | gtcaagcattttgaacaagatgaagatggaaccagggctgggtgtgggcagtagactgttccaccaggagccaaagat |
| | gacagtgaatatacacttgacatcagtcaccaatgacaacctaattctacgtggcatactggcctggatcaatgagtctcc |
| | tgaagttgaatttgacaaagatggaacaattgtgctcaggggctgcttattgttgctttatggtcatcctcttccctggctcca |
| | ttgccttgaagaaggtgaaattccatgaatacattcataacttcaaaatactacaagcagtatttttaagaggggggtgat |
| | gacaaagtaatttctgtggacaattaggaaaaggaaactttcaggacaattttgaattcattcagtactttaagaagttttttg |
| | atgcaaactgcaatgaaaaagactataaccctgtggctgccggacaaggtcaagaaactgaagtggctccctctattgtt |
| | gctccagttctgaataaaccgaagtaacctctcagctctagcaatccagctccacataggaccatttccacaaataactgc |
| | agcttctaaggctgcagcttctaaggccccagggtggtattaaaaaatcctggtgtgggcagtaggagtgctgcagggt |
| | caaattgatgctagcaggtcagtgtattgaaacttggcattgaagccttggagaaagagatttctacttcagaaagctaag |
| | gaacaatgaattgatttgccagcagaacgaggggcaaaatgaccctgtattgcagaggactgtagacatgctctgtgcc |
| | acagatgaagacttcgtgatatccgatgaaaggggcccacaggaggaacaagaagagttttaacaacccgaaccagg |
| | agagccttatccgaattctgcactccaaatcttgtgcttaactgttaaatactcccttttattattcttagaagactcactggttt |
| | ctttttatgagcatgaagtacctctttttaaagtgcattttgcagagtttcactccttttttggtgagtttgagttgggagttttact |
| | gtgcagcagagcaacattaacatctggttagttcacctgggaaacaaagaggctgaacatggggctcaccatgtgcat |
| | gtaggtaacattgattgctggagaaggtgtttggtaatatgccgaagtggagaccttggtacagaaatgtgaagactgaa |
| | ttgaattttaacctaatgtgaaatcttggtagagaattttgtaataagttaatgcctaaagaatatttaaaatatgcttccatatt |
| | caaaatatcaaatgtaacacatcagaacatcttatgtgtttgacattgtatgttggaaggaagggccagacatcagaacct |
| | ttggaacctggtgtcatcacaggccttacagggctgcttgcaacctcacaggcctagactttggccccaaaggaaagtt |
| | ctaaatgttgctctgtaaatccatttggtgtcattgaccaactgcatccaggctaaaaagcaagaggtattgttgcctggat |
| | gaacacagggtgtgtttcagccctgagatttttgggttgaagagctctattttcattgaggatttctctggaaatttttccaatt |
| | atctctgaaatttctatgtattacgcttttttgggaaatgaggtgtgtccaattctttcatctaacaacgcttttggggatttgctc |
| | acatctctgagatttgaatggaggttgtttcccattttaccatcctttagttttacatttaacatgtcaccagtggtgaatccata |
| | gaggtctgcagcaaacttgatccttgcctcgttggaggaaagaatttggctgaggggaagaagtaggtttaaggtagag |
| | ggagagaccgaggcaagttttagagtaggagtaagagtttattaaaaagttttagagcaggaacaaaaggaagtaaagt |
| | atacttgggaaaaggccaagcaggcaacttgagaggtccaagtgccctgtttggcccttgacttgaagttttatacattgg |
| | cctagttatggcctggttacggcgtttgcatctctcttccctttattttcttttggagcaggctgtacacatgtgcagtgatctg |
| | ccagcacttgggaggggcaatatgtgcagtgtgtttactgaagttgtgtgcatgctcacttgaggcaattttcctttaccag |
| | tagagtgttcctgagaaggtcatatgccagttaaactctgccattttgcctcttaatgtgcatgcttgagctcactcacccaa |
| | ttcctgagaacttatcaggaagcctctgatcaccaggttcaggtgttttttttttttaagccattaggggactgtctttacctgg |
| | tgctggctaagaccaattattattttagacagtttagtaaccacctgaccgtcatctgatggttgcctgacattcctgaggga |
| | ggggctctctactgccttgctcatgtctgcctaactacctactctaacattgcttctcttctctgctccccttgcccactgggg |
| | acacctctttgggttcttaaagtttgcagcttggagttggaagtgcagcaggcaggtgggcacactgcaaattctttgtgg |
| | acctctggcaaagggaataatcagggaaggctctggttacctctgcaaagctgggatgtttttggtatctgctgtccacag |
| | ctctccagttctctgaatactttatcagtacactaatctcttaagagataaaatttattagtgtgttgctaaatgttcattttctttta |
| | cagaaaatacagtaccatgtctgaattattcatatttaaaatttgttattccttaactctccctcatttgatttgtgcacaacctat |
| | tccatccttttgtttggcagaagtttgcaaaatatgtgtcactcactgagattgttcagcccctgatgcatttgtattgatttgtg |
| | tctggtgatagcttgtcctaaaatgtgtgtaaaaagtaaacattttataacaaaattgttgtttaatggatgctttgtgtggaatt |
| | cagagaaaaatccagattcagtgattaacaatgccaaaaaatgcaagtaaatagccattgttcaaatgacagtggtgctg |
| | tttctcttttgtggttttttagacttttgttacctaaaattctgctttattagcaactcatttttcacctgatgtttcttgacaggctt |
| | tttttttctattttaagtagtttctaaataatttttttttatttcaagaaagagaaaaaagaatattgctcaatgcacctaatataaaa |
| | ataaactataaaaaataagttagtttcatattggaactaaacctagcattaaaaaaaaaaaacagcttttctcaccagtaaaca |
| | ctgaacgtgagttcttcatattctactattctctgtttctactctacagcagaaaaacacgtaagtctggttgttcagaactgtc |
| | tccccattatggaaattgttagttacagaaatgggctagtggattcaggttaaaaagagaaggatacatatacatgtgtgtg |
| | tgtgtgtgtgtgtgtacatatgtgtgtgtatatgcatatatacaaatatatatcacagcaagtaacaatcaaaaagttgataag |
| | aaaatagacttgaacctatggaataagaaagctagaatatctgaaaaataagtattcaaataatatttagcaaatcttttattg |
| | gtttatgtttacattactgtaatgtaccaaagaacccaaaaaactacaattgtagaacagaacttaaaaggccacttccaga |
| | aaaaaagactttccttagtgttttcagctttcttaattcttgggcacaaaattttctaagtctttgcgcacttatccacaaccact |
| | aattacaatataaactaatataaaattatgtcctcatctatgaaatttaatatgccattttcttagtataaatttatttctagagcat |
| | gtattattaatgtggatctttgtagaaaaaaattgtggtacaatgtttattactacatacccttatttttttaaattgtcttatctttca |
| | tataactttttgctgatgtcatcatgtgtaataacatgattttttaaaattttaatttagaaaaaatatattcctcttgtataaatgag |
| | aaaactaaaggaaaagagtatcgaaaggaaaacattatagaaatatcagaatttagaatcccaattcttaagattttttatttt |
| | ctgactacttcaaataatatcgtttgcctttctcattattcatgggtcttccaaatatagtgtctggccttaaaagttgtaacaca |
| | aaatcttctgtctgaaatgtagtacaactgaatgaaatttaaaaccttcacgtaatatatgtcatatagaactctcataaaatat |
| | cagtatgatgtgataacaaaggtcagaacaaatggaagaaaaaccaatgtatgatttctgaaaaccttatcctgggaaat |
| | gagaaacagactataattctttacttacatctacattatgcaaaagtatctttgatgtgaaaaaatactcagtgttgttcattcg |
| | atgttttatatgttttggtaaaacattttgtgttcacccttttaaatattccaagacacactcactggctagaatcaatgggaga |
| | gaaagaggtgactctataactaatataatgttaaggaaactgcacatatcttggtatgtttctgatagtgcataattccagaa |
| | aatgtcttaagcattgcataattgttagaaacatgaaatctgcagccagcaagcatgagttccacatctgaccctactctttc |
| | cagttgtgagaacttatttgtattttaaactactgaatttcaggatgcctctgtttattcctgtttctttattttttaagttgacagtat |
| | ttgtacatattcatggggtacatagtgatatttcaatacatataacatatactgatcagatcagggttattagcacacccatca |
| | tctcaaacatttagcatttctttgtattgggaacattcaatatcctcctcctacctatttgaagttctataatattgttaagtatagt |
| | catcctacagtgctatagaagctataacttatttctcctgtctagctgtaattttgtatcctttaacaaatctctccctatcttccc |
| | cagcctctagtatcctttgttccattttttacttcttcctcatgttttaaagggaaatgattacagtatctatttcattaggttgctat |
| | aaggaataaatggtttcatagctcttaaaatagtgcttgacaaaagtaggtactatgtgtagatttttattaaaaaaagtagat |
| | actatgtgttattattatgactaatcttctcccaaaaatatgtttggtaaatcatttcataaatttgtttattcttaatttattttgaa |
| | ttagaatcatttgtataacaagttggtatattgctgagcacatctgtaaagtaagacctgaatatggatttgataattgaagttg |
| | acactcatcatagagatttatgaaatattgtgcgcttgattgcagtaggacaacttactatggggaaggaatcttgtaagtat |
| | cttgcctggccagtatacatggaagttggtacacatggaagatataccatatgtatatatcccgatattgataatgttactttc |
| | caaaagtggaggcactgcaaatatgatgagaatgtgactagagcacgcacttgtttgttctttctgtccagcattaatttcc |
| | cccttattccagtaatagtaactcaacaactctgctctttgtcatttcctggtgtttttttcttttcttttctttcttttttttttt |
| | tttttttttttttgagactgagtttcactctgttgcccaggctggagtgcagtggcatgatcttggcttactgcaaactccgtctcctg |
| | ggttcaagcgattctcctgcctcagcctccccagtagctgggattgcaggtgcccatcaccatgcccggctaattttttctattt |
| | tttagtagagatggggtttcatcatgttggccaggctggtcttgatctcctggcctcaagcaatctgcctgcttcggcctcc |
| | cagagtgctgggattacaggcatgagccaccgggacttgtctgtcgttcgttccttccttccttccttccttccttccttcctt |
| | ccttccttccttccttccttccttccttcctccctgtctgtctttctttctctttctttcttttttctttctttctttcttttttcttt |
| | ctttctttctttcttttctttctttctttctttcttcctctcctttctttccttccttccttccttccttccttccttccttccttcctt |
| | ccttcctttctctctctctctctctctctctctttctttctttgtcttgctctatctcccaggctggagtgcagtggtgtgatcatagct |
| | cactgcagcctcaaatgcctggtctcaagggatcctcctgccctgatctcccaaaatgctgggattacaagcaaaagcaaccttcccagc |
| | tccaactccacttttcagaaatgcttctgtaccacaatgataaccctgactactcataggtttcaaatgaggactcttccgtg |
| | tttcaaggctacagcatgtggcttaggagaggccaatcaaagacggcattaccctggacagagtaattacttcaggatg |
| | ggcatgtgacccaccaggtgtcagtgagatgcaatgagtttttcggagtctttcttcccactgaattgaggtctggaaggtt |
| | gggaggctagagctgctacagcatctagctgtagggaagctacggctaagagaagaatctttaggaaacttatcactat |
| | gaaagtcagaacaaaaggatacagcataactatgagctggtaacatcagctgagctcttgaatcatcatatgcctaagac |
| | tagccctggacaaaccatgaaccaataaactcctgtctttatttattttttacttcaattagtttgagacatctttttctcttacaat |
| | caaaagttatctgctaaagtgactaaaatattattttgggctacatcttccccttgctcatttccaacaaacacattttcttctttt |
| | ctccaaatcctttattttagattcagtgggtacctgtgcaggtttgttatgatggtatattgcatgatgccaaagtttgagggc |
| | acgattgaacccaccacccaggtagcaagcatagtacccaataggtagtttttcaacacttgcttcccccaccctataccc |
| | acttatagtccacggtgtctcttgttcccatgtttatgtccatgtgtacccaatgtttagctcccacttataagtgagaagatgt |
| | ggtattttgttttctgtttttgcccaacaaatacattttccaatgaaaatgatgaagccagaaagtatgaaatacattgatttaa |
| | caattagaactgttctaattataattgataccggtgcctgtttttttttatgattgagtaatgatcacagatttttgctatcagatt |
| | ttgagtttgaatcctgcctgtatgatcttaggagacttttcctaatctcagtgtatgtccttacttcagtttgcaaataaggataa |
| | tgaaagcacttatccaatagggttgtgtggaggatgaaataatacagggcaaatgtcttagccaagtgactggcacttag |
| | taagtcctcaaatcttcactatgatattaattgaaagcctagcattcacaatctcactggtttatcagttcttttttctggttgcttt |
| | ttagtgacttaaatcagagataaataagattggtgtctcactgggttaatattttttctggagagagttaacttgtgtggcaca |
| | ggccatctctttattgaattagcaaagaactacattggatttaagattatctggggctgaggctaatatcaaatcctactctat |
| | gtttgtagttccttggctacaagcctacatacggtaattgctgtttaatatgaatttttatttttaacccttacatatagtaagggt |
| | taattgctatttaatatgagtttttatttttaacccttccctatttacgtcatgctttgcttaaggaccaagatatattctgagaaat |
| | gtgtcattaggtgatttttgtcattgtgtgtacctcatagagtgtacgtaaaacctagatggtagagcctattacacaactag |
| | gttacatggtatagcctattgttcctaggctacaaacctatactggatgttactgtactgaatactacaggcaattgtaatgc |
| | aatggtatttgtgtatctaaacacgtctaaacatagacaaggtacagtaaaaaatatggtattataatcttatgcgaccactg |
| | tcatatatgtgatctgtcattgaccaaaaatgccattaagcagtgcactactgtactttctctttactttaaatttgaattctttat |
| | atgggttgaatgactttctgacatagcaaataaaaagcatgaggagaagcattatctgttaacaaaattaacacttaaaatc |
| | aacaaagttttaatgtttcgttccaagaaaagcctgtggaagatcagttccacaactgagagctttgggctgcttcagacat |
| | atgtctgtgtgtacgctgtgaaggtgtttctcttcacagttccccgccctctagtggtagttacaataatgccattttgtagtc |
| | cctgtacaggaaatgcctcttcttacttcagttaccagaatccttttacaggaagttaggtgtggtctttgaaggagaattaa |
| | aaaaaaaaaaaaaaaaaaaaaaaaaagatttttttttttttaaagcatgatggaattttagctgcagtcttcttggtgccagct |
| | tatcaatcccaaactctgggtgtaaaagattctacaggggtaatgttttattattcttattatgcttattctctgtgatgcttctct |
| | acctttacagtagtagaatccttggggaaatctgcagagggaccactttcattttgaagctgctggctgcatgttttagcat |
| | gtctcttctattagagaatccaggcatggcagtttcctcccccagtgtgcaaggaccatcttcatgcctatgtctgtcgcta |
| | ggcatgagggtctctaggaatgggtgaaaaaaatgagggatgttttggaggcactataatactggggagggcagtctg |
| | ctagctggtagctgaaaggtcctggtttacttcaacattttttttaaataaaactgtgcagtagtttttgttattttaggtttccct |
| | ctgttttatctggtgtatgctgcagaagtgaactgcataacacatttcactcttagaaaatgcatttcatatatttaaatgacaa |
| | ctctgactcctatagtatgggggtgaaatagattgattttttgaaaagatggaaaaaatagattaagtgtcatttaggagtca |
| | agatgttgattgtattgtttcaaatagcatagaaactcctgccactggttcagaggtaacatttggaatgactgatgtgctctt |
| | ttgagttatctactttagtctggtgaaatatgatttgaataccagtaaaaatctatttaggttattgcaaagactcatataataa |
| | gagtttgcttgaaaagataaactatcgtaaacttattattttttctcaagtccaaattctgatatggacactaagactgtgatgc |
| | tacgtttttatgaaatgggcataatttgaggaaattggctatctctacagtatttcctaaggtactgcaaatgtgttaatttaag |
| | aagcaattacactagctagcagcaggttgctaagccagggggtccgagctacagacctctgatgtggatgacttcctgc |
| | ccggtttggcaggagctgggtcccttcctgtgcagcttccgagcagttccaaacacaaccaggcatcttctgctccttcc |
| | cactccctttctttcaagcaggatctacctatgtttccatagcctggcattttaggatttccagagatttttacccagtgaattg |
| | acttggaataaactcttaaatgtgataaagtgagccaaagggcacgtgcaaagacttggagagatatgtgcaagatcct |
| | cagctcaggttctacaatttaatatgatgtttcatctcttgttttggatctctgctattttttcagaaagaatgctgaaaaagatg |
| | gctgccaaaatattcacacaagtgaaacagcaggcacaaaccagtatcctccatttctgagctctgaattgcatattttccc |
| | acaagtagcacaatcacttaaaacattttgggagtgggagattagattacctcatagacacctactgaatgtgagagata |
| | gatctccatgtgagcaaatggatgcacatatgagattccctgaataccaaatacagctttctttgaaaatattatagctatct |
| | gagtttctatgctgtatctcagtgctcagtgtcatgtaactgacacaatcaccttttattctaatggtcattgctttttttccctcc |
| | ctgtttctgtagcactttcttatgcaaggagctaaacagtgattaaaggagcaggatgaaaagatggcacagtcagtgct |
| | ggtaccgccaggacctgacagcttccgcttctttaccagggaatcccttgctgctattgaacaacgcattgcagaagaga |
| | aagctaagagacccaaacaggaacgcaaggatgaggatgatgaaaatggcccaaagccaaacagtgacttggaagc |
| | aggaaaatctcttccatttatttatggagacattcctccagagatggtgtcagtgcccctggaggatctggacccctactat |
| | atcaataagaaagtgagttcttagtcaagttgccttcactgcctatttactaattggttctgggctagtcccagggatgatgg |
| | tgaagaaggctggcctccttccctctgtctaaagtatcactaagatgctggatgggcctgaccgtgtaatggaccaatga |
| | tcctagaagtcttttggaagcactcatttgaacctgcatttgtgagacaggcagagaactggtgaggcatcctccagcgc |
| | gggaattaaggaaggacaaaagcctattcaccttcttgaatacaaattatatgcttaaaccagtgtaaattgaccctgattc |
| | cctaataatgttgagaagcaaaaactgtaaactaggagtctatttaaattttattttttatatttgcaggagtagtatctaaattc |
| | ctctttatagtctctagctctccataagtcactttgatcttcagtgggtttaattattcctttataccatactttctcctttctattgct |
| | ctccacagaaggaataatagcaggtgacttgtaggtgccaaataagattctgagcaaagaacacacctggaaaaccttg |
| | aagttctcatgagaaaattttctaaccaaaaaaaaaaatcaaagcctcaattttgtgctttatgtgaattataaatgcggtttta |
| | aaatacttacattaaaacttgataaagttgctaagaattcctatggcattgatcacaaattttcttaataatcctcatgtcatttat |
| | caaatttaggaaagtttatagtgctcagaaaaaaaaagcatctatcttcatgtcatatgatggtaattattatgttatacactat |
| | tttacagggcaatatttataaataatggttttacttttctcttaaaatattcttaatatatattctaagttttattttatgtgttgtgtt |
| | ttctttttcagacgtttatagtattgaataaagggaaagcaatctctcgattcagtgccacccctgccctttacattttaactccct |
| | tcaaccctattagaaaattagctattaagattttggtacattcatatcctttttcaaatcgtcacttaatatgattttcttctttgac |
| | caagttattgagctacacattttccaaaatatctgtggttggcaatgttatgtgttctttctttttctttccttttactcaatcgttag |
| | catgttgcaaaatgagatcacaggtaagtgaattactttcccccgtcttctaagtgtttcttctctacccaactcactattactt |
| | ctttcttctcttttcttctcccttacgaattgcttgccacatcccaagcctttctcattaattttgaccatgttaccaggctttcctc |
| | ctgtaagtcttcaatttacaatgttaggtaagggagtaaactccatgagctaattttctttactgcttttacatttggaaaataa |
| | atatacataatctgaattacaattttgtatgtttttggtctgaattttatgactttcttctattttagcatttaaaagctttgagttagt |
| | aagcgtttacaattgtgtctgtaggtataacaccctttcaaatacttttccaaatttgtttcgcaacagccttcttattgatctttt |
| | gctttctattcttttcctttctttttctttttatcacttgttcctatgttttattgaagtcacaagtcttgctacaattatccctctcc |
| | aaaggattaatgtctatctatccatatacattctcattttattttttatactccttattgaagcacctgctttccaaaaatgagattga |
| | tgacatcttggtgggagatggcaatttgattgattccttgaaattaaatagagtagttggaaatgagagattttattctaggcca |
| | gaaacaagtcttgttgacagccagtcttgggaatgccaatgaagcaaaggcttggaagactgaagctgtgtggggcag |
| | gggcatttacacgaagaacacagaagtcatgggaggaggagtgatatgcttcaggaatcagaggtaaggcagggga |
| | actgaaattaagcaaatcctcagattaggacagcatgttatttcttcttcagaaagaatcgttgcttggaattccatgatgtg |
| | gttagcccagggcctggtattaaggctttcagtataaatattctccacctttagcaggcctagaaaatatttgaattagataa |
| | ggtatggaactaaattaagtagggaattcaggaaatgggataagcctggtacagggtacttatgtcatttctgggtggag |
| | gggatgtaaagcatgtccctagacttgcctcccagtatggcaaatgttgtctttagaagtgtaacattctgtaaagtctcctt |
| | ttaacctctaggttgttcctcttccccagtttaggtggatatcaacatcttttatttgatgtttatgtttcatgttttaagatttccta |
| | gtttcttggcattaccttaagcaataatgttttcttacctctctctgttttccaaataagagaacccagtagcatgtggggaaa |
| | aagatgtctttagttagcattagaaataaataataaagttggaatttatatttgggtctcatgattataaattatgatctattatt |
| | atgtttcaagcatttgtaatctgtgcagtgaataaatctctgcataaactattcattatattttaaaataattgtatgttccttatgc |
| | aaacgtaatttatatataaaattacgtggaaaattctagcctagaactagacttctgttcctagtagacattgggaaaatattc |
| | attaaataaataagtgactagtaagtcagagattagagaatcagatacaaaaaaagtgaaaaaataagtttgaatggatc |
| | agaaaaaatctttcttgtcgtgcatctgaatgatgagatggagttaagaaaacccaatatatttgttttttacaaaagcagatt |
| | tttgttttaaaacttttgtaatagaccatggaaaatctcatgaaaactattgtccccacttgaaaaaaaaaatcctaggagatt |
| | atgaatccccatttaaaactccctggaaaagagactcctggtggtagagggtaagggcagtttaagaaattctgatcaga |
| | gaatatgagtactaaggacacagtttgtgccaggacctctctagatatccagatacaacttgaatctgtgggcttgtatttg |
| | cttcctgggggaaagaactcaccctccacaggctgaagtcattgggaggcctgaggagatgcaggttgaaatgtgaag |
| | ccagccaggcaaatctattaaggtatgccaagttgagctgttgattctctgagaagtttacagatggcttagagtcacaaa |
| | tttattccaatgtagaaaattagattttaaaaagtctctaatttcctgacttaaagtgttatatttcagatgtctcaccttggagc |
| | agagatataacaaaaggcagtgaggctaatagtctaagatacatgaatccttccatgttttggtgatgctggtgcaattgat |
| | caaatagcccagtaaagttagaggtatatagatgctgtagttagtaactgattttcacaataattttgtcctttattcctcttgtt |
| | gcaaaccctagacttaaatcctgattttctgacttcaagtacagtgtcttttactgtaagttaaaaatgcttggagagatggtc |
| | atggttgtttggccacagttgggaggtcattgtatatttattaccactagtttataaaccaacaaggagccattcatgttaaat |
| | aagtttttattttaaacttggactaatacctctatttcaaacaaaaaccttgacttgtttctcaaagagctgttatctattaggag |
| | ctattgtgtatcaaattagcttttttaaaaatttattttggctgaatgagaaattatgcttgtgatatttttaccagggtgcattctg |
| | aaaactgaaaattcttttgatgtgcctagtgtcttatttgatatttaaataaaacatgatttattttctagataacaaacaagttaa |
| | aaataatctatgttcctaaagttccctaccaagcttttaaatgtgtttcctgtcagcttttattattttaagttaatatatgcacact |
| | cctctaatttattttgcatttgttactcatttgttcatttgcaagtacttactgagtatctaccatgtggtagatattcttgtaagcat |
| | tgggatgcaacattgaacaaagtgaagttcctactctcatgtagttacattcatgtgagtgtgtgtgtgtgtttggaagaag |
| | aaagacaataaacaaatacgtcgattgggagctagtgataagtgttactaagaaatataaattagtgtcaaagggagga |
| | gtgacaaggtgttgttttagattgtatggccagtaaaatcctttctgaaaaggtaccagttgagcagagatccgaaggaag |
| | caaaagagtgagatatgggaatctagggataaagtcagttcaaggagaacagcaagtatgaaggtaaagtctgaaggt |
| | ggtggtgcaactagtatgtttaacaatcagcaaggagacctgagtggctggagcagagtgggaaaggcaggaagcg |
| | agaggatgacatcagtgggagtgaaggtcaggggctaaagttgtaggcgaggtgcagtgggtcattgtgaggacacc |
| | acatttactctgaatgagactccaggagggttttcagtagcaggatatcatgacttgactcacatatttaaaagatcactctg |
| | gctgcttcatggagaatagactgcataaggggaaagagtggaagcaaggagactgggaccgattgtcattatgaagg |
| | caagagagcatggtggcttggagtatggtggaaacaatgaggatggtaaaatgtcatcaaattttgaggtattttgcaga |
| | agagctgacaggatttgctgagcaattggttgtggtgtataggaggaaggcagggactaaggatgattccaagcattta |
| | gtctggtcaagaaagaaaaatggagttgtgttgactgagcttggggcaacttgagcaaaccaatttggtgggtaagatca |
| | agatcttggttttgaacatgttatgtttgagacacctattagacatcattggaggagttgaggagttaggtggctgtgcaaat |
| | ctggagttcagaaaagggctgggctggaggtgttcaactttgggagctgtcagtgtacagctggtatttaaagtcatgac |
| | attggactaggctgccaaagagctgagaccctccctccaaatcacactagtaatgctgaactacctatcatttgaaaatg |
| | gtaggaaaatggaaaacataggttttggtatcagaaaacgtagattcaaaccctatctctaaaatttactttttagctatatga |
| | tcttagtccaagttactccaattctttcgaatctcagtttctctatctgtaaaattataatcacagcttagacattaataatgataa |
| | aatgtatgacaagtatctagcaccagatcccatgctagtacttagtaggtactcaataaaggatatctatgacagtaatagc |
| | taaaattctagcagcaactgctgtaagattagcaaaaaggaaactctcatattccttaaggaattgcacaaagaactttata |
| | gaaatccctactctgactctgcaaacaaaatctttatatagcaccagagtttagacctgcaactgacccaaacaatgtggt |
| | cagttctgtctcattttgtagatgagttcactgaaacccagagatatttagttttttctaaggctacattttctatcagtggcaga |
| | gctaaaacttcagaccaggttttttgattcttggctctttgcattttgcatccaatagaaaacaaatgatttttaaaccctcgga |
| | tttaatatacttggggcattgccagtgttcttgttttatgcatttcaaaggtgcttcttagttgctccaacttactgattcattaaat |
| | agtgtccatactgagatataaaatatcatggttttccatgaaaagaaatatacaggtttatatgaaagcagatgacacaaca |
| | atttctctttcttttgttttcaatgctcatatgttatcatttagttatctactggcaaataggagtttgttcatattaaaattaaacaat |
| | ccaatatttaacactgtatatgtgacatttactcgatttttctgctggctcagaaatatgcactggtatgcagaaaaagaccta |
| | ttctattctacttcaaattatccatttttacattagaaaacctctaacatcaggctatcttctacttctagtttatatataggttaaa |
| | aactcctctgcaacttctctggatattatacattattacaaagtctctgaacagagcataatgtcttttccttcctatagaataa |
| | caaagaaatgtcctataattttatactctataaatgagttattaatggtaagaaaccaataattattatcttagtggataatgac |
| | tgtatactgtaagaaaagtattatccacatttatataagaaaactgagcctcaaagaattaaacaaattgctgaagcccaca |
| | tggctggtaagggatgtatctgaccatggttcattgctctaaatctcatggtgcttcatcctcgctccacggagacagggg |
| | tgggtgtgccagtgttatgatgatccaggctccatgtcaagggctacttaaacaattttcactaaaaacttgaagaagtgttt |
| | cttcataatatacacaaaggaaatattttacatttgccaactcgcaggttagtatcaatcaacaggtttacccactgttatgta |
| | tacctggcataaagaaattaatagattaaaaaacatctttgtcccctgatattataaaaggtttatctgcctctattttattttac |
| | attgaaaagttcttaaagcaatattgttccaggatacagtgttcttttgaaaaatgtactctatgacttggattacacatttaaa |
| | aaataatataggatgtatgcattttgctactagtttgagccttttgaaatctgctttgacgtggggtttctatacttttttgatgca |
| | tggcatcaccaatgcaaaatccatacctacattaaatacttttgtatttgagtttttgttatttgagtttttttttttttttttttttt |
| | tgagacgggttctcgctctgtcgccctggctggagtgcagcagcgcaatctcggctcactgcaagctccgccttccgggttca |
| | cgccattctcctgcctcagcctccggagtagctgggactacaggcgcccgccaccacgcccggctgattttttgtaatttt |
| | agtagagacggtatttcaccgtgttagccagggtggtctcgatctcctgacctcgtgatccgcccgcctcggcctcccaa |
| | agtgctgggattacaggcgtgagccaccgcgcctggccatatttgagtatttttaagatcatctgaaactatttcagtcact |
| | caccagaatccaggaatttgtaaagtatgtgactgatgaaataaattaacaatgatttagaaacttagtgaattttaagccttt |
| | ctatttagagatacctatcaaaccacaagcgtaaaaacttgaccctagttatctactatttttctattaaaagcaaaattgttct |
| | ttttatgtatcagaagttttaacttaagtgtatacttttattaaaatgatagccatgaaataaggaaaatgcctgttttcgacttat |
| | tatcagtgactaattagaaaataattatttctcttgttaatgttgaaatatatattttacttttttatatataactaaattataccacta |
| | taaagagtaagtttttaagtgtcataaaaccattgccgagtccataatgcagcataattgcataaggctgttaatttccacctt |
| | atatttttcttatatttttaccctcaaaaaatgtagaaacttgtgtaaacaatatgtatatatattttagacagagtctcactctgtc |
| | acccaggctggagtgcaatggcgtgatcttggctcactgctacctctgcctcctgggttcaagtgattcccctgccttagc |
| | ctcctgattagctgggattacaggcatccgtcacgcctggctaatttttgtatttttagtagagacagagtttcaccattttac |
| | ccgggctggtctcaaactcctaacctcaagtgatctgcctgcctcagcctcctgaaatgctgagattataggcgagagcc |
| | atggcacctggccaacaatatatttgaagacaaactttatgctgtatttttaaataatttatcagaaattgtttttaaaaactcca |
| | tttagtaacaaatgaattgcaaaattaatttcattagtcaactgacactgtgaaatagcaaggctataatggtgaataatata |
| | gacatgattcctgtcctcatgacgctcagagagtagttgagaagatcatcattaaaatttgtcattagaggaataatataag |
| | ggctgctggggtatataactggtaataggtcaatctgagtactaaagaaaaagagaggtgacatttccaagaccctaaa |
| | gtcagaaacagcatataaaacattcataacatttgacaacctaaaataattacagtattatccaaatggggataatgcaatg |
| | aagagaaaagaaaatgaaactggagaagtagacagggatcagacctcctagcgtcttgactctgtgttaagacatttga |
| | tcatcatcctaagagtaatagaaagctaccaaaatgatgcatattacatttacaatggtcgtgttagcacagtatgcagaat |
| | ggattaaatggagccaaacatgaatttggaaacatcagtttagaggagactgcaataatctagatggactattagatttga |
| | tgctagtcttgacagacacttggaccatgcagtgatatggggatggaaaaaagtaactgaatccagagatagcaggca |
| | gaattgacaatatttgatggttaattagatatgaacgtttaaggggagacagaaatctaagatttctcataggtctctggcta |
| | tattatgcacattttataagacacagagacgtcaaaggagtaagtaattagcaggaatggaggggtagattaaaagatac |
| | ttttcaaaagttcagttttagaattcaaaatttgaagtgttgataagatatgtaagtacagatgtcctatggacaatcaagtat |
| | gtggaattcagaagagcggtctcacttggagagaagtatctgagaatggtgggtatataatggttatgtttgttgagcaat |
| | gtttgttgatggactacactaggatgaggagagtagaagagaggtagatggcttacataccttatgtctttcttttcaaaaa |
| | gaaaaatgccacatttcaaagaatacagagaaattggtgccctgaggcatgaagaagccagggaattggaacctcctg |
| | aaagcagagggaagataacttaatgctaaggggaggaaccatccttgttgaatgctgcttagaaagcatgtcaaatata |
| | actcaaattatccttttgtttttagtgacaaaacgtgaagatgttgccatcttttagaaagaaaagctggtaggctgaatatat |
| | gatagatatataagaaaaggaaagataagcagctctttcaaaaagtttggccatgatatggaaaagggagataaggctg |
| | tagtagttaaggagggttgtgagtcataggagagaaatatcctttcccctatgccaggacaagggagacgtaagcaagt |
| | ttgtgttgctgggaaaaagccagtaaggtgggagccattaaggataagaaaagagagagataagagattgctgaagtc |
| | ttctggaatggggaaggtgtccagaataccggtggagggattggaaacctccccactgtaacaggaaggaggaaaga |
| | attggtctcaatgtggaaaagtttgttgatttggggggggaagtggaggcggggcattgtgatgctgtctcttctctgtaa |
| | agtagaaaataagttttcagcttgaaatggagccggaagaaagaagagggttggaagctggaggaaagtggagaata |
| | tttgaaatttttctttgcagagagtgggagatggagcctagtaggaaaatacaggactgtgttgaggaccactgaggtttg |
| | tgaccataaatttagaatggtgccaatctgccacggggtgttatttttccccaatagggctcagcagaccaacaagcaca |
| | ggggaccctctagttttatataccaatagcaagtcattctttatttaatttagttttttgtttgttatagcaataaagaaaaattgt |
| | gtttctttgaaatggtattttgtgtgtgtgtgtgtgtgtgtgtgtgtgtgtgtgtgtgtgtgtgtgtgttctctagattatagtgcc |
| | ctcacgtggctgatgcacatcatttacttatgagtgttttcatatggatgaaaaaactgagaattaagcatgggccttacag |
| | cacagctactaaaattaaaaaataataattattattattgttttgactaaaaccagtactagatgcaaatgaactttctttctag |
| | atcaacacaacattgtccagttgtaatagtgttgatatttcattatgtgtaagtaatgtgatcatttatagtaaaaacattagga |
| | gtgagaaaagatatgaagagcacgtatttcctctctggaatttctataattgtgtccagattcacaatgataaagagtgcca |
| | ctttacataatggcaactaaatctttattatgctttttattaaatatgaaagtcattactattatctgatactgaatattttcttaaat |
| | agttgttttggtttttgtcttttgctgttttttaatgtcaaggaaagtgaagggcattggctatacctgcaaagacaaagtggg |
| | atagggtgaaaccaacccaatatttgtaataaactgtgttctgtatgatccaggaagcaattcattgagcatctattacgca |
| | ctagggccattaagttgaaggagacttacatattttaacaaatttgatcttcatagcaatcctgttacatgattactttttcttac |
| | ttttataaccaagtcaattgagattcagggaacttaataagtaattttgccttagttacaaaccccagaaatgccagtgctaa |
| | cacctgccatcttctctcatagttcaggattttatgagcaattacagtatattataccctctgtttagaaaggaccttattataa |
| | gacattccaccagggtaacttttagaatgatgttataatacatttaattaattacttgaattgtcttgttgaatttttgccagggtt |
| | tacatatgtgctgaacttgcagttttaatgttcagttgagtctgtcgttaagaaaatttaagttgataaattattcactgatgaac |
| | tactttctttgcatttaatctttttaattgctaaaggtacctaaatagcctcaaaatagttgatggcttggcctgaagacaagat |
| | ctaaatatgaggttgctgagttatagaaatggcaaaaaaaagggtcaataatagaataataagcaacaaaataatagtaa |
| | gcactaaagttttaaacttcatggtggtgaaggcatggtagtgcataaaagtaagatttttccattgaactttgtcttccttga |
| | cgatattctactttattcaatatgctcattatgtgcacgattcttaccaactgtgtatttatgaccatgagtaaccctccagact |
| | ggacaaagaatgtggagtaagtataaatatttttcaatattgacctccctttagtttcatattgtgcttttaacaccttgagac |
| | ctcctcaatttctttaacaaatcatgctagctactgttaaccagaccctgattcaaattcatttctgtcactaaatgtcttctagg |
| | acaaagcttgtagtgggctcacttagttgtgtaaattactgcagtagtttgactgctattatctgcagccctttatcttctttgtg |
| | agtcttatgttcttttgaagatcaccagtgatttactaatatctactgataaaaagtatacctagtttttatgttccttttttaatga |
| | ctaccacagttctgtgttactagtatatgtttgatggcttttaatggtgcatattttatgaaatacaaatgcttcactcatttttgta |
| | ttaatacctatttgctcaaatcggactgaatgccagtgtatttcagattatgtcttcatatagagccacattatttggatccttta |
| | aattaattaatgtggaaaatgcaatatacatttatttacagtcaatgagaatgtcttttggaatttaatgtttcttttttgacttaag |
| | ccccacctaaactctatatcgtagggggaccaacctggaagtgtctaatttttgtttgctgtttatgtcatctttaagatatgta |
| | cttgtaaattaaccactagatttttaatgtgagcttggctattttctctcaggtatacctttacaggaatttatacttttgaatcact |
| | tattaaaatacttgcaaggggcttttgtttagaagatttcacatttttacgggatccatggaattggttggatttcacagtcatt |
| | acttttgcgtaagtatcttaatacattttctatcctggaagagtaaatcactggtgggagcctatactatattttccttggtggc |
| | ttgccttgacagaccaagcatttttcttagtaatcatagttttcttccaatcaaattatccagtttggagaaattaggaactatc |
| | atagtaaattacatggctttggtttcaattagcactgtaaagtaataaagtttcccaaataacagagattatgattgatgacaa |
| | tgccattttcctcttaattgggaaagctgatggcgacactcatgaaattaaaaaggtcttgatgaaagaccaaggaagac |
| | gtagatttccctaaattctgaataactctgatttaattctacaggtatgtaacagaatttgtaaacctaggcaatgtttcagctc |
| | ttcgaactttcagagtcttgagagctttgaaaactatttctgtaattccaggtaagaagaaaatggtataaggtggtaggcc |
| | ccttatatctccaactgtttcttgtgttctgtcattgtgtttgtgtgtgaaccccctattacagatatgtgacagagtttgtggac |
| | ctgggcaatgtctcagcgttgagaacattcagagttctccgagcattgaaaacaatttcagtcattccaggtgagagctag |
| | gttaaacaccgaggctgactttagctacagtggtgctacaatcacagcttttgtgcagaagccttgttgctagttgcatatt |
| | gcaaataaatatgtaaaaaagcaagaattggtacatcattttttggatggatttgattctttgctttttactcgttgctttctttaa |
| | aactattctaaatcagcctttgagtttaacaagtgttgcatgaggcatttgcagtaacaggctacatggtttgcatcctataa |
| | catcaagctttccgcatagaagctagactaagagacattcagactgatgcaaatttgacagtttaggcctaaaactggca |
| | atcttttaagctgcagataaatgaaagagcaagggatagcatgagtgctgcatggggctcagatttcagatgtcttcctttt |
| | tttaacccatactcaagcttgcagaattcacaaatatataacctcataattcatcgacttcaagatttcttactactctattcac |
| | atagacttttctaaaaccaataaggggttagggagtaagacatctgcaaataaaagcaaaatatttacacaaggttgatgt |
| | ttaagcatgaataacaaaatcattcttttgctctaaagagtgtttggaaatacacatttggttcatttccattcacagttttctaat |
| | gaacatacaagttctgctttcattcattttcaccagctagcaggcttttcatgaaaatgttattcaatcacaaacattaaactaa |
| | tattgttggcattctgcatgacatttttattttccaggacaagctcatgatatttttgccggtaaaatagctgttgagtagtatatt |
| | taaattcccccttctgattttgtttgtaggcctgaagaccattgtgggggccctgatccagtcagtgaagaagctttctgatg |
| | tcatgatcttgactgtgttctgtctaagcgtgtttgcgctaataggattgcagttgttcatgggcaacctacgaaataaatgtt |
| | tgcaatggcctccagataattcttcctttgaaataaatatcacttccttctttaacaattcattggatgggaatggtactactttc |
| | aataggacagtgagcatatttaactgggatgaatatattgaggataaaagtaagatatactctataaaccattaagttgttta |
| | gttctctaaatattaaatattatatataatggaaattatctcaatttagatgtgaatcaagtgacttagactaatttaagatgattt |
| | aatacatataaaagagatatcaaatgataccttattctatttttcttatctgtccattgatatagtaaaagttctcatttgaaaatg |
| | tgttgtcttatactcatgttgaaagtaatttcatattatgccatattaaaaaatgtttatttggtagacattaatcaggtttttcagt |
| | cattttaataaataagtcagtagtttgaactattcagtgtattccactgaaatgtgttaagaagactgaggggaaataatttg |
| | gccctatttggttgatgcaacatatgtattgagtacatatgctatatctgaaaatagagaaaccatttatcaagatgaaataa |
| | gaatttgtgtgctcctcagaaggttaagtaaccctgatttagccattcacttattcatattctaattagtccctttagtgtcatca |
| | ttgtattgtagttaccagtttagtttgattatatttaaggtatgaacatcagaataagcttatgccatatacttcagcatgatttct |
| | taacattgagcccagcccctctgtcatttttcatatgtgtgtgcatgtttgtatgtgaatataaaaatacgtatgtttgcatgtgt |
| | gtgcatgttttctgagatcatctttgcaacttactgaagttatatgtcatgccttaaaaataaaaactagatagctctccatag |
| | cttaaaaataaaaactagatatactcagacaacatatctctccaaagaaacaagtttattttcttcatttgaaaggcagaaat |
| | caagcaaaaatttcaaacaaaacacttatttacagtatcataagagggaataaatacctaatcccacttctcacaggaaatt |
| | aagttaaaattggcgggaaaaaatgtctgaatctattttgagcctggggagaaaagtatatgtaaggtaaaatttatttgca |
| | tgaaaacacctagaaacaacaaggctttcttctttcttactttttgtgcccagcaatagactggcagctctttcttaatgtatcc |
| | catgcaatttgagcttatatttgcaatgaatgctgatataagaatgttatcatagtaattccttctgaacatttttctttttaacata |
| | gatttgctaaccatttgtataatcaaaaatgttatatattgatatttgttcaatattgtgaaaaatctctttagccatatatatttatt |
| | agtttatccatctcattatgattgaaaacatttgtgagctttgccacctaaacagggggctgaagtgttttacaggattttaat |
| | gattctttctattcctttctctttaaataggtcacttttattttttagaggggcaaaatgatgctctgctttgtggcaacagctcag |
| | atgcagggtaagtgatgcttcctactgagtttcagtccacactgctccatcagtgtcaataacctgccacctcccactcatc |
| | cagtcccactcactcctcactcaaaaccctccataaattctacttcacggtgactctcagaatagccaggataagtgtaga |
| | ttctcacctctttcacacagtcatttactgcaattatttttctatgctaggtcacatctaatcttccaaattagttcaatgtaaaat |
| | agagaataaagcagtataatatgcatctgaagcttaatagaattcttaagcacatactttttataagtgtcatattttatatata |
| | ctaatgtgttctccatagcttaaaatgtaagatctctgaaaataatgttaatatctgagacatggggagtatttagcatatttta |
| | gcaaagtggttacaaacataaactggagagtctgcatagagtcagactttgactccatcatataatctcatttcttcttgcctt |
| | cgtttccttatatgacaaatgggtataataatagggtttttgtgatgatgaagtggattaataaatgtatagctatttagatcgc |
| | tcaataagtgcttgtaattgttattattgggatcatgcaaatgtttgctattaagaaacatggagctaaatcctaggaaaattt |
| | aaaaacacagttaattttctttatttagcaagattttagagccacacacaaaagtctaatgcactttctttggacgatgatact |
| | gtggacattagtagctaatacctgtagcaaaattcccagtgataataggctttccatttggctcctacgatcagtgctatgct |
| | gcctttatcttcagattccaatgataagtaaatcaattgattttcattccttgtttgtactgtactaaatgcgttacatacagtatc |
| | ttcttcaatgtttgcaaatttgtgagacaggttctcttattagcccattctcacatgcgaggtgcctgaagattagcaagttaa |
| | gtaacttgcccaagatcgttcagctcagaagtgtcaggcaagacattgaagccaggtctgcttgatcttcaaggtcctcct |
| | atgacatttttaccacacagtgtcattcactccttgcagcatgccccacctatccttttctcacttctttaccctgttcccacact |
| | tacacacatttctgcctcaagacatcctcagtgaaaatcaactttttccttacagacttttttaactgcccttaagtcccagaa |
| | gatattaatcatgatatgattgcttttatatggagacataataaatataataatgacaattatgaatcacagaggaatccaca |
| | aagtagaccttatagattctgttattatataaatcagtccacttaggctgagttaagtactgggtaaggtgagagaaatcg |
| | gcttttttctagtgcctgtataaaacagacattggcatatattaaaacaggaaaaccaattagcagacttgccgttattgactt |
| | cctttctttcctctaacctaattatagccagtgtcctgaaggatacatctgtgtgaaggctggtagaaaccccaactatggct |
| | acacgagctttgacacctttagttgggcctttttgtccttatttcgtctcatgactcaagacttctgggaaaacctttatcaact |
| | ggtgagaacagataaaatcatttttctgagaatcataaaacaccgaactcaagagaattgctgtagaatattttattacttag |
| | agtgtaagtttgtaacatcctatataaaatttattaaaatctctcttccattttgcagacactacgtgctgctgggaaaacgta |
| | catgatattttttgtgctggtcattttcttgggctcattctatctaataaatttgatcttggctgtggtggccatggcctatgagg |
| | aacagaatcaggccacattggaagaggctgaacagaaggaagctgaatttcagcagatgctcgaacagttgaaaaag |
| | caacaagaagaagctcaggtatagtgaacaagcatacggtcctttgtttttctttatctaaattctttaacctaaatgttgagg |
| | tcagtggcaaggtagttgacattagaaataggtcatatgtgtttggtaagtgctaggagcctgtttggttattaagaagttat |
| | tactttattgcaatgatctctgtcaatagtgtcaatagtaatggcatcaaaaaatggataattataattgctttactgacatttttt |
| | tctcccttgtgactccttgaggaaattaatgattaacaaaggcctcatgtactcaaacttgcagagtagataaacctacatg |
| | tcctcagttgaagtattttcttaggggaagaggaattcagttacacttgcttcttcattgcagtatcaccagaggtggtaagg |
| | gtcagaaaaccagaatcaaactaagaaaattatttcattgagtctggaaaggcaaaggcttattcaatatttgttctcttttat |
| | ataaagtgtacaaatgcaagtttgtgggttacatcagtaaatcactagtgtgtaaacatattaaaacattagcactctctgcc |
| | tcctactctacaaatcctttaatttggacttgacaagccttcaaaataaggcaagaatttctctaattatatttgcttgacttaat |
| | ggcattaactaatccaattgcctatttttgtcttttcatgtatggtgaatacaattcccttttattaccgagtattcctaaatatgta |
| | ataaaggtcaaagtatattgctgtaatagcaacaaaactactgttatactttacaagttcatgcagatgccatgatctaggat |
| | tctcaaataaacactctgtattatgtctttgctgtgcatttcttagtgaaatacccaatttaaatcacggagaaaaatgtcatta |
| | aaataaaatacttgactgaattacatttaataattcagactagcactaaattttttattgtgtgaaaatggaatcaaaggcaa |
| | atgtctaccaggtttaaataggaagtctttaattcccatattatttccttcttaaaatattgtttgaattatagaacatgttattatg |
| | atctttaagtgtcttgctcatattattagataattagatatcatagtgtgaggacagagcttgaaggttctcataaaagtcgtat |
| | gtatcatcttccatatgaatgcccattttactctttgattggtctaataacaatgtactgttttctaaaacacagaataaaatgg |
| | agaattgtttttcaagattatcttcatgatattgaagctcaattaagcagtaacatgataattactttttaagtttatatgcaactt |
| | ccacatactttgcgcccttctaggcggcagctgcagccgcatctgctgaatcaagagacttcagtggtgctggtgggat |
| | aggagttttttcagagagttcttcagtagcatctaagttgagctccaaaagtgaaaaagagctgaaaaacagaagaaaga |
| | aaaagaaacagaaagaacagtctggagaagaagagaaaaatgacagagtccgaaaatcggaatctgaagacagcat |
| | aagaagaaaaggtttccgttttttccttggaaggaagtaggctgacatatgaaaagagattttcttctccacaccaggtaaa |
| | aatattaaattacatgaattgtgttctcataaattttttaaaaaaatatgccagaatttaatggagagaaaaccgccttccacc |
| | tggatggcacaatgctttcagagtagtgatgattatcaagtgttttggctatcacttcagagaatttgtgagttttgcaactttt |
| | tggaatcccaggaaggaaattttagatccctctgggtttggaaaaatttgcggttttgaggttttcttaaagactgaaaaatc |
| | ttggagaaattttccacatcaggaattatcagcagatggttcccatctcttcttaactattgtgcgtggatctagtgaactttg |
| | ggttttctgagtgacaaattcccagaagtggaccagagactcttttaggccacctgcggggttgttcccataaggtgcaa |
| | acatcacttgccaagtgcattcttcatgcctttgtttcaaaggggactgaaacaaaatatctctaaaagtagccaaaactct |
| | cagataggcaggtactgagggagatttatgacacgaaataaaaagtggtgtttagttgtacttgattatctgtgtttcatgtt |
| | aaacatgggacttgcatttgaagaatactgtgatttataaactgcaacaaatattcactggatgcctctgccttttgtactcat |
| | gcaagttgttgaaattttaaaatttagaatcttaattgtctttgaaattaccaagagaattcacaggaatacacagtacctcag |
| | aagacattttcaccaggagtgaaaccttaatacctatacagtaacaataacaattacaacaacaacattgataatggctaat |
| | atttatacactgtgttgttatttgcaatattaatgcattaagcctttcactgcaaccctaggtaagttctaatttagttaatatccc |
| | caatttttatatgtgaagagaagcagggggctgcattctttgttgagtacctattttatgcttggcattctttctacattctcata |
| | attaatgctaacagttctgtagaatagtattattttcattactataatgaaaaagctattttggatcagtaaagttaaatagttgc |
| | acaatgtcatatagcaatagaaaaagttattatgatctttgagtgtcttgttcatattagggttgttggtattcaaattttgcctg |
| | gtactgaaaaccttaaagtttccactttatcaagttgcctatgaagaatgcctttaaaaactgataaggaaatttacaatataa |
| | ctttatttaaaatacacaatggcattatactttctcttttacctttttataatatagacaagctcacataacctcacatgtgatatat |
| | ataaatttttttaggtcagccttattcatttcaaatccaaataacatcataagattgcatacttggggattaattcaaatttaaca |
| | taggatctttaaatatcaaaatttacttggtctctttcattttgttgtcacaatcatgattccattagtagaaacattaatcaataa |
| | ataggaatcctttaaaaggcaaaccccctgtttacagtattagtcattctgaaaaggaaggaagaaaaagaaagggagg |
| | gagggaggggggagagagagagagaggaaaaaagggagcaaaggagggaggaagatagagtatttttgcctaca |
| | tttttacctaagtttgtctgaatttttgcctgaagttgtctaagttttggccaaaatttgcctaaattttggcctagatttgaaactt |
| | catatcaacttcatatcaaacacttaccacagagattctcttcaatttgccttatttctaattgaataaaactaattctaggcaa |
| | aatagtgaagcctgataaggctaggctctgtccttcctttttctgtacattttgttcatagatatgatattctcccagcagcctc |
| | ttcttcatacctctacatacctccatttcccagctagttgggtattataagcaatcactgacttagaagacatggcatggctg |
| | gctcatattgatacttgtttcttaagcagtctctatataaaaatagagttaaagactttattttgcttgataaagaaatagtcaaa |
| | caaatgtctaagaggatggagagggagacagaaaaagacagagggaaagggaaagaaagagaaagagagaggg |
| | gggaaaggagaaaaggaagaagagaggagagacagaaaaccctgaaatcacaccaaccccacttggcaagccct |
| | gaaagtaatactgaaaatgtcaaaccagatagaagtacttaatcttggtatagcaatggagggcccatcgtgtctgttaga |
| | tttttaagagtttgagaccccaaaatattaggaattattgttttgtgatgacatgattgtgttggtaaccatctctgtgtttgtatt |
| | gaaaaactatacaatgcaagcctttactgcaagattataatttctttagtagagtaagtggaaatatgaattgtttctcagact |
| | ctgatttgacttgttagtgtggtaaagaggggagaagaaagtcaagaatgtaatctctaaacaagtttcaagataatctgg |
| | atttttttgaaacctttataaggtacaattgaccttaaatcattactttattatttatttgtgataagctaggagtttaggagttttg |
| | ctttttaaagattggtttggtatggggaatatttcttactggccatcttttttgtgtgttacagcatttgattactatgcatttatgta |
| | atgaatgtcagcaaaagaagttgatgctaacggatggggcacaatcatttctcatatagctgtcacatgtaaactacgtttt |
| | tgtatagcttaattcatccatgatccttgagaaacatgcaaacttataacttattttcttccaacccttctatggctccagctga |
| | atggggtactggcagttaaaatataaactcttactaaaagcgatagaaacattcttcattgcaaagcatgtattgtttgccttt |
| | cttttttagctaatgaggagcagtatgtcacacatcctgcaaatcccgtaactgttatttcctcatagctaattcgaagtccct |
| | tgttagaggagagaaaggagacacgaaaaaggatggatagtctaagaaaggctttaaaaaataactacttgtatggaaa |
| | atgataaaagaaaagaatgaatgttactaatgtagttaataggattaaaaagcatgggaacaacaagaggagagatgac |
| | ttctgttgtgggagcagtaagtcttcttagaagtagttctaggccgggtgtggtggctcatgcctgtaatcccagcattttg |
| | ggaggccgagacgggcagatcatgagatcaggagatgagaccatcctggctaacacggtgaaaccccatctctacta |
| | aaaaatacaaaaaattagccaggcgtggtggcaggtgcctgtagtcccagctgctcgggaggctgaggcaggagaat |
| | ggtgtgaacctgggaggtggagcttgcagtgagccgagatcacgccactgcactccagcctgggtgacagagtgag |
| | actccgtctcaaaaaataaataaataaaaaaaaagaagtggttcttactgtaaataatgaatagaatcacataagatagtgt |
| | ttaacatttacagacatttaatagaaactaacagatattattgagaaaaagtaattctttagctggaaagaaaataaaaagc |
| | atacttattggtcagtgtattactctgttttcatgctgctgataaagttatacccaagactgggcaatttggcaaaggaagag |
| | gtttaattggacttacagttccatgtggctggcaaagcctcacaatcatggcagaaagcaaagaggagcaagccacac |
| | cttacatggatggtggcaagcaaagagagagtgaaagccaagcaaaagaagtttctccccatataaccaccagatctc |
| | atgagacccattcagtaccatgagaacagtatggggaaaacccctaccatgatttaactatctctgaccaggtccctccc |
| | acaacagtgggaattatgggaaatataactcaagatgagatctgggtggggacacagagaaatcatgtcattccacccc |
| | cggcccctcccaaatctcatgtcctcacatttcaaaatgaatcatgccttcccaacagtcccccaaagtcttaactcatttca |
| | gcattaattcaaaagtccacagtccaaacaaagcctcatcggagacaaggcaagtcctttccatctatgagcctgaaaaa |
| | tcaaaagcaagttagttacttcctagatacaatagggatacagacattagctaaatacagccattccaaatgggagaaatt |
| | ggccaaaacaaaggggctacatgccccatgcaagttcaaaatccagcagggcagtcaaatcttaaagctcaaaaatga |
| | tctcctttgactccatgtctcacatccaggttacgctgatgcaagaggtgggttcccttggtcttcagcagctccacccctg |
| | tcactttgcagggtacagcttccctcctgactacttccatgggctggcattgaatgtctgtggcttttccaggtacacggttc |
| | aagatgttggtggatctattattcttgggtctggaggacagtggctctcttctcacagctccagcaggcagtgccccagta |
| | gggaccctgtgttggggctctgaccccacatttcccttcctcactgccttggcagaggttctccatgagagccctgcccct |
| | gcagcaaacttctgcctgtacatccaggtgtttctatacatcctctgaaatctaggcagatgttcccaaatcccagttattga |
| | cttctgtgcactgacaggctcaacaccatgtggaagctgccaaggcttgaggcttgcaccctctgaagccatgggccta |
| | gctctacatttgcccctttcagccatggctggagccgcagagatacagggcaccaagttcctaggctgcacacagcgtg |
| | ggactctggacccggcacatgaaaccactttttcctcctaggcctccgagcctgtgacgggaggggctgctgcaaaga |
| | tctctgacatgccctggagacattttctccattgtcttggggattaacattcagctcctcattacttatgcaaatttctacagcc |
| | tgcttgaatttctcttcaggaaatgggattttcttttctgtcacattgtcaggctgcaaattttccaaacttttatgttctgcttccc |
| | tatgaaactgaatacctttagcagtacctaagtcaccacttgaatgctttgctgcttagaaatttcttctgccagatactctaa |
| | atcatctctcccaagttcaaagttccacaaatctctaggacagggccaaaatgccaccagtctctttgctaaaatataaca |
| | agagtcacctttgctccatttcccaacaagttcctcatctccatgagagaccactttagcctggaccttattgtccatattgc |
| | catcaggcttttggtcaaagccattcaataagtctctagaaagttccaaactttctcacattttcctgtgttcttccgagccctc |
| | caaactgttccatcctctgcctgttatccagttccaaagctgcttccacatttttgggtatcttttcagcagcgtcccactcct |
| | ggtatcaatttactgtattaatctgttttcatgctgctgataaagacatacctgagactgggcaatttaccaaagaaagacat |
| | ttaattggacttacagttccatgtggctggggaagcctcacaatcatggcagaaggcaaagaggagcaagtcacatctt |
| | acatggatggtgtcaggcaaagagagagtgagagccaagtgaaagggatttctccccataaaatcattagatctcatga |
| | gacttattcactaccatgagaacagtatggagaaaactgccactatgattcaactatttcccaccatgtccctcccccaac |
| | aatggaaattatgggagatacaactcaagatgagatctgggtggggacacagccaaaccatattagtcaggcatataaa |
| | aacctagatattagtaggtataaaataatggttttagttagttttgaacctttggagaggaaaagatcaaaccaataatactt |
| | aaatgatgaagttagtattctttccaataataattacataaatccaatgcaagctggctgaagtaaaaaagagaatgtatta |
| | gatgatataaattatgtctgtgagtagctttaggcatggatggatccagaagctcaagacatgttaccatgaccatgtctttc |
| | tccatatctgaatctaggttcctcccacatggtggcaagatgtcactgcagctccaaacttaaagccaatatttttagcagc |
| | ctcaagtgaaaaagcaccatttcctcaacagttgtaaaagacagggatatctttcaattgaccaagcaggatttcatgacc |
| | atctttaaattacgtatcgaatacactgattggctaggcctaggacagtgcctggaacactgggagacccgagcctcaa |
| | gtcctgctgcatccaggagttgaaattagtgtctcccagataacctggtctgagagttgaggattatttctcaaggctgaac |
| | agggtactgatttaaaaaaaaaaaaattaaatggttgtgatcagcctcttagtgaaaattaagtttttgtaaatattgccctc |
| | agatttcttgagacagagacaaaggggtgaaaattggggaataaatcatacagttatttcagcttgatttaatttattcatga |
| | agaccatcataaaatatgcaaagggaagtggagaagctgccccgtgtactataattaaacatccctactagcaagattaa |
| | ttatatttcctccatggtaagatttgcatcagggtgtggtcactagcgagctcttactggctacatttttgacctcagaggatc |
| | taaaggtagatttgtgtttaattgttttccattgggttgttaactgaaattaacttctaaagaagggtctatcaacagtatcagtt |
| | ctagatgcccgtaacaggacaaaacattatggggacacttctgactatgttgaggtgtgggtaaagtaggagaaaagag |
| | agcagaagatggaaaatggaggaaggagaaaaagcgagagtgaaatagaaaaggtgaaccttgtagaaagtgcca |
| | aaatgccaccagcagtcatcagaggggtgctttcttccacatgtccaatgacttatccttgagtaagtcaatgactatgac |
| | acaatgaatcaaattctgtttttcagaatgccagctcttaactctcttcatctcatttttgtttcttctcttgttattcatagtcctta |
| | ctgagcatccgtggctcccttttctctccaagacgcaacagtagggcgagccttttcagcttcagaggtcgagcaaagga |
| | cattggctctgagaatgactttgctgatgatgagcacagcacctttgaggacaatgacagccgaagagactctctgttcg |
| | tgccgcacagacatggagaacggcgccacagcaatgtcagccaggccagccgtgcctccagggtgctccccatcct |
| | gcccatgaatgggaagatgcatagcgctgtggactgcaatggtgtggtctccctggtcgggggcccttctaccctcaca |
| | tctgctgggcagctcctaccagaggtgaggccaattaaaattgcagctgatgtgaagagagttgtgactggtgcaggca |
| | ggagtgtttttccatttccacatctaagaatttgttgagtttgttgcccaaaggctgggagtttgttcaatcaagctgttaactg |
| | tcttgtgaaactgttctattcagacttttctacaaagtaattaaaaacctaggttggctgtcagagaatataattagaagtaat |
| | ctttcatcattattactatggtatgaaactcgccaaaaagcaaagcaacaatttatcaagcataatgtttgattaatatagtta |
| | aattaaatccaaggaaattaatgctcactaattaaataaatacttaaggattttgtgattgttgttcatttaaaaggagatttga |
| | atacttccacttgcagtagatactattactaaatagatttaaatcccatagtacaacattgcctctctttgcaggtcagagtgtt |
| | gtaacctttttagcatccactctaatgatctcaaccattgtaaatttatacatgaagagccattcaaaaagtacctggtttgga |
| | atcatgggctgtcatttttagagcagattccaatttttatattactgtcataaactcttattgtaaacaaagtggcccaaaacca |
| | atcacatggaaaaggatttcagctacatactagacacttacagggctatattattgaaatttacttcataaaccataagaag |
| | cttttaatgttggtattaaataaaattccattagctatcaagacatattttggcaatgtcacttgattgtattttatagcattcaaa |
| | atgtcttcttatgatttttttttcacatagctccatttattatcattgaacaagctctttgagccacattaaaatgatacggagttc |
| | gttttcagttacctaatggaggagcttcttatcttggattataaaatagccattatcttcttcacattttttgcatggcctctcccc |
| | acctccttctaccagagaagtgtccaggtatcctgcagtcaggttgaaccatgagaaaagtagaactttatagtggagga |
| | accaggaagaatgaagagaggacatcagctcttcttaaaaaatgatcatagataaccatgtaccagacactgtccagaa |
| | cactgtacatgtgttaagaatcatttaagcatcataacaaccctttttgatagttagtaacattatcccaattttacagaggag |
| | gaaactgaggcttgtgcgtggtggagctaagatttgaccccaggtatgctggttactgaacctacattctatcaacagtga |
| | aatattgcctcccactgagttatttttaatttctttaaatcaaaaagaagagatggttaaggaaataaacacataaacactttc |
| | attttaaccaatgtttctcaaaatatacttcactttcatactacttacatcagaatctcatgggaggcttcttaaaactagagatt |
| | cctgtgcctacccagaccttttcagaccaaaccactgaggagaacaaggtctgggaatcttcttccttaacaagcatccc |
| | acataattcttatacataataaagtatgtttatcactgatcttgataaatgttaattgggtaaacaaaagcaaatctacaattac |
| | catgcaggaatacagacagactgtcagtctgtcagaaattatttagcatttatcaataattatcataaatctcctgtcctatca |
| | gagatgatgggacaaatcgctgaaggcaaagttggggccagcttgaagcaaagctttgtgtggtccctttatttcctgctt |
| | cctcaacttcattctttaatcttacaatcttaagtgctttgaggcagggcactgtactattgcaaagttgagctgaaggtgca |
| | aacaaatgaagtaggctttggagaatgcagaggtgaaatgacaatagaaaataaatagctatgggcaaatgacaccct |
| | tgaaagcacatcatttcctgtactttacacataaattcaatcgagtaatgtcattagcagttttggaatctatttgaaaattaga |
| | caaatctaggtttgtacatgtgcttctgtgtagaacagaagggactagatgatcttcatgtgactattttttttttccttaaaact |
| | ttgcctctttctgacaagctgaaatattttaaattctaagaggcaccctttggattaaaagacttttatttttagagataggttatt |
| | tcttttcttactaattttacttgtttttttaccttaaaattaattttagaatgacctatatgaatagttatcaccattagtgacaatcat |
| | atgcaatgagtggaaattttggttttgaatatgtatgcattaaaataattcaacttacaaaagataaaatactgctaattgttca |
| | catcataataggatgtgaccaaaataaataatattttgatcatattattatattttgatcatattgttcttttagaaatagggaaa |
| | gtcctcaaaggaatgaaactttttaatttattattaacactcagacctgcatttgaaattctttagctttacctttttttttcctgtga |
| | taattgacatcattgtttgatctcctgaagtaggaataaatttccacccatgttgaaatcctgatgagtttattctggagtagg |
| | agattatcagatcattgtatcattactaaaaatcacagtcccccacattggtattatctccttaaattatagtctcagtgccaa |
| | gggggattgttttgtggattaagttgtttttcaaatataggacaaagttatagactagttctaaaatttagttttgtaattagga |
| | atgttgggaaatattacctgtgtctaatgaatgaaggcattttgcaactggaattcacattttaggggaactgttactgatgc |
| | atatgaaggagactttcaaacctttttgttcatatattaaactacctgaatatatgtctataaagatctaaaaactcaacctgg |
| | gtgaaaattaagaaacaatatgttttggtctgaagtcctaagtgggattggctgaaatgctaaaaggttatctgtccagtag |
| | tggacctggtccctccagcccaaatccctgggatagaggcataggaaagcccaccttgacaaacccagggctcccca |
| | aaagctgaaaatctgacagacttttaaacaacccccaaagaattatcattccaacaatatcttagtgagctttttacatctga |
| | gaaagcatggtgtatatttagttaaataacacctgttgtaggaatgctttgggctttgctgctttcaaaaatagtggttatttca |
| | tctgaaattctacttctagggcacaactactgaaacagaaataagaaagagacggtccagttcttatcatgtttccatggat |
| | ttattggaagatcctacatcaaggcaaagagcaatgagtatagccagtattttgaccaacaccatggaaggtatgttaaaa |
| | gtcctgcgtcacagttacttggtgctttggtaatgatgaaaaaacacttcataaatttcaataaaatacttcctgacttgatatt |
| | gtatcattattacacattttactaaataacagtaaaatccgtgcataactcatggattctattatcttccacagattttttttttttat |
| | atttagcctccagaaagctgctgcaaatgtaaggtatattttgaacaccactttcatacattaaattctaaacattgaaacttg |
| | tgtgcatgacgttgaaaagagtgtaatgataaatgcttatacttatgatgatgctaagccatttggattatattaactgcttga |
| | gacacaagttataaaatcctatgacttaaccagaaatataaattaaaaatgtgaattagggtttgatattaacttccttgaag |
| | caaagtgtttaaaattttgtagtcctacttttgcctttctctgaccagattcttacaatatatcagctttctctttagttgcagatttt |
| | atctgaatagttaacataatgtgtagcagtctggatctcagaatgccaaaataaagactttggggacagcttaatctgtgat |
| | caatttctggctctgccatatgttaaatgtgttaatttgtgactttgaatttcagtctcctcatcagtaaaatgtggatgatgatg |
| | tttaggcataaggttgttgaatggattaaataagccttcttagataaaacactgatgtatttggcatgcagaagacagttaat |
| | aaatattatcaatattagttgttttgttgttgttatttttgttaattcacatgtttttgcctttccatactgtaagtgaattcaaacaac |
| | tgtcaacttcaactacttggaaaatattttcatgtaaaatgtattctatcccccttccttgccctcctattccctcctctccctatc |
| | tctttacaaaccttctcccttgtaccccttcccaggtatgtgtgtgagtgtgagtgtgtgtagatgtgtcaagggagaagag |
| | aaaaggagaatgaaagcaaaagagagcaagcatacacgtccctttcttattgataattagattttctcttgagattggatag |
| | attcctggaataattcttttcctgtctgtatgcaaagatcccataatattattaataccaatacgaaaagcctgaaaatcacag |
| | ccagaaaaaattcacagtgtagacgactgtgtacatcacagacaagtcagtattacaaaacccaattttcatagtgtcctat |
| | ttcagtatcctaatgcaattcactgatttcaattgaatattaaactctagtacgttcttccccaacctcgcctgcgttagcttgc |
| | actccctcttccccccagctgccagtagcttgctcctccctgtccctccaggtaaatcttttgaagattgtctggccttccgc |
| | tccttgccatagcaaaaccactgagaggaagctgccagtggttctgctaccgatgtcagcagcatgtctgctccctaaag |
| | caggaagtagagaaggagacagggtaagtctaaatcaacagtcatgctttgcacttctgatagcattaagtttgagctaa |
| | ataagacattacttaaaaaacctcaaatatccacaagattggacttgccaactaattaagatttggagttcaaaataaatgc |
| | acccacacctttctccatggaactatgtgacatggggttgcttaggatggaaaggatgttctaggaataagtgcaatctag |
| | gaagctgaagactgagagtgttttcgttttattatctgcagagcttttgacttgtgtatttgtgagaaataatggccaagttttt |
| | attctgtttttaatcagttatctagaatgaaaactgacttttctttattcaattgtatgtagacacattgagtgtgacatttgtcaa |
| | ggttggttgttagcaatatcacatacatgcatactcaagcagacttaagatagtcctttttttttttttttttttttttggtttctgata |
| | atggtgcaaagttttcctggttgacataatctcttttcttggggatcctttcttctatgtctgattattgtttatttcacctttccttt |
| | ttatgaaccaggcttgttgatccggttggcaatttttgttctccttctttttaactacagccaagtctccgttgtccagggtaatg |
| | gatagcctcatgcttaatgaagcagtagtggataagcaaaaatgaaccatttgcgtttcaaatttttaaaagtgcaaaatca |
| | catagaaatgttttctggtcacacctttgtgaaggatggtgggagggtgagttagaagcgcctgaagaatcaaggtgag |
| | ccagcaaaagacacagatttactgtaagtgattctatgtggataacccatgcaggaataatggagatgtggctgcagttct |
| | ctcctgaatgctttgctttgttttaaagtgtgagattccccccttttttttggaatgaataattgaatgattttattttagaacttaa |
| | acaactttgctccggttattcctactgtcaagatgagccacacctgctgaatttcattttttaaacttttctccagtttattttttttc |
| | tatccagttcctgtttgcttctcgttatttgctaaatgacagctggcatggaaagaaaaaaccatattatggcagtatacaga |
| | caaaattaaattttgtagtttcttttttgttatttactgtaaataataatacctcctttttaccttcaatgtaaatataaggttatttgg |
| | gagtttagaagtatttacaaaataactagtgcataataactatatttttctctgaattataaatcaaatattatttattgtttacaaa |
| | gttatgtattaagggaaatggaaacaaactgggcacttgaagaatttcaatatccaaagagacaattgacaaatctattttt |
| | agtggaaattttaaaaacaataagcaataaattaatttacttaggaaaatagtattatagaattaattagtggcaaatgttgat |
| | tagtagaagaaacattatcatctgtggttgtgattgtccttttatatgctggttccacctttacaaggtttagttcatagcaaact |
| | gtgccagatatggacagatgttccagttgccacaatagtattaaagtgactgactagagtcaactatgccatggatttaaa |
| | aagaagaaaccttccctctatttcagtgctaagaggtggtggccacattttggcagaacaggtaatagggtgtacagcaa |
| | tgatattgacagaaaacaacaaattctgcatatttttccactcataagttgatgaagagattatcttgccaaaggaagatggt |
| | agacagtttttcacttcctaattccccaaatttctttgccaatattcaccaaatttcagtattttgggtgtgaccttaaagatctgt |
| | gcatttctgtcttctctcccaatgtttggttgaaattcttcttgacatcattgtacattttccttgaataaatgcattttaatataaaa |
| | ttttatgtcatgtttgatatgagagttatatatttaaatacatttaaataaatgtttaccatgaaaatgtatgaattatatgtatgttt |
| | cacctaaaatcctttgtatttttccagtaataaatgagttccactttgtgaaatgttgatttgtaacaacagtgaggactccagt |
| | tccttaggctggggtattttctcttcttttatgccctctagttaaatgagaaatgtagagagatggaactttgttgtgtctaatat |
| | gcaagcctataatctaataaaatttaatttgagacttttaaactgagattggtgacactgacaaaattatctaattagaagatc |
| | accaaaacatatctaatccaagaaactgacattcagtgtgactgattaaggttcttaggacatctcctgagatatctctgata |
| | acatatatacttcttgctctacctggaacatggatgagctttaagtgtatgcaatgcaagttctacccattagtttctagcagc |
| | cttgaagataagtatcagacagtttagtgttgccaatagaatcttggaagctatgtttagccaggatacatttggaaagctta |
| | ctagcctttctgtactgatcctttctatgacagcaaacccattgtaaaattttccctgttcctccagcagattaacccataatat |
| | cttttaacaactttagattttttaaattccttttaatttaaaccaaatctgcttaatagaaagtaagcagttttcatgaggattctaa |
| | ctttttttcttccagaacttgaagaatccagacagaaatgcccaccatgctggtataaatttgctaatatgtgtttgatttggg |
| | actgttgtaaaccatggttaaaggtgaaacaccttgtcaacctggttgtaatggacccatttgttgacctggccatcaccat |
| | ctgcattgtcttaaatacactcttcatggctatggagcactatcccatgacggagcagttcagcagtgtactgtctgttgga |
| | aacctggtaagcctcactgagagtttctcttcctcttgaaagagtttataattgccttagtgaattttacatattgctctcaaatt |
| | aaatatcaactaattggccatgtatatcttgacatcaaatgtttagcatcccttttaaataacaaaaaaatgttgctaccatagt |
| | gcaaaagagtcaaagaatttatgtacaatttgatttagaattgaatttaaattgcttatttattagaagatgattctgaattgtcc |
| | tccaaggacattgatctatagcaaaattctgacatatttttaagaaccttagaataggttctttaggacatgtctgtgtttacta |
| | aacaaatgataaaatatgcccaagtcagataattttgaaatatcacttgtaagtacttgaagatggactatgtagggaggg |
| | gacatcatctgggggattattattttttgtttttgtttcttctacagtcttagcaatattaaatttagaaattatttatttaattttgt |
| | taaaatatatattagcatttactggatacatattgatattataatatagtatactatagtgatagattttaaagtggtcttatacaga |
| | gagacaaaatgaagaaaatcacgcaaaaaagtatatttatacaagtataaaatgcttattaagttcccagcttgataaatga |
| | acatataagcaggttatatagagattgtaaataatacggtctaaagtataatatacaatatttttaggctctgaagtaaccact |
| | atattttcgaattatatttcaggtaggacttaactgaattaaatgataaagttggcatatgttggcctcttattttgtatcagatat |
| | tggactaaatggtttacattaaatctctctctctagattcccacaaaaccagtcttaagatatttactataaattgtctgtatttc |
| | gcagttgggaaactgtatccatgagaaattaacagagaaatggaaggtctgaatgctgtatcagactctgaaagctatttc |
| | agagactatcataagctatgggcaaagatcacagacgcttagagtaggaaaggataataattttacctagttcaaatttag |
| | agctatgtaagaatttcttcaacattatttctaaaaaaaagcagggtggtggggggcaattgaaacaagaaagcctttgg |
| | aggtaatatattgtgatccaaattgaatgagcttaagcaaaaataaagggaattcattacctcactttactgaagcccatttg |
| | gaactagttctaggttctaacctggctagattcaggttctcagccagcatcattgggaatcagcttcttgtctctcttcttccc |
| | aatatgtgaccttcctctctggctgggcattttcctactattgccaagattctatcaacttttattctactaatgtagctccatgt |
| | acagaaagtttgtgcctcctaactagtagctttaattaaggcccacaaattgaatatcattgacctggttggagttacatgg |
| | ccatttctaaaccagtcagttaccctagctcttggttttaatgccatattcgcctaaatcagaatcatatcttatcactggagg |
| | cagagtaaatgagtgaaattatgaagacaataattaggataccattaccagaaggaggatggattctagaaagaaataa |
| | acaataaatagacaactcaagctggggctggtcttacgtgttaaggaatgtaggatctgtatctaaggtgaattatggaat |
| | agtagaaaatccataggcaggatacacattcttctcaaatgcctataggatattccctaggataagttataggttaggtcat |
| | aaaacacacatcaataaacttaaaaaaattaaaataatacagaatatttttctgataaaaaataaaatgaaattagaaacca |
| | ataacaagtcaatgtggaaaatcacaaatatttggaaattaaacaacttgctcttaaataaccaatgagtcaataagatgtc |
| | atgagagaaattagaaaatactttaggatgactgaaaatcaaaacaaaccaaactgaaattaatgagggcagctaaaac |
| | aatatttaaagacaaatttatactctaaatgtctatagtaaaataggagatttcccaaattgctaatttaagctcctttttaataa |
| | actagataaagaatagcaaattaaacctgaagttaacagaaaagacaaatattaaagaagagaaaaacaacagagaaa |
| | aatcaataaaaccaaaagttatttctttgaaaaaatcaacacaattgacaaatctttagttgggctaaccaagaaaaaaaga |
| | ggaaagacactaattattagaagtaggaatgaagagaggatattaccatggatcttttagaaagaaaaagggagcataa |
| | gaaataaaataaaattaaacgccagcaaactagataacctatgtgaaattgaaaaattcctagggagtaacaagcgcct |
| | gaaactgactcaagaagtaatagactatctcaatatacttataataagtaaacattgaattcacaattaaaaaaaagaaaac |
| | ttcctgccaaaaaaagcctgagcccagatagtatcactggtgaattttgccaaatgttcaatggagcgttaacatcaatcct |
| | taacaaactgttccaacacatagaagagaagggaatacttctcacctcactttctgaagctagaattaccctgatattaatg |
| | ccagacaaaaataatgcaagaaaagaacacagacacaaatatagaccagccatatcccatatgaacataggcacaac |
| | aatcctcaacaaaatactagaagccaaatcacataacatatttagatttttagattatgtactctgaataagtgaaatttatcc |
| | cagtgatgcagggctggaccagcataaaaaattaatgtaatatatcatattaattttaaaaaactatacaatcatctctatag |
| | atgctgtaatcacatggaaaaagccaaagtgtttcatgataaaaacactcagcaaacttgaaatagaaaagaacttcagc |
| | ctgataaacaccatctcaaaagaccccacacctatcatcattcttaatagttactttagatgcttttatcttcaggtcaagaag |
| | aaggcaaggatatttgctcttgctacttcttttcaatattgtactgaaagttctaaccagggaaataagacaagaaaaagaa |
| | attaatggcatctagattggaacacaagaagtaaattctattaacgaatacataatcttgtatttagaaaatcctataaaatac |
| | acacacacacacacacagctgttagaactaataaataagattgcagaatacaagatcaatatacaaaactcaattatattt |
| | ctaaacactactaatgaacaatcagaaaataaaattaggaaaattctatttataacaacatgaaaaataataaaatacttag |
| | gaataaatttaataaaataagtgtgagatttgtacactcaaagctataaaatattgttgaaagaaattaaagaactactaaat |
| | aaatgaaagcacattttatattcatagattagaggaaaatattgttaagatggcaatactcaccaaattaatctacaaatttaa |
| | cgctattcatatcaaaatcccagctacctattttgcagaaagtgataaattgactgtaaattttatatgaaaatgcaagagac |
| | tatatgccacacaatcttaactagaaaaaaaataaagttggagaactcaaacttccaaattttaaaacttactacaaagcaa |
| | aagtaagcaagatagtttggtactggcataaggatagctatatacatcaatggaataaaattgaaattccaacagtaagtc |
| | ttcatatttatgttaaattaattttcaacaagaccactagacaattttattaaggaaagaagccttttcaacaaatggtgcttgg |
| | acaagtgaatatccacatatgaaagaatggaattgaacccttacttaataacatatataaaaattaagataggtcataggcc |
| | taaaggtagagctaaaactatgaactgttagaaggaaatttagaagtaaatcttcatgacctattattaagcaatgatttctta |
| | gatatgataccaaaagcacacacaatagcaataagaaaaaaaaggttcattggactttatcaaaattagaaactttcatac |
| | tgcaaacaatatcatcaagtaaaaagacaacttaaagaatggaagaagacatttgcaacccagatatctgatcatgatttg |
| | tatctaatatatgtaaaggattattataactccacaacaaataaaatagataactcactaaaaatgatcaaaatatttgaatag |
| | acatattgaaaaaggagttagacaaaaggccaataagcacataaaagatggtcagcgtcactggctaattttaggggaa |
| | aggcaaatcaaaactaaaatgagataccacttcacacacactaagatggctataatcagaaagaaagccaataccatttt |
| | ttatcaaggatgtggaaaaattagaatggttatgctttactttgagaatataaaatgatgcagtcactttggataataatttag |
| | ctgttccccaaaaagtttggggtagagttaccacatgacctggcagttttactaatttcttcggatatatatatctaagagaat |
| | taaaaacatatttcaacatgaatgcttatagaatattattcataatactaaaaattaaaaacaattcaaatgtctatcacttgat |
| | gaatggataaacaatatccatccaataaatgttattcatccataaaaaagaatgaagtattgctacaatctacaacatgaat |
| | aaaccttgaatatattatattaagtgaacaaagccagtcacaaaatttacatatcatattgctacatttatatgaaatgttcata |
| | acaggcaaatccatagagacagaaaggagatgagtggttgccaggcagtagggattagggtaaatgggagcaacaa |
| | ctaactaatgggtatgcagcttctttttaaggcgatgaaaatgttctaaaattcaataatggtgatgattttacaagtctgtga |
| | atatattacaagccactgaattgcatactaatatttcatagtatgcatgtatcactattttttcttccaatcacctattgatggatg |
| | ttcaagttgattccagatactttacccccatgttgcagtaaatgttcttgtatatatcacctttcataatgctgtattaacttctag |
| | aagatagatttccaggattgaagtattcccaatattaaacatattcatgtcacataaaaattagatgatatgcaacgaaatag |
| | ggaaatgtgactcatagttaatagattaaagcggtacatagaaatagatctggacaggaacaaattgttagaataaacaa |
| | aaaaggactttggaggcactattgtaaatatgttgagcaatttaaaggaaaaaaaggggtcataaggaataaacagaga |
| | acatgagcagaaaaaaatgaaaactgtaaaaaggaataactcagacattttataattgaaaagtacacattctaatatgaa |
| | aaattcactggatgtgtttaaaacaaaattgcagatgtcagaagaaatagttgttaacttggagcacattaatagaaattatc |
| | caatttgaacactagagaattaaaaggaaaaatgaactgagtctaactataatatgggataatatcaagtagtctaaatattt |
| | atataatgggagaatcagaaggagaaaatagaaagaatggaacagagcaaatatttgaagaaacaatggccaaaaatt |
| | tcccaaagttggtgaaaaatagtaacttacagaccttaatgttaccttataaaatgttagtgaacatcaacaggataaatata |
| | aagtaagccatacttaggcacatcttagtcatgctgctgaaaacaacaataacaaaaagctttggaaacaaccggaaga |
| | aaaatatttactatatacatgagactaatgtcttaccagaaataattcaggccagaagaaagtggccagataaaataaaga |
| | aataaaagcccattattctacagaaagtgaaactatccatcagaaattaaaatgaaataaagaattttaagataaacaaaa |
| | acctaaaaaagctgttaccaccagaactacactacaagaaatgttaatggaagttctttaggccgaataggaaaaatatc |
| | agatggaaatttgtttctgggaatgatggtcactagatatgaataaatgtgggtaaatacgaaagactatctttttttccttaat |
| | ttttttcacatttattttaggttcaggggtatgtagacagctttgttgcataaataaattgtaaatcacaggggtttggcataca |
| | gattatttcatcatccaggtaataagcatagtactctatagatagttcttcaattctcatcctccttccaccctccaccctcaag |
| | tgtctgttgttcccttctttgtgttcctgtgaactcaatgtttagttcccacttataagtgagaacatgcagtatttggttttctgtt |
| | gctgtggtaaattttttaaaaagacaactgattgtttaaagtggggatttataacataggtaagtgtaaaattatgaaaagatt |
| | tgaacttttttttttttttttgagacggagtctcgctctctcgcccaggctggagtgcagtggtgcgatctcggctcactgcaa |
| | gctccgcctccccggttcacgttgttctgctgccttagcctcccgagtagctgggactacatgcaccagccaccaagcct |
| | ggctaattttgttgcatttttagtagagacggggtttcatcgtgttagccaggatggtcttgatctcctgacctcatgatccgc |
| | ccgtctcggcctcccaaagtgctgggattacaggcatgagccaccacgccgggccagaacattttatctaaagtgttac |
| | aatattaattttacatagaaaattacaagaaagtatccctcatgaacacagaagcaaaaaatcattagcaaaatattatcaat |
| | aaaatctagcaatagagaaaaaagtaataaatacttcttaaacatgtggggattatctcagaaaagtaagattcatttaaca |
| | tttgaaaagtgatcaattaattggccatattacctctaaaagaataaaggaaagcctaagatcatctcaatagatgcagaa |
| | aaggatctgacaaaactcaacagtcatttgtgagaaaaactgtcagtaaactaggaatagaaagtagctatctcaagttgt |
| | taaggacgtttcagaaaaccctacatctagccgggcgtggaggctcacgcctgtaatcccagcactttgtgaggctgag |
| | gcagtggatcacttgaggtcaggagttcgagaccagcctggccaacatagtgaaaccccgtctgtactaaaaatagaa |
| | aaaattagctgagcgtggtggcaggcgcctgcaataccagctaacagggaggctgaggcaggagaattgcttgaacc |
| | cgggaggcggaggttgcagtgagccgagattaagccactgcactccacacttcagcctgggcaacaagagtgaaact |
| | ctgtctcaaaaaaaaaaaaagaaaaagaaagaaaaccctacacctaatattttacttatcagtgaaatgttgaggtagtga |
| | attcttgccccttagaaaaattgagtgctttccccttaacatagacaaatatgtctatttttaccatttttatttgaccttgcactg |
| | gaagttttatcaactgaactaaaggaaaaaaaaacaaaacaaataaaatgcataaatattgaaatgtggtaaattatctattt |
| | catcaacatgatcatattgtagacaatcctaataaatctttaaaactgattagaaataaacgcgatattaaatattttatttacc |
| | ataatatcaaaaaacacgaagaatatagtaataaatttaacaaatcatttcgagacacctattactaaaaacctcaaaacat |
| | ggctgagaataattacagaagatttaaacaaatggaaatatatatgccatgttcatgcaatggaagattcaatattattaag |
| | aaattaattctacccaaatgtatctatcaattcaacccaatcccagtaaaaatgtcacagattttctttgtagaaattgatgaa |
| | ctgactttctatgcaaatacacagtgcttaaaattgccaaaacaatactgataaaagaataatgaaaacattaacctacctg |
| | acttcaaggcttgttataaagctgtactaatcaagaatctacagtattggcataaacaaagatatcaatggaacataatgct |
| | gagttcataaatagatccaaactacatgtcaatggattttttgaaataaacactacagaaaggaaatagaagaaaggaag |
| | tgttttcaacaaaatatcaggaacacataaataaatgtatggaaaataaatgaacctccacttcgatcttatgccaagtgcc |
| | agaatcaataaggattgtagactaagcagaaaagctaaatcaataaaacatctgaaaaaaacacaggagaacatctttg |
| | agaccttggggtaggcaaaaatttattggaaaacagacaaaaagtgctatctattatttaaatgtccatgaaatttcaaaca |
| | atggagacctactgaacaaaaaagaaaagtctagtaatatacacatgggtgaatttcaatttgaagtattcaagtaatattt |
| | ggaaaagtcactaagactgtcaggtctctgaggtaaccacctaggtagagagagattccatttactgagttagcaaatat |
| | cagaagagaagcaaaactgtggagaaaaagcagtaagatgaattttggacctgttgaatttgagatgcctgggagatag |
| | tctttcaaatgtaggtattgaataggcaggtgggtatgtatttctagagactaggaggtatgcttgaacagaaaaatagattt |
| | tgaaatatgaactattataaaaatgtaacttattataaaaggaaaactaaagtaagaggggtctagacagagagagttctg |
| | aataaatccagtatcaaatgatttgttagaggaagaaaatcaggtgagtagcatccagggaggtgtggatcacagaagc |
| | taagggcagaaaatatttcaatgaacaaggaacagaaaacaatgcctgaaacttctaaaagggcaagcaagcaagata |
| | attgtttaaaaaatttcgtttggatttattggtgatgtcggtcttgttggtgaagtttgttaaagccatttggtgggttgtggagt |
| | aaggtgaagaaatggaactggcaagtgtagacaagtatgcaatcttcgaaagaaatctggccataaggaaaggataga |
| | tggtggcacctggaaggggaaatagagtgcaaggagagtttctcttatgacagtggtgatatatatattttttgtttgtttgtt |
| | tgttttttgagacggagtcttgctctttcgcccaggctggagtgcagtggcgctatctcggctcactgcaagctccgactc |
| | ccgggttcatgccattctcctgcctcagcctcccgagtagctaagactacaggcgcccgccaccgcgcccggctaattt |
| | tttgtatttttagtagagaagaggtttcaccgtgttagccaggatagtctcgatctcctgacctcgtgatctgcccacctcgg |
| | cctcccaaagtgctgggattacaggcgtgagccaccgcgcccggcccacagtggtgatatttttaagggagagaagg |
| | actcggtaactgttactttctataaagaaacgggaagtagccagtagtagaaatgttggttaattgagaatgaattccttgc |
| | aaagtccaaaggaaaacacataacacaatttgagagattagctcaaaacaggggcacttctttcattttaacaagaagag |
| | aaaggcaaagcacagttacggatgtaagtagtctagtagataaagggaaacaaagttcaggctggctgactttcatcac |
| | ctctaagacgctgaagtactgagactgtgcttctcagatgggaaggcataagcaaaaatggctgaggttatgtgcagaa |
| | gagaaagtttgaaatagttttagataatagaaatggagaaaggaaaatactgcttccctttcgccaacaaaaggaaattttt |
| | ttaagagttcttactatctgtagagctagccgtgagcatgtttattacagcttacatgacatcttagcaccagtctcatctgca |
| | gggccaagggaggggaccaatccatttggcttggtgatggaagcccgcactgctaggtaatcatttggtaagtttttgga |
| | gggctagaaagatcagagacagagccaaacagttgatcacaatgagtcagttgcacctttcatatgaaaataatattaatt |
| | ttattgacttaatccgtgtactctttatcatttgataaacattatatatagtgaacaattattgatttgaatgcaaagcatttgtag |
| | atactaagttgttggacctaaaccaattttttaaaatcagaatttaatttatatttgttgggagtaaattaagttgctcaataatta |
| | ttcgtgtttcaagagtatttgctcatataatgaactacacttctcatttaggtcttcacagggatcttcacagcagaaatgtttc |
| | tcaagataattgccatggatccatattattactttcaagaaggctggaatattttttgatggttttattgtgagccttagtttaatg |
| | gaacttggtttggcaaatgtggaaggattgtcagttctccgatcattccggctggtaaattaactgggagtgttcataaaat |
| | gtactttgtaattaattagtcttcattctcatctagtaaaaatggcaagatttcccatcattataatattatttgaatacacttctaa |
| | aacaaattggattgccataccaccaaatggtagtttcttcttcatcatagctttaataaagttcacttaaatgaatagtctaca |
| | cttctcttcttagttattgaatggaaggctaatagagaggaggaaacagggagtcacagataaactcgaatcacaattaa |
| | acaacaccatagtcaactctcagttatctgaggtttgcataactgcgtacaaagcttccttgggacctaggatgagctccc |
| | ctttctgccaggaactaaagaattatggaattgttcattgctcaccttgtccccgtagaggaaagagttaagacaggggat |
| | agtgtacaaaggagaaggataagcaaacagagctccccatatgactgctgccacatcagaaaatcaccaaatcactctt |
| | tgaaagagttaactgtactatattttgttaattttaaagaaagtatctttctttgatcttttataaaaactattagatcttaaaattca |
| | gagataaaatatcacttgacacatttccagtgaaagtttgatatgttttgttatactattactttgagttggctctaagttagtga |
| | tttattttcaaataacagaggctgtacacggttactaaggacacgttcctatagatgatttaccttagtagtgattaggctgaa |
| | gactttttcatgaaatctgtttacaatttccctttctgctttcaatgttcaaatttgagttgtaatccttagaactatatttccttccc |
| | taatcctcaaagatagttatgaatctaatttgaatctagaaggatgcaaaaaacagaacaaaaatttaaaatgataaaaca |
| | agtaatatgggcaagaacttaaaaaaatatatttagtaaaccttcatgatagtgtgatgcagttaagggaaataggaagca |
| | tagtatcactagaatcttacttagtgtgtcaggctcttttgcataaattattctctggaataaattaaatactttggtgcatgtatt |
| | tactcctttgggtcactttgatgccattaaataatgcactactttcagcctgacatttactgaagcatcagaaataaaatgctg |
| | ctgctctttaaccataaatggtacttcagtgaactctaaagctaatacaaccaatatgtcaaacacaatgagaaagacattt |
| | acacactacactgaattaagtctatgaagatataaaggttaaaaagaagcctagcgttttacttaagtttaagtatttttgtatt |
| | tgaatataatatatgtttaaaatatagcctaaagttacagcaagctaaagatatagctagattaaaacaatctaaagacaaa |
| | gaaattagttcatttctgcttccactttatgtaatttaagtgttgatattattctcacctgtgcatttcagcatatttaaagtacact |
| | gaaaactatatctgctttggccttttaaaaataatgagagttcctacttctctgaaactggatctctgctaattaaccaccatta |
| | atctgaaatatcttaattccttaaggagaaacaaaagtgtatattacatatgcttatgtaggatacttgaaaatttggtgtatct |
| | tattaaactgccaatttaaaaactgtataatttaattatttcatttacagtatggaccatttcaaaatgaaaaaaagaatgctct |
| | atggtagcaagtcactgctatatttgttagtgatcatttgacaaataaataattcatcattctataattgagacagttacctgta |
| | catttgccctgttaataaaattacagatttttcccttcctgtgtccatgtgactaacctgcacattgtgcacatgtaccctaaaa |
| | cttaaagtataataataataaaataaaataaaaataaaaaataaaaaaaaaaaataaaataaaattgcagatttttttagaa |
| | atgcagagcattaacactgttcttgcttttatttccagctccgagttttcaagttggcaaaatcttggccaactctaaatatgct |
| | aattaagatcattggcaattctgtgggggctctaggaaacctcaccttggtattggccatcatcgtcttcatttttgctgtggt |
| | cggcatgcagctctttggtaagagctacaaagaatgtgtctgcaagatttccaatgattgtgaactcccacgctggcacat |
| | gcatgactttttccactccttcctgatcgtgttccgcgtgctgtgtggagagtggatagagaccatgtgggactgtatgga |
| | ggtcgctggccaaaccatgtgccttactgtcttcatgatggtcatggtgattggaaatctagtggtatgtagcaaaaacatt |
| | ttcctcattttcattaaaagataatgtaatcattaaaaagtgtgttcaactgaagaatattttgtattttttaaatcaaggccactt |
| | cctattgtctattactcatgactgtaagagccatgtatagtttagaccattgtaatccacacaaacccttaaactaccttttga |
| | accaaagttattctttctttcattatccttcttgctacaaggagagaaacttttctgttatttatctttcagttcttgtactagagca |
| | tggaagtgttacttagaacactcattttatttataagtactagcaataacacctgaaaacgtttcagatttggttttctacaaatt |
| | taaaaactagcaacaatctcagtttattaagagctcatggggttttcggtgcctagaaactatggtatgagcaagtaacatt |
| | gtctctaaaaacattaattgtcatttctgcataaaattaaccacccctaacaccatatatatttaggatagttagctcttcttgtt |
| | gcattgatcccttttaccattatgtagtgtctttctttgtctttttttaatctttgttggtttaaagtctgttttatcaaagactagga |
| | ttgcaaaccctgctttttttttttctttccatttgcttggtaaatattcctccatccgttttttttgtgcctatgtgtgtctttgcatg |
| | tgagatgggtcacagcacaccgatgggtcttgactctatccaatttgccagtctgtgtcttttaattggggcatttagcccattta |
| | catttaaggttaatattgttatgtgtgaatttgaccctgtcattatgatgctagctggctattttgctcattagctgctgcggtttt |
| | ttcataatgttgatggtcttaacaatttggtatgtttttgcagtggctggtactggtttttccttgccatatttagtgcttccttcag |
| | gagctcttgtaagtcaggcctggtggtggcaaaatctcttggcatttgcttgtctgtaaatgattttatttctcctttgcttatga |
| | agcttagtttggctggatatgaaattctgggttgaaaattcttttctttaataatgtttaatattggctcccactctcttctggctt |
| | gtagggtttctgccgagagatctgctgttagtctggtgggcttccctttgtgggtaacccgacctttctctctggctgccatt |
| | aacattttttccttcatttctaccttggtgtatctgacaattatgtgtcttggggttgcttttctcaaggagcttctttgtggtgttct |
| | ctgtatttcctgaatttgaatgttggcctgtcttgctaggttggggaagttctcctggttatcctgaagagtgttttccaacttg |
| | gttccattctcccagtcactttcaggtacaccaatcaaacttagggttggtcttttcacatagtcccatgtttcttggagacttt |
| | gttcgttccttttcattcttttttctctaatcttatcttcatgctttacaaatttaactcaacatggattaaagacttaaatgtaagac |
| | ctaaaaccataaaaaccttagaagaaaacctaggcaataccattcaggacattggcatgggcaaagacttcatgactga |
| | aacaccaaaagcaatggtaacaaaagccaaaattgacaaatgggatctaattaaactaaagagcttctgcacagcaaaa |
| | gaaactatcatcagagtgaacaggcaacctacagaatgggagaaaatttttgcaatctatccatctaatatccagaatcta |
| | caaaaaacttaaacaaatttacaagaaaaacacaaccctatcaaaaagtgggtgaaggatatgaacagacacttctcaa |
| | aagaagacatttatgtggccaacaaacatatgaataaaagctcatcatcactggtcattagagaaatgcaaatcaaaacc |
| | acaatgagataccacttcacgccagttagaatggcgatcattaaaaagtcaggaaaccacagatgctggagaggatgt |
| | ggagaaataggaatgcttttacattgttggtgggagtgtaaactagttcaaccattgtggaaaacagtgtggcaattcctc |
| | aaggatctagaaccagaaataccatttgacccaggaatcccataactgggtatatacccaaagggttataaatcattctgc |
| | tataaagatgcatgcacacgtatgcttattgcagcactattcataatagcaaagatttggaaccaacccaaatgcccatca |
| | atgatagactggataaagaaaatgtggcacatatacaccatgaaatactatgcagccataaaaaagagtgagttcatgtc |
| | ctttgcagggacatggatgaagctggaaaccattattctcggcaaactaacacaggaacagaaaaccaaacactatatg |
| | ttctcactcataagtgggagttgaacaatgagaacacatggacacagggaggggaacatcacacactgaggcctgtcg |
| | aggggtggggggctaggggagggagagcattaggagaaataactaatgtagattacgggttaatggatgcagcaaac |
| | caccatggcaagtgtatatgtatgtaacaaatctgcatgttctacacatgtatcccaaaacttagagtataataataatttaaa |
| | aaaattaaccatacccaacactagtgtcctgaatcttgaaggcatggagaagttgggaaggcatgggaagataaatata |
| | acaaagtgatataacatgtactcaaatagaattaaaaataggaagtaactaatatgtgtccaaaaatatgaaaacaaagtg |
| | ccatgtgtcaagtttacaaaatgtaaaccttgctttacaataggaaggttgatcagggaagtctttgtcaaagagtttggac |
| | ctaaaatatatttaactgagatgtaagatttagcttggtaggaagaaagaccatcccaaacaaggaaacaaggtacccag |
| | tgactgagggatacaggacagtagactctgtgagaagtatcaggctcttatgctttaaatatgaagtaattacaccgagtt |
| | gcttaattagaacccaaaccaatggaatagaaaaatgactaccataacaagtaatttaatgtatatactcttgccaggctca |
| | gtggctcacgcctgtaatcgcagcattttgggagactgaagtgggcgtttcacttgaggacagtagttcgcgaccagcct |
| | agtcaacatggcaaaaccccatctctactagaaatacaaaaattagccaggcgtgatgatgcacacctgtaatcccagct |
| | acttgggaagctgaggcacgagaattgcttgagcctgggaggcagaggtggcagtgacccgagattgtgccattgcat |
| | tccagcctgggtgaaagagcgagactctgttgcaaaaaaaaaaaaaaaaaagcatatactctttagacatgatttcctctc |
| | atataaaggtaacctccaagtccccaaagatagagaaaggggaagggaaaaaggcaaagtattattttatttttattcatt |
| | gccaaatttcagcctcttcaacattacttttgataattctgatctatttttaaagtaacaagaaacataaacagtgtacaatcta |
| | gaattataaacagtggcttaaaacaataaacactgattacttcatagtttctgtgggtcaggatttggggaataagttagctg |
| | ggtggttctggtttaggatcagtcatgaagttgctgtcgagatgttagctgaggttacagttatcttgactggggctggagg |
| | atcagcttctaagaaggctcaatctcatgattattggaaggaggtttcagttcctttttggcagttagttgaaggtctcagtttt |
| | tctctgcaggaccttttccataggactgttgagtgtccttatgatatggcagctggcttcttccagggaaggtgatgtaaga |
| | gagaaggcaaggagaaaatcctctttatgttctactcttgaaagtcactcttcaccacttctgccatattgtattcattagaag |
| | ctagtcactaagaagagctcaagctactataatccccaagacaactttaaaatgtttgctttcagaaaagtataagatcaca |
| | tagaacagaaagtgccatagggttacatagaacagaaacaaagaaaagataatataattatgttatagatttgatttcatttt |
| | ctctgtatgtatatttggtatatgttggaagaagaaaagaaaacgcagagaacagaatcctttatgacaacatgaatgatc |
| | agacagcaatggggaattaagaaatataagtttgggaccagattgggtagaatttaatttatgaaaaggctgactgtgcat |
| | aataaaatgtattttcctttaggcaattcaaagctacagatgattttttatcaggaaagtgacagtgaaccagtgatatttttca |
| | gaaatatacgtagcaggagaatgcagaatagatttaaagaggatgaaactcagcccaccacatgttatctattagtttact |
| | gaaattaacatatctctctaatgtataaatgtgcagaaaattgaagttgaaaagagaatttcaggaaatatcaagtacttatg |
| | gttgacatcagtattaatttagattgtgatgtatgcataaaaagatatagtttataaaataatcatttccatctactgggtgtaa |
| | atttaatttttgttcttttaagagagaaaaattaaaggttctcctttctttttgactatcagttaaaataacttctttgtcttgtgata |
| | acctgggtatgtttctggagtagctaaggtagtcatatatatcatgtttaccactattaaggaaatgtgcttatataacatttgct |
| | taagactgaatgaacttgatatactcactccttactacaattcttccttcctattctcactggaaaaatgggaaaggtgtccc |
| | aaagacaaaatggcataacttccttttaacacacatgaactatcagatgtggctccacccaaatagatgtagtagtcacaa |
| | tggatgggactgccagcctagtctacagacaagacagagctgggaccacaaactactgtttcccagaccaggatttttat |
| | gagccattcttagtttccagacacgatggcaagagacccttcattggttgaagataggtgctgcagaaaaagaatgtgac |
| | tttctgaaaactgatagttctagaagcagagaagacaacttcctctctccctaagtgaaggtgaggcaatagcacacagg |
| | agggatgtgaaggttttggcttcctctcacaagttgggaatcaggatggagaaacaattaaaatatgtaatatgtttcaacc |
| | ttgaattcaaaatggaaattatggtaacatttccattccaagaggctaatttgagacacaagaaagagttgatttcatttact |
| | gagctagcacatttgtgaaacaggattcaggatttcagtccctgagtgagcttgctgaactgttttctttctttttttttttttttttt |
| | ttttttttgagacggagtctcgctctgtcgcccaggctggagtgcagtggcgggatctcggctcactgcaagctccgcct |
| | cccgggttcacgccattctcctgcctcagcctcccaagtagctgagactacaggcgcccgccactacacccggccaat |
| | tttttgtatttttagtagagacggggtttcaccgttttagccgggatggtctcgatctcctgacctcgtgatccgcccgaggc |
| | ctcccaaagtgctgggattacaggcgtgagccaccgcgcccggcctgaactgttttcttaaattgtcatggatcacacca |
| | aacacctgtgccagctgttatgcgcatacccttcggtaacaaaggaagtccagaaaaagagaataacttgactcacaca |
| | aatatttctagggaaataaggtaaataaaaagatagtgttgtggaggataagttggataatagtaagtgataacagctaaa |
| | ctttctcaaaggttcactatgtgccaagaactgtgctgaaagccacatgaattctgtcactgaatcctttcaacaaccttgta |
| | agctgggcactagaaaaacgattacatttattttatagatgaggaaactgaggctcagattggttatgctacttagtaggta |
| | acagaaaatcgattcttacctagcactcgaattctaaaatatgtgctcctctatgtcaagtaatctatagaactaagataaac |
| | atgctgatgaaagttagtgtctagtgggtattaataaacgcggtttcaaaactgtgtcaccacgggtagattggctgcttta |
| | aaaaaaataaaaacttcaatggatttatgagaaagaaaagtcatatgttccagggatatttatttattttcctgacagtggaat |
| | agcattgaattgagttatcaattcataaaagatcagagaaacaattcgaaaattaatagtaaaccctattatactgactaaat |
| | atggtagcagttcaaagaaaggaagtatcggtaagagtaaacatggaatactgtttcctcacttattctgcaaacatcaca |
| | attaggagaaaagaccttgtagttagactttcaaaaaaaaaaaagcttctctacttattaactgtggtcttggacaagtcatg |
| | taagttgtgcagaagcattttcatctgtaaaatagtaataattcctgccttatagagttgtgagaaataaatcacataaacctt |
| | gaaaatgctttgcacaataactggtatttactaagagctcaactaaaaagctggttttacttttattgttattatgatctggtatt |
| | gatactgctctaggacttggctctaaggcatgtttctgaccaaaagatctcccaatctatcagtaaagctgtgcttgtttttttt |
| | ttttttttaaagaatccaatataatgtgatagggatgtggatgagaaatttaacaggacagaatgagaaatgggagcaggc |
| | tatataaatgtcacagtaaatgacatttgtaaatagagtatttgtccagtacagaggcaatataattggctcccgccctggg |
| | gaaggattgatggatgtgtatcaagaaaaatttccaaatagacaaatgacagaactttaatatactttaggaaaataagtct |
| | aggaaatagcaccaaaatagataaaataaaaattttacatgcaattttttcttcctttgtctgtttttttaatccaaataataagtt |
| | caaaagcaaattacaataaaacataattttattggtaaattccagaggcaaaggagcaggtctggtcttaatgtgattatca |
| | ggagtcatagtatagagactgacagattgtcagtacactctcaaaatcaaacgtggtcttcattggatcttacatatttttact |
| | ttaaaaaaaatcaccattggttagactaacttacaactaattagacaaaggtgctgtaagcctcattagcatgatagaagc |
| | atgagaatatagcaagaatgtagaatcctttttattgaagttttacttaaaaattttcctaagattctacttttgtactacagtttg |
| | agcatccctaatctgaaaatttgaaatccaaaatgctcggaagttcaaagcttttgagcaccaacctgatactacaagagg |
| | aaaattcacatctggcctcctgtaatgaatcgcagttaaaccacagtcaaaatgttgtttcctgaactaaattattaaaaatat |
| | tgtataatattaccttcaggctatgtgtatgtgtatgaaacttaaatgaattttatgtttacacatgtgtctcattctcaagacttg |
| | tcattatgtacatgcaaatattttaaaatctaaaatccaaaacacttctggccccaagcatttcaaataagggatatgcaact |
| | tgtatttactttgtgcatttgcccccctttactgctatatctttcttttgttctgtatgttatgtgtgcttaaataatcaggaattcatt |
| | gatattgtcaatcaaatcctgaaaaaaaattatatgactcagtcttgtacccctgagaatgtctgatttcttcgtaagttgtcttt |
| | ttttttttttccacaatagtgagtttaatgtcatgaatcttttcactcattcatactggtggagcctatttttaaagacccaatttgc |
| | agactgattactgtccttattcatggcaatacttcaactccacaatctttaattcaacaataacatcataattattgtataataac |
| | cattttatagtatttctcactattgtataattatagtagccataattgtcttaataaaaattgggacttttcatccagcaataaata |
| | cgtttttgtctgatttgtccagttatctaggtacaaaaaatggtacaaaggcacaaaaataaaatcatatttaaatatattggg |
| | ataattgttgattttaggaataaattatcagtgtttccggaaatccaaattacatagtcaaaatagcatctgtattaggccattc |
| | ttgcattgttacagataaatacctgagactgggtaatttataaagaaaagagatttaattagctcatggttctgcagtgagct |
| | tggtgctggcatctgcttggcttctggtgaagtctcaaagagctttcaatcatggcagaaggcaaagtggagcaagcatt |
| | tcacatgatgaaagcaggaacaagcaagagagaatgtgggcaggaggcactacaaacttttaaataacaacatctcat |
| | gagaactcactaacatgaggacagcaccaaggcatgagggatctgaccccatcatccaaacacctaccaccaggccc |
| | cacgtctagtgctggggattacaattccacatgagacttgggagggggcaaatgtccaaaatatatcagcatcccaaata |
| | aaagggttttttttgtacagttgtctatatttatcttttggaactgagcttaatagaaatgtttcatttagcaatgatttcagtatttt |
| | ctgcaatgactaaaaagcaaatagtgataatagtattattttatattgaccaagcatttttatttcattcactttttttcagaatag |
| | tgtatcatgaattagcagaaatgcatgttagaataaaataaggtgtcaagaacaatcttagaaaactaatgatggaaagca |
| | attgaagcaatagaatgttttgatcacctgtttttcctgctgtgtttcaggttctgaacctcttcttggccttgcttttgagttcctt |
| | cagttctgacaatcttgctgccactgatgatgataacgaaatgaataatctccagattgctgtgggaaggatgcagaaag |
| | gaatcgattttgttaaaagaaaaatacgtgaatttattcagaaagcctttgttaggaagcagaaagctttagatgaaattaaa |
| | ccgcttgaagatctaaataataaaaaagacagctgtatttccaaccataccaccatagaaataggcaaagacctcaattat |
| | ctcaaagacggaaatggaactactagtggcataggcagcagtgtagaaaaatatgtcgtggatgaaagtgattacatgt |
| | catttataaacaaccctagcctcactgtgacagtaccaattgctgttggagaatctgactttgaaaatttaaatactgaagaa |
| | ttcagcagcgagtcagatatggaggaaagcaaagaggtaaaaatgtttaaataaggagatattttggtgttatataattctg |
| | ttgtttaaaattatcaggtgtttttaaattgcgtgtttccttcctgttaagaaaatagaaaatatctgtctagcaatatattttccat |
| | ggaaaagttggtaataaataaattaatgatagattaaaatatagctagattaacaatatgctgacttatgtttccaatactgac |
| | attttgaattcttgacagtattcttgatatgaattttttcagtatttataaataattttaaatttctcaaaatgcctcaatttctccact |
| | ttcttccttgtaatttgcccacaacagtgttttttgtacgtactggaaaaatatctgatgagagggtagttgcaattctcatctt |
| | gctatgttcttagttcttaattcttacgaaatacgtcataaaatagtattgtattttgtttgcacagacatatttactcaaggaag |
| | atctgattgggatcttggcttgatatttatgtatagtttatctttcctgaagtcagtcagtttttttgaagagaaggtattgatga |
| | ggaatcacactaaaaacatatttaaccctactgagctcagtgttcactgtttaaagaaacaaaaatccttaatacattataga |
| | atgtaaaattctgaatttaccaactcagtaagtcctggtaacttaatgtattctttgatttacaagaagggtatgagcaacag |
| | aatatattttttgttttgtttgctattaacctgttgctcaataagtacagagttggaggtaaagagaggaatttaaaaccttgat |
| | atttaattgtttatacaaaaatgaagacaagatttccagtaattaaagtttgcactaactaacaaaaataacaaggaaaaac |
| | aaagattcgttccttcctcatacgaactgtttggcgaggaagataaaagcttctattcctgatgtcgggaaagaaagaatg |
| | acgacatgggggagtgtgggcactgaaaggtaaaatttaagtagcacaacatgatcatgataattaacaatcagccaaa |
| | attatgagggaaaatatagttataaaaaaagaacaaagatgggtggatcacgaggtcaggagttcgagaccagcgtgg |
| | ccaacatggtgaaaccctgtctctactagagattcaaaaaaattagccaggcgtggtggtgcgtgcctgtaatcccagct |
| | actcgggaggctgaggcaagagaatcgcttgaacccaggaggcagagattgcagtgagccgagatcaccccattgc |
| | actccagcctgggcaacaggatgaaactctgtctcaaaaaaaaaaaaaaagaactagctatttcagacactttttctgtatt |
| | tatttgataaaattactaaagagtatgttattttccattttttcttgtttgtaagttacgtagtattgctgttagtgattaggtagaa |
| | gtagatgtttaatgggaaattcagacaatctttgaatataggaaggtataaataacagggacataggtatcagtttcacaa |
| | gaaataactgatgagattcaagggaaaagtaataaaaccttctgtcctggggcaaagaattactttaattggttgaacttaa |
| | atttttactaactagattattgtttgaaagttgaataatatcttaaaatcttattaacaaaattttgaacaagtgttgttacaatagt |
| | tgggttatgctggaagggtggagtggcccaatttcatatacagtgtactgctcttatagaagctgaagtcggcatttataaa |
| | atagaattcggtcatttgaattttgatgtatattcccctctcattattttgaaattatgcctaatggtgaatatttccctaatagtaa |
| | aaaaagtcaatttttattttcacacatgtttagttttaggctgtcatataaactaagaatgaattatacagtatcaaacgttgaa |
| | gccattggctagtttaatcttttagctaagtttcagtatcttttgaggaatgtttaacttgacatccagtcttcttaactttaagag |
| | attttacagccgtgggttttccaaaagagcgtgtattttgccttaacttaagccattatgtctgaagtaagagggaagtcca |
| | gtgatgtggggtttagagtagggacatctcttgtttctcttgttatcattaagctttttgatttgttttcccattaagttagctctga |
| | gttaaatactctaaaataatatttgtgaattcagtatttcagaattggaggaagagaactgacctgccaggtggaagcaga |
| | caggattattttattgcttgagttgtggagtccttccaataccttcccagcatagagactgttacttcagtgttaacattatttg |
| | gaggggtttttaattctggctttatatcaaactttctagacataaatttataaaataataaatgatgagggttatcgccgtgaa |
| | agaggttatgtgtaggttttgatctttcagaattttacctggtagctctacactaaaaaactagagaattaaaacaattattga |
| | agaatttcagacactcgcatttgaaatagcatttcttgcctgccttctagtcatttttgtctggtcatttttctaactgggggaca |
| | ggattacattgttaaatatcacaaagtagtaagaaacatcatgaggcttattaccaatcctttctaaattaattttttaattaaag |
| | aaaaaatgaggcttttttactggaatgtctaaatgaatttttttataaggcagactgagtggactcagaggttttttaggtgttc |
| | acagtaagtcctctgcaatgtctttgctaaatttgtatgattcttcagtagttttctgtagattctctagagtaggccatttaaaa |
| | tcatgtcataatcccctatgctttaattttaatgttatttcgattatattaatgtaattccttttgtgattttgaatgattgttttttc |
| | ttttagagtatttaataatgtggaagccatgcttgaatgactatttttcgaagtgaaatttagtagtgcgatatggtgaccttcacc |
| | gcttaccattcttacttctcacaggagtaaaatcaagctggagccatcaagaatgcagctctggtgttttttaaccagccag |
| | aggctcgtgccaccacttttacccaggttacccaagcaagttgtacatctataaatataatcagtttctaaatgacttttgact |
| | ggcctgcatgttactcagctacgttccttgcccttccattggcagtaaaataaaaacatgcacagctgctattatgctgagt |
| | catacaaagcatggtcaggcaagtctgacaaccctaacttaaaaaaaagtgatttagctgctaattttcttacatagatttta |
| | atagaaattttattcaatgaaaagtaaaagtgcatgcctttatggattatttaatttccttttaatgttacagagttttgaacatatt |
| | aggagcccaaaggagaaatgtaggtgctctttgaaaacttgcaaaaatgctttttatcctctgtctttaaaaaaaagatagc |
| | ccagttactgtacttaagtcttgacagttttttatttagtgtaatgtttttctgaagggtaatcttcaaattaaagcaatcccttatt |
| | catatgcaaacttcccaaaggatgttttaatgtgataataatgtaaatgaataggaatgtctgtgtttcagttgctagcagcat |
| | gggtataatatttatctgcttcattttagggaaaatggcactgctttatttaggagttgaccaacagtattttgtatttagaatat |
| | aatttctttggaaagtctgtttatatttacccttaaaactcttagactgaaagaaaaggaaatcatgtcttttgtataccaaata |
| | ataataataatagtgataatgagagacttatagatggtatgctccttctaaaaatagatttagagtccatcttcttcattttcttg |
| | gctcctctgtgctttctctcccctctattttattgagacctgctggaaaactttctcccgaagaatattatttaaatttatcatgat |
| | ccacaaactcctgtataggaaaagaatcagaaactcttgctcctagggtgtttttaaaatgaagagacttccctatcatgtg |
| | acaatagcaataaacgtaacatcattctatgggatccattagtcgaccttcatttcttaatgttgaaatcacagttttatgcac |
| | aaatatttaaccaaaatgcctaaacccaatttaatcatttttaagaaatgttaattattttgtcacttagatacagtttcctctcctt |
| | ttgccaataaaactataaaacagcactaatataaaagtgtagttggctatttggaagaagcaataatcatgccattcctgga |
| | gcattcttttatactttggaacaaaatattccatcactggctctccagattcatgagctataatgcctcatatattggaggaat |
| | gggatgtaaaatgggatccaagatgcgtaattgtttacagttaaacacagatgcgcatatacacagggactacagataat |
| | tactttttcctattatgtattaattcttcagaaaagcatgagatttaggcactttcggataatagcttgtttctcggaaagaggc |
| | aagggtagtttccttattctctgagtatcccattttgccaatttcctgtttagaaagatacttgaggcatattatccatcaacgt |
| | atctaggggattcagctggagtaaaggtggtagaatagaagctaagaaggaactggttcgtttattttcaatcctcacatta |
| | tggcaatttttgatttccttgtaaaagtctatgattctccctcaggaaacattgtccacttcctaaaaaaatatactaatttctaa |
| | tacaggggtttggaaaggggacaaaaatgtgcagggaaggtttgcgtaagcaatggtggaatgggttcaacagacac |
| | ctgtctatgactttatcctggagaatgtgtagtcctcatgggaaagttttccagtgggatagtgattaagatggaaaaaaat |
| | gcccaaaatatctttaatataagaacaaaatgggccaaacacgtgtctttgggtcactggtaatctactgagcagtaggac |
| | atcatgacataagagttccttttgccatccgaagaaaaatatttaaaatcctattatttgtggttttaaaaatgttataatgtattc |
| | attataaacactaaaatgactttctggataatatagtatactgtgagtaattattttgattttaccatattcttttttagttctcagaa |
| | accaaaattgtcagatatgggatacttgatttaatctgtatttgaagttttctcttttttaagtgccaattttttaattaaattaaatt |
| | aaaatctctctctcttttcccaaattatatacaatatctactaattatgttttcttcgaaatgtatcttagcttcataatgagaagtg |
| | agtgtgcccatgaaaaatttaataggaagttatgttttctcttccattttctgttgtgattcattatttttgaaaataatttactttca |
| | tttgctcacatttgctgtctaaagaaaaactattcatctggcacattcatatttagtagtattattaaagcagaaagcataagtt |
| | ggaagtataatatctaaaaatacaaaatgaagtattgtaccttgatgtttattagatcattaagcaaaatatgattctgccctg |
| | cttaaatcatttgattataattatccagcatataaaagaatcacagtagattttcaataggaagagtcctataatattaggtatc |
| | caccaaaaacattgttcaagtaatatttccacctgaaagtaaatgattgccaatgctttttttcagagcatataaaattggcta |
| | ttcctatttgatctcgttattgtcctggcatccttattttgttaaattttaactaggcaagggctatgctacaaacatcagttagtc |
| | cgctagtttcctgataaataagtacaggtaattaaaaagtgaacctaaatatccaaattataccaaagggacatatatagaa |
| | ctttttaaactggatctgcttctagccagttcatattttggtcacttactaatgtagtatttcaccataaattatgcctagattgga |
| | gcatttacaggcactctttatctgaaaattcttaagtgcatgagttgtaacagtttcacatagtgatcatctcactgttctaatt |
| | ggtgactatgtactacagttaggttgatcgtaattatgaccttaaatgaagctgaatatttttatattcctaatttgattaattttat |
| | tttatgggcttttacacattttaactgctttagtcaacatattttataatattatgacatttgcctagaatgtaattttaagaaagtc |
| | atttaattgatgttatcaagaagggttttataaatcagaatttcctgcctatgtttcaagatcattgcttaaagaaaactttttgtt |
| | catgtaatattccaatgtgtatttagctttactctacattaaaatatgttaacaactatgaatataacttgaattaatactaaagtt |
| | catggttttgaaacatggaaatcaacaatatcataagcactatcttgaacctacaatatttgattacatatctagtctactaaat |
| | gttttaaattgataaacattgggtttacttttgaatcatcaaaaagattctttagagaagcttggcagaatggggtgcagaag |
| | atctggaatccaaccactgattcgctataacttttcacaagacgataaaactcacatttccttctctaaataaatactggattg |
| | gctgacattaaggatcaatgtgctgcctagattctttgttatttgtaaatcaagtaccacaagtggaaaagtattcaagtaac |
| | acatgtgacagatcctgtgctgctccgcttcagaagacagtggggaaggataagattgcattccttaaagaggccccatt |
| | catcactggagctatagatccttgtatacagagtgaaaagagggaaaacactgtcaaaatgatttagtaatagttttcctga |
| | ctccacagttaaactacagttcacctcatacactcacattaggtccgaataattggcagactggttttaagacaatactcct |
| | agttctaagagttgttcgtcattgcccacacaattcagaatcttaaaagatttgtgttactctgcaattaagagaaaatattgt |
| | gtgtattcttttgaatgtgaaagtaaatatcagataggaagtgttagtagttagtggttggataaaagagtcctcgcactggt |
| | cattcattcatttacccaaaatttattaaacacaaacattggtgctagagatacaagggtgacgaaaacatgatctttcctcc |
| | aaaaaattaagtctgatgagatgcattttctagaaacacaaaatacttttgaactgaaccttggaaaaagtaaaaacttgac |
| | ctttcaatagataaatatttggctttaggaaaaaggtatcttaattctacatcagaactaaggtagtgcactaaaatgaaagg |
| | gagcaatgttaattcttctacttttaatgtgatttaaataagagaaaatacaggaatgtcttttataatttgaaattccagagaa |
| | aatgaataaaaaagcaattaaaaaaaacacctcaacatgcttctcattttcagccaagtacagcaaactctgtttgatattct |
| | ctgattttaaccttggatcaaactatttggcaaattgctaatttgaacaggctattgaaaacagacagtgtatctagcaattca |
| | ttcattcattcaatacttaatgaacagcattttggcaattcaaagcctgttccttgtattgaactatattggtgtattatccatatg |
| | gcctagagtacatgtgtattattcatattatccatattggtgtattatccatagggcctagagtacgtggcttctgcccccaaa |
| | tgtcttacaagcatgttaaagtacccaaaatccatccaactgaaataatttgacccatacattatcaaggttcaagatgtgtg |
| | ataagccatgaaaaggttggtgaatagcctagggtgcatatggtgagtgaagatggatggaagaaattaggagtccag |
| | atttattggggagactttatgaaagcaaggaatcatgattgttgcaaaatagacagtgaagaaagaaatggtgtggtggt |
| | gaaagtcttcccaaagtcatttaaagtattggcaacccagatacttggcagcaggagtgataattgatgttacaggaatcc |
| | tatgtaaacttttgcacattttaatgaagattttctataatatgctgtctggtgacttctcccaatcactgtcaagggctagactt |
| | cattattttaagatgtctttcattcatttattcactcactcattcttttttttaagcaaaatttattaagacctgtaacatacttagca |
| | ctgatgtaagctctgatacagtcattcagtaatgtcagcctgagcatttgcagaaccctgaatatggatcctcatttttccctt |
| | ggggtcacccgaagtcttaccccgttctgtgtttacagtgtgaaccttattcttataaggaatattttttgtttcttattgtggatt |
| | gtctgtacttccatcagatgtgactcagcttcagtttttctgattacctgtgttattttccaacatgttgcaagtgataagatcag |
| | tattatcaggtccctaatagcataacaggttcgtgtgaattagatatattaaaatgagaagatttttaagtcattttttagtcaat |
| | tacgtgtatagaaacatagttatacttatctcagaaagattccaattcaaaggataggaaattagaaaaacatgtaatgtttc |
| | ttctgagaacaaatatattcaaattttaatgacaaagatattaggattatttttctagtaatttgatatacctgataaattaaaata |
| | agaaatcatcaaccttcaagcaccttataatatataatctttgatttaacttttttggacaaattaaaaaataattctctaactca |
| | aagcaggcagaagtatctaatatttatgcttccatcattagtatttttaaagtacaagagacaaaagcatttttaatctcatgta |
| | tatcatacatcaatattttttaagtcatcagcatcactatgctaaggaaatattttatataaagaaaattattttcataaaataaa |
| | gaaaccatctttctagagaaaatccacataatcctatgcccaaatataaatataacatttacagtactgactacagcatccat |
| | cttattctagtttaacatacttcagtgtgcattttatcatgtgcttacctcatcctttttataaagtatctcttatcagctcaaccatt |
| | tcctaatgttatgcactaatggtaataacattgtaacaggtccgagaatgctgtggcccaaactactctgattcctgtgtgg |
| | cactcacacctggccctgcatcatttctagagcattttaagaccaggttaaagactgggatcccttcatgtatgctgatccc |
| | aagaatggaccgacctggctaaagtttattgcatttcaagatataaaacaacttctattatatttttttttggtgatatttgattt |
| | aacctaaaggaaaacaaaagaacaactaaatattcatttctgcctgtactaacagggcaggtaagagtgccagagtaac |
| | aagtagtttccaaatgcacaatgaaacacaggagtgcattggccaaagagaatgcaaaatatcagctcttgctatatagc |
| | taacaatgtgctgctctttttggaattagaaaattatagaatatatttaataacaatctggtatatgttttcatgtcaatgaaaag |
| | tggataaatttagatggttgctgtttatgtgcatttgatcaaatcttttctgaatttgacatgaaaatacacttgtgcagctttcat |
| | tggttgggtcacaattttagaataaaacaaactatttgaaaaccatttgcaaactaatgtacaaaagcaagatcgcagatg |
| | attatatgactctggcagcttacataagctttctgcaggattttctttcagaatctctatacataggctcaaacagaagttattt |
| | ccgttgttagcaccatattttaaagaaaaaaaatactatggtgttgtatctaatcttgtgacccctgacctttaccaaagcgg |
| | attggcattatgtttaagttcttaaattacagatcaagaaaatgcatacagaagatggggggggggcacacctaattaattt |
| | ttatatttagattaaagaaaataattaaatgtgtttttttgtgggattgattttcagaagctaaatgcaactagttcatctgaagg |
| | cagcacggttgatattggagctcccgccgagggagaacagcctgaggttgaacctgaggaatcccttgaacctgaag |
| | cctgttttacagaaggtaagcaaaacaataacatatgtggtcttgagtatcctcttttctacccattttttcctatttatttaaatg |
| | tctgtttatttgtctaccatctattatctatctatctgtatctatctatctatctatctatctagtaatcatctatacctatccaacaac |
| | tgtacatttatttgtttttttttgcatttgctgttttgaaaaaaaatgcaactttttaaaaggcaaagtttaatttatgtaattagata |
| | ttttcatttttatgaatcatttttaactctaagaaattattaactggcttttctgtggccttctaaaatatcttacaggagagaaagc |
| | caaatcacacacatctctctttagtttaaaaattcaataaataagaaagtgagagaagtaatttattatgtactattttgtgatat |
| | tataatgggtaataattgataagtgtacatttaaatttgtccttgactgaaacagctcctatttcagtcaaggtcaaatatttttt |
| | attatttctgaaaaaagatagatcataaaaatgccaaaatatactatgagtcatatgatatggggcaatatgtcactggagt |
| | aatcgcaaaaggattttctgaagaaagctaaaattatgtaatttgaggtatggatcagttatatattgtaatagcaatgctgt |
| | gtatcaaaccaccaaaaaccctgggctctaagctgcttttctagttttgactcctatttccttctgtgtaactcacagactttct |
| | tgtcactaagttttacttgtatcattgtttctctattcttacagcttcattttctacatatgtctcttatatatccttcaagatctagt |
| | ctcaaatccatttcctccataaagctcagaaattaaagtttaccagaaaactctcataatactttgttttgtgataattgttgctttc |
| | cataactatagaattgtagacaaattgccccaacttaaaatgtacattctttgaggacaaggctatgttttacatgttatagta |
| | ttacaatttgttctatgcaattttttgacaatagtagatactcaataagtatttgttgaagagcctttgatctagcaatccagaaa |
| | ttatacaaaggtgtttattggattgttattgataatggccagatttaaagcaaacgaagtattcaataatggtggaattgggct |
| | gggcacagtggctcacacctgtaatcccagcactttgggaggccgaggcaggcggatcacttgaggtcaggagttca |
| | agaccagccaggcaaacatggtaagaccccatctctaccgggcgtagtggtatgtgcctgtaataccagctacttggga |
| | ggctgaggcaggagaatcgcttgaacctgggagacagaggctgcagtgcatgagccgaggtcaggccactgcactc |
| | cagcccggacaatagagtgagactccctctcaaaaaaaaaaaaaaaaggtggaatagttatattaattatagtaatcatat |
| | ttagagaaatattatgaaatctttcacaaatttatttacttataataaagatgggaaatagttataccattaagtgaactaatca |
| | gaattcaaatatgtaaagtgtccatatagagtggaattacactcataggataaggacaggatggaaataccaacttttggt |
| | aagtttattttctttttggttcttctattttttatatattgtgtttttgtaatgtaatccattatagtagtgctataaacataaaaata |
| | aatatttattaaacaaatgattaaaaagccatatagatgattttaagatagcttttgtaagcggaagctatcttaaaaattaatgtta |
| | tttacaatgtattatcaggtaataatgtaaatgaatctcccaccaacacaaatatacctaatcaaagagtaattttttgtcttca |
| | tttttttcccacatattttagactgtgtacggaagttcaagtgttgtcagataagcatagaagaaggcaaagggaaactctg |
| | gtggaatttgaggaaaacatgctataagatagtggagcacaattggttcgaaaccttcattgtcttcatgattctgctgagc |
| | agtggggctctggtaggtgatgcatgatccactccttcacctttcatctgaaatcttttccctttcccttcaatcaactcatatt |
| | acccacttttaaattaaggtgtttgtaagaatgagaagaaatatgtgtgacgtgtttagcacatatgagaggcttagtaaata |
| | gcaatttttgtcactctgtctggagtagccctcgggtggaaccaaactcagatcattatggtttcttataatgtttaaagaag |
| | gatctttctgactttcagtcatcagaggcagttcttattaagactggttatgtagacatgatgtaggattatcagctaaatatc |
| | agactgaagcacgatatttccctgacccctttgcaggtgagaactagagtgcatgggtgccggtaggagcgaactcca |
| | ctcactcactgctccacccctcacaggagggggagcgcaggtgactgggtgcaggagccaaggcaaatgcatttgg |
| | gcactgcaagagtgaactccataccggccccacaggagcgtctaggggagggtgcctgcgatccttgaagccctaga |
| | ggaagtgttacagtgcccttttagctttgccatccatggatggcttaaatgttaacagttcagtggagggtcagagtgaca |
| | gccttttgcacccacacttgtggtacccaagttcatgtccggcgtccaggaggaatgagtttgtacaaatgacttgaagat |
| | ggtaaatacaggggattttattgccagcgaaagtggctctcagagggaagaggagctgaaaggagatggagcagga |
| | aggtaatcttcccctggagtctggccatccccagccagactcctctccgaagctatgctgtcaagctgtccctctgatgtc |
| | aagctacttctctctaatgtccaactgtagtctctgatgtccagctgttcctcctgtctgcctgctgagttctgggctttatata |
| | ggcacaggatgggggcagggtgcaccatgggtggttttggaaaaggcaacatttaagtgagaaaacagggatgtatat |
| | tctcactttgggccacggttccaggcttgagggtggagccctcgccaggtacccgtcctcttctgcccagaatttctctgc |
| | ctcttgttcctgtcaaaattgcttaacataaactccatgctgcaggggactcctctgtcttcttcacactgattcgctattgcc |
| | aaccacagtgaatgataagaagtagactcacttaattactgactagcaaaaaaatgatggcattacaaacttatgtctgatt |
| | tcattcaatgaaatgatcaactggatcaaaatattaatataatgaaaatgatatgacctattttcttaattggtgatacaaatgt |
| | ggttgcattccttttactgtttcaatttaattaataactagagtgtttggtgagttgatttcattaggagaattactgcattggatc |
| | tggaggcctctaaggcgaattctgatttgactaagaatcctgtgtcctgccatatactcagtttaaagaggatcagccatg |
| | ctttattttctttacctttattattattattattattttttagacagtcttgctgttgcccaggctggagtgcagtggtgtgatctcga |
| | ctcactgcagcctccatctcttgggttcatgccattcttgtgccttaacctcgcaagtagctgggattacaggtgtgagcta |
| | ccacacctggctaatttttgtacttttagtaatagagactgggttttgccatcttggccaggctggtctcgaactcctggcct |
| | caagagatctgcccgtcttggcctcttaaagtgctggaacgacgggcgtgaaccaccgcaccttgccagacatgctttc |
| | taaagccaagtagagagagaactatgaagtctcattagtgactagtacctttgctgtaggagctctttgttctcagttacac |
| | ccagtcagtgctcaccaaattgcacaacgtgctggcacagtggctggctcctcaggggtttacagcttcagctataagc |
| | aaagcccagaaacctttaggtccttgtatggagctctggttacaagccctgattcttgttatctaaaaaagaaaatgttcctt |
| | tgtctttaatccaggctgccaggttttcctgataatttttccgataagaagatcaagttagataaatagtcttttcattctggaa |
| | gcctcaggagttcctgcaaatgagttacccactctttcccaagggctctggaaaattctgtcaaagggaatttccaaacgt |
| | acacccacccgcctccacacacacacagacacacagagagagggagagagacaagaaagtgagcaatgacaatcc |
| | tttcctttttctgtaggctgagggacctccctgctttatatctgcattactagaggatgcattccattgagtctgcactgaatga |
| | gaccaatctactcccaggcgttccactgcctcctgatgtagagagaagcagctggcagtctctcaaaaattttaagctctt |
| | tgggggtacactgagaccaaaatttaaaaattactgaaacccttggttgactgaaatgcccagtcagcagtcatttatgat |
| | cagataatgataaagtaaaattcagccatgggaaacattaaaccttccagccttaggcacctgataagagcttgcatcgtt |
| | tccttttttaagaaatcatcaattagagactgtttctgatcataaaatttaatagaattttttgacttacaggcctttgaagatata |
| | tacattgagcagcgaaaaaccattaagaccatgttagaatatgctgacaaggttttcacttacatattcattctggaaatgct |
| | gctaaagtgggttgcatatggttttcaagtgtattttaccaatgcctggtgctggctagacttcctgattgttgatgtgagtat |
| | gctgcactttgctgctttattcattggcatatatgtaatagttctagcaatggtgcctgacacagtgtaggcactcagtaaca |
| | ctgtatcagcccaaatataaattatgtttctcatttcacagtgagaggatgcctcaaaacattttttaccaatttaaatacatat |
| | acattcatagataaaaatcaaatgccatcatactatacttattcacttaatttcaaattaatatttaaaatctcaagttatgcaaa |
| | ataaaatatgaatttagaaattttgctttttgcacactcacatttcgcaaaataacttgtatttaaatttttcacaggcatctttga |
| | cattagtatgtttgtcatcactaaagcctgttgagtttaggtcacacagatgaatcattaattacaaagaaatttgaaagtcca |
| | aaaagcaagagacaccacttgatttgtatgatatagaagcaaattggctattgaccaagtagccaaagattttattaaacc |
| | acattggtgttgaaataaaataagatagagtactaaaatatgagggtttttatataattgaatatgaggcaaatctaccatta |
| | aatgtactactactattaaatgtataaaggttacatgcagaattacattaacagtctctggcaataaaggaagacaataaat |
| | aatatttagaactacataagtgtggacattacaaacaatagaaaatgcaccaaaactataaccattcttttatttgtataatgg |
| | gattatgcatgatactatttcttttctctattttctgtatgtacttatcataggttggtaaatccataataaaaatatctgatacttg |
| | atatatctatgttaggataaaagtatcaagtcagcactgcttgaatataaggaaactcttcagagaaatctagttgtcctgca |
| | gctaatgatcatattacccaaagtactctgatatttacctttttagatttaagaaaactattatgatagtatatgaaactgatcaa |
| | cactttgccttaaatcaaatatgcttattgctcatctatttcattatgaaagatacaaatataaataagtcatttttctagtcctgc |
| | agtagcttacagttgaaaagtgaggacagctgcgtacacagtaagtcgacacctgtattacaagtgccacctctttacttg |
| | aggaaggaggaaaggcttcaatagggaagtggagtgtgagctggagcttgagagatgtgaatgctagcaggcacag |
| | ctgagggaggaacacggattcgttaaaacgttggtgcatgacatgcagggcgggttccagaaacaagtagatagggt |
| | gaggtaagcctttgtaatgggatgataaggtaagaaagataagttagaaaagatctgaagaacctgagatgccatccaa |
| | ggaaatttggacttattatttaatacagaggaagctattgaagaattacatatagggaagtgacaagacctgcttgttctttt |
| | agtgagggaagttaggtggaggtgagaatgacggaatagaaaggagatttatttagagatcaaaacaccaattaggag |
| | attgctgcaatgtcccagaaagagaaggcctatatgtatcttcttttccacatttagctacacaagtcacataaaactgaata |
| | ttttacaacttcttttcagccagtaaatactaccccattcaaaatattttcctctgtctaacttttatctttcatcctttaacttatgct |
| | tatctctttttggttctgtcttcagagaaggtaaagtactacaggtccttatatcttaaatacagaaaagcttcacaactcatg |
| | ataattcagtaactatttttcaattatctgttaaaaagggacttacaaagcctaagagtttggattttaagggaactatatgaa |
| | ctatgtaagacataattttacaactcattgttttctgtattcaagaggcttcactttcaaattgcatgtgcaaaattattttgaata |
| | agttgttttttgtaacaactttcaatgtgcttcacttattttccttaaaaaatatatttttcaaatatattaacaccatactcttaaaa |
| | gctgtattgcatatttatttttatttatctgcttttgaaattcaggtgtactttagaacaaaatagcttatataattttaataatttttc |
| | tatatgttttcaaggaaattggacatgtgtatgtcccccgaccgtttttctttttctttttagctaagactttataatttttctcaacta |
| | cattagtcaactgtatgaataactaaagacaacattgttcttgcaatttctaatttatcataaaatctcaactttttttattcactaa |
| | ttttgtctgacctaattaatgatattatgcccttcaaactgaaatttacaaaagtcaaagctgctttttagaggcctattccttttt |
| | aaatgtgttcatgctcatattcaccagtggtttgtatagtttacttgtgtatcaaatgttactttccatttcagatctgctcaatatt |
| | attagaaatgatacagaaataagttttacagatctgtagaggaagatcacatttctctctcttttttttctttacttttaattttttaa |
| | aaacatttcctaccaagaatcttgaaaaagagcacatatatgggcttcttttttataagtgttcgcagactagtatcattaactt |
| | caccctgggaacctgtagaaatgcaaattcttaggcctttccccaaacttactaagtcagactctgctattggtgtttttaac |
| | aagacccctgggtgattttgaaactcatgaaagttcgagaattactgattcattgcatagagcaaggctgaactgtgtaga |
| | catttttatatgtaaataagaaaattgtgttgctttttctgtataggtctcactggttagcttaactgcaaatgccttgggttactc |
| | agaacttggtgccatcaaatccctcagaacactaagagctctgaggccactgagagctttgtcccggtttgaaggaatg |
| | agggtaagactgaatgccttagagtttgtcagaattattattgagagcagactgacactttgtaccatggaaatgtcaaattt |
| | atggagaatttgtgtcttacacattcatactgacatagctaatcaatcaaaaataatatttaccagatgcccataatacttggc |
| | actgctggagtcactcacagagtagtatattgccagagggattgtttctgattagctagattttcacttcttggaaaatctcta |
| | tagttatgctgctgatttgaatcaagattatttatgttcacttcatttataaatgtgcaggaaatcctactcgctgtagtttaagc |
| | ctaccaaatcattgctcatcatttcttcactactccgctgtgatacactttgagccttttgatgtttgaatcaggccttttagttct |
| | taaacacaggctgaaatggctaaaaagtaggtcaactggaaatctaacgctcatttagaagggtggtacaaaagaaca |
| | gaggagtttgtgctgacatttgtcgtcccctgaggcacaaaacctgagaccacataccctcaccacctagaaaatgatg |
| | atgccttgtctcagttgttttagctggttcaaagaggattttaaaaaaatgatactttttgtgatatttgaaaataagttgcttag |
| | actttatctgcatgttatagtgatactagctcatattttctaactaagaaaatagttacttagactttatctagtgttacaatcaca |
| | actagagatgaatggtgtgtgtagatgtgtgtctgtatatgcatggttacatagaaaagtgttattagcggtaaaattcttttt |
| | actttaccaattagaaagaacagtttttgcagtagaaggcttaataaacaaaaggtatcaatctttcagtaccagaatactgt |
| | ttatattttctgtgtggaatttgatccccaagtggtctcttttactctcaaattttggacagcaaattgtatggtttgtatgatttttt |
| | gaaagtgatgttcacttctatattcatgccactgtttatactcttaattatttttggcatttgctgttagttccatcctttgaggtaa |
| | atttgctacatgtgtgttattacctcttgagaaaacattctccaatataaaattcgttgtatactcttctgatttataattttaaaatt |
| | cttagttggagctaccagagtctagtttctacccaatattcaactttgaaacagatttttttaatcatttgactgttcttttaataat |
| | gtttaaaaataagtaaatatttgttgttggcttttcacttatttttccttctcatcctgtgccaggttgttgtaaatgctcttttagga |
| | gccattccatctatcatgaatgtacttctggtttgtctgatcttttggctaatattcagtatcatgggagtgaatctctttgctgg |
| | caagttttaccattgtattaattacaccactggagagatgtttgatgtaagcgtggtcaacaactacagtgagtgcaaagct |
| | ctcattgagagcaatcaaactgccaggtggaaaaatgtgaaagtaaactttgataacgtaggacttggatatctgtctcta |
| | cttcaagtagtaagtaatcactttattattttccatgatgtgtaattaaaatgagtctaaagtttttcttcctcataatgagatatc |
| | cacctgttagaatggctattatcaaacagataaatgacaataaatgctggcaagaatgtgaagaaaagggaacccttgta |
| | cattgttggcagggatgtaaattagtatagcttttatggaaaacagtatggaggtttctcaaaaaactaaaaatagaaccac |
| | tatgtgatccaacaattccattactgggtatatatacaaaggaaattaaatcaacatgtcaaagagatgtctgcactctcac |
| | actcactgcagcactattcacaatagccaaaatatggaaacaacctaattgtccatcaacagatatgtggataaagaaaa |
| | gtgtgtgtgtgtgtgtgtgtgtgtacatatatgtatatgtatatatatacacacacgtatttctatatacacacgtatagatatac |
| | actgtatatgtatatatctatacacatatatagacatacacagaaacagtgtttgtgtatgtgtgcgtgtatatagaagtagtc |
| | agggaaggggcagagcctgtggcactaagaaactgagaaaatgtacaagacttttgttttcagaattactatgtccgcac |
| | aacagaaaaagtatttcaaaaagtaaatgcgcttgaatgtatttgttttcagtttaggaaactgcttctttttgtagagtgcctt |
| | aaaatagtatgttcaacaatattaaaaagattttcaaaaataagccctcgtgattgatgattggtaataatcatttaaaaactt |
| | attggatgtatatatatgtgtgtgtatacacgcacacacacacacacacacccctatagacatacacaatgaaatagtattc |
| | agcctttaaagaagaaggaaatcctgtccttttatacaacattgattcacctggaggaaattaagtgaaataagccaggca |
| | cagaaagacaaatgacacatgatgtcacttatatatggaatctaaaaaacacaaactcacagaaacagaaagcagaatg |
| | acaatcaccaggggctgggggatgatgggagatgttggtcaaaggatacaaaattcaattcgacaggaagaatacatt |
| | ctgtagagctattgtacagcatggtgactatagttaataataatatattatatacttgaaaatagctaagtgagtagatatgttt |
| | tctcatcagaaaaaaataagtataagaagcgataattatatattacttagcttgagttagccatttcacaatatatatatatttg |
| | aaaacatcattttgtacaacataaatatattcatttttatttgtcaattaaaaaatgaatatatttttgaaaagcaattaaaataaa |
| | aatgcatatacattttaggaactctatatagatgcactaaaactatataaaaatgatataatactatacaacaataaaataaa |
| | atttttcttcctctgtgtttacaaatacttccttaggcccatctgcctagattcctcttaccatgattgaactatcttttctgcccca |
| | cgctggaaacatgatggttctaaaaactttattgtctccctgactatgcatttggtagcatagccaagtcctttgttactggg |
| | agtttaatctaggcactcattgttttcctcccttcctactctgaggaaagaagtgctggccccaaggggggttgaaaaggg |
| | gtgtgtgtgtgtgtgtgtatgtgtccacacgcgtgtgtgtagatagagaaagagagagagactttcaaataggaaaattg |
| | ctctcttgcaaatgaaaactttccaattaagactattgtgtctgctatgcactcataataattcattcagctattcaactgactg |
| | cagtattaaatctccactagctcctggacacaatccacttacacgatcctcaagactattaaaatagtcaggaaagggga |
| | agagcctgtggcactaaggaactgaaaaaatgtacaagagttttattttcaagatcattatgtcaacggagcagaaaaca |
| | aatatttaaaaaaggaaatgcagtagaatatattgttttcagattaggaaactgcttcctcttatagagtaatcacctcaaaat |
| | agtatgatcaacaatattaagaagattttcaagaataagctgtcatgattggtgattggtgtaataatcatttagaaaagaat |
| | aagtagaaaggaagcattaagataaataatgcagcatacttttgagcttgtctcatgctgctactatacacatgaaattttttc |
| | atcaaagttcatgatatatttttatataaacacatcagagtcaaagattgttcatattgtttttatgatagcatattgttacagtag |
| | atcattatttaattatatatgctaaatatccacataagatgttatagaggaatataaatttgaagtattttcaatgcatatcgcaa |
| | aacattgccccaaaagtgaatacaaatttcaagcttatttatatgcctgtattgaatacatgtcaaatagaattttgatcaatta |
| | ttcaatttattttctaaaattataattttgggaaaaaagaaaatgatatgacttttcttacaggccacgtttaagggatggatgg |
| | atattatgtatgcagctgttgattcacgaaatgtaagtctagttagagggaaattgtttagtttgattaaatgtatatttctacaa |
| | tattgtaatttagtgatattgtcaataaaataaaattatgtgcttaatttataaaacccatctatattataaggataaaatatttaat |
| | catactatttctttcaaaattatcataggatgattttctctaatcactctgtatcttttaacatatcttttctagtatttagcaaggca |
| | cctgacacaaaactttattgtatgtattttcaaaatgagacattttatttttggctctgatagtcctggtcatttgtgcattagaa |
| | gttctcacaggcaatattttttatctgtaatatatttcctccagcttttgatcttccttataataggaaggatatgactaaaaacg |
| | gggacaaaaataaacaatttagtgtttctcttgggaaagtgagattaagtggtagaagggagggacttccctaatctacttt |
| | atacataccagtactttgaattcttttctataattttcattaatttctcactatttaatgaggaatgaagtcacattttgaaaaaaa |
| | aaaaaaaagagattgatttctggtatgccagagcatgataataaagctcaaaatgctctttccctagcaccagcagctag |
| | ctttctgagtgaagaattcctgaggtttttttttttcttttttccacttcataaaaacagagagggagcaagaaagcatgaaaa |
| | gccctgcattgtatctctataagtgctatcaggaattccagttatgagatttttctgaatagtaataataatttattgattatcact |
| | attcactgtgccaaggactttctcacattatcccatttaatcctaaatgacaaccttattgtataggtgatactagctctatttta |
| | ctactgaagcaaagaggcttaatgcgttaaatgggaaaacaagtttttgaaccctgaccacaaataatggctcataccca |
| | ctttccacagtggttcttacctttttgattaattaattcaatgctctctccaccttccttatcaatagcttatatgccatgaaacatt |
| | ttcagtttcttctttaataacttagcagaccttttccgctgcaaaactcctggaatttccagcacattacaaaagatgaaagcc |
| | aattgagcactacatttatgaaaagttgctggatcttgaactttaattagtaaattgcatcagataaatgcaaatttaaaccaa |
| | aataaaacattatctacacacctaccagattggcaataccaaaaagtctgacaataccaagttttaccaaggataaacag |
| | caataagaacactcgtacaatgctgataggaaaaaaaatagttaaataatcctttaaaaacagttgggtatgatcacattat |
| | ttgagaaagttaaagatattttttaatactgcaattctactttgaacaacgtatcctaaagaaacttatgcacatgtttaggata |
| | atctatgtacaaaaatgaatataacttttttttgcacttgcaaaaaactgggagcaactcaaaaacagtagaaataggcaaa |
| | taattgaatactatatagtgatgaaaatgaatgaataccgccatatacaaccacatggatgagccttaaaaatacaacattg |
| | agttaaagaaactagacacatactataattctacttatataaagttcgaaattgacaaaactaagcttattgttcaaaactgc |
| | atactgaggtgttaacttgaaagaaaaagcagggacatcattaccataaaagtcaggataatgattacctccagcaggg |
| | atgatggagtttatgtttgagaagggtacaccaagggtttctgaagttgtagcaatgtcctgggttatggatttcacttataa |
| | aacatattatattttgcatttatgtattatgcactttcctgtatgtatattgtcttttaaaaattttaaaaatataattttacatcact |
| | gttaactaaactcacatacacaaataaaatctcatcgaagaatagcagttttacaatattcctgatattttccattttgctgtatttc |
| | cttagaaacaaaattatgctggtcataatcctctaaattgatttcataacacagtgggttataacttgcatctattatcatcatc |
| | agggattggttaactgagttggttagaacaatgtcctattagacctgtgaaagcttacagctaaggcgcaaacctactatc |
| | acacagttttctaaacaaaagtggattagacaagagatagtatcattgttacagaaacagtccctactgaataggataaag |
| | caatagattcattttcagaaaggaaagatcaacctatatacctacatgcagacctactacaatgattcttgcctatctaaaga |
| | aatgtattataccaaacccttacacttagcaattactactggccgccactgttctaagcatatttatatgttaatatagttaatct |
| | tcacaaccacactatgaggtttaagtttgattattttcatctcacagatgagaaaactgagtcagagaaagtaaatcttaaaa |
| | gttttgacatagaataatgtgacgctgacatctcttttgtaagaagaggaaatctttaatttgcatgctgtgttgggaactttg |
| | cttagaaaggaaagtgcattcataatctgggcatttgttgggtgaaattgtctataatcattcagacttctatatggttatttca |
| | ttttcccaggtaatgaatagtcttgcagaactcttcaataagcatgtgagatttgaaggttcataaaatctgtttagtgtttggt |
| | ttattttcattccagagattaaaacatgcttagataattaaaaactcactgatgtactttttgtgaaacaagtactagatataat |
| | ggttacaattcttcatattctttaggtagaattacaacccaagtatgaagacaacctgtacatgtatctttattttgtcatctttat |
| | tatttttggttcattctttaccttgaatcttttcattggtgtcatcatagataacttcaaccaacagaaaaagaagataagtatatt |
| | aaaacttcatccttgctctgaaatatgaactaaatatttcatactctttcctttagcctccaaaatgcaatcaccaaaaaaaga |
| | atataaaattcagaaattattttgagacatttgataatcgataagcttttaagcaattaataattcagatagcatgttttttgatatt |
| | tttagtctagaaatatgactaatatggcataatttatatattgaataaaggcatctctataaatacagatattagtaacaataga |
| | atgaaatgtgggagccaattttcacatgattactaaggtggattttatagccagcaaagaacacaattttaacaagtgttgc |
| | tttcatttctttactttggaggtcaagacatttttatgacagaagaacagaagaaatactacaatgcaatgaaaaaactgggt |
| | tcaaagaaaccacaaaaacccatacctcgacctgctgtaagaataacatattttcattgcctgttaaaactatattacctaac |
| | cgtttcacagcccgaatttctagaaactagttatttttgtggatttgtaacacaaagttttttaccttaacaatgggactagcta |
| | gcctaaatagcttgaaaaatgtactttacatatataatatgtataaattatataatgcataacatattttatatgtaaacatataa |
| | aatacatagaaataaaatttgctatacttaagtgccagtggtatcatacaagctgatgtcattaagacacttctaataacatc |
| | aaaaataaaatacatacatacataatgtgaaaatattaaatgttctcagagtacagaggagacagatcggaataattggta |
| | cgtcacagattggcctcagtttttgtccaactctgcagattgaatggaatcattaatgaaacaggccacaggttttgctttttt |
| | ctggttaaacaaaaaaaagacaaacctcatattttcccctactatcccacccttaaatgagatgatatcattctttgtagggc |
| | tttttattggctcttccaggtgtacatttgccagtgatactgttcgttcagtttggctgctgcagggagttgctgccaggaga |
| | atcgctaagtttttctatcactcctgaaggactagctcatatattaagtctcagaaaatcttccccaacgtatacgtggtataa |
| | aacacttcagtgtttctcagaaatcttgactctataaatctattggtgacaatataaaacagaccgtaattaagtgttcagttg |
| | gtaagccggccaataactcaaagaaaatggatagctatattgggtcaaacacaaagggtgtacaacttgagcctagtctt |
| | taggaaataatacaatttgaatgaatagagagagaagcagagaacatttactgtatgagaaaatgtatacttcatagccat |
| | atagacaaatatatcagtgcagaatagtgatgcatttgaattagtgagtagtagacactggttttccgagttacatgagaca |
| | aggttaccatacgagtctgaagaaatttgttctaattaagcaatacaaatgcaatatagttaacagaacagcctagtaatgt |
| | gaaaagaaagattttagagagtttaacctagagactggtgtggaacaatattagaggcaaaataaccctcggccataga |
| | caagaagataaacccttacatacaagaagatagtccataatctgtgtccaaccagcaggactggaactactccaggagt |
| | gaagttagccaataagaagactcaattgggatgaaacacaggaaaagagggaggatgcaatgaaaaaactgggttca |
| | aagaaaccacaataacccatacctcgacctgctgtaagaatagcatattttcattgcctgttatgaaacacaggaaaaga |
| | gggaggatatgtaaataacagagaatctaaaatataagctagttgatattttgtgaaactgttggttccactatcatatactg |
| | aagtcatatgaaggcactgggaaaaatagtgttagagcctatgaaatgtccagactgaaataaggattttagcattgtcag |
| | aacaaaattcaattgagctctgaaacacagattcatttttgaaaaataattagaatagagaaaaaaacaaaattctcagaat |
| | gaggccttgcatacttcatcaagatataggaagaaataaatcaatgaagaaatgagcttgagtttgtttccatcaaatgac |
| | atggatttacctgtagtggtaggggtgtgtggaaaaagttcaacacattcagctagaatattatcagtgtcaatttggcaatt |
| | tagcaagtaactagtaaaatccatttattcctgcattgacaatatgtactatgtagtatgctaagcatttgaacttaaatatcga |
| | acagtatggagtctagttaatgcaacggatagtaatcaaatagtcctgccaaaaaatggaagtatcccagaaaaaaagg |
| | gatactttcagctgtgagagctgattagggggaaggggctgattaatcagggaagttagggaaggctttattaaaaaaat |
| | atactagctgaggatggaaaaagaatagaaagcatcaatagccagagtgggatgagaagagccctgtagaagggga |
| | atgaatttgtgaaggtccttatgtaggagggctggtgagactggagtgcagaaagtcaaggttcatttgggacacactga |
| | gaataaagaggttaggataagcccaaacttttctgggccttggaggccgtgttaaggagtagttttcatcctaagagcagt |
| | aagaaaccgttaacgtggacccagtcagtctgggctttgtggtgatcactcaatcagtttcacagaggccgtgtgaatac |
| | attgtagacttgttttggagctatttcagagatggtaggtagcctgaaccatagcaatgtgcagattaataaaagtggatgg |
| | atttgtgagctatcaccagagtgaaatttaaaagtttgtctattaattgaatatgggaactaaagaaggaaccaacaagaat |
| | gactggtgtctttctgctttgcacaactggataaatactgatgtcatgcaggaaatgaagaagggacagaaagtggtgag |
| | aaaattggagatgctagtttgcagaatttggcaaacgagtcagagtgagagagtgagaggaaggaggaagggagag |
| | aaatgatgaatatttagaagtagcaaaataaaggtttcttaagattcagagattaggtttaaaggaaagcaaaaggaatttt |
| | agagaggaaaagatcgaagacagagggaataattacggcataaaaatgcacaagatgtgggacaaggacatagtgg |
| | tctagggtagctttagaaagaaaaaggggctgagtcctctaatgaatttggagtaatatatgaaaagaacatggaaattaa |
| | aataatatgaaatgcaaaaggaaacagaggagtttatttaaactgtttaaatttaatgttctaaaaaaagtaaaaatagagg |
| | gcaagagaatggaaatttatgagaagtttggaattatctttggagcaaatgaaggacaaaggattgctaattgttaaatctg |
| | aagggccaagatgaagttagagaacataaatttttggtgaataagatcttcagaattatacatcttgttccagcatatttgac |
| | accctaggatttaaatgggagaacagaacacagagactcgagactggagttgtacattgagatgtctgtctcattggaca |
| | actctatgaacagggaatctaaacagttttttattagtcatggtgatattaaaattaagaccaaatttctgcttttaagatatttt |
| | gaacttactatactctaggagccatatctgagagaaaaatgatactgctcctgcttttgaggggcctcaaaacaagtgga |
| | agggaaagaaaactaaaattgaataagagcaaaccatttgcaatacaatgccatacattttatgatcaatgaaagcactc |
| | agttttctgagagcactaagtgctttaaactcaaaactgagttagaattcatgagacagagaaggagtgggggacatgtg |
| | ttttagacagaatagtagacagataaactatgtaaaatgatacagtagaacctgcctaagcttctaagagtggtaggcag |
| | gaaatatcagagggtggaagtaaggggaagatgccagacttggaaagttaaactacagtaaattaataattaataagga |
| | agggtttgaactaaaagtagacacatttattgggttgaaaaaggccctgagaagacagatccagctggagtaatagaaa |
| | tctagttcagcaggccaaatacgcattcagagaaaagagcaaaacaaaatagaacaagtagtgtttgatgtccaaaaat |
| | caccattggaagtaatagaagcctcccaatagaaagagggcagaacactaagatgtagaatccaggccactaaaagt |
| | gtcaggatctgggaaagcaagccattaggtgtatatgtagcagagtattagtcattctagttgagaaggtagagaaaggc |
| | agcccaacagaggttaagtcaagaccagatccctagattacctgagaaacaaagcagatacgtgcaaaatggaacaat |
| | acagaaaccaatgatcagaactggtttacaagttggggacttcatttcataagcaagacataaggcaattagtacttggaa |
| | ataaggtccaaatagactagggcaaagattgaatatttccattgtgactttttaaaagataattttattcttacagaagagtta |
| | ctcataatgaatactctaatgaatctatacacagtgtcctcttgttttaacatcttatgcaaccatagatcagttctcacaacta |
| | agaaattaatcttgatataataccattaaataaaatacagaactgagtcagatttcaccagtttttccactgaagccttttctct |
| | agaatgatgatttttaaaacatcttagctgaactttaaaatgaaatttaagatgctgtagctttagtgagagaatataaagtca |
| | gaaatcagacgaaaaatttaaaaagagagaggaaaacttggagaagtatttatttattagttgcttaaagtaaaattaatac |
| | cctcccaacacatgggataaaaaattttattacatgacaaatatttactaactgtccgtcataacatgatggtgttctgtgca |
| | ctgagaacataatacgtgagtttataaaacctggtatcaatgtgagtataaataaaacaaatacatttgaatacagttgaata |
| | tacaatatacaaaattttcttccaagtataaaacgaaaataaaatacactactttctttaatagaatagaacattgtaataatgt |
| | tccattgcatttgaccctcacataaatgctatgaggtagcattaagagataagatttgaggctgggcatggtggctcatgc |
| | ctgtaattccagcactttgggaggtcgaggtgggcagatcattaggtcaggagtttgagaccagcctgaccaatacggt |
| | gaaatcccgtctctactaaaattacaaaaagtagtcgggcatggtggcatgtacctgtaatcccagctactcaggaggct |
| | gaggcaggagtatcgcttgaacccgggaggcagaggttgcagtgagccaagatcgtgccactgcactccaacctgtg |
| | caacagagcgagactccatctcaaaaaaaaaaaaaattaaaaaaaagagagagagataagatttgagatctgacatg |
| | gagcttccctatttacactacttacctgctttgtgacctaaggcaagttacctcagctctccaatcactggttttgcaaggaat |
| | ttttttttttgtaaaatgttgtgaggattaaagatgtgtttttataaaagctacattttttgttgctttcttaaaatcagaagaattga |
| | attcgattttttttaaggtttctaatggaacttttacatattatttgttccagaacaaattccaaggaatggtctttgattttgtaac |
| | caaacaagtctttgatatcagcatcatgatcctcatctgccttaacatggtcaccatgatggtggaaaccgatgaccagag |
| | tcaagaaatgacaaacattctgtactggattaatctggtgtttattgttctgttcactggagaatgtgtgctgaaactgatctct |
| | cttcgttactactatttcactattggatggaatatttttgattttgtggtggtcattctctccattgtaggtaagaagaggtgcttt |
| | tattcagttaaggaatatagtggtaaaaatatgtgttttaaaactttagaggtgtttttcactaatctttctcattcatcccaaact |
| | cccaaataaaaatctaatagtccattgttttagttttagtttgccatttctctaattgcatgctgtgcttgaaatgatgagtggaa |
| | tacaaggaatttatattttcagctttcatttattctcatttaatattttcatctgttctcatctcagaagacaataactgcaactttg |
| | gtagaatagtcttgtacctggtcatactcctgtggtattgacagttactgctttgaataaacaatcaatccacacacatatata |
| | cataaatcatttgaagtagtcacataattcataaatatgacctcttaaataattggaatagtgtatatgtgcagttatatatata |
| | ataacacatatataagtttcatgttatctttgggtgcagacagttttctgtggtttgcaatatctctttttggaagcagatagttt |
| | gtttgaaaatccaaaacagatttgttatcatcaatgatacattaatgttaggatacatacatacattaagtcctaggaatgca |
| | aaagatttattggaaaaaatatatatatacagtgtttatgtataagatattaaatgaggtactggaagtaaatataagaagatt |
| | taagagaaggttctacctatttggggaaacagaacattcacatggaggggaaaattatatagcactctttaaactactttctt |
| | tagtcgaatagaacattgtaacaatgttccactgcatttgattctcacataagtgctatgaggtagcattaagaggtaagatt |
| | tgagatctgacctggagcttccctatttacactacttaccttctcagtgacctaagagaagttacctcagctctccaatctct |
| | ggttttgcaaggaatttttctgtaaaatgttattgtgaggattaaatcagattatgtatatatatgcacttagcactgtgcctag |
| | catgaagaaaagacttagtaaatgttcagtttgaccacaagaaaaagttgatattatcaccatttactcatgcataaaagca |
| | agtgccaggattcagtcccaagtacatctgtctccaaagcctatgttttcttctgtacatcacgctgcctactcccaaataac |
| | atagaatctcagaaagtaaagaactctcatattcctgacccaaaatcatacacctttagttcttatgcaaatactagaactag |
| | tattttggacatataaattaatttctgtacttggccactgtatgcttcatgatgtctttggaccttccagggttgagtcatttttttg |
| | atagatgctttccttgaactaggaaaaatggcccttattatcttcatttaatataaagatgtaaatgttataacaccaaacata |
| | ccagtttcattttgctcaacaaacattgcagattatttgcatatatacatgtacctaactgtcctgttcacattttgtaaaactaa |
| | tgtacttatgtaaactttcatttgctactattaagtataacaatatttttgttatttgttgattttctacaggaatgtttctggctgaa |
| | ctgatagaaaagtattttgtgtcccctaccctgttccgagtgatccgtcttgccaggattggccgaatcctacgtctgatca |
| | aaggagcaaaggggatccgcacgctgctctttgctttgatgatgtcccttcctgcgttgtttaacatcggcctccttcttttc |
| | ctggtcatgttcatctacgccatctttgggatgtccaattttgcctatgttaagagggaagttgggatcgatgacatgttcaa |
| | ctttgagacctttggcaacagcatgatctgcctgttccaaattacaacctctgctggctgggatggattgctagcacctatt |
| | cttaatagtggacctccagactgtgaccctgacaaagatcaccctggaagctcagttaaaggagactgtgggaacccat |
| | ctgttgggattttcttttttgtcagttacatcatcatatccttcctggttgtggtgaacatgtacatcgcggtcatcctggagaa |
| | cttcagtgttgctactgaagaaagtgcagagcctctgagtgaggatgactttgagatgttctatgaggtttgggagaagttt |
| | gatcccgatgcgacccagtttatagagtttgccaaactttctgattttgcagatgccctggatcctcctcttctcatagcaaa |
| | acccaacaaagtccagctcattgccatggatctgcccatggtgagtggtgaccggatccactgtcttgacatcttatttgct |
| | tttacaaagcgtgttttgggtgagagtggagagatggatgcccttcgaatacagatggaagagcgattcatggcatcaaa |
| | cccctccaaagtctcttatgagcccattacgaccacgttgaaacgcaaacaagaggaggtgtctgctattattatccaga |
| | gggcttacagacgctacctcttgaagcaaaaagttaaaaaggtatcaagtatatacaagaaagacaaaggcaaagaat |
| | gtgatggaacacccatcaaagaagatactctcattgataaactgaatgagaattcaactccagagaaaaccgatatgac |
| | gccttccaccacgtctccaccctcgtatgatagtgtgaccaaaccagaaaaagaaaaatttgaaaaagacaaatcagaa |
| | aaggaagacaaagggaaagatatcagggaaagtaaaaagtaaaaagaaaccaagaattttccattttgtgatcaattgtt |
| | tacagcccgtgatggtgatgtgtttgtgtcaacaggactcccacaggaggtctatgccaaactgactgtttttacaaatgta |
| | tacttaaggtcagtgcctataacaagacagagacctctggtcagcaaactggaactcagtaaactggagaaatagtatc |
| | gatgggaggtttctattttcacaaccagctgacactgctgaagagcagaggcgtaatggctactcagacgataggaacc |
| | aatttaaaggggggagggaagttaaatttttatgtaaattcaacatgtgacacttgataatagtaattgtcaccagtgtttatg |
| | ttttaactgccacacctgccatatttttacaaaacgtgtgctgtgaatttatcacttttctttttaattcacaggttgtttactattat |
| | atgtgactatttttgtaaatgggtttgtgtttggggagagggattaaagggagggaattctacatttctctattgtattgtataa |
| | ctggatatattttaaatggaggcatgctgcaattctcattcacacataaaaaaatcacatcacaaaagggaagagtttactt |
| | cttgtttcaggatgtttttagatttttgaggtgcttaaatagctattcgtatttttaaggtgtctcatccagaaaaaatttaatgtg |
| | cctgtaaatgttccatagaatcacaagcattaaagagttgttttatttttacataacccattaaatgtacatgtatatatgtatat |
| | atgtatatgtgcgtgtatatacatatatatgtatacacacatgcacacacagagatatacacataccattacattgtcattcac |
| | agtcccagcagcatgactatcacatttttgataagtgtcctttggcataaaataaaaatatcctatcagtcctttctaagaag |
| | cctgaattgaccaaaaaacatccccaccaccactttataaagttgattctgctttatcctgcagtattgtttagccatcttctg |
| | ctcttggtaaggttgacatagtatatgtcaatttaaaaaataaaagtctgctttgtaaatagtaattttacccagtggtgcatgt |
| | ttgagcaaacaaaaatgatgatttaagcacactacttattgcatcaaatatgtaccacagtaagtatagtttgcaagctttca |
| | acaggtaatatgatgtaattggttccattatagtttgaagctgtcactgctgcatgtttatcttgcctatgctgctgtatcttattc |
| | cttccactgttcagaagtctaatatgggaagccatatatcagtggtaaagtgaagcaaattgttctaccaagacctcattctt |
| | catgtcattaagcaataggttgcagcaaacaaggaagagcttcttgctttttattcttccaaccttaattgaacactcaatgat |
| | gaaaagcccgactgtacaaacatgttgcaagctgcttaaatctgtttaaaatatatggttagagttttctaagaaaatataaa |
| | tactgtaaaaagttcattttattttatttttcagccttttgtacgtaaaatgagaaattaaaagtatcttcaggtggatgtcacagt |
| | cactattgttagtttctgttcctagcacttttaaattgaagcacttcacaaaataagaagcaaggactaggatgcagtgtag |
| | gtttctgcttttttattagtactgtaaacttgcacacatttcaatgtgaaacaaatctcaaactgagttcaatgtttatttgctttca |
| | atagtaatgccttatcattgaaagaggcttaaagaaaaaaaaaatcagctgatactcttggcattgcttgaatccaatgtttc |
| | cacctagtctttttattcagtaatcatcagtcttttccaatgtttgtttacacagatagatcttattgacccatatggcactagaa |
| | ctgtatcagatataatatgggatcccagctttttttcctctcccacaaaaccaggtagtgaagttatattaccagttacagca |
| | aaatactttgtgtttcacaagcaacaataaatgtagattctttatactgaagctattgacttgtagtgtgttggtgaaatgcatg |
| | caggaaaatgctgttaccataaagaacggtaaaccacattacaatcaagccaaaagaataaaggtttcgcttttgtttttgt |
| | atttaattgttgtctttgtttctatctttgaaatgccatttaaaggtagatttctatcatgtaaaaataatctatctgaaaaacaaat |
| | gtaaagaacacacattaattactataattcatctttcaattttttcatggaatggaagttaattaagaagagtgtattggataac |
| | tactttaatattggccaaaaagctagatatggcatcaggtagactagtggaaagttacaaaaattaataaaaaattgactaa |
| | cattttaagttgtgcatcttttctccttcctgtccacctattgttctttttttcacttttccatttcaatttcttccttatgtattctt |
| | gatctacttttctttatatccttctatcctttccttgcgctctcagtatttttcatttaggatattctccttgtttcttttctgttcac |
| | caaatgtcttgtttattacagcctatagatcacttagatttagatccctaaaatttgctgtcactctgtaaagtgcacatagtatctgc |
| | gtatttatttttacaattttctttacaaaaattctaagaaagcttagactgcaacttttcataatatataaacaaaattccaaaatagca |
| | cagatttgactttctcatttaattatgaacagtttgggaaatgtttctcaattctgaatgcatattagaactacctgagaagcaa |
| | aaaaaaaaaaaaaaaaaaagccagtgcatgggccaagaccaggaccagaccaattgaactcatatctctgggtgaga |
| | cttaagcatttaattttttgaaggctcgtcattgatactgattcataccaaggactgagaactagcaaatttgggtctcctttg |
| | cccaataggaaaacatattctccggaaagtccaaactgtccttcattacataacttatcatgtacagaataaagttcctaag |
| | aggatggctttcggagtatttatattgcagattgagggattgacattgatctaattataatagaggttcttctttagggctgta |
| | gttggtataaagggaattctattagaaaagtttagggagactgtgttcaggtaacacatgcacacacacacaccccatat |
| | ggcaccttaactatccctagtgtatactgggtggaatgaatgtgggatggtctaggaaacagtataaatttataaaatagtt |
| | ccctccagtgaatgctacatgccctgttgcatgagaacctttaatctggaccaatagaaggcagttcatggtggccagtc |
| | agaaagccattcaatttgtcattaccaactcacaaatctagacaggaaagattctcattctcatcttctccattcttggcgcat |
| | ctcacacaatcccttgtttttctgagctctgtaacatacagacctatttacaaacagaagggaggagacaagagaggtaa |
| | ggggaagtaaagaaagaggagaaaggaggagagggaacgataggggaaagtaggagaaaggaggggagtgtaa |
| | gaaatgggatgtttcatgaattataattctgaaaaaatgtctggtgtggatacctccagcaatggtgagggaaggaggaa |
| | gaaatgttgcttagttgtcaatatgttagatctaaaatataaacatgtttcttctcttatttatttttatttgtatttatttatttatt |
| | tatttttgagataaggtcttgctctgtcacccaggctgaagagcagtggtgtgatcatggctcactgcagccttaaccttctggac |
| | tcaagtgatcttcccacctcagcctcccgagtatctgggactacaggcatgtgctcccacacccagctcattttttttttttttttt |
| | ttgtattttttgtagagacagggtctccccatgttgcctgggttggtctcagatttctggactgaagcaatcatcctgcctca |
| | gctttccaaagtgctagaattacaggcatgagccattgcacctgcccctctcatttttttaaagaaaagatacctgtgaaaa |
| | aagtgaagaggacattatacaagaaaatgttacaatgattccatggattcaggaatatttttttttcttttacctgaactaaata |
| | catatttgagtgtctactatatatcaactttgcaatagcagctaagagatcaaagaggaatataactatccaaagaaaggct |
| | ataaccagaggtgaagaacctggaaagccatgctagagtttggaattgattctgtggcaaaaaggagtgattactcctga |
| | gaagtagaaggaacagtgaaatatgacacatctgtagctgctgaatctggtagtaataaggctatgctgcccattttttcta |
| | gtattttacagcaatctcattttcccaattatttaaatgggttcttatctcttggtatagtatatagatttattaagtaaaagaatat |
| | aactagatttttaagtcatccaatgtgacacttcctgctaatagcctattttaatcaagttacattttttatatttacagatacatct |
| | aattttattcctatcactttacttcacactttccactttttgaggtttttccttttattctcctcttccttcttgtcctcttttgcttt |
| | gtgttttctgtttctctttcccccatctctgatggaaagttacacatcatctccctccattcatccctccctccctcctttccttcct |
| | ttcttcttttttctttctcttttataagtgattactcttaaattttaacatgcatacttaattaaggtgaactgtaatcaatatttgtg |
| | tcctgatctggaaaaatgaattttcctagactgatgacatatcattgctctcctctaatatgttatttttgtgtagtatcttaactcta |
| | ccttaatttgagataaaacaaattaaacagtattattacatgaatttgtaggcagtacttatttagacttacatactaattattg |

We claim:

1. A method for treating early onset developmental and epileptic encephalopathy, the method comprising:
administering a therapeutically effective amount of an oligomeric compound to a subject in need thereof, wherein the oligomeric compound comprises a modified oligonucleotide consisting of 12 to 30 linked nucleosides wherein the nucleobase sequence of the modified oligonucleotide is at least 90% complementary to an equal length portion of an SCN2A nucleic acid, and wherein the modified oligonucleotide comprises at least one modification selected from a modified sugar moiety and a modified internucleoside linkage, and wherein the oligomeric compound is administered intrathecally to the subject at an initial dosage of about 1 mg to about 100 mg.

2. The method of claim 1 wherein the initial dosage is about 1 mg to about 25 mg.

3. The method of claim 1 or 2, wherein the initial dosage of the oligomeric compound is titrated upward to a maximum dose during a dose escalation phase.

4. The method of claim 3, wherein, during the dose escalation phase, the dosage of the oligomeric compound is titrated from the initial dose of about 1 mg to the maximum dose of about 4-50 mg.

5. The method of claim 3, wherein, during the dose escalation phase, the dosage of the oligomeric compound is titrated from an initial dose of about 1 mg to the maximum dose of about 15-100 mg.

6. The method of any one of claims 1-5, wherein the maximum dose is reached after administration of up to about 4 to about 12 titrated doses of the oligomeric compound.

7. The method of claim 6, wherein administration of each dose of the oligomeric compound is separated by at least about 3 weeks, at least about 4 weeks, at least about 6 weeks, at least about 8 weeks, at least about 10 weeks or at least about 12 weeks.

8. The method of claim 7, wherein administration of each dose of the oligomeric compound is separated by at least about 6 weeks.

9. The method of any one of claims 1-8, further comprising a maintenance phase, wherein the maintenance phase comprises administering a maintenance dose.

10. The method of claim 9, wherein the maintenance dose is administered after the dose escalation phase.

11. The method of claim 9 or 10, wherein the maintenance dose is administered at least about every 12 weeks.

12. The method of any one of claims 9-11, wherein the maintenance dose is administered over a period of at least two years.

13. A method of treating early onset developmental and epileptic encephalopathy, the method comprising:
administering a therapeutically effective amount of an oligomeric compound to a subject in need thereof, wherein the oligomeric compound comprises a modified oligonucleotide consisting of 12 to 30 linked nucleosides wherein the nucleobase sequence of the modified oligonucleotide is at least 90% complementary to an equal length portion of an SCN2A nucleic acid, and wherein the modified oligonucleotide comprises at least one modification selected from a modified sugar moiety and a modified internucleoside linkage, and wherein the oligomeric compound is administered intrathecally to the subject.

14. The method of claim 13, wherein the oligomeric compound is administered intrathecally at a dosage of about 1 mg to about 100 mg.

15. The method of claim 14, wherein the dosage is about 8 mg to about 64 mg.

16. The method of any one of claims 13-15, wherein the oligomeric compound is administered intrathecally at least about every 12 weeks.

17. The method of claim 16, wherein the oligomeric compound is administered over a period of at least two years.

18. The method of any one of claims 1-17, wherein the method comprises inhibiting the expression of SCN2A in neuronal cells in the subject.

19. The method of any one of claims 1-18, wherein the subject has been diagnosed with early onset developmental and epileptic encephalopathy prior to three months, prior to two months, or prior to one month of age.

20. The method of any one of claims 1-19, further comprising determining that the subject carries a SCN2A mutation prior to administering the oligomeric compound or antisense oligonucleotide.

21. The method of claim 20, wherein the SCN2A mutation is A263V, E430A, E430G, R1882Q, G879R, G1593R, K1502N, V1601L, G211D, S1780I, D343H, R1626Q, G882E, M1545V, L210Q, Q1479H, N1662D, F1597L, V423L, A215T, I891T, Q1531K, L1563V, E1321K, Y1589C, M252V, R223E, L1330F, V208E, R36G, R1882G, D343G, V261L, F1651C, R1319Q, Q383E, V1325I, K908E, V261M, S987I, R1629H, M1338T, E999K, R856Q, S1336Y, N212D, E1211K, D195G, L1342P, R220Q, R853Q, R1435*, K503fs*, R937C, or combinations of the foregoing.

22. The method of any one of claims 1-21, wherein the subject is human.

23. The method of any one of claims 1-22, wherein the oligomeric compound is at least 90% complementary to an equal length portion of SEQ ID NO: 2 and is not more than 50% complementary to an equal length portion of SEQ ID NO: 1.

24. The method of any one of claims 1-22, wherein the oligomeric compound has a nucleobase sequence comprising at least 12, at least 13, at least 14, at least 15, at least 16, at least 17, at least 18, at least 19, or 20 contiguous nucleobases of any of the nucleobase sequences of SEQ ID NOs: 16-2531.

25. The method of any one of claims 1-22, wherein the oligomeric compound has a nucleobase sequence comprising at least 12, at least 13, at least 14, at least 15, at least 16, at least 17, or 18 contiguous nucleobases of any of the nucleobase sequences of SEQ ID NOs: 2532-2539.

26. The method of any one of claims 1-22, wherein the oligomeric compound has a nucleobase sequence comprising:
a) at least 8, at least 9, at least 10, at least 11, at least 12, at least 13, at least 14, at least 15, at least 16, at least 17, at least 18, at least 19, or at least 20 contiguous nucleobases complementary to an equal length portion of nucleobases 199863-199905, 227493-22755, 243124-243204, 247823-247921, 254142-254177, 168911-168945, 170026-170061, 183519-183562, 188630-188668, 199912-199962, 227419-227450, or 238173-238192 of SEQ ID NO: 2, provided that the modified oligonucleotide does not comprise more than six LNA nucleosides; or
b) at least 8, at least 9, at least 10, at least 11, at least 12, at least 13, at least 14, at least 15, at least 16, at least 17, at least 18, at least 19, or at least 20 contiguous nucleobases complementary to an equal length portion of nucleobases 243917-244073, 170174-170200, 176724-176751, 180772-180801, 183968-184016, 202877-202906, 224198-224217, 224199-224218, or 243918-243937 of SEQ ID NO: 2.

27. The method of any one of claims 1-22, wherein the oligomeric compound has a nucleobase sequence comprising:
a) at least 8, at least 9, at least 10, at least 11, at least 12, at least 13, at least 14, at least 15, at least 16, at least 17, at least 18, at least 19, or at least 20 contiguous nucleobases of a sequence selected from SEQ ID NOs: 336, 488, 2021, 2097, 2174, 2250, 2326, 2403, 2499, 2500, 2501, 2502, 2526; 181, 259, 643, 720, 796, 2504, 2505, 2506, 2507, 2508, 2509, 2510, 2511, 2512, 2513, 2514, 2521; 491, 567, 644, 721, 797, 2177, 2253, 2315, 2329, 2406, 2527; 29, 30, 107, 108, 185, 186, 263, 264, 341, 342, 419, 420, 1796, 1871, 1948, 2025, 2101, 2178, 2254, 2330, 2503, 2517, 2522; 1016, 1093, 1104, 1169, 1246, 1323, 1400, 1477, 1554, 1708, 1785, 1860, 1937, 2014, 1631, 2090, 2539; 18, 96, 485, 561, 638, 715, 791, 868, 2247, 2323, 2400; 174, 1328, 1405, 1482, 1559, 1636, 1713, 1790, 1865, 1942, 2019; 20, 98, 253, 332, 410, 1406, 1483, 1560, 1637, 1714, 1791, 1866, 1943; 21, 411, 1407, 1484, 1561, 1638, 1715; 24, 414, 871, 948, 1025, 1100; 25, 337, 415, 490, 566, 2099, 2176, 2252, 2328, 2405; and 182; provided that the modified oligonucleotide does not comprise more than six LNA nucleosides; or
b) at least 8, at least 9, at least 10, at least 11, at least 12, at least 13, at least 14, at least 15, at least 16, at least 17, at least 18, at least 19, or at least 20 contiguous nucleobases of a sequence selected from SEQ ID NOs: 1090, 1166, 2484, 2485, 2487, 2493, 2496, 2497, 2498, 2533, 2534, 2535, 2537, 302, 1513, 1667, 1744, 1819, 1896, 197, 148, 226, 1364, 1441, 1518, 1595, 1672, 1749, 227, 1292, 1369, 1446, 1523, 1600, 1677, 1754, 1829, 228, 1679, 1756, 1831, 1908, 1985, 2061, 2138, 2214, 2290, 1226, 1303, 1380, 1457, 1534, 1611; 2079, 2523, and 2477.

28. The method of any one of claims 1-22, wherein the oligomeric compound has a nucleobase sequence comprising at least 12, at least 13, at least 14, at least 15, at least 16, at least 17, at least 18, at least 19, or 20 contiguous nucleobases of any of SEQ ID NOs: 2487, 2493, 2510, or 2514.

29. The method of any one of claims 1-28, wherein the oligomeric compound has a nucleobase sequence consisting of 17-19 or 21-30 linked nucleosides.

30. The method of any of claims 1-28, wherein the oligomeric compound has a nucleobase sequence consisting of 16, 17, 18, 19, or 20 linked nucleosides.

31. The method of any one of claims 1-24 or 26-30, wherein the oligomeric compound consists of the nucleobase sequence of SEQ ID NO: 2510.

32. The method of any one of claims 1-31, wherein the oligomeric compound is an antisense oligonucleotide and comprises one or more modified sugar moieties, one or more modified internucleoside linkages, and one or more modified nucleobases.

33. The method of claim 32, wherein each of the one or more modified sugars is independently chosen from a bicyclic sugar, a 2′-O-methoxyethyl (2MOE) modified sugar, a 2′-O-methyl (2-OMe) modified sugar, a 2′-methoxy modified sugar, a 2′-Fluoro modified sugar, a 2′-O-alkyl modified sugar, a constrained ethyl (cEt) modified sugar, a locked sugar, or an unlocked sugar.

34. The method of claim 33, wherein the antisense oligonucleotide comprises one or more 2MOE modified sugars.

35. The method of any one of claims 32-34, wherein each of one or more modified internucleoside linkages is independently chosen from a phosphorothioate, a phosphorodithioate, a phosphoramidate, a phosphorodiamidate, a thiophosphoramidate, a thiophosphorodiamidate, a methyl phosphonate, a phosphoromorpholidate, or a phosphoropiperazidate.

36. The method of claim 35, wherein each modified internucleoside linkage is a phosphorothioate internucleoside linkage.

37. The method of any of claims 32-35, wherein at least 10, at least 11, at least 12, at least 13, at least 14, at least 15, at least 16, at least 17, or at least 18 internucleoside linkages of the antisense oligonucleotide are phosphorothioate internucleoside linkages.

38. The method of any one of claims 32-37, wherein each internucleoside linkage of the antisense oligonucleotide is independently selected from a phosphodiester or a phosphorothioate internucleoside linkage.

39. The method of claim 38, wherein the internucleoside linkage motif of the modified oligonucleotide is selected from soooossssssssssooss, sooooossssssssssoss, sooossssssssssoooss, soosssssssssoooss, soooosssssssssoss, and sooosssssssssooss,
wherein s=a phosphorothioate internucleoside linkage and o=a phosphodiester internucleoside linkage.

40. The method of any one of claims 32-39, wherein the one or more modified nucleobases is chosen from 5-methylcytosine, 5-hydroxymethyl cytosine, xanthine, hypoxanthine, 2-aminoadenine, 6-methyladenine, 6-methylguanine, 2-propyladenine, 2-propylguanine, 2-thiouracil, 2-thiothymine, 2-thiocytosine, 5-halouracil, 5-halocytosine, 5-propynyluracil, 5-propynylcytosine, 6-azouracil, 6-azocytosine, 6-azothymine, 5-uracil (pseudouracil), 4-thiouracil, 8-haloadenine, 8-aminoadenine, 8-thioladenine, 8-thioalkyladenine, 8-hydroxyladenine, 8-haloguanine, 8-aminoguanine, 8-thiolguanine, 8-thioalkylguanine, 8-hydroxylguanine, 5-bromouracil, 5-trifluoromethyluracil, 5-bromocytosine, 5-trifluoromethylcytosine, 7-methylguanine, 7-methyladenine, 2-fluoroadenine, 8-azaguanine, 8-azaadenine, 7-deazaguanine, 7-deazaadenine, 3-deazaguanine, or 3-deazaadenine.

41. The method of claim 40, wherein the modified nucleobase is a 5-methylcytosine.

42. The method of claim 41, wherein each cytosine in the antisense oligonucleotide is a 5-methylcytosine.

43. The method of any one of claims 32-42, wherein the antisense oligonucleotide comprises:
a gap segment consisting of linked deoxynucleosides; a 5′ wing segment consisting of linked nucleosides; and a 3′ wing segment consisting of linked nucleosides; wherein the gap segment is positioned immediately adjacent to and between the 5′ wing segment and the 3′ wing segment and wherein each nucleoside of each wing segment comprises a modified sugar.

44. The method of any one of claims 32-43, wherein the antisense oligonucleotide does not comprise a bicyclic sugar moiety.

45. The method of claim 32, wherein the antisense oligonucleotide is characterized according to the following chemical notation:
(SEQ ID NO: 2493)
Gₑₛ^{m}CₑₒAₑₒTₑₒAₑₒA_{ds}T_{ds}^{m}C_{ds}^{m}C_{ds}^{m}C_{ds}A_{ds}
T_{ds}T_{ds}A_{ds}T_{ds}Aₑₒ^{m}CₑAₑₛAₑₛAₑ,
wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

46. The method of claim 32, wherein the antisense oligonucleotide is characterized according to the following chemical notation:
(SEQ ID NO: 2514)
^{m}CₑₛAₑₒ^{m}CₑₒGₑₒAₑₒ^{m}CₑₒA_{ds}T_{ds}A_{ds}T_{ds}T_{ds}
T_{ds}T_{ds}T_{ds}^{m}C_{ds}T_{ds}Aₑₒ^{m}CₑₛAₑₛ^{m}Cₑ,
wherein:
A=an adcninc nuclcobasc,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

47. The method of claim 32, wherein the antisense oligonucleotide is characterized according to the following chemical notation:
(SEQ ID NO: 2510)
^{m}Cₑₛ^{m}CₑₒAₑₒ^{m}CₑₒGₑₒAₑₒ^{m}C_{ds}A_{ds}T_{ds}A_{ds}T_{ds}
T_{ds}T_{ds}T_{ds}T_{ds}^{m}C_{ds}TₑₒAₑₛ^{m}CₑₛAₑ,
wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

48. The method of claim 32, wherein the antisense oligonucleotide is characterized according to the following chemical notation:
(SEQ ID NO: 2487)
Tₑₛ^{m}CₑₒTₑₒGₑₒ^{m}CₑₒAₑₒT_{ds}G_{ds}T_{ds}A_{ds}A_{ds}
^{m}C_{ds}^{m}C_{ds}T_{ds}T_{ds}T_{ds}AₑₒTₑₛAₑₛ^{m}Cₑ,
wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

49. The method of claim 32, wherein the antisense oligonucleotide is characterized according to the following chemical notation:
(SEQ ID NO: 2493)
Gₑₛ^{m}CₑₒAₑₒTₑₒAₑₒAₑₒT_{ds}^{m}C_{ds}^{m}C_{ds}^{m}C_{ds}A_{ds}
T_{ds}T_{ds}A_{ds}T_{ds}A_{ds}^{m}CₑₒAₑₛAₑₛAₑ,
wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

50. The method of claim 32, wherein the antisense oligonucleotide is characterized according to the following chemical notation:
(SEQ ID NO: 2534)
GT^{m}CₑₛTₑₒGₑₒ^{m}CₑₒAₑₛT_{ds}G_{ds}T_{ds}A_{ds}A_{ds}
^{m}C_{ds}^{m}C_{ds}T_{ds}TₑₒTₑₒAₑₛTₑₛAₑ,
wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

51. The method of claim 32, wherein the antisense oligonucleotide has the following chemical structure: 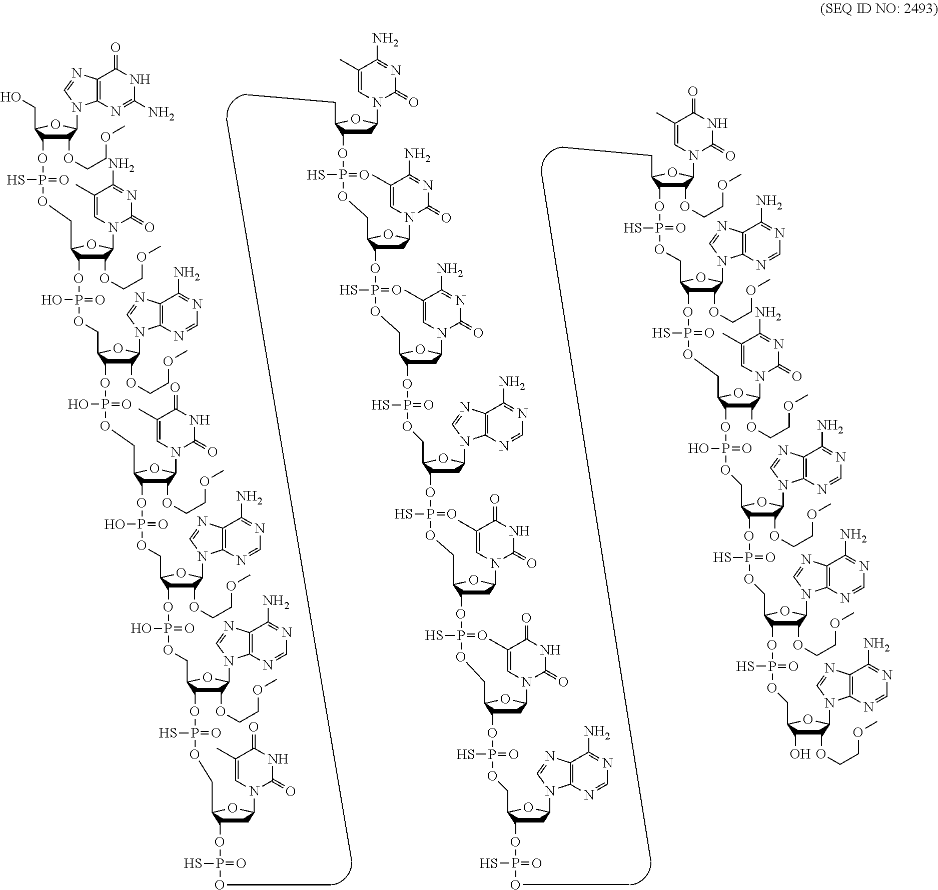
or a salt thereof.

52. The method of claim 32, wherein the antisense oligonucleotide has the following chemical structure: 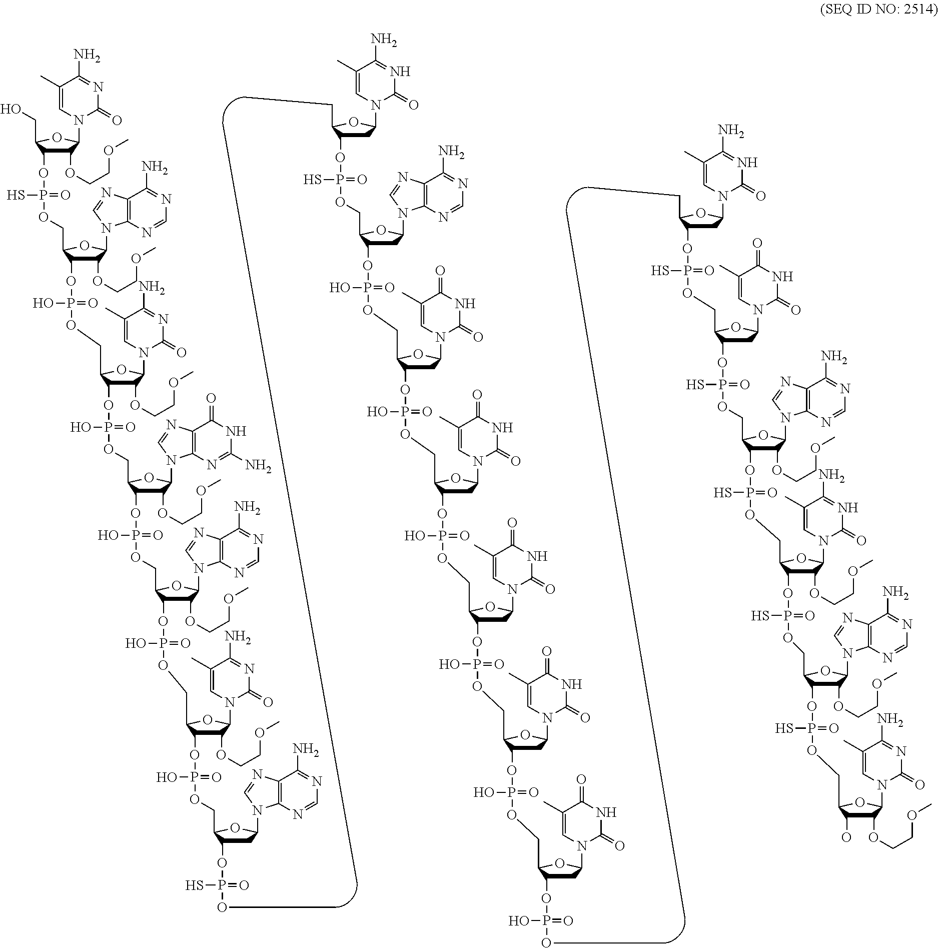
or a salt thereof.

53. The method of claim 32, wherein the antisense oligonucleotide has the following chemical structure: 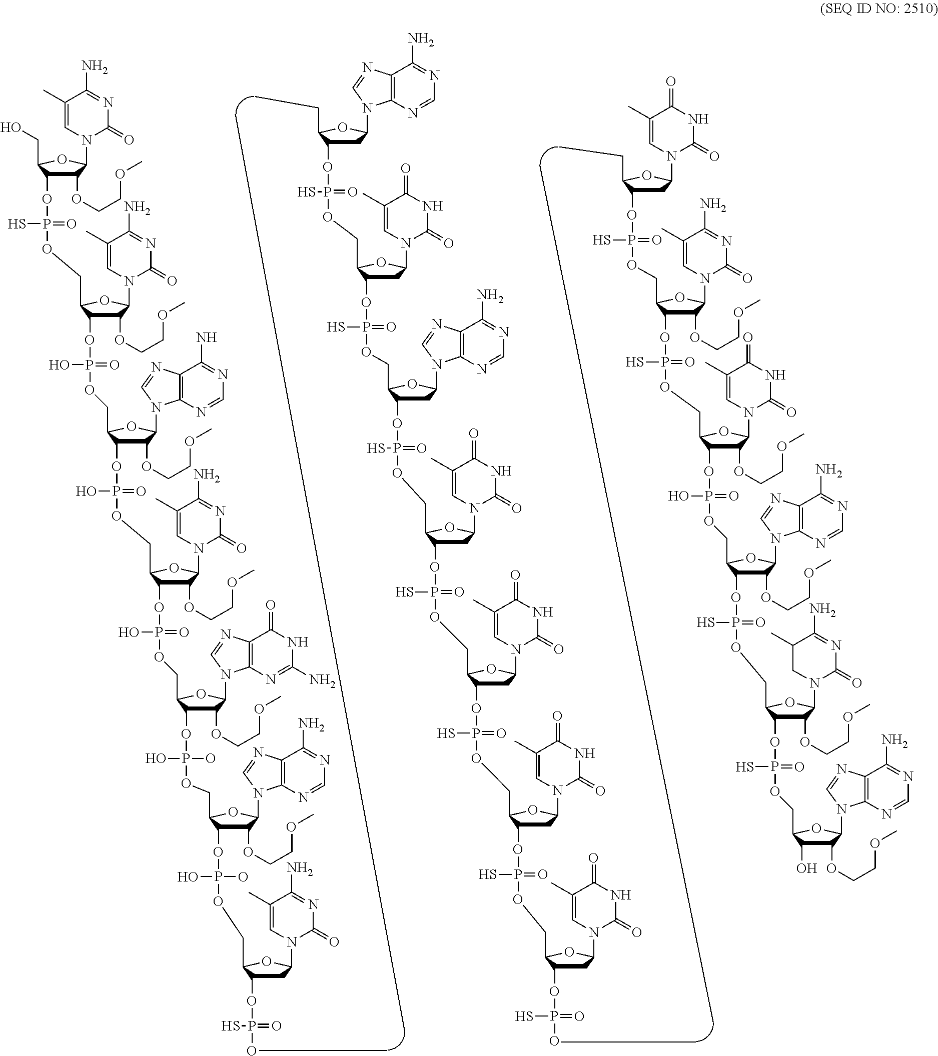
or a salt thereof.

54. The method of claim 32, wherein the antisense oligonucleotide has the following chemical structure: 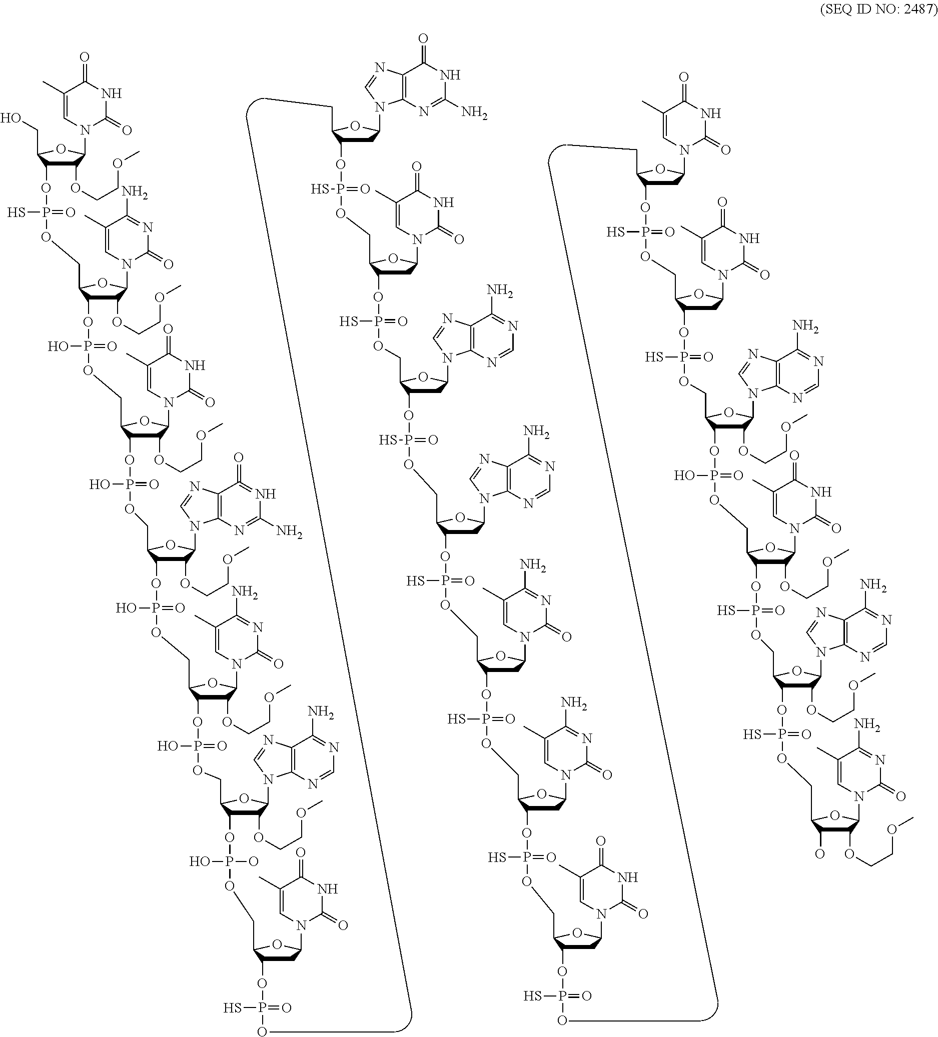
or a salt thereof.

55. The method of claim 32, wherein the antisense oligonucleotide has the following chemical structure: 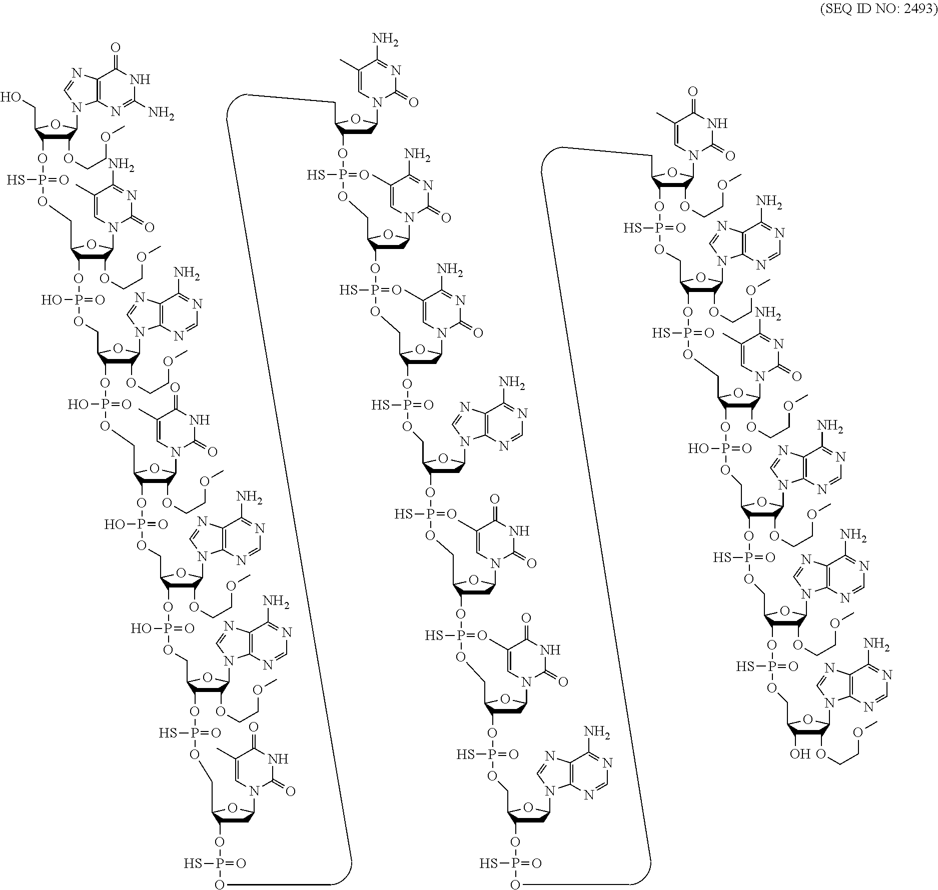
or a salt thereof.

56. The method of claim 32, wherein the antisense oligonucleotide has the following chemical structure: 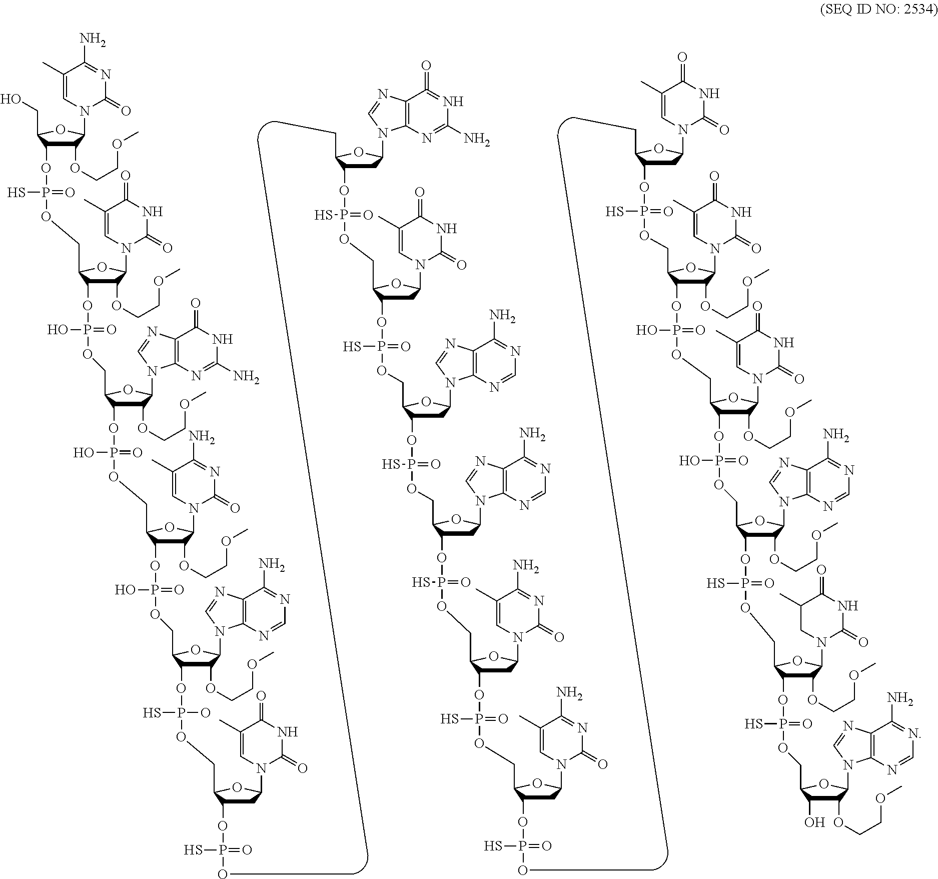
or a salt thereof.

57. The method of any one of claims 51-56, wherein the salt is the sodium salt or the potassium salt.

58. The method of any one of claims 1-57, wherein administering a therapeutically effective amount of an oligomeric compound results in a decrease in the frequency of seizures in the subject, as compared to the frequency of seizures in the subject prior to administering an oligomeric compound.

59. The method of claim 58, wherein the decrease is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75% or at least about 80%.

60. The method of claim 58 or 59, wherein the decrease in the frequency of seizures in the subject is observed at least 4 weeks, at least 6 weeks, at least 8 weeks, at least 10 weeks, at least 12 weeks, at least 14 weeks or at least 16 or more weeks after the start of administration of the oligomeric compound to the subject.

61. The method of any one of claims 1-57, wherein administering a therapeutically effective amount of an oligomeric compound results in a decrease in the frequency of interictal epileptiform discharges (IEDs) in the subject, as compared to the frequency of IEDs in the subject prior to administering an oligomeric compound.

62. The method of claim 61, wherein the decrease is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75% or at least about 80%.

63. The method of claim 61 or 62, wherein the decrease in the frequency of IEDs in the subject is observed at least 1 week, at least 2 weeks, at least 3 weeks, at least 4 weeks, at least 6 weeks, at least 8 weeks, at least 10 weeks, at least 12 weeks, at least 14 weeks or at least 16 or more weeks after the start of administration of the oligomeric compound to the subject.

64. A method for evaluating efficacy of therapy for early onset developmental and epileptic encephalopathy, the method comprising measuring frequency of interictal epileptiform discharges (IEDs) in a subject being administered the therapy.

65. The method of claim 64, further comprising comparing frequency of IEDs in the subject measured prior to administration of the therapy to the frequency of IEDs in the subject measured after the start of administration of the therapy;
wherein a decrease in the frequency of IEDs measured after the start of administration of the therapy is indicative that the therapy is effective; and
wherein lack of a decrease of an increase in the frequency of IEDs measured after the start of administration of the therapy is indicative that the therapy is not effective.

66. The method of claim 64 or 65, wherein the therapy comprises an oligomeric compound.

67. The method of claim 66, wherein the oligomeric compound comprises a modified oligonucleotide consisting of 12 to 30 linked nucleosides wherein the nucleobase sequence of the modified oligonucleotide is at least 90% complementary to an equal length portion of an SCN2A nucleic acid, and wherein the modified oligonucleotide comprises at least one modification selected from a modified sugar moiety and a modified internucleoside linkage.


SEQUENCE LISTING (2542 sequences)

SEQ ID NO: 1 - DNA, 8776 bp, Homo sapiens
  source 1..8776: mol_type = genomic DNA
aacagacattgggtaccatcgaatgactgtcagaacagaaagctaaggcaaaggagggaggatgctgtggtcatcctttcttgtttttttcttctttaatgaggatagagcacatgtgagattttactttctactccagtaaaaattctgaagaattgcattggagactgttatattcaacacatacgtggattctgtgttatgatttacatttttctttatttcagcactttcttatgcaaggagctaaacagtgattaaaggagcaggatgaaaagatggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaaacgtttatagtattgaataaagggaaagcaatctctcgattcagtgccacccctgccctttacattttaactcccttcaaccctattagaaaattagctattaagattttggtacattctttattcaatatgctcattatgtgcacgattcttaccaactgtgtatttatgaccatgagtaaccctccagactggacaaagaatgtggagtatacctttacaggaatttatacttttgaatcacttattaaaatacttgcaaggggcttttgtttagaagatttcacatttttacgggatccatggaattggttggatttcacagtcattacttttgcatatgtgacagagtttgtggacctgggcaatgtctcagcgttgagaacattcagagttctccgagcattgaaaacaatttcagtcattccaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtcacttttattttttagaggggcaaaatgatgctctgctttgtggcaacagctcagatgcaggccagtgtcctgaaggatacatctgtgtgaaggctggtagaaaccccaactatggctacacgagctttgacacctttagttgggcctttttgtccttatttcgtctcatgactcaagacttctgggaaaacctttatcaactgacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagagggcacaactactgaaacagaaataagaaagagacggtccagttcttatcatgtttccatggatttattggaagatcctacatcaaggcaaagagcaatgagtatagccagtattttgaccaacaccatggaagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctgctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagagaagctaaatgcaactagttcatctgaaggcagcacggttgatattggagctcccgccgagggagaacagcctgaggttgaacctgaggaatcccttgaacctgaagcctgttttacagaagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtctcactggttagcttaactgcaaatgccttgggttactcagaacttggtgccatcaaatccctcagaacactaagagctctgaggccactgagagctttgtcccggtttgaaggaatgagggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagccacgtttaagggatggatggatattatgtatgcagctgttgattcacgaaatgtagaattacaacccaagtatgaagacaacctgtacatgtatctttattttgtcatctttattatttttggttcattctttaccttgaatcttttcattggtgtcatcatagataacttcaaccaacagaaaaagaagtttggaggtcaagacatttttatgacagaagaacagaagaaatactacaatgcaatgaaaaaactgggttcaaagaaaccacaaaaacccatacctcgacctgctaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaaaaagaaaccaagaattttccattttgtgatcaattgtttacagcccgtgatggtgatgtgtttgtgtcaacaggactcccacaggaggtctatgccaaactgactgtttttacaaatgtatacttaaggtcagtgcctataacaagacagagacctctggtcagcaaactggaactcagtaaactggagaaatagtatcgatgggaggtttctattttcacaaccagctgacactgctgaagagcagaggcgtaatggctactcagacgataggaaccaatttaaaggggggagggaagttaaatttttatgtaaattcaacatgtgacacttgataatagtaattgtcaccagtgtttatgttttaactgccacacctgccatatttttacaaaacgtgtgctgtgaatttatcacttttctttttaattcacaggttgtttactattatatgtgactatttttgtaaatgggtttgtgtttggggagagggattaaagggagggaattctacatttctctattgtattgtataactggatatattttaaatggaggcatgctgcaattctcattcacacataaaaaaatcacatcacaaaagggaagagtttacttcttgtttcaggatgtttttagatttttgaggtgcttaaatagctattcgtatttttaaggtgtctcatccagaaaaaatttaatgtgcctgtaaatgttccatagaatcacaagcattaaagagttgttttatttttacataacccattaaatgtacatgtatatatgtatatatgtatatgtgcgtgtatatacatatatatgtatacacacatgcacacacagagatatacacataccattacattgtcattcacagtcccagcagcatgactatcacatttttgataagtgtcctttggcataaaataaaaatatcctatcagtcctttctaagaagcctgaattgaccaaaaaacatccccaccaccactttataaagttgattctgctttatcctgcagtattgtttagccatcttctgctcttggtaaggttgacatagtatatgtcaatttaaaaaataaaagtctgctttgtaaatagtaattttacccagtggtgcatgtttgagcaaacaaaaatgatgatttaagcacactacttattgcatcaaatatgtaccacagtaagtatagtttgcaagctttcaacaggtaatatgatgtaattggttccattatagtttgaagctgtcactgctgcatgtttatcttgcctatgctgctgtatcttattccttccactgttcagaagtctaatatgggaagccatatatcagtggtaaagtgaagcaaattgttctaccaagacctcattcttcatgtcattaagcaataggttgcagcaaacaaggaagagcttcttgctttttattcttccaaccttaattgaacactcaatgatgaaaagcccgactgtacaaacatgttgcaagctgcttaaatctgtttaaaatatatggttagagttttctaagaaaatataaatactgtaaaaagttcattttattttatttttcagccttttgtacgtaaaatgagaaattaaaagtatcttcaggtggatgtcacagtcactattgttagtttctgttcctagcacttttaaattgaagcacttcacaaaataagaagcaaggactaggatgcagtgtaggtttctgcttttttattagtactgtaaacttgcacacatttcaatgtgaaacaaatctcaaactgagttcaatgtttatttgctttcaatagtaatgccttatcattgaaagaggcttaaagaaaaaaaaaatcagctgatactcttggcattgcttgaatccaatgtttccacctagtctttttattcagtaatcatcagtcttttccaatgtttgtttacacagatagatcttattgacccatatggcactagaactgtatcagatataatatgggatcccagctttttttcctctcccacaaaaccaggtagtgaagttatattaccagttacagcaaaatactttgtgtttcacaagcaacaataaatgtagattctttatactgaagctattgacttgtagtgtgttggtgaaatgcatgcaggaaaatgctgttaccataaagaacggtaaaccacattacaatcaagccaaaagaataaaggtttcgcttttgtttttgtatttaattgttgtctttgtttctatctttgaaatgccatttaaaggtagatttctatcatgtaaaaataatctatctgaaaaacaaatgtaaagaacacacattaattactataattcatctttcaattttttcatggaatggaagttaattaagaagagtgtattggataactactttaatattggccaaaaagctagatatggcatcaggtagactagtggaaagttacaaaaattaataaaaaattgactaaca

SEQ ID NO: 2 - DNA, 268000 bp, Homo sapiens
  source 1..268000: mol_type = genomic DNA
ttttttaaagaacataatgtagttgagagtgtaatgtactaaataacatctcttttttggaaggctacttttttttgagatagggtcttgctttgtcactcagggttgagtagaatgatgtgatcgtggctcactgcagcctctacctactgggcttaagcaatcctcccacctcagcctcctttgtagctgggactacaggcatgtgccaccatgcccagtgaattttcgtatttttttttgtagagaggagattttgccatcttgcccaggctggcctcgaactcctgggctcaagtgatctgcctccttggcctcccaaagtgctgggattacaggcatgagccaccatgcccagcctaaaaggccacattttttaaccacattttcaaatgatcaagaaaatgaatatactaaagactcagaatggtttaatttcctgtgaaaaattggaatttttagaaatccatttcccaataaagagtatcttgtgattttccaacatcactatagacatatgactttcaatattgctttttgataatgcatataagcacaatgaaaatagtaaataactgtagtacatggttatcataggaaatggaacaaaaaatttaaaagcattcttacctctttgctttcttctagttctgactcactgctgaactcttcagtatttaagttttcaaagtcagactctccaacagcaattggcactgtgacggtgaggctggggttgtttatgaatgacatataatcattttcatcgattacgtatttttcaacactgcttccagtacctacaccactggtggttccattcccatctctaagataattaagctctttgcttatttcaattccagtattattggacatgcagctgtctatcttattgccttcatggatttctataacttttggctttctaaaaaaggctttttggaaacactcccgcatcttatttttcacataatcaattcccttttgcattcttcctactgcaatctgcagattattcatttcattgtcatcatcagtagcagcaaggttgtctgagctaaatgaactcaacaataaggccagaaagaggttcagaacctaaaagaaaaaaagaaaaatgtctattttaatattgaaatatgcatttaaataacagcctaaaaagggaactcatagatctttgctaaaattatttcaacaaatgtttattgaatgcttactatatttcagatgccatgtctaagtcacacactctttaaaggacctaatatcctgctctaagggcaaaatgataaaacaagcacctcttctttttttcctattgggagggacaataggaaagcagagcttctaagatttaaatggtgtctactcaggtttgccagtgcaaagacaaaatcctaccctcaaggattctgccaacgagagagagagagagagagagagaaagatactttcttcttaatcattttatcttacttcatcttaccattttttttcctagggaatttctagcttcgtgtatataaagaatgaacttatttatttaatagcctgatgatatgttccaaatgatatacttgaaatacaaattcctttggcccaatggccacaaacttttataacagcagaaatttctaagtgcaaattatatacgatttaacacgtttcccatcaaatacactttgaaatttatatgttcaaatagctcaacagatggagagaaatctaaaagtataccagggtttaggctagacccacataagttacttttctttttctttctttctttgtgtgtatgtgtgtgtgtgtgtgtagaaagtatgaggcgattttaactttcagaatatcatgggatatgtgaaaaagttactcagccaactgataagttatggttaatgcaggaataattcattatttctgatgtaaaagaaaatttataataatatttttagcctgaaatgtagttagttattgagtttcagaatatgttacactataggaagacatgcaaggcaaaagccccagtttgcttccttacttctgtatcagattttttcagaatctagagctccctaatttactattctggagaaaaaaattacatgttcgtaggtatcccagtagcattcttttgaacaagtttatctgaaatgtatccctcaaaggaaaggcccaatatgatggcataaagaaatgctttgcccacgcatgactgagaaatttaagcattaaacatataaatatataaactatgcttttgcattagtgattttaagtcacagttaaaagtaactcctttgagtctggacaatgtccatacatatgaataatttaaactgtaataattggttttcctatagtaatgcaaattcagggaagaaaacattttgtcctcttccaagttacttggataattacaatgcatagctaaaatataatggtctacttcattgaaatgtgtcttcaaaatttggtactatgcaacctatgtctctaagtaagcaaactacatgaactatgaacatgcaaagagcaatttgattatagaatagatagttgtctttttagatttagtagaggtcattttcttatcaaaacaatattaggatacaattcttcttagtgataaaaatttactttctttgaagaagaaaggaaaatctaatatttatgcccccaggcattcataaatacatctgatttaatctttgcattaaagatatgatttcagttttattattccaatgtcataaaaaatgggaaaccagagcccaggagaagttaaataacttgcttattctctgagtgctaatagggggatttcagtctaggccacttactataaaccagagatatgtttactacatctggccacgttcctagctactgtctgctcttgttctaatttatgagcatttgtactacatacataccacaaggtttccaatgaccatgaccaacatgaaaacaataaggcacatggtttggccagcgacctccatacagtcccacatggtctctatccactctccacacagcacgcggaacacaatcaggaaggagtggaagaagtcgttcatgtgccaccgtgggagcgtacagtcatcattgatcttgcagacacattctttgtagctcttaccaaagagctgcatgccgaccacagcaaaaatgaagacgatgatggccaacaccaaggtgaggtttcctagagcccccacagaattgccaatgatcttaattagcatatttagtgtgggccaggattttgccaacttgaaaactctaagctgtaatcaagtgaaaagatgctgttagtagtaatcataatataattttagacattattttattagtatgtggtatcatagaaccacacgtggtagatgtaaaaactcaaaatatactgaactgattcaatttcaaatccagaaaaaatagtgttaacaatattagaattgtaaatctggtcataatatcatttaagtgcaaaaataaaaataaaacatttcagaaataataaataaaacatttctgattcatccaaagatatgcccatgtaaacaaatggtattttgattagagactttttaaattagcaaataaatgttatcttcagattctattttagacagtaaaaagaaatattaaaagtataaatcatagatttttataggccaatataaaagtaaaataacctaaataatgtgataaaaatgtacaggaaaaaaattgaaaaaatacacattttgcttaattttcaataaattttacatatttctcaataaaattttgtgataattcaaaaagttttttatacaattatttaagatactaataacatcaaatattttcccacatgttgctttaaaacagtccccattcctagaagtagggttaataaaaagctaaaattaactggggtctaaatttttatttgcgtatctttacagactgtaaggccaaaaaagcttcataaacttaaaacacagttttagaaagtacttgtaggttttctaatgtgccctacatacctaatcattatagagtattttgaatgctttcatcattcttagaaaatggtttagctttaaagaggatgtttacatgttttcttagaatgtcacatattgatgtaaatttaaaattaaaattaataattatataaatatacattgattcaatttcaaaataaatgttgtgccaatgagcgacagggatatatataaatagataccagtctgaatgatcgcagtacagacaatccctccacatttgacagaccaagctccattaaactgaggctgacaataattccatcaaagatattccagccttcttggaaatagtaataaggatccatggcaatgatcttgagaaccatttctgctgtgaaaatcccagtaaagacctaaaaaatagagatcagcactacttcaagagcactgaaaaacactgatgctacacgaatttataaagaagaacattaacaggaataaaaatatctaaaatttaaagtgagagaatgtgacaaatcaattcaagtcttagtttttttattttcattttttatttttatttatttttatttattttattttattattattatactttaagtttgagggtacatgtgtacaatgtgcaggttagttacatatgtatacctgtgccatgctggtgtgctgcacccattaactcgtcatttagcattaggtatatctcctaatgctatccctcccccctcccccgaccccacaacagtccccagagtgtgatgttccccttcctgtgtccatgtgttctcattattcaattcccacttatgagtgagaacatgtggtgtttggttttttgtctttgtgatagtttactgagaatgatgatttccaatttcatccatgttcctccaaaggacatgaactcatcattttttatggctgcatagtattccatggtgtatatgtgccacattttcttaatccagtctatcattgttggacatttgggttggttccaagtaattgcttcaaagagaataaaatacctaggagtccaacttacaagggacgtgaaggacctcttcaaggagaactacaaaccactgctcaatgaaataaaagaggatacaaacaaatggaagaacattcgatgttcatgggtaggaagaatcaatatcgtgaaaatggccatactgcccaaggtaatttatagattcaatggcatccccatcaagctaccaatgactttcttcacagaattggaaaaaactactttaaagttcatatggaaccaaaaaagagcctgcatcgcccagtcaatcctaagccaaaagaacaaagctggaggcatcacactacctgacttcaaactatactacaaggctacagtaaccaaaacagcatggtacttgtaccaaaacagagatatagatcaatggaacagaacagagccctcagaaataatgccacgtatctacaactgtctaatctttgacaaatctgagaaaaacaagcaatgggaaaaggattccctatttaataaatggtgctgggaaaactggctagccatatgtagaaagctgaaactggatcccttccttacaccttatacaaaaattaattcaagatggattaaagacttaaaagttagacctaaagccataaaaaccctagaagaaaacctaggcattaccattcaggacataggcatgggcaaggacttcacgtctaaaacaccaaaagcaatggcgacaaaagccaaaattgacaaatgggatctaattaaactaaagagcttctgcacagcaaaagatactaccgtcagagtgaacaggcaacctacaaaatgggaggaaattttcgcaacctactcatctgacaaagggctaatatccagaatctacaatgaactcaaacaaatttacaagaaaaaaacaaacaaccccatcaacaagtgggtgaaagacatgaacagacacttctcaaaagaagacatttatgcagccaaaaaacacatgaaaaaatgctcaccatcactggccatcagagaaatgcaaatcaaaaccacaatgagataccatctcacaccagttagaatggcaatcattaaaaagttaggaaacaacaggtgctggagaggatgtggagaaataggaacacttttacactgtttgtgggactgtaaactagttcaaccaatgtggaagtcagtgtggcgattcctcagggatctagaactagaaataccatttgacccagccatcccattactgggtatatacccaaaggactataaatcatgctgctataaagacacatgcacacgtatgtttattttcattttttatttttattatttttttttgagacagagtctccctctgttgcccagattggagtagagtggcgcgatcttggctcactgcttcaagtgattctccagtctcagcctcccaggtagctgggattacaggcatgggctacctaccatgcccagctaatttttctaatttctgtagagacagggtttcacatcacgttggccaggccagtttcgaactcctgacctcaagtgatctgcccgcctcagcctcccaaagtactgggattacaagcatgagccaacacacccagccatgtctagcttttttttttttttcctctaggagagtattttattaagtagagtggtattatttaatgaatcttctggatttccaagaaaaacactaattttaaaacatttcatggtctacattttaactctggtattgaataatagctactgaattattgcccttgttggtacactacatttctaacataatcacaaccaaatttatttctgggtagtatcgcatcaggaacaaatccacatattactgaaagttattaaaaattgatttcgtaaaaaccatattttgtttttgattttgtgttttttaagggacaccatcttatttctataattttataaacattcctgaaacaaaagtaagtttgccacaaattgtttattataatgatctctggatgtatgcctcagaattaaggttaatggattaatagagcaagaagctgcagatttaattgaaaccagcaggagaaaatactcagtatgcatatacatgtaattgagattaataagtcacttgagtggctagataaatgtgtatttgacaatagaaagaaaaacaatgaaacaaacacaacatttattttttaactcaaaacgataggctgagagatacaggacatgaagaattatgaaattactcttgcagaaaatcttttaaagcaattctcttaatttgctatagttacaagttgcagtttatgactgtaaagtcaagtttagcatttacgaagataaatcagcctttatcttgaaggaattctgtcaaaataacagtataacaattatttggttccacttaaaagaaatcttgatccacccttacgaatatataaatgcagctaacacttcattttttccaatcacagatgggaaatacccatcacagaaaaaaacagcctaatggtagtcatccattcactcaaatagaaataaaatgtattttaagcaagcctagtacatgaaaatccaagtatatggtgaaatgcccattgaaaaatatatttttcattttatagaataacacatcaaagatttttttttcaaatagtgagtttgcctggtaaatgtaaaataagttgtaactatacaaattttatttcatgtataaaacttttagaagcagctattatttgaaataggcactctgatgatagaaatatattatttaaggtgagctagaaccatccaggtaaataatctacaaatatctctttctatagaaaatcataaggtgatcttttcagctcttctaaaatgactagatctcatacctttaaaaacatataccctaaatttacgcttatctaaaaatgtaatcccgccataaaacttttcattaaaaataatatttttattacaactagcaatccaaaatgacagctatatctctgtaactgttttcagttaaggccctaaaaatcaatgggattgtttttggtacattttaatctagtttcagcattgtcaggtaagttggaaatgtttgttatgatgttgtctaacctactatgattgataggctattttttgctgattgaatccctatgatgggagcaagcattttataatgattaaaaaacaaaacaaaacaaaatactccacaaaaatacggagcaccttagcaaaacttaattgagattcaaggtctcttgatactcaaatttgattaatctcatcaacaaggaagtgatgtaatttttatgtactgcagaaacagaatgcaaaatggggcatattgatctaagttaagatggaaaattccacaattcagtgtcctcatgtagaacataaagcagtatgcaataatttgagcagtaataaactagtctgtagatattctaaatttattttaatgagcaaattataagctctccacaaaacattctgtgtaatcatctatgatctataatatagtcctaagtcagcttagcaccaatgatagaaattctttctttctctctcacatgctctttctctgcatatatattaaacatatgtattactttcatatggatatttatgataaaggttgttatgggtaggaaattctatgggtagatattctatgaacaaatgctttgtaaattatgcaatgcattattgatatgttaatcattatagtaaggaggtatcaaccaatagagacagtcagtttacatattctcctatttttatggatagaaataaataactcaagtctgttccctataatttatgctctctatcttggcaagttttgtttccccaacactaacctgaacttatagatggttctatcacaaacaatctaccaggcatcagtctcccattccactttcccacctcattatttcagttactacccataaagtcatagaattgtagaactgggacatacataaaaagtcatttagtccaacacactcattgtttatatgtgtgttgagaaaatagactcaaggatctaagtgacttttcaaaaccacacagatatttaccatgaacactttgactaggacctagatttgatgcttttgtctagtgctttttcattcaatcatcttataatccagcctctcttatcctgtgtttccatactcaactgcttcagcattggaattctatatttcctgcttcactctggcaaatttccttcacctgttcaacttccctaactgtccaatatcctgggaaaaatacaagaactacaccagaaatattttccatcattatttcaaagaaaggtctaaaaggttaatagcatatatatgaggaaggaaactgtttattgagcacatgctagatgccaggcacagtctaggtgctttcacattacctcatgtgaccttcacaacaactctgcacgaagggtattattacactcatttcaataatagaaaacaaagattcagattgtttatcagaattgtccagaggccatatcactagtaagcactaggcaactaaggtcaaactcaattccactccactgcaaagtctgtgctcttttgcaaaatcatgctacttgaagcagcctttttgggtagttatcaaccagtgggttatttattgagtgactagcatgtaaacactgagagaggcaacagaaaaatgggacacggcctttgccaaagatgaagaaaatctcaagaatatggtgggtgttctactgagtcaaataatgcagataatttgaggtcacttattagggcacacaattatccaattgtccaccagcatttcctttcccccttctgttggcaattgctccttcacgcatgattcctcaggactgccatgttttcaacatgatcccaccctctgccaaggctgattaaatcagaggtgagcatgtgacctaaattatgcctaagttctaccaggagagtctttggaatgagactcaaaaggagattcatgcttttcagtgtagaggttgtaggatggaaaatcagtgagctcttgctgaacatttgctgacctaaagacaaatgatgcagtgaacttgtgggccatatctaagcccttgatgaaaactgttcctaaagtcagactgcaatactgccctttacttagtttgttagtctaccttttccctccattccagtttccccactaccccttgccccaagtttgttttagtacagagaaagctcttgattaataatatatgtaaacccttttcttacttgttcacccattttgattagcacacatagacacagatgaatgctcagtaatttgaaccagaggtatggctaaatcacaaatgcaaatttagcaactattttttttcttgtatgattatcactgaagctttttattaggtagttaacttgaaacaatgatgttctttttttcatatacatcaatctttgggtcatcccttcaatggacatgattgttttagaggctgtgtgggttaaaaccacatgattgtggagccagtattagatctcctggtttatctggacccacatttccagcaagaacaactgtaaagattttgagacgtttacaactggtaagacaccatgatcaatcagcacagacattggcctaaacaatcagcattcataccagttgagttttaggctgtatctctgagattccctgtaatattccttagatatttctttctatgaggaaagaggatataatttttgggttcaacacagcacaaatacatctttccctttggtctatctctcccacatctaccaaagtaaataagtaaacttcaaatgtacttaccaggtttcctacagtcaacacactactgaattgctcagtcatggggtagtgctccatggccataaagagggtatttaagacaatgcaaatagtgatggcaagatcaacaaatggatccataacaattaaattcacaagatgttttacttttaaccatgcatcacagcagtcccagatcaagaacacattggcaaatctataccagcatggcggacatttctgtctagattcttcaagttctggagggacaataacaaggaagagtagtaaataacaacaaaaatgagttattattgctagtagattttacattatttaacaaaattgatgcttatatttgaaaatagaagttcatattacttaagaaacttgagaagacaatcccattaaatattttcagaaaatgttaattaaaatgtcatatagatcgacttttttctcttttgaatacagaaaataagcactttgaatttaatattttgcttttaaaatgactaggttatgattgattaaagtgctgtattctctttgtcaaaacagaattttaaataaacattacacaactattaaaattttaaataattattgtttcttctgaaagtacctttttaaaaacattgtctaaagttgtgatcccttatgtgtgtgataacatttatgaactattctctcctctcatagtttccatatcaacattaatcttgcacacagtgagaacgtagtatgcctttctttttagtataaattcagactcaaatctacttttgtggtttgtacatatcttttagaattttagcatcatgtatttaaaatattgcttgaaattttaatggatgaggcctgttacaaaataattattatgtgactgtatatctaatacaccaagaagacatgatataaaatgtaattgatgttttctaaagaacatatgttagaattgacatataattgatctaaaaagggcagtactaatatattctctctccagacttcaaggagtgagggctgtgtgatgtatgaacaagtttagtgtataaatagttgctgcctattcctctgtgtcaaaatcaacaaacatttctttccgtggtcctttggaggaatgtaatggttgctgtgtatgtagaagatggaagagagacccttttgtaggatctctcttgcagtggaggacaggttttgatttgcccaactcatttaagtgctcccgcaaccccatcaacacttccatgcctgaagaacagactaaagcctgaaagaaatgtttccttatgcttctatataccaagacttagaaaaggggaagagtttactctatttgcatatgagtaacataacataagaataaatcctcattgatataatatcaagtgttgaaatatcactctgcaagtaaggtaaggacccaagtcaaaaagaaataaggccaatattagtttttgtaatcaatgtgataatgtactccatatatcacattcttaggtctaatatatagtttcgatctgggtaacagacttcagtaattctatgaaaagaaggaatagttcacaaagcacataatcacagaaagttggctgttccatgacctgcttcttaccttccattgtgttggtcagaatgctggctatgctcacggctctttgccttccagaggaatcctccagcatctccattgaaatctggtaagagcttaaccttctctttctgacttccgtttctgtggtggtgccctgcaaaccaaatactgatggctcaaaccactcttcccaataagtaatacaacaccacatagcatttctttggcaaatcatgctacatgcaatttttccaggtaagaattttcaaggaataaaatattgaattattgctaagttataaacagttttttttttaaaaaaaagttatcacaggtagtactctttaagaggaattcaccagtgggatgcagttttataacacaattatattacagaactattttaattaacctgtcacaattttcaaggaaatttgttgtaaacatgctaaagtcatttattattttatttcattcaggtgaaaattatttttatcttctatcaatgattaataatcagatcaatctccccccaataaatattatgattatatagtctcaaggctgaattattttctttgaggctatattgacaatattgacatatgtgggccaattttttccatgcacaaacacagatgtacgatgaaatgtgtgtgctttcctgaaatttctttaattatttctatatcttatcctattaaaaaacaacacattttagagtcattttaaaatagcactgtaaatattgacagttgtatgaaattgcccattatcaacaaattggttataatatttaattttaagaatatgaattttccagaatatgaatagataattatacaattcttggtgaatatacatcttatagatttgatggtgaaaatgtccattatttgtctcggtaggtctaccaaatttgcagatatgtatttcatagcagactctaaaaacggggcccttttaacctaaagccatggtcccttacaagtaattcccatacaacaaaagctaatggtcccatacaattcaattgattctcaatattctattgttttccctttgattaatcatgtattatttttaccaactttcattttgaggaagcaggacggtatgacagcctaaactgtccaggctttgattatttcaaattggtgaataatgtcagtagcagctaggtcatctattatcacctctgggggaagttgtccagtaggtgacgttagagctgaaggtccacccaccaaggaaaccacaccattgcaatccacagtgctgtgcatcttcccatttgctggaagccctggcaccatcctggatgacatactggcctgactaacgttactgttgcgtcgctctccatgtctgtgcggcacaaacagtgagtctctcctgctttcgctgtcttcaaatgtgctgtgttcatcatcagcaaagtcattttcagatccaacatcctttgcccgacctctgaaactgaaaatgcttgttttgctattgcgtcttggggaaaacagggagccacggatactcaagagagactgcagagaaagcaaaaaggaaaggaatgggatgggggtaggggaagaacgcaattatttaataaatattaatattaaaggatctgttttctttggagttagtatactcttgctcttaaacatggataatcaaatttatagaggacacatatattttgatatttttattaaagcaaatttaagtacaaagagtgtttttctgactaacaatactgatttgcaatttactaagtatacaaccacttcatttagtcttcactttaggtggattgggttatcataatgctataggttagggaccagatgctgaaaaggataaatgatttcttttgatgggaatggtggttaaacaaatagtaagtagcttctacttcttaatgctataatctgaagggaatgtagcctggagaataagttttggctcaagtgagaatgttagctcaaatttaaaatatttgtttcaaatgttgcattgtagccaatatttcacttcaggataattacttttttaaaaaactactatttttccattctcttcttttgaattctgaaagaaaagtagtttactctttctctgaaactgtttgtctctacccacatgggaacagaagtcacaaaacttacctacgatgttgtcttccaaatttgctaacagtttcccaaatggcaaccaatgattcaccttatttgagagactatatgtcatagcaaacacaacacagaggattcctaaactgctacacaaattactcttcttgtgtaaaatataacactggcttgtaaaagcaagacatataacatgacttcaccagcaggtggtgtgaaaaacaacatatacttctgttttattttaattatattaaaaagaaagagacaattcaaaacctttttcattacagctattcccagataaattaagcaaactatttcctagtaccagaaaatgaggatttagcttgaagttccttctttcacattttgaaaggattttaaactagcaaccccaaataattctgtgtgtattcttgtcttgcagatagttttcaaaaacatctctattcagtctgcagaatatttgttttctcccctcatcatgaacaatggcattcctccttgctctctcattccctttcctacttctcttcacacccagggtaatatacatacatacatatacatatacatatgtcagcagcaaacattcctggatcttctctttcttctaagctttagtgtaaaaccaaataaaatgattttagtggaaaaaacagaaaaggcagactctgtcaaatgtattcttggttcctctgcctaattaggtcatgctgattttcttaggccccaatttttagctttattttcatggcttttcccagactgcaaagcaccagaataatctttgttgtcctctgtctcagtgatattctggaaggatattattcactaagaagtgctctgttcctttggatttggttagattctccacaggaactattggctgtccagcttgtccatcggaggctgtgtctatgtgacactcaattcctgtagcaggtttgtctaccgaagggcagacaatagagtgattttacagcaaagtgatcaagagattagatcatgtgaagacatcaattcatccccccattaataaagaatgctaatgtttttcatttcctcagactccatttaaatgatcaatataataacagttatatccagaactcgtgttgttgttgttgttgttgttgttgttgttgttgttgttgttttgaggcagagtttcgctctgtcgcccaggctggagtgcagtggtacaatctcggctcactgcaatctccacctcccaagttcaagctattctcctgccttaccctcctgagttggctgggactagaggtgcccgccaccacacctggctaagttttgcatttttaatagagacagggtttcaccatgatggtcaagctggcctcgaactcccaacctcaggtgatctgcccgccctggcctcccaaagccctgagattacaggcatgagccaccatgcccagcccagaactagtgttttgaaacatttgagtgtatgtgaatcactctgagagttaaaaatatatatttttaggtcccagattttggaatatggattcaggaagtagcagataggactctggaatctgaattttcctgacacattccaggtgaccctaacacagggtggacctcatcctacactcagagaaatactaacttggtcttacattaatccctaggatggctgagtataatcacctcaatacacctatgttctgaaactgtctctggaaactgaaaatcggtctacacagtattggttgggagtatgtggtacgtgtgtacatccactatgtatctgggacatgcctttaatttggatatttttatgtcaggtattttcaaaggcgttccagatctctgagtttatttgaaatttctattcactctttggaatgtataataatgggtgtgagcgcagctttcaaggcaacagagacggaaaaagaagataacagaaatagcattcagagaaaataaaccaaaaataacacactagaagatgcagcagagtggagggaatacccacagctgtggcaacactttctctagggaaaaccagctttaatgttttaaggagtgaaaggccatctttctttacctcaggcatgtcaaaacatatttcccaggtatctttttcatcttgttcccagaagataaatgtagactgtttcttaattttcagtaacaaaagggaaaatagaagaaattaggtctataagacacagtcatgcatgtagggtttggattcaaagtgggttgggaaaagtcaagatggaaaaagatgctgggaattttaagatgttctagtgggaacaaggaaagtgaatatttgtcagaagtacttagcattttgaggtttcattttatacttttaagtatttctttattttgtacaactaacattatctgtttaaaaataaaataatgtaataaacataaaataacatttgtaatttatgtgaatagaaggcagggcacccacaatttatgccctctctgctttgacattttcagaagatacatttgatttgagcagtatttactgtctatcttacgcttcctgtccccaccaccttcactgattctctttctaccagattccttttttgtttacagaagcagaaattgtaaaccgtttatggtacatttctgcttgttagttttgttaatgctcttagtctgtctttactgggatatgcaaatttaaaactaatagattatttccttcacttttgaaattatccatctatcttctgtctatttacaaatataaaatgagcgacatctaccacatgtatgtactgattctgcttttagtattaaaacactggccatcagctaagctctctgctcttgcctgtctgtggttcactcagctccagcaaaccaagtctaccagttgcttcttaagcactccaggcatgctaccatgtcagggttttgcacttcctctttcatcagtttagaatgctgttccttaagagagcctctttctttgcctcctccaaatctttcccaaaatgtcttaatagtgaggtcattgtaaaccaccctatttaaaattttacctccaattatggctcctgttcctcttttcctgcttacttttttttcccttaacatttgttaccttctaacacagtctataacttatttattttattcattgtccgtctcctttggaaaaaatttccatgaggtcagtggattgagtctcttttattctttgaggtattttcagcatcttgaactatgcttgctacatagttggcgatcaataaatatttgttgattggactgaatgaactgatctacaaaatttatttgagcaaaatgatgaaacaataaaggaactaatactgctatcaaattatgtatatacatttgttagtaattgaaactactcagaagtttgctttccctcacttgaaattcattcaccgattttaacttaataagagacttactctttgtcacacagattttctatatattgcttagtcccctaaatctctttaatgtaggtattcttactttattttacagaaattttacaaagtacaagtgacttgaccaacgtcacagaactagtttggagttgagatatgaacacaaaagtttgtgttcactgtagcatcattatgctatagtttcatatactcttaattctaataactcctaatagttttaagaattccaaaatatcaaaggtgactgtttaagaaaaaaaaatctgatctgaggaaccaagttgcccttcagagcttcaagttaattccaaatatgttatactgatttgataatatattttatatttgtattacacaagatagtaatagttgaatttcttgatattgatgttatctgcagaaaaatggctttatcttaaaactctttgtgtattcatgtccattttgttctaatatgagaatacaagttttaagttattaatatagtctacttttttctttaaaaatatttgtttacatattaaagatagattataaaacattcactgagaacttgcatattaccttttctcccaaccaaaagtaaatactatgttgaattttgtgtttattacttctggttttttttagaagtttctttctttatgaaaaatatatcataccccttggttgatttgcagtttttgattaaattaatcttatatattatctctcaaagcattatttttagggtataattctgactcaagattgttatgattagaagcatttgcaattcatagcacatgaaattctaatggtgccaaatagtgttcaaagccaaaattgtacttatcacagattttgtaactaattattctttacatcagacatatgtaaacaatacattaacttactaatccataaaacttactaaacatcaaataattgaaactagtttttactcttttacttaaaactatgctgaagaaccaggtagttggcattaaatcagagcaaaaatagactacctttgatgctgttctcttcaagaacatttaggtcataatttcattctaatgtatcacggcatgaccctatgcaagcaaaatggagactcaccatagtccagttctcttaatcaaaggataaagacaaaacccaaagaacacttcttttcaaaatttatatgtatgttacgatcacaactttggataacaaaggcagaaacccattctcaaagaaaataagtttatctggggtctcaaaatggaaaatgtaaacaagagattccaaaactctcattgaaaagtctgctagtgtaggttatatatatatatatgtgtgtgtgtgtgtgtgtgtgtgtgtgtgtgtgtgtatagacacatatatacacacatatatatatacaggttgatatataggttatatcaatatatattatatatatctatatattatatcaatatatctatctgtatatataatcagtctcaaaatggtgaacattttattaatggtttaataaaaatccctttgaagacaatctattataagaatttaaacaactatattaaatgttagttccctgtccctcactgaagtcagatagtacaaaagtgtaccaaaacaaaatacaaaaactaaaaagcaatagcaatgacaaagaaaccagagcacttagtagaaaatcatacctgatgaggggagcagaattttttgtcactggtcagtctgtttccatccatggagaaaaggaagctgcttcttttgacgctgtcttcagattcggatttgggaaagctgtctctctctcctttgttgtttccttcaaggtgctctctctgtcttcttttcttccttcggttcctccattctttagcacttttggaactcaactttgatgcttctgaagaactttccaacagctctcctaacccacctattccactgaaatctcttgaagcagctgatgctgccgcaactgcctgtcataaaacaaagccaggcactatttagaacacagagctttgaaaacagttgagtatggttaaaatattgccatttaagactcattaactacaagtgaaaagtctatcctctagtccttttattttgtctcaccacaagagtttacttaacaatgacccttatgttttaaatcatataaagcaatatgctagtgtatgcattaaagcaagaaaaatctggttttgcctatcttctggattgtcttttttttggggggggggggttggtgacaactgttctcttcaaagtgtcttctatactttaaaaagcaaatgttttcttcgtggtggttgcttatctaactaccattctgattccatctgtaaaacgtttcctcctgtgtgcacttctgagcagctgagtcgtattttttcccctctaaatccagtacaaaagactgtttcttttattttttgtgaacataaaacatttcaagtgtctactattttacctccagatggaagtagggaaatgtattagatagcttatcctcttccttcctttgaattacatttaggactgtagttcacatctctgtgatcatgttactcccagagaatttattacatatcacttaaaaaatgttaaacgttcatagattatttaatattataaataacaaatgattaacatagcattacataaattcattcaaaggattatgagaactgcttcaaatattttctttctatattgcagcactaagtattcttaagcactttgaatttctgtacctacaaatgaaacacagtcagctctgaggtgaaacatggcaaatagtatttttagactggaaatgatctaacaatgacatacagaggatatgaaacaatatggtgtgcatcaaagctgagttctcattaagtatattactatttgtaaatgtaaccacagaagtgaaaataattaaattatgacaatatcagttaaactttttacatatgtcatcactaaatacaaacaaatctaacttatctaatatattatctttcccattaactgtaagctagatagaaattgtatcattgtatgctcagtgactagtatgtcacctgacataaataaggctcagtaaataattggtgaataaagatttaaaaatgtactttgctatcaattgtctcagacactggggtgggggtgggggaagaacaagacatacttaaccttgactttgatgcttctgaagaactttatagaacttatatttaacttgctacagatagcaagttgacttttcaagacatcataaaagacttgacttttcaagacatcataaaagactgtcataacagtctatataacaacatattttaggaagaaatggagataaaacctatttataatgctttgacaacactttgtgctttatgtttcatctttcttgagaagaagcaaactctcctaagtagttttcatttttaaacttcatgtttaaaaatttaaaaaagattttaaatctaaaattactatgctgcaaattctctaggattacaatgatctcagggagggaaacttagtactaaatatgattttaaatttatgtgtttgaccaagcaacacataaaatgtaacatatttatttatattaagcactatattcataatggaagctcaaatctaagacttgatataagacaatacccagcatttaaataaaacagtaattcagaatttagatatttataaaattttatagagtcatatagccaggtttttaatggaattattgatcatcagtatcacttttaatataattcatgatgtaaaatgagaaatattaagtacattttataactctgactatatatgcaaaataaagtgaataaatgaatcacttatctatagcaagttaaatatagttacagagttctggatacatatgaggtacataatatacaccatttgttgaaacagtgaatatatatataccctttcttatccaaataaagtccttaattctcttttctattttatatagacacaaacacacaacacacagtgttaatcatttattgacttgtttgtccctgcaaacaaaggaagtggtggtaatagtcccttaccataatcctacaacttatgtccatttaaatcacttccaaactttttttttttttttagatggggtctcactctgtcgcccaggctggagtgcaatggcatgatctcggctcactgcaacctccacccccgggttcaagtgattctcctgcctcagccccctgagtagctaggattacaggcgtgagccaccacacctggctaatttttgtatttttagtagagacagggtttcactatgttggccaggctgatctcaaactcctggcctcaagtgacccacccacctcagcctcccaaagtgctgggattacaggcgcgagccaccgcacccggccacaaactttgttttgcataagacattatttccaaacctattctgggtaacagttcaaataactcaaaaccaaaacagatttctaaggcaacaaaggtaacctcagacacattttctgtgtcactcatcaactcaaatctgtcatttgttccacttccagtgatttgccagttccctcaaaggcagatgtttcctgtcatctctcatgtctgtgtacctgtttatacccaactctgtgacactaccaatgtcctgcgtatgagaataactgtgttactttcacagccaacttgctgaattcctcttttaacaaataagaaatttcacttaatatttactcaggaaattacattttcttataacatttttgtgtgattcaaagtaggctctgtaactcactgtatctgtgtgtccgtaaaaatataaccacataactaatactttgttcattttatgtatataatccagttaaaatcccattcaaagctacaagacattatacctatttctgttctaccccaattatattagatttcttaagagctactatttcgaattttgttctagagcttactcccaaattttttcctatcagagcaatatagactgctatgggatatactaagctgggcaacattaggcacttaaatatcacaatttaaatgtcaaacacaattagaatgatctaggacaaaacattgatttctttgcagggtttgtgtttctccacattattttctcatgcctacaccgtgactagccctaggtggcccctcaaccaataataaacaaagatggaagtgatgctagccttcatgaagtcattggctagatagtaacttttgaagagtcgtaggtggttagggagacttaagctacactgcagcataaatgatcttataggtgcactaattctgtctttgcctccacttcctcctggcttcaacattgataatttactagtcatcattacttgtgttgtcaataaggaactaaaatggatttggcactgtgctactccaatcagtattatactttaccttggataaatttaaattttatatgtatgaagctaaagctgaatcaaattgtgatgatttggtcactaagattcagtaactgactactaaggtaatagaactgatgttttttagccgaactcatcataatcataggttctttaaacttctactagaaatagtgcatgtttatttctaagttcaaaaagactatactctttttaattgatgtcatattttaccctaaaatggctgcattttagaataaactcattatctgtgtaggattttcaagcacattaatttgtcttcttttattaatgataatagaaaaacccagtgctttaaaatacaatcatatgcaatttatgaacattatttcctataacacttatttaaaaccttgaaataaagtatttgccctgtaaatagctatttttattttattatattataaatgtgttttatttatggtgacatagaataaaactcttaaataattcaaatgtagaactttctggcaagcatgaggtatattttaaaatattttcaagtttcaccaaatttgtttatagttttctgtataaaatcaataaggtatatccacattactactctgtatccaatggtaagatgaaagtggtcaagctagcatgttcagcaaaagctatgcctagagttgagctcaataattcactaaacaactcattgctgtatctccattaccccaaaaaatgccttgagaaaggacctaatgctatttgttgtgcatcttcaaagtaactcagaattgttaaaaacttaaattttgaatataccaatttctgttatatactgtcatccaaagaatacttcaaatatgtggagccattcatctgaaaaaaatcctcaaaaaagtcagtgcataaaaatattatttggatttctgcttctggccaaaattgagtaacagagaccagatttgctctctcctgaagcaaccaataaaacagacaaaatacatgttacagtagttttgaaggcactggacatcagatcaggatgttttaagatattggaaaatggaaaagcaagcaagtcagttctatgcttttgttagcttatggcctggaacttccagactatgatgtagagaggaagtatctctgcagactccatgaatggatgagatgaagctgagtttaggaaaatcagggtggttgtatagttcacaaaacagggtaccacagaggagagagttgagcagagagagaactctagggatcttctcaggttccccttgagtactgagaagattattcaccaagcatgtgtcttagaaaactacctgaggctgaggataagagagaacaccacctggcaatcacactgtgccagaacagtgtctgttcctacactggcaaacacttcgttttaaatgcgcattgggtagagtattaggaagggtcttgattcactagtgaggaataattagtcttagaataaatggtatgctggtcctgcctaacaaattttcaaatgtttaaagatacaaaaggatcaaactgtgtaaacaacataactatgtcccgaaacaaagatcaataatttttatgggaatacaaaaatatccagcacttaaaaaaagtaaaattgacagtgtctggaattcaattgaagattgtcaggaacataaaactgcaggaatatgtgacccataataaagagaaaaattaatcaaaactgaccaagaagtgacacagatgttcaaattagcagacagggaacttaaacagttgttataactgtattgtgtatgttcacaatgttaattagagatatatgaaatataaaaaagacacagatcaagcttgcagaggtgaaaactacaatgtttcagatgacaaatacactggatgaaattagcggcagattaaatgttcaaaagaaaagataattgacttgaagacagcaatagaaatcattcaaattgcaattgacaaatggagtttaaaactcatatagagtctttgctattgtgaatagtgccgcaataaacatacgtgtgcatgtgtctttatagcagcatgatttatattcctttgagtatatatccagtaatgggatggctgggttaaatggtatttctagttctagatacctgaggaatcgccacactgtcttccacaatggttgaactagtttacagtcccaccaacagtgtaaaagtgttcttatttctccacatcctctccagcacctgttgtttcctgactttttaatgatcgccattctaactggtgggtgggggaggtgggaaggatagcattaggaggtatacctaatgtaaatgacgagttaatgggtgcagcacaccaacatggcacatgtatgcatatgtaacaaacctgcatgttgtgcacatgtaccctagaacttaaagtataataaaaaatatatatatatgaaacttagaaaaaaaactcatacagaactacaagggacataaaacagccaaaacagctttgaaaaaaaaaaaaaagaacaaaaagttggggacctgcactatgttatttcaagactcgtcataaaaggtacaggaattaagagagtgtggtactggtataaaaatgacaaataaactaaacagaatagagttcagaaatagacaaactcacctatggacaactaataattgacaaaagttcaaaggcaatgcagtggagaatgaatattttgtttttatgcagttggaataattagatactcattttaaaaaagtgaatctcaacatatcactcatatgttatacaaaaattaactaaaatgggttatttacctaaatataaagcataaaactaacttaagaagaaaatttagaagaaaatactcatggtgttgacttggcaaaaatttcttagatccaaaagaatgatttaaacaaaaaagctaagaaataatactctggataaactgcactttaaatatttgagaagcatatatgtgatataggacttacatcccaaagagtcaaagaatgctcaaaactcaataatatgaaatcaaacaacctaactaaaaaaaaaggaggggattaaagatttgaacaaccacttcataaaagaagctatacagatggaaaataagaacagaaaaaggtgctcaacattagtaattaggaaaatgaaaatcaaaacagaaatgagatacttattagaatggctaaaattaaaaagactggccataccaaatgttggcaaaaatattgagaaattataattctaactggcatgaatcagatttttactatatattcgtgagtcttcacctttcagtatatggatttttttttctgtcttttaatacagtactgtgtttccgtctcttctcctcaacacattaatgtcccattcactatacccctagctcacacagcctttaatattaataaaaatctcaagttggcatctaactttagataccttcctgatatcctgatgtgggctatagcaaacatttttttttttttttttttttgctacagaaatcccaaagaattgttaagcataggtatatataaagtctcttgtcaacctttaaaatcaattaacttcagaacaaaatctcaaatgtcataattaggggtgaatcactaataattggaaatatcagttttatttcttttctaaattaaataaaaaattaaattcctctcttaggttctatgagacaattccatagtaaaccttatctctaatgtattttctatccgctagctgatttctttatatacagtggctaaaaagtttgttagactctaacttcagaaacaagttcatatttgaatacttttttttctaatgacaatgcctatatataaaatgaaaaagcagttccaatctaccctctatagtataacactatttctacttagaaactaataataataatgataaatctttgcttttatcactcagtacctgagcttcttcctgttgctttttaagctgttcgagcatctgctgaaattcggcctctttttgttctgcttcttccaaggtggcctgattctgctcctcataggccatggccaccacagccaggatcaaattcaccaaataaaatgagcccaagaaaatgaccaggacaaaaaatatcatgtatgttttcccagcagcacgtaatgtctaggggaaatgggggataattccatcagtattttagtataatgtccccaaataaatatctgattaccacagttagatagtcagtagactaattagctttttagtatccagtttattttcaccaagaattttttggtattgatatgctcaacacacaaaattaagaatcagattatttgaggcttgactgcagtcttgttggatataaatacattttaaactcatatatagtggatatatatgtgagccccactataatgcttttattagagttcatatgcagagcaataatatgacaagtaagagcactgaacctttaaatcctttcagtgggattccatttgcactgcagctattctccataccatctatagatgacttttgctcccctccccttctagtctcagttaatagctagagctttaatttgaaattttcacatcaacatcactgatttttaaaatcttttaacgtgctgtttatataccaacaactggattatcaacttctaagagtcatgagcatctccaagttcctagcagaatgcctttcacataaaaaagtcctcagtgtacatatataggtactgactcagtgagcaattaggattcctggcacacttgctttatgttggaccttcctctgtgttccagagtgttataagcaggtcctttcccttcccttcccttccattcccttctctccctttccctttccctttccttttcccattccctttccctttccctttctttttttgagatggggtcttactctgtcatctaggctggagtgcaatgatatgatctcgactcactgcaacctccacctcctgggcttgggtgatcctcccacctcagactcccttgtagctagggctacagatgggcgccaccatgctgggctaatttttgtattttggtagagatggggttttgccatgttgttcaggctggtctcgaattcctggactcaagtgatccatccgcctctgcctcccaaagtgctagggttacaggcatgagccaccacacccagcctatgcttttcatttcatacatgtctataaaaataaatgttatgcatgctcatttggaccttaccaactggtaaagattttcccagtagtcttgagtcatgagtcgaaatagagacaggaaagcccagctaaaggtgtcaaagcttgtgtagccatagttggggtttcgaccagccttcacacagatgtatccttctggacactggctataagagagagaaatggaggtagggtcagtttagaaattacagcaatttcaatttatttgacttataggaattttcaaaacccaaagctgctatctgtgacgaattatcttgctttaatttccagaacactttaggtgattgcccaccgtgaaacttatcagatgaaattcactaggaagaccgatagaaaatttagatgcaaataaatatttacggacatttagaatggcagaaagctagcaaccacacacttttggctctgacattaccatatttctactctttacaaagtggcaagaatcaatagttaaaaattaaggccgggtgcagtggctcacgcctgtaatcccagcactttgggaggccaaggcgggcggatcataaggtcaggagatcgagactatcctggctaacacagtgaaactccatctctactaaaaatacaaaaaaaaaacaattagctgggcgttgtggtgggtgcctgtagtcccagctactcgggaggctgaggcaggagaatggcgtgaacccgggaggtggagcttgcagtgagccgatattgtgccattgcactccagcctgggtgacagagcgagactctgactcaaaaaaaaaaaaaaaaaaaaaaaaaaaaaaaaaaaaaaaaattaaaagccagaaaacagacaaagagtggggaatcaaccatttcattagctggttttgtagaattttctaaaataaagttaatatttataacaattttttatttacagaaaaagtatgaggatagtacaggcttcatacattccatgccattttcaatattgatatattgttattatttaattgagatgggatcttgctacgctgcccaggctggtctgaaacttctgggctcaagcaatctcctgccttggcctcctgaagtactgggattacaggtgcgagccaccgcacccagccttaatacattattattacctaaagttactattttattcagatttcatcagtttttttctttttttttgttttgttttgagatgtagtctcgttctgtcgcccaagctggagtgcagtggtgcaatctcggctcaatgcaagctccgcctcccaggttcacaccattctcctgcctcagcctcccaaatagctgggactacaggtgcctgccatcacgcccagctaattttttgtatttttagtggagacagggtttcacagtgttagccaggatggtctccatctcctgacctcgtgatccgcctgcctcggcctcccaaagtgctgggattacaggcgagagccaccgtgcccagccctcagtttttttcttaatgtcctttttttgttccaggatcccattcaggatacacatcgcatttagtagttatgtctttttaggctcctcttggctgtgacaatctctcagacttcccttgtttttgatgaccacaacagttttgaggattactggtcagatattttatagaatacccttcaattgggatttgtctaatgtttgtctcatgattatatttaggttatggatttttgggtggaagactacagaggtaaaatgtcactgtcatcacatcacatcatgccaagggtacatacttatcactcactatgacttatcactcttgatgttaactttcaccacctgtcttgaggtagtgttaggtttctctactgtaaatttactcccttttcttccttttccatatgtgctctttgaaataaagtcactgtgtacagaccacatttaaggaacccgcagttatgatcttaagtatataccaaaattatttgaaattattctgcataggatatttgtccattcatattttatttaattattcaatcatttcaattaacatgtagtcatggatagtcattttatattttggattacaattcaatgatatttcatttatttgttcaaatggcttcatctttggccactggaagctctttcagttgacacctgtaacagtttgacatacaccatggttgtgggttgcatttttttttgttttttaacactttcttagttttaggtactatcagatgctccaggctcatcttttataatttctgccgcagtcctagaatcaaccatttctccaaggagccctagttctttttattggaaaatggtattagaaaccaagatctgagtgctagctgtgtttgttgctactggggtgtcattgcttctaggccctctcagctgacagaacaaggagatacacatgtttgtaattgctcagtactggtacacatgcacagtggtttcaggattgttaatctatactaccattggaaacaactttatcaactagggtattgttccttttacctttagtcttacagactccactcatttccaaagttacttaggttagcatgttattaccccaccacctacagtgaggtcatttcatattttgcaatacatgtatatatttttgtcacatattccatttcttcctgggatccccaaacctcacaaaagattttttttttaatttgtatatattaagattcactctttgtgttataaaggtctatgagttttaacaaatgcattaagtttcatattcaccattatagaatcttagagaacaggttctcaccctaaaaaaaaatcccctttgctttacctagataatccctcaccatgaacccctggaaaacactattttcttatcatctctgttgctttgtgttttctagaatgtcaggtaattgaaatcatacagtatgtagctttttgaggttggctttttctcacttagaaatgtgcatttaatattcattaatatttgtttgtatcttgatagctcatttctttttaatcactcagtaatattttattgtatgggcataacaatttatttatctatacaactattgaaggacatcttggttgcttccagtttggggtcattgaagggcatcttggttacttccagtttggggtcattatgaatacagctgctataaatattcacatgcaggttttttgtgaagacataagttttcaaattgatcagctaaatacctagaagtgcagttgttggatcatatggtaagactatgtttattttgtaagaaaccgccaaactgtcttccaaggcggttgtctagttttgcattcccaccagcaataagcaagagttcctgttattctgcatcctcatcagaagttggtattgccagatacatttttcagtcattctaacagatgtgcagtggtaactccttgttgttttagtttgcaattccctaatgacaaatgatgtcgagtgcctttttacctccttaataaccacctgtactatatactcgtgtggggtatctgttcagatcttttgccagttttaaattgggtcatttgttttcttattgttgagttttaagagttctttagatattctggacactggtttgttgttttgttttgttttgttttcttttgagacagggtataattctgttgcccaggctatagtgcagtggtctgatcatggctcactgcagcctcgacctcccaggctcaagtaatccttctgcctcagcctcctgagtagctggtaccacagatacactacaccatgcctggctaatttttttatttttattttttggagaaacagtgtctccctatgctgcccaggccagtctcaaactcttgggctcaaatgatcttcccctctcagcttcccaaagtgctggattacaggaatgagctacttcacctagccccagtctttttttttttttttttaaatcagatatgtgctttgcaaatattgtcttccagttgctggtttgtcttttcattctcttaagaatatctttgtaaaacagcagcttttaattttaataaaatacaaatcatttatttctttaatggaatgtgccgcttaaatacaaaatggttttgtatttaaaactcatcacctggctcactcctgtaatcccagcactttgggaggccgaggcgggcggatcacgaggtcaggagattgagaccatcctggctaacatggtgaaacctggtatctattaagaatacaaaattagccgggcgtggtggcgggcgcctgtggtcccagctactcgggaggctgaggcaggagaatagcttgaacccgggaggcggaacttgcagtgagccgagatcgcgccactgcactccagcctgggcgacagagctaaactccgtctcaaaaaaaaacaaaaacaaaaacaaaaatctcatcaccaatggccaggcgcagtggctcatgcctgtaatccccagcactttgggaggccgaggcaggtgaatcatttgagatcaggctcccactgattctacattatggtgacttgtacaattatttcattatatattacagtatgctaataatataaataatgtacacaataaatgtaacgtgcttgaatcatcctgaaatcatttcccaccaccacccttggtcggtggaaaaattgtcttccacaaaacagtccctggtgccaaaaaggctggggactgttgatgtaaattacttaagccataataggaattcattaaggggaatagattattatggtttaataggaataaagtatgtattaaccaaatttatccaaaattttatctgggcttttatgttaatcatcatcataataaataatagttaataggcaataaattcttttctttttttagagtgtgctttttttttaaatttattaaaaaaattttttggggggacagggtctcgctttgtcatccaggctggagtgtagtggtgccatctcagctcactgctacctccatctttcacgtttaagagattattgtgcctcagcctccagagtagctgagatacaggtgtgccccaccacacccagctaatttttgcattttgtagtagagacaaggtttcgccatgttggccaggctggtctagatctcctggcctcaagtaatccccctgcctctgcttcccaaagtgctgggattacaggtgtgagccaccgctcccagccatttttcagagacagcctatcacctaggctggagtgcagtggcatgatcatagctcactgtggcttggaattcctaggctcaagcaatccttctactttagccccctgagaagttaggactacacgtgtgcatcacaatgcctggctaagtttttctttttttcttcttcttttcttttcttttttttctttctttctttttttttttttttttttttttttgttttgtagaggtagggtcttgctttgttgcttaggctggtacatacaataaattcttacaattgtccatgtgatagttcaaatactttatgtgtattaaatcattaatcatcacaactattaaagggacagaatattactctctccattttaacgatgagtaaactaaggtacagagagcttaggacacttttccaaggtcaaagagcttacaagtggtgaagttaggatttgaaccctggtatacatgaactctacaacctatgctcttaccccctacattcataataaggctaggaaaactgtcaggaaaaaaaattccccaaagctctatttatatgactagtatacattttcattaactctcttagatttttatggctacaccagtttccctttttaaaggatgactcaaaatatatttaagttccgtgtcctaagagtcacttactattgctatctctcagctcttaaaaaattccctattttgatttaaaattaacttacaatactcttaggaaaatatttaagggttagagaataacagcatgactaagatattatggcatcttttgttgtaacgtgtggcctaaaagaactattcattatgtcaactgatacttggacatgcctttggcacacatgcacccttggcttctttgtatacagaaatacatgctccttcaagaatacttaaatatttcaaagcacttccccaagtgtgcttccaccctgaagaccacattatagtctgggtcttttccagtcacgacaaaactcatcccattgatacatatgtacaccaataaaaaggggcccatcttgtgttcctgtgtttcctaacagctctgatagatgtacagcaaagacagactctgggccttcacacattggagcatcaatttaagaacatcttaactgtgaaattttggggaaaggtcttcacaaagggataatttttaattgctgtgagtttaggaccagtggcaatccggtaaggcagacaaggcacttcctaggggagcagcactgctctttattgcatacatggtaaggctacacatataaccatgtagttgttttcctacctacttttttttttcgcaaagagttctatatcttaaaaaatatattatgtttcttaccctgcatctgagccatttccacagagtaaagggtctttttgcccatccaaaacataaaagtgacctgttaatacaaaaaaaaacccattttatttcatattaatcctattcacattagtattagtaataaatcagagttggactatttcagttatttacaaaggtggctgtacacccacagtctcaactatttatagttgaaaattcattcagcaacactaaggttaacataatgtaatacttcttactgtcatctccaatgtaatccttccagttaaatgtgctcattgttacattaacaaatgtcccatttgaatccattgtgccattaaagtaggaagtggtgttggtttcaaaagcagaatcgcttgggggccactgcaaacatttattcctcagattgcccatgaacagctgcagcccaatgagagcaaacacgctcagacagaacacagtcaggatcatcacatcagaaagcttctttaccgactggatcagggcccccacaatggtctttaaacctgcagagagagaactataggttacctgaggaagagtgccagaaatcatgatttcttaatagtcacacacattctctttcccccataaatgattgcttgactatttagacaccaaagctgtatggatagcctgatgatttgggaaatagatgaacagcagattttacacatgaacagtgtgcttgctggaaattcaatatgaaggtttaaatatctagtcaggaagttttgtttgtgtggttttacatgctgaacaaacatacaaaaaaacttggagttatttttcatttcagattttgtgtttcatgcagccattttccaccaatccattctttactctagcctccaagcagtagtaatttagaggagaaaagaattttaaaataatgtaagtctcatgtctcatgccatatacatgctcttcaacaataatattttctcctttgtaaaaggagatccatttttgaactacatctgcagcgaatggtaaaatccaaatctgttagtaaagccatggtagtgaaaaagagagagagagagcgcaagaggcccaaattagcattttttgaagatgtctcatgcaattttctgttaaactcaaaggctgactttataaatacgcatgtaatttataaaattttaggagctaaactgacattgaaacatcatttggcattatttaacgggatgaactgtaataataagcaacaaggctaatgctgtaagtcatataaattgatttcaaactcaataattaaagtcaacctcggtgtttaacctagctctcacctggaatgactgaaattgttttcagtgctcggagaactctgaatgttctcaacgctgagacattgcccaggtccacaaactctgtcacatatctgtaataggggagttcacacacaaacacaataacacacaagaaaagttggagatataaggggcctactaccttacaccagtttcttcttacctggaattacagaaatagttttcagagctctcaagactctgaaagttcgaagggctgaaacattgcctaggcttacaaattctgttacatacctgcagaattaaatcagagttactgatagttttggcaaagtttatactaaataaggacttaatgctgggtttggcacatagagccctgtgagtttaacaaatggaattgctatagacctcaggctgccatatgctaaaggttgcttttaagaaacaaaatcgtgttgctttatttcaatgtaattttcactaatatgaacacatttgtttgcaaaactaatccaagtacagttgttgctattaaattgtagtcaatgaataacttcattttgatgaagagaaagactatatgacatgaagataaccccatttattgatcatttactggatatatttacattgcaatatgtattctaatatgtattcttaaaatacacaatccatatagttaacttgactaacaagaaagctctacatttaatatatgacacaaagaccttgtttgtactatgactattttcactcctttgcgcttatcaaattttcaaagttactcacgccatcacaatgacactgaaatccagccagttccatggatcacgaagaaacgtaaaatcttctaagcaaaaccctcttgccaagatttttataagtgactcaaaggtatagattccagtgaatgtgtacctaggaaaaacatccaagccaaaattaacaattttgcttgaactgttaaaataaatgttttcaatcatagcaaatcctatccttttgtggtttttcaattcattaaaatattgttccttacttctatttaccaaataatctattcatttagttagttttacaagcctgagcaaagagttaaactgaagagaaaatatattctttaggaatctaatctggtttaactatttgggttttagaataatagtttcatgtatcttatggataatcatctagtttgaccttctagcgcagcagtatctgatagaaatgtaatggaagccacaaatgtaattttcaaattcctagtagcaagcgaaaagatgagaagaagcaagtcaacgtattttttatactgcattttatttaaaccaatatatctaaaatatgaaaattcaacaattaatattagccaaatttcaaggttccatagccatatgtggctagtggcttcctcatcagacagtacaactttagaggcaacatagcttggattcatttacttaaataataaaatattttattcaaatttcttaatgagtagactcattttgagggctagtttacatgtctaccatagaaaatatatttcttttttttcaatttttaagaatcatgtagagttataaactgccccccatttctctctcttctttctctctctctgccctccttcctcccaccctgagtgtatgtgtgtgtgtgtgcatgagcatgctgtatagagatgtgtgtgtgtgtgtatatattatcatatgaaatgttccagttcactagtattttatatgtgtgtgtttgcacaaatgccaaaatacacggatacacaaaattcttaaatcaaaccttgcatcagctactatctctatgcaattgggctaaatcattcaaaccttgcttaaattcaattttcagcctccaaaatggtggaagacttttctatttgctgtttaatgtcatgtatttctagccctcttttctttgagtcttgacaatataagttattgctgttttcgtttttttcccaaaatagttgagagtacttgtttttcaacattgtgttctttaaaagcacaatttaagtttatttttcagagcacttcacccaagccacaaagatttgatttactcatattaaattcattagatttttcagcctaccagtatttatatatgaaaaatgttaggatcattattttgaaatgttttctgcacaagtttctttcatatgagggaacaaagacatcaagaataatttatgcagatatcttcaagtctagttttcttaagtacattcccttccttttctcccccaacaattcaattgatctcataagcaataattcctatctttacttatatgaagtagctgactattaggaagagatatattttacaatatcaagcttatgtttactgtgttgtagtttggatcgttgtgtgacacacataatcaccgtgatatttctgtctgtagagatagctggccttttaaaggcctagaaacaccacacatgtatgttctgtttcttgtattagtggttagtgccctgtggtttatgctgaggcaattctgtcttatctatagagttactaggaataagattaaagtccctacttttgttcttgatttggggaaattaagaaaatatgtgggtacatttggaatgtatgggaagtgttcatggctgtccatggaagagagtcctagagcctactgagacagagctctacacagaataggtattcaaaaatggttattagtaactaggttgggcaggtaagtgggagtcaggggacctttatctttcgtggctatgcccacactaggctcataatcaagctgtcaattctgtctatgttcctgtaactttggtgttttaaaaccttgaaatttggctaatcttaattgttgaattttcatattttaagtatattggtttaaataaaatgcagtataaataaaattaaataaaatgcagtctatagaagtgccttgccttatataacattcccctcactatataatatggttttgtgtatgccacatctttgtacagggactgtttcaaatatcggtgacttcagttttcatctcagaactaagttatcttctgtttcactgaacccagggtttggggtagtaaccactactactttgcttttttcctgctggcttgctttcaagctactgaagacaattgatcagaaaacctgagcaaagttttccagctgaatgatgcaaatcaaaaactttctaaagctaagactgccttcataatgggtataatcatctctaagttattactgcttctttagaaagagaggcctgtatattcaattgccttttttttttgttttgaaacagagtttcgctctgtcgtccaggctggaatgcagtggtgggatcttggctcactgcaacctccacctcctgggttcaagcgattctcttgtctcagcctcctgagtagctgggactacaggtgcttgccaccacacctggctcattgtttgtatttttagtagagatggggtttcaccatgttggccaggctggtcacaaactccaactgccttctttaactatcttttaataatgttatacaaataaatgaaattgatacatgtttaaatgcttcaataatttctgctgggaaaaccacatacagagtagtcttacataggagtgtccattttgttaactccaaaatcatgtatagtaaatgagagttctaagttaaatggctttcctcaatatgggagctggattctctattacaataatttctaggcagcattttgaagctactatttttttttaaataacatgttaaataactgacaaataatggcttagagtggtacagtaaatcctcagagaaatctataaaaacggaactaaaagtggcagtatttgttacactatcttatctccgattgtacattacttttaaaaatataacaataaaaagagcatactgcattttttacttaaccagtcagatacctgattattgtattatactatatataagtagcctatataaccactttgtttcactaaaaactatgtttctttgatgcaaataacctcatatgtgactgaaaaataagaaaatgaaataatgttgttacgataaagaaattgtgttagttcatgcacagttttccccagcatattgatgttttgaaacaatgaagggcattttttctcacacttaaatggaaaatcccttgcaataataataataaaaagatcttgatactaatacacaacatccacagaagtgttttcttttagtgcaacaagggcctggtttaacatcctgaacaactaatgtgcttagacctttctccatgtattaaaatacagaatgaagcattttattttgatgaaatcatacaaatatccccagtctccatttcaagggagaaaggatgaggcctgggatatgaggccacaaagaactcaatggctatgattttaggtacaaacattagaaaagaatcaactgggcaagtgtctttttattgggattattatggtttctatgttgtattctagttacaaagttccataagttttactctcccccaatcatgtttcccctaagccctgttattttatgaacacagttttatggaagacattttatttcttttacagaacacatctaagctgtttcagcacacatgttgtgtataaaaaacttacatatctaaatataacttagacatcttagatgtataattaagacatctacttaatttagagctaaaaacctaaatctattctattcgaatataacaataatcactatatactggaataaaataatttaattcatcaaattactgaactgatcactgtattttatctcaaatgattaaccatgattgagacctaattttaggctaggtgattggtttagtttaccagaaagcatatactaatttataacctctctttaacagatattgtcatgattttgttggaaaaatacttacctcgtagaagaatagctcccccaaagaacttcccttatcttctttcatattatgaaaataactttttcttagacagaagtggaaaccataagtcaggctatacccacaaggagattgctagagaatttgagtgtgcatttctcattcccagaagttattcctacttactctacattctttgtccagtcaggagggttgctcaaggtcataaatacacagttggtcaaaatagtgcacatgataagcatgctgaataaagtagattatagttaaggaataaatgttagtaggttaccatggccatttataaatgatccaaaacacacaaaaaagtttatcgatgcaaaaactgtgaaattaatattatttgttttaaaacaatgataaaataataagaaacttcaaagcctgtgatatggccttggtgcactctttttttcattttataccatatactcacaaaactgaatttaatagaaaaatgatctctatttgaagcaaatgcaagttattttgcattccaatcagttgtgtgaaattgccctggaagaagaactcaccatcccttagtaagtttatagtatgccataagtgtaaaaggtgtacttcagatatattcatcctcactgacttataaaacgttaacgaatgcaatgtaaaaagtgtgagaatgtagagcccagtataatcaaagattacattgattgagaattctatcataaatagtaggtttgtagtgatgtaacttactcctttacaatttatagcataaaagagggagatccaattttgtgctgataaggtaatgttattttctagctttttatttacactttagattgtggcggaccccaacagaccatctggctatactcttgtatctagtccacattatacctaaacagatagcactattctgatgctcttctgagaattagctgtagtacgttctcttgggatctaagtccagacttccctgtaagaaagtttatgattattttgagataaatattttattttagaatataaatactttccaacatcaagccatagaatgagagaattcatacctcaaaaggttatcagtttcatttgccctgtctctaggtgaatcagtctttaagtttatatagaagcctgctttgaatctgcctatacaaaattcccattcttgaacactgcctcagaaaatcaccattctattttatcattacttgagaattttcttgaaatccaaacatattctgcccttttacatatgctttttgactaacattgtaaataatttcaagttcgactttttgatacatattctcaaccaggtacatgagcacatagtaagagtctgataaatatttactgaaaaaaagaagggaataagacggaagggaaaagggaagaatgaaggaagggaagaagacaggaaggacagaaagaaggaaggaaggacacaaagaaggataaatccaggcgctgaagatacagaaacaattaaggcgtaaaagtttgaacacttggtaatgcaaatagtaatactaaccataatctaatctttagcaatggttatatatgacctcattgaagctgataaaggtgtaaggagatttgctctttttcctactatgtgtttcctttgcacattcactatcatggttggttacatattttcttctataatgctgtagtcattctataatactctcattataaatagtggaaaaccaatgcaaataaatgaattaaactaacaatgttaccattaatctcaaagaaattttacagatacttgaaaatataactgacattttctttaaaatcaactgttagaaatagcatttaggcaattcacattaaaaggatatgaatgtaccaaaatcttgatagcaattttcctaacagggtttagtggagttaaaatatacaaggcagaggtggcactgaatcggaaaattgcctttcctttattcattactataaaagtctgaaaaagaaaatacgagtaaaattactaaattagacatgggcttatttaaagagcttacatacatgaagaactttttaaaaaatagaatttgtttgttcaaacttgtttgtttaaaccagttgatatatcataactacaaacatgttagatgtttaaattaacatatagttacaaataaagatactatttactattacctaatctatctctaaggttccaacttgctgatacaaactgacaattctacacaaatttttttatggaattgtagcgttacagattccaactctatttaaagcttaccatctgtcttcagctttggtaacttttcagattactctgtacacattgctactaaaataatcttttataagtattttaatcatatcaaatcttttaacttcaaagtttatgatggcttctgattgtccctgtagatttaaaatgttcaaacaggttgaataatctggttttgctgactatagatgtgtgagaagggttcaacttctcagctaatactgaagagagatggatgcattgaaatttagtctgaaatacaccatttcaaagggcattccacaattaaaattctatgatctgaaagctacaatcatccacttagtattagctatgctacaagtgattatttaccatgttcctatagaatcatcttttatttttttcccactagagtcatctttgctatggccaattcaattcagttgtcagcctcactcacaactagtcaaactctccctatgactctgaatatgaatgtcatagactcctagaagaaggtggcaactgaaaggtcatttaatgcaaactcccttcatttcagggaaatgtctccacaacacattacaatatagttccacctacagtgaccccctattatgtcagaggttcttaatcctgcctgaatattaggctgtcctgtaaagattttaaaattgccaattgaatgaaaatttctgagggatgggcacagatttgggtatttttaaaactttcctgaggcgattccaatatgcaatcaaggttgaaaatcccttctgtctgggaattttctcagctatcaatcacctccttaatcatgtccttccaaataaatcacactacctcctgatcactccctaattgtttctctttaccagtaagtcttagtacattaatttgtactcttttacatgttgcttgtaattatagtcaagtagcaatttacacagtgttcattccagtgagaactaggttagaaattgttataaaccattgagcataaagagcttgtaatctactttttctccatttttttcacagtatttaatagcctggtttgcaaataaacacttgttttaattaaatgtgggtttaccttaaagatactatttacctttaagacatctattctgtatatccatttacttaataatatgtttctctcctttaactaggggctatatgccatacatttatgttcactgacaacatgctttgtttgagtaggtagacaaaaatatttatattgtcagtataaagggcagggaaaacaatttctctgttcattgctcttttgtatttaagaccatggtgccaaccattacttaactaaccattttccagaaactgaacagtttttctgttcattatccctttgtgtttaagaccatcaagccaaccattatttaactaaccatttttcagaaactacacagtttctctgttcattgcctttttgtatttaagaccatagagccaaccattacttaactaacaattttccagaaactgcacactataaatgtgaatagttttatgaagcgggcaagcaaactattctttactagcagtaggactgtgtgtaaaggacagtggaccttggaaagactgttgttgaagcacagaagcattaaagggttgccttggagtcacaagattagattatttaatattacagttgaacagtcaagatccatttaggcattcagcaacacaacagcataacttgaaaaagctcaccgtaatttaaggttcaggtactcatgctcttttgaaacaatttccctgaaaaagcaatttctgcttgcgacattaggagagagtttcattttgaactatctccaaaaacagagtgaacagcaataaattatccctttgaacatggaaccctttgctcctcctgcaagattagccttcgtagtactagcggaattacagctgacatgctaagagcatcttcgttgaatttataggttctaaaacagagcaggccttaagactacaatcccagtttatgtctcaggaacaatacctgacacaaagagaaccctgagttctgagaaatgcagaataaaatactaatgtcctgcatagtgaaagaattgcaagaatttgaaattatggggctctgatgatatgaagagatatacctaaactgaaaattacttgagaaaatgacagatgaataggggagagatagatcgtcattgctacagctggaagtatgttaaaccagctattttgtactctggaatgttacttgcttctttttcctagaatgttacttagatgcacatcttataagtttactaaaagttttactactttgtggcaagaataagatttacatctcaatgattaggaccacatcaatcagagaactcaaaaaatgatgcagccaattttttaactttcccttttattttattatgttaataagcatagtatataatttcacttagtggttaagagctctggctctggagtcgaatgcctggactcagatcctggattcaccacttaaggagtgtggccttgagcaagttacctcatctctctgtgcttcacttccccatttacaaactgagctaacaataggacctgcctcactggttcactatatagtaaatgagttcaagaacttagcagtgcttgacacagtatgagcacaagaaaagttagttatgattatcactgtcactattattaatagcaccctggtcaaattaaatgccattaaaatacattgtgatattactcatttaattaaattaggtattcaaaatgtatcactaattaatgatggtaaagagttacataatgctttaaagcttttaaatgccttttacttgtatttttccatataattaacacagcttcataacatatttttaaattctaaaacttcctccattagcttcttactttactccttctatgctaagaatgacaaattacctccttgtttaatagcacctcaatcttcatctttatggatacttcataaacgaaagcagtgcagccaaccgtaaagactatctagatgtctcgcaagatggttcacagccactatgaataaatcagggtctaagtctggctctcagccatggaggcaataaaaattgactttgtcaaaaacagaggacaattctgttttttagtggcagactcacattaaccaaacttgaaaacctgatatataaaataagaatgccaacctatttatttatttagacaggacctcgatctgtcacctaggctagagtgcagtagcacaatcatagctcactgcagcctcaaactcctgacctcaagcaatcctcctgccttggcctctcgaagccctaggattacaggtgtgggccatcatgcctggcctccagtgtatttatttacaatgagatacattactttcatatggactgagataattgtagtgatgtaatgagcacgttcttttcttcaagcaggtttaggggaaaacaaattgtagtcagaaggtttcttgctgaatatggagcccaagccagctgacctaatcaaatgatgtggaatgtttaatagcatgtgttaggtagttcagtgtagcccctccacaatgaggtggcctggccattgggaagaaaatgctttcagtgctactactaaagttctgtctcccttggaaaatagtaacgtttgcctctgttgttctccttttgtttcccccttaatcttaccagcctgattgattctcttttttaaggacacttcctttctttaagtagcaaatacttttaatcatagcagagtaaatcatgcctcctttcttaaagatatctttacaaacatatccttcagagcaacagttttcaccggataatattttgtccaatttcttggggggtgacttattgtaatcatcataactacatttcaaatttttctttatcaattctatagtaaattgttgaaataagttatttttagaagtacatgaatgtattaaatgtcaccttttatgaaactagctataaaaggggaaaaaggagactctgtttagctatttacatgtagtcttatgactttaaataaaacattaaagcttgctattcaaagtgtggccatgtaacagcctgatgatttgtaagaaattcaggatctctggccctagcccagacctatggattcagaatctgcaaattaaaacatcttgaggtaatctgtatggatgtgcaagtttgagaagtacttatttagaacaacaggactaattccatgtctgaatggttgtcattgcctttagccatcattgattttataaattataaacttttaaaaggatgtgctttgactaataaaggtgtttaatatcttccataatctataagtcatcaactcatggtgatactaatttatctttagtttctacattgagctatttttacgcttttaagtgtaaaatataaatcgtggttataaaatgccttaatatccaattagaaagcatagagtataccacctattttttatattttctataaaatatcacccagctagtttaactatagaaaccagctaattctgatacaattatgcatttattttagcattaggaaagcacagtcataaccactaatttaaatcatgtatggatggagaaaatttgaaagttctttctttttggtcttccaattttgacatttttttcttcagggtgatgttttaatggtatacatgatccagaatctagcatgccacaaactatataaggttaagagaagatcctaatcatattctaggttactgggggggaaaggaatctctgggtgaacatgcttcttcatgtattgtctggctacaggaggatgctaagcagattatttattgtcctctagaatataaattattgtgaaggtacatcaagtcagtcatgaactaaaaaataattaagtacaacatatgctcataattcttttctgcacaggtattattctgtagtctaccctgtgcttgaatatgtcagccataacagatatttcactttgttgatattaattttaattttgactatctgaattatcactgtgaaattgtagaaatcattcatctattcatatttcctgactgtaaccaactcttgacttcatttcttataaatgtttgttttattatattgtgataaaaataagtgacagtgaagagtattataaaatctttaacagaagaaaaattgtgatgctgtatataaggcccagaaaagtatattacagttaagagtttcattaaagatttattagaagtctaaaatcaatactcactttcttattgatatagtagggatccaggtcctccaggggctctgacaccatctctggaggaatgtctccataaataaatggaaggttctttccagcttccaagtcactatttggctttggtttgttctcatcatcattatcttgttcctttttgggcttcttggctttctcttctgcagcacgtttttcgatagcagcaagagattctctagtaaaaaggcggaagctttcaggtcctgggggtaccaacagtgcctgtgccatcttttcatcctgcacatttaattacgtgtagcttcttgcatacgaattacctgcaataaaagaaaaattgcacaagagttaggaaagcaagcgattgggcataagaacatcaaccaaaatgtcattttttagtaactttttaaagctataaacattaagtcatgcttttagtactgcactacacatttgaacttgaaaacatcatcattcaacctcattctcaataagccaagttcataaatctatcaaaagtctgtacttaatgttaattagtttgatttagccattctacaatgtacagtatatataatatataaacattatattttacctcaagaatatctataattttgtccattttttaaaagcatttaaaaaaattctgttttgacactgtaagccaaaatatagtatactttcagcaaaagagtaatgccagtaaagtttgtcaataaaattgcttaattattctgcatcctgccccccgccttttcataactttgtaactccccaaataaagaagtactggtaacaatacaattattgtaatacatccctgactcatacaaatgaggatagaaaatgaagttttgccaaacccaaccactgttctcgttgtcagcatcattgacaactgctgctccagaaaagatctgatcaccaaccagcagggacatggaagccactgctgccagaaaaatctacctatctttagtatttcctttcttttattaagccagactctgtttccttctgaggcaatggagaatatgtttactccttatttcaatatgctgcccttcaattattccaagacaattttgacgttttccctgaatcttccatttagtatagtggttaaaagcatggactcaggtgctacactgtccgttctaaaccctggctctgccgcttatgagctgtgtgattgtggacaaagtaaccccttattgcttgaattcatctataaaatgagggtagtagggaaactgacttcctgggttatcataaggaataaatgagttgattatgttgtgtctagaatagtttttggaattagtaagcactatattattgtaaactatcataattaaattatcttcagtttccctagctattttccctcttttatggtttcaggcatcctcacaattcaaaccactctttaaacaagttcttgctgtgctggaaccacaattgtgactacggaattaaaggaatgaactgagaagtgcagtgtatagaggcacactcacacatgccgatcacctggatgagaaagaagggaaggcagactgatggagtggccctgctcaatttctcagggaagggaacaaaaatctatttgcaggatacataatagaactatcaaaaccacaagaaatcagctatgtaaatttcgaaatatgagttattttattttgcattatgccaaaaatcacatttaaaaaatcaagatagatagaatatagaaacaatgacaaaccaaataaaataagtacatcagtcatcttataaaagaacctaccactatggcaccaaagtcatgatcaaagtcatctgtgacattttccccattcctaccattcccctttgcccccacatgtattctaggctccgtagtagtttatccctaaatggccctctgattcttggcagctcactacatatatttgagcttacaatcagaagctctggatctattaataacataagaaaaacaatatagcttattatgtgtgtgggggtttttttgcttatatcttaattactagttccctgatctattccaatgacaaaaaagtgtttctataacattacttattatattataatattattatttttttgagacagggtctcactctgtcacccaggctggagtgcagtggcacaatcacggctcactgcagccttgaactcccgggctcaggtgatcctcccacctcagcctcctgagtagctaggactacaagtgccctccaccatgcctggatcatttttgtatctttgtagagatgaggtttcagtcgccattttgcccaggctgatctcaaacgcttgggctcaagccatccgcccgcctcggcctcccaaagtgctaggattacagcggtgagccaatgcactgggcccaaaaagtgtttttaacgtaagatatgtcttttctttgagttagtatttatgatataacattgtcattatatcttttgctactatgaaggtatattctatataagttataaaataaaatagcaggcctgtcaaaagttatgttatttttctctactaattttgaacagaacttctgttttatagttgggcagcacatttttttttctccatcaggttgtatagtacatgaatcgaaaggtttgagaaaaattctgttttactcaaaagtcagttgatcatttattttgtgttttctaaatttacgggaggcagcacaaaataaaactaatgtttttagccatcttatgtcaattttgatgatcacatcatcaatccaactctaagctgtgtggaataatcacagcttttatgcttttgagtgcagaaagagtttgagcattttctgcaagccgtaagtacctaaagatgtgtaatccatctgtctgatatcacctgccatgaactgaatggctgtggctcttctccagtgctgaatgaaaatgaaacagcatgacaaacatgtcttaaggaaattctatgaaacacggtcgctttcttagaggagaaaaaaaaaaaggttgatttgaggatttggttttgaactaattttgatgtctttattgacaatgaatgaaaaacacactgcaattgaaatgggagatctgggttagtattgagtctgctgctaatggtatgcctgtatcatctaacctgtctcagcctcagtatcattgaggcacctcaacacagaaatggctgcccttttccagatgggaatagctaatttatattaatacatacattttacttaattggattctcctggttggtttttgttttagaaataacattaggtttaaaatatctaatgtggaaatcaccgagtaattttcacaaattattctgatttatctgagatgatatggaacaaggtgccatcacaaaatcatgaaaaactttttacaaataaatagtcctttaccttcttatttacttatcaagggctggaataaagaatttgaatcttggtcaaaagaaagtaaaaacgtctcgttgaaattcccatactgttgtctctaagtcctcggaagataggctagttccagactctcagcttttcccctgcaagagaattagggtttgcccaatatttgggtaggctgagaaattaaagaatttagtgagtagagtttctcatggcttcaatgacaattttgctgtttggaaatccctcatagttctttcgcagttctcttttacttttttcctctcctttcttgtactctgtgagcttattatacagagttcttacataattttatattcacattagagcagactttaaaaattaattcttactgatgccttggtgtggaatatatatattattaatgattatatgcacattataatttattaaaatacataaaatttaatatatcaaattttctacagagctatctttgcaaggacaaaaaagatacgaaaaaaatcttaggagatgatctctttaactaaaaaccaaaaagcctgtggatgaatgtgtgtaggtaaagcaaatacatccctttgttaactagtaaaaagattatggttcagtatatcatatttgaggaataaactgcggaactccaaatctggaaaggatctcgaaatcatttatttggaaaatatttatggagggattctacgaatcagtactctgtttgaaccaggaacccatggtcacttccctggggctgagagtccaatgtgggaggcagatttttatacagataattataacacagttatatatggctggttattgtctgaagttcataaaaagtatcaatatgttcatcccagggaagggagagagaggtatggtttggagaaatttccctgaggaagtgatattggagtaggttttgtaggctagttggaatttgattggggtcttatggtggtagtggttgggaaaggattggtaggaagacacatcagtagagacacattgagcaggtggggtgggggtggcatgtgtgcagatgcagagacaagaaagagcatggagagtttgggagcaatgacaagcaattcattatgggtggacatcaccctgcacagaagggagtggtatgacataaaactctaaagttaggcaggatagatcactgaggaccctgtactacacacgaaagtatgccatggatggggagtgttatgatcaatgtgaagattggaaagatggtggcagtatgaagcgtggacaggcagggagtgatcctagagcagtagttttcaaatgttgtggtttcaggatttctttacactcttaaaatcattcatgaacccaaagaacttttgtttatcgggggacttagctatccatacttaccatattaaaaattaaaatgaaaaaatgtaaaaattcttattaactaataataagcacttgtatgtttatacagacaatagatggttaatgaaaacaactatattattcaaaacagaaaaaaatttaatgagaagaatttgtttcacatttagtcaaatatcttcatgtcacttaagagaatacatttagattttcataaatgcttctgtgttcagtctgatagaatatgttgttttattgaaatatatgaagagaattttcacgcatatgtgtggctggaaaagggagtgttttaattgcctttccaggtaactgtgaatatccttctttaatatcacgtcaaaactctacaaatggtagtttattaaattagttgtgtgaaatcaaaagccatatcaatgaacttttgatttatcttgtcctttgaataagtcttttacctaatcatgcactaattatttggaaaatggcacattgttatgaaggtcttccagattttgacaaattttattatataatatccaaaaatcgcatttgttactatcaccactaatgtcatgagaaaagttgtaatgtatgggtaggctgtcaagctcccagtggcaggtaaaagttttcccaaattccaatttcctcttgaaagctcgaattttgacattgacaacaaatgctgtcatttgcttttcctgaagtgacaggctttccctttgttcatttttgagaaaatgtgtgccgaatactcaagtctgaataaccatagtttgtcagttattctttcaagtgaaagtggtgttccatgaaaaaaagaaaagtggcttattcagctcctgactcaatcacacaagtgcttttcctagagacaactaggacacttcagtatgcagtcaaatactttatatattcctcctatttcatcatacagaatgttaaaaatatgtatttttaggattgtgcttgaatgaaattaatatgtactgcttcatcagagatgtattatgtgaactggcattatttttcttactgagagtgtgcagcaatgaagaaaatgcagactatgcctatagtttgatgctactgcattgattcctgctaaagcattagcagttttagcacatgcctattgctttatcagtgcaagtgccaaaacagtgaaaaaagacaaatgccattttggtattattacgaaagtagtttagagctcaaagctcccacacaaagggccccaggaatccccagttgtctaaaatctatacttcgagaacaactgccctagaagaaagcagttagtcaccactcaatagtcaaaagaagagaagtaaaagatctgactatagctctggtaccaaaaggcatggacttagaaatcagaactggattcaaggcctgactctgctgtgttcctgaagcttttttattctttataatttccctagctgtacaatggagataatcatagttcatactttatagagctaatataacatttaaatgagaaaatgaacctaaagggtgagcacagtgcctggaatacattaatagtaggccatcaataagttagggagaaaaaccacagccagaggagtgtatttaatgcacaagtataggagagagaaaaacaatgagattttgtgactgacttgataggagatgtgaattaaagttatagggttaagctcacttccaggtttccagcttaagccacaaatcgacattaacccaaatagagaaatggaggtttgtaaggcatgataataaattctgtttggaacctgttcagtttgagatagctgtgagacatctggatatagaattaaaatatggatctgagactcagaaagctgtctgggctcgggtaaagttgattaagaagttactggatgagtctgggcatggtggctcatgactgtaatcccagcactttggaggctgaggcaggaggattgcttgagcccaggaattcaaggctgcagtgagctatgatcttgccagtgcactccagcctaagctgcaaaacaagaccttgtcaaaaaaaaaaaaaaagaaagaaaagagcagtcactggttgaagctgaagctaagaatattcacggcattgcctaaggaaaaaaagtgaaaggatgatgagtgaagaggcaagactcacacaactaccaatttttaagtaaagggaacagagaggatgatagacaatgaagttagaacaattacctagatgccaaagaggaatttccaagatgctaattaaaagacatacttgggatttaacaattagtccaactctctgcctgtaaaataaatgaagaacagtcccaaagaagctaagtaatctcctctaaaatgtctactgacaaacacaaattatgaccttttctattatagttgacatatttgtcttgaatcttactattatatgatttgctctaaaagatctaagctgtatataaataactacagaaaaatagatttgactagttcatttcagttttctagcctcacctatcagtgtactatgatgctaacataatcttatgaaagtacttgaagccagccaatacaatgagtacaagactaatatttgttaatgaatcagaatctagaaaaatagcattgtaaacatttcggatatgctgtggtgttactaaggaaaaatttatttttttccattagtcatggcctatgggttctcatcttgcctaattttccagaacagcagacagtttggcaaattgtattggtccataacctgtagtattagtagtgtctctctttccctctctctgtcgggatttttatgattgtgttgcagtcaaaatcgattagttctggtctgtttccatggaaacaagattgtgtcagaaggctgtaaatttttcatctttatggatggaaagttgctgtatgtcatacaacttgagatagcattaaaaaaatctgtaatgtattttcacgtctttgagaaggagagtgtgagttgctgtgaaggactgggtccattactgaggagggatgctgtattttcttggtatctacaagagactattatagtaagggtataaggtaaaagacaagattgaccgatgaattacctgttgaagaaaattagttatttttcccagtgaaattttcactgagagctaatgattgtctgttatttaccatatattctcaggcagcatatgcacccaacagcataagatttatcaaaggcatacatagagcactcagcacattataatgataaccactggacacattccaaggcagaattaaatactattttctataatttcaatagttttgaaataacaccttaatcagggggctgtgaagattgaataacaataaacaaaattctattgaatgattactatatctcagacactgtcatgccatatatgcatgttagcccatttaatcactgttacaatttcatgaggttgataccattaatatttccactttagagatcaagaagcttaggtgcaggtagactagataagacaaggagccaagatttaaacctgtctagttcagaaaaaaaaaaaaaaaagaaaaaaaaaaaaaagcctaggtacctaagcctgcaaaatgagaagattcttgatgtttgctgaaacaaaattaggataaatcagctaccttactgggaggttactatgtataaagccctgtgtcaggaatgagtggtataatctaagaaatgtagcaatatagtccttatctttatttccacataggagataaagcaaacaggaatgaattaataacaaacataaaacccacgtctactttcacatgtatgctacacactttggagttatttgatgataaattaaaaacctaggctagcttcttctatgtcaggtacctcttgctctcagtgtgttgtcaaggaaatcagcattactattagtctgaataaatctattgagaaagtaactctacagcaagggtatcactacgttatagccaaacacagatgaaatctagaccttctttatgttattgttccgtttctattagctaaacacatgattgccaacatgtgttaactatttaacgagatgatctatttctgtttttctgtacctggactcaatgtcatggctctgaaaaaaaaaaaaagcttagtaaaaaaagcatgggttcatctctatcttcaagataattatcctggaactaattaatttttaggggttgatattttctgtgacttcgtccttatcagatagttattagcttgaagagacaactgctaactagtaacaaaaattaagtcaacattttaaaggacaaatcagcatcactggatttattatctctttaatgtttctattaaatgcttgcaaagtaaactgtgtgtttgaccccccagtattcattccattccagatgtttccttcaagtcccttgcccatctttgtttaaggaccaggtatatgatctaattctggccaaggaaaggtgaggggacttcagtcaagtcattgtctgtaaaaagtttcttcactcccaagaaagagaacaaagagaagatagcattctctgtcctctggacccttttctattgggataaaatacatggcactacttcagccaaattaaaaccagaagagatgccaatctgagagcaaagctgacacactgagggtggcaggtcacagagacatatttacccctgggcctttgatggatcactcagcttgtggacccgaacaacctctgcactgcttgttctgttcaataaacatctttcttgactaagacagttttagacagtttttctagttagttgcagatgatacaccaaataaataaatagtctttggtcagttcaaaaacttgaccaattcagaagccagctcacaaccaagtactcaccacatatttggatgatctttaccccatacagtcaggtgagccatagatccaattaccaaataaatataataaaatttaaagtaaatacagtccagggtaatttactaaatagacaggatgaatgtctctcttaaaagaaaaatatataaaaaactcagttgctgggcgcgatggctcacacctgtaatcccagcactttgggaggccgaggcaggcagatcacgaggtcaggagatcgagaccatcctggctaacacgatgaaacctcgtctctactaaaaatacaaaaaactagccgggcttgtggcaggcgcctgtagtctcagctactcaggaggctgaggcaggagaatggcgtgaacccgggaggcagagcttgcggtgagcgaagttcatgccactgcactccagcctgggcgacagagagagactctgtctcaaaacaacaacaacaacaacaaaaaaacaaaaacaaaaaaaaaactgagttaatctctctttcttacatcaacagcctttggcatcattccattccctaaggtcatctgtgctatgtctccctgtatgtatttgcctctttggaagaacaaactggtagaggaaggaagaaaacacccctctgccttcttttggtcctgatacagctgtccttgttaaatagtagagattttagtttattttccacaaaaacaaacttatacccagaagttgtttagttaccttagatagtcacagcacctttttccggaaaagctccaggtcccttctgtgaattatctcatttattcttacaatatccctagaagagattctttgctcctttcccagtaagccactctactatgaatccttgacaggtatctcatccctgtcaaaccttgatgtggcttggcttcagttttcttgctccataatctctgtagaaagagaatacaggtgaaattagcctataatctttgacatttggcataggggtgctctggctctgtgtctgggttttattccttttttttttatggccctggatcgagaaagcaaggatgatgcaaagcaagatatttgacccttgttaaaagcatgccctagcatgatttaggaagaatgggaccaggaaccactgccaggatcctagggttgggacaggggtgctcccccttctttggcagccttgatccatttagtctttctgaaggcatgctggcactcctgagcatccttctgttcttccctcttgcttccttctctcctcttccctccttacatcttcccctttgcttcttttcctcaaacatctcaaagaccaaagaggtagactagtggaacatgtggttcacattggtgttgtaggtcattttctagttccttctcctctccacctaaaacccagtttgattggcttactaaaataagataaataaacctatttagtagcttctaagagtatattttagacaagctgaggtaaaaagccagcatgctaaatattaacctaaatgtaataaccagatgtcatacttctttccgctataaccaacagcaatggcttagcagagttaaaccaataataataatttcaaagggcaatatatataattctaaaatcagtaactgacatttaaaacaatatttactgcaattcatttatacaatgctcataagccaaactagtcatgaattctcaccagtaaatttcccttggccacttaccataacaaaaaaacattaaatctcacttttacaattttatacatggtctatacaatctttgtctccagatgaactctctcctaagccatcattttttttctctattctgttaccggagtgatggtttcaaaacacatatcctacccttgttcaaatctgtcagtggctctcagtgactcagaatcactttagcaaagtatggaagtgccttcatgatctggcctctgcctctcttctcttacctctcaccaagtggtaacaataacacaaatgctaataataatacccataagacacaccttgcatcagaaattgagcaatagattgttacataaatttataattcttcaataaatgttagttccctctacttcacagccacaagcttaataaatgttatttcccttgtcttcatagtacacaagtaactaagttttaaatactttgaccttcatcctgagctataatgaacatgatgttaagtatgttaaataaccaaaacatcaatttcaatacatttttaaaatatttgagagctttttgctgggatgtggagggggatgaggtagggagggctggatatcgaagagggaataaggaggagcagaagggtggagtttgaaaaagcatcccagaagtttctgatttgcttgagttatctttctttttcctacccttatgaaaattactgatttacagattggagaaaatatattttatggccttggcttcaaagaggagagagtatttttttttctttttcaatcccaagtatccatgggaaatcgcagtgattcctgcatgtggattaatacttcactttaggccacatacttcaaactgcgttatatcttaataagagttgcatctcacttatttttaaaagcagccctggcttacaaattaactattatataaaattggactctaagcaattctgaacttcagtgagcataagaatcacctgtgcagcagggcgcggtggctcacgcctgtaatcccagcactttgggaggccgaggcgggtggatcacaaggtcaggagtttgagaccagcctgaccaacatggtgaaaccccgtctctactaaaaatacaaaaattagccaggtgtggtggtgtgcacctgtaatcccaggtacaggtactcaggaggctgacgcacaggagaagtgcttgaacctgggaggcagaggttgcagtgagctgagatcacgccactgcactccagcctgtgtgacagagcaagattccatccgccccacttcaaaaaaaaaaaaaaaaaaaaaaagaatcacttgtgctacttgtaaaagatgcagatttctggattcaatttctggagattctgcaaaaagtaggtctgaggtagggtcgagggatctgcatttttaatacactccctaagtgagttttgtgcaagcgatgcatggatgactatttgagaaaggatgaactaaaggaatgagtaaagaatatgatctgagtgcttttggcaatcaagcctggaattttggtgtaatgtccccagtaactaagaaaaaattgaagcatattggatcacatgaattttgttctattttaggaaaaaacaaaacaaacacatttcctaaaagttgtatataaaaagcaaacatttttcaagaactaaactgaagggtggaaattttgtttaggatatagtataaaggacatgaggcaacatgctcaacagacatcttaaattttattttgcacagatgtgtacaagatgcaaaaaagtctttctctgatggttgcaattagttaaactaagtgaatttcatttagaaggtgatgagataagatggtccagaaactttgctttcagatgacctaatacatttattggatctggaaatcccccaaaaacggttagcctgcatttgaaattatcttaataaaatttcagcattttagtaatttaggagaaattctttggttttcaaataaggaagtgatagctcaaacagcatgatagaaattttaccctaaaaatgctagatgtcaccagctgaggaatcttagacaaattaaatctttaattttatttttcttaacccgtttctacttagtattgttattaatgcttatgctaatatcaacaattgaatattctagaggcaagtttgtatcctcttttgtggttattttagtttatcctgaaaatttatttatcctttaagatcttctagttcccagtgttgtaaaatttttgtcttactatggctccattattttttagcatcattttgcatctttactctttatctctggccatggaagcaactttattttctttcaattgtttttatcttcctttatactgatgacatgatatacctgcttaagaatttttcaggttacaaccatttgtgtcatgcagtaggctgtaaaattctggctgttcagcattttgcacaagaacagaagcagatccaaaggtggtcatggaggaactcacaagctttgccatgaggaagggacagctttccatctaggtgggaatgtggttggcttttcaacctctgtgatgaggtaacagtctcaattaccatctgctcacttggttttaagacttcaggtccctatgaaagaggtaacttcaaatgggcaaatcaccaagatattagagggtgaaatattgaaatgttgtctaggaagccaagttgtccattcagttcttggggagctccagagattctccttgtagtaacttcgatcaccttcgtggtggttcatgttaacttaaagtctgagacttctttctttctttgtgttgtataaaattcatttcttcctttaaaaatgttgagctcaggaggctgagagctaagtgtacattttgaaagagtaaattcattagcctacagtccttaacagatgtatcacatacattttctccattttccttgtcttgagattcctattgcacaaagatatatatatataactttatatatataactttatatatataacattttatatttatatataactttatatataactatatttatataaaactttatatatatataactttatatatatatatataaaactttgcatcactttttagaagaggtgagatataataaataaaaatcaagttcctctatgtgatttaaaaggctttttcattctttagcatgattctatacttcttataaaccatttttttctcaaggactatacttaattttataataaaaataaatattaagaaatgaacttggaagcactgtatcctaacattgaaaataatctttcaggccatctagtttcatcaacctagtttaaagcagtggttctcaaaatttagcatcagaatcaccgggctaacttcttgttaaaacacagctttctgggctccaccccaggtatatatggctgggacccaattttgcatttctaacaaatgcacagataatgctgatgttgccagcccaggaaccacactttgacaatacctagcttattgaatccaggattttacgttgttttttttttttttttttcaggttagaaacatgtatactgggcatatcactttcatttttgcagtttcttaaagatgacaatcatagcttcttcagtctcatttatttattcatttgatgccctatggtgtaatttcgctgcacctggaaccttgaaatgatttacagcagtattacacagaagcctgcaattgctttcagtctccctgctgccagcctgactcccatatagcctgtttttaccttgcctgtcagagtgatttgagaacatagaaatcgaatcatatcattcctctgctcagaagcccccagaagctacccagaaaaaaagtcccaaatccttacaattgcctaaaaggccttataatctgtctgaccttttggttatctagtggaaggcacctccttctattctcctcctccctcagttctctgtagcctaactggcttccagctgtcctctgtacctggaagcacacacagactattcccttattccagtatgctcttcctctaggaagctgaatgactaactcttctatctccttcaagtcttggccaaaagtttactgtcttaaagagacctgctcacacttctcctctaatctcttaccctgctctgatgtttccaagtcacttatcattttgttaggtactatataacttacttataatgtttattattcattttatgtctctcctggatagcacagaaacagcacaggggctggaatatttgcctttttggggtctattttcaccaatgaatctcatgtgtctagaacagtacacgcacatagtagtgctcaatgaatacttttttaaatcaatgaattatttaattttggtttctgaaaaatttagtagtttttttttttagatttattatttggaaattcagcccaatctttagaaactcacatattctatttatggcaacatattttataatagcaatgtctagaaaaaatgaaaccatccactaatggggtattgattaagtaaatcagataactttgttttgtattatatagatattaagatgataaaacataaaaggactcaatataggaaaaacaaagtcaaactttataggcactatatttattaattatgtagccttatttaatctaaatcaaaatacatacattacaatcctctttacctcaaaaataattatataactatgccaagtttactagactctcttgaaaacaaagcaaattgaagactgaatactttacctaaagtatttcccactctaaaatatcttaggaagtaagaatgataatagagaacagtgattaagcactggtcacttatcagggactgattacttcatttaatattcataacaaatatataagataaacatgattgttgtttgtattttttagataagaaaactataatgttcagaggggacaagtaacttgcccaaggtcacatgactagtaaatgttgccatcaaattctataattttttctttgggaaaattaatcaaagcctctctacaatgatcttaccaatgaacctctccagctacatcttacatactctgtatatttccattccttaaaagacctcagcctctgacatctggcgttcaaaaatgttcacgctgtctgaaatatcattgaccaccaactgtcacctcttaaaatcctactaaaatttttacagtaattatatatattctatcttctgttgttattgaagatgtgccttattgcttctacatttattattaaattccttgaggagaaggatgtattgactacctaccacagtaagcagggcattatcagatatatggtggatacaattgcccaaacaaatcttgcaaacattctgttcaacgtggtagccattgaccacatgaggaactgagcatttgaaatgtgggtaggtgccaaatttcaaaatgatatttaggacatatcatcttaaacaaaatatatcattaataaaatcaatcttattgttttctttttttgatattaaaatctagaacatttaaaattacatacatgggactcattatatttctaacgcactgtgcttttttcagatatacctgttccctactccatcactctcctccaagggatcaaacccatgaacaaatgcatgaattaagcaagaggcgaatcccttattctgagatgttctacatgggggagggggctgtctctggatcaatagtttttctattttatgtagtcaggttgctgcttttgtcttttggtctaagaaacaccatagtgaaaaagctacttggtccataagtgaacattctctctctgtgtgcgcagtggagcagaaaccacatgcaaatcagaatataacagcattacaggaagctttctattttgaagtacattggcaacatgacaaatataaagcaaacagcattctctgaacaatccttgatccctttcctaccccaacatcagccataatagacagccctatgtgttgccctcagccacatgtctcctactgcttgctggtaccatgtgctttaggcagtttctttccaggcaaacacagactgtttactgactgtaatcaaaatgacagcccactgttttcacaactgagtgttgagagtcgattgcctttttcacttggcaacctgaggaaaattgagaactgggaacttgaggtatgacttacattacttcaagatggcggtgttgactactccacatgttatctgcgctcctttgagatttaaaatacagaggattgggatttgtctagctcagagcaaagaggttttaatcactgctttcagatgggtgcaaatggtaaaccacactggagcacaggatgatttaggtgtgtctgaagttcagctgccatttgattcttcatcagcaaaaaaccaatcacataaagaggccttccccactaagcaggccccttagatacacagttataatgttacacagtggatcactccaagatggtgcctcagaagataatgcaatatactagaaaaacatgcccctgccctatctttattagaaatgagatttaatgaaaagataagagagaggaaaacagaaggagagatatacaagttgcaaatcagacaaactattctagacttgtctctgctgtcacttgtgtgatatattgaacacatgagatctcagatgtttcgtgtttatgtgagtaaaaaagaagggagaggtgggaaattgaccagttaatttgtagtacctcttttatcactaaaactctagttcagtgtccagactttgggactctgtactaggcttactccaaatgtttgtcccttctgagagaaacttctgagggtatagcaatacccttataccctgaacgtgggtcactaaaccttatataaatcatgttcctgaggtaggtgtgtcacatctattagaggatatattcatttttagaagactttagtgaactagatgcttgcataggtcctctgcagcccaaatttaaactgtttcttgataaataatataaatgccttgagtagaatataacttgaagatcagttgctgaaaagaactatcccttgaatcctttctaactctagatattcaattttgtacacatgacatcttcatgaaatggtaatataaaaaatctcctactgagaaattccatacaaattgcagttaaaagaaaaacctagaaaactttaagtctggttttcagtgaaatatgaccagtcacttgaccatatctcccccaaaaagggccctttgaaataaagaaaagatgaaaaaaaaaagtctttcctgggatctgaattcaggacccagagaatctgtgtttttccttcaggctccgagcacatcattggcaccaaaatagagcaaaggactgtgtgcactgccctgatttaggtgtggatgatgccacttactgcagcggggaatgcagagtaaaaggtctctgcacataaggacaggaaggggatgctgttcagtgctcaattcttttgaggctaaaggagatccagaattccacaggtaagagaaaattatatatgataaaaaattttgttttaaacagggattgtagcaaccaaactgctgaagactggcgtggtctgtctgtcttcaccctccagaggatagaggacctggtaagtaagaaactgactagtgagggcagtaatcctgaggagaaacctaagaagaggtgagagggaagatgccaggagggagagatgccctgccgtgtgccaggtgtgccacctgagctgtctcctcaaggacatggaggattcaggatcctgataaaattgccagtgcaaaatcagacattgagttcactcattcttcaagtgtcactcctctttaaaaattaacagactttttttagaacaattttaggtttacagaaaagtctatcagaaagtattgagttcccacatcgagtgccctattgaatgtcttaaatatctgtcttcaaaatttttattgagaaataaattgagagaataaatagaaactattttgttaagtgtgaaacttcccaaatttagctttcttgagggatgtgtattgttgggggagaggaggaagtgtattattttctgattaaattgatctcacagtgttttctgaactggcaccactatcatagatttaatgggatatttaaaagaaaggtgtcttacaatgacaggataattttaagatactgagtagaaaaccattccttaccaatgcataagagggagctgactattgttttcatggtgtatcttaactctagtgtttatttgcatgaaaatctgcatcttgatttggatgtgctctgccacttttcatttaccttcatgctttaccctttccttttcaaaatgttcaaagtgcttttgaataaggagttggaatgtttcctcaaaagaatctagctagttcttacaattgtcatcatgttggtagaggggaatggttatatcaaccataaattattccccctctgcactgattgtgttatttaaggtgacccttcttcataaaagcattcctcccctggttcaggctttcattcttatcaatatctcattggtttcttatttatattttcattgcagacttcccaaatcctttgtgtataaaggttggatttttttttttaaataaatgatccaatcccaatgggaggacacaagcactgtgtcacacacattcatcataaccctggcctcgaacatgataactttgcaattccttggccagtatttcttctgtttattacagaagcaccagcgacctgcctcaaaatgtggatattctcatcttgagatttaacagatattttctgttatgataatcattttactgaccacgttggcagaatgttagagatgggtgaagaaagcatagttaaatactaatactaaatgatgatcctctagggaaaagactttaggatataatcttctctgaaggacctgtagaggattgccctctatgtctcctacccagctgtgggaaaccaatccggaacagcaactggggtgttaacccaggaaatgtctccttaaaagctgactcatcttaggaatgagattctttttaagaaatgcaattaattattaatttcgctccactattagattaattcacttctttctcagatatgccattttctgaaaatatcttttaaaatgcattttggctttaaaaaatatgttcgtttacagaatctgcctttcctccagatctgcctaaattgcacttttaaaaaattaaaaatcaaagtgttttgcttccaactggtgatttggagattcaaagaatatctttcagtagacctactcttattcgattattcaagatttagttctccagatagcaatttgtaagtaagaaataaatcagtgagtgaatgtatagttgtacaaacttaaagttatggatgctcagctccaaatgtgtacattgtttgtatggttagcttgcaaaatataccataaccagtagctgaatcaactttcaaatatcattgggataataaattaacaatttcaaatgtaaaattatttaaacacataaataaatgccagtgtgttcaaccaagggagaaatggacacaagtgtattttttgttgcttccttaagtatttgcataatatctttacttaacacatgcccgctggttagcactttgaaattattttgaaaagcgtttgtcaactatctttcacttccctagccaagttttcattggcttctgttatttttctttgtctttctctagtcaattggttttcaaacctggctacacatttgaactacaattgcagagcttttaagcaaatattgatacccagattccgcctcagagaatctgattgaattggttgaaggtggagccaagcattgctcttttttaagttttccaaatgattctaatgtgcatccacagctgtgggccactctgtgaataagctgttaatgttctacccctttttgttttctttcagtatgtttgcatatctctttctctttagctgtgttttttttttttctcctgacatttttattggtgaggtcctataaatctatttttttcttttctcattttgcttttttcttttttacctcctatttactggccaagaagttctcattccaggtatatatatttgatttatattcttatcccatactattcatgctttatgtcagtgtaagagaacacaatatgatggtgaattatatttttgcctgttcttccaaagctaatttaatagtttacattaatttttggtataaatataaaaagatcaatagtagaaactaaggccattgtcaaaaacggatattaaggaaggatttattttaaatgtagttattattggctatgacaaaagacaataaaacagtttagaattttgagaccatacaggtctcaaggacatgtatatatcatatcaacagtatttttccctttcagaggtaaaggacggcaatgtgtttgccctaaattttaaactttaaaaacaaatttcaatagtatttaaagaacagagtggaagtgggaatattacatattgcaacaaataacatagcagaaacaagatatcactagcaacagaaaatagcaatagaaaaaaaagaaaaaaaaagaatcaatgctgctaagtattgtgatgatgttaatattgggaagaaaactagaaatcaagaaggtaatgcatagattcaaggatttgaaatgaaacagactgagcctagactaggagaaaaggaatctgagtgccaatgtagctgtgcttctaaatatggtcaagaggtttcctctccatgcggtttattttccccaactggcgaaaatactggttagattggcaaaaggagaaaaagaaaccttgcaattccttggccagatcctgcctacacatgtgttttattgaatctatctggtattataaataatttgaaatagatgattccatttaacaacagagggaaatatttactaacaatttggatttttcagcttcttcagggaagtcatttgatgtaacagtctcctagcagtgagaaggcgttttccattttaaaaagggcatgcattcactttctagttcctcaggcccaacatgaatggctttactgctttctcttacctactttgcccatgcaggaatttatgttcgtttcttctagaatttattatttaaggtcatgaccaaccattatataaattccatgaaaagaaaatgctcagagtacttgggaacatgtttagttaggcataataaaaacaacttatatggagaaaaataaatgtcacaagcagaaacaccaaggagacagagaaactaacaaacaggtgactagtcctcaaatagaggcttaagcccacaaaacgtgtcctattaaaccttatgcaagaatatgttggactctagaccagaagcgcaatgtcacttgttagaaatatgaattggaatttagtgaattattccagttcaagctctttcttgggaaaggaaaaggacaaaatgttctgtacaaccaaagtaagaataaagtatattctgaaaatcaggagattaaaattcccaaaagggagcttcttggcagaaaatggtcaaagttatttaaagagtgactttgaattcacccattgaataaaacaatattaaaccactatttaataattaagggaccatcatttctctaactttgctccagcacagagtttaatcacatgaagggctccctatatgcagtctccaaaaaaatgaaaacctaattcaattcttacagaaataactccagatttcataaggtttctctgaactaaaacccctgaaaattcatttatacaaccacacccagcatatgtttacatgtgctgtattttaatattgtaaacttgcttgcaagttacttttcagttgttctttattcatagatcttgggtaaaaccttctttaaagttctttccagaatgtcctttatcatttcaaatccttacaggaaagaagtgggatattaaaaaaagtaataaaaaaaaaaaatacttggcacattgaaacttgtggtatccagttaagtaggtattcatataaattttaaagaattattctactttaacaaattatatttctgatgtagattcagattttaggtatccaaagagctggtgcctatcaggaagttaataaaacaaagtatgtctgtgcttagtaaaagagcaagattttatttattgctgtattattaagaggaaacactattctgctccgaaataggtgtctgcacttgttacatcccattaaaatataaacaaagaatgtgttaatttcatcccagagggaaaaatattttcaggtaaagctgaactaaaacagtagcttctcaatcaaagataaatgtgtgaaccgacaatataaagcaagaatatgattttctttcttccaaattttctaactacctcaagttacaattgttcagtctacctcaagaagtaattcagatgtcaggttctatatggatttgccatgaaagttgtttataaggtaaaacgtaagcatgacattttgaaaatgaaagcagttagaattaaaattaatcgaaaatttaaaacgaacctaaaagtacagaaatagagttaagtaattatatttcactggtaggtgggtaattaatcctcacagttttggaaaactggtaaaacaaccattaatattaaggtgcgaatgaataatatatttttaggtgacaaatgaaacatgaattcccaaccttcagtgagttcatgtttctatacataaatgaaaatcagaaaaaagtaatcttcacagctacctatgtaaagaaatatcactatataatcaagtgttaatttcttggtgaaaaaaaatctttaaatgcttattcttatggatgcttttagttatcatcaaccctaaagataagaactcgtaagagctgggagtgacctagaactgagagacagatgagcaacaaattccaagcaccgcatttgtctagttgagaaacactaccctcatatttttaagcctcccaagctcttagccctctcagatctgcctaatcaagagtccattaagtcatcttggctgccatgaagatgaagctgacagcgcagttacttgagtcagtttaaagaggcctagggtggttgaagggaaggattaggaggcctggggtgctgaaaggtcatccaagttctagctcccaaagaccccagtagcacacagctgtcaggagcacgaagctacacttagaaacaacctttggaggaaagcagggattttgcagtccatcaaccattcatcaagcacaatttgcctttccacatcttattgtaccgttaatagaattgaagcattagagagtttgtgagttgatcacatagcaaagactttcctgagatttaggttcagttatctggtggtcttagtagaaattacttggcgtttaaatgtggttttggtgaaacctagattccctttgtgaatctttacagaaacttcgtctaacaggtatatcctcttaaggcaacgtgaaataaggccctttgcctctattttcataagaagaagaaagttttgaagtccgtgataacatattgggcataaataactgtcttctggaaggcattatgagttctataaactctccaggaattgcctggaatgttaacactaagtgatactattataggtacacaattgttgttgtttcagcaagggcatccttccaggatgagaattattcacttttcaggtacataattatttggcagttttattctatccttcctgttgtggtggaagatattatatcaccagcactaacgataacaataataacaatgatgaaaatttatgcctttatgtttgtcctgttccttttgatgtgagttaggatggcagggagagagaggaaggaggttcgtaatcatgttccaggatctataccaagcactgaacagacattattttagttacacaatcctgtaacacaggcattattattcctattttaaagatgatgtaataaaggctcagagagtttaaataacttgtcaggtaatagtggccataaaatgtaacctgccaagctcttgctcttaaccacttctccatgttgtttcctgcttgtttccactacagaaggaactggccaagaaacgcccctatttctctattttaccactttcatgtttagcacatcaatcataatttacaactcagtgagagtcaaagttgtatcttaattaagctgagaaaccaactgaagaaaaatccaaataaatgaggatcagaaaattttaaaaacaaagtattcttatacagctttgattctaatttccttcctttgtaggcaaggaaaatgctcttcagtgatacttggggtcttttctgagacaacaaaactcactagtagcagaacatgaaccagatcccaagactttcagctcagggttcaacctttttatcttcagtactcaaataaatcacagcatttttctcaactttacaaatgaaatcaccactacattcttttaaagagtaagttattcaaatgtgtttgatatatcatttaatcaaagcaggtttaatttccgaaatttttgaaagagcgcattttgtgaagtgaagaactctgagtgtatttgctttttcacatggcgaccacatgtatagatgaataactaagagactgttgcaactataccaagaaaaataaaagctaacatttggggccaaaattaagcattcattcataaatgtaaaccagttcaatcaagaccagctctgatgcttactcagaacaaagttactcacatttagtcacctgtagagaagcatggtgtgtacaatacactcaagttaattaacagccatttctgggaaccctgtccaaagaaatagttttctttatctcctatggtaggaattcttctgaaatagacaagacccagggctctcctctcaggcaaccactattttgaaatgtttttggggggactattattgctactcaatgtaaaatattaagtgagatgtcaatatttttcaacatatcattaggaattacagtttggaggccttaagttctatttttgaagcccttttagccagtactgactggaagccctgttgaaatcagaagtcttcatttcatatagggcacactctatttggagtgaatgtgatgaaatgtttccatgcaaattcctacaagcacttatgcctttatctttgtcctcataagccacattatactacctgttgtgtattttaaaaccctgtatgatttttaaaacttatctttcaattaatatgtatagattttctgtcaataatttcttattctaattatgtggtggctaactgaatgaaacatttatggttatgatatagaatattctaaatacaactacagacaatatggatcatagataaagtaaattattgttttatggctccagtggttcttcctggtaattaataaaaataaacagttattaaatatatacagaaaaacagcaatctatcaagcttatagaacaattaagaatatataataatctttaaatcaataaattaaaaggtaaacatttaatataaaatgaatatattaaaggataaagaagtagcatattatggaacatatagagattattttattttttgaaggttcaacctcttttaaatggttagtattggtgttcttttctctatgaaaacagattttagagaaaaatgatagtgtatttcacaaagccaaacacctctatattgttatcactccctatatgcatggcctctctaaagctgttaaataaaaaactaaagcacaagaatgatagttccaatttcactatacatttatgtttaaaaaatactggacacactgggcatttaaatagatgacatacagccaggaaactcattagactagctttaagtataataaaaaatattttttctgaatacaattaatcataagcatctacaaaaacacaagaccattgatgcctagtatgtaccataaatcaccatttccagacaggctctattttctttgaaagataaagaaatagtaagcagtttacacatcaaacgatgcctttggttcctgtaatttacatttaaacaacactgggttaaaaatagcagcatgaagatgattttaaagtaaggaaaatgacctggtagctaggttctatggtgcagataatgaaatgttttgtcagcttacctgataaaacagagccttatgaattacagcataacaaagcccagcatccaagatggttagggtataacgtgtcttcctctgcagctgttcagcttttttttttttttttttttttgaccacagaggtttacaaattagttacaattttttttctttctttctctgtggataagaaaacaaagagacctttccagaatcctctctgcttatgcctccgcttcctgttctgagattcactctatggtacccaacatatatcagaaaacagctttgaggaaaaaaaaaaagaaagaaaaaaaggccgatcgcactcaaagtggtaccgccatctactggcggacacacacaaaattgagccaaagaaaaaacattcccattatcctctgtcacagggaggaaagtaaacctgcatgaagcatgggtagtcatgaaagtgccccaagatgacagtagagaggtgatgtgagctaaaatttcccaacacactaaaaaaatgtaacttatctactacatataaatatgtatgagaccttttacatattcagttatttgcatctcactaatttttttaaaaaattttatcactttgttgttttgcatgattgtttcacacataaactactaaaattaaaagatatataaattactgtttgaaaaataggttttaagtttacattaccctttacgtgtagagttttgcgaaagctcccttgtggcctgctctagtcttttgaaaaccttttctcttcctgggatgaattattttcaaggtgaggaatttgactccttagagaacagtgtaaacacatactcactgatagtatttttgtgttgctttctggtaaaacatttgcaagaaatatctaaatgtagaaaaagattctatacatcagtgaacctagctaatattatttattaggatattgaagcacacaatacataaagaaaataaaaataatccatcagttattaagatatttctcaatccagatgtatgttattgtttcatctgacctttcatatctactttagagcaaacaactatgattttattacagtttatcatcaattaaataaatacattcatattgacaaaccaactgtatggatggacaaaaatgatgtcaagtattatattagacttgaaaagacacagagagaaacacacagtgcctgccataaaggagctcatacaaagaagaaaaacaaatatagctgataccatatttcccagccatgtgttcaagaactgttaattacatacttcgttttcaattgtggcccccattttgtctaatggcagaaaaataaatctagtcagttccaggtaaactaaattacttgaggcttttcccaatgattttgggtttgcattcaaccaatacatgattttcctcttctatctctccttctcatggagactgatctaagttcaggattgtgtacatatatagagaaagcagagtatgatcccgttccccctttctccacatgggcatccactgaggccaggagtcctgcatgatccatgaccatcattatccctcatatctactaacgtacccacaggcccactgggccccgggaatagcatccctccagctttgcatggcagcatcccctctttgtcacttcttcctctaaggaaacttttgttcctgtcctttaggaggagagaagaaaaagcagcatgttcccagggtcactcttggctgtaattggctgacttaagccttcctccctgccccaggcgcctcacacagccatctctgttctgctgtttgtaaaccatgggatgtaatgcactctgcaggcttcaggcaagcgaggcacaatctctgccacactagaatagtgtaaatagatggaaacttctcatcttttcatctttttagctaatcctagattggcaaactatgtaaggagcccttatcagagactcctttccttcttcataacttccttcacagactcaaaagagtttttgcttgggtctcttctttctgtaccaaaggtaagctggagccacttgttttcccttgaaatctttttgtcttatgtctaatgtatcagtttactagggctgctgtaacaaactgccatcctgaggggcttagacacagaaatcatttttttcacaattctggctactcagaggccagaatcaagttgtcagcatgatcatgcttcctctgaaacctgcaggggaaggatactttcttgcttctcttagcttttagtagccccagatgttccttggtttgtggcagcataactccagtctctgtctctgtctttacatggtgcactccctgcatcactgtctttacgtactcctccctctgtgcatgcctctgtgtcaaattttctcctctttataaggacaccagtcactttggtttagggtccgccctcataacctaattttaacctgattatctctgtaaataccctatttcaaaataagggcatattctgaattactcagaattagaactttttgagaggaggcacaattcaacccctaacacaagaatttaaatataaagcacaagaatttgacaattcttctgtccatctccaacgacaattccacttaggcaataatagccttggctatctggccagaattggggagggtaggggaagagcctcaggtaatgatgaagacatttaataatattagtaacaataacaaaactaacatttattggtactatgtgccagacattgttctaagcactttacatatgttcatttaaacttcacccatatgaggtattattatacacactttacaggtgaagaagcaatgaaatgtgagggaagtaaaacgtagttagtacctgatatggtttggctgtgtccccacccagatatcatcttgaattgtcagatatcatcttgaattgtagctcccataatccctacatgtcatggggggcacctggtgggaggtaattgaataatgggggcgagttttgcccatgctgttctagtgataatgaataagtctcatgagagctgatggttttataaagaggaacgtgtcccctgcacacgttctcttgcctgccaccatgtaagatgtgcctttgctcttccttcacattccaccatgattgtgaggcctccccagccatgtggaacagtgagtccattaaacctctttttctttatacattacccagtctcaggtgtttctttatggcagtgtgaaaatggactaatacagcacctcacatctcatcccaacacaagcagttgtaatcgggtaagatcactcataatacaagcatttgtcagatgctgaaagaacacaaaggaaatcagcttccactaatttagatcattgatactcacacgtttagtgtaaatttataacatttcattgtttcattgtgtacactttctgtcatgaaagattcctattttctaggcacttttaactttttcttctctactcatgaacaaactagccctgctcactgtctctcaactaaatgttcattaagtgccagtccctactgctccacactcattttaccattactagtaatatttaatagaatattaattctatcactcagttttgggtatccagatgctccaaagagttttctgatgacgtcttctgaggaactgatttttcttaggttatactgggccacattttaatgagaactggtttgaacaaaataagaaaagtaaatgataattattgtggcgatattcctcatagaaaaaaagaaaaataaataaatgccagcaaagagagctttactctttgttgctgaagaaactaatttatcttgtcattaaaagtagaaaattatataaaggaagagtgaccagaggtgtggtatcatttgctatgattctaacttcattgtgactttcgtactttttttctgtagacagaacgacaggtagtttgcaaatcttcagtgagtggattctgagagggttaaggtgaaatagtcattgattaccaacataaggtctctggagccttagtggtgtctgaccatatgttgtaaacatatcatgcataaatcacagaaattcacactagaagagagctaatagcctccagctgggactcataggttgctctgtgcatgtctactgtttctgattactcaaggtcttcagagcctttgtgggtaggagctataatgctggtttctatttgattttataagcttcatagccaaagaatactttttgtaacatccatagatctaccaaaagattcccagactctggcccaattttcttgtttcctagaccattatttcccagacagtgattggaggaatactactgttctgctggactaatgaagtgccactccacatccaccagaatggctacaacttaaaaaggcttatcatactgtttgtgagaatctagagctacagaagctgtcagagacttcaggtaggaatgtaaactagtataaccacttagaaaaatggtttggcattaccaactgaacgtgagtctactctgtgagccagcaagtgccactcttaggcatatacctgacagaaatgtgtgtgtgtacaagaatgcctgtggaagcattattgaaaacaggtcaaacaagaaagaacagaaatatcttgaacaggagaatggatataagaattgtggtgtattctgaatctatagtatcatactaacatgaaaatgaaaaactgcagctacccacagaaatatgctggatttcacaaatctgaggctgaggacgaagaaggtatgtaagcaaaacattattaaatttaaaaacaggactgggcgtggtggctcaaacctataatctcaacatttttgaaggctgaagtgggaggatcacttgagcctccaggagttcaagaccagcctgggaaaaatggcaagaccctgtctctacaaaaaaaatgaaaaaaataaaaattagtttggtatggtggtgtgcacctgtccagctactcaggaggcagagatgggagaatcacttgagccagggagttccaggccacagtgagctatgatcataccagttcactgcagcctggacaacaaagccagattcttgaaagataaaaataaaaacaggttaaataaactctaatattggaagtaagagcattggtttctcttgagataagatagagcttttggcaagattggttgagataagggctattctgggtactagtaagatcctatttcttaatctgggtagtagttatataggaattcacttcctggtaattcattgagccaaacatttatgcacttttttgcatgtggattgtacttcaaatttcaaagtaacaggcgagttttttgacagttgtcacctctgaggaagaaaaaagtaatgaaaggtgaaataagtggaatttccaggatttgatctgtatatatctgttctgtttgaactttgtatgcttattagtgtaataagaagtgcattattagtgtaattttcaaaaaataaatttaaggcattaaaagtacagtaaagatgtaccttcatactatttgtacttatttacctctaagttattctctttttaagttgagtctaggatggcattcatcattattttaaattgagtctgtcctggaattcctcttatgccagaaggcctattcaagctgatactcagcttctatgcttctgtcttcagtcatcttagtaacaaagttaactaccactgtgtttgcatgtatgttaaataaacaaaatttacatgtcatctacaatttcacacctctatagtctagtttagatttgtatctcctgattgtaaatactgagactaactcacttttgcccactgcctaatatattatgccataaattacacaatgaaatacatgacctgacaaaagataataaaacacttttacctttgttacatcttcttcctttaaaacatctaaacaagttgagaagggtagataattttcagatttttcaggtgagaaaaattggtacagaaatgtgaaaattacatgatggttaaaaattggccaaataaaacaggataattgccagtttaaaatatatgcattagacaatatgattttatatcaagggctcatttcagatataataactgtcaaatattatttagaaggtgtgcttgattcagcaatattaaaaaattttaacctgtattataaaagaacaatagagtaaaacatactttccaagtggcttccagtatagattaccttctgtgctgtagttgcacagctatgtaatagcaatttttcaaaggtgttgggtcactatgcctttttagttgccattcctttggttagaaattactttcctttattgccattctcacccctatttccctccaacatctggaaaactactatttattcttgaaaatgcaattagggtaatatacttgatagcttgtcctgatctttccaagtgtaaggtatcaacttaactgtactccctgtcatactgctatacacatcattaatggaggaggtgaaaatgattcatccattaaaatgactgaatattcttaggtgcttgtaaacgcccattatttaccatggagtttcatcagaaaagacaggtgtgaagcagataagggcaggtactgggagatacacgtctctctgcttctctcaggaatgatggctggataatctgtttacccatatgcacacacactgtctactctagggctttgtgactgagcaacaggcttttttttccctatacattctttgttagtatgacactgtcagatcatctaagctaaagggaaaaatgtatttttttttcatgagttcctaacaccttttgctaattataagcaagcacgcaggcatttcccttatataatttctcattttatcttctaaaatttggtacatgtgagtagccaactatgtccaaatctttatatgattaggccaatatgaaaagaattaaaattttaatttaatatgctctcttttattttggaaaatatttacttgcatcatatttccagcttatattatatttttattggttgtttagcattctttaatactaaaacaataaaataggaaatcatgcttagaccacgtcttctaaatttcgaagacgtaatatgttgaaaagctttatttcatgtataatacattttcatttagtttatcaattttaaaagataatttcaactaaaaaaatccttcacagtttataagtgcactttattagtggagaaaatcaagggttttaaaacatcagagtaatcatgatggaaattattatacgttcatatttcagaaaataactactctttttttaattttacaaaggtaaattgacaaatgcaactttcccataagctctgaacaaaaagaggggctgttaagataagtatgaacatgggggaatcaggggaactgggaggtgctaatgaagaaaaagctagaaaagaatcaaagaactacattatttaaaaacttcatctattaggaagtggctaagaagaaaaatatggagagagatggaatgttactgtcataaacctgggagaagatgatcttatgcaaaagttaatgtaagcggaaatggacacaagtatatggatttagaagatattatggtttatgttgtagatatgaattttaatctatatctatgtgtgtgaaaaataatagatcaacagtaacttcaacttcagtaattactatagggcacctccactcaatgtatcaaagttccagagtcttgtgtaggacccaggtataaaggtagaggtgaggggctcaatcttttgtcatatatttgcataggttacaaggattcatttatttaacaaatattattgagcatctactctttgccagcaactcttcttggtgcttgtaatgtatcagtgaacaaaacaaagatccctgccttcatgaagcacatgctttgggagtggcacaaatgtggacaataaacaataatattaatgaattagtaagtgatattaaatgtcagaacgtgattagtgctaagtaaacaagtaaaaattaaaatctggttgaagagatcagaatgtttgatggagtggtggattgagatttaagttagttgatcatacggaaggccttattaatatgataatactttaagcctatgaccagtgtagtaagcctagttcagagtccacaacctaggcttataggagatgctacatttccttatattctacaaatatattttatttagattttggtgccccattcgaacgaagtggcaagaaatccaaattatttattttagaacttatgaccccctctcccatcaccagtgattaagtttcccaagtcttacctaatgacattagtcttctctccaaacaatttacttttcagtttttcattgatatgtttacctctctcatacatccgtgtaagatatgccgtgattccccatgaagttggaaactggtggctaaatcccttctacttacgcacatcaggaagcagtcctttggtgagatagtattatctctctagaggtcttccaggaaacaggctctctgctactcagattcctacaggcctatttcttttttcattatttggaagatttttctgaactggagcatcccctcctgcctccaaccacttcttcaagacacacagcttctacccccaaaatccattttttaaaatagactttgagagttcattaaatccttaatttaaatctatggcaactaaaaaactatatccctttcatcattgtcatgggtgtctgtatcttcaaacccttacaagagcttggggaaattatttatcttctattctgcaagtataccaaagaatagaacataatagttgttttcatactaagtatattttcatagtaaaagagtgaaaattcaaattccatcacaatattgaggaggtatcattaaacctgattttgaaactggatagaacagtgagagagaaggaggaataatggtgaccctcaggcttttgacttgaagaaaatagtatctctgtatgctactcatgaagataagtaaacattagctagaccaggtttgtgtctttgaagatgagtcatttagttcagggatttctgtgtttaaactacttgtgggacatccaaatagaaatgatcagtgacatttagaccaatttgtagtttataaaagagatcccaggtgaagagcttgcatcagagaagaaatcatttttccgtttctgttctccctaccattgctccagattctgtttccttcaatttctgctcaaaacttatcctttctattattttatattattgtatctccttccaaccttcaatcattatttcaatcttcaattgtggcataatgcctctgactatgagccgttattaaaaagaaaacaaaataagggaaggaaggagagtaggaagaaaggaaggaggcaggaagagaggaacggcaggagagaggaaaggaagctcttcactaacgtttgacaatttgcgccagcagcactctgtttagtcttgtgccttcagggatgacttttaatatatgattcctcaatatttttccagccttaattttaatcttctgattgtcagtagtgggtcttcaaataataagcatctgtctggctgccatgccaccaccaccaactcaacataatcttaagctgaactggtcatcatgcttgacaaataataaaaacacaaccttgtcttattcttaataataagtattaaatacattccattgctcaggcatggttttattcatgactcccaccagccactttggatcatccatggtccattgctcacttctgcctctgtgcctttacaccattgactttgcccagcctgaaatctatctttttgcaaggtgcagatcaagtttgacatcctctcttacctccattacctagaactaagcacttctgatcaataacatgtctgagagccaatggatttttttcaccatgaacttaacataaactacttttattgatttatctacccttttatactttgggtgttaccttaagaaggacattggaagtttttctatctagtgtgatcttgttgtatgtcttttttacgttcacagtgcctgaaagaaatttttcttccactgaaggcttgatgctatgaaatggagtagctcactgttttttctccaatccatcaaattcctttctctaagtatgaaactcggtaaccactgacattcctattccaaattcaccagctatgtgattttggtcaagttacttgaccacttgaaaccttaatttctgcatctttaaaataaagacaacaccaattcttaccttgtagacttctgagcattaaatgaaaaaaaaatccatgtgaagcatttagcagtgccagtcacagtgtccaacaaatatttagtcataatattcatatggtcatatgtttctaatttaactacagcactctaattatacaacaatggaaaacctagagatgtcttcatttcctcctgctttctcatgtttcttatctgatcaactggtgatcatacaaagtctctaatcagcaccatagcactgctaccaccggaatgaaatctttatgacctctcactagagatactgcaatcattttctgacctaagtgattccccactgatgtcaagcctgcttcctagagcatcattttcaatatcccattctcctaaacaaaatctttaagaagccccagtctcctgctgtagtcaataaaatcaatattgaactcctgaacctaatattcatgactttccacaaaatatccactatccaattctaacaaaccaactagaaaaaggcattttttaggtaagagaggaattttgaatatggactagttattaaataaaaacaaggaattatatttattttgctatgagtaataatggtattgtgttatgttaaaaatgagggtatattttggaaatgtatcctgtataacataaggatgcaatgttataaaaatctgggttttgttttaaaataattcagaaagagaaaaacaaatagatgaatcaagtttggtaaaattttggtaatgagtgcatatgggataagtccattatactgttctttctgcttttgaatatgcttgtttttcacaatacaataagtaataaaatattataaattatataatccattgcatatgctacctgtttcccgtacttctaagagctgtttttccctactctaaatccaaccacattcttttattgctcttttgtcatttattatctgtagatgtgtattataattgttaaatattttagaccatctgttagattataaactttatgaaagctttaatatattacttgtccttgtatcccaaaattttctaatgtattttctggtacacaatagcttacatagtaaataattgtgtaaacaaatgggcaagaccataaaataaattacaaacatctctgaaagaaagggttatttctttgataacaaaactaatgcattttctaatgttgtagcatttctagcctgagacctaattaagcaactgaaaaagaagtggtaaaattaagagccactgtcagcaggagtactcataccataaatttttaattgctgaattttgagacaaataatgacttttaaaaagagtagacagagtttatgaataatgattctcaaaatctacccaaataaaagggtagcaataacacaattgttaaggggacagaaatcccgtagggaaatgttaattgtgttatcatgttttaagcctggagaacataaagacttagaagtaactttaaacatacaaagtttgtttctattggagaaaaaccataacctattatagacatgctcaactgtatggacagattcaaacaattcttttctctccccttataaataacactaagtatttgtaacatcttttggagggcaactgtatatttgtttttcttgcaatagattagcaatacaagttttgttttttttttttaatctctctctggacaaaaaaaaaaaaaaaatagtgcctttactttccttataaaatgtttctctagctggagatataatttgccccaaattggcaacgtgtagtcttacagaaatttctttccctctatcagccagcaagacaggtggccctagctttggtccccattgctctgtgtcactgcacacctggacctaattgttcagtctcaagtggagcaggtgagatgagggtgggctgttgtgcaggaaatgaatggacttaacacagtggtttgcttcggcaccaggcagtcttatttgagtgggtgggcgggcttaggctatgggaggctcgggagggagccacatagaaggacagataatgcaaacacagactttagattccaaaataattcctggcattaaggagggcctggcagcctgcttacatagtagttaaatgctggttaacaccaaggcaggtgctctgcatgggctggacaattgcaagcacttggataaatattagttgttgatttattatcactgttttaagagtgtcggtttcccagccttcaggtagattctactaatttattctatctatccccctgtttaataatttattgagttttaagaacaccgcagaggaagcagccattgttaaaggaaggtttcaggtcacttgacctgggtagatttgtaaacataaataaatgtgtattgcctgtttagctctatttcttaggggcggaagcgtccagataagaatttgtcattcgcgtatgctatttaattattatgacagatcaaagagacatgctggatgtgttttttgtgaaaaaaaatcacataaactcaatgtaagcaggggaattgagacagggaacctcatctttaaacttcaaggaaaaatttgaggctgtaactgtgatgtataaaaaatattaaagctcaggttattcacttagttaacaagtatttactgaaagcttagggtgttccctgcattctttaggatgatgggacataagtgagaataaaatacagtgcctgatcgcaacaagctcacagtctatagaaggggactgacaacacaaacaacatggcagttttaacatggctgcttatctgaagcctgaggtatgcacaggtgctttaggatacctgactgcaaccgctcttcaaagaaaaattatttcttctcttgatgtgaaatagataaagcttcaaagacgggatagaaggctttaagacatgtagatgttctgaagtaaatgactttacacagaatgatttatgcgaagtgcattaattggtactgctagaaagctaatttaaagaaaatcatatggctatattgacacgatgtgaattttagagctctttagattgtaagtgacagaacttcagttcaaaaggattcaaaacaaaggggaaataaaagtctcatggagctaaaaattgtaggagctagaggtactttgggcacgtctgggcccaagtcttcaaatgatggcatctggaatctgttcctctcatcattggcccagttttcttctatgcttccttccttgttagagaggttaccggcacatgacatactcctgcaagtcctggctgacattcagcaagattcaagaacagtgagaatgaatatattttattaccaaatgcttcatcagaaatcccagaaaaacctctaatagcttctgattgggtctggtgtccaaccttggctaatcccaggagccaggacaatgtgagtctctaaataaccaaacctgggtcacattaccatcttttgagaagggaatcaagtcagcccacccaaatcatgttgactgagagcagaagagaggtagttacctgtaggaaattggagagctcttacaaaaagtgagcatggttgctggactttttaattacattttttttaattttaagataattgtagactcatgggtttttactgttaaacatttttcttagatacttaaaaattaaaatctattcactcaaaattactaagctattctctcaaagcctgttttatttttttacatgggatacatatgctgaatgtgcaggtttgttacataggtatacatgtgcattggtggtttgttgcacccatcaactcatcatctaggttttaagccctgcatgcatttgtcctaatgctctccctccactttccccacactccctgacaggccccggtgtgtgatgttcccctccctgtgtccatgtgttctcattgttcaacttcaacttatgagtgaggacatgtggtgtttggttttctgtttctgtgttagtttgctgaggatgatggcttccagcttcatccatgtcctagcaaaggacacaactcattctttttttgtgactgtgtagtattccatggtgtatatgtgtcacattttctttatccagtctatcattgatgggcatttgggttggtcccaagtctttactattgtaaaaagttctgcaataaacacatgtgtgcatgtgtctttagagtagaatgatgtataatcctttgggtatatactcagtaatgggattgctgggtcaaatggtatttctggttctagatccttgaggaatcaccacactgtcttccacaatggttgaactgatttacactcctaccaacagtgtaaaagcattcctatttctccatatcctctccagcatctgttgtttccagactttttaatgatcaccattctaactggcatgagatggtatctcattgtggttttaatttgaatttctctaatgaccagtgatgatgagctttttttcatatgtttgttggccacataaatgacttcttttgagaagtgcctgttcatatccttcacccactttttgatggggttgtgtttttttttgtaaatttgtttaagttccttgtagattccagatattagacctttgtcatatggatagattgcaaaaattttctcccattctgtaggttgcctgtacaatctgatgatcgtttcttttgctgaacagaagttctttagtttaattaaatcccatttgtcaattttggcttttgttgcaattgattttggcattttagtcacgaagtctttgcccatgcctatgtcctgaatggtattgcctaggttttcttctaggtttttatggttttaggttttacatttaagtctttaatccatcttgaggttatttttgtataaggtgtaagaaaggggtccagtttctgttttctgatactaaaaagctatgtgatattaggcaaatcacttgtgttctcatcttcaaatgtaatttttctggttctattttatagggttgttcacaaggttaatacaatgacaatatctttttaaaatggaaattacgactaaaatattgtattgtagcatttctttacaagcatatatttttaaacgtgtttttactcacatttgtataaagttttatttcttttatattatctctacctcttatgtacacagagaaacacttgggtttaacttctaatatgataatttcgcctcttacactttcaagaaaattaagatttggattttccatactccttcattggttcttcattgcttatttgtagattaattagcaagcattatttatattgtcactgacagacagcatgatataagtgatgataactgtcaatcatgcagcttactgggtttttttttttttttgagtgagtggcatgaacatttctcttcactaattagaaatttattctatacaactcttccttagggttgaccagtcctgcctttcactaagagacttctcacaatgaaagggctgcctggggcgcactggaaagcttagtcagcagcggaattaccttcttcctgaaggctgactgaagaagggaatgtggcaatgagcaagaggcttcacaaatgacagctagtcatttgtgctgggcgtagagtcttcttgcctgcattctaagatggtggggatgacaggaagcaggtctgcatcatcagctcagcacactggcatggaaccaggaatggactgggaggcaagacttttgcttaatgtctgagtttccttaaatctttttagaacaaagcaggacaaaaattttaaaataaactagtatatgaccttgaatacattaaacaaaactattctacaatttttctagcttagaacactcttttagcagtcattttaaaaaacaaattaagattaaggaagcagtttatatttcttaaattagtttattaattgttttttcctgagtagtaaagtcttaagcaatgcgtagacaatttggtgagcaggtaacagctataatcataaaccagaattctcacctcctaaggaccactgccagccctccagagtctgacataagtaaaccacacaattaagcaaattcttgatcaccccagtgatatagatagaaaagctccgatagaaaggaaagtctattctgattatgatgggttggtgacatggagaaggattaatgattcagactgtagacaattttggctatatagttaatacatatatacacaaaaatatgtatatttatatattgaaagtgaataatagcttatatatatataaatatagagagtaatatttaaatgacatgtagaaacttaagtcctacaattctatgtgtcactgcaaaatagcaagctaactgttgaagaagaaagtattgacagactccattccatagattataaacctgaaggaaataaatgaggtgccaaggcaagcttctctcccacgtctcttgcataagttgtgggcattgcattttcataacacatctttacaccattttaaatatgaatgtcaattgcaggaaagattaaaggcattgtcctaagagcttaattcacagggcgtgtcctaagtattagcaaaagcgaagtccagtatgggtaagagcaaataaaatgagattgctgtttaataagccatatttgtaattgaacacaagactttcaagtgctagaactaaaactgctttctgagagctatcatatatcagatggtttccctgagcacttgcaaattaaaagatttttaaagatatagtgctattccatttatgtgttttacacagagttagattgatcacattaataatcaataacatttggaaagaaatgatgtttgttaagccagcctcaagttatgatttatttatttttatgtagggactttagttggctgggaaaatgactgctcagttctcgtttgcctgccaaaaagcagacattttagaaacagtgatgtaaaagtcagtaagaattatgtggcttcaagtggcagaatacctacctacaaccggcttgagcatagagagagattattggctcatgatacttgaaagtccagaggctggtatggcttcagacaggtccgcacccagggaatcaaatgaaatcatttggatccatcctctcttccagcttcacattgtacccagatgtgaagaatttagaggcttttctttcctcactcctttcctttcctcactaatggctttagtcatagatacactctcctgtcttgtgccacagtggcctccaggagctccatgtttacatccttacagttctagttcaattgaaaacagagtctgcctttctttaaacagtttccaagctctgatattccaatccactggctctgattgggattcatgtccggtcttgaaccatgtttggtgaccaaaggactacagagtcttaaccaagtctaactacattacgcatttgtgccagaagctaaaggaagtgtcagcatcacctgggcagtagatattggatgcttgagcaagaacattctctagagaaaaattgtggtgcaggcataaggagatgaggggtgtgtgtacagggtttcaagtatagcaagtgtccagtatagatttttgttaccactggtcaatcacagaagaggatagtaaagtgaagggaggttgtcttatggtgacatctaaaagccactaaaatgacattccctggaaatagtttcttgaagaattatgtcttgtagggaatatgtttgtggaagctgaacacacaaataagtggacggcccctctccatgacccaaattcagatacgtctctagcaaaagttctatcacagatactcttactattagtcaatgtcttcctatatcttgctacacctctgattaaaaacaaataaacaaaaaaacttgtctgtatgtagaactatcaatttttagggtgatttcccatttattttttcatctgaaatgttctctaagtgctttctattttattagctaaactaattttactttgtttaaatttcaaaatgatatatagagagtcaaacagaatggcttctaaaatattttagtttgagacttactctgtctgagcagccctaactacatttttctctttaagatacagttttattaaaccctattcacttcatactaaatttaatactagattattatatccaaggaaagaaaaatacttaactctactaaccattctgttttattttctttttgtgagatttgactcttttttccagcatattgaggtattttgaggtcaagtttatcatgaaataaacttccctattcctcaacccatcacattattatgtagattgggttaaagtgaggctgcaatgatttcctcatcctatgcagtagtttcctctacctgaaggtttcttaagcaaaatatctactcattcctagagccagacttgagaggcatgtaccttagagttcttgaagccaggatttatgattcagttctaagctgaagatggcgaatattaattgcgaaaggatacaggaagaaaaccctccagattcttcacatctgggtacaatgtaacataaaaggaggagcaaaggtattaaatttgggtaagaaggaaagaatgttaaggacagtctaaagattgtgttttacctgtgtatatacacttttagccaaattgccagaaataacaaaaggagtgataaatttcctgtttaatcctgactactgcaaattgactgcacccctactattcctcagaaaccaatttctctgggttcactaatgacctaaaactgccaaatacaagaacatattcctgtccttatgttagttgttatttctgttttattttgtaatcttgactcctaattctatgtaattccactccctgatggctttgatgacaacattcatcttaatgacttttctcctatttctgtggtcctgtttctagtctctcatcatccaagtcagaaactgagagtcagactaaagtcatccttcatacatgttccttctccaacacattcactttgatttttaacagcttacgttaagagaacttgcatactgtaaagctcactcatctaaagtgtatgatgcagtaatttttagtgtagtcacagattcatgtaactatcaccacaattaattttaatacactttatcacccccaacaggaagctccatattcattagcagtcactttccatttccccccaactttccatccctgcatccccagccctaggaaaccactaatccactttctgtctttatcaatttgcctattctaaatgttccatatgaatggaattatatatgatgtggtcttttgtgactggcttttttcccttagcataataaactttgaggttcaaccataatataggacgtatcagtatttcattcattttatcaaatagtatttcattatatagctatatcaaattttatctatccattcatcagttgatggttacttaggttgttttcacttttgggggattatcaataatcctactagtagcattcatgtataagtttttgtatgaatgtatgtttttatttctcttgggtatatacctaggagtagaattgctggatatcttttataagggtgctaatcccatttgtgagggcttcgcccttatgacctaaccatctaccaaaggccctgcctcctaataccatcacctttgagattaagatttcaaaatgtgaattttgaggagacacaaatattcagtctatggcacatagtcatatgtattagttcattttcacgttgccaaaaagaaatatctaagactaggtaatctatttttctttaaagaaaaaagaggtttaattggctcacagttctataggctgtacaggaagcatggcagcaactgcttctggggaggccatggggaacttacaatcacggcagaaggcaaagccagagctagcatcttcacaacatggccagggtatgaggaagagagatgggggaggtgccacatacttttaaacaaccagaccttgtgataactctgtcacgaggacagcaccaaagggatagtgataaaccagtagaaactgaccccctaatccaatcacctcccaccaggacccacctccaacattggggattacaactggacaccagatttgggcaggaacacagatccaaactgtatcagcatacctactcttctttccagtttgtgacctctgtcatagtttaggacctcattagttttctttttctttttctttttttttttttgagacagagtctcactatgtcgcctagtctggagtgcaatggtgtgatctcagctcactgcaacctccgcctcccaagttcaagctattctcctgcctcagcctgctgagtagctggtattataggcatgcaccaccatgcctggctaatttttgtatttttagtacagacgggttttaccatgtttgccaggctggtcttggactcctgacctcagatgatccacccgcctcagcctcccaaagtgctgggattacaggtgtgagccactgtgcctagcctaggacctcattagttttcactgggattggggaataaatatttatagacatagtgtgtgtgtgtgttttagtatatatgtatgtaagtgtatgtgggtgtttgtgtgtagtctcttctgtatatacccatatatatattgctgtttgtgtggtggtaaacttcccttttcaaggaagggattactttataaagagtcaaaagtttaaaatgaaaataatctgaaaagcaattcctcactttctcacaaactctgagactgagtccttagaaatcagttttttgctttgttctttaagaagtaaatgataggaagagggctgagtaattgaaatgagggaagtgatcttattaaatagattttgaatcccaattatacttaaattgtgtttctgtagccaaaaatgtaataaacacgaataaatcttctttatcatcatttataataccatctgtacagataatctaataacctctatggaatgtctgctctgttctaggcactctacatttctttaacttaatttcacaaccttacaaggtagttaaagatgagtatcacagaggcattaactgatactagaaaggttaagtgaagtgcccaaggtcacagagctagtttggatctgtgttcccacccaaatcttgtgccatattttaacgaggatttcacccaaggattctcttgctctaaacccacattctttcttaggccaagtacacatcaaagcattattctattatttaagtgtcagaataatgaaaaaaaaacaaaccagcatttcttagagaagaaaggtaatgtgggctgaggctgtcaaggacaggatcagaaagaggtgacttttaagccagcccttaggaggagatggagagagggtagagatgttttgccataaagaatagcatggctatcattgataggagctgaattatgagaacacatggacacagtgtggggaacaacacacactggagcctgctggaagtgggtaggatgaggggaaggagagcatcagggaaaatagctaatggatggtgaacttaatacctaggttatgggatgatctgtgcagcaaagcatcatgacacacatttacctatgcaacaaacctgtacatcttgcacatgtaccctgagattaaaattaaagttgaagaaaaaaaaacgaatagtatggcataaggaaataaatagtctcatagagcattcagtctgggcctcataagagattgctttttttttgcttcatctctttgggacaaatgttcctgcctgatttaatagaaatggtatattctctgcagatacctttgagccatactctgagatgcagagatattttatgttggaaccagctctgtcagcatgcttgaataggcagaatttttcctctaagctatctgaaatggagctgagttaccaataagcaaatcataacacacaagaatggaaaagacgaacatttattccatccaactagtgcttacattaatctgattaattaaagcatgaatgaaaactatttacttgtgagtgttgtgaaagagcagcattaaattagcccttcaccctagcagacacactgtgattgttggctctcattctgtttttagcctcattgctttcttgtatttcctactagtggaagctgggccagtgcatagccagtgaaggctttctcttatttcactggtgaccagactcaaaaaaagcaaggtttttatattgaaattcgtcaagaaacttgccatcagacgagtacatatgcatgaccacaaggtcactacccaaatggaggcaagaaaatgagagttatcttttgcctttttgaagatttttccagaggggtttagttgccagttgggttttatctaatttagtttcctctaaatatttttataaacagcaagtttatttccagttttctattgtttgcattaactagggggtttaataatttctcacatttatcagtggaaataaactatttttattgaatatattctagcaaaagatgctttgaagttttttttggaaatatttaagaacctaaagaatgtgaccaaaaattcctcataaaatagccttttgctcagggtcttatgttggcttggattctggctttgaccttgcaatctctgtctatagtgaccacattaaatgttacttggacttaataagatcttctgccttcaaagatgaagcaaaaagtgttgatagtttttaaaataaagaaatgaaagcaatagggtgcgagaagtgtcttaaaattgaaataaactcaaaataaaattgaatttgtaagtaactggttatgacatggagtgccataaaactgaaaatatttgactgtagagatttcttggtaaagaagagaaatatttggagtcataattttaaatataatgtcaagttctgtttaaataaacaaagtttatttaaaatttagacaagatagaaggccagagaaagaaagggtagctttatttagtaagtcaaaatatatgttatagcatttatcaaattcataagatatttgaccaagtgataaatatgaagcttatgttctgaaagggtagacaaaaaatgcaaccagctttgtggtagacagttgtttaagtatattgataatatcctttttaaaatttgtccacctgaagcctctcttgatcacctccttgcctccaaccttggaaattttgttagctctgcccctaatacttggctctctctattacatctttaatacttggctgtattaggaagtatattccactcagtggtaatcttggttttgtctccttcttatctattagaaggtgagcaacttgaagcagaggcttcgctttgtctttgtgtctctagcaccttgccaaaggcctgctaataaataattatcatattgaatgggtctacacaatgttctcagtatgctcagacatctgtgatcacctttacttacagagacagggcagatttgaaatatttgcccctatctttgaagctaatacctggaggtgcaaactaggtggagcagagcccaggagctatttctaggaagaaagcatttctctgatcctttgcttctgcccacagtgttaataacattttgttctgcacccacactctcaatttcattccagctgggactcttctgattagcatcctgctctttgccttcccagagacagaataaaaggagaaggagccacaataacagaattagacaaattgtaacggtgtcttatgtcaatgtcaaagggagaaaatataaacaagttcaaaacttgtcttgggataggttttttttttttttttttttttttgaggtggagtttcgctcttgttgcccaggctggagtgcagtggcatgacctcggctcactgcaacctccgcctcccatgttcaagcgattcttctacttcagcctcccgagtagctgggattacaggcacccgccaccatgcccggctaatttttgcattttcagtggaggctgggtttcaccatgttggttaggctgggtttcaccaggttgggtttcaccaagttggtttcaccatgttggttgagataagtctcgaactccttacctcaggtgatcctcctgcctcggcctccaaagtgctgggattataggcatgagccaccgtgcctggcctcaggagagttattaagcatagcctgccaataacttcaaaataactttgtattagtttgtgatttcagaaacaccttcacaaaccttttttttttaaatttgattgatccttatagcaacattgtcagtttgggtgggcgagggagttataatgctgtatccacacgaaggaatctgctttagttggttggtaaagtcttcacaggacctggaatttacaagattggtagcttgatatacagaagatattggaaagggaaaaatgttaccaatagagataacaatgtgagaatgaaacagaggcataaaaatgtatgttataattcagaatactcagtagtacagaaactaagggtacatggtggagaggggatggaacctacaaaggtaaagcagccaggaatcacatcagaaaggatcttgcttgccatgctaaggggcttggacttcatccttggacagtagtgagtcactgaatgctgtttaacagaagacagctgtgactatttctgtgcttcataaaaattattcagaagcattgttgctcatgattagaacagaagcatgccctttttttctgcttcaaaatatattctattcacacttgaaattcaattgcatgatcctacccaaggtgaagggcaattaccaacctgttcattgtaaatctcactcttctccaaagaaagagtctccagatgaaagtgagggggacctttgctattacagggataatctcagatacatttaaattttattaaaaatcatttgcaactattagcccattatcattattagtaaattaaatatgatattctttatgtttaaaacactgtgtctgatcagaaagaaatgacagtggaagaaggaaggcagaagaacaatcagagcactaggtggagaaccaggagagagacctgacttattggggcatggcagagaacctgagctttgttcctgtaaccacaactgctactgaatttatcccaacagcaaaactcccactcttatattttaagccttctctgctggtaccctgttctaaacttacaaggctggatgactcctaacaagtgttaaaaatacttgactgtagcctagggacatattttttttaatgtgtgggtaaagagttttccttcttcaacaagttcaattgaagaaagcttcttatgcctatttcaaaacacacgcacaaatttaggagcagtgagatgaattctcttgcagctattttgatttgagttgtttcccccttggaatttgtggatagaaactgttatattaggctccacatcattagttatatttcaacttgaactttaaaagtagggctggagacctgaggtaatgattacccccaagaatccttttatccttttaagggcagatgaggtagatgagtccctacttttgtaactagaagaacttgataggctaatgtttctgatgatatcttctgttaaaaaaaaaaactaaccctgagatactctttagaaagagttctgttgtccaatacttattttaaattattcttataaaaagctctctattaaagaatcataatacacatcagtgatttaaatatcttaagaaatccttcagtaacaaaattaatctgtttaacattttattgcttgccctctacttcctatcttcctataatgggtcagaatgtggacatgaatgtcagccaacctcaaatatgaggccgaggaaaatgccctaagagcgaacacaatagcaaaatgggtccctgtccacctcgcaaagcatagcctgcctttcaccctagacttttatggggagatcattgtttgtcctgttttagtgagggtttttaagtttcatttgttgtaatatctcagcttatacactcttatactcaacatacttaaactattttgaccacagaaattgtcttaacggttttattggaaaatctgctatcaataacatcttgaagactacatttctaaaaaagatattttaggcaacactgggcataccaaaggattctttaattctgatttcttgattaacaatccttgctctgtgcatggcttgttccatgattttcctattctctatttcctggttggcacttctttaattgctgacacaggacccatatttcacctatctgatggctcctgagaaccctccactgggtcctctggacctggccaggatacaggggataaaagagtatgtcgaagattacttgagaagtctggggcaccattatgcagcaggtgaacatcactcttgcttaatactgtattggttgtaagtcagtcatatggtcctattaactgccagggaactgggagatgtagtttagttgcttgttagggaggaaggtgaaatcattcgatgaacacagagagttgtctctgctctccttgaaaaatatcagagattcccatctttaattcaactctttctagttgttttctcttttggaaatacattaatgcttcttcaaggagccaggcaaagcaatcagatcactcactgtaatccttatgaggaagtgtatatccttttcatggtcaaggttgttttgtttagtggaaagaaagttggttgaagaataatgtggtcagatttctagttctctctcttgaagttatagcttttttttttttttttttttggtgagacggagtctcgctctgtcaccaaggctggagtgcagtggtgcgatctcggctcactacaagctctgcctcccaagtagctgtgaccacaggcacccaccaccatgcccggctaatttgtttgtattttttttagtagagacggggtttcaccgtgttgccaggatggtctcgatctcctgacctcatgatcctcccgcctcggcctcccaaagtgctgggattataggtgtgagccaacgtgcccggcccctacattatagctttcttaagtctctattttagtcagggttctccagagaaacagaaccaataagatagatagatagatagatagatagatagatagatagatagatagatagatgatggatggatggatagatagatagatagatagatagatagatagatagacagacagactatatgggatttgttagagaaattggcttaaggagttatggaggttaagaagacccaggatggaccatctgcaagctggagaaccagggaagctggtagcgtggatcaacccaagtctgaaggtctcagaatctgggaaaccaatggtgtaattctcagcctgaagtcaaagtcctgagaactaagggggccactggtgcaagtctaggagtccgaaggccatagaacctggagttctgatacacaaggacaggagaagagggatgaccctcttctttttgttctctctgagtctcagccaattgaatggtgctcaccaacactaggtgagggtggatcttgcttccttattcaatccagcaagtccaatgctaatctcttccacaagtgacctcacagacacgcaaagaattaatactttgccggctatctgagtactccttaatccagtcaagtggacacctaaaatcaatcatcactgtcacctaccttttctgaggatcaatctcctgatatatagaataagagagaagtttacatattcactctgatcatagtatcttgttatttaagagccaaagtggatgtaagaaccagctgtccctacatcagactctgtatcagaagccatttgacaacacctctgacaggtgttcaaattctagtagaattcagagaaatatctgtctgccaaggcagctctatttattgccatcagttatcattaagtcctttctagtaatgagcaaaaatgtaatggtaaaatcagctagagcttagagatttgagtcctccttcttcacctggtcacatgtgagtcaactggcagtcctcattattcatctttccactaaattttaccatttatctttataatttttcagatattatcctgcattgttcagcaatctcatcttcaagtgtttgtttctctttccatccactcttccaaaaactctgtcccatcatcttggggaacattgaaagtcactgctggatatttgacatctggtgacagctttatcttctgtatgggtgaaatttggatttcaacattgatatcttttattcttgactcaccgttccatctactccacttgtacatgataatgtggcttcaatccctgctataaacatggcttaggatgctgcttctgcatattttgccccttcttgctccatagccacatatgaaaacttgtagcttggaccctagactccttttatccaaaaaaggagatgggttagatttccaaatatgcaggcatttccccacaggtcttctgcatacagaaaagataaagataaaaatggaaacttaaactaacctctggcaattcaatgggactctcttgtgtacatgatcacaagcatctgaaaaatgctagatgaggtattagatctgggagatactggttttatcaagcatatcttgtcatttccaaggttttctagttactgacaatgagtcactgctaatttttccatcctttttgtcacaattactgacctcaagaaggcaaagagtttcatgtgttctttgaatggaattgaagccttgtgcatccaaagattctgttgacttccaccctgcaaactgactttgatgtaccaatgactccagcaggctgggaagatattacttccccaggaaaactctgccccatttctgtttatgcaagtagctgtccttgctgaggtagtaggatcaactaaacaacaaaatcttctttgaggcctgagcactttcctttggggccaccttcttgagttcagcctgcatcactcactaaccttggttggtcacaggaacagttatgacagtacatctgcacagatgttgaaggcatgactagcttaagggccttctatgtcccttcctttaccactaaccttaggatagagtttatttctgatagttccagcctggtggtggtattctactcctttgcagaacacatttaagctactactctgatttcaattctcctctctacttcagcatctcagtgatgaactacattcttaaaacaatgttagtccacccagaggattctgtattgctagtcaatttttagaacaaggagatactcactggagtcaaacccaagggaagaacaattaggctatatatatatatgggtatgtgtgtatatatattgtgtatatatatatatgtatttgatagatttatgtttatttttacatacatgttaagtacaaatttggaagcataagatgacttctctgcaattattttgctatgtgttgctttccctttggaatctgtggatagaaactattatattaggttccacatcactagttatatttcaactaaaatttcataactggagctgggtatatatgcgtctctacttgatatagctgcatcatccaatgatgggtatatatgcatcatccagtgatgctgggtgctggaagatcgccatttagtacccaggtggcaattaattttgtggtattgtatttctgggtgttccctccattcctgacattctttatcatctatgattcaattatttgcctggctcaagaaagcacctttatttcccagtgaaccacgatgtgatagaggtgactttaagtaactggtttattgtgatcaacatgggtcaaaggaataggtgtgcagtgccactccagaaacttaccagagagtacttttgtcttctagaaagtgaacctggaggctcagtttctccagaattcatcttcctctgcccactccaccaacagaacagttggcatcctttagaccaggggtcccaatcccaggctgcagactaatatggtctatggcttaggctccatcattctagggtttgatttttagtttacttttcttgtctgccatactggaatttgctaatatagctgtggtccctgtaccttattatggtgtaacaaatgcaataaggataggggcttcttctgataattaagacttccatctgccctccgtaggttcaactttaatgcttcctacagatgggcaacagggctatctgctcagtgcaacccccagaattccaattgtctgtgctgtctccactccataccataactaagcctaggaagcgcctcctgaagttcttcactgggctgaattaagaacaggatcatcgttctctatctgggtgttttctaccccactgagagaggtgaaattgtcagaacaaaaatttaggaatttaccaaatgcagtttttttttggctgataattactattattaacaataatgacaatgataagcttagaaaaattaaacactaaacaatggctgccgtaagaacactgaaatgactttatagtttacaatcagtctttcgtctttcagggatatttttgtggacatctggtgccacattttagtatggggttcagagaatatcaatttacctgtttcttcatcatgctatattgagttatttactccacaataaaggtagacctactgatgagaaatgtcagggcaacctttgtgtatttcagttaccctcagcaaggtgctaccagctagcattagtgtatttctggtgttggtaacaggatcatgtgagccttgtagatgagaactccttgttgctcaggggatcatggaatgctgctctctttcagacagccatcttcggggaagagaagggtctctggcagagctctatgggtcctgcacttctctttcacctctctgccttgttctttcactcattaatttcttgacaatttattttccttaataactctagttccttctgtttatcttcttctgtcaatccttgcatccattgcaaatccatcacctgctctgaaataccacactgaagggaggtactcctaaagaccttccttcttttccactcttgttttacccttggcttacacaactgttgaacaactggagctgaggacaaaaagcacataaattccaaaggtcaacggaatcacttcttcatagtttacatctcctgtgtcttaagtagaactcaaatagaacataagcattgagaacagaggtctgagttggaggtcaagacatcgtttactagctgagggactttggctttatttttctcatctctaagagggaaaagataaaaatctacttcacagacctgttgctgggaataatgagataatttatgtacagtacttagcacattgcttggcacataaaaagtgctcaataaatgttatatatttataacattatttgtaacacctcgtctttcaaggaattatgggaattattctgtttgtcagactttgtttatggaaattatgtccttttgatggcccaatttttcttttccttttcttcatcttttgataacatactattaaatgtcaacaattcatactaattggaactagtgtaataagctgtcctcctcatcagtattagcagtctttgaacatggcataaaaatatgcccattttggcttacaggtccttgattctctaatgataatagtgcaggaaaaacattagatatggagttcaactttttaaaactgtcttgatgttggtgaccaataatttaaaagaggttagcatattttaaggtgactaaaactgtacaaatgaaattgccatttaaaaatgtctgttcatttgcttttctccaatgcagatattttaaataagaagaagtgaggcaagaaaaatgtaaacaaatattaggtataaactaggaacttagtcttttattttctgaatcagatggatgatatttaaaagtctgggtgaatgataaatggtagaaaaaaattaaataacagtcaatatgcacaacttaatggaggtaaaataagacttacttacttttatctctataaattaaaatgattttgaaaatcaagctgccaaaataaaggtgtggattttatcattaaagcaatgcccaagagacatgttattgagaaattatgtggcattggtttgttgttactagcgaaatgaatgcctaactgaagttgatttcataaagatttagtgtgtgtatgtacttgtgtgtctgtgtgtatatatgtatatatatatttgtatacacacgcacacatgtctaccatgtgacatgttgctatttttacaacttattttaaaacagctaaaattttctagatgagattaaacattttccataaaaataaaattacaatagttacctcaaagtttgtttatcctggaaaaaaaagtgctaacataagagttgttcaataatattttaccaaaattttgtcagaactccattaaaaacaattcaagaccacagtggtctaatttcaacactggcatcattaacctaacatatttatggaatattcactatatgttctgttttctgcttaagtatatgaattaaacatagtttaaatagtttactcaaagaacatgccatttgcttcattgatttcacactctctgacgtatgtattaataagaacataggaatccacacacagaaataatagcattcctatggtgcattaataaaaatgtatgaattttctatgtctaggacatgtattcattatttgggtgggtcttatatttcaggtactgttctgtgaaccacgtttatggaagtgatcaaaacaggcaaacatctctgccttcatggaatttacatttttcttggaggaggcagacaatagacaaaatatttagtatgtcagctaaatactaaagggaaaaaccagaggaggggatagtgtgtggtattgagggtcaaggccagtctcagtaagaaggcagctgtgagctgtgacaagaatgagggaagtgagccatacggagcccatgtaagagatttccaggcaaagagattaaaaggtggacgcctcctaaggaatggacatacctgccatgtttgagtaatgagaaagaagataaaatggctaaagtagagtgagtgagggcaagaataacatcagagaagtaaaggagtctagattgtgagcattgtgaacactggggaaggcatagactattcacaaggagatgggtagctactaaaaagttttgagaagaagagtatgtgatatgatttagggttgaaaaggattgctccagtttctgtgtcatataaccctatggtcttgaaactgggatattgtatccctgggggcatgaggtggcgtgcctagaggtacacaaaatcccaggatcagttagtcacatcttccctaaggataaaatttaccctcaagactatggggaaaaatgattactgtgttaagaaaaacatttaaattttatataaattattgtaaaattcccgtgattttaaaaataaagtatcaataatatttcatatttgtggcatcttaggctttaatcttacaggtttaagcttcttggccatcaggagtaatttgttgaaattgcaaattaatttggcaaaattatcataattatgtgccctgagagcttatgtatcttatgatagtgcaggaaacttgtgagatgaaatagttgagtgtatacattaaaataatttgatctaccaagtcactttaattatttatttgttcagtgttaaaaatatattttcataaccaaactaatacatcttaataatgttgtagaaatggaagtaaggtaaaatttaagaaactgggtttgttcttctgacttgcttattaatactggataagttactgaagagtgggctttggtttcctcgtctatttaatagtgtgggaacaaccttatgttaaagcaatctattcagtataactttagcacacagaaaataaaatattatgaatccaaagaaagtgtagcttgcaatcatgtattttattaaatgtatttatcagcattttggcttctgatggacatgtgtatatacatattttgtggataaaaaaattacttaactatttttatattagatactatcaattattctggctgatctggggacttttaatggtaaattttatatcaaatctgacaaagacagtatttcagacccgtttaagacctagaactcaccatgagttctaaaattggttctcagcaccatggacagcgttactgcaataggaaattaaagatcgatttggccccaaattaaaatggtgttgtaaaaaagtgggagaaaaaaaatgcctatccttttacttcaaattttaaaaaaatgatcctggccttcacaactgttcataagaagaataattaattaaacaaacatatattgagaacatcatatgctcagtaaaactttgattctataaatggtgtcatttaccaaatggattcttttgacaatttaattttctcttatctctctaagaagatgtaactacacactatagtatactactacaattatcaaatttcatgttgcatgtaacttgtcgtctgtatttttgtagttagattagattaactaaagatttttcaagtttgcctttaagtcatttaattttcctgccttatctttaacctttcaacattcctccaaacaatagcaacacaagtgttatgtgttaacttctctagtgacaaaaacttatacttctccacaaagagatgtgatgttcattatcaataagcttgacatctaaaattgttttataggagatacatattactttttcagatggtatataaagttaaataaatcttaagttttcaatgatgggaaaagcttccatttagtttaaacataatgtaaagaaatttgaatccccaaaatagaattataattctaaaaattcatactataattcttcttaaatgtttaaattacagttaattaaagtagttgatttcaaatagagtggaattatgggctgtacatcatttaattttatgtgctgactgctacatagccaaaggaacgtgaattaagatggttccacttttgaccaggagatggagctgtcatgtaagatgctgcctttatttatttatttttctaatttagcatgctgttttctaacagacattgggtaccatcgaatgactgtcagaacagaaagctaaggcaaaggagggaggatgctgtggtcatcctttcttgtttttttcttctttaatgaggatagagcacatgtgagattttactttctactccagtaaaaattctgaagaattgcattggagactgttatattcaacacatacgtggattctgtgttatgatttacatttttctttatttcaggtaagccagcatgattctatttttgacttatccacggattgttatctatgttaagaatgacatttaatataagatgtgtgctttgttagcttgtattcagatctaagagattcaaaagctctaattctagctgttgtgcaatttaaaatcttcctaggctgaaatgagctctgactatgacataccgtgttttattatttctttggctttctagctgttggtctctgtctctggctgtatttgtttaccttttaaaggtaaagctttcaaagtgaaggatgggttttcctttcacaagggaaatactgctccttaatgcagtgaacaaattgtagagatataatgaagcagattaacatggcagtggctctgccaaagctcagggctgagccttctgattgcagcctcagaggcaagccaatattctgagtccaaaggccttttgaacgatttctgctggctatgatggtacagattgtttatgtctttatttttacctcttagaaataatacgtaagtcatttctcccacatgaatcaagatttatgtaatgtgtttaatagacttggaatgagactatagaccctccctcctcccccagtatggatgtgaggagtatgctgaacacaaattaattgccatgaaaattgattttttctgtattttattggatttgggataatagaaccgtggccattttaaatagttgatattatagaaggtgctcacattttaaattattttaggtcatgtgacattatctgtgttaaagagtggaatgactcaggaggtggaagttgcatgcaacagccttgcttctctctgctgcttctcgtgatttcccttaaatccttgcttgattattattagaataacaggaataaagacatatagaaatcctatatttaaaaaccactgattttcatttagagaaatttaaagagattttacaaatattttaaaaatagttttgtaagattgtttttaaatgcctcacttctttctcctgtggaaagagctgtgtgtgtgtgtgtgtgtgtgtgtgtgtgtgtgtgatggtggaggtggcaggcggtggaggtgcatcaggatgtttttatctcagtcattgttttcctgtaaagaggtagttttgccatgtcagaggttgtgaaagctatggggaatgattttataaaatggtttggacaaaccatgaaaaaaacatgttttttaaatgtgtggaatctatagagtttgtagttctacatggctccatatacatggagatttttagaattacagatatttaaccatttatccccattatcatttaaagaaaagcaattgtaattgagagataaggtggatttcccagagtcacgtactaaattacaataaggcaaggctagaacagtgattgacagttccacatcttccatagcagtaacacaattcacctctagtgtgaacatatcaggatggcatagaccaggtatgatgatattcactgtgcgtgtgtgtgtgtgtgtgtgtgtgcatgtgtgtgtgtgtttggttatattctgtaaaatagagtttctttcagtatactgttgctcaatggcatattatatgcagtgctcaattttcttaggtttcttgtcaaagcatccaagaaaactgaatgacaactatggaaactctgtttcttagtatgcctttttcattattaggtggtgtatactcttctaccctatgctcatgaatgatgtggatggccacatggctttccctcctacctaatagaaaagattgtacatataggttaatctattgacaactataagaatatcatctcatgtccaagcctttcctcctttgttcacttactaggcacaggctttaccctacttcttcaatatcttgagaaaagaaagggattaaaatgtgtcatgtctacatatatttgtggaataccacgtaccaaccaagtgtccatggtagagataaagaaataattagtcaaggagtttacctcagggaggttaacatgcaaatatacatgtgcagataccactatatctcaaaataaggaatgaagaaagatgagaaagtgaatgggacaggaatatcagcacctatcttgtgccaggcaccatgttacatcctgggattcagaacaaaacaataatgttaatttctgtccatggggggctcagaggaaaagattttaggaggatgtgacagttaaattagatctttaagagtgagttggatataaataaagaaaaacagaaagttgcttattccaggtacataggtgaggcaagtaaatacaagaaaatggagaactataggcatcgtatagggaaactgtagagaaaggtaaataacccaatctgtttgaagcttttgttgtcggtgttgttgttgcagttgttattttttttcccctggggcagtaaaagtaaggataatgaatttggcttttggtgttatgcatttgacatactgacacatactcatacactggggtgttttataaggagctgaacatgttatatccatgtatcagattagtggttaaggctgaatgtatagatctgcatgcagttgtgggacaaaattaggtcatacataattaaacattaatggggaacaaaagagaagtgaagatgactgggggcataatcttaagattaggagagtaaaaaagtgtagaaaactattattaaattgtgtgaaaataatgtgagaagttgacggaataacaatatcataggaggccaagcaggtaaattgaatatcaaaatagggatcagaaacaatagaaaacccataaaggggttggttcataagaggatagagtaaagatgatggatttagttatcattattaggtcataaattaattttaagagaagaggtttagttgaatggcagagacaggggctctgaggactaaattggacatgagaaatctggggagtatatttaagttttaagaagtttgccagggaaaggtaggagagataacattttatacctggagaaagaaggtactgttgagggaaatttaactttagtgcagattagaaaaacctaagccctttgtagagtaaggtatgaagtcagtagagagaaaattgaggaaagaagaaacaaaagtacaccaaggtcgtggaagagacagaaggaaggtttgaaaatgcttttccttgacacaggaagaaagggtggttgatgataatgagcaaagctggtgtaactcactttgggtggcctgtttttcgaaattaaatccttgtttgtaagatgaagtatctcctctgattaaatcagtttgttcctcagtaaaggctattggtggcatatggactttctggaagaaataattaatcctcagtagcaaaacactttcctgcagaatatattttagtcacattaattcttgaattggagactaataacactgtttcacatgaagcttttacatacttcagttatcaatttcttattcataaccacctatttaaaaacaggttctatttaaaacttgtattgaattaggccaacacatatctattatacatatctagtttgattactaagggctggtttgccagtatctaaattaattattcaagttctttccttttttgaatccttcagtttcattagcacgttcaccaggacttgtgtagagagagtcaatgtcatggccgggtgcagtgacttatgcctttactcccagcactttgggaggccgaggtgggcggattgcctgaggtctggagtttgagaccagcctggacaacatgctgaaaccctgtctctactaaaaataaaaaattagctgggtgtggtggcaggcacctgtagtcccagctactcgggaggctgaggcaggagaatcgcttgaacctgggaggcggaggttgcagtgagctgagattgcgccactgcactccagtgtgggtgacaaagtgagactctgttaaaaaaaaaaaaaaaaagtcaatgtcaggaagctcctttcccttagggaactagcttccttatgaagcttcctaccagaagttccttttttctccatttcttctggcagttacagaatcctcttggggctttcttagagccaatctcctctaaggtgaatgcattttcttgcattcacctgtcatgaaatggcagtggaaagacttgaagaaggcacaggcgttggtgagtcatatatatattttgctgataataatgaataccattggcaatataatagtggccattgaaatattatttctattaggaaaagataacttgcatcaagaatatgagatgaaattctattaaaaaatgaaaagtagatgggccaccgtggctcttgcttataatccacttcgggaggtcgaggtaggcagatcacctgaggtcaggagttctagacaagcctggccaatatggtgaaaacctgtctctagtaaaaatacaaaaattggccaggtgtggtggctgacgcctgtaatcccagctactccagaggccggggcaggagaatcacttaaacccaggaggcggaggttgcagtggactgagatcgtgccactgcactccagcttgggcgatagagggaaactctgagtcaaaaataaataaataaataaatagaaaaggaaaggaaagaagaaaaataaaagctgacctctctataacactcatacgttagtgttatctcctctttatccatttgggtttagtgaaattggaggtcagtggaaggagaggtagaagtggattttgcaaatcagaagtataatttttttttctggtaatgttccattaaacttttcagtcaaaacctttatgtggaagaagacataattactaactacattaataataataatgaaaaaatcttatcatgtaatatcgttttgtttattagtgaaacattttagagaaatgatttaagaatcaaaggtatttaagtatattttatttattggaatgtgataaaatatttttgagggtaaaaaactataatttcagttaactgtttgtacttactaaaataacatttagggggataaaatgatcttaggaagaaattgcccacaaaaagtgttttgaattgtttagattatttttatagttttaagttgttaagtcttttgctggtatctaggattagttgggagtataatatgctttgaacatgaacatgtcttggtgttattagcaaataggtatacaattatgtgattgtgtaattactcagctacatcttgtgttgtgtgatatgtatggggaaatgacaacaaaataacaacagttctcttaagcatatatgaaggtagtgcttgaaatatcatgaaaaaaagaaagggagattatgataatgttatgataagagaagactgacactatccctcttctctgaataactgatatcaaaatgtgaaattctaaggaatgaagcaagctggtcagggcaatggtgatgctgtctctgtatcagcccagcatttccagctaggatattttccttcttttctgtgtgatacagtttggctctgtatcctcacccaaatctcaccttgaattgtagttcccataatctccacgtgttgtgggagggaccctgtgggagataatttaatcacgggggtggttaccctcctgctgttcttgtgatagtgagtgagttctcactcgatctgatggttttataggggtttttctccctttgcttgacacttctctgtcctgacaccttgagaagaaggatgtgtttgcttacccttccgccatgattgtaaatttcctgaggccttcccagccatgcagcactgtgagtcaattaaacctctttcctttataaattacccattcttgggtatttcttcacagcagcatgagaactgactaacacactatgtctcccctggattgagcaaatgagggagcccagcaattgtcaggcagctaggatctgtgaggagtgcttagacctacacttactatttcctgaattaacagtctagaagacaggagcctagttatacagacagggtgaccataaaatgtgtcatctaaaccaggattcagtttgttaataatttctccacaacgacaggtataaatcaagatggttccaggtaaactgggatgcacagtcactccacttctaagggtcaagaaacttctacatttaatcagtctattcaattaaattgttcatgattttgttgcctaagtgttagaaagttaaaatgtgaaatgacctatcaattagacaaagggcattcagctaagctgctccaattactggccttgataaatccatcaaccgaaatctctaatgattcttgttgaatccttagttatatcaccaggaatgatgtcactccaataatgttaagcctgacttcccagggaattcaactgtgtgcaaaaaaaaactgttagtaaattatgctgttattacaaacctagactcacagcatcataaatctattcaaatataaaagcaaaagcaggataaaatatggattcaaattatttccacatttgggtatgcatcttttaaggagttttcatgaatgtgtaatccttagaagataggaatttagctccaagaacactggaattttctccactggcactttatacccaaatcttggtaatgtcaatatgcatatgtatggcctcttaacagactatcctgtaaagtagttaaattattttcctccaatgatcatgttttttttacccttcttcagacatccactcccatggttttatcttgaaactcttcatggccagtaactgtggcctgcccataatccttccttcatacatcctacaccacctttctagcgcatttcatctacaactccaattcattcctttaatcccaccaggcctctaaatcactgatctcgtcatactttgattatctctcactaaatgtcctctttcctttcattagataaattccacagttattcataaaagtcactccttgtatatacatacatacctcacaaactcctccgctcagtgtttttttgtttggttggttttttggtaactgtctggctaaatcttcatcctcatattcagttcccaccttacttcctgcctgcacttttgcagctggacgcatgcaaaagtgggtccttcatgatgcccagcaatcatgccacatgtccctagattgatgcccagcaaccatgccacatgtccctggatcacttactctcccaggttcttgtacttagatttcataccctttcttttctcctcaaaccaccaatacctcctaccttgacctcactctcagttgacaactttgtttcctaaatcagtgagaaaattgaaacaatcaggagagatttttccccacaggtttctatcactgcatgtgccttcctaccaggttctgcacccacataacctgacctcccacccattaccttaaatcaactatccctgtttctatctaaaatcattcctgcatctgtgcactagatcctattctttcttacttacacaagggcatctctagagcattttatttctctgtctctcctgcattagtaattttagctctttattatatttgcatacaagcattttattattgctcttgtatttgaatttttttacttccttcaactatcttttttttattttttgttgtctttgcaataatacttaatgaaagtgtttgctgtattctctgtatctatgactctctttctattctcttttaaatttacgttagtcaagtttttactcttaccatttcaccaaatcagctcctgatagaaatctgatgaccaccctattgccaaattcatcagtccatcctcagctttcatcttacttgaccatccagcaatattggagacagttcattgctgtctcccccttgatacactgccttccgttggatttcaggatagcacacaattttgactcttctcttagcttattgttcactccttcacagtcactttactctttactctgccttcccagacttcattatttgggtgtccctgattctgtccctgattttcttttcttctttatctactttcttttgatcccattgagttttatggcgttaaatgccatataaatcatgatgacatccaaatatttacctccagttttctgccaaactgaacttgtgtgtctaactgcttatttggcttggatatctagaagacactgaaaagttaacatccctaaactaaatttcggatagttcttcccaaccaacctgctccaatcctcgctttccccaccttacttttttcaggagataactctagttttcctcatcctcaggctaacaactctagtatcatccttgattattctttttctcttatatctgacattcagtccatgagaaattcctgttgatttcaacttcagaatatacccaggatctatgtgctgccaccttcacactgctaccctgctcaagccaccatcaagtttctcatggatttctgccttaatttcctaattggactttattcttctacttctccttacaatctattctcaatacggcaagacagaagtaagattacgccacatccctgctcaaaattctgcaagtgatatccagttttctcagggtaaaaggcacctgacaatttccaaaaagtctctatttagcctggtctccatcccattacagtatttctctgaatttgtctccagtttttctttccctcattcactttcactatttcttgaatattgcaggaacacttcaatcctttgggctgtttcctctgcctagaaaccttttgcctcaggcacttgcatgactaacctcctcacctgtttaaaatctgagctcaaatgtcgtttctaaataatacctactcttaactatcccatttaaaaatctcagcccccttcctctaacttcattcttatcgtctcccacattcttctatactttcttttctttgttatcatttgctttgctttgttctttttttccttttctttcctttcttttatttccttcccttctaacacatcacattatagcatactgtatacattgcatttctcttgttggtaatttaagccccaaatgggcagagattatcatctgtttggtgcactgatgtttcacaaatgcttagaacacttcctgatctatagaagagactaaaaatgtttgtagaataaaataatgaatcttagaaacttaaatttattcctttaagcatactaaggggcaccatgctttctctttcactttattgggctgattcacatttacaaatgacccagtgcaatctgcctgatttttcattcagtatttacaccaatagaaataaaatgcatattagtgttataaaatcaaaccttaaatgacagtaaatgtatcattaatagttactgaagaccttttcctgatatatatgacatttaatgtttttctcttgtgacttcattgttctttgtttgcttgttcatcttctagattgtgagctcctcaaagtcaggaacttggtcatcatatctgtctcccaaataccacactcaatgtctggcacataagaagttttacaaatatgctgttttagtcataggatgagcatacacttaaaatttaatactggtcctgcatatgctgtgtcctgagctttcgctatgtaggttgctttaattcttacaaccgtgtggagcaaagcactggtctcattttacagatgtgataaagctagttattttgtccaggacttaatcattgcagtattctcactgtctaatctctagtgaccatatcacaggaagttaattatgctctttttttaaagataaattgaaataatgctaacaatgccagatttcttggaaatgttttgtttttagtttcagttagaagcacgccttagcttagtgaataagggacaatgaatgttatttattttcctgctcaattcagttgggaaaattgacctcatcgttttgtgttttcaatcccttaagtgtaataaaattatttactaccttagtttatataaatcaccaagatctaatggaagccacccagttaactctcctaaagagtcttcaagcatgttttgttctaaatctcagcttaagtattttccattgtttaccacctaatgtctatactacttgatttatagaaagtcttatctaatataaatatgctaactagctatgataaaattgatcactttccacataatcatatactgaaaaaaaattccactgaatatatttctgccctccatagattccaggatttcagtgaatttttcttaataatctctgcttcagaaatgtatataaacttttgtcctaatcaaatctaactcaaacgtcacttcctttataatgttctctctgttttcacatgcagcatatacttccattctactctgcacctatctctacacttgtaatgtgaagtagccagatatatgtggacctgggtcatctgtcatatttaattgtgtactatataaggacagatattgttttgtatggttctatcaagctacccagtatctacatatactgccctccataggaggtaatacatatgctgagctctactcatttaaaacttcaatcaaggtactattggaatttcttagttttacaacataactatgtcctacttcaagtgacctattaaaatagctctcaatagtttgctttaatacaactgcctttaatagcttttaaatttagtgtttgtttgagtagactttatggtgatttgtaaaatgatatggatatagtatatagaaaattttaacagaaatgtaaataataattataatttagggaaacggtgccctcttgctctatttcacacacacacacacacacacacacacacacaggcacacatatacacacaaaaaaaatctctatttccaaatcttgttcacaaggagctctctaactccatgagtaatttttacaaccaataacagattaaccaacaatagtcacacgaacatgtacacacacatatacacagcctaacttccccacatcattattatctatctatcgtcaagaatgcagcttataaaatacaatgacctggctgctataggaaaggataaaatgtaggttgcagtgatcttctgacattgagatggcaaggaatgtgttttttgcaggaatttcaaaacctgaattgtagaatgatttgagatactgccttaactaccaacaaatttgatcccatgttcttcatcatttttctgagattctggtttcccagatttctgaccttaattgactcaaccaaaaaaatctgcttattattaaagaccataaggagtaatcaacagaacaaaatatgatttaatataatattaaatatattttacaactcaaaacattctaaaagatgtgatggtttctgactataattcagtatatactattttagttttagtgacaatgtttctaacaaatacaggaaaacgtttcagaacctagtgtcctttacagcaagtagcaaagatggactcaggaatatgtaagcaaaatctagccaaaccaaaccaaccaaccagacaacaacaacaaagtgtgttgctgagtggctcatagacaaggacaagtatttgtgatgttctaatataataggtggtaagaaccaatggaacatctgtgattcaagtgagagtcgtggtggtagaaaacaaaacaaaagaaacgataatatgtgtagaaataataaaagaaaaaagtcaaagggtgtatttttacaagtacacgttcaaatatagcgtgatgatattttatctcaggaatttcatgcaaagtgctgcgtatgttgatatgttaggtctcttctggcccagtgacgattatcttgtgtggtttaagttctctatgccatgagtgaggtaagggcatttattccccagatccttccctgccagattactgtggttgaatgtaaacaccacaactcctgccaagtggcccctactccctctgattctaattattatttcctctttttgcctcctgctgctgctagccttggggtgcactctgctttgtttcttttccttaaccctaatatacctttgtaaataatccttttttgttttaatctctcaattaaaatctcctcttggggtgtgtcatctgttttctgctaagactttgattaatataatagctaacatttatgaaatatttactataatacaggcactgttggaataattcacatggatccaatttttttatcctcacaacgatcatgtgaattaggtactattatgactaaatttaaaatataaggctactgaagaacatagattttaaataccttcatgatcatatagctaaaaactaagacaaaataaaatccaggagtctgactccagagcagagcttagatgtgaaagtaattaatatgcttttaaagaaaatcttatattgttggtgtctgtataaatcccatagacaagatctaagtacgaaaaaaaaatagaagcaagtcaggaaaacttagttcacaggtaataaaactagacacagatatctaaatttagaaatatcccatgcatatattctgcatatctgaatgcatcaagatattaataagcatcttaatactttggaaaaagtatagaggggactttctgtcaacatttgaagaaaacatttaactagtataaatatatcatacaagttaactgaaaaatctatctgctgtctatcctcatgttcagttatcaagtatatatcatttaaatttaagatgaacaaaacactacttaaatcccaaagactcattaaagtaaaactagtaaatgcattttatttatcaattatttccctgaaaatgcacattctttttaaattaattttttatttaaaaaagagcatcatgccattaaacaaaaaaaggaaaattttatttactgggaaattcttgctgatttttacatgggattgtgacaaaaaaaaggtcagatttttatttttattcacagtaaggagagattttatcttcagggatgattacaaggtggggaaagggcaaggatctcaagagttaggcaaagatttctcttttagagagaggagtaaacaaggctagaatgaactggatggggagaagagggatttaagggtggtatgatctgacagagaatgttccactctgagaccagcctgctaccttgagggactatgtaaggggagggggggtgtggctgcttcatcttgaaagtaagtcaaaattcagggatttggggaaagtagaaaatcttaaccaaaatttggttaataggcattttgttcccattggtcagtggggacgggcagtttagcttatcatttatgaggtaaagaatgggaattttgaagttctgtgttgggtctcatcacaggtgaatgggggacatttgtaagttttgcctgtgctatatggggaagggtcattctttacagtaatttcctagaacatgaaaggataggggggttcttattatttcatctacataaactggaacagaagatgatttcactggtttattaaactcctcaggatcttctacctgcccttcttaacctttacattccaaattcacttctatctcctttctagagctggtaatctttcttccatgaagtttatgaaattcttcatgacatgcatagactttgacagttctgtttctgatgtgcataatacctaccagattgatttgtgataatatcattgagaggccacttatagcctatttatgactcagcaaaccatggtcttttacagcctcttttaagatggtttggagagataaagaataatacttcatgacatgtaaaaattatattaagtttgaagtttaatgtccataaataagttttattgggacacagccatgcttatttttatataaattttccatgactgttttcttcctacaaggtgagaactgtatacttgcaacagagacaatatggcccacaaagccaaaattatttactaaatcatcccttgtagaaactaagttgcagactctggtctatatagtcacattacactacaatgatcaagtatagatgtttgacagtatttaagtttctgaatgaatgaacctatatagagaaatgtataggtataactatattactgatatatcctattaattttattatttttctgttgagtttttagcttttctaggtacataattttgtctgaaaataattaggacaattatgtcatgcctttctttgctaatatttctaagcaatcattcctttacttatttctaactaatgtttacccccagttatattgtcagattttcctggaaaatattaatagtgcaaatttcctgctgtttcctaactttgacataaatatttatagtggctcttcactaaattacatttacatcaatatccttagtgatatattgatgtaaatttgatttgagacagatcaacttcataatgtaaggatatttctctttatgaagagaatttattataatttataaataaatgtgatatctgttagatgacttttaagcatctagatgtagttcatattctgttttggcccattaatgtcatgtatttcattaatatatttttaagtactgcaccattttttcattactattttggatcttattttggggcattatgttattaagtagttgtatgttttatttaagaagtttataactgtattcattaatgatattggctgtagatttcatttttgagggtgttctttcggaaaagtttactatcaaatttatatttgcttcaaaaatatttttgagtaattttcctatttttctatcctttgaatgtgtttacaaaatgtggaaataagttgtttctagataatatcaatatttatttgctagaaaataaaaactcacctgtaaacatgttttattgttattttaaagtggttttgcatatttcttttaactcatttccttattaatttcctgatcaagttttcacttcttacatcattttgacaatttcactttcatagacgtacacattttgtagaaaaattcaaagattttaacatagttaataattttgtttgcatacttttcatctctcaaaagctaagaaaaatgaattcatgtcttctacaattaacagctttgcataataacgtactactattaactttatgattttatgaccagtatatatttattagaacatttacaagtacaaaattaccttacttatccatttaagttatttgtgttgagatatgttttctctctaataacattggaaaacctaaatttttgtttgtatttgttgatatatttttgtccattttttatttttaattttggcaatctgttctatgcttctttcaaataaacatctgttcaacttttgctttgtgatctaatttggtactttgtgttggttaattataaattaaccaattcaaaatttattattttaaatatcagataatatttgatttttcttctcataacttgttttgtggtttctacttttcttatatttttcttgattttcccataggggtatatcaactgcagtttttcccatctttattttttcttcttctattttttctagctttttggaatgcatgtattgccttatttcattccattattttaaaagaaaccatcagcctcataaatgaaactatctattaactacccttacataagataaaaatattgttttttctttcccttccaactattttccagtttaaaaaaatgtacatgatttggtattcacgttaatttaaaaatgttctccataagcgtattattttataaccttactaaccacgattattaaaatatggctctatggtttaactccatatcctaacaattattaaaatatggctctatggtttaactccatatcctaacaattattaaaatatggctctatggtttaaccccatatcctaactaaggatgttaccattattaccatgtattaccatttcttctagcatcttctgtagagctgaagtgtatcttagagtcacctcttcatgaaggagatattgatgatatattttctcagctgctggtagatgaaaatatttctgtaatgccctcatgtaataatgataatttcagttgatacgcaaatatttttcttaaaaatatattttgttcttttatcctcagacatctaatatgcagaagataagatacagccagatttttcttccccgcccaccccccttggaaacaaaccattttcccctaccagaaacttgatggatgtttttctttatcctttgaccttctctagctcaacaggaaatgttttcatttggggctcttttcttttttttttttttttttttggtgatggagtctctcgctctgtcgcccaggctggagtgcagtggcgcagtctcggctcactacaacctccgcctcccgggttcaaacaattctcctgttttagcctcctgagtagctgggactacaggcacctgcccccacggccgactaattatgtatttttagtagagatgggatttcaccttgttggtcaggctgatcgcaaactcctgacctcaggtgatccacccacctcggcctcccaaagtgctgggattacaggggtgagccactgcacctggcctattttctttaatgtaaatactattccataatcctgtttgatttacagactctttttgttaaaagatttcatttgttcttgagattagaattcgttgcaacaactgtggtagccttagtttttccttttcaatttactatctttaatgatagcatcctcagggcttttgcaccatgtatatctttacattcttaataattatttccatctaattaactttttactctgagatattgggaagttatcacagacttccatttaacaaacttaatttctgcattttcaaatatgcttattactgtttatactgaacttatgaatttaactatttttacaggtatccacgctatatccaactggactcccttgctaactgtcagctttcctttcataattaaagtgtcctctttaatcttaatcagagtatatcctaaaattaaaatgttctcttctgcttcttattttgaacaatggtatcttttcatatgtttggcgattttttttcctgcttactcatcattacagaagatactctatgctgtataacatttgtattttagatgattctatcaaatattgcttatttcctgtaaacatctaaaagtcttttaggctgaggagaagctataactttgttttaacagttactagttcaacttgtaacaaagttaaaacttttttttttaactttgctcatttaaatatacagtggcctaaggaaactaagaaccaatcacagcagagtttgtgtaggtttgatggctaggaccaaccatgggaactgacgcatcatttcctgaatttagttgcgttcaagggtagtggtattcctgttctttgtctgacataactgtccacacctaaaagctgggcacagactttcccatctggtgggaaatatttcagaaacattagtgacacttgcagtgtcctgctattgctgcaaaacttgctcttccttcagcccttgccatggaattcaggtataattgtggtcttctattggcacatcctttatttacagacatacctcagagatattacgggttcagttccacaccaccagaaaagcaattatcacaaagtgaatcacatgatttttttgacttcctagtctataagagacttattttttcactttttaatcatagccattctgactggtgtgtgagatggtatctcattgtggtttttgtttctgtgttttgttgttgttgtttttgttgttgttgttcttgttgttgttgtttttgagacggagtcttgctctgtcacccaggctggagtgcagcggcgcgatcttggctcactgcaagctccgccttccgggttcacgccattctcctgcctcagcctcccgagtagctgggattacaggcgcccctcaccaggcccagctaatttctatatttttatagagatgggttttcaccatgctggccagggtggtctcgaactcctggcttcaagtgatccacccccctcggccttccaaagtgctgggataacaggcgtgagccaccacacccagcctcattgtggttttgatttgcattctctaatgatcagtgatgttgagttttttcttcatacgtttgtcccatgtatgtcttcttgacatgtatgtcacttgaaaagtgtctgctcacgtcctttgcccacattttaatggtggttttgttgttgttgttgttgttgttgtttttgttttttgttttttgcctgtaaatttgtttaaattactgatagattctggatagtagaactttgtcagatgcatagtttgcaaatattttctcccattctgtaggttgtcagtttactctgttgaatttcttttgctatgcagaaattcttaaatttaattacatcctatttgtcaatttttagttttgttgcaatcgctttggcatttttgtcatgaaacttttgccagatcctgtgtctggaatggtatttatgagcttaccttctagggtttttatagctttaggttttaagtttaagtatttaatccatcttgagtttattttttgtatggtataaggaagtggtccagtttcaatcttctgcatatggctagccagttatcccagcattgtttattaaacaggaagtcctttgtccgttgcttgctggagagattgcagagaaaagggattgcttatacactgttggtgggattgtaaattagttcagcaattatggaaaacagtgtggtgattcctcaaagaacttaaagaagaataaccattcaacccagcaaccccataattgggtatgtgcccaaagaaatataaatggttctaccataaagacacatgcacacctatgttcactgcagcactgttcacaatagcaaagacatggcatcaacctaaatactcatcaacagtggactggataaagaagatgtgatacatatataccatggaatactatgcagccataaaagattataagattatgtcctctgcagggacatggatggagctgggggctattatagtaggcaaactaactgaggaacataaaacccaataccacatgttctaacttacaagtgggagctaaacagtgagaactcatggacacaaagaggggaacaataggcactgggacctacttgagggaggtaggtgagaggaggaagaggatcacagaaaagtgcttattgggtactaagcttattgcctggatggcaaaataatctatatccctgttacatgcagtttacctatataacaaaccttcacatgtacctctgaacctaaaataaaatttaaaaaaagacctatgtttacactatactatagtttattaagtatgtaatagaattacatctgaaaaagcaatgtacataccttaatttaaaaatattttcttgcttacaaatgcaaataatcatctgagtcttcagcaagtcataatctttatgtgggtggaggatcttgtctggatgtcaatggctgctgactgatcaaggtggtggttgctaaaggttgagtggctgtggcaacttcttaaaataagactacaatgaagtttgttgcattgattgactcatattttcatgaaagatttccctttggcatgcaatcctgtttgatagcattttacccacagtggaactcctttcaaattggagtcaatcttctcaaaacctgccactgctttatcaacaagtttatgtaatattctaaatcctttgttatcatttcacaatattcatagcatcttcactgggagtagattccatctcaagaaaccacttactttgatcattcatgagaagcaatccctcattcattcaagttttatcataagattacagcaattgagtcacattttcaggcttcacttctattctagttctcttgctatttctaccgcatctgcagttacttcctccaccgaattcttgaacccctcaaaatcaaccatgacgactggaatcaacttcttccaaattcctgttaatgttgatattttgacctcctcctatgaatcatttatgttcttaatgacattcagaatggtgaattctttccaaaagttttccaacttactttgcccagacccattcacggaatcattatgtatggcacctgtagccctaaaaatggatttttttttttttttttttttttttgagacggagtcttgatctgtcactcaggctggagtgcagtggcatgatttcggctcagctcgctgcaggctccatcttctgggttcccgccattctcccacctcaacctcccgagtagctgggactacaggcgcccgccaccatgcccggctaattttttgtatttttagtagagacaaggtttcaccgtgttagccgggatggtctctatctcctgacctcatgatccgcctgcctcggcctcccaaagtgctaggattataggcgtgagccactgcgcccggccaaaatggatttcttaataagacttgcagatcaaaatgactccttgatccatggagtgcgggcatgaaaacgttaatctccttgcagatctccatcagagctcttgagtgactaggtgcattgtcaatgagcagtaatattttgaaaagaatagtttttttctgagcagtaaatcttaactgtgagcttaaaatattcagtaaaccatgctgtaaacagatgtgctgtcattcagactttattgtttcattcacagagcataggctgaatacatttagcataattcttaagggccctaggatttttggaatggtaaatgagcactggcttcaactgaaagtcaccagctgcatacagttatccctcagtatgcatggaggattcgttccaggactcagatgaataccaaactctgtgcagtccctgatataaaatggtgtggtggccaggcatggtggctcatgcctgtaatctcagcactttgggaggctgaggcgggtgcatcgcctgaggttaggagttcgagaccagtctggccgacatgaagaaacccccctctctactaaaaatacaaaaaaatgagttgggggtggtggcaggcacttgtaatcccagctactcgggagtctgaggcaggagaatcacttgaacccaggaggtggaggttgcagtgagctgagattgtgccactgcactccagcctgggtgacaagagtgaaactccgtctcaaaaaaaaaaaaaaaaaaaggtatgtgtggtatttgaatataacctgggcacatcctcccatatactttcaaacatctctacattacttatgacacctaatacaatgtaaatgctatgtaaacagttgtcatattgtaatgtttagggaatagtgacaagaaaaagttggctgtacatgttgggtacagatgcaatttttaaaaaatatttttgatccatggttggttaaactcacagatgcagaatccacaaatatagagagccaactatacttctaaattccataattctcaattttatcttttgtagaagaactggactagattatctctaaagtgtcttccacttctatatttcttccagtccacattaataggatcattgttaggctggtatctgtttctaaagtactacagagtaaatcaacaaggaaagcaaggttgtcttctcattcagaaggccagaggttgctaaatactccagttctgaattgtaagtgaaacagggaagttatatactaggtgattggtgtgctgttaaatgttttaaccttggctttgctctattaactttatctgtcctttttaaatcactttcagaaataaaataaggtgaatcacctcaagtggggtacaccaatgagttgagatcaatttgggacgtccttcatattgacctttgttgatacaaattatctttgtcacagaatattgctgtgattccattacctcctgttgtacttgcctgagcttcccattgttttagtcactctgcatcactgtgtacgatgtcaattgtgttttagatggttaaaaaaaaggaaaagaaacatttcagtcataccaaagtgttgatttgcaataaagagtataatttactgtggctctgggtattgattaatctatacaatgaaaatggcttttgagtgctttggttatcagttgaaattatctctattttttaagctgataattactgaaaaatgtcacaagtggaaaaaagggtgcaatacaaactagtgttttatccagatatctattctgcctgggaaaggtaaaaggatacacattatggttgcaatacggtctggatgtttcacttctgtgtatgtgtatttcttgcccatctcaaataataggtgggactttttttcacttttattttaagtgaaggggcacatgtgcagaatgtgcaggtttgttacacaggtaaacatgtgtgatgagggtttgttttacccattatttcttttttaaaaaaatttcaataggtttttggggaactggtggtgtttggttacatggatgagttctttagtggtgatttctgagattctggtgcaccctgacccaagcagtgtacactgtacccaatgtgtagtctcttatctctcaccccctcccaccatttcccctgagtcctcaaagtccattgtatcattcttatgcctttgcatcctcatagcttagctcccatttgtgagtgagaacttacgatgtttggttttccattcctgagttacttcacttagaataatggtctccaattccatcaaaattgctgagaaagccattatttcattcctttttaagacaaagcagtattcatatatatttgtatatgtataattatatatgtatatacacatagaataatatatatattaatcaacaagtgattaatgggcgtttgggctggtgccatatttttgcaattgtgaattgtgctgctataaacatgcgtgtgcaattatctttttcgtataatgacttcctttcctctgggtagatactcagtagtggaattgctggatcaaatggtaatatctacttttcgttcttaagaactctccacattgttttcagtaatggttgtactagtttatattcccaccaacagtgtaaaagtgttccctttttaccacatccatgccaatatctattattttttgattgtatcaattgttgcaggagtaaggtgatatctcattgtggttttgatttgcatttccctgataattagtgatgttgagcattttttcatatgtttgttgcccatttatatatcttcttttgagaattgtctattcacgtccttagcccacttttgatgagattgtttgtttttttctggctgatttgtttgagttcattgtagactctggatattagtcttttgtcagatgtatagattgtgaatattttatcccactctgtgggttgtctgtttactctgccaagtatttattttgctggtcagaagctttttagtttaataagtctcatctatttattgttgtttttgttgcacttgcttttgggttttgaagtctttgcccaagccaatgtctagaagggtttttctgatgttctagaagttttatggcttcaggtcttaatttaagttcttgatccctcttgaattgagttttgtataaggtgaaatatgaggatccagtttcattctcctacatgtggtttgccaattatcccagcaccatttattgaatagggagtcttttccctactttatttttttgtttgctttgtcaaagctgtaaacattgggctttatttctgaatcctgtattctgtcccattggtctatatgctgacttttatgccagtacctgctgttttggtgactatagccctatagtttgaattcaggtaatgtgatgcctccaaatttgttctttctgtttagtcttgctctgactatgcgggctctttttattttcatatgaattttaagattattttttctagttctatgaagaataatggtggtatttttatgggaatttgttgtgtttccatttgtttgtgttgtctatgatttctttcagcagtgtttgtagttttccttgtagaagtctttaatctccttcgttaagtatattcctaagtattctattttttttttttgcagctattgtaaaaggggttgagttcttgatttgattctcagcttggccactgttggtagtatagcagagctactgagttgggtacattaactttgtatcaaaactttgctgaattcgtttaccagttgcaggagctttttgaataagtgtttagtgtttcccaggtatacaattatgtcctcagcaaacagtgacagtttgacctcctctttaccaatttagatgtcctttattcctttctcttgtctgattcatctggctaaaacttccaatactatgttgaatagaagtggtgaaagtgggcaaaatcctgcactaaccaaatcccacagcatatcaaaaagataatccactatgatcaagtgggttttgtaccagggatgcagggatggttaaacatccacaagtcaataaatgtgatacactgcataaacagaattttaaaaaatcacatgatcatctaaatagatccagaagaaacatttgacaaaatccaacatcactttatgattataaccctcaacaaaatcagcatagaagggacataccttaaggtaataaaaagccatctatgacagacccacagacgacatcatagtgaatggagaaaagttgaaagcattccccctgagaactgttgtacagatgatttcatcacctaggtattaaacctagtatccattcgttatttttcctgaccctctcctcctcccaccttgtcacacaccctctgataggccccagtgtgtgttggtttcctttatatgttcatgtgttctcatcatttagctcctacttatgcctgcaaacatgtcatatttggttttctgttcctgtgttactttgctgaggataatggcctcgtgctccaaccatgtccttgaagaggacatgatctcattcttttttttgtggctgcataatattctgtgttgtatatgtaccacattttatttatccactctatcactgatgggcatttaggttgattccatgtctttgccattgtgagtagtgcagtaatgaacatatgcatgcatgtgtctttataatagcgtgatttatattcctttgggtatatacctggtaaagggattgctggatcaaatggcatttcagtctttagttgtttgaggaatcaccacaatgtcttccacaatgattgaactaagttacgctcccaccaacagtgtataagtaagtgttcctttttctccacaatctctgcagtatctgttatttttttaccttttaataatagctattctgactggtgtgagatggtatctctttgtggttttgatttgcatttctctaatgataagtgatgtagagtttttttatatgctttgttggccacaacatatatattgttggccacatgcatttcttcttttgagaagtgtctgttcatgtccattggccactttttaatggggttgtttgtttgtttttttttcttgtaaatttgtttaagttccttatagatgctggatattagacctttgtcagatgcatagttggcaagtattttctcctattctctaggttatgtgtttactctgttgatctatatatatatatatatatatatatatatatatatatatatatattgctgtgcagaagctctttagtttaattagatcataatcttttatcgtatcccttaactcaaaaaaatattgaacattaaaatattggtagcttttttcccactatcaacctccatagtaaaactatagttaggctttccttcaaatgaaaatccattcattttttatactgtttgcctgaaacaacacagtattctttcttaatagtattttcatgttgagctaaacaaacaacatttaaataaggaaaaaatattattatttcttttttattatactttttttccttcttctttctttctttcttcttttttttctcttaatatatggggtctaactctatcacccaggctggagtctagtggtgcaatcatagctcattgcagtcttgaaatcctgagctcaagtgatcctccctgctcagcctctgagtagctgggaccataggtatgtgccaccatgcctggctctttattacatttctgttataatattttctctattatatttcattttagttactaaagaatatgattttgaacttttttggaaagatcatcattttactctttgttgtagaagaaaatctatgatggtctaatgaactacggaaatattaacataattttaagaataatacatatatttgtattaccaaaggctatgcttattagtcattcaaacttcaactaattttttctttcatgaaatactcagaagtatctattcaccaccaccaacactccctccatcatttaatttcaagcagacttgggtttattcctggaaatgattttttactagtgttcttaattttagtcttcttctttctccaatatcttaaggcagaataatttttcttaaatgcaactttcattctttctccataaatttgaggtatcttcacatgaaattgagcttcaaagaagatgaagatgagtaagggccgggaaagccagggaaggtttctgtttcctgtgtatcaagtttcccaaacaagaatcttggagtcctcttcagcttctgcaactccttctcgatcaacatgcagtcaaacttcaaattgtattgacggaaactcagaactgcttagatcattttcttactctcattatttctgcgtggatttagacattcattgtcttttgcctgggctcttgcaataggcttttatcttatatcctaccgtaaaaggtttcagatgctacacatcctccatactaccaaagtaataggtaaaaaataaatctgattgtgtctgtacaaaaaaacctgttagaactaataaataaattcagtaaaattgcaggatacaaagtcaacatacaaaaatcagtagtgtttctaaacactaagaatcaactatttgcaagagaagtcaagacaacaatatcattttaaagcatcaaaaatattgaggaataaacttaatgaggtgaaatatctgtacaccaaaaactataaaacatggattaaagaaattaaataagacacacataaaaagaaagatatcccgtgttaacgagtggaaagaattaatgttcttaaaatacccagagtacttaaagtgatctgtggattcaatgaaatccgtataaaaattccaatgacataatagatattttttaaatcttaaaattcatatggaatcacaagttaccccaaacagtcaaagcaatcttaagtaaaaataatgaaacttggttgcatcacactgcctgatctcaaaatacattacaaagttgtagtgatcaaaacagcttggtactggcataaaaatcagcacacaaatggaacagaaccaaaagcccagaaataaatccatgcacttatggtcaataaatctttgacaaaagtaccaagaacacaaaatgggaaacgaacagtctcttcagtaaatgatgttggaaaaacttggtatccacatgcagaagaatgaaattggacccttatctcacactatatacaaaaatcaactcaaaatggattaacaacttacagaacacctaaaactgtaaaagtactagaagaaaatataggggaaaactctatgacattgatctgggcaatgattttttgaatctgaccccaaaatcacagaccaaaacaaaagaaataatcaacagaatgaaaagacaacctacagaatggaagaaaatatttgcaaaccatatgttcaataaggggttaatgtctaaaatatgtaagaaactccaacaactcaatagcaaataataataataataataacctgattacaaaatgggcaaaggacctaaatggacatttctcaaaagaaggcatacaaatggccaacaagtgtatgataaaattcccaatatcactaatcatcaggaaaacacaaattaaaactacagtgaaatagcacctcacattcattaaaaatggctactatcaaaagaacaaaagataagcgttggtaaggatgtggagaaaacaaaagtgttgtacactgttggtgggattgtaaattagccatcatagaaaacaatatgtaaattcctcaaaaaagtaaatacagaactattgtatgatccagtaattctacttctgggtatatacctagacattcaaagaagaattggtaccaaccctactgcaactatttcacaagatagagaaagaaggaatccaccctaaatcattctattaagtcagtatcacccaaataccaaaatcaggaaaggacacaagaaaaaagaaaactgcagactaatgttcctgatgaacatagatgcaaaaattctcaacaaaattctagctaagcaaatccaagggcatataaaaaagataatacatcatgatcaagtgggtttcatacccgggatgcagggatggtttaacatatgtaactcaataaatgggatacatcacataaacaattaaaaataaaaatcatatgatcgtcttaatagacacagaaaaaatgtttgataaaatacagtatccctttatggtaaaaaatcctcaacaaaattggcataggagaaacatacctcaaagtaataaaagccatctgtgacaaacccagaaccaatatcatactgaatggggaaaagttgaaagcattccccctgagaactggaacaagacaaggtgacaaaattagtgtgataaagagatatttacactgtcgtgtttattgcagcattatttccaatagacaggatacggaatcaacctaagtcttatctgtagatgaggaacggataaagaattgcacacacacacacacgcacacacacacacacaatgaaatactcttcagccttaacaaagaaggaaattctgtcacttgtgacaacatgaatgaacctggaggacattaggttgagtaaaataagccaagcacagaatgacaaatattgcatgatgtaacttaggtatggaatctaaaaatgtcaaactcacagaaacagaaagtagaatggtggttaccaggggctactgggtagggctgtaggggtggaggaatggaaaatgttggtcaaagggtacaaaagttcagttaaacaggaggaataagttctggatatctattgtactatgtaatgactgtagttaataatactgtgttgcatattcaaaaattgctatgtgtcgattttaaatgttctcaccaccaaaatataaatatctgaggtgaaggagatattaattagtttgatttaatcatttcacaatgcaaatattaagttgcacactgtaaatatatacaatttttaattgtcaattaaatataattgaaaaatagaacataataaaatattggattattaactctatcaggaaatgaagctttgagtaacattttactgtttatatttatggttttagccaccaagcaaacatggtggtcaagaacttggactcacattcaggttgccatagtccaaattcaggcttccctactcactaatgtgttcttaagcaagaaataaacttattttggcttcctttggtaaaatgggattaataattgttcctatcctacattttattgtgaagattgaattaatgaaatatacacgtaaaacacttaagacaattcctggccttagtaagatttcaacaaaaatggtcattattatgatgctttattctgttacatttatggattattagacagataaaattggacctcatatgtgaccattctattttcctttattcaaatggaaaaagaatgtcaattcaacatagggaaatactatatatgtataaattattacttaaatttatgtttttacttaaatatatgtttattaaaatcaagtgtttaaatttatatattaacttttacatttataacatcttatttcttctgaaaacaaccaacacaacccatttcttatcagtgataattcaaatggcctctttattctttcctgaatttttcaatcccaattcttctcttctgttctttttcagcagcatcttgcacagggctttatggaaaagactgaggtgctgtggtgaatttaatctcctctgttgtttactttaatctcttcatccagttactctgacttttctccatgcttaatgttatccctcttgatttcacagcttaacaatttcttctttgttcttgaccccatcctagccttcttatttcaaaagtcccctcaagcaagtttcttttcacattcttaatttcttattcttaataactccttttttatgccttcaagcaagtatggctctctctcatactgaaaatgtgctcttccgttctattttgctactgtttttattcacttaacagtaaatttcaaacaagttttgtgcacctaattctttcatttctttacaacccacccattgcttatccactcttacagctgtactagaattgtaatttaaatatgtatcatatcagtttttttgtaacattgataatttataatatttaacatattttgtaatagagttttcttcttaatattttactttccttttaaaaagcctcagcaagttgccattgactttcggcattgtatttgatacctctggactctggctactatcgccctttctgtataaaatctgtaacttgaccaaggcaagaacctccaccccaggccaactaattttctcatctgctattgtcccaacctgctttggtttctcctataggtcagagtcaggtcctgcttgggtttgggcctttgctcataattttcccttgcaattgaaatattctctcactccaggccagcctttaaagcctaacgtaagtaccaccacatccacgaagcctctgcacatcaacttcagatctcaatgctgttaaccttttataaacaaaaagcaatatagaataaatcctttttgggagcaggagtcaaaatgctagttgcagtcttgcatctgtcacttatttgcttggccagtgacgtagccttattaaaaactcaatttctttatccaaaagatgaggatgtttatacctccctgatctaccttctaagattgttgtacatgataagtaaaatactgtgcataaaaacatttgtaaaaattttaaaaatctgttcaagcttgtcattgttaccacagataaatcgggtactaccttgctgtcttggattgcttttgtatgttgttctcttatgttgttactggactcggtgttattacttaaatttctctgtatttattttgacaacttaattaggttttgaattctttggaagcggagagtatgaatggtattatgagcaaagacccagaaggagccacctcctaattattttggagagaattatctaagaggcctaattggacttgtgcttcccattaaagtatgattcatatcagagaagcttacagtcttattgctggccacatggagatagtacataggagatactcgatgggaattctgtcaactgaaagagagtcagcaagacagtgtaaacatggttagcgtgtctcagcttacccagcctggcttggtgtaaccaatatagactatggaaaataggaagtagacaaaaggtagtggagattgggctcatgccattttctttaattgaaatacaatttccataaaataaaatacagatatttttaagtgcatattactttgagttttgacaaatgtattcccctgtctggcaactgctccaattaagatgtagaatatttctatcaccccagtacccttttcctgtcaatccctccttctcccagagacaatcactgttccaatttctatcactatagcttagttttaccagttcaaaaacttcacataagtagaatcataaagcatgttactttttttcctggcttttgtcacttaacagtttttttttttagatttaaccattttgttgcatatgtaagtatatcataactttttgttgttgagtagcactctcctatataagtatgttataataatgtttcatttacctattgatgagcatttgagttgttcctaagttttaactagtataaaaaagtttactatgaacattcttgcataggtctttttgtgggtgtatattttcactactcttaagattgtcagactggataaagagcaaagttcagtggtatgctgttttcaacacatattttaaatataacacagacaggttgaaagtaaaaaaatcagtaaaagatacagcatgaaaacactaagcaaaagaaagctgatatggctatatgaatatcaaaaaagtagattttaagacaaaggatactaacacagataaaaatggtcattttattggggaaaaaaaggccaatttgtaacaaggcatgacaatcttaaaagggtataacaaagtagaatttcaaaatccaagaagaaaaaaatggcagaataaagtaaaaagcagagaaatccacaataattgttagagattttatcaattttatcccagtagttgctagaacaagaagacaaaaaatcaatgatatagacaacttgaataacattatcagggatcaaacctgcagacctaactgacatttatagaatattatacccaacaacttcagaatatacattcttttcaaatacacatggaacattccacaggtggtgttatgctaggccataaaattagtgacacttcatttcaaagagtatttgactacaatggaattaaattagaaatcaattacaattagatatatagaaaaggcccctttttacaaaattaagcaacagacttctaaataatcaatccatggatcaaagagggaaatcacacacacacacacacacacacacacacacacacacacacaaatacagtgcgctatacggagtgataattagaatacaacatgttaggaatctctgtgttcatgcctttaaccatttcatattatttcataataatcaagaacttattaatcaaattagcatttaattatcttcagatgaaatgagaaagagggtgggcaaaatgtagactgaaggctgagaattataggaattatggtcacaatcagccagtgttttaactctactgcaattgtaacaaagtcattggatcttggttcaaccaatgggaatagatcctttcaaaatgccaataaataaaatgctaaataaacattttggcaattttctcttgataaatctcttatagcaatgtgcataatgcttccattttcatccaatgaagtactagtgaaaatagtctagatagtgttaatattgatagaaattataactaaggaataagttagatggtgattaaagagttgaataagcactcaaagaaagttgtgaaatctctgttctgagatatgtgacaaaaccatctgtctctgattttagtttaatccttaatgaatataagaagcctgactaaatgatctcttgggaggcccccttccaaacacaaaacccattattttaatttgcattggttatagtgccaacacaaagaaaaaacacactcaagatcttttttataaagtagggatttaattctaaatcttagtcatatcttacaatttataaattctttaatgttgaaaaatattttctttgtagaactaaaaccattctctctctctccctccctccctctctctctttctctctgtctgtttgagaacccaaaataaaatgtagtttataatttgtgattatgatttgaacttttcaaaaaatgtatgagtcatttcttgccctttgataatttccaaatgatgatgctttgcttttgcttttagagatcagtctttgcttttaattatttccatatattttggttgggagtgagaaaaataaacggaaaatgtcactcatgagtgtcagcctggtaatgaatatatagaatgcaaatggtcactttactgggggagacggtaagtttttctgagccttttgcccttttatcttctccagcctgctgttttttctgttttcatttgtagattaaactggtgcagcaatatggtggggtagaaatcacatgaatctgacatatgtgggctcctgtatctgtgcacatggatagagatgctggtcaaaatccaaccatcttatgagttccagacatacttttaaaattcacatgcatgaagctatacaaaatattttaaaatacagaatccaaaataaaatttctttttgtataaaatactccttctcaattcattcctgtaggcaaaacttgatatttaataatattaagaaaaagaagaaaatgaattcgaaatattttattgctttacaacacaaaaaaaatggtttacttactcccaataatctgaatttattccaatagtatatttatggatttaaccaggagagggattatccttattctaagtaggggagaaaaaattaaaaaaaaaaaaaaagaaaggaaggaaaaactccagtggtcaagagattgagtgaaccgaggaacagagtggttatttgttctctgtaatgtattaggaatgactggaagtaatggaatatgctagtgaatatagctacccctatattgaatagaaaaatacattaccaaaacacttttcacacacgaaaacaaaagaacattttgtacaaatacctgatgtgtagctatatcatttagttgctactattgcctagtttaaaaattgtaccctggttaaaaacagaatcccttttctaatcttttaagatttcaacaaatatttccaggcattaaggacaggcagtattttgccttgtccacttctctgacgtctagctgtttctagttgctaaatttggctgctgtacttttcatatccgtaaagctgttcttgggtccaccctgcaggcagccatccaggtttggatttttccgggcagcccgggcttggctatcctgctttcatcatcctccttctgttctcaagccaaggacatagaatggcagagagctcccctttatttctggtatgattgttgcttctccgggtctggcttgggctctgatcttgacatttatgtcacaggtgtcactggtgaaactgctgtgaaaccagatggcaggcctaactgaggttggcagcaactgctgttgtccccagtgtcactgctacctgctctactttcttattcctgttgatgttctactgccttgttttattgcattcgttcaatgttaccttccaacaggccttttcaaatgtgaacaccattccataactcttctgatgcattaattccccatggaacccattcatctagggaggctcatgacttggctagattctccactttccttcagggcttttgttaaacctcatttcttcagcaagcctctccatgaccaccctatctaaaattgcaatacatccttctcctccccaaccctgattattttttctctctttttcataaattttctttccctggagcaatttgtaccacatgtcagaatctatatttgccttacttgggcattatctgtatccctccactgggttacctagaatataagctccacaagggcaaggttttgtctgtttggttcattactgtatcccaagcatctggaagaagactggcccttagtagacatttcaaaatttttgttaaatgattagctggattgtaaatactgaaagattaggtttattgtttttgatttggtttcattcattgtgcttagtacagtgctatatgtaagagcttgaaagacagagaaaagagcattagtagtatctgttatgtatactggatgttttacttgagttatacccttttttttttccaagacagagtcttgctctgttgcccaggctggagtgcagtggtgtgatctcggctcactgcaacctccgcctcctgggttgaagcaattctcctgcgtcagcctcccaagtagctgggattacaggcacccgccaccacgcctggctaatttttatatttttagtagagacaggttttcaccatgttggccaggctggtcttgaactcctaacctcataatctgccctcctcggcctcccaaagtgctgggatggcaggcatgagccactgcactcagccaagttatactctttaatcctcacaataatctaggtattttgaccaaagaaccacagctagaaagtaggagagtaagatgtaaaccagttttgtctggctctaaaattactgcctttttttgtttgtttgttttgctcagaagaattggtcatgccaaatagtaataagagttcttcaaaatgaatgatatttcataataaggtgtagctagaaacagtttgccaaaaatacttaggaaaaaaaaaagatttgtgaattagctccctaaaaatacaaacgcaacaattccaaaagaaggaattccaaagttattgggccatagtacttttgtgttaagtgcctggccctccaaggtaactaattttaatttaaaaaaaatgtctggacacatattttgacatgttttgctgaaaatatatttataatatgagcccaacaaagacaaatagattcattcttggtgctaaaaaatgacaagaaaacttctacgccattgctctcgaatgctttcttcattgtagtccaagaaaatttgattagttgtattttaaaagtaagccagatttcactggtaaggtctttccttacttcatcttctcccttgacaccttttgatttggttgtacaaatgcaggttaggccccattattaattcagtaccacatatttatgaagaccccacatgatgcaatcagcactagagactggaagtttttcatcaagcccagtaagcccacagtaaaaaggaatcagtctcaatcactcatcaggaagctaacatgttctgtgaccaccttctggatgttgagttatctctaatccagagtttatgtcaatgatcttaaaaattagttcaattctggatgggcgcggtggctcatgcctataatcccagtactttggaaggctgaggtgggcggatcacgaggtcaggagatggagaccatcctggctaacatggtgaaaccccatctctactaaaaatacaaaaaattagacaggtgtcgacagagcaagactccgtctcaaaaaaaaaattagtttaattttgtcttcattttttcttacttagtaaaagataatgtaatgcctggtgtttttttaaggcaatcatagaatctggaggttgaaaatatttggggaattatacattctaattattcagacaggcatccttactgcagtatctgctagtaattgaggattatttcatatttaaaaatactcatatttttatgcaaatctcagaatttgaacattgttacttataattgttaaaaagtaaattgagaaacaataaaatgttaaagagcttatttgagcaaatggtaattcttgaatcagctccaaaccaaaagtggttctgaggccacttgagagaactcaaagagaaagcttttatagggagaatgtagaaataaagcaaaggaaatatttaattggttgcaattatatagttgccttatttggtctatcttgctgaaaagttcctacttgtattactacatgagttggcagcttctgattggttagccttaagtttcatttctctttaggcatttataagaaatagttcaagttgagttttgcatatgtttgcaaatcaagcaaagtttaggttacttctgagggccacttggatttgtctgctcagaacttcttcaagcctggtctgttttaatttacttcaacatattttatcaacattttgctttctataacattcatagtaaccttagatctgccctctagaatcgacagaggctatttcacccccagcctcttttcaacatgagagtttgctcaatgctgcttctatttgaaatcttctgttattcagctcttttaatgaatatctactttctgcaaggcattgctttatgtactgcaagagttacaaagaggagtacatcataatcttacttgaaggcaattacttaaatagaaaaaatggtgtgtgaaaaacaaatgttcttaagcttgaaaaagtgaaagtgtgtacccagctggaaggatgtgaaagtgtgcatggaaaatgtatttgagatttaaactgctgttgtcttctgggttgaattggtgtcatgaacttcaggtagtgaaaatagcttaaacaatacccctgaagtggaaacacagagattagtgtgaaaatttagaagaccgtgagcacattatattttgtgtgtaccataagacactggtagagtatattagtccattttcttgctgccataaagaactgcctgagactggaaaatttacaaaggaaagaggtttaattgattctaagtttggcatgactggggaggcgtcaggaaacttactatcatggcaaaaggcaaagaggaggcaaggcacctttttctccaggcggcaggaagaagtgctgagcaaagaggggagagccccttataaaatcatcagattttgtaagaactcactcattatcacaagaacagcactggggaaaccacccccatggttcaattacctccacctagtctctcccttgacactcggggattatggagattaccatttaagatgagatttgggtggggacacaaagcctaatcctatcgtgggggttggtaagagggaagactagaaaggaaagctggactaggcaaagtggctcatggccataatcccagcagtttgtgaggccaaggcagggggattacttgagccaggagaccagcgctggcaacatagtaaaaccccatctctacaaaatgttagaaaattagacaagtatggtggtgtgcactatagtcccagctacttaggaggctgagatgggaggatctcttgggcctgggaggtcaaggttgcagtgagtctttatcacaacactgcactccagcataggcaacagagcagaaccttgtgaaaaagagagaacgagaaaagaaaaagaaagaagtcaagaaagaaaggcaggcaggcaggaaggaaggaaggaatgaaggaaggaagcagggaaggaagcattctttacatgccaggaagaggaatcttagtgatgagaaaactgagtctctgagagtttgagcctcactcaaggtcactgagttgctaaatggcagaactggatttcaatcccaataatttgattcaaaacccatactctttcccatataccacattatctaggaggtgtcccaagagttctgaagggggagttagcttccatctcaggagacgaagagagtcttcttagaccaaatggtatttgagataattttgataagtttatgttgaattcattggttgagatagggaagacaattcatttcaaatggaagaaactgagcaaaggtacaaaggggaggaagtggagagattgggaacagaaaataattaatctttggttgaacatcatcaaaaaggtataaaggatggaaatgaaagctaagatcagagcacaaaaggataacatatttgaacttttacttggacttaaatgataaagtttttagacagattattattataactacagccaagtttttaaaaatattatattagtaattatatataaaatagatggatgggaaacaaacggaagaatggagattatctgtttagagtaacccagttaattataataaaatattgaatctgcatgttggcaatggaatgagaagggaagagatagatatgtaatttcagaagcacaattcacagaacttgatagagattagtggaaggaggagggagatagatgacattaattctgtgatatggtttgcctgtgtcccacccaaatctcattttgaattcccacgtgttgtgagagggacccagtgggaggtaactgaatcatgtgggcaggtgtctcccgtgctgttcttgtgatagtgaataagtctcataagatccgatggttgtataagggggagtttccgtgcacgagccctttttctttgcctgccaccattcacgaaatatgtgacttgctccttcttgccttctgccatgattgtgaggcctccccaaccaacaggtggaactgtaagtccactaaacctctttcttttttaaattgcccagtctcaggtatgtctttatcagcagcgtgaaaacagactaatacattctgtaaatttgaatgcaggagatagaacctttaaaagcaatggtaatttgtcttgcaaggcctatgtttatctctactatgtactcagactattgtacagcctggaccatagaaggtatgcaatacgattatttatctatgtactcaatccctactctgtgccagggattgtcctaagcactagcgatagagtaaaaagaaaagaaaaaactcttgttctcattagtaaaataaacatcacgttgcttcacccaaactagatcaccttagtcccttttattgttatctctgtgtcactctcctggcaggtgagtgtttctgcttttaagaatctgtacctgtgtcttgctttagataactgtcttcagattatctgagtcattttttccacttcagcaccaagtctgaagtgccggtgagtttacgtgcccccacccatatccttagcgaagactgactattgcagtcttgtgaaagtacagatcccttgcttcatatagggacaaactcaaatacataatttgtactccaaagttgtcctgtgggatcataataaagccacttctgtagggctttgtttggaattacaccttagcttggctccttccccatccagacctgcagctttacccactcctttcctgattgatcctaggagtacctccttaactaatcactttcccattaagccacatctcagtgtctgtctctggggaacctgacctaagacatgtagtttacattgcaatgtacagagacaaataataaacaatataaataattattaataagtttcatactaggccgggcatgttggctcatgcctttaatcccagccagttgggaggccaaggcaggagaattgcttaaagccaggagttcaagaccagcctggccaacatagcaagaccctcatctctacaaataaaataaattagccaggcaaggtggcatgtgcctctagttctggctacttggaatgctgaggtgggaggatcactggagcccaggaggtcgaggctgcagtgagctttgccactacacactagtctgggcaacagagcaagaccctatctctaaaaaaatagatattttattaggggaagttgaagttatagagggaaataaggaaggagatgtggttagagtgtgtgaaagtgtgtgtgtgtgttttcagttgtaggggatggtgttcaattttatatagcatggttggggaaggcatcactgggaaggtcctatttgagcaaagactagtaggtgctaagggaatgagccatgcaaatttctgggaggaaagcatttcaagcagagatgaaagcaagtgcaaagccatgagctgggagtgtctggagtgcttggtgagaagtagccatgtgagaaagtacagggcaggagacgacatgagagaaggaagtgaggggggtgggcagaccttgcaggccaacacaagccatggctcatactttgacttttcctctgagtaaatagagagtcactgtagagttctgagcagaatagtgactcgactgtttttgcgtttcaaaggaatctggctaccacattgagaataaactgaaggaggccaaaaattaagcatggagaccaatttagacattattaccagggctggcttcgtgggcagccacacaaggctccatgctaagaagggccccacacatggagcctaatgctctgcagtcaccatcttgataattttaataatttgtgtttgaatttaggttttgtaagtgaaagtcatgggactgtggagcatgttctgggtgctcagtgtctggtacttcctgctgtggataggttcaacactacctgcacccctgctccctgcccaggccactcagtgccccctttttcacctttttccagcaactattgccaccctccaacagggagaatgctgggctgctggtgggaagatgtctgtgttctgctgcccttaagcctgtcggggatttgatcacaagctcaggtcaagttgcagttaggaatgcacctcaggctggaagtgccaaggcaggtgagagactcagtgggagagagcttctcacccacctctaatccaggcatgtcaggtgcagagttccaatgctttggggggttgaccttccaccagtttgggtggcaggctcatgggaagggtagattgacttctctgaccccaattgagggcaggttgcttcagtccagcaactggtgggaaggggaatttggcagtgggggatgggggcagacattatcttctgctctgaatcagggacactcatatggctctgtgtttgccctgcaagtgtttccctgctccaatgagtacaacattaaatagcaaataaaagccatcagatcagtagacagtttgcataaataagaaaaaagctatgttgcataatcttttaatggcccctttttcctgctacttaaacaagtgtcttcctcttttttttagagacaggtcttgctatgttgctcaggccagtctccaactcctagcctcaaggaatcttccagtttgagcctctggagtagctaggattataggtgcaaggcactactcgcagctggggcctcaacattatcagttttgcactgtacttcataaattactggccctagcaattgcagtaatctaagtgaatgatggtggcaacttgaaccagtgtgctaatggtagaggaggtgagaaatgattggatccaagatatattttgaatatagaacaaaaaggtcactagaggttatgacatagtgtatgagagagagaggaaggaatcaaggatggctgtaaggtttttcacctgaggaacttgacattatttgttgaatacataaatgatgtgtcctgttatgtatctgttcatgaaacacattgatggtaatgaagatgcagtggcagaagcaaatcatagaagtatttcctagcggtagaaatattaagacttggtgacaaagatcataaattaaaggaaagggagtgaaacaactaagtcaggaaaaatacttctaacttaggaaaaaagaagaaaaaggaaggaaatggaaatatttaacaaatggtataaatttctaaggaagataagttcaattttggaaatgttcacctgaggctccaagaacctacagcccaggagaccttagctataggttgttgggaatatgtgataggatcctgaatgagcactcaggactagggatctatgttgagggcatcatataagtgtaagctggagtcacaagaatggttaactcatcaataaatgaatatgaggagagatgtatacagtattgcagaagagagttccttgtaaatgtgtaagctcatcctccatgcctgggataagagtacattcattatggtgcatgtgaaggtattgagaaaacaacttccgctttctggcagaggttaagctagtcccataatagcaatagcaagattataaggggaaactatgttatttgtactgggtcacaaatttgtctaatagcagtggacctgtgggattctataaacatcttaggtatagaaagggatccagtgacttttagaaaatcagacttatatcatatttcccttcttaacttgtagctcagtggtgcatttcttaccctgggggagaaatggaacatggactatatttagtatttgaaatcagacacatgtgaatttaaactctggctcagagtggcaatggcaaattattcaacatccatacaccctgatcactcccacatttgaaaagtgacaacaatactacttccttcacaaataatgcaaggatcaatgagataacaaatcaagaagccagtagtgtgccacaatttaggtagtgttgacattacatgaagaaatgaacccatggtctcctatgctctccttcactcctctctagttctctctctctctctttctctctctctctctgtctgtttttctctctctctcatacacacaggcatgcatacatacactttggaatgtagctattttttgagcattgttttattattatttaatttatttatttatttattttttgagacagagtctggctctgtcgcccaggttggagtgcagtggcgcgatctcggctcactgcaagctccgcctcctgggttcatgccattctcctgcctcagcctccctagtagctgggactacaggtgcccgccaccacacctggctaattgtttgtatttttagtagggatggggtttcaccatgttagcaggatggtctcaatctcctgacctcgtgatctgcccacctcggcctcccaaagtgctgggtttacaggcgtgagccactgcgcccagcctgttattattattatttcaaagaccttctgatcctggctctacctgctcattcatggggaagatgcccagacattcagatctactgttccactgaggagtttagaggctgctttttctcccccataaaagagaatggggactgatggaaaaattttaggtagaagtctttgatgaaaagaagaaatgaggaaaactgagctggaaatattcaggtatatcaaacaagtggaaatacctcaccctcataaagagaaccatatcctgggaaattttctttcttacctgattattaaaatccagaattggaattctgtgattccagtataatccatccatcaaaaccaaactactatgtaatcagattagaggtggccttcctgctgatgagccatttagagacggttgaatgagttcttggaagtctgctaacatgcacttaatacatctgaagattttaaaacgttgcagcaatgtattgccagtctacctacagtaaaataaaaatgcttccattaaaatacaggtatatttcttttatatagttaacttatttttcctaggaaagttagatgtaaagtgattatctatacactgaatcatatttcccagagatcatcatagtaagattggtatgcatttctgttaaagaaaagcaaaatgtgtcttaggacataacaatcatttaatggaatatctaaagaatgttggtcaagcattttgaacaagatgaagatggaaccagggctgggtgtgggcagtagactgttccaccaggagccaaagatgacagtgaatatacacttgacatcagtcaccaatgacaacctaattctacgtggcatactggcctggatcaatgagtctcctgaagttgaatttgacaaagatggaacaattgtgctcaggggctgcttattgttgctttatggtcatcctcttccctggctccattgccttgaagaaggtgaaattccatgaatacattcataacttcaaaatactacaagcagtatttttaagagggtgggtgatgacaaagtaatttctgtggacaattaggaaaaggaaactttcaggacaattttgaattcattcagtactttaagaagttttttgatgcaaactgcaatgaaaaagactataaccctgtggctgccggacaaggtcaagaaactgaagtggctccctctattgttgctccagttctgaataaaccgaagtaacctctcagctctagcaatccagctccacataggaccatttccacaaataactgcagcttctaaggctgcagcttctaaggccccagggtggtattaaaaaatcctggtgtgggcagtaggagtgctgcagggtcaaattgatgctagcaggtcagtgtattgaaacttggcattgaagccttggagaaagagatttctacttcagaaagctaaggaacaatgaattgatttgccagcagaacgaggggcaaaatgaccctgtattgcagaggactgtagacatgctctgtgccacagatgaagacttcgtgatatccgatgaaaggggcccacaggaggaacaagaagagttttaacaacccgaaccaggagagccttatccgaattctgcactccaaatcttgtgcttaactgttaaatactcccttttattattcttagaagactcactggtttctttttatgagcatgaagtacctctttttaaagtgcattttgcagagtttcactccttttttggtgagtttgagttgggagttttactgtgcagcagagcaacattaacatctggttagttcacctgggaaacaaagaggctgaacatggggctcaccatgtgcatgtaggtaacattgattgctggagaaggtgtttggtaatatgccgaagtggagaccttggtacagaaatgtgaagactgaattgaattttaacctaatgtgaaatcttggtagagaattttgtaataagttaatgcctaaagaatatttaaaatatgcttccatattcaaaatatcaaatgtaacacatcagaacatcttatgtgtttgacattgtatgttggaaggaagggccagacatcagaacctttggaacctggtgtcatcacaggccttacagggctgcttgcaacctcacaggcctagactttggccccaaaggaaagttctaaatgttgctctgtaaatccatttggtgtcattgaccaactgcatccaggctaaaaagcaagaggtattgttgcctggatgaacacagggtgtgtttcagccctgagatttttgggttgaagagctctattttcattgaggatttctctggaaatttttccaattatctctgaaatttctatgtattacgcttttttgggaaatgaggtgtgtccaattctttcatctaacaacgcttttggggatttgctcacatctctgagatttgaatggaggttgtttcccattttaccatcctttagttttacatttaacatgtcaccagtggtgaatccatagaggtctgcagcaaacttgatccttgcctcgttggaggaaagaatttggctgaggggaagaagtaggtttaaggtagagggagagaccgaggcaagttttagagtaggagtaagagtttattaaaaagttttagagcaggaacaaaaggaagtaaagtatacttgggaaaaggccaagcaggcaacttgagaggtccaagtgccctgtttggcccttgacttgaagttttatacattggcctagttatggcctggttacggcgtttgcatctctcttccctttattttcttttggagcaggctgtacacatgtgcagtgatctgccagcacttgggaggggcaatatgtgcagtgtgtttactgaagttgtgtgcatgctcacttgaggcaattttcctttaccagtagagtgttcctgagaaggtcatatgccagttaaactctgccattttgcctcttaatgtgcatgcttgagctcactcacccaattcctgagaacttatcaggaagcctctgatcaccaggttcaggtgttttttttttttaagccattaggggactgtctttacctggtgctggctaagaccaattattattttagacagtttagtaaccacctgaccgtcatctgatggttgcctgacattcctgagggaggggctctctactgccttgctcatgtctgcctaactacctactctaacattgcttctcttctctgctccccttgcccactggggacacctctttgggttcttaaagtttgcagcttggagttggaagtgcagcaggcaggtgggcacactgcaaattctttgtggacctctggcaaagggaataatcagggaaggctctggttacctctgcaaagctgggatgtttttggtatctgctgtccacagctctccagttctctgaatactttatcagtacactaatctcttaagagataaaatttattagtgtgttgctaaatgttcattttcttttacagaaaatacagtaccatgtctgaattattcatatttaaaatttgttattccttaactctccctcatttgatttgtgcacaacctattccatccttttgtttggcagaagtttgcaaaatatgtgtcactcactgagattgttcagcccctgatgcatttgtattgatttgtgtctggtgatagcttgtcctaaaatgtgtgtaaaaagtaaacattttataacaaaattgttgtttaatggatgctttgtgtggaattcagagaaaaatccagattcagtgattaacaatgccaaaaaatgcaagtaaatagccattgttcaaatgacagtggtgctgtttctcttttgtggttttttagacttttgttacctaaaattctgctttattagcaactcatttttcacctgatgtttcttgacaggctttttttttctattttaagtagtttctaaataatttttttttatttcaagaaagagaaaaaagaatattgctcaatgcacctaatataaaaataaactataaaaaataagttagtttcatattggaactaaacctagcattaaaaaaaaaaaacagcttttctcaccagtaaacactgaacgtgagttcttcatattctactattctctgtttctactctacagcagaaaaacacgtaagtctggttgttcagaactgtctccccattatggaaattgttagttacagaaatgggctagtggattcaggttaaaaagagaaggatacatatacatgtgtgtgtgtgtgtgtgtgtgtacatatgtgtgtgtatatgcatatatacaaatatatatcacagcaagtaacaatcaaaaagttgataagaaaatagacttgaacctatggaataagaaagctagaatatctgaaaaataagtattcaaataatatttagcaaatcttttattggtttatgtttacattactgtaatgtaccaaagaacccaaaaaactacaattgtagaacagaacttaaaaggccacttccagaaaaaaagactttccttagtgttttcagctttcttaattcttgggcacaaaattttctaagtctttgcgcacttatccacaaccactaattacaatataaactaatataaaattatgtcctcatctatgaaatttaatatgccattttcttagtataaatttatttctagagcatgtattattaatgtggatctttgtagaaaaaaattgtggtacaatgtttattactacatacccttatttttttaaattgtcttatctttcatataactttttgctgatgtcatcatgtgtaataacatgattttttaaaattttaatttagaaaaaatatattcctcttgtataaatgagaaaactaaaggaaaagagtatcgaaaggaaaacattatagaaatatcagaatttagaatcccaattcttaagattttttattttctgactacttcaaataatatcgtttgcctttctcattattcatgggtcttccaaatatagtgtctggccttaaaagttgtaacacaaaatcttctgtctgaaatgtagtacaactgaatgaaatttaaaaccttcacgtaatatatgtcatatagaactctcataaaatatcagtatgatgtgataacaaaggtcagaacaaatggaagaaaaaccaatgtatgatttctgaaaaccttatcctgggaaatgagaaacagactataattctttacttacatctacattatgcaaaagtatctttgatgtgaaaaaatactcagtgttgttcattcgatgttttatatgttttggtaaaacattttgtgttcacccttttaaatattccaagacacactcactggctagaatcaatgggagagaaagaggtgactctataactaatataatgttaaggaaactgcacatatcttggtatgtttctgatagtgcataattccagaaaatgtcttaagcattgcataattgttagaaacatgaaatctgcagccagcaagcatgagttccacatctgaccctactctttccagttgtgagaacttatttgtattttaaactactgaatttcaggatgcctctgtttattcctgtttctttattttttaagttgacagtatttgtacatattcatggggtacatagtgatatttcaatacatataacatatactgatcagatcagggttattagcacacccatcatctcaaacatttagcatttctttgtattgggaacattcaatatcctcctcctacctatttgaagttctataatattgttaagtatagtcatcctacagtgctatagaagctataacttatttctcctgtctagctgtaattttgtatcctttaacaaatctctccctatcttccccagcctctagtatcctttgttccattttttacttcttcctcatgttttaaagggaaatgattacagtatctatttcattaggttgctataaggaataaatggtttcatagctcttaaaatagtgcttgacaaaagtaggtactatgtgtagatttttattaaaaaaagtagatactatgtgttattattatgactaatcttctcccaaaaatatgtttggtaaatcatttcataaatttgtttattcttaatttattttgaattagaatcatttgtataacaagttggtatattgctgagcacatctgtaaagtaagacctgaatatggatttgataattgaagttgacactcatcatagagatttatgaaatattgtgcgcttgattgcagtaggacaacttactatggggaaggaatcttgtaagtatcttgcctggccagtatacatggaagttggtacacatggaagatataccatatgtatatatcccgatattgataatgttactttccaaaagtggaggcactgcaaatatgatgagaatgtgactagagcacgcacttgtttgttctttctgtccagcattaatttcccccttattccagtaatagtaactcaacaactctgctctttgtcatttcctggtgtttttttcttttcttttctttcttttttttttttttttttttttttgagactgagtttcactctgttgcccaggctggagtgcagtggcatgatcttggcttactgcaaactccgtctcctgggttcaagcgattctcctgcctcagcctccccagtagctgggattgcaggtgcccatcaccatgcccggctaattttttctattttttagtagagatggggtttcatcatgttggccaggctggtcttgatctcctggcctcaagcaatctgcctgcttcggcctcccagagtgctgggattacaggcatgagccaccgggacttgtctgtcgttcgttccttccttccttccttccttccttccttccttccttccttccttccttccttccttccttcctccctgtctgtctttctttctctttctttctttctttctttctttctttctttctttctttctttctttctttcttttctttctttctttctttcttcctctcctttctttccttccttccttccttccttccttccttccttccttccttccttcctttctctctctctctctctctctctctttctttctttgtcttgctctatctcccaggctggagtgcagtggtgtgatcatagctcactgcagcctcaaatgcctggtctcaagggatcctcctgccctgatctcccaaaatgctgggattacaagcaaaagcaaccttcccagctccaactccacttttcagaaatgcttctgtaccacaatgataaccctgactactcataggtttcaaatgaggactcttccgtgtttcaaggctacagcatgtggcttaggagaggccaatcaaagacggcattaccctggacagagtaattacttcaggatgggcatgtgacccaccaggtgtcagtgagatgcaatgagtttttcggagtctttcttcccactgaattgaggtctggaaggttgggaggctagagctgctacagcatctagctgtagggaagctacggctaagagaagaatctttaggaaacttatcactatgaaagtcagaacaaaaggatacagcataactatgagctggtaacatcagctgagctcttgaatcatcatatgcctaagactagccctggacaaaccatgaaccaataaactcctgtctttatttattttttacttcaattagtttgagacatctttttctcttacaatcaaaagttatctgctaaagtgactaaaatattattttgggctacatcttccccttgctcatttccaacaaacacattttcttcttttctccaaatcctttattttagattcagtgggtacctgtgcaggtttgttatgatggtatattgcatgatgccaaagtttgagggcacgattgaacccaccacccaggtagcaagcatagtacccaataggtagtttttcaacacttgcttcccccaccctatacccacttatagtccacggtgtctcttgttcccatgtttatgtccatgtgtacccaatgtttagctcccacttataagtgagaagatgtggtattttgttttctgtttttgcccaacaaatacattttccaatgaaaatgatgaagccagaaagtatgaaatacattgatttaacaattagaactgttctaattataattgataccggtgcctgttttctttttatgattgagtaatgatcacagatttttgctatcagattttgagtttgaatcctgcctgtatgatcttaggagacttttcctaatctcagtgtatgtccttacttcagtttgcaaataaggataatgaaagcacttatccaatagggttgtgtggaggatgaaataatacagggcaaatgtcttagccaagtgactggcacttagtaagtcctcaaatcttcactatgatattaattgaaagcctagcattcacaatctcactggtttatcagttcttttttctggttgctttttagtgacttaaatcagagataaataagattggtgtctcactgggttaatattttttctggagagagttaacttgtgtggcacaggccatctctttattgaattagcaaagaactacattggatttaagattatctggggctgaggctaatatcaaatcctactctatgtttgtagttccttggctacaagcctacatacggtaattgctgtttaatatgaatttttatttttaacccttacatatagtaagggttaattgctatttaatatgagtttttatttttaacccttccctatttacgtcatgctttgcttaaggaccaagatatattctgagaaatgtgtcattaggtgatttttgtcattgtgtgtacctcatagagtgtacgtaaaacctagatggtagagcctattacacaactaggttacatggtatagcctattgttcctaggctacaaacctatactggatgttactgtactgaatactacaggcaattgtaatgcaatggtatttgtgtatctaaacacgtctaaacatagacaaggtacagtaaaaaatatggtattataatcttatgcgaccactgtcatatatgtgatctgtcattgaccaaaaatgccattaagcagtgcactactgtactttctctttactttaaatttgaattctttatatgggttgaatgactttctgacatagcaaataaaaagcatgaggagaagcattatctgttaacaaaattaacacttaaaatcaacaaagttttaatgtttcgttccaagaaaagcctgtggaagatcagttccacaactgagagctttgggctgcttcagacatatgtctgtgtgtacgctgtgaaggtgtttctcttcacagttccccgccctctagtggtagttacaataatgccattttgtagtccctgtacaggaaatgcctcttcttacttcagttaccagaatccttttacaggaagttaggtgtggtctttgaaggagaattaaaaaaaaaaaaaaaaaaaaaaaaaaaagatttttttttttttaaagcatgatggaattttagctgcagtcttcttggtgccagcttatcaatcccaaactctgggtgtaaaagattctacaggggtaatgttttattattcttattatgcttattctctgtgatgcttctctacctttacagtagtagaatccttggggaaatctgcagagggaccactttcattttgaagctgctggctgcatgttttagcatgtctcttctattagagaatccaggcatggcagtttcctcccccagtgtgcaaggaccatcttcatgcctatgtctgtcgctaggcatgagggtctctaggaatgggtgaaaaaaatgagggatgttttggaggcactataatactggggagggcagtctgctagctggtagctgaaaggtcctggtttacttcaacattttttttaaataaaactgtgcagtagtttttgttattttaggtttccctctgttttatctggtgtatgctgcagaagtgaactgcataacacatttcactcttagaaaatgcatttcatatatttaaatgacaactctgactcctatagtatgggggtgaaatagattgattttttgaaaagatggaaaaaatagattaagtgtcatttaggagtcaagatgttgattgtattgtttcaaatagcatagaaactcctgccactggttcagaggtaacatttggaatgactgatgtgctcttttgagttatctactttagtctggtgaaatatgatttgaataccagtaaaaatctatttaggttattgcaaagactcatataataagagtttgcttgaaaagataaactatcgtaaacttattattttttctcaagtccaaattctgatatggacactaagactgtgatgctacgtttttatgaaatgggcataatttgaggaaattggctatctctacagtatttcctaaggtactgcaaatgtgttaatttaagaagcaattacactagctagcagcaggttgctaagccagggggtccgagctacagacctctgatgtggatgacttcctgcccggtttggcaggagctgggtcccttcctgtgcagcttccgagcagttccaaacacaaccaggcatcttctgctccttcccactccctttctttcaagcaggatctacctatgtttccatagcctggcattttaggatttccagagatttttacccagtgaattgacttggaataaactcttaaatgtgataaagtgagccaaagggcacgtgcaaagacttggagagatatgtgcaagatcctcagctcaggttctacaatttaatatgatgtttcatctcttgttttggatctctgctattttttcagaaagaatgctgaaaaagatggctgccaaaatattcacacaagtgaaacagcaggcacaaaccagtatcctccatttctgagctctgaattgcatattttcccacaagtagcacaatcacttaaaacattttgggagtgggagattagattacctcatagacacctactgaatgtgagagatagatctccatgtgagcaaatggatgcacatatgagattccctgaataccaaatacagctttctttgaaaatattatagctatctgagtttctatgctgtatctcagtgctcagtgtcatgtaactgacacaatcaccttttattctaatggtcattgctttttttccctccctgtttctgtagcactttcttatgcaaggagctaaacagtgattaaaggagcaggatgaaaagatggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaagtgagttcttagtcaagttgccttcactgcctatttactaattggttctgggctagtcccagggatgatggtgaagaaggctggcctccttccctctgtctaaagtatcactaagatgctggatgggcctgaccgtgtaatggaccaatgatcctagaagtcttttggaagcactcatttgaacctgcatttgtgagacaggcagagaactggtgaggcatcctccagcgcgggaattaaggaaggacaaaagcctattcaccttcttgaatacaaattatatgcttaaaccagtgtaaattgaccctgattccctaataatgttgagaagcaaaaactgtaaactaggagtctatttaaattttattttttatatttgcaggagtagtatctaaattcctctttatagtctctagctctccataagtcactttgatcttcagtgggtttaattattcctttataccatactttctcctttctattgctctccacagaaggaataatagcaggtgacttgtaggtgccaaataagattctgagcaaagaacacacctggaaaaccttgaagttctcatgagaaaattttctaaccaaaaaaaaaaatcaaagcctcaattttgtgctttatgtgaattataaatgcggttttaaaatacttacattaaaacttgataaagttgctaagaattcctatggcattgatcacaaattttcttaataatcctcatgtcatttatcaaatttaggaaagtttatagtgctcagaaaaaaaaagcatctatcttcatgtcatatgatggtaattattatgttatacactattttacagggcaatatttataaataatggttttacttttctcttaaaatattcttaatatatattctaagttttattttatgtgttgtgttttctttttcagacgtttatagtattgaataaagggaaagcaatctctcgattcagtgccacccctgccctttacattttaactcccttcaaccctattagaaaattagctattaagattttggtacattcatatcctttttcaaatcgtcacttaatatgattttcttctttgaccaagttattgagctacacattttccaaaatatctgtggttggcaatgttatgtgttctttctttttctttccttttactcaatcgttagcatgttgcaaaatgagatcacaggtaagtgaattactttcccccgtcttctaagtgtttcttctctacccaactcactattacttctttcttctcttttcttctcccttacgaattgcttgccacatcccaagcctttctcattaattttgaccatgttaccaggctttcctcctgtaagtcttcaatttacaatgttaggtaagggagtaaactccatgagctaattttctttactgcttttacatttggaaaataaatatacataatctgaattacaattttgtatgtttttggtctgaattttatgactttcttctattttagcatttaaaagctttgagttagtaagcgtttacaattgtgtctgtaggtataacaccctttcaaatacttttccaaatttgtttcgcaacagccttcttattgatcttttgctttctattcttttcctttctttttctttttatcacttgttcctatgttttattgaagtcacaagtcttgctacaattatccctctccaaaggattaatgtctatctatccatatacattctcattttattttttatactccttattgaagcacctgctttccaaaaatgagattgatgacatcttggtgggagatggcaatttgattgattccttgaaattaaatagagtagttggaaatgagagattttattctaggccagaaacaagtcttgttgacagccagtcttgggaatgccaatgaagcaaaggcttggaagactgaagctgtgtggggcaggggcatttacacgaagaacacagaagtcatgggaggaggagtgatatgcttcaggaatcagaggtaaggcaggggaactgaaattaagcaaatcctcagattaggacagcatgttatttcttcttcagaaagaatcgttgcttggaattccatgatgtggttagcccagggcctggtattaaggctttcagtataaatattctccacctttagcaggcctagaaaatatttgaattagataaggtatggaactaaattaagtagggaattcaggaaatgggataagcctggtacagggtacttatgtcatttctgggtggaggggatgtaaagcatgtccctagacttgcctcccagtatggcaaatgttgtctttagaagtgtaacattctgtaaagtctccttttaacctctaggttgttcctcttccccagtttaggtggatatcaacatcttttatttgatgtttatgtttcatgttttaagatttcctagtttcttggcattaccttaagcaataatgttttcttacctctctctgttttccaaataagagaacccagtagcatgtggggaaaaagatgtctttgagttagcattagaaataaataataaagttggaatttatatttgggtctcatgattataaattatgatctattattatgtttcaagcatttgtaatctgtgcagtgaataaatctctgcataaactattcattatattttaaaataattgtatgttccttatgcaaacgtaatttatatataaaattacgtggaaaattctagcctagaactagacttctgttcctagtagacattgggaaaatattcattaaataaataagtgactagtaagtcagagattagagaatcagatacaaaaaaagtgaaaaaataagtttgaatggatcagaaaaaatctttcttgtcgtgcatctgaatgatgagatggagttaagaaaacccaatatatttgttttttacaaaagcagatttttgttttaaaacttttgtaatagaccatggaaaatctcatgaaaactattgtccccacttgaaaaaaaaaatcctaggagattatgaatccccatttaaaactccctggaaaagagactcctggtggtagagggtaagggcagtttaagaaattctgatcagagaatatgagtactaaggacacagtttgtgccaggacctctctagatatccagatacaacttgaatctgtgggcttgtatttgcttcctgggggaaagaactcaccctccacaggctgaagtcattgggaggcctgaggagatgcaggttgaaatgtgaagccagccaggcaaatctattaaggtatgccaagttgagctgttgattctctgagaagtttacagatggcttagagtcacaaatttattccaatgtagaaaattagattttaaaaagtctctaatttcctgacttaaagtgttatatttcagatgtctcaccttggagcagagatataacaaaaggcagtgaggctaatagtctaagatacatgaatccttccatgttttggtgatgctggtgcaattgatcaaatagcccagtaaagttagaggtatatagatgctgtagttagtaactgattttcacaataattttgtcctttattcctcttgttgcaaaccctagacttaaatcctgattttctgacttcaagtacagtgtcttttactgtaagttaaaaatgcttggagagatggtcatggttgtttggccacagttgggaggtcattgtatatttattaccactagtttataaaccaacaaggagccattcatgttaaataagtttttattttaaacttggactaatacctctatttcaaacaaaaaccttgacttgtttctcaaagagctgttatctattaggagctattgtgtatcaaattagcttttttaaaaatttattttggctgaatgagaaattatgcttgtgatatttttaccagggtgcattctgaaaactgaaaattcttttgatgtgcctagtgtcttatttgatatttaaataaaacatgatttattttctagataacaaacaagttaaaaataatctatgttcctaaagttccctaccaagcttttaaatgtgtttcctgtcagcttttattattttaagttaatatatgcacactcctctaatttattttgcatttgttactcatttgttcatttgcaagtacttactgagtatctaccatgtggtagatattcttgtaagcattgggatgcaacattgaacaaagtgaagttcctactctcatgtagttacattcatgtgagtgtgtgtgtgtgtttggaagaagaaagacaataaacaaatacgtcgattgggagctagtgataagtgttactaagaaatataaattagtgtcaaagggaggagtgacaaggtgttgttttagattgtatggccagtaaaatcctttctgaaaaggtaccagttgagcagagatccgaaggaagcaaaagagtgagatatgggaatctagggataaagtcagttcaaggagaacagcaagtatgaaggtaaagtctgaaggtggtggtgcaactagtatgtttaacaatcagcaaggagacctgagtggctggagcagagtgggaaaggcaggaagcgagaggatgacatcagtgggagtgaaggtcaggggctaaagttgtaggcgaggtgcagtgggtcattgtgaggacaccacatttactctgaatgagactccaggagggttttcagtagcaggatatcatgacttgactcacatatttaaaagatcactctggctgcttcatggagaatagactgcataaggggaaagagtggaagcaaggagactgggaccgattgtcattatgaaggcaagagagcatggtggcttggagtatggtggaaacaatgaggatggtaaaatgtcatcaaattttgaggtattttgcagaagagctgacaggatttgctgagcaattggttgtggtgtataggaggaaggcagggactaaggatgattccaagcatttagtctggtcaagaaagaaaaatggagttgtgttgactgagcttggggcaacttgagcaaaccaatttggtgggtaagatcaagatcttggttttgaacatgttatgtttgagacacctattagacatcattggaggagttgaggagttaggtggctgtgcaaatctggagttcagaaaagggctgggctggaggtgttcaactttgggagctgtcagtgtacagctggtatttaaagtcatgacattggactaggctgccaaagagctgagaccctccctccaaatcacactagtaatgctgaactacctatcatttgaaaatggtaggaaaatggaaaacataggttttggtatcagaaaacgtagattcaaaccctatctctaaaatttactttttagctatatgatcttagtccaagttactccaattctttcgaatctcagtttctctatctgtaaaattataatcacagcttagacattaataatgataaaatgtatgacaagtatctagcaccagatcccatgctagtacttagtaggtactcaataaaggatatctatgacagtaatagctaaaattctagcagcaactgctgtaagattagcaaaaaggaaactctcatattccttaaggaattgcacaaagaactttatagaaatccctactctgactctgcaaacaaaatctttatatagcaccagagtttagacctgcaactgacccaaacaatgtggtcagttctgtctcattttgtagatgagttcactgaaacccagagatatttagttttttctaaggctacattttctatcagtggcagagctaaaacttcagaccaggttttttgattcttggctctttgcattttgcatccaatagaaaacaaatgatttttaaaccctcggatttaatatacttggggcattgccagtgttcttgttttatgcatttcaaaggtgcttcttagttgctccaacttactgattcattaaatagtgtccatactgagatataaaatatcatggttttccatgaaaagaaatatacaggtttatatgaaagcagatgacacaacaatttctctttcttttgttttcaatgctcatatgttatcatttagttatctactggcaaataggagtttgttcatattaaaattaaacaatccaatatttaacactgtatatgtgacatttactcgatttttctgctggctcagaaatatgcactggtatgcagaaaaagacctattctattctacttcaaattatccatttttacattagaaaacctctaacatcaggctatcttctacttctagtttatatataggttaaaaactcctctgcaacttctctggatattatacattattacaaagtctctgaacagagcataatgtcttttccttcctatagaataacaaagaaatgtcctataattttatactctataaatgagttattaatggtaagaaaccaataattattatcttagtggataatgactgtatactgtaagaaaagtattatccacatttatataagaaaactgagcctcaaagaattaaacaaattgctgaagcccacatggctggtaagggatgtatctgaccatggttcattgctctaaatctcatggtgcttcatcctcgctccacggagacaggggtgggtgtgccagtgttatgatgatccaggctccatgtcaagggctacttaaacaattttcactaaaaacttgaagaagtgtttcttcataatatacacaaaggaaatattttacatttgccaactcgcaggttagtatcaatcaacaggtttacccactgttatgtatacctggcataaagaaattaatagattaaaaaacatctttgtcccctgatattataaaaggtttatctgcctctattttattttacattgaaaagttcttaaagcaatattgttccaggatacagtgttcttttgaaaaatgtactctatgacttggattacacatttaaaaaataatataggatgtatgcattttgctactagtttgagccttttgaaatctgctttgacgtggggtttctatacttttttgatgcatggcatcaccaatgcaaaatccatacctacattaaatacttttgtatttgagtttttgttatttgagtttttttttttttttttttttttgagacgggttctcgctctgtcgccctggctggagtgcagcagcgcaatctcggctcactgcaagctccgccttccgggttcacgccattctcctgcctcagcctccggagtagctgggactacaggcgcccgccaccacgcccggctgattttttgtaattttagtagagacggtatttcaccgtgttagccagggtggtctcgatctcctgacctcgtgatccgcccgcctcggcctcccaaagtgctgggattacaggcgtgagccaccgcgcctggccatatttgagtatttttaagatcatctgaaactatttcagtcactcaccagaatccaggaatttgtaaagtatgtgactgatgaaataaattaacaatgatttagaaacttagtgaattttaagcctttctatttagagatacctatcaaaccacaagcgtaaaaacttgaccctagttatctactatttttctattaaaagcaaaattgttctttttatgtatcagaagttttaacttaagtgtatacttttattaaaatgatagccatgaaataaggaaaatgcctgttttcgacttattatcagtgactaattagaaaataattatttctcttgttaatgttgaaatatatattttacttttttatatataactaaattataccactataaagagtaagtttttaagtgtcataaaaccattgccgagtccataatgcagcataattgcataaggctgttaatttccaccttatatttttcttatatttttaccctcaaaaaatgtagaaacttgtgtaaacaatatgtatatatattttagacagagtctcactctgtcacccaggctggagtgcaatggcgtgatcttggctcactgctacctctgcctcctgggttcaagtgattcccctgccttagcctcctgattagctgggattacaggcatccgtcacgcctggctaatttttgtatttttagtagagacagagtttcaccattttacccgggctggtctcaaactcctaacctcaagtgatctgcctgcctcagcctcctgaaatgctgagattataggcgagagccatggcacctggccaacaatatatttgaagacaaactttatgctgtatttttaaataatttatcagaaattgtttttaaaaactccatttagtaacaaatgaattgcaaaattaatttcattagtcaactgacactgtgaaatagcaaggctataatggtgaataatatagacatgattcctgtcctcatgacgctcagagagtagttgagaagatcatcattaaaatttgtcattagaggaataatataagggctgctggggtatataactggtaataggtcaatctgagtactaaagaaaaagagaggtgacatttccaagaccctaaagtcagaaacagcatataaaacattcataacatttgacaacctaaaataattacagtattatccaaatggggataatgcaatgaagagaaaagaaaatgaaactggagaagtagacagggatcagacctcctagcgtcttgactctgtgttaagacatttgatcatcatcctaagagtaatagaaagctaccaaaatgatgcatattacatttacaatggtcgtgttagcacagtatgcagaatggattaaatggagccaaacatgaatttggaaacatcagtttagaggagactgcaataatctagatggactattagatttgatgctagtcttgacagacacttggaccatgcagtgatatggggatggaaaaaagtaactgaatccagagatagcaggcagaattgacaatatttgatggttaattagatatgaacgtttaaggggagacagaaatctaagatttctcataggtctctggctatattatgcacattttataagacacagagacgtcaaaggagtaagtaattagcaggaatggaggggtagattaaaagatacttttcaaaagttcagttttagaattcaaaatttgaagtgttgataagatatgtaagtacagatgtcctatggacaatcaagtatgtggaattcagaagagcggtctcacttggagagaagtatctgagaatggtgggtatataatggttatgtttgttgagcaatgtttgttgatggactacactaggatgaggagagtagaagagaggtagatggcttacataccttatgtctttcttttcaaaaagaaaaatgccacatttcaaagaatacagagaaattggtgccctgaggcatgaagaagccagggaattggaacctcctgaaagcagagggaagataacttaatgctaaggggaggaaccatccttgttgaatgctgcttagaaagcatgtcaaatataatctcaaattatccttttgtttttagtgacaaaacgtgaagatgttgccatctttagaaagaaaagctggtaggctgaatatatgatagatatataagaaaaggaaagataagcagctctttcaaaaagtttggccatgatatggaaaagggagataaggctgtagtagttaaggagggttgtgagtcataggagagaaatatcctttcccctatgccaggacaagggagacgtaagcaagtttgtgttgctgggaaaaagccagtaaggtgggagccattaaggataagaaaagagagagataagagattgctgaagtcttctggaatggggaaggtgtccagaataccggtggagggattggaaacctccccactgtaacaggaaggaggaaagaattggtctcaatgtggaaaagtttgttgatttgggggtgggaagtggaggcggggcattgtgatgctgtctcttctctgtaaagtagaaaataagttttcagcttgaaatggagccggaagaaagaagagggttggaagctggaggaaagtggagaatatttgaaatttttctttgcagagagtgggagatggagcctagtaggaaaatacaggactgtgttgaggaccactgaggtttgtgaccataaatttagaatggtgccaatctgccacggggtgttatttttccccaatagggctcagcagaccaacaagcacaggggaccctctagttttatataccaatagcaagtcattctttatttaatttagttttttgtttgttatagcaataaagaaaaattgtgtttctttgaaatggtattttgtgtgtgtgtgtgtgtgtgtgtgtgtgtgtgtgtgtgtgtgtgtgtgttctctagattatagtgccctcacgtggctgatgcacatcatttacttatgagtgttttcatatggatgaaaaaactgagaattaagcatgggccttacagcacagctactaaaattaaaaaataataattattattattgttttgactaaaaccagtactagatgcaaatgaactttctttctagatcaacacaacattgtccagttgtaatagtgttgatatttcattatgtgtaagtaatgtgatcatttatagtaaaaacattaggagtgagaaaagatatgaagagcacgtatttcctctctggaatttctataattgtgtccagattcacaatgataaagagtgccactttacataatggcaactaaatctttattatgctttttattaaatatgaaagtcattactattatctgatactgaatattttcttaaatagttgttttggtttttgtcttttgctgttttttaatgtcaaggaaagtgaagggcattggctatacctgcaaagacaaagtgggatagggtgaaaccaacccaatatttgtaataaactgtgttctgtatgatccaggaagcaattcattgagcatctattacgcactagggccattaagttgaaggagacttacatattttaacaaatttgatcttcatagcaatcctgttacatgattactttttcttacttttataaccaagtcaattgagattcagggaacttaataagtaattttgccttagttacaaaccccagaaatgccagtgctaacacctgccatcttctctcatagttcaggattttatgagcaattacagtatattataccctctgtttagaaaggaccttattataagacattccaccagggtaacttttagaatgatgttataatacatttaattaattacttgaattgtcttgttgaatttttgccagggtttacatatgtgctgaacttgcagttttaatgttcagttgagtctgtcgttaagaaaatttaagttgataaattattcactgatgaactactttctttgcatttaatctttttaattgctaaaggtacctaaatagcctcaaaatagttgatggcttggcctgaagacaagatctaaatatgaggttgctgagttatagaaatggcaaaaaaaagggtcaataatagaataataagcaacaaaataatagtaagcactaaagttttaaacttcatggtggtgaaggcatggtagtgcataaaagtaagatttttccattgaactttgtcttccttgacgatattctactttattcaatatgctcattatgtgcacgattcttaccaactgtgtatttatgaccatgagtaaccctccagactggacaaagaatgtggagtaagtataaatatttttcaatattgacctccctttatgtttcatattgtgcttttaacaccttgagacctcctcaatttctttaacaaatcatgctagctactgttaaccagaccctgattcaaattcatttctgtcactaaatgtcttctaggacaaagcttgtagtgggctcacttagttgtgtaaattactgcagtagtttgactgctattatctgcagccctttatcttctttgtgagtcttatgttcttttgaagatcaccagtgatttactaatatctactgataaaaagtatacctagtttttatgttccttttttaatgactaccacagttctgtgttactagtatatgtttgatggcttttaatggtgcatattttatgaaatacaaatgcttcactcatttttgtattaatacctatttgctcaaatcggactgaatgccagtgtatttcagattatgtcttcatatagagccacattatttggatcctttaaattaattaatgtggaaaatgcaatatacatttatttacagtcaatgagaatgtcttttggaatttaatgtttcttttttgacttaagccccacctaaactctatatcgtagggggaccaacctggaagtgtctaatttttgtttgctgtttatgtcatctttaagatatgtacttgtaaattaaccactagatttttaatgtgagcttggctattttctctcaggtatacctttacaggaatttatacttttgaatcacttattaaaatacttgcaaggggcttttgtttagaagatttcacatttttacgggatccatggaattggttggatttcacagtcattacttttgcgtaagtatcttaatacattttctatcctggaagagtaaatcactggtgggagcctatactatattttccttggtggcttgccttgacagaccaagcatttttcttagtaatcatagttttcttccaatcaaattatccagtttggagaaattaggaactatcatagtaaattacatggctttggtttcaattagcactgtaaagtaataaagtttcccaaataacagagattatgattgatgacaatgccattttcctcttaattgggaaagctgatggcgacactcatgaaattaaaaaggtcttgatgaaagaccaaggaagacgtagatttccctaaattctgaataactctgatttaattctacaggtatgtaacagaatttgtaaacctaggcaatgtttcagctcttcgaactttcagagtcttgagagctttgaaaactatttctgtaattccaggtaagaagaaaatggtataaggtggtaggccccttatatctccaactgtttcttgtgttctgtcattgtgtttgtgtgtgaaccccctattacagatatgtgacagagtttgtggacctgggcaatgtctcagcgttgagaacattcagagttctccgagcattgaaaacaatttcagtcattccaggtgagagctaggttaaacaccgaggctgactttagctacagtggtgctacaatcacagcttttgtgcagaagccttgttgctagttgcatattgcaaataaatatgtaaaaaagcaagaattggtacatcattttttggatggatttgattctttgctttttactcgttgctttctttaaaactattctaaatcagcctttgagtttaacaagtgttgcatgaggcatttgcagtaacaggctacatggtttgcatcctataacatcaagctttccgcatagaagctagactaagagacattcagactgatgcaaatttgacagtttaggcctaaaactggcaatcttttaagctgcagataaatgaaagagcaagggatagcatgagtgctgcatggggctcagatttcagatgtcttcctttttttaacccatactcaagcttgcagaattcacaaatatataacctcataattcatcgacttcaagatttcttactactctattcacatagacttttctaaaaccaataaggggttagggagtaagacatctgcaaataaaagcaaaatatttacacaaggttgatgtttaagcatgaataacaaaatcattcttttgctctaaagagtgtttggaaatacacatttggttcatttccattcacagttttctaatgaacatacaagttctgctttcattcattttcaccagctagcaggcttttcatgaaaatgttattcaatcacaaacattaaactaatattgttggcattctgcatgacatttttattttccaggacaagctcatgatatttttgccggtaaaatagctgttgagtagtatatttaaattcccccttctgattttgtttgtaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtaagatatactctataaaccattaagttgtttagttctctaaatattaaatattatatataatggaaattatctcaatttagatgtgaatcaagtgacttagactaatttaagatgatttaatacatataaaagagatatcaaatgataccttattctatttttcttatctgtccattgatatagtaaaagttctcatttgaaaatgtgttgtcttatactcatgttgaaagtaatttcatattatgccatattaaaaaatgtttatttggtagacattaatcaggtttttcagtcattttaataaataagtcagtagtttgaactattcagtgtattccactgaaatgtgttaagaagactgaggggaaataatttggccctatttggttgatgcaacatatgtattgagtacatatgctatatctgaaaatagagaaaccatttatcaagatgaaataagaatttgtgtgctcctcagaaggttaagtaaccctgatttagccattcacttattcatattctaattagtccctttagtgtcatcattgtattgtagttaccagtttagtttgattatatttaaggtatgaacatcagaataagcttatgccatatacttcagcatgatttcttaacattgagcccagcccctctgtcatttttcatatgtgtgtgcatgtttgtatgtgaatataaaaatacgtatgtttgcatgtgtgtgcatgttttctgagatcatctttgcaacttactgaagttatatgtcatgccttaaaaataaaaactagatagctctccatagcttaaaaataaaaactagatatactcagacaacatatctctccaaagaaacaagtttattttcttcatttgaaaggcagaaatcaagcaaaaatttcaaacaaaacacttatttacagtatcataagagggaataaatacctaatcccacttctcacaggaaattaagttaaaattggcgggaaaaaatgtctgaatctattttgagcctggggagaaaagtatatgtaaggtaaaatttatttgcatgaaaacacctagaaacaacaaggctttcttctttcttactttttgtgcccagcaatagactggcagctctttcttaatgtatcccatgcaatttgagcttatatttgcaatgaatgctgatataagaatgttatcatagtaattccttctgaacatttttctttttaacatagatttgctaaccatttgtataatcaaaaatgttatatattgatatttgttcaatattgtgaaaaatctctttagccatatatatttattagtttatccatctcattatgattgaaaacatttgtgagctttgccacctaaacagggtggctgaagtgttttacaggattttaatgattctttctattcctttctctttaaataggtcacttttattttttagaggggcaaaatgatgctctgctttgtggcaacagctcagatgcagggtaagtgatgcttcctactgagtttcagtccacactgctccatcagtgtcaataacctgccacctcccactcatccagtcccactcactcctcactcaaaaccctccataaattctacttcacggtgactctcagaatagccaggataagtgtagattctcacctctttcacacagtcatttactgcaattatttttctatgctaggtcacatctaatcttccaaattagttcaatgtaaaatagagaataaagcagtataatatgcatctgaagcttaatagaattcttaagcacatactttttataagtgtcatattttatatatactaatgtgttctccatagcttaaaatgtaagatctctgaaaataatgttaatatctgagacatggggagtatttagcatattttagcaaagtggttacaaacataaactggagagtctgcatagagtcagactttgactccatcatataatctcatttcttcttgccttcgtttccttatatgacaaatgggtataataatagggtttttgtgatgatgaagtggattaataaatgtatagctatttagatcgctcaataagtgcttgtaattgttattattgggatcatgcaaatgtttgctattaagaaacatggagctaaatcctaggaaaatttaaaaacacagttaattttctttatttagcaagattttagagccacacacaaaagtctaatgcactttctttggacgatgatactgtggacattagtagctaatacctgtagcaaaattcccagtgataataggctttccatttggctcctacgatcagtgctatgctgcctttatcttcagattccaatgataagtaaatcaattgattttcattccttgtttgtactgtactaaatgcgttacatacagtatcttcttcaatgtttgcaaatttgtgagacaggttctcttattagcccattctcacatgcgaggtgcctgaagattagcaagttaagtaacttgcccaagatcgttcagctcagaagtgtcaggcaagacattgaagccaggtctgcttgatcttcaaggtcctcctatgacatttttaccacacagtgtcattcactccttgcagcatgccccacctatccttttctcacttctttaccctgttcccacacttacacacatttctgcctcaagacatcctcagtgaaaatcaactttttccttacagacttttttaactgcccttaagtcccagaagatattaatcatgatatgattgcttttatatggagacataataaatataataatgacaattatgaatcacagaggaatccacaaagtagaccttatagattctgttattatataaatcagtccacttagtgctgagttaagtactgggtaaggtgagagaaatcggcttttttctagtgcctgtataaaacagacattggcatatattaaaacaggaaaaccaattagcagacttgccgttattgacttcctttctttcctctaacctaattatagccagtgtcctgaaggatacatctgtgtgaaggctggtagaaaccccaactatggctacacgagctttgacacctttagttgggcctttttgtccttatttcgtctcatgactcaagacttctgggaaaacctttatcaactggtgagaacagataaaatcatttttctgagaatcataaaacaccgaactcaagagaattgctgtagaatattttattacttagagtgtaagtttgtaacatcctatataaaatttattaaaatctctcttccattttgcagacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggtatagtgaacaagcatacggtcctttgtttttctttatctaaattctttaacctaaatgttgaggtcagtggcaaggtagttgacattagaaataggtcatatgtgtttggtaagtgctaggagcctgtttggttattaagaagttattactttattgcaatgatctctgtcaatagtgtcaatagtaatggcatcaaaaaatggataattataattgctttactgacatttttttctcccttgtgactccttgaggaaattaatgattaacaaaggcctcatgtactcaaacttgcagagtagataaacctacatgtcctcagttgaagtattttcttaggggaagaggaattcagttacacttgcttcttcattgcagtatcaccagaggtggtaagggtcagaaaaccagaatcaaactaagaaaattatttcattgagtctggaaaggcaaaggcttattcaatatttgttctcttttatataaagtgtacaaatgcaagtttgtgggttacatcagtaaatcactagtgtgtaaacatattaaaacattagcactctctgcctcctactctacaaatcctttaatttggacttgacaagccttcaaaataaggcaagaatttctctaattatatttgcttgacttaatggcattaactaatccaattgcctatttttgtcttttcatgtatggtgaatacaattcccttttattaccgagtattcctaaatatgtaataaaggtcaaagtatattgctgtaatagcaacaaaactactgttatactttacaagttcatgcagatgccatgatctaggattctcaaataaacactctgtattatgtctttgctgtgcatttcttagtgaaatacccaatttaaatcacggagaaaaatgtcattaaaataaaatacttgactgaattacatttaataattcagactagcactaaatttctttattgtgtgaaaatggaatcaaaggcaaatgtctaccaggtttaaataggaagtctttaattcccatattatttccttcttaaaatattgtttgaattatagaacatgttattatgatctttaagtgtcttgctcatattattagataattagatatcatagtgtgaggacagagcttgaaggttctcataaaagtcgtatgtatcatcttccatatgaatgcccattttactctttgattggtctaataacaatgtactgttttctaaaacacagaataaaatggagaattgtttttcaagattatcttcatgatattgaagctcaattaagcagtaacatgataattactttttaagtttatatgcaacttccacatactttgcgcccttctaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccaggtaaaaatattaaattacatgaattgtgttctcataaattttttaaaaaaatatgccagaatttaatggagagaaaaccgccttccacctggatggcacaatgctttcagagtagtgatgattatcaagtgttttggctatcacttcagagaatttgtgagttttgcaactttttggaatcccaggaaggaaattttagatccctctgggtttggaaaaatttgcggttttgaggttttcttaaagactgaaaaatcttggagaaattttccacatcaggaattatcagcagatggttcccatctcttcttaactattgtgcgtggatctagtgaactttgggttttctgagtgacaaattcccagaagtggaccagagactcttttaggccacctgcggggttgttcccataaggtgcaaacatcacttgccaagtgcattcttcatgcctttgtttcaaaggggactgaaacaaaatatctctaaaagtagccaaaactctcagataggcaggtactgagggagatttatgacacgaaataaaaagtggtgtttagttgtacttgattatctgtgtttcatgttaaacatgggacttgcatttgaagaatactgtgatttataaactgcaacaaatattcactggatgcctctgccttttgtactcatgcaagttgttgaaattttaaaatttagaatcttaattgtctttgaaattaccaagagaattcacaggaatacacagtacctcagaagacattttcaccaggagtgaaaccttaatacctatacagtaacaataacaattacaacaacaacattgataatggctaatatttatacactgtgttgttatttgcaatattaatgcattaagcctttcactgcaaccctaggtaagttctaatttagttaatatccccaatttttatatgtgaagagaagcagggggctgcattctttgttgagtacctattttatgcttggcattctttctacattctcataattaatgctaacagttctgtagaatagtattattttcattactataatgaaaaagctattttggatcagtaaagttaaatagttgcacaatgtcatatagcaatagaaaaagttattatgatctttgagtgtcttgttcatattagggttgttggtattcaaattttgcctggtactgaaaaccttaaagtttccactttatcaagttgcctatgaagaatgcctttaaaaactgataaggaaatttacaatataactttatttaaaatacacaatggcattatactttctcttttacctttttataatatagacaagctcacataacctcacatgtgatatatataaatttttttaggtcagccttattcatttcaaatccaaataacatcataagattgcatacttggggattaattcaaatttaacataggatctttaaatatcaaaatttacttggtctctttcattttgttgtcacaatcatgattccattagtagaaacattaatcaataaataggaatcctttaaaaggcaaaccccctgtttacagtattagtcattctgaaaaggaaggaagaaaaagaaagggagggagggaggggggagagagagagagaggaaaaaagggagcaaaggagggaggaagatagagtatttttgcctacatttttacctaagtttgtctgaatttttgcctgaagttgtctaagttttggccaaaatttgcctaaattttggcctagatttgaaacttcatatcaacttcatatcaaacacttaccacagagattctcttcaatttgccttatttctaattgaataaaactaattctaggcaaaatagtgaagcctgataaggctaggctctgtccttcctttttctgtacattttgttcatagatatgatattctcccagcagcctcttcttcatacctctacatacctccatttcccagctagttgggtattataagcaatcactgacttagaagacatggcatggctggctcatattgatacttgtttcttaagcagtctctatataaaaatagagttaaagactttattttgcttgataaagaaatagtcaaacaaatgtctaagaggatggagagggagacagaaaaagacagagggaaagggaaagaaagagaaagagagaggggggaaaggagaaaaggaagaagagaggagagacagaaaaccctgaaatcacaccaaccccacttggcaagccctgaaagtaatactgaaaatgtcaaaccagatagaagtacttaatcttggtatagcaatggagggcccatcgtgtctgttagatttttaagagtttgagaccccaaaatattaggaattattgttttgtgatgacatgattgtgttggtaaccatctctgtgtttgtattgaaaaactatacaatgcaagcctttactgcaagattataatttctttagtagagtaagtggaaatatgaattgtttctcagactctgatttgacttgttagtgtggtaaagaggggagaagaaagtcaagaatgtaatctctaaacaagtttcaagataatctggatttttttgaaacctttataaggtacaattgaccttaaatcattactttattatttatttgtgataagctaggagtttaggagttttgctttttaaagattggtttggtatggggaatatttcttactggccatcttttttgtgtgttacagcatttgattactatgcatttatgtaatgaatgtcagcaaaagaagttgatgctaacggatggggcacaatcatttctcatatagctgtcacatgtaaactacgtttttgtatagcttaattcatccatgatccttgagaaacatgcaaacttataacttattttcttccaacccttctatggctccagctgaatggggtactggcagttaaaatataaactcttactaaaagcgatagaaacattcttcattgcaaagcatgtattgtttgcctttcttttttagctaatgaggagcagtatgtcacacatcctgcaaatcccgtaactgttatttcctcatagctaattcgaagtcccttgttagaggagagaaaggagacacgaaaaaggatggatagtctaagaaaggctttaaaaaataactacttgtatggaaaatgataaaagaaaagaatgaatgttactaatgtagttaataggattaaaaagcatgggaacaacaagaggagagatgacttctgttgtgggagcagtaagtcttcttagaagtagttctaggccgggtgtggtggctcatgcctgtaatcccagcattttgggaggccgagacgggcagatcatgagatcaggagatgagaccatcctggctaacacggtgaaaccccatctctactaaaaaatacaaaaaattagccaggcgtggtggcaggtgcctgtagtcccagctgctcgggaggctgaggcaggagaatggtgtgaacctgggaggtggagcttgcagtgagccgagatcacgccactgcactccagcctgggtgacagagtgagactccgtctcaaaaaataaataaataaaaaaaaagaagtggttcttactgtaaataatgaatagaatcacataagatagtgtttaacatttacagacatttaatagaaactaacagatattattgagaaaaagtaattctttagctggaaagaaaataaaaagcatacttattggtcagtgtattactctgttttcatgctgctgataaagttatacccaagactgggcaatttggcaaaggaagaggtttaattggacttacagttccatgtggctggcaaagcctcacaatcatggcagaaagcaaagaggagcaagccacaccttacatggatggtggcaagcaaagagagagtgaaagccaagcaaaagaagtttctccccatataaccaccagatctcatgagacccattcagtaccatgagaacagtatggggaaaacccctaccatgatttaactatctctgaccaggtccctcccacaacagtgggaattatgggaaatataactcaagatgagatctgggtggggacacagagaaatcatgtcattccacccccggcccctcccaaatctcatgtcctcacatttcaaaatgaatcatgccttcccaacagtcccccaaagtcttaactcatttcagcattaattcaaaagtccacagtccaaacaaagcctcatcggagacaaggcaagtcctttccatctatgagcctgaaaaatcaaaagcaagttagttacttcctagatacaatagggatacagacattagctaaatacagccattccaaatgggagaaattggccaaaacaaaggggctacatgccccatgcaagttcaaaatccagcagggcagtcaaatcttaaagctcaaaaatgatctcctttgactccatgtctcacatccaggttacgctgatgcaagaggtgggttcccttggtcttcagcagctccacccctgtcactttgcagggtacagcttccctcctgactacttccatgggctggcattgaatgtctgtggcttttccaggtacacggttcaagatgttggtggatctattattcttgggtctggaggacagtggctctcttctcacagctccagcaggcagtgccccagtagggaccctgtgttggggctctgaccccacatttcccttcctcactgccttggcagaggttctccatgagagccctgcccctgcagcaaacttctgcctgtacatccaggtgtttctatacatcctctgaaatctaggcagatgttcccaaatcccagttattgacttctgtgcactgacaggctcaacaccatgtggaagctgccaaggcttgaggcttgcaccctctgaagccatgggcctagctctacatttgcccctttcagccatggctggagccgcagagatacagggcaccaagttcctaggctgcacacagcgtgggactctggacccggcacatgaaaccactttttcctcctaggcctccgagcctgtgacgggaggggctgctgcaaagatctctgacatgccctggagacattttctccattgtcttggggattaacattcagctcctcattacttatgcaaatttctacagcctgcttgaatttctcttcaggaaatgggattttcttttctgtcacattgtcaggctgcaaattttccaaacttttatgttctgcttcccttatgaaactgaatacctttagcagtacctaagtcaccacttgaatgctttgctgcttagaaatttcttctgccagatactctaaatcatctctcccaagttcaaagttccacaaatctctaggacagggccaaaatgccaccagtctctttgctaaaatataacaagagtcacctttgctccatttcccaacaagttcctcatctccatgagagaccactttagcctggaccttattgtccatattgccatcaggcttttggtcaaagccattcaataagtctctagaaagttccaaactttctcacattttcctgtgttcttccgagccctccaaactgttccatcctctgcctgttatccagttccaaagctgcttccacatttttgggtatcttttcagcagcgtcccactcctggtatcaatttactgtattaatctgttttcatgctgctgataaagacatacctgagactgggcaatttaccaaagaaagacatttaattggacttacagttccatgtggctggggaagcctcacaatcatggcagaaggcaaagaggagcaagtcacatcttacatggatggtgtcaggcaaagagagagtgagagccaagtgaaagggatttctccccataaaatcattagatctcatgagacttattcactaccatgagaacagtatggagaaaactgccactatgattcaactatttcccaccatgtccctcccccaacaatggaaattatgggagatacaactcaagatgagatctgggtggggacacagccaaaccatattagtcaggcatataaaaacctagatattagtaggtataaaataatggttttagttagttttgaacctttggagaggaaaagatcaaaccaataatacttaaatgatgaagttagtattctttccaataataattacataaatccaatgcaagctggctgaagtaaaaaagagaatgtattagatgatataaattatgtctgtgagtagctttaggcatggatggatccagaagctcaagacatgttaccatgaccatgtctttctccatatctgaatctaggttcctcccacatggtggcaagatgtcactgcagctccaaacttaaagccaatatttttagcagcctcaagtgaaaaagcaccatttcctcaacagttgtaaaagacagggatatctttcaattgaccaagcaggatttcatgaccatctttaaattacgtatcgaatacactgattggctaggcctaggacagtgcctggaacactgggagacccgagcctcaagtcctgctgcatccaggagttgaaattagtgtctcccagataacctggtctgagagttgaggattatttctcaaggctgaacagggtactgatttaaaaaaacaaaaaattaaatggttgtgatcagcctcttagtgaaaattaagtttttgtaaatattgccctcagatttcttgagacagagacaaaggggtgaaaattggggaataaatcatacagttatttcagcttgatttaatttattcatgaagaccatcataaaatatgcaaagggaagtggagaagctgccccgtgtactataattaaacatccctactagcaagattaattatatttcctccatggtaagatttgcatcagggtgtggtcactagcgagctcttactggctacatttttgacctcagaggatctaaaggtagatttgtgtttaattgttttccattgggttgttaactgaaattaacttctaaagaagggtctatcaacagtatcagttctagatgcccgtaacaggacaaaacattatggggacacttctgactatgttgaggtgtgggtaaagtaggagaaaagagagcagaagatggaaaatggaggaaggagaaaaagcgagagtgaaatagaaaaggtgaaccttgtagaaagtgccaaaatgccaccagcagtcatcagaggggtgctttcttccacatgtccaatgacttatccttgagtaagtcaatgactatgacacaatgaatcaaattctgtttttcagaatgccagctcttaactctcttcatctcatttttgtttcttctcttgttattcatagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagaggtgaggccaattaaaattgcagctgatgtgaagagagttgtgactggtgcaggcaggagtgtttttccatttccacatctaagaatttgttgagtttgttgcccaaaggctgggagtttgttcaatcaagctgttaactgtcttgtgaaactgttctattcagacttttctacaaagtaattaaaaacctaggttggctgtcagagaatataattagaagtaatctttcatcattattactatggtatgaaactcgccaaaaagcaaagcaacaatttatcaagcataatgtttgattaatatagttaaattaaatccaaggaaattaatgctcactaattaaataaatacttaaggattttgtgattgttgttcatttaaaaggagatttgaatacttccacttgcagtagatactattactaaatagatttaaatcccatagtacaacattgcctctctttgcaggtcagagtgttgtaacctttttagcatccactctaatgatctcaaccattgtaaatttatacatgaagagccattcaaaaagtacctggtttggaatcatgggctgtcatttttagagcagattccaatttttatattactgtcataaactcttattgtaaacaaagtggcccaaaaccaatcacatggaaaaggatttcagctacatactagacacttacagggctatattattgaaatttacttcataaaccataagaagcttttaatgttggtattaaataaaattccattagctatcaagacatattttggcaatgtcacttgattgtattttatagcattcaaaatgtcttcttatgatttttttttcacatagctccatttattatcattgaacaagctctttgagccacattaaaatgatacggagttcgttttcagttacctaatggaggagcttcttatcttggattataaaatagccattatcttcttcacattttttgcatggcctctccccacctccttctaccagagaagtgtccaggtatcctgcagtcaggttgaaccatgagaaaagtagaactttatagtggaggaaccaggaagaatgaagagaggacatcagctcttcttaaaaaatgatcatagataaccatgtaccagacactgtccagaacactgtacatgtgttaagaatcatttaagcatcataacaaccctttttgatagttagtaacattatcccaattttacagaggaggaaactgaggcttgtgcgtggtggagctaagatttgaccccaggtatgctggttactgaacctacattctatcaacagtgaaatattgcctcccactgagttatttttaatttctttaaatcaaaaagaagagatggttaaggaaataaacacataaacactttcattttaaccaatgtttctcaaaatatacttcactttcatactacttacatcagaatctcatgggaggcttcttaaaactagagattcctgtgcctacccagaccttttcagaccaaaccactgaggagaacaaggtctgggaatcttcttccttaacaagcatcccacataattcttatacataataaagtatgtttatcactgatcttgataaatgttaattgggtaaacaaaagcaaatctacaattaccatgcaggaatacagacagactgtcagtctgtcagaaattatttagcatttatcaataattatcataaatctcctgtcctatcagagatgatgggacaaatcgctgaaggcaaagttggggccagcttgaagcaaagctttgtgtggtccctttatttcctgcttcctcaacttcattctttaatcttacaatcttaagtgctttgaggcagggcactgtactattgcaaagttgagctgaaggtgcaaacaaatgaagtaggcttttggagaatgcagaggtgaaatgacaatagaaaataaatagctatgggcaaatgacacccttgaaagcacatcatttcctgtactttacacataaattcaatcgagtaatgtcattagcagttttggaatctatttgaaaattagacaaatctaggtttgtacatgtgcttctgtgtagaacagaagggactagatgatcttcatgtgactattttttttttccttaaaactttgcctctttctgacaagctgaaatattttaaattctaagaggcaccctttggattaaaagacttttatttttagagataggttatttcttttcttactaattttacttgtttttttaccttaaaattaattttagaatgacctatatgaatagttatcaccattagtgacaatcatatgcaatgagtggaaattttggttttgaatatgtatgcattaaaataattcaacttacaaaagataaaatactgctaattgttcacatcataataggatgtgaccaaaataaataatattttgatcatattattatattttgatcatattgttcttttagaaatagggaaagtcctcaaaggaatgaaactttttaatttattattaacactcagacctgcatttgaaattctttagctttacctttttttttcctgtgataattgacatcattgtttgatctcctgaagtaggaataaatttccacccatgttgaaatcctgatgagtttattctggagtaggagattatcagatcattgtatcattactaaaaatcacagtcccccacattggtattatctccttaaattatagtctcagtgccaagggtggattgttttgtggattaagttgtttttcaaatataggacaaagttatagactagttctaaaatttagttttgtaattaggaatgttgggaaatattacctgtgtctaatgaatgaaggcattttgcaactggaattcacattttaggggaactgttactgatgcatatgaaggagactttcaaacctttttgttcatatattaaactacctgaatatatgtctataaagatctaaaaactcaacctgggtgaaaattaagaaacaatatgttttggtctgaagtcctaagtgggattggctgaaatgctaaaaggttatctgtccagtagtggacctggtccctccagcccaaatccctgggatagaggcataggaaagcccaccttgacaaacccagggctccccaaaagctgaaaatctgacagacttttaaacaacccccaaagaattatcattccaacaatatcttagtgagctttttacatctgagaaagcatggtgtatatttagttaaataacacctgttgtaggaatgctttgggctttgctgctttcaaaaatagtggttatttcatctgaaattctacttctagggcacaactactgaaacagaaataagaaagagacggtccagttcttatcatgtttccatggatttattggaagatcctacatcaaggcaaagagcaatgagtatagccagtattttgaccaacaccatggaaggtatgttaaaagtcctgcgtcacagttacttggtgctttggtaatgatgaaaaaacacttcataaatttcaataaaatacttcctgacttgatattgtatcattattacacattttactaaataacagtaaaatccgtgcataactcatggattctattatcttccacagattttttttttttatatttagcctccagaaagctgctgcaaatgtaaggtatattttgaacaccactttcatacattaaattctaaacattgaaacttgtgtgcatgacgttgaaaagagtgtaatgataaatgcttatacttatgatgatgctaagccatttggattatattaactgcttgagacacaagttataaaatcctatgacttaaccagaaatataaattaaaaatgtgaattagggtttgatattaacttccttgaagcaaagtgtttaaaattttgtagtcctacttttgcctttctctgaccagattcttacaatatatcagctttctctttagttgcagattttatctgaatagttaacataatgtgtagcagtctggatctcagaatgccaaaataaagactttggggacagcttaatctgtgatcaatttctggctctgccatatgttaaatgtgttaatttgtgactttgaatttcagtctcctcatcagtaaaatgtggatgatgatgtttaggcataaggttgttgaatggattaaataagccttcttagataaaacactgatgtatttggcatgcagaagacagttaataaatattatcaatattagttgttttgttgttgttatttttgttaattcacatgtttttgcctttccatactgtaagtgaattcaaacaactgtcaacttcaactacttggaaaatattttcatgtaaaatgtattctatcccccttccttgccctcctattccctcctctccctatctctttacaaaccttctcccttgtaccccttcccaggtatgtgtgtgagtgtgagtgtgtgtagatgtgtcaagggagaagagaaaaggagaatgaaagcaaaagagagcaagcatacacgtccctttcttattgataattagattttctcttgagattggatagattcctggaataattcttttcctgtctgtatgcaaagatcccataatattattaataccaatacgaaaagcctgaaaatcacagccagaaaaaattcacagtgtagacgactgtgtacatcacagacaagtcagtattacaaaacccaattttcatagtgtcctatttcagtatcctaatgcaattcactgatttcaattgaatattaaactctagtacgttcttccccaacctcgcctgcgttagcttgcactccctcttccccccagctgccagtagcttgctcctccctgtccctccaggtaaatcttttgaagattgtctggccttccgctccttgccatagcaaaaccactgagaggaagctgccagtggttctgctaccgatgtcagcagcatgtctgctccctaaagcaggaagtagagaaggagacagggtaagtctaaatcaacagtcatgctttgcacttctgatagcattaagtttgagctaaataagacattacttaaaaaacctcaaatatccacaagattggacttgccaactaattaagatttggagttcaaaataaatgcacccacacctttctccatggaactatgtgacatggggttgcttaggatggaaaggatgttctaggaataagtgcaatctaggaagctgaagactgagagtgttttcgttttattatctgcagagcttttgacttgtgtatttgtgagaaataatggccaagtttttattctgtttttaatcagttatctagaatgaaaactgacttttctttattcaattgtatgtagacacattgagtgtgacatttgtcaaggttggttgttagcaatatcacatacatgcatactcaagcagacttaagatagtcctttttttttttttttttttttttggtttctgataatggtgcaaagttttcctggttgacataatctcttttcttggggatcctttcttctatgtctgattattgtttatttcacctttcctttttatgaaccaggcttgttgatccggttggcaatttttgttctccttctttttaactacagccaagtctccgttgtccagggtaatggatagcctcatgcttaatgaagcagtagtggataagcaaaaatgaaccatttgcgtttcaaatttttaaaagtgcaaaatcacatagaaatgttttctggtcacacctttgtgaaggatggtgggagggtgagttagaagcgcctgaagaatcaaggtgagccagcaaaagacacagatttactgtaagtgattctatgtggataacccatgcaggaataatggagatgtggctgcagttctctcctgaatgctttgctttgttttaaagtgtgagattccccccttttttttggaatgaataattgaatgattttattttagaacttaaacaactttgctccggttattcctactgtcaagatgagccacacctgctgaatttcattttttaaacttttctccagtttattttttttctatccagttcctgtttgcttctcgttatttgctaaatgacagctggcatggaaagaaaaaaccatattatggcagtatacagacaaaattaaattttgtagtttcttttttgttatttactgtaaataataatacctcctttttaccttcaatgtaaatataaggttatttgggagtttagaagtatttacaaaataactagtgcataataactatatttttctctgaattataaatcaaatattatttattgtttacaaagttatgtattaagggaaatggaaacaaactgggcacttgaagaatttcaatatccaaagagacaattgacaaatctatttttagtggaaattttaaaaacaataagcaataaattaatttacttaggaaaatagtattatagaattaattagtggcaaatgttgattagtagaagaaacattatcatctgtggttgtgattgtccttttatatgctggttccacctttacaaggtttagttcatagcaaactgtgccagatatggacagatgttccagttgccacaatagtattaaagtgactgactagagtcaactatgccatggatttaaaaagaagaaaccttccctctatttcagtgctaagaggtggtggccacattttggcagaacaggtaatagggtgtacagcaatgatattgacagaaaacaacaaattctgcatatttttccactcataagttgatgaagagattatcttgccaaaggaagatggtagacagtttttcacttcctaattccccaaatttctttgccaatattcaccaaatttcagtattttgggtgtgaccttaaagatctgtgcatttctgtcttctctcccaatgtttggttgaaattcttcttgacatcattgtacattttccttgaataaatgcattttaatataaaattttatgtcatgtttgatatgagagttatatatttaaatacatttaaataaatgtttaccatgaaaatgtatgaattatatgtatgtttcacctaaaatcctttgtatttttccagtaataaatgagttccactttgtgaaatgttgatttgtaacaacagtgaggactccagttccttaggctggggtattttctcttcttttatgccctctagttaaatgagaaatgtagagagatggaactttgttgtgtctaatatgcaagcctataatctaataaaatttaatttgagacttttaaactgagattggtgacactgacaaaattatctaattagaagatcaccaaaacatatctaatccaagaaactgacattcagtgtgactgattaaggttcttaggacatctcctgagatatctctgataacatatatacttcttgctctacctggaacatggatgagctttaagtgtatgcaatgcaagttctacccattagtttctagcagccttgaagataagtatcagacagtttagtgttgccaatagaatcttggaagctatgtttagccaggatacatttggaaagcttactagcctttctgtactgatcctttctatgacagcaaacccattgtaaaattttccctgttcctccagcagattaacccataatatcttttaacaactttagattttttaaattccttttaatttaaaccaaatctgcttaatagaaagtaagcagttttcatgaggattctaactttttttcttccagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtaagcctcactgagagtttctcttcctcttgaaagagtttataattgccttagtgaattttacatattgctctcaaattaaatatcaactaattggccatgtatatcttgacatcaaatgtttagcatcccttttaaataacaaaaaaatgttgctaccatagtgcaaaagagtcaaagaatttatgtacaatttgatttagaattgaatttaaattgcttatttattagaagatgattctgaattgtcctccaaggacattgatctatagcaaaattctgacatatttttaagaaccttagaataggttctttaggacatgtctgtgtttactaaacaaatgataaaatatgcccaagtcagataattttgaaatatcacttgtaagtacttgaagatggactatgtagggaggggacatcatctgggggattattattttttgtttttgtttcttctacagtcttagcaatattaaatttagaaattatttatttaattttgttaaaatatatattagcatttactggatacatattgatattataatatagtatactatagtgatagattttaaagtggtcttatacagagagacaaaatgaagaaaatcacgcaaaaaagtatatttatacaagtataaaatgcttattaagttcccagcttgataaatgaacatataagcaggttatatagagattgtaaataatacggtctaaagtataatatacaatatttttaggctctgaagtaaccactatattttcgaattatatttcaggtaggacttaactgaattaaatgataaagttggcatatgttggcctcttattttgtatcagatattggactaaatggtttacattaaatctctctctctagattcccacaaaaccagtcttaagatatttactataaattgtctgtatttcgcagttgggaaactgtatccatgagaaattaacagagaaatggaaggtctgaatgctgtatcagactctgaaagctatttcagagactatcataagctatgggcaaagatcacagacgcttagagtaggaaaggataataattttacctagttcaaatttagagctatgtaagaatttcttcaacattatttctaaaaaaaagcagggtggtggggtggcaattgaaacaagaaagcctttggaggtaatatattgtgatccaaattgaatgagcttaagcaaaaataaagggaattcattacctcactttactgaagcccatttggaactagttctaggttctaacctggctagattcaggttctcagccagcatcattgggaatcagcttcttgtctctcttcttcccaatatgtgaccttcctctctggctgggcattttcctactattgccaagattctatcaacttttattctactaatgtagctccatgtacagaaagtttgtgcctcctaactagtagctttaattaaggcccacaaattgaatatcattgacctggttggagttacatggccatttctaaaccagtcagttaccctagctcttggttttaatgccatattcgcctaaatcagaatcatatcttatcactggaggcagagtaaatgagtgaaattatgaagacaataattaggataccattaccagaaggaggatggattctagaaagaaataaacaataaatagacaactcaagctggggctggtcttacgtgttaaggaatgtaggatctgtatctaaggtgaattatggaatagtagaaaatccataggcaggatacacattcttctcaaatgcctataggatattccctaggataagttataggttaggtcataaaacacacatcaataaacttaaaaaaattaaaataatacagaatatttttctgataaaaaataaaatgaaattagaaaccaataacaagtcaatgtggaaaatcacaaatatttggaaattaaacaacttgctcttaaataaccaatgagtcaataagatgtcatgagagaaattagaaaatactttaggatgactgaaaatcaaaacaaaccaaactgaaattaatgagggcagctaaaacaatatttaaagacaaatttatactctaaatgtctatagtaaaataggagatttcccaaattgctaatttaagctcctttttaataaactagataaagaatagcaaattaaacctgaagttaacagaaaagacaaatattaaagaagagaaaaacaacagagaaaaatcaataaaaccaaaagttatttctttgaaaaaatcaacacaattgacaaatctttagttgggctaaccaagaaaaaaagaggaaagacactaattattagaagtaggaatgaagagaggatattaccatggatcttttagaaagaaaaagggagcataagaaataaaataaaattaaacgccagcaaactagataacctatgtgaaattgaaaaattcctagggagtaacaagcgcctgaaactgactcaagaagtaatagactatctcaatatacttataataagtaaacattgaattcacaattaaaaaaaagaaaacttcctgccaaaaaaagcctgagcccagatagtatcactggtgaattttgccaaatgttcaatggagcgttaacatcaatccttaacaaactgttccaacacatagaagagaagggaatacttctcacctcactttctgaagctagaattaccctgatattaatgccagacaaaaataatgcaagaaaagaacacagacacaaatatagaccagccatatcccatatgaacataggcacaacaatcctcaacaaaatactagaagccaaatcacataacatatttagatttttagattatgtactctgaataagtgaaatttatcccagtgatgcagggctggaccagcataaaaaattaatgtaatatatcatattaattttaaaaaactatacaatcatctctatagatgctgtaatcacatggaaaaagccaaagtgtttcatgataaaaacactcagcaaacttgaaatagaaaagaacttcagcctgataaacaccatctcaaaagaccccacacctatcatcattcttaatagttactttagatgcttttatcttcaggtcaagaagaaggcaaggatatttgctcttgctacttcttttcaatattgtactgaaagttctaaccagggaaataagacaagaaaaagaaattaatggcatctagattggaacacaagaagtaaattctattaacgaatacataatcttgtatttagaaaatcctataaaatacacacacacacacacacagctgttagaactaataaataagattgcagaatacaagatcaatatacaaaactcaattatatttctaaacactactaatgaacaatcagaaaataaaattaggaaaattctatttataacaacatgaaaaataataaaatacttaggaataaatttaataaaataagtgtgagatttgtacactcaaagctataaaatattgttgaaagaaattaaagaactactaaataaatgaaagcacattttatattcatagattagaggaaaatattgttaagatggcaatactcaccaaattaatctacaaatttaacgctattcatatcaaaatcccagctacctattttgcagaaagtgataaattgactgtaaattttatatgaaaatgcaagagactatatgccacacaatcttaactagaaaaaaaataaagttggagaactcaaacttccaaattttaaaacttactacaaagcaaaagtaagcaagatagtttggtactggcataaggatagctatatacatcaatggaataaaattgaaattccaacagtaagtcttcatatttatgttaaattaattttcaacaagaccactagacaattttattaaggaaagaagccttttcaacaaatggtgcttggacaagtgaatatccacatatgaaagaatggaattgaacccttacttaataacatatataaaaattaagataggtcataggcctaaaggtagagctaaaactatgaactgttagaaggaaatttagaagtaaatcttcatgacctattattaagcaatgatttcttagatatgataccaaaagcacacacaatagcaataagaaaaaaaaggttcattggactttatcaaaattagaaactttcatactgcaaacaatatcatcaagtaaaaagacaacttaaagaatggaagaagacatttgcaacccagatatctgatcatgatttgtatctaatatatgtaaaggattattataactccacaacaaataaaatagataactcactaaaaatgatcaaaatatttgaatagacatattgaaaaaggagttagacaaaaggccaataagcacataaaagatggtcagcgtcactggctaattttaggggaaaggcaaatcaaaactaaaatgagataccacttcacacacactaagatggctataatcagaaagaaagccaataccattttttatcaaggatgtggaaaaattagaatggttatgctttactttgagaatataaaatgatgcagtcactttggataataatttagctgttccccaaaaagtttggggtagagttaccacatgacctggcagttttactaatttcttcggatatatatatctaagagaattaaaaacatatttcaacatgaatgcttatagaatattattcataatactaaaaattaaaaacaattcaaatgtctatcacttgatgaatggataaacaatatccatccaataaatgttattcatccataaaaaagaatgaagtattgctacaatctacaacatgaataaaccttgaatatattatattaagtgaacaaagccagtcacaaaatttacatatcatattgctacatttatatgaaatgttcataacaggcaaatccatagagacagaaaggagatgagtggttgccaggcagtagggattagggtaaatgggagcaacaactaactaatgggtatgcagcttctttttaaggcgatgaaaatgttctaaaattcaataatggtgatgattttacaagtctgtgaatatattacaagccactgaattgcatactaatatttcatagtatgcatgtatcactattttttcttccaatcacctattgatggatgttcaagttgattccagatactttacccccatgttgcagtaaatgttcttgtatatatcacctttcataatgctgtattaacttctagaagatagatttccaggattgaagtattcccaatattaaacatattcatgtcacataaaaattagatgatatgcaacgaaatagggaaatgtgactcatagttaatagattaaagcggtacatagaaatagatctggacaggaacaaattgttagaataaacaaaaaaggactttggaggcactattgtaaatatgttgagcaatttaaaggaaaaaaaggggtcataaggaataaacagagaacatgagcagaaaaaaatgaaaactgtaaaaaggaataactcagacattttataattgaaaagtacacattctaatatgaaaaattcactggatgtgtttaaaacaaaattgcagatgtcagaagaaatagttgttaacttggagcacattaatagaaattatccaatttgaacactagagaattaaaaggaaaaatgaactgagtctaactataatatgggataatatcaagtagtctaaatatttatataatgggagaatcagaaggagaaaatagaaagaatggaacagagcaaatatttgaagaaacaatggccaaaaatttcccaaagttggtgaaaaatagtaacttacagaccttaatgttaccttataaaatgttagtgaacatcaacaggataaatataaagtaagccatacttaggcacatcttagtcatgctgctgaaaacaacaataacaaaaagctttggaaacaaccggaagaaaaatatttactatatacatgagactaatgtcttaccagaaataattcaggccagaagaaagtggccagataaaataaagaaataaaagcccattattctacagaaagtgaaactatccatcagaaattaaaatgaaataaagaattttaagataaacaaaaacctaaaaaagctgttaccaccagaactacactacaagaaatgttaatggaagttctttaggccgaataggaaaaatatcagatggaaatttgtttctgggaatgatggtcactagatatgaataaatgtgggtaaatacgaaagactatctttttttccttaatttttttcacatttattttaggttcaggggtatgtagacagctttgttgcataaataaattgtaaatcacaggggtttggcatacagattatttcatcatccaggtaataagcatagtactctatagatagttcttcaattctcatcctccttccaccctccaccctcaagtgtctgttgttcccttctttgtgttcctgtgaactcaatgtttagttcccacttataagtgagaacatgcagtatttggttttctgttgctgtggtaaattttttaaaaagacaactgattgtttaaagtggggatttataacataggtaagtgtaaaattatgaaaagatttgaacttttttttttttttttgagacggagtctcgctctctcgcccaggctggagtgcagtggtgcgatctcggctcactgcaagctccgcctccccggttcacgttgttctgctgccttagcctcccgagtagctgggactacatgcaccagccaccaagcctggctaattttgttgcatttttagtagagacggggtttcatcgtgttagccaggatggtcttgatctcctgacctcatgatccgcccgtctcggcctcccaaagtgctgggattacaggcatgagccaccacgccgggccagaacattttatctaaagtgttacaatattaattttacatagaaaattacaagaaagtatccctcatgaacacagaagcaaaaaatcattagcaaaatattatcaataaaatctagcaatagagaaaaaagtaataaatacttcttaaacatgtggggattatctcagaaaagtaagattcatttaacatttgaaaagtgatcaattaattggccatattacctctaaaagaataaaggaaagcctaagatcatctcaatagatgcagaaaaggatctgacaaaactcaacagtcatttgtgagaaaaactgtcagtaaactaggaatagaaagtagctatctcaagttgttaaggacgtttcagaaaaccctacatctagccgggcgtggaggctcacgcctgtaatcccagcactttgtgaggctgaggcagtggatcacttgaggtcaggagttcgagaccagcctggccaacatagtgaaaccccgtctgtactaaaaatagaaaaaattagctgagcgtggtggcaggcgcctgcaataccagctaacagggaggctgaggcaggagaattgcttgaacccgggaggcggaggttgcagtgagccgagattaagccactgcactccacacttcagcctgggcaacaagagtgaaactctgtctcaaaaaaaaaaaaagaaaaagaaagaaaaccctacacctaatattttacttatcagtgaaatgttgaggtagtgaattcttgccccttagaaaaattgagtgctttccccttaacatagacaaatatgtctatttttaccatttttatttgaccttgcactggaagttttatcaactgaactaaaggaaaaaaaaacaaaacaaataaaatgcataaatattgaaatgtggtaaattatctatttcatcaacatgatcatattgtagacaatcctaataaatctttaaaactgattagaaataaacgcgatattaaatattttatttaccataatatcaaaaaacacgaagaatatagtaataaatttaacaaatcatttcgagacacctattactaaaaacctcaaaacatggctgagaataattacagaagatttaaacaaatggaaatatatatgccatgttcatgcaatggaagattcaatattattaagaaattaattctacccaaatgtatctatcaattcaacccaatcccagtaaaaatgtcacagattttctttgtagaaattgatgaactgactttctatgcaaatacacagtgcttaaaattgccaaaacaatactgataaaagaataatgaaaacattaacctacctgacttcaaggcttgttataaagctgtactaatcaagaatctacagtattggcataaacaaagatatcaatggaacataatgctgagttcataaatagatccaaactacatgtcaatggattttttgaaataaacactacagaaaggaaatagaagaaaggaagtgttttcaacaaaatatcaggaacacataaataaatgtatggaaaataaatgaacctccacttcgatcttatgccaagtgccagaatcaataaggattgtagactaagcagaaaagctaaatcaataaaacatctgaaataaaacacaggagaacatctttgagaccttggggtaggcaaaaatttattggaaaacagacaaaaagtgctatctattatttaaatgtccatgaaatttcaaacaatggagacctactgaacaaaaaagaaaagtctagtaatatacacatgggtgaatttcaatttgaagtattcaagtaatatttggaaaagtcactaagactgtcaggtctctgaggtaaccacctaggtagagagagattccatttactgagttagcaaatatcagaagagaagcaaaactgtggagaaaaagcagtaagatgaattttggacctgttgaatttgagatgcctgggagatagtctttcaaatgtaggtattgaataggcaggtgggtatgtatttctagagactaggaggtatgcttgaacagaaaaatagattttgaaatatgaactattataaaaatgtaacttattataaaaggaaaactaaagtaagaggggtctagacagagagagttctgaataaatccagtatcaaatgatttgttagaggaagaaaatcaggtgagtagcatccagggaggtgtggatcacagaagctaagggcagaaaatatttcaatgaacaaggaacagaaaacaatgcctgaaacttctaaaagggcaagcaagcaagataattgtttaaaaaatttcgtttggatttattggtgatgtcggtcttgttggtgaagtttgttaaagccatttggtgggttgtggagtaaggtgaagaaatggaactggcaagtgtagacaagtatgcaatcttcgaaagaaatctggccataaggaaaggatagatggtggcacctggaaggggaaatagagtgcaaggagagtttctcttatgacagtggtgatatatatattttttgtttgtttgtttgttttttgagacggagtcttgctctttcgcccaggctggagtgcagtggcgctatctcggctcactgcaagctccgactcccgggttcatgccattctcctgcctcagcctcccgagtagctaagactacaggcgcccgccaccgcgcccggctaattttttgtatttttagtagagaagaggtttcaccgtgttagccaggatagtctcgatctcctgacctcgtgatctgcccacctcggcctcccaaagtgctgggattacaggcgtgagccaccgcgcccggcccacagtggtgatatttttaagggagagaaggactcggtaactgttactttctataaagaaacgggaagtagccagtagtagaaatgttggttaattgagaatgaattccttgcaaagtccaaaggaaaacacataacacaatttgagagattagctcaaaacaggggcacttctttcattttaacaagaagagaaaggcaaagcacagttacggatgtaagtagtctagtagataaagggaaacaaagttcaggctggctgactttcatcacctctaagacgctgaagtactgagactgtgcttctcagatgggaaggcataagcaaaaatggctgaggttatgtgcagaagagaaagtttgaaatagttttagataatagaaatggagaaaggaaaatactgcttccctttcgccaacaaaaggaaatttttttaagagttcttactatctgtagagctagccgtgagcatgtttattacagcttacatgacatcttagcaccagtctcatctgcagggccaagggaggggaccaatccatttggcttggtgatggaagcccgcactgctaggtaatcatttggtaagtttttggagggctagaaagatcagagacagagccaaacagttgatcacaatgagtcagttgcacctttcatatgaaaataatattaattttattgacttaatccgtgtactctttatcatttgataaacattatatatagtgaacaattattgatttgaatgcaaagcatttgtagatactaagttgttggacctaaaccaattttttaaaatcagaatttaatttatatttgttgggagtaaattaagttgctcaataattattcgtgtttcaagagtatttgctcatataatgaactacacttctcatttaggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctggtaaattaactgggagtgttcataaaatgtactttgtaattaattagtcttcattctcatctagtaaaaatggcaagatttcccatcattataatattatttgaatacacttctaaaacaaattggattgccataccaccaaatggtagtttcttcttcatcatagctttaataaagttcacttaaatgaatagtctacacttctcttcttagttattgaatggaaggctaatagagaggaggaaacagggagtcacagataaactcgaatcacaattaaacaacaccatagtcaactctcagttatctgaggtttgcataactgcgtacaaagcttccttgggacctaggatgagctcccctttctgccaggaactaaagaattatggaattgttcattgctcaccttgtccccgtagaggaaagagttaagacaggggatagtgtacaaaggagaaggataagcaaacagagctccccatatgactgctgccacatcagaaaatcaccaaatcactctttgaaagagttaactgtactatattttgttaattttaaagaaagtatctttctttgatcttttataaaaactattagatcttaaaattcagagataaaatatcacttgacacatttccagtgaaagtttgatatgttttgttatactattactttgagttggctctaagttagtgatttattttcaaataacagaggctgtacacggttactaaggacacgttcctatagatgatttaccttagtagtgattaggctgaagactttttcatgaaatctgtttacaatttccctttctgctttcaatgttcaaatttgagttgtaatccttagaactatatttccttccctaatcctcaaagatagttatgaatctaatttgaatctagaaggatgcaaaaaacagaacaaaaatttaaaatgataaaacaagtaatatgggcaagaacttaaaaaaatatatttagtaaaccttcatgatagtgtgatgcagttaagggaaataggaagcatagtatcactagaatcttacttagtgtgtcaggctcttttgcataaattattctctggaataaattaaatactttggtgcatgtatttactcctttgggtcactttgatgccattaaataatgcactactttcagcctgacatttactgaagcatcagaaataaaatgctgctgctctttaaccataaatggtacttcagtgaactctaaagctaatacaaccaatatgtcaaacacaatgagaaagacatttacacactacactgaattaagtctatgaagatataaaggttaaaaagaagcctagcgttttacttaagtttaagtatttttgtatttgaatataatatatgtttaaaatatagcctaaagttacagcaagctaaagatatagctagattaaaacaatctaaagacaaagaaattagttcatttctgcttccactttatgtaatttaagtgttgatattattctcacctgtgcatttcagcatatttaaagtacactgaaaactatatctgctttggccttttaaaaataatgagagttcctacttctctgaaactggatctctgctaattaaccaccattaatctgaaatatcttaattccttaaggagaaacaaaagtgtatattacatatgcttatgtaggatacttgaaaatttggtgtatcttattaaactgccaatttaaaaactgtataatttaattatttcatttacagtatggaccatttcaaaatgaaaaaaagaatgctctatggtagcaagtcactgctatatttgttagtgatcatttgacaaataaataattcatcattctataattgagacagttacctgtacatttgccctgttaataaaattacagatttttcccttcctgtgtccatgtgactaacctgcacattgtgcacatgtaccctaaaacttaaagtataataataataaaataaaataaaaataaaaaataaaaaaataaaaataaaataaaattgcagatttttttagaaatgcagagcattaacactgttcttgcttttatttccagctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggtatgtagcaaaaacattttcctcattttcattaaaagataatgtaatcattaaaaagtgtgttcaactgaagaatattttgtattttttaaatcaaggccacttcctattgtctattactcatgactgtaagagccatgtatagtttagaccattgtaatccacacaaacccttaaactaccttttgaaccaaagttattctttctttcattatccttcttgctacaaggagagaaacttttctgttatttatctttcagttcttgtactagagcatggaagtgttacttagaacactcattttatttataagtactagcaataacacctgaaaacgtttcagatttggttttctacaaatttaaaaactagcaacaatctcagtttattaagagctcatggggttttcggtgcctagaaactatggtatgagcaagtaacattgtctctaaaaacattaattgtcatttctgcataaaattaaccacccctaacaccatatatatttaggatagttagctcttcttgttgcattgatcccttttaccattatgtagtgtctttctttgtctttttttaatctttgttggtttaaagtctgttttatcaaagactaggattgcaaaccctgctttttttttttctttccatttgcttggtaaatattcctccatccgttttttttgtgcctatgtgtgtctttgcatgtgagatgggtcacagcacaccgatgggtcttgactctatccaatttgccagtctgtgtcttttaattggggcatttagcccatttacatttaaggttaatattgttatgtgtgaatttgaccctgtcattatgatgctagctggctattttgctcattagctgctgcggttttttcataatgttgatggtcttaacaatttggtatgtttttgcagtggctggtactggtttttccttgccatatttagtgcttccttcaggagctcttgtaagtcaggcctggtggtggcaaaatctcttggcatttgcttgtctgtaaatgattttatttctcctttgcttatgaagcttagtttggctggatatgaaattctgggttgaaaattcttttctttaataatgtttaatattggctcccactctcttctggcttgtagggtttctgccgagagatctgctgttagtctggtgggcttccctttgtgggtaacccgacctttctctctggctgccattaacattttttccttcatttctaccttggtgtatctgacaattatgtgtcttggggttgcttttctcaaggagcttctttgtggtgttctctgtatttcctgaatttgaatgttggcctgtcttgctaggttggggaagttctcctggttatcctgaagagtgttttccaacttggttccattctcccagtcactttcaggtacaccaatcaaacttagggttggtcttttcacatagtcccatgtttcttggagactttgttcgttccttttcattcttttttctctaatcttatcttcatgctttacaaatttaactcaacatggattaaagacttaaatgtaagacctaaaaccataaaaaccttagaagaaaacctaggcaataccattcaggacattggcatgggcaaagacttcatgactgaaacaccaaaagcaatggtaacaaaagccaaaattgacaaatgggatctaattaaactaaagagcttctgcacagcaaaagaaactatcatcagagtgaacaggcaacctacagaatgggagaaaatttttgcaatctatccatctaatatccagaatctacaaaaaacttaaacaaatttacaagaaaaacacaaccctatcaaaaagtgggtgaaggatatgaacagacacttctcaaaagaagacatttatgtggccaacaaacatatgaataaaagctcatcatcactggtcattagagaaatgcaaatcaaaaccacaatgagataccacttcacgccagttagaatggcgatcattaaaaagtcaggaaaccacagatgctggagaggatgtggagaaataggaatgcttttacattgttggtgggagtgtaaactagttcaaccattgtggaaaacagtgtggcaattcctcaaggatctagaaccagaaataccatttgacccaggaatcccataactgggtatatacccaaagggttataaatcattctgctataaagatgcatgcacacgtatgcttattgcagcactattcataatagcaaagatttggaaccaacccaaatgcccatcaatgatagactggataaagaaaatgtggcacatatacaccatgaaatactatgcagccataaaaaagagtgagttcatgtcctttgcagggacatggatgaagctggaaaccattattctcggcaaactaacacaggaacagaaaaccaaacactatatgttctcactcataagtgggagttgaacaatgagaacacatggacacagggaggggaacatcacacactgaggcctgtcgaggggtggggggctaggggagggagagcattaggagaaataactaatgtagattacgggttaatggatgcagcaaaccaccatggcaagtgtatatgtatgtaacaaatctgcatgttctacacatgtatcccaaaacttagagtataataataatttaaaaaaattaaccatacccaacactagtgtcctgaatcttgaaggcatggagaagttgggaaggcatgggaagataaatataacaaagtgatataacatgtactcaaatagaattaaaaataggaagtaactaatatgtgtccaaaaatatgaaaacaaagtgccatgtgtcaagtttacaaaatgtaaaccttgctttacaataggaaggttgatcagggaagtctttgtcaaagagtttggacctaaaatatatttaactgagatgtaagatttagcttggtaggaagaaagaccatcccaaacaaggaaacaaggtacccagtgactgagggatacaggacagtagactctgtgagaagtatcaggctcttatgctttaaatatgaagtaattacaccgagttgcttaattagaacccaaaccaatggaatagaaaaatgactaccataacaagtaatttaatgtatatactcttgccaggctcagtggctcacgcctgtaatcgcagcattttgggagactgaagtgggcgtttcacttgaggacagtagttcgcgaccagcctagtcaacatggcaaaaccccatctctactagaaatacaaaaattagccaggcgtgatgatgcacacctgtaatcccagctacttgggaagctgaggcacgagaattgcttgagcctgggaggcagaggtggcagtgacccgagattgtgccattgcattccagcctgggtgaaagagcgagactctgttgcaaaaaaaaaaaaaaaaaagcatatactctttagacatgatttcctctcatataaaggtaacctccaagtccccaaagatagagaaaggggaagggaaaaaggcaaagtattattttatttttattcattgccaaatttcagcctcttcaacattacttttgataattctgatctatttttaaagtaacaagaaacataaacagtgtacaatctagaattataaacagtggcttaaaacaataaacactgattacttcatagtttctgtgggtcaggatttggggaataagttagctgggtggttctggtttaggatcagtcatgaagttgctgtcgagatgttagctgaggttacagttatcttgactggggctggaggatcagcttctaagaaggctcaatctcatgattattggaaggaggtttcagttcctttttggcagttagttgaaggtctcagtttttctctgcaggaccttttccataggactgttgagtgtccttatgatatggcagctggcttcttccagggaaggtgatgtaagagagaaggcaaggagaaaatcctctttatgttctactcttgaaagtcactcttcaccacttctgccatattgtattcattagaagctagtcactaagaagagctcaagctactataatccccaagacaactttaaaatgtttgctttcagaaaagtataagatcacatagaacagaaagtgccatagggttacatagaacagaaacaaagaaaagataatataattatgttatagatttgatttcattttctctgtatgtatatttggtatatgttggaagaagaaaagaaaacgcagagaacagaatcctttatgacaacatgaatgatcagacagcaatggggaattaagaaatataagtttgggaccagattgggtagaatttaatttatgaaaaggctgactgtgcataataaaatgtattttcctttaggcaattcaaagctacagatgattttttatcaggaaagtgacagtgaaccagtgatatttttcagaaatatacgtagcaggagaatgcagaatagatttaaagaggatgaaactcagcccaccacatgttatctattagtttactgaaattaacatatctctctaatgtataaatgtgcagaaaattgaagttgaaaagagaatttcaggaaatatcaagtacttatggttgacatcagtattaatttagattgtgatgtatgcataaaaagatatagtttataaaataatcatttccatctactgggtgtaaatttaatttttgttcttttaagagagaaaaattaaaggttctcctttctttttgactatcagttaaaataacttctttgtcttgtgataacctgggtatgtttctggagtagctaaggtagtcatatatatcatgtttaccactattaaggaaatgtgcttatataacatttgcttaagactgaatgaacttgatatactcactccttactacaattcttccttcctattctcactggaaaaatgggaaaggtgtcccaaagacaaaatggcataacttccttttaacacacatgaactatcagatgtggctccacccaaatagatgtagtagtcacaatggatgggactgccagcctagtctacagacaagacagagctgggaccacaaactactgtttcccagaccaggatttttatgagccattcttagtttccagacacgatggcaagagacccttcattggttgaagataggtgctgcagaaaaagaatgtgactttctgaaaactgatagttctagaagcagagaagacaacttcctctctccctaagtgaaggtgaggcaatagcacacaggagggatgtgaaggttttggcttcctctcacaagttgggaatcaggatggagaaacaattaaaatatgtaatatgtttcaaccttgaattcaaaatggaaattatggtaacatttccattccaagaggctaatttgagacacaagaaagagttgatttcatttactgagctagcacatttgtgaaacaggattcaggatttcagtccctgagtgagcttgctgaactgttttctttcttttttttttttttttttttttttttgagacggagtctcgctctgtcgcccaggctggagtgcagtggcgggatctcggctcactgcaagctccgcctcccgggttcacgccattctcctgcctcagcctcccaagtagctgagactacaggcgcccgccactacacccggccaattttttgtatttttagtagagacggggtttcaccgttttagccgggatggtctcgatctcctgacctcgtgatccgcccgaggcctcccaaagtgctgggattacaggcgtgagccaccgcgcccggcctgaactgttttcttaaattgtcatggatcacaccaaacacctgtgccagctgttatgcgcatacccttcggtaacaaaggaagtccagaaaaagagaataacttgactcacacaaatatttctagggaaataaggtaaataaaaagatagtgttgtggaggataagttggataatagtaagtgataacagctaaactttctcaaaggttcactatgtgccaagaactgtgctgaaagccacatgaattctgtcactgaatcctttcaacaaccttgtaagctgggcactagaaaaacgattacatttattttatagatgaggaaactgaggctcagattggttatgctacttagtaggtaacagaaaatcgattcttacctagcactcgaattctaaaatatgtgctcctctatgtcaagtaatctatagaactaagataaacatgctgatgaaagttagtgtctagtgggtattaataaacgcggtttcaaaactgtgtcaccacgggtagattggctgctttaaaaaaaataaaaacttcaatggatttatgagaaagaaaagtcatatgttccagggatatttatttattttcctgacagtggaatagcattgaattgagttatcaattcataaaagatcagagaaacaattcgaaaattaatagtaaaccctattatactgactaaatatggtagcagttcaaagaaaggaagtatcggtaagagtaaacatggaatactgtttcctcacttattctgcaaacatcacaattaggagaaaagaccttgtagttagactttcaaaaaaaaaaaagcttctctacttattaactgtggtcttggacaagtcatgtaagttgtgcagaagcattttcatctgtaaaatagtaataattcctgccttatagagttgtgagaaataaatcacataaaccttgaaaatgctttgcacaataactggtatttactaagagctcaactaaaaagctggttttacttttattgttattatgatctggtattgatactgctctaggacttggctctaaggcatgtttctgaccaaaagatctcccaatctatcagtaaagctgtgcttgttttttttttttttttaaagaatccaatataatgtgatagggatgtggatgagaaatttaacaggacagaatgagaaatgggagcaggctatataaatgtcacagtaaatgacatttgtaaatagagtatttgtccagtacagaggcaatataattggctcccgccctggggaaggattgatggatgtgtatcaagaaaaatttccaaatagacaaatgacagaactttaatatactttaggaaaataagtctaggaaatagcaccaaaatagataaaataaaaattttacatgcaattttttcttcctttgtctgtttttttaatccaaataataagttcaaaagcaaattacaataaaacataattttattggtaaattccagaggcaaaggagcaggtctggtcttaatgtgattatcaggagtcatagtatagagactgacagattgtcagtacactctcaaaatcaaacgtggtcttcattggatcttacatatttttactttaaaaaaaatcaccattggttagactaacttacaactaattagacaaaggtgctgtaagcctcattagcatgatagaagcatgagaatatagcaagaatgtagaatcctttttattgaagttttacttaaaaattttcctaagattctacttttgtactacagtttgagcatccctaatctgaaaatttgaaatccaaaatgctcggaagttcaaagcttttgagcaccaacctgatactacaagaggaaaattcacatctggcctcctgtaatgaatcgcagttaaaccacagtcaaaatgttgtttcctgaactaaattattaaaaatattgtataatattaccttcaggctatgtgtatgtgtatgaaacttaaatgaattttatgtttacacatgtgtctcattctcaagacttgtcattatgtacatgcaaatattttaaaatctaaaatccaaaacacttctggccccaagcatttcaaataagggatatgcaacttgtatttactttgtgcatttgcccccctttactgctatatctttcttttgttctgtatgttatgtgtgcttaaataatcaggaattcattgatattgtcaatcaaatcctgaaaaaaaattatatgactcagtcttgtacccctgagaatgtctgatttcttcgtaagttgtctttttttttttttccacaatagtgagtttaatgtcatgaatcttttcactcattcatactggtggagcctatttttaaagacccaatttgcagactgattactgtccttattcatggcaatacttcaactccacaatctttaattcaacaataacatcataattattgtataataaccattttatagtatttctcactattgtataattatagtagccataattgtcttaataaaaattgggacttttcatccagcaataaatacgtttttgtctgatttgtccagttatctaggtacaaaaaatggtacaaaggcacaaaaataaaatcatatttaaatatattgggataattgttgattttaggaataaattatcagtgtttccggaaatccaaattacatagtcaaaatagcatctgtattaggccattcttgcattgttacagataaatacctgagactgggtaatttataaagaaaagagatttaattagctcatggttctgcagtgagcttggtgctggcatctgcttggcttctggtgaagtctcaaagagctttcaatcatggcagaaggcaaagtggagcaagcatttcacatgatgaaagcaggaacaagcaagagagaatgtgggcaggaggcactacaaacttttaaataacaacatctcatgagaactcactaacatgaggacagcaccaaggcatgagggatctgaccccatcatccaaacacctaccaccaggccccacgtctagtgctggggattacaattccacatgagacttgggagggggcaaatgtccaaaatatatcagcatcccaaataaaagggttttttttgtacagttgtctatatttatcttttggaactgagcttaatagaaatgtttcatttagcaatgatttcagtattttctgcaatgactaaaaagcaaatagtgataatagtattattttatattgaccaagcatttttatttcattcactttttttcagaatagtgtatcatgaattagcagaaatgcatgttagaataaaataaggtgtcaagaacaatcttagaaaactaatgatggaaagcaattgaagcaatagaatgttttgatcacctgtttttcctgctgtgtttcaggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagaggtaaaaatgtttaaataaggagatattttggtgttatataattctgttgtttaaaattatcaggtgtttttaaattgcgtgtttccttcctgttaagaaaatagaaaatatctgtctagcaatatattttccatggaaaagttggtaataaataaattaatgatagattaaaatatagctagattaacaatatgctgacttatgtttccaatactgacattttgaattcttgacagtattcttgatatgaattttttcagtatttataaataattttaaatttctcaaaatgcctcaatttctccactttcttccttgtaatttgcccacaacagtgttttttgtacgtactggaaaaatatctgatgagagggtagttgcaattctcatcttgctatgttcttagttcttaattcttacgaaatacgtcataaaatagtattgtattttgtttgcacagacatatttactcaaggaagatctgattgggatcttggcttgatatttatgtatagtttatctttcctgaagtcagtcagtttttttgaagagaaggtattgatgaggaatcacactaaaaacatatttaaccctactgagctcagtgttcactgtttaaagaaacaaaaatccttaatacattatagaatgtaaaattctgaatttaccaactcagtaagtcctggtaacttaatgtattctttgatttacaagaagggtatgagcaacagaatatattttttgttttgtttgctattaacctgttgctcaataagtacagagttggaggtaaagagaggaatttaaaaccttgatatttaattgtttatacaaaaatgaagacaagatttccagtaattaaagtttgcactaactaacaaaaataacaaggaaaaacaaagattcgttccttcctcatacgaactgtttggcgaggaagataaaagcttctattcctgatgtcgggaaagaaagaatgacgacatgggggagtgtgggcactgaaaggtaaaatttaagtagcacaacatgatcatgataattaacaatcagccaaaattatgagggaaaatatagttataaaaaaagaacaaagatgggtggatcacgaggtcaggagttcgagaccagcgtggccaacatggtgaaaccctgtctctactagagattcaaaaaaattagccaggcgtggtggtgcgtgcctgtaatcccagctactcgggaggctgaggcaagagaatcgcttgaacccaggaggcagagattgcagtgagccgagatcaccccattgcactccagcctgggcaacaggatgaaactctgtctcaaaaaaaaaaaaaaagaactagctatttcagacactttttctgtatttatttgataaaattactaaagagtatgttattttccattttttcttgtttgtaagttacgtagtattgctgttagtgattaggtagaagtagatgtttaatgggaaattcagacaatctttgaatataggaaggtataaataacagggacataggtatcagtttcacaagaaataactgatgagattcaagggaaaagtaataaaaccttctgtcctggggcaaagaattactttaattggttgaacttaaatttttactaactagattattgtttgaaagttgaataatatcttaaaatcttattaacaaaattttgaacaagtgttgttacaatagttgggttatgctggaagggtggagtggcccaatttcatatacagtgtactgctcttatagaagctgaagtcggcatttataaaatagaattcggtcatttgaattttgatgtatattcccctctcattattttgaaattatgcctaatggtgaatatttccctaatagtaaaaaaagtcaatttttattttcacacatgtttagttttaggctgtcatataaactaagaatgaattatacagtatcaaacgttgaagccattggctagtttaatcttttagctaagtttcagtatcttttgaggaatgtttaacttgacatccagtcttcttaactttaagagattttacagccgtgggttttccaaaagagcgtgtattttgccttaacttaagccattatgtctgaagtaagagggaagtccagtgatgtggggtttagagtagggacatctcttgtttctcttgttatcattaagctttttgatttgttttcccattaagttagctctgagttaaatactctaaaataatatttgtgaattcagtatttcagaattggaggaagagaactgacctgccaggtggaagcagacaggattattttattgcttgagttgtggagtccttccaataccttcccagcatagagactgttacttcagtgttaacattatttggaggggtttttaattctggctttatatcaaactttctagacataaatttataaaataataaatgatgagggttatcgccgtgaaagaggttatgtgtaggttttgatctttcagaattttacctggtagctctacactaaaaaactagagaattaaaacaattattgaagaatttcagacactcgcatttgaaatagcatttcttgcctgccttctagtcatttttgtctggtcatttttctaactgggggacaggattacattgttaaatatcacaaagtagtaagaaacatcatgaggcttattaccaatcctttctaaattaattttttaattaaagaaaaaatgaggcttttttactggaatgtctaaatgaatttttttataaggcagactgagtggactcagaggttttttaggtgttcacagtaagtcctctgcaatgtctttgctaaatttgtatgattcttcagtagttttctgtagattctctagagtaggccatttaaaatcatgtcataatcccctatgctttaattttaatgttatttcgattatattaatgtaattccttttgtgattttgaatgattgttttttcttttagagtatttaataatgtggaagccatgcttgaatgactatttttcgaagtgaaatttagtagtgcgatatggtgaccttcaccgcttaccattcttacttctcacaggagtaaaatcaagctggagccatcaagaatgcagctctggtgttttttaaccagccagaggctcgtgccaccacttttacccaggttacccaagcaagttgtacatctataaatataatcagtttctaaatgacttttgactggcctgcatgttactcagctacgttccttgcccttccattggcagtaaaataaaaacatgcacagctgctattatgctgagtcatacaaagcatggtcaggcaagtctgacaaccctaacttaaaaaaaagtgatttagctgctaattttcttacatagattttaatagaaattttattcaatgaaaagtaaaagtgcatgcctttatggattatttaatttccttttaatgttacagagttttgaacatattaggagcccaaaggagaaatgtaggtgctctttgaaaacttgcaaaaatgctttttatcctctgtctttaaaaaaaagatagcccagttactgtacttaagtcttgacagttttttatttagtgtaatgtttttctgaagggtaatcttcaaattaaagcaatcccttattcatatgcaaacttcccaaaggatgttttaatgtgataataatgtaaatgaataggaatgtctgtgtttcagttgctagcagcatgggtataatatttatctgcttcattttagggaaaatggcactgctttatttaggagttgaccaacagtattttgtatttagaatataatttctttggaaagtctgtttatatttacccttaaaactcttagactgaaagaaaaggaaatcatgtcttttgtataccaaataataataataatagtgataatgagagacttatagatggtatgctccttctaaaaatagatttagagtccatcttcttcattttcttggctcctctgtgctttctctcccctctattttattgagacctgctggaaaactttctcccgaagaatattatttaaatttatcatgatccacaaactcctgtataggaaaagaatcagaaactcttgctcctagggtgtttttaaaatgaagagacttccctatcatgtgacaatagcaataaacgtaacatcattctatgggatccattagtcgaccttcatttcttaatgttgaaatcacagttttatgcacaaatatttaaccaaaatgcctaaacccaatttaatcatttttaagaaatgttaattattttgtcacttagatacagtttcctctccttttgccaataaaactataaaacagcactaatataaaagtgtagttggctatttggaagaagcaataatcatgccattcctggagcattcttttatactttggaacaaaatattccatcactggctctccagattcatgagctataatgcctcatatattggaggaatgggatgtaaaatgggatccaagatgcgtaattgtttacagttaaacacagatgcgcatatacacagggactacagataattactttttcctattatgtattaattcttcagaaaagcatgagatttaggcactttcggataatagcttgtttctcggaaagaggcaagggtagtttccttattctctgagtatcccattttgccaatttcctgtttagaaagatacttgaggcatattatccatcaacgtatctaggggattcagctggagtaaaggtggtagaatagaagctaagaaggaactggttcgtttattttcaatcctcacattatggcaatttttgatttccttgtaaaagtctatgattctccctcaggaaacattgtccacttcctaaaaaaatatactaatttctaatacaggggtttggaaaggggacaaaaatgtgcagggaaggtttgcgtaagcaatggtggaatgggttcaacagacacctgtctatgactttatcctggagaatgtgtagtcctcatgggaaagttttccagtgggatagtgattaagatggaaaaaaatgcccaaaatatctttaatataagaacaaaatgggccaaacacgtgtctttgggtcactggtaatctactgagcagtaggacatcatgacataagagttccttttgccatccgaagaaaaatatttaaaatcctattatttgtggttttaaaaatgttataatgtattcattataaacactaaaatgactttctggataatatagtatactgtgagtaattattttgattttaccatattcttttttagttctcagaaaccaaaattgtcagatatgggatacttgatttaatctgtatttgaagttttctcttttttaagtgccaattttttaattaaattaaattaaaatctctctctcttttcccaaattatatacaatatctactaattatgttttcttcgaaatgtatcttagcttcataatgagaagtgagtgtgcccatgaaaaatttaataggaagttatgttttctcttccattttctgttgtgattcattatttttgaaaataatttactttcatttgctcacatttgctgtctaaagaaaaactattcatctggcacattcatatttagtagtattattaaagcagaaagcataagttggaagtataatatctaaaaatacaaaatgaagtattgtaccttgatgtttattagatcattaagcaaaatatgattctgccctgcttaaatcatttgattataattatccagcatataaaagaatcacagtagattttcaataggaagagtcctataatattaggtatccaccaaaaacattgttcaagtaatatttccacctgaaagtaaatgattgccaatgctttttttcagagcatataaaattggctattcctatttgatctcgttattgtcctggcatccttattttgttaaattttaactaggcaagggctatgctacaaacatcagttagtccgctagtttcctgataaataagtacaggtaattaaaaagtgaacctaaatatccaaattataccaaagggacatatatagaactttttaaactggatctgcttctagccagttcatattttggtcacttactaatgtagtatttcaccataaattatgcctagattggagcatttacaggcactctttatctgaaaattcttaagtgcatgagttgtaacagtttcacatagtgatcatctcactgttctaattggtgactatgtactacagttaggttgatcgtaattatgaccttaaatgaagctgaatatttttatattcctaatttgattaattttattttatgggcttttacacattttaactgctttagtcaacatattttataatattatgacatttgcctagaatgtaattttaagaaagtcatttaattgatgttatcaagaagggttttataaatcagaatttcctgcctatgtttcaagatcattgcttaaagaaaactttttgttcatgtaatattccaatgtgtatttagctttactctacattaaaatatgttaacaactatgaatataacttgaattaatactaaagttcatggttttgaaacatggaaatcaacaatatcataagcactatcttgaacctacaatatttgattacatatctagtctactaaatgttttaaattgataaacattgggtttacttttgaatcatcaaaaagattctttagagaagcttggcagaatggggtgcagaagatctggaatccaaccactgattcgctataacttttcacaagacgataaaactcacatttccttctctaaataaatactggattggctgacattaaggatcaatgtgctgcctagattctttgttatttgtaaatcaagtaccacaagtggaaaagtattcaagtaacacatgtgacagatcctgtgctgctccgcttcagaagacagtggggaaggataagattgcattccttaaagaggccccattcatcactggagctatagatccttgtatacagagtgaaaagagggaaaacactgtcaaaatgatttagtaatagttttcctgactccacagttaaactacagttcacctcatacactcacattaggtccgaataattggcagactggttttaagacaatactcctagttctaagagttgttcgtcattgcccacacaattcagaatcttaaaagatttgtgttactctgcaattaagagaaaatattgtgtgtattcttttgaatgtgaaagtaaatatcagataggaagtgttagtagttagtggttggataaaagagtcctcgcactggtcattcattcatttacccaaaatttattaaacacaaacattggtgctagagatacaagggtgacgaaaacatgatctttcctccaaaaaattaagtctgatgagatgcattttctagaaacacaaaatacttttgaactgaaccttggaaaaagtaaaaacttgacctttcaatagataaatatttggctttaggaaaaaggtatcttaattctacatcagaactaaggtagtgcactaaaatgaaagggagcaatgttaattcttctacttttaatgtgatttaaataagagaaaatacaggaatgtcttttataatttgaaattccagagaaaatgaataaaaaagcaattaaaaaaaacacctcaacatgcttctcattttcagccaagtacagcaaactctgtttgatattctctgattttaaccttggatcaaactatttggcaaattgctaatttgaacaggctattgaaaacagacagtgtatctagcaattcattcattcattcaatacttaatgaacagcattttggcaattcaaagcctgttccttgtattgaactatattggtgtattatccatatggcctagagtacatgtgtattattcatattatccatattggtgtattatccatagggcctagagtacgtggcttctgcccccaaatgtcttacaagcatgttaaagtacccaaaatccatccaactgaaataatttgacccatacattatcaaggttcaagatgtgtgataagccatgaaaaggttggtgaatagcctagggtgcatatggtgagtgaagatggatggaagaaattaggagtccagatttattggggagactttatgaaagcaaggaatcatgattgttgcaaaatagacagtgaagaaagaaatggtgtggtggtgaaagtcttcccaaagtcatttaaagtattggcaacccagatacttggcagcaggagtgataattgatgttacaggaatcctatgtaaacttttgcacattttaatgaagattttctataatatgctgtctggtgacttctcccaatcactgtcaagggctagacttcattattttaagatgtctttcattcatttattcactcactcattcttttttttaagcaaaatttattaagacctgtaacatacttagcactgatgtaagctctgatacagtcattcagtaatgtcagcctgagcatttgcagaaccctgaatatggatcctcatttttcccttggggtcacccgaagtcttaccccgttctgtgtttacagtgtgaaccttattcttataaggaatattttttgtttcttattgtggattgtctgtacttccatcagatgtgactcagcttcagtttttctgattacctgtgttattttccaacatgttgcaagtgataagatcagtattatcaggtccctaatagcataacaggttcgtgtgaattagatatattaaaatgagaagatttttaagtcattttttagtcaattacgtgtatagaaacatagttatacttatctcagaaagattccaattcaaaggataggaaattagaaaaacatgtaatgtttcttctgagaacaaatatattcaaattttaatgacaaagatattaggattatttttctagtaatttgatatacctgataaattaaaataagaaatcatcaaccttcaagcaccttataatatataatctttgatttaacttttttggacaaattaaaaaataattctctaactcaaagcaggcagaagtatctaatatttatgcttccatcattagtatttttaaagtacaagagacaaaagcatttttaatctcatgtatatcatacatcaatattttttaagtcatcagcatcactatgctaaggaaatattttatataaagaaaattattttcataaaataaagaaaccatctttctagagaaaatccacataatcctatgcccaaatataaatataacatttacagtactgactacagcatccatcttattctagtttaacatacttcagtgtgcattttatcatgtgcttacctcatcctttttataaagtatctcttatcagctcaaccatttcctaatgttatgcactaatggtaataacattgtaacaggtccgagaatgctgtggcccaaactactctgattcctgtgtggcactcacacctggccctgcatcatttctagagcattttaagaccaggttaaagactgggatcccttcatgtatgctgatcccaagaatggaccgacctggctaaagtttattgcatttcaagatataaaacaacttctattatatttttcttttggtgatatttgatttaacctaaaggaaaacaaaagaacaactaaatattcatttctgcctgtactaacagggcaggtaagagtgccagagtaacaagtagtttccaaatgcacaatgaaacacaggagtgcattggccaaagagaatgcaaaatatcagctcttgctatatagctaacaatgtgctgctctttttggaattagaaaattatagaatatatttaataacaatctggtatatgttttcatgtcaatgaaaagtggataaatttagatggttgctgtttatgtgcatttgatcaaatcttttctgaatttgacatgaaaatacacttgtgcagctttcattggttgggtcacaattttagaataaaacaaactatttgaaaaccatttgcaaactaatgtacaaaagcaagatcgcagatgattatatgactctggcagcttacataagctttctgcaggattttctttcagaatctctatacataggctcaaacagaagttatttccgttgttagcaccatattttaaagaaaaaaaatactatggtgttgtatctaatcttgtgacccctgacctttaccaaagcggattggcattatgtttaagttcttaaattacagatcaagaaaatgcatacagaagatggggggggggcacacctaattaatttttatatttagattaaagaaaataattaaatgtgtttttttgtgggattgattttcagaagctaaatgcaactagttcatctgaaggcagcacggttgatattggagctcccgccgagggagaacagcctgaggttgaacctgaggaatcccttgaacctgaagcctgttttacagaaggtaagcaaaacaataacatatgtggtcttgagtatcctcttttctacccattttttcctatttatttaaatgtctgtttatttgtctaccatctattatctatctatctgtatctatctatctatctatctatctagtaatcatctatacctatccaacaactgtacatttatttgtttttttttgcatttgctgtttgaaaaaaaatgcaactttttaaaaggcaaagtttaatttatgtaattagatattttcatttttatgaatcatttttaactctaagaaattattaactggcttttctgtggccttctaaaatatcttacaggagagaaagccaaatcacacacatctctctttagtttaaaaattcaataaataagaaagtgagagaagtaatttattatgtactattttgtgatattataatgggtaataattgataagtgtacatttaaatttgtccttgactgaaacagctcctatttcagtcaaggtcaaatattttttattatttctgaaaaaagatagatcataaaaatgccaaaatatactatgagtcatatgatatggggcaatatgtcactggagtaatcgcaaaaggattttctgaagaaagctaaaattatgtaatttgaggtatggatcagttatatattgtaatagcaatgctgtgtatcaaaccaccaaaaaccctgggctctaagctgcttttctagttttgactcctatttccttctgtgtaactcacagactttcttgtcactaagttttacttgtatcattgtttctctattcttacagcttcattttctacatatgtctcttatatatccttcaagatctagtctcaaatccatttcctccataaagctcagaaattaaagtttaccagaaaactctcataatactttgttttgtgataattgttgctttccataactatagaattgtagacaaattgccccaacttaaaatgtacattctttgaggacaaggctatgttttacatgttatagtattacaatttgttctatgcaattttttgacaatagtagatactcaataagtatttgttgaagagcctttgatctagcaatccagaaattatacaaaggtgtttattggattgttattgataatggccagatttaaagcaaacgaagtattcaataatggtggaattgggctgggcacagtggctcacacctgtaatcccagcactttgggaggccgaggcaggcggatcacttgaggtcaggagttcaagaccagccaggcaaacatggtaagaccccatctctaccgggcgtagtggtatgtgcctgtaataccagctacttgggaggctgaggcaggagaatcgcttgaacctgggagacagaggctgcagtgcatgagccgaggtcaggccactgcactccagcccggacaatagagtgagactccctctcaaaaaaaaaaaaaaaaggtggaatagttatattaattatagtaatcatatttagagaaatattatgaaatctttcacaaatttatttacttataataaagatgggaaatagttataccattaagtgaactaatcagaattcaaatatgtaaagtgtccatatagagtggaattacactcataggataaggacaggatggaaataccaacttttggtaagtttattttctttttggttcttctattttttatatattgtgtttttgtaatgtaatccattatagtagtgctataaacataaaaataaatatttattaaacaaatgattaaaaagccatatagatgattttaagatagcttttgtaagcggaagctatcttaaaaattaatgttatttacaatgtattatcaggtaataatgtaaatgaatctcccaccaacacaaatatacctaatcaaagagtaattttttgtcttcatttttttcccacatattttagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggtaggtgatgcatgatccactccttcacctttcatctgaaatcttttccctttcccttcaatcaactcatattacccacttttaaattaaggtgtttgtaagaatgagaagaaatatgtgtgacgtgtttagcacatatgagaggcttagtaaatagcaatttttgtcactctgtctggagtagccctcgggtggaaccaaactcagatcattatggtttcttataatgtttaaagaaggatctttctgactttcagtcatcagaggcagttcttattaagactggttatgtagacatgatgtaggattatcagctaaatatcagactgaagcacgatatttccctgacccctttgcaggtgagaactagagtgcatgggtgccggtaggagcgaactccactcactcactgctccacccctcacaggagggggagcgcaggtgactgggtgcaggagccaaggcaaatgcatttgggcactgcaagagtgaactccataccggccccacaggagcgtctaggggagggtgcctgcgatccttgaagccctagaggaagtgttacagtgcccttttagctttgccatccatggatggcttaaatgttaacagttcagtggagggtcagagtgacagccttttgcacccacacttgtggtacccaagttcatgtccggcgtccaggaggaatgagtttgtacaaatgacttgaagatggtaaatacaggggattttattgccagcgaaagtggctctcagagggaagaggagctgaaaggagatggagcaggaaggtaatcttcccctggagtctggccatccccagccagactcctctccgaagctatgctgtcaagctgtccctctgatgtcaagctacttctctctaatgtccaactgtagtctctgatgtccagctgttcctcctgtctgcctgctgagttctgggctttatataggcacaggatgggggcagggtgcaccatgggtggttttggaaaaggcaacatttaagtgagaaaacagggatgtatattctcactttgggccacggttccaggcttgagggtggagccctcgccaggtacccgtcctcttctgcccagaatttctctgcctcttgttcctgtcaaaattgcttaacataaactccatgctgcaggggactcctctgtcttcttcacactgattcgctattgccaaccacagtgaatgataagaagtagactcacttaattactgactagcaaaaaaatgatggcattacaaacttatgtctgatttcattcaatgaaatgatcaactggatcaaaatattaatataatgaaaatgatatgacctattttcttaattggtgatacaaatgtggttgcattccttttactgtttcaatttaattaataactagagtgtttggtgagttgatttcattaggagaattactgcattggatctggaggcctctaaggcgaattctgatttgactaagaatcctgtgtcctgccatatactcagtttaaagaggatcagccatgctttattttctttacctttattattattattattattttttagacagtcttgctgttgcccaggctggagtgcagtggtgtgatctcgactcactgcagcctccatctcttgggttcatgccattcttgtgccttaacctcgcaagtagctgggattacaggtgtgagctaccacacctggctaatttttgtacttttagtaatagagactgggttttgccatcttggccaggctggtctcgaactcctggcctcaagagatctgcccgtcttggcctcttaaagtgctggaacgacgggcgtgaaccaccgcaccttgccagacatgctttctaaagccaagtagagagagaactatgaagtctcattagtgactagtacctttgctgtaggagctctttgttctcagttacacccagtcagtgctcaccaaattgcacaacgtgctggcacagtggctggctcctcaggggtttacagcttcagctataagcaaagcccagaaacctttaggtccttgtatggagctctggttacaagccctgattcttgttatctaaaaaagaaaatgttcctttgtctttaatccaggctgccaggttttcctgataatttttccgataagaagatcaagttagataaatagtcttttcattctggaagcctcaggagttcctgcaaatgagttacccactctttcccaagggctctggaaaattctgtcaaagggaatttccaaacgtacacccacccgcctccacacacacacagacacacagagagagggagagagacaagaaagtgagcaatgacaatcctttcctttttctgtaggctgagggacctccctgctttatatctgcattactagaggatgcattccattgagtctgcactgaatgagaccaatctactcccaggcgttccactgcctcctgatgtagagagaagcagctggcagtctctcaaaaattttaagctctttgggggtacactgagaccaaaatttaaaaattactgaaacccttggttgactgaaatgcccagtcagcagtcatttatgatcagataatgataaagtaaaattcagccatgggaaacattaaaccttccagccttaggcacctgataagagcttgcatcgtttccttttttaagaaatcatcaattagagactgtttctgatcataaaatttaatagaattttttgacttacaggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtgagtatgctgcactttgctgctttattcattggcatatatgtaatagttctagcaatggtgcctgacacagtgtaggcactcagtaacactgtatcagcccaaatataaattatgtttctcatttcacagtgagaggatgcctcaaaacattttttaccaatttaaatacatatacattcatagataaaaatcaaatgccatcatactatacttattcacttaatttcaaattaatatttaaaatctcaagttatgcaaaataaaatatgaatttagaaattttgctttttgcacactcacatttcgcaaaataacttgtatttaaatttttcacaggcatctttgacattagtatgtttgtcatcactaaagcctgttgagtttaggtcacacagatgaatcattaattacaaagaaatttgaaagtccaaaaagcaagagacaccacttgatttgtatgatatagaagcaaattggctattgaccaagtagccaaagattttattaaaccacattggtgttgaaataaaataagatagagtactaaaatatgagggtttttatataattgaatatgaggcaaatctaccattaaatgtactactactattaaatgtataaaggttacatgcagaattacattaacagtctctggcaataaaggaagacaataaataatatttagaactacataagtgtggacattacaaacaatagaaaatgcaccaaaactataaccattcttttatttgtataatgggattatgcatgatactatttcttttctctattttctgtatgtacttatcataggttggtaaatccataataaaaatatctgatacttgatatatctatgttaggataaaagtatcaagtcagcactgcttgaatataaggaaactcttcagagaaatctagttgtcctgcagctaatgatcatattacccaaagtactctgatatttacctttttagatttaagaaaactattatgatagtatatgaaactgatcaacactttgccttaaatcaaatatgcttattgctcatctatttcattatgaaagatacaaatataaataagtcatttttctagtcctgcagtagcttacagttgaaaagtgaggacagctgcgtacacagtaagtcgacacctgtattacaagtgccacctctttacttgaggaaggaggaaaggcttcaatagggaagtggagtgtgagctggagcttgagagatgtgaatgctagcaggcacagctgagggaggaacacggattcgttaaaacgttggtgcatgacatgcagggcgggttccagaaacaagtagatagggtgaggtaagcctttgtaatgggatgataaggtaagaaagataagttagaaaagatctgaagaacctgagatgccatccaaggaaatttggacttattatttaatacagaggaagctattgaagaattacatatagggaagtgacaagacctgcttgttcttttagtgagggaagttaggtggaggtgagaatgacggaatagaaaggagatttatttagagatcaaaacaccaattaggagattgctgcaatgtcccagaaagagaaggcctatatgtatcttcttttccacatttagctacacaagtcacataaaactgaatattttacaacttcttttcagccagtaaatactaccccattcaaaatattttcctctgtctaacttttatctttcatcctttaacttatgcttatctctttttggttctgtcttcagagaaggtaaagtactacaggtccttatatcttaaatacagaaaagcttcacaactcatgataattcagtaactatttttcaattatctgttaaaaagggacttacaaagcctaagagtttggattttaagggaactatatgaactatgtaagacataattttacaactcattgttttctgtattcaagaggcttcactttcaaattgcatgtgcaaaattattttgaataagttgttttttgtaacaactttcaatgtgcttcacttattttccttaaaaaatatatttttcaaatatattaacaccatactcttaaaagctgtattgcatatttatttttatttatctgcttttgaaattcaggtgtactttagaacaaaatagcttatataattttaataatttttctatatgttttcaaggaaattggacatgtgtatgtcccccgaccgtttttctttttctttttagctaagactttataatttttctcaactacattagtcaactgtatgaataactaaagacaacattgttcttgcaatttctaatttatcataaaatctcaactttttttattcactaattttgtctgacctaattaatgatattatgcccttcaaactgaaatttacaaaagtcaaagctgctttttagaggcctattcctttttaaatgtgttcatgctcatattcaccagtggtttgtatagtttacttgtgtatcaaatgttactttccatttcagatctgctcaatattattagaaatgatacagaaataagttttacagatctgtagaggaagatcacatttctctctcttttttttctttacttttaattttttaaaaacatttcctaccaagaatcttgaaaaagagcacatatatgggcttcttttttataagtgttcgcagactagtatcattaacttcaccctgggaacctgtagaaatgcaaattcttaggcctttccccaaacttactaagtcagactctgctattggtgtttttaacaagacccctgggtgattttgaaactcatgaaagttcgagaattactgattcattgcatagagcaaggctgaactgtgtagacatttttatatgtaaataagaaaattgtgttgctttttctgtataggtctcactggttagcttaactgcaaatgccttgggttactcagaacttggtgccatcaaatccctcagaacactaagagctctgaggccactgagagctttgtcccggtttgaaggaatgagggtaagactgaatgccttagagtttgtcagaattattattgagagcagactgacactttgtaccatggaaatgtcaaatttatggagaatttgtgtcttacacattcatactgacatagctaatcaatcaaaaataatatttaccagatgcccataatacttggcactgctggagtcactcacagagtagtatattgccagagggattgtttctgattagctagattttcacttcttggaaaatctctatagttatgctgctgatttgaatcaagattatttatgttcacttcatttataaatgtgcaggaaatcctactcgctgtagtttaagcctaccaaatcattgctcatcatttcttcactactccgctgtgatacactttgagccttttgatgtttgaatcaggccttttagttcttaaacacaggctgaaatggctaaaaagtaggtcaactggaaatctaacgctcatttagaagggtggtacaaaagaacagaggagtttgtgctgacatttgtcgtcccctgaggcacaaaacctgagaccacataccctcaccacctagaaaatgatgatgccttgtctcagttgttttagctggttcaaagaggattttaaaaaaatgatactttttgtgatatttgaaaataagttgcttagactttatctgcatgttatagtgatactagctcatattttctaactaagaaaatagttacttagactttatctagtgttacaatcacaactagagatgaatggtgtgtgtagatgtgtgtctgtatatgcatggttacatagaaaagtgttattagcggtaaaattctttttactttaccaattagaaagaacagtttttgcagtagaaggcttaataaacaaaaggtatcaatctttcagtaccagaatactgtttatattttctgtgtggaatttgatccccaagtggtctcttttactctcaaattttggacagcaaattgtatggtttgtatgattttttgaaagtgatgttcacttctatattcatgccactgtttatactcttaattatttttggcatttgctgttagttccatcctttgaggtaaatttgctacatgtgtgttattacctcttgagaaaacattctccaatataaaattcgttgtatactcttctgatttataattttaaaattcttagttggagctaccagagtctagtttctacccaatattcaactttgaaacagatttttttaatcatttgactgttcttttaataatgtttaaaaataagtaaatatttgttgttggcttttcacttatttttccttctcatcctgtgccaggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagtaagtaatcactttattattttccatgatgtgtaattaaaatgagtctaaagtttttcttcctcataatgagatatccacctgttagaatggctattatcaaacagataaatgacaataaatgctggcaagaatgtgaagaaaagggaacccttgtacattgttggcagggatgtaaattagtatagcttttatggaaaacagtatggaggtttctcaaaaaactaaaaatagaaccactatgtgatccaacaattccattactgggtatatatacaaaggaaattaaatcaacatgtcaaagagatgtctgcactctcacactcactgcagcactattcacaatagccaaaatatggaaacaacctaattgtccatcaacagatatgtggataaagaaaagtgtgtgtgtgtgtgtgtgtgtgtacatatatgtatatgtatatatatacacacacgtatttctatatacacacgtatagatatacactgtatatgtatatatctatacacatatatagacatacacagaaacagtgtttgtgtatgtgtgcgtgtatatagaagtagtcagggaaggggcagagcctgtggcactaagaaactgagaaaatgtacaagacttttgttttcagaattactatgtccgcacaacagaaaaagtatttcaaaaagtaaatgcgcttgaatgtatttgttttcagtttaggaaactgcttctttttgtagagtgccttaaaatagtatgttcaacaatattaaaaagattttcaaaaataagccctcgtgattgatgattggtaataatcatttaaaaacttattggatgtatatatatgtgtgtgtatacacgcacacacacacacacacacccctatagacatacacaatgaaatagtattcagcctttaaagaagaaggaaatcctgtccttttatacaacattgattcacctggaggaaattaagtgaaataagccaggcacagaaagacaaatgacacatgatgtcacttatatatggaatctaaaaaacacaaactcacagaaacagaaagcagaatgacaatcaccaggggctgggggatgatgggagatgttggtcaaaggatacaaaattcaattcgacaggaagaatacattctgtagagctattgtacagcatggtgactatagttaataataatatattatatacttgaaaatagctaagtgagtagatatgttttctcatcagaaaaaaataagtataagaagcgataattatatattacttagcttgagttagccatttcacaatatatatatatttgaaaacatcattttgtacaacataaatatattcatttttatttgtcaattaaaaaatgaatatatttttgaaaagcaattaaaataaaaatgcatatacattttaggaactctatatagatgcactaaaactatataaaaatgatataatactatacaacaataaaataaaatttttcttcctctgtgtttacaaatacttccttaggcccatctgcctagattcctcttaccatgattgaactatcttttctgccccacgctggaaacatgatggttctaaaaactttattgtctccctgactatgcatttggtagcatagccaagtcctttgttactgggagtttaatctaggcactcattgttttcctcccttcctactctgaggaaagaagtgctggccccaaggggggttgaaaaggggtgtgtgtgtgtgtgtgtatgtgtccacacgcgtgtgtgtagatagagaaagagagagagactttcaaataggaaaattgctctcttgcaaatgaaaactttccaattaagactattgtgtctgctatgcactcataataattcattcagctattcaactgactgcagtattaaatctccactagctcctggacacaatccacttacacgatcctcaagactattaaaatagtcaggaaaggggaagagcctgtggcactaaggaactgaaaaaatgtacaagagttttattttcaagatcattatgtcaacggagcagaaaacaaatatttaaaaaaggaaatgcagtagaatatattgttttcagattaggaaactgcttcctcttatagagtaatcacctcaaaatagtatgatcaacaatattaagaagattttcaagaataagctgtcatgattggtgattggtgtaataatcatttagaaaagaataagtagaaaggaagcattaagataaataatgcagcatacttttgagcttgtctcatgctgctactatacacatgaaattttttcatcaaagttcatgatatatttttatataaacacatcagagtcaaagattgttcatattgtttttatgatagcatattgttacagtagatcattatttaattatatatgctaaatatccacataagatgttatagaggaatataaatttgaagtattttcaatgcatatcgcaaaacattgccccaaaagtgaatacaaatttcaagcttatttatatgcctgtattgaatacatgtcaaatagaattttgatcaattattcaatttattttctaaaattataattttgggaaaaaagaaaatgatatgacttttcttacaggccacgtttaagggatggatggatattatgtatgcagctgttgattcacgaaatgtaagtctagttagagggaaattgtttagtttgattaaatgtatatttctacaatattgtaatttagtgatattgtcaataaaataaaattatgtgcttaatttataaaacccatctatattataaggataaaatatttaatcatactatttctttcaaaattatcataggatgattttctctaatcactctgtatcttttaacatatcttttctagtatttagcaaggcacctgacacaaaactttattgtatgtattttcaaaatgagacattttatttttggctctgatagtcctggtcatttgtgcattagaagttctcacaggcaatattttttatctgtaatatatttcctccagcttttgatcttccttataataggaaggatatgactaaaaacggggacaaaaataaacaatttagtgtttctcttgggaaagtgagattaagtggtagaagggagggacttccctaatctactttatacataccagtactttgaattcttttctataattttcattaatttctcactatttaatgaggaatgaagtcacattttgaaaaaaaaaaaaaaagagattgatttctggtatgccagagcatgataataaagctcaaaatgctctttccctagcaccagcagctagctttctgagtgaagaattcctgaggtttttttttttcttttttccacttcataaaaacagagagggagcaagaaagcatgaaaagccctgcattgtatctctataagtgctatcaggaattccagttatgagatttttctgaatagtaataataatttattgattatcactattcactgtgccaaggactttctcacattatcccatttaatcctaaatgacaaccttattgtataggtgatactagctctattttactactgaagcaaagaggcttaatgcgttaaatgggaaaacaagtttttgaaccctgaccacaaataatggctcatacccactttccacagtggttcttacctttttgattaattaattcaatgctctctccaccttccttatcaatagcttatatgccatgaaacattttcagtttcttctttaataacttagcagaccttttccgctgcaaaactcctggaatttccagcacattacaaaagatgaaagccaattgagcactacatttatgaaaagttgctggatcttgaactttaattagtaaattgcatcagataaatgcaaatttaaaccaaaataaaacattatctacacacctaccagattggcaataccaaaaagtctgacaataccaagttttaccaaggataaacagcaataagaacactcgtacaatgctgataggaaaaaaaatagttaaataatcctttaaaaacagttgggtatgatcacattatttgagaaagttaaagatattttttaatactgcaattctactttgaacaacgtatcctaaagaaacttatgcacatgtttaggataatctatgtacaaaaatgaatataacttttttttgcacttgcaaaaaactgggagcaactcaaaaacagtagaaataggcaaataattgaatactatatagtgatgaaaatgaatgaataccgccatatacaaccacatggatgagccttaaaaatacaacattgagttaaagaaactagacacatactataattctacttatataaagttcgaaattgacaaaactaagcttattgttcaaaactgcatactgaggtgttaacttgaaagaaaaagcagggacatcattaccataaaagtcaggataatgattacctccagcagggatgatggagtttatgtttgagaagggtacaccaagggtttctgaagttgtagcaatgtcctgggttatggatttcacttataaaacatattatattttgcatttatgtattatgcactttcctgtatgtatattgtcttttaaaaattttaaaaatataattttacatcactgttaactaaactcacatacacaaataaaatctcatcgaagaatagcagttttacaatattcctgatattttccattttgctgtatttccttagaaacaaaattatgctggtcataatcctctaaattgatttcataacacagtgggttataacttgcatctattatcatcatcagggattggttaactgagttggttagaacaatgtcctattagacctgtgaaagcttacagctaaggcgcaaacctactatcacacagttttctaaacaaaagtggattagacaagagatagtatcattgttacagaaacagtccctactgaataggataaagcaatagattcattttcagaaaggaaagatcaacctatatacctacatgcagacctactacaatgattcttgcctatctaaagaaatgtattataccaaacccttacacttagcaattactactggccgccactgttctaagcatatttatatgttaatatagttaatcttcacaaccacactatgaggtttaagtttgattattttcatctcacagatgagaaaactgagtcagagaaagtaaatcttaaaagttttgacatagaataatgtgacgctgacatctcttttgtaagaagaggaaatctttaatttgcatgctgtgttgggaactttgcttagaaaggaaagtgcattcataatctgggcatttgttgggtgaaattgtctataatcattcagacttctatatggttatttcattttcccaggtaatgaatagtcttgcagaactcttcaataagcatgtgagatttgaaggttcataaaatctgtttagtgtttggtttattttcattccagagattaaaacatgcttagataattaaaaactcactgatgtactttttgtgaaacaagtactagatataatggttacaattcttcatattctttaggtagaattacaacccaagtatgaagacaacctgtacatgtatctttattttgtcatctttattatttttggttcattctttaccttgaatcttttcattggtgtcatcatagataacttcaaccaacagaaaaagaagataagtatattaaaacttcatccttgctctgaaatatgaactaaatatttcatactctttcctttagcctccaaaatgcaatcaccaaaaaaagaatataaaattcagaaattattttgagacatttgataatcgataagcttttaagcaattaataattcagatagcatgtttttgatatttttagtctagaaatatgactaatatggcataatttatatattgaataaaggcatctctataaatacagatattagtaacaatagaatgaaatgtgggagccaattttcacatgattactaaggtggattttatagccagcaaagaacacaattttaacaagtgttgctttcatttctttactttggaggtcaagacatttttatgacagaagaacagaagaaatactacaatgcaatgaaaaaactgggttcaaagaaaccacaaaaacccatacctcgacctgctgtaagaataacatattttcattgcctgttaaaactatattacctaaccgtttcacagcccgaatttctagaaactagttatttttgtggatttgtaacacaaagttttttaccttaacaatgggactagctagcctaaatagcttgaaaaatgtactttacatatataatatgtataaattatataatgcataacatattttatatgtaaacatataaaatacatagaaataaaatttgctatacttaagtgccagtggtatcatacaagctgatgtcattaagacacttctaataacatcaaaaataaaatacatacatacataatgtgaaaatattaaatgttctcagagtacagaggagacagatcggaataattggtacgtcacagattggcctcagtttttgtccaactctgcagattgaatggaatcattaatgaaacaggccacaggttttgcttttttctggttaaacaaaaaaaagacaaacctcatattttcccctactatcccacccttaaatgagatgatatcattctttgtagggctttttattggctcttccaggtgtacatttgccagtgatactgttcgttcagtttggctgctgcagggagttgctgccaggagaatcgctaagtttttctatcactcctgaaggactagctcatatattaagtctcagaaaatcttccccaacgtatacgtggtataaaacacttcagtgtttctcagaaatcttgactctataaatctattggtgacaatataaaacagaccgtaattaagtgttcagttggtaagccggccaataactcaaagaaaatggatagctatattgggtcaaacacaaagggtgtacaacttgagcctagtctttaggaaataatacaatttgaatgaatagagagagaagcagagaacatttactgtatgagaaaatgtatacttcatagccatatagacaaatatatcagtgcagaatagtgatgcatttgaattagtgagtagtagacactggttttccgagttacatgagacaaggttaccatacgagtctgaagaaatttgttctaattaagcaatacaaatgcaatatagttaacagaacagcctagtaatgtgaaaagaaagattttagagagtttaacctagagactggtgtggaacaatattagaggcaaaataaccctcggccatagacaagaagataaacccttacatacaagaagatagtccataatctgtgtccaaccagcaggactggaactactccaggagtgaagttagccaataagaagactcaattgggatgaaacacaggaaaagagggaggatgcaatgaaaaaactgggttcaaagaaaccacaataacccatacctcgacctgctgtaagaatagcatattttcattgcctgttatgaaacacaggaaaagagggaggatatgtaaataacagagaatctaaaatataagctagttgatattttgtgaaactgttggttccactatcatatactgaagtcatatgaaggcactgggaaaaatagtgttagagcctatgaaatgtccagactgaaataaggattttagcattgtcagaacaaaattcaattgagctctgaaacacagattcatttttgaaaaataattagaatagagaaaaaaacaaaattctcagaatgaggccttgcatacttcatcaagatataggaagaaataaatcaatgaagaaatgagcttgagtttgtttccatcaaatgacatggatttacctgtagtggtaggggtgtgtggaaaaagttcaacacattcagctagaatattatcagtgtcaatttggcaatttagcaagtaactagtaaaatccatttattcctgcattgacaatatgtactatgtagtatgctaagcatttgaacttaaatatcgaacagtatggagtctagttaatgcaacggatagtaatcaaatagtcctgccaaaaaatggaagtatcccagaaaaaaagggatactttcagctgtgagagctgattagggggaaggggctgattaatcagggaagttagggaaggctttattaaaaaaatatactagctgaggatggaaaaagaatagaaagcatcaatagccagagtgggatgagaagagccctgtagaaggggaatgaatttgtgaaggtccttatgtaggagggctggtgagactggagtgcagaaagtcaaggttcatttgggacacactgagaataaagaggttaggataagcccaaacttttctgggccttggaggccgtgttaaggagtagttttcatcctaagagcagtaagaaaccgttaacgtggacccagtcagtctgggctttgtggtgatcactcaatcagtttcacagaggccgtgtgaatacattgtagacttgttttggagctatttcagagatggtaggtagcctgaaccatagcaatgtgcagattaataaaagtggatggatttgtgagctatcaccagagtgaaatttaaaagtttgtctattaattgaatatgggaactaaagaaggaaccaacaagaatgactggtgtctttctgctttgcacaactggataaatactgatgtcatgcaggaaatgaagaagggacagaaagtggtgagaaaattggagatgctagtttgcagaatttggcaaacgagtcagagtgagagagtgagaggaaggaggaagggagagaaatgatgaatatttagaagtagcaaaataaaggtttcttaagattcagagattaggtttaaaggaaagcaaaaggaattttagagaggaaaagatcgaagacagagggaataattacggcataaaaatgcacaagatgtgggacaaggacatagtggtctagggtagctttagaaagaaaaaggggctgagtcctctaatgaatttggagtaatatatgaaaagaacatggaaattaaaataatatgaaatgcaaaaggaaacagaggagtttatttaaactgtttaaatttaatgttctaaaaaaagtaaaaatagagggcaagagaatggaaatttatgagaagtttggaattatctttggagcaaatgaaggacaaaggattgctaattgttaaatctgaagggccaagatgaagttagagaacataaatttttggtgaataagatcttcagaattatacatcttgttccagcatatttgacaccctaggatttaaatgggagaacagaacacagagactcgagactggagttgtacattgagatgtctgtctcattggacaactctatgaacagggaatctaaacagttttttattagtcatggtgatattaaaattaagaccaaatttctgcttttaagatattttgaacttactatactctaggagccatatctgagagaaaaatgatactgctcctgcttttgaggggcctcaaaacaagtggaagggaaagaaaactaaaattgaataagagcaaaccatttgcaatacaatgccatacattttatgatcaatgaaagcactcagttttctgagagcactaagtgctttaaactcaaaactgagttagaattcatgagacagagaaggagtgggggacatgtgttttagacagaatagtagacagataaactatgtaaaatgatacagtagaacctgcctaagcttctaagagtggtaggcaggaaatatcagagggtggaagtaaggggaagatgccagacttggaaagttaaactacagtaaattaataattaataaggaagggtttgaactaaaagtagacacatttattgggttgaaaaaggccctgagaagacagatccagctggagtaatagaaatctagttcagcaggccaaatacgcattcagagaaaagagcaaaacaaaatagaacaagtagtgtttgatgtccaaaaatcaccattggaagtaatagaagcctcccaatagaaagagggcagaacactaagatgtagaatccaggccactaaaagtgtcaggatctgggaaagcaagccattaggtgtatatgtagcagagtattagtcattctagttgagaaggtagagaaaggcagcccaacagaggttaagtcaagaccagatccctagattacctgagaaacaaagcagatacgtgcaaaatggaacaatacagaaaccaatgatcagaactggtttacaagttggggacttcatttcataagcaagacataaggcaattagtacttggaaataaggtccaaatagactagggcaaagattgaatatttccattgtgactttttaaaagataattttattcttacagaagagttactcataatgaatactctaatgaatctatacacagtgtcctcttgttttaacatcttatgcaaccatagatcagttctcacaactaagaaattaatcttgatataataccattaaataaaatacagaactgagtcagatttcaccagtttttccactgaagccttttctctagaatgatgatttttaaaacatcttagctgaactttaaaatgaaatttaagatgctgtagctttagtgagagaatataaagtcagaaatcagacgaaaaatttaaaaagagagaggaaaacttggagaagtatttatttattagttgcttaaagtaaaattaataccctcccaacacatgggataaaaaattttattacatgacaaatatttactaactgtccgtcataacatgatggtgttctgtgcactgagaacataatacgtgagtttataaaacctggtatcaatgtgagtataaataaaacaaatacatttgaatacagttgaatatacaatatacaaaattttcttccaagtataaaacgaaaataaaatacactactttctttaatagaatagaacattgtaataatgttccattgcatttgaccctcacataaatgctatgaggtagcattaagagataagatttgaggctgggcatggtggctcatgcctgtaattccagcactttgggaggtcgaggtgggcagatcattaggtcaggagtttgagaccagcctgaccaatacggtgaaatcccgtctctactaaaattacaaaaagtagtcgggcatggtggcatgtacctgtaatcccagctactcaggaggctgaggcaggagtatcgcttgaacccgggaggcagaggttgcagtgagccaagatcgtgccactgcactccaacctgtgcaacagagcgagactccatctcaaaaaataaaaaaaattaaaaaaaagagagagagataagatttgagatctgacatggagcttccctatttacactacttacctgctttgtgacctaaggcaagttacctcagctctccaatcactggttttgcaaggaatttttttttttgtaaaatgttgtgaggattaaagatgtgtttttataaaagctacattttttgttgctttcttaaaatcagaagaattgaattcgattttttttaaggtttctaatggaacttttacatattatttgttccagaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggtaagaagaggtgcttttattcagttaaggaatatagtggtaaaaatatgtgttttaaaactttagaggtgtttttcactaatctttctcattcatcccaaactcccaaataaaaatctaatagtccattgttttagttttagtttgccatttctctaattgcatgctgtgcttgaaatgatgagtggaatacaaggaatttatattttcagctttcatttattctcatttaatattttcatctgttctcatctcagaagacaataactgcaactttggtagaatagtcttgtacctggtcatactcctgtggtattgacagttactgctttgaataaacaatcaatccacacacatatatacataaatcatttgaagtagtcacataattcataaatatgacctcttaaataattggaatagtgtatatgtgcagttatatatataataacacatatataagtttcatgttatctttgggtgcagacagttttctgtggtttgcaatatctctttttggaagcagatagtttgtttgaaaatccaaaacagatttgttatcatcaatgatacattaatgttaggatacatacatacattaagtcctaggaatgcaaaagatttattggaaaaaatatatatatacagtgtttatgtataagatattaaatgaggtactggaagtaaatataagaagatttaagagaaggttctacctatttggggaaacagaacattcacatggaggggaaaattatatagcactctttaaactactttctttagtcgaatagaacattgtaacaatgttccactgcatttgattctcacataagtgctatgaggtagcattaagaggtaagatttgagatctgacctggagcttccctatttacactacttaccttctcagtgacctaagagaagttacctcagctctccaatctctggttttgcaaggaatttttctgtaaaatgttattgtgaggattaaatcagattatgtatatatatgcacttagcactgtgcctagcatgaagaaaagacttagtaaatgttcagtttgaccacaagaaaaagttgatattatcaccatttactcatgcataaaagcaagtgccaggattcagtcccaagtacatctgtctccaaagcctatgttttcttctgtacatcacgctgcctactcccaaataacatagaatctcagaaagtaaagaactctcatattcctgacccaaaatcatacacctttagttcttatgcaaatactagaactagtattttggacatataaattaatttctgtacttggccactgtatgcttcatgatgtctttggaccttccagggttgagtcatttttttgatagatgctttccttgaactaggaaaaatggcccttattatcttcatttaatataaagatgtaaatgttataacaccaaacataccagtttcattttgctcaacaaacattgcagattatttgcatatatacatgtacctaactgtcctgttcacattttgtaaaactaatgtacttatgtaaactttcatttgctactattaagtataacaatatttttgttatttgttgattttctacaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaaaaagaaaccaagaattttccattttgtgatcaattgtttacagcccgtgatggtgatgtgtttgtgtcaacaggactcccacaggaggtctatgccaaactgactgtttttacaaatgtatacttaaggtcagtgcctataacaagacagagacctctggtcagcaaactggaactcagtaaactggagaaatagtatcgatgggaggtttctattttcacaaccagctgacactgctgaagagcagaggcgtaatggctactcagacgataggaaccaatttaaaggggggagggaagttaaatttttatgtaaattcaacatgtgacacttgataatagtaattgtcaccagtgtttatgttttaactgccacacctgccatatttttacaaaacgtgtgctgtgaatttatcacttttctttttaattcacaggttgtttactattatatgtgactatttttgtaaatgggtttgtgtttggggagagggattaaagggagggaattctacatttctctattgtattgtataactggatatattttaaatggaggcatgctgcaattctcattcacacataaaaaaatcacatcacaaaagggaagagtttacttcttgtttcaggatgtttttagatttttgaggtgcttaaatagctattcgtatttttaaggtgtctcatccagaaaaaatttaatgtgcctgtaaatgttccatagaatcacaagcattaaagagttgttttatttttacataacccattaaatgtacatgtatatatgtatatatgtatatgtgcgtgtatatacatatatatgtatacacacatgcacacacagagatatacacataccattacattgtcattcacagtcccagcagcatgactatcacatttttgataagtgtcctttggcataaaataaaaatatcctatcagtcctttctaagaagcctgaattgaccaaaaaacatccccaccaccactttataaagttgattctgctttatcctgcagtattgtttagccatcttctgctcttggtaaggttgacatagtatatgtcaatttaaaaaataaaagtctgctttgtaaatagtaattttacccagtggtgcatgtttgagcaaacaaaaatgatgatttaagcacactacttattgcatcaaatatgtaccacagtaagtatagtttgcaagctttcaacaggtaatatgatgtaattggttccattatagtttgaagctgtcactgctgcatgtttatcttgcctatgctgctgtatcttattccttccactgttcagaagtctaatatgggaagccatatatcagtggtaaagtgaagcaaattgttctaccaagacctcattcttcatgtcattaagcaataggttgcagcaaacaaggaagagcttcttgctttttattcttccaaccttaattgaacactcaatgatgaaaagcccgactgtacaaacatgttgcaagctgcttaaatctgtttaaaatatatggttagagttttctaagaaaatataaatactgtaaaaagttcattttattttatttttcagccttttgtacgtaaaatgagaaattaaaagtatcttcaggtggatgtcacagtcactattgttagtttctgttcctagcacttttaaattgaagcacttcacaaaataagaagcaaggactaggatgcagtgtaggtttctgcttttttattagtactgtaaacttgcacacatttcaatgtgaaacaaatctcaaactgagttcaatgtttatttgctttcaatagtaatgccttatcattgaaagaggcttaaagaaaaaaaaaatcagctgatactcttggcattgcttgaatccaatgtttccacctagtctttttattcagtaatcatcagtcttttccaatgtttgtttacacagatagatcttattgacccatatggcactagaactgtatcagatataatatgggatcccagctttttttcctctcccacaaaaccaggtagtgaagttatattaccagttacagcaaaatactttgtgtttcacaagcaacaataaatgtagattctttatactgaagctattgacttgtagtgtgttggtgaaatgcatgcaggaaaatgctgttaccataaagaacggtaaaccacattacaatcaagccaaaagaataaaggtttcgcttttgtttttgtatttaattgttgtctttgtttctatctttgaaatgccatttaaaggtagatttctatcatgtaaaaataatctatctgaaaaacaaatgtaaagaacacacattaattactataattcatctttcaattttttcatggaatggaagttaattaagaagagtgtattggataactactttaatattggccaaaaagctagatatggcatcaggtagactagtggaaagttacaaaaattaataaaaaattgactaacattttaagttgtgcatcttttctccttcctgtccacctattgttctttttttcacttttccatttcaatttcttccttatgtattcttgatctacttttctttatatccttctatcctttccttgcgctctcagtatttttcatttaggatattctccttgtttcttttctgttcaccaaatgtcttgtttattacagcctatagatcacttagatttagatccctaaaatttgctgtcactctgtaaagtgcacatagtatctgcgtatttatttttacaattttctttacaaaaattctaagaaagcttagactgcaacttttcataatatataaacaaaattccaaaatagcacagatttgactttctcatttaattatgaacagtttgggaaatgtttctcaattctgaatgcatattagaactacctgagaagcaaaaaaaaaaaaaaaaaaaaagccagtgcatgggccaagaccaggaccagaccaattgaactcatatctctgggtgagacttaagcatttaattttttgaaggctcgtcattgatactgattcataccaaggactgagaactagcaaatttgggtctcctttgcccaataggaaaacatattctccggaaagtccaaactgtccttcattacataacttatcatgtacagaataaagttcctaagaggatggctttcggagtatttatattgcagattgagggattgacattgatctaattataatagaggttcttctttagggctgtagttggtataaagggaattctattagaaaagtttagggagactgtgttcaggtaacacatgcacacacacacaccccatatggcaccttaactatccctagtgtatactgggtggaatgaatgtgggatggtctaggaaacagtataaatttataaaatagttccctccagtgaatgctacatgccctgttgcatgagaacctttaatctggaccaatagaaggcagttcatggtggccagtcagaaagccattcaatttgtcattaccaactcacaaatctagacaggaaagattctcattctcatcttctccattcttggcgcatctcacacaatcccttgtttttctgagctctgtaacatacagacctatttacaaacagaagggaggagacaagagaggtaaggggaagtaaagaaagaggagaaaggaggagagggaacgataggggaaagtaggagaaaggaggggagtgtaagaaatgggatgtttcatgaattataattctgaaaaaatgtctggtgtggatacctccagcaatggtgagggaaggaggaagaaatgttgcttagttgtcaatatgttagatctaaaatataaacatgtttcttctcttatttatttttatttgtatttatttatttatttatttttgagataaggtcttgctctgtcacccaggctgaagagcagtggtgtgatcatggctcactgcagccttaaccttctggactcaagtgatcttcccacctcagcctcccgagtatctgggactacaggcatgtgctcccacacccagctcatttttttttttttttttgtattttttgtagagacagggtctccccatgttgcctgggttggtctcagatttctggactgaagcaatcatcctgcctcagctttccaaagtgctagaattacaggcatgagccattgcacctgcccctctcatttttttaaagaaaagatacctgtgaaaaaagtgaagaggacattatacaagaaaatgttacaatgattccatggattcaggaatatttttttttcttttacctgaactaaatacatatttgagtgtctactatatatcaactttgcaatagcagctaagagatcaaagaggaatataactatccaaagaaaggctataaccagaggtgaagaacctggaaagccatgctagagtttggaattgattctgtggcaaaaaggagtgattactcctgagaagtagaaggaacagtgaaatatgacacatctgtagctgctgaatctggtagtaataaggctatgctgcccattttttctagtattttacagcaatctcattttcccaattatttaaatgggttcttatctcttggtatagtatatagatttattaagtaaaagaatataactagatttttaagtcatccaatgtgacacttcctgctaatagcctattttaatcaagttacattttttatatttacagatacatctaattttattcctatcactttacttcacactttccactttttgaggtttttccttttattctcctcttccttcttgtcctcttttgctttgtgttttctgtttctctttcccccatctctgatggaaagttacacatcatctccctccattcatccctccctccctcctttccttcctttcttcttttttctttctcttttataagtgattactcttaaattttaacatgcatacttaattaaggtgaactgtaatcaatatttgtgtcctgatctggaaaaatgaattttcctagactgatgacatatcattgctctcctctaatatgttatttttgtgtagtatcttaactctaccttaatttgagataaaacaaattaaacagtattattacatgaatttgtaggcagtacttatttagacttacatactaattattg

SEQ ID NO: 3-2486 - skipped (2484 entries)

SEQ ID NO: 2487 - DNA, 20 bp, synthetic construct
  source 1..20: mol_type = other DNA
  modified_base 1: mod_base = OTHER; note = 2'-O-methoxyethyl thymine with phosphorothioate linkage
  modified_base 2: mod_base = OTHER; note = 5-methyl-2'-O-methoxyethyl nucleotide
  modified_base 3: mod_base = OTHER; note = 2'-O-methoxyethyl thymine
  modified_base 4: mod_base = OTHER; note = 2'-O-methoxyethyl nucleotide
  modified_base 5: mod_base = OTHER; note = 5-methyl-2'-O-methoxyethyl nucleotide
  modified_base 6: mod_base = OTHER; note = 2'-O-methoxyethyl nucleotide
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = 5-methyl nucleotide with phosphorothioate linkage
  modified_base 13: mod_base = OTHER; note = 5-methyl nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = 2'-O-methoxyethyl nucleotide
  modified_base 18: mod_base = OTHER; note = 2'-O-methoxyethyl thymine with phosphorothioate linkage
  modified_base 19: mod_base = OTHER; note = 2'-O-methoxyethyl nucleotide with phosphorothioate linkage
  modified_base 20: mod_base = OTHER; note = 5-methyl-2'-O-methoxyethyl nucleotide
  misc_feature 1..6: note = RNA
  misc_feature 7..16: note = DNA
  misc_feature 17..20: note = RNA
tctgcatgtaacctttatac

SEQ ID NO: 2488-2492 - skipped (5 entries)

SEQ ID NO: 2493 - DNA, 20 bp, synthetic construct
  source 1..20: mol_type = other DNA
  modified_base 1: mod_base = OTHER; note = 2'-O-methoxyethyl nucleotide with phosphorothioate linkage
  modified_base 2: mod_base = OTHER; note = 5-methyl-2'-O-methoxyethyl nucleotide
  modified_base 3: mod_base = OTHER; note = 2'-O-methoxyethyl nucleotide
  modified_base 4: mod_base = OTHER; note = 2'-O-methoxyethyl thymine
  modified_base 5: mod_base = OTHER; note = 2'-O-methoxyethyl nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide with phosphorothioate linkage
  modified_base 9: mod_base = OTHER; note = 5-methyl nucleotide with phosphorothioate linkage
  modified_base 10: mod_base = OTHER; note = 5-methyl nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 2'-O-methoxyethyl nucleotide
  modified_base 17: mod_base = OTHER; note = 5-methyl-2'-O-methoxyethyl nucleotide
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..5: note = RNA
  misc_feature 6..15: note = DNA
  misc_feature 16..20: note = RNA
gcataatcccattatacaaa

SEQ ID NO: 2494-2509 - skipped (16 entries)

SEQ ID NO: 2510 - DNA, 20 bp, synthetic construct
  source 1..20: mol_type = other DNA
  modified_base 1: mod_base = OTHER; note = 5-methyl-2'-O-methoxyethyl nucleotide with phosphorothioate linkage
  modified_base 2: mod_base = OTHER; note = 5-methyl-2'-O-methoxyethyl nucleotide
  modified_base 3: mod_base = OTHER; note = 2'-O-methoxyethyl nucleotide
  modified_base 4: mod_base = OTHER; note = 5-methyl-2'-O-methoxyethyl nucleotide
  modified_base 5: mod_base = OTHER; note = 2'-O-methoxyethyl nucleotide
  modified_base 6: mod_base = OTHER; note = 2'-O-methoxyethyl nucleotide
  modified_base 7: mod_base = OTHER; note = 5-methyl nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = 5-methyl nucleotide with phosphorothioate linkage
  modified_base 17: mod_base = OTHER; note = 2'-O-methoxyethyl thymine
  modified_base 18: mod_base = OTHER; note = 2'-O-methoxyethyl nucleotide with phosphorothioate linkage
  modified_base 19: mod_base = OTHER; note = 5-methyl-2'-O-methoxyethyl nucleotide with phosphorothioate linkage
  modified_base 20: mod_base = OTHER; note = 2'-O-methoxyethyl nucleotide
  misc_feature 1..6: note = RNA
  misc_feature 7..16: note = DNA
  misc_feature 17..20: note = RNA
ccacgacatatttttctaca

SEQ ID NO: 2511-2513 - skipped (3 entries)

SEQ ID NO: 2514 - DNA, 20 bp, synthetic construct
  source 1..20: mol_type = other DNA
  modified_base 1: mod_base = OTHER; note = 5-methyl-2'-O-methoxyethyl nucleotide with phosphorothioate linkage
  modified_base 2: mod_base = OTHER; note = 2'-O-methoxyethyl nucleotide
  modified_base 3: mod_base = OTHER; note = 5-methyl-2'-O-methoxyethyl nucleotide
  modified_base 4: mod_base = OTHER; note = 2'-O-methoxyethyl nucleotide
  modified_base 5: mod_base = OTHER; note = 2'-O-methoxyethyl nucleotide
  modified_base 6: mod_base = OTHER; note = 5-methyl-2'-O-methoxyethyl nucleotide
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = 5-methyl nucleotide with phosphorothioate linkage
  modified_base 16: mod_base = OTHER; note = Nucleotide with phosphorothioate linkage
  modified_base 17: mod_base = OTHER; note = 2'-O-methoxyethyl nucleotide
  modified_base 18: mod_base = OTHER; note = 5-methyl-2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 5-methyl-2'-O-methoxyethyl nucleotide
  misc_feature 1..6: note = RNA
  misc_feature 7..16: note = DNA
  misc_feature 17..20: note = RNA
cacgacatatttttctacac

SEQ ID NO: 2515-2533 - skipped (19 entries)

SEQ ID NO: 2534 - DNA, 18 bp, synthetic construct
  source 1..18: mol_type = other DNA
  modified_base 1: mod_base = OTHER; note = 5-methyl-2'-O-methoxyethyl nucleotide with phosphorothioate linkage
  modified_base 2: mod_base = OTHER; note = 2'-O-methoxyethyl thymine
  modified_base 3: mod_base = OTHER; note = 2'-O-methoxyethyl nucleotide
  modified_base 4: mod_base = OTHER; note = 5-methyl-2'-O-methoxyethyl nucleotide
  modified_base 5: mod_base = OTHER; note = 2'-O-methoxyethyl nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = 5-methyl nucleotide with phosphorothioate linkage
  modified_base 12: mod_base = OTHER; note = 5-methyl nucleotide with phosphorothioate linkage
  modified_base 13: mod_base = OTHER; note = Nucleotide with phosphorothioate linkage
  modified_base 14: mod_base = OTHER; note = 2'-O-methoxyethyl thymine
  modified_base 15: mod_base = OTHER; note = 2'-O-methoxyethyl thymine
  modified_base 16: mod_base = OTHER; note = 2'-O-methoxyethyl nucleotide with phosphorothioate linkage
  modified_base 17: mod_base = OTHER; note = 2'-O-methoxyethyl thymine with phosphorothioate linkage
  modified_base 18: mod_base = OTHER; note = 2'-O-methoxyethyl nucleotide
  misc_feature 1..5: note = RNA
  misc_feature 6..13: note = DNA
  misc_feature 14..18: note = RNA
ctgcatgtaacctttata

SEQ ID NO: 2535-2539 - skipped (5 entries)

SEQ ID NO: 2540 - DNA, 16 bp, synthetic construct
  source 1..16: mol_type = other DNA
  modified_base 1: mod_base = OTHER; note = Locked nucleotide with phosphorothioate linkage
  modified_base 2: mod_base = OTHER; note = Locked nucleotide with phosphorothioate linkage
  modified_base 3: mod_base = OTHER; note = Locked nucleotide with phosphorothioate linkage
  modified_base 4: mod_base = OTHER; note = Locked nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Locked nucleotide with phosphorothioate linkage
  modified_base 14: mod_base = OTHER; note = Locked nucleotide with phosphorothioate linkage
  modified_base 15: mod_base = OTHER; note = Locked nucleotide with phosphorothioate linkage
  modified_base 16: mod_base = OTHER; note = Locked nucleotide
tgggtctcttagcttt

SEQ ID NO: 2541 - DNA, 20 bp, synthetic construct
  source 1..20: mol_type = other DNA
  modified_base 1: mod_base = OTHER; note = 2'-O-methoxyethyl nucleotide with phosphorothioate linkage
  modified_base 2: mod_base = OTHER; note = 5-methyl-2'-O-methoxyethyl nucleotide
  modified_base 3: mod_base = OTHER; note = 2'-O-methoxyethyl nucleotide
  modified_base 4: mod_base = OTHER; note = 2'-O-methoxyethyl thymine
  modified_base 5: mod_base = OTHER; note = 2'-O-methoxyethyl nucleotide
  modified_base 6: mod_base = OTHER; note = 2'-O-methoxyethyl nucleotide
  modified_base 7: mod_base = OTHER; note = Nucleotide with phosphorothioate linkage
  modified_base 8: mod_base = OTHER; note = 5-methyl nucleotide with phosphorothioate linkage
  modified_base 9: mod_base = OTHER; note = 5-methyl nucleotide with phosphorothioate linkage
  modified_base 10: mod_base = OTHER; note = 5-methyl nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = 5-methyl-2'-O-methoxyethyl nucleotide
  modified_base 18: mod_base = OTHER; note = 2'-O-methoxyethyl nucleotide with phosphorothioate linkage
  modified_base 19: mod_base = OTHER; note = 2'-O-methoxyethyl nucleotide with phosphorothioate linkage
  modified_base 20: mod_base = OTHER; note = 2'-O-methoxyethyl nucleotide
  misc_feature 1..6: note = RNA
  misc_feature 7..16: note = DNA
  misc_feature 17..20: note = RNA
gcataatcccattatacaaa

SEQ ID NO: 2542 - DNA, 20 bp, synthetic construct
  source 1..20: mol_type = other DNA
  modified_base 3: mod_base = OTHER; note = 5-methyl-2'-O-methoxyethyl nucleotide with phosphorothioate linkage
  modified_base 4: mod_base = OTHER; note = 2'-O-methoxyethyl thymine
  modified_base 5: mod_base = OTHER; note = 2'-O-methoxyethyl nucleotide
  modified_base 6: mod_base = OTHER; note = 5-methyl-2'-O-methoxyethyl nucleotide
  modified_base 7: mod_base = OTHER; note = 2'-O-methoxyethyl nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = 5-methyl nucleotide with phosphorothioate linkage
  modified_base 14: mod_base = OTHER; note = 5-methyl nucleotide with phosphorothioate linkage
  modified_base 15: mod_base = OTHER; note = Nucleotide with phosphorothioate linkage
  modified_base 16: mod_base = OTHER; note = 2'-O-methoxyethyl thymine
  modified_base 17: mod_base = OTHER; note = 2'-O-methoxyethyl thymine
  modified_base 18: mod_base = OTHER; note = 2'-O-methoxyethyl nucleotide with phosphorothioate linkage
  modified_base 19: mod_base = OTHER; note = 2'-O-methoxyethyl thymine with phosphorothioate linkage
  modified_base 20: mod_base = OTHER; note = 2'-O-methoxyethyl nucleotide
  misc_feature 1..2: note = DNA
  misc_feature 3..7: note = RNA
  misc_feature 8..15: note = DNA
  misc_feature 16..20: note = RNA
gtctgcatgtaacctttata